SEQUENCE LISTING (832 sequences)

SEQ ID NO: 1 - RNA, 88 nt, Drosophila melanogaster
gagugcauuccguauggaagacuagugauuuuguuguuuggucuuugguaauaacaauaaaucccuugucuucuuacggcgugcauuu

SEQ ID NO: 2 - RNA, 80 nt, Homo sapiens
ugggaugagguaguagguuguauaguuuuagggucacacccaccacugggagauaacuauacaaucuacugucuuuccua

SEQ ID NO: 3 - RNA, 83 nt, Homo sapiens
cggggugagguaguagguugugugguuucagggcagugauguugccccucggaagauaacuauacaaccuacugccuucccug

SEQ ID NO: 4 - RNA, 84 nt, Homo sapiens
gcauccggguugagguaguagguuguaugguuuagaguuacacccugggaguuaacuguacaaccuucuagcuuuccuuggagc

SEQ ID NO: 5 - RNA, 87 nt, Homo sapiens
ccuaggaagagguaguagguugcauaguuuuagggcagggauuuugcccacaaggagguaacuauacgaccugcugccuuucuuagg

SEQ ID NO: 6 - RNA, 79 nt, Homo sapiens
cccgggcugagguaggagguuguauaguugaggaggacacccaaggagaucacuauacggccuccuagcuuuccccagg

SEQ ID NO: 7 - RNA, 87 nt, Homo sapiens
ucagagugagguaguagauuguauaguugugggguagugauuuuacccuguucaggagauaacuauacaaucuauugccuucccuga

SEQ ID NO: 8 - RNA, 83 nt, Homo sapiens
ugugggaugagguaguagauuguauaguuuuagggucauaccccaucuuggagauaacuauacagucuacugucuuucccacg

SEQ ID NO: 9 - RNA, 84 nt, Homo sapiens
aggcugagguaguaguuuguacaguuugagggucuaugauaccacccgguacaggagauaacuguacaggccacugccuugcca

SEQ ID NO: 10 - RNA, 84 nt, Homo sapiens
cuggcugagguaguaguuugugcuguuggucggguugugacauugcccgcuguggagauaacugcgcaagcuacugccuugcua

SEQ ID NO: 11 - RNA, 22 nt, Homo sapiens
uggaauguaaagaaguauguau

SEQ ID NO: 12 - RNA, 80 nt, Homo sapiens
ccuguugccacaaacccguagauccgaacuugugguauuaguccgcacaagcuuguaucuauagguaugugucuguuagg

SEQ ID NO: 13 - RNA, 75 nt, Homo sapiens
ugcccuggcucaguuaucacagugcugaugcugucuauucuaaagguacaguacugugauaacugaaggauggca

SEQ ID NO: 14 - RNA, 23 nt, Homo sapiens
agcagcauuguacagggcuauga

SEQ ID NO: 15 - RNA, 81 nt, Homo sapiens
ccuuggccauguaaaagugcuuacagugcagguagcuuuuugagaucuacugcaauguaagcacuucuuacauuaccaugg

SEQ ID NO: 16 - RNA, 82 nt, Homo sapiens
ccugccggggcuaaagugcugacagugcagauagugguccucuccgugcuaccgcacuguggguacuugcugcuccagcagg

SEQ ID NO: 17 - RNA, 23 nt, Homo sapiens
agcagcauuguacagggcuauca

SEQ ID NO: 18 - RNA, 110 nt, Homo sapiens
gaucugucugucuucuguauauacccuguagauccgaauuuguguaaggaauuuuguggucacaaauucguaucuaggggaauauguaguugacauaaacacuccgcucu

SEQ ID NO: 19 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 20 - RNA, 69 nt, Homo sapiens
gcugcuggacccacccggccgggaauagugcuccugguuguuuccggcucgcgugggugugucggcggc

SEQ ID NO: 21 - RNA, 23 nt, Homo sapiens
gagggucuugggagggaugugac

SEQ ID NO: 22 - RNA, 27 nt, Homo sapiens
cacuguaggugauggugagagugggca

SEQ ID NO: 23 - RNA, 20 nt, Homo sapiens
ucacuguucagacaggcgga

SEQ ID NO: 24 - RNA, 85 nt, Homo sapiens
ccuuagcagagcuguggagugugacaaugguguuugugucuaaacuaucaaacgccauuaucacacuaaauagcuacugcuaggc

SEQ ID NO: 25 - RNA, 21 nt, Homo sapiens
ccccaccuccucucuccucag

SEQ ID NO: 26 - RNA, 22 nt, Homo sapiens
ugagccccugugccgcccccag

SEQ ID NO: 27 - RNA, 75 nt, Homo sapiens
gugagggcaugcaggccuggauggggcagcugggaugguccaaaaggguggccucaccagcccuguguucccuag

SEQ ID NO: 28 - RNA, 88 nt, Homo sapiens
guggggccaggcgguggugggcacugcuggggugggcacagcagccaugcagagcgggcauuugaccccgugccacccuuuuccccag

SEQ ID NO: 29 - RNA, 82 nt, Homo sapiens
gugagugggaggccagggcacggcagggggagcugcagggcuaugggaggggccccagcgucugagcccuguccucccgcag

SEQ ID NO: 30 - RNA, 102 nt, Homo sapiens
gugagugacaggggaaauggggauggacuggaagugggcagcauggagcugaccuucaucauggcuuggccaacauaaugccucuuccccuugucucuccag

SEQ ID NO: 31 - RNA, 83 nt, Homo sapiens
gugagugggagccccagugugugguuggggccauggcgggugggcagcccagccucugagccuuccucgucugucugccccag

SEQ ID NO: 32 - RNA, 85 nt, Homo sapiens
aggccucucucuccguguucacagcggaccuugauuuaaauguccauacaauuaaggcacgcggugaaugccaagaauggggcug

SEQ ID NO: 33 - RNA, 22 nt, Homo sapiens
aacuggaucaauuauaggagug

SEQ ID NO: 34 - RNA, 22 nt, Homo sapiens
acgcccuucccccccuucuuca

SEQ ID NO: 35 - RNA, 70 nt, Homo sapiens
guggacucuagcugccaaaggcgcuucuccuucugaacagagcgcuuugcucagccaguguagacauggc

SEQ ID NO: 36 - RNA, 21 nt, Homo sapiens
agagaagaagaucagccugca

SEQ ID NO: 37 - RNA, 24 nt, Homo sapiens
agccuggaagcuggagccugcagu

SEQ ID NO: 38 - RNA, 63 nt, Homo sapiens
uacggaugagcaaagaaagugguuucuuaaaauggaaucuacucuuugugaagaugcugugaa

SEQ ID NO: 39 - RNA, 21 nt, Homo sapiens
agugaaugauggguucugacc

SEQ ID NO: 40 - RNA, 90 nt, Homo sapiens
gccuuugcagcugaugauacagcuucuuuccccaucagaucgacccuguugaucucuacacuauuggccaguuuugucugaugcauuggc

SEQ ID NO: 41 - RNA, 22 nt, Homo sapiens
acaggugagguucuugggagcc

SEQ ID NO: 42 - RNA, 24 nt, Homo sapiens
ucccugagacccuuuaaccuguga

SEQ ID NO: 43 - RNA, 88 nt, Homo sapiens
ugcgcuccucucagucccugagacccuaacuugugauguuuaccguuuaaauccacggguuaggcucuugggagcugcgagucgugcu

SEQ ID NO: 44 - RNA, 85 nt, Homo sapiens
cgcuggcgacgggacauuauuacuuuugguacgcgcugugacacuucaaacucguaccgugaguaauaaugcgccguccacggca

SEQ ID NO: 45 - RNA, 18 nt, Homo sapiens
aucccaccucugccacca

SEQ ID NO: 46 - RNA, 23 nt, Homo sapiens
caagucuuauuugagcaccuguu

SEQ ID NO: 47 - RNA, 22 nt, Homo sapiens
caggauguggucaaguguuguu

SEQ ID NO: 48 - RNA, 21 nt, Homo sapiens
ccuguugaaguguaaucccca

SEQ ID NO: 49 - RNA, 22 nt, Homo sapiens
cuggacugagccgugcuacugg

SEQ ID NO: 50 - RNA, 22 nt, Homo sapiens
ucggauccgucugagcuuggcu

SEQ ID NO: 51 - RNA, 22 nt, Homo sapiens
cugaagcucagagggcucugau

SEQ ID NO: 52 - RNA, 23 nt, Homo sapiens
cuggagauauggaagagcugugu

SEQ ID NO: 53 - RNA, 86 nt, Homo sapiens
cacccagaucagugcuuggcaccuagcaagcacucaguaaauauuuguugagugccugcuaugugccaggcauugugcugagggcu

SEQ ID NO: 54 - RNA, 18 nt, Homo sapiens
gucccuguucaggcgcca

SEQ ID NO: 55 - RNA, 17 nt, Homo sapiens
ucccuguucgggcgcca

SEQ ID NO: 56 - RNA, 17 nt, Homo sapiens
gugggggagaggcuguc

SEQ ID NO: 57 - RNA, 20 nt, Homo sapiens
uaaagagcccuguggagaca

SEQ ID NO: 58 - RNA, 82 nt, Homo sapiens
ugagcuguuggauucggggccguagcacugucugagagguuuacauuucucacagugaaccggucucuuuuucagcugcuuc

SEQ ID NO: 59 - RNA, 20 nt, Homo sapiens
ucguuugccuuuuucugcuu

SEQ ID NO: 60 - RNA, 22 nt, Homo sapiens
ucuauacagacccuggcuuuuc

SEQ ID NO: 61 - RNA, 21 nt, Homo sapiens
ugcaggaccaagaugagcccu

SEQ ID NO: 62 - RNA, 75 nt, Homo sapiens
gaggguguugaucagcagaucaggacuguaacucaccauagugguggacugcccugaucuggagaccacugccuu

SEQ ID NO: 63 - RNA, 23 nt, Homo sapiens
uggaguccaggaaucugcauuuu

SEQ ID NO: 64 - RNA, 21 nt, Homo sapiens
cuuuuugcggucugggcuugc

SEQ ID NO: 65 - RNA, 19 nt, Homo sapiens
uggauuuuuggaucaggga

SEQ ID NO: 66 - RNA, 24 nt, Homo sapiens
uggcccugacugaagaccagcagu

SEQ ID NO: 67 - RNA, 66 nt, Homo sapiens
ccugggaacggguuccggcagacgcugagguugcguugacgcucgcgccccggcucccguuccagg

SEQ ID NO: 68 - RNA, 22 nt, Homo sapiens
ugugagguuggcauuguugucu

SEQ ID NO: 69 - RNA, 92 nt, Homo sapiens
accuaccuaacuggguuagggcccuggcuccaucuccuuuaggaaaaccuucuguggggaguggggcuucgacccuaacccaggugggcugu

SEQ ID NO: 70 - RNA, 112 nt, Homo sapiens
agacgaggaguuaagaguucauucggcuguccagauguauccaaguacccuguguuauuuggcaauaaauacaucugggcaacugacugaacuuuucacuuuucaugacuca

SEQ ID NO: 71 - RNA, 18 nt, Homo sapiens
uugagaaggaggcugcug

SEQ ID NO: 72 - RNA, 22 nt, Homo sapiens
uuuagagacggggucuugcucu

SEQ ID NO: 73 - RNA, 22 nt, Homo sapiens
uuuucaacucuaaugggagaga

SEQ ID NO: 74 - RNA, 89 nt, Homo sapiens
ugcugcuggccagagcucuuuucacauugugcuacugucugcaccugucacuagcagugcaauguuaaaagggcauuggccguguagug

SEQ ID NO: 75 - RNA, 82 nt, Homo sapiens
ggccugcccgacacucuuucccuguugcacuacuauaggccgcugggaagcagugcaaugaugaaagggcaucggucagguc

SEQ ID NO: 76 - RNA, 101 nt, Homo sapiens
ccgcccccgcgucuccagggcaaccguggcuuucgauuguuacugugggaacuggagguaacagucuacagccauggucgccccgcagcacgcccacgcgc

SEQ ID NO: 77 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 78 - RNA, 22 nt, Homo sapiens
uuugguccccuucaaccagcua

SEQ ID NO: 79 - RNA, 73 nt, Homo sapiens
cagggugugugacugguugaccagaggggcaugcacuguguucacccugugggccaccuagucaccaacccuc

SEQ ID NO: 80 - RNA, 90 nt, Homo sapiens
aggccucgcuguucucuauggcuuuuuauuccuaugugauucuacugcucacucauauagggauuggagccguggcgcacggcggggaca

SEQ ID NO: 81 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 82 - RNA, 82 nt, Homo sapiens
ugagcccucggaggacuccauuuguuuugaugauggauucuuaugcuccaucaucgucucaaaugagucuucagaggguucu

SEQ ID NO: 83 - RNA, 23 nt, Homo sapiens
uggagacgcggcccuguuggagu

SEQ ID NO: 84 - RNA, 23 nt, Homo sapiens
ucuacagugcacgugucuccagu

SEQ ID NO: 85 - RNA, 21 nt, Homo sapiens
uaccacaggguagaaccacgg

SEQ ID NO: 86 - RNA, 22 nt, Homo sapiens
cagugguuuuacccuaugguag

SEQ ID NO: 87 - RNA, 95 nt, Homo sapiens
cggccggcccuggguccaucuuccaguacaguguuggauggucuaauugugaagcuccuaacacugucugguaaagauggcucccggguggguuc

SEQ ID NO: 88 - RNA, 23 nt, Homo sapiens
uguaguguuuccuacuuuaugga

SEQ ID NO: 89 - RNA, 21 nt, Homo sapiens
cauaaaguagaaagcacuacu

SEQ ID NO: 90 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 91 - RNA, 86 nt, Homo sapiens
uggggcccuggcugggauaucaucauauacuguaaguuugcgaugagacacuacaguauagaugauguacuaguccgggcaccccc

SEQ ID NO: 92 - RNA, 88 nt, Homo sapiens
caccuuguccucacgguccaguuuucccaggaaucccuuagaugcuaagauggggauuccuggaaauacuguucuugaggucaugguu

SEQ ID NO: 93 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 94 - RNA, 22 nt, Homo sapiens
ugcccuguggacucaguucugg

SEQ ID NO: 95 - RNA, 22 nt, Homo sapiens
ugagaacugaauuccauaggcu

SEQ ID NO: 96 - RNA, 22 nt, Homo sapiens
gugugcggaaaugcuucugcua

SEQ ID NO: 97 - RNA, 68 nt, Homo sapiens
gaggcaaaguucugagacacuccgacucugaguaugauagaagucagugcacuacagaacuuugucuc

SEQ ID NO: 98 - RNA, 99 nt, Homo sapiens
caagcacgauuagcauuugaggugaaguucuguuauacacucaggcuguggcucucugaaagucagugcaucacagaacuuugucucgaaagcuuucua

SEQ ID NO: 99 - RNA, 89 nt, Homo sapiens
gccggcgcccgagcucuggcuccgugucuucacucccgugcuuguccgaggagggagggagggacgggggcugugcuggggcagcugga

SEQ ID NO: 100 - RNA, 84 nt, Homo sapiens
cuccccauggcccugucucccaacccuuguaccagugcugggcucagacccugguacaggccugggggacagggaccuggggac

SEQ ID NO: 101 - RNA, 21 nt, Homo sapiens
cuagacugaagcuccuugagg

SEQ ID NO: 102 - RNA, 21 nt, Homo sapiens
ucgaggagcucacagucuagu

SEQ ID NO: 103 - RNA, 87 nt, Homo sapiens
ugucccccccggcccagguucugugauacacuccgacucgggcucuggagcagucagugcaugacagaacuugggcccggaaggacc

SEQ ID NO: 104 - RNA, 84 nt, Homo sapiens
gugguacuugaagauagguuauccguguugccuucgcuuuauuugugacgaaucauacacgguugaccuauuuuucaguaccaa

SEQ ID NO: 105 - RNA, 65 nt, Homo sapiens
cuguuaaugcuaaucgugauagggguuuuugccuccaacugacuccuacauauuagcauuaacag

SEQ ID NO: 106 - RNA, 83 nt, Homo sapiens
ccuuggaguaaaguagcagcacauaaugguuuguggauuuugaaaaggugcaggccauauugugcugccucaaaaauacaagg

SEQ ID NO: 107 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 108 - RNA, 89 nt, Homo sapiens
gucagcagugccuuagcagcacguaaauauuggcguuaagauucuaaaauuaucuccaguauuaacugugcugcugaaguaagguugac

SEQ ID NO: 109 - RNA, 84 nt, Homo sapiens
gucagaauaaugucaaagugcuuacagugcagguagugauaugugcaucuacugcagugaaggcacuuguagcauuauggugac

SEQ ID NO: 110 - RNA, 110 nt, Homo sapiens
ugaguuuugagguugcuucagugaacauucaacgcugucggugaguuuggaauuaaaaucaaaaccaucgaccguugauuguacccuauggcuaaccaucaucuacucca

SEQ ID NO: 111 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 112 - RNA, 110 nt, Homo sapiens
cggaaaauuugccaaggguuugggggaacauucaaccugucggugaguuugggcagcucaggcaaaccaucgaccguugaguggacccugaggccuggaauugccauccu

SEQ ID NO: 113 - RNA, 110 nt, Homo sapiens
gagcugcuugccuccccccguuuuuggcaaugguagaacucacacuggugagguaacaggauccggugguucuagacuugccaacuauggggcgaggacucagccggcac

SEQ ID NO: 114 - RNA, 18 nt, Homo sapiens
uccagugcccuccucucc

SEQ ID NO: 115 - RNA, 110 nt, Homo sapiens
ccgcagagugugacuccuguucuguguauggcacugguagaauucacugugaacagucucagucagugaauuaccgaagggccauaaacagagcagagacagauccacga

SEQ ID NO: 116 - RNA, 22 nt, Homo sapiens
uggacggagaacugauaagggu

SEQ ID NO: 117 - RNA, 82 nt, Homo sapiens
agggggcgagggauuggagagaaaggcaguuccugaugguccccuccccaggggcuggcuuuccucugguccuucccuccca

SEQ ID NO: 118 - RNA, 86 nt, Homo sapiens
ugcuuguaacuuuccaaagaauucuccuuuugggcuuucugguuuuauuuuaagcccaaaggugaauuuuuugggaaguuugagcu

SEQ ID NO: 119 - RNA, 109 nt, Homo sapiens
ggucgggcucaccaugacacagugugagaccucgggcuacaacacaggacccgggcgcugcucugaccccucgugucuuguguugcagccggagggacgcagguccgca

SEQ ID NO: 120 - RNA, 21 nt, Homo sapiens
cucccacaugcaggguuugca

SEQ ID NO: 121 - RNA, 22 nt, Homo sapiens
ugccuacugagcugauaucagu

SEQ ID NO: 122 - RNA, 71 nt, Homo sapiens
uguucuaaggugcaucuagugcagauagugaaguagauuagcaucuacugcccuaagugcuccuucuggca

SEQ ID NO: 123 - RNA, 71 nt, Homo sapiens
uguguuaaggugcaucuagugcaguuagugaagcagcuuagaaucuacugcccuaaaugccccuucuggca

SEQ ID NO: 124 - RNA, 85 nt, Homo sapiens
ugcaggccucugugugauauguuugauauauuagguuguuauuuaauccaacuauauaucaaacauauuccuacagugucuugcc

SEQ ID NO: 125 - RNA, 21 nt, Homo sapiens
ugauauguuugauauuggguu

SEQ ID NO: 126 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 127 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 128 - RNA, 22 nt, Homo sapiens
aacuggccuacaaagucccagu

SEQ ID NO: 129 - RNA, 22 nt, Homo sapiens
ugggucuuugcgggcgagauga

SEQ ID NO: 130 - RNA, 83 nt, Homo sapiens
guggucucagaaucgggguuuugagggcgagaugaguuuauguuuuauccaacuggcccucaaagucccgcuuuuggggucau

SEQ ID NO: 131 - RNA, 85 nt, Homo sapiens
augguguuaucaaguguaacagcaacuccauguggacuguguaccaauuuccaguggagaugcuguuacuuuugaugguuaccaa

SEQ ID NO: 132 - RNA, 87 nt, Homo sapiens
agcuucccuggcucuagcagcacagaaauauuggcacagggaagcgagucugccaauauuggcugugcugcuccaggcaggguggug

SEQ ID NO: 133 - RNA, 84 nt, Homo sapiens
acuggucggugauuuagguaguuuccuguuguugggauccaccuuucucucgacagcacgacacugccuucauuacuucaguug

SEQ ID NO: 134 - RNA, 75 nt, Homo sapiens
ggcugugccggguagagagggcagugggagguaagagcucuucacccuucaccaccuucuccacccagcauggcc

SEQ ID NO: 135 - RNA, 22 nt, Homo sapiens
acaguagucugcacauugguua

SEQ ID NO: 136 - RNA, 23 nt, Homo sapiens
cccaguguucagacuaccuguuc

SEQ ID NO: 137 - RNA, 23 nt, Homo sapiens
cccaguguuuagacuaucuguuc

SEQ ID NO: 138 - RNA, 82 nt, Homo sapiens
gcaguccucuguuaguuuugcauaguugcacuacaagaagaauguaguugugcaaaucuaugcaaaacugaugguggccugc

SEQ ID NO: 139 - RNA, 87 nt, Homo sapiens
cacuguucuaugguuaguuuugcagguuugcauccagcugugugauauucugcugugcaaauccaugcaaaacugacugugguagug

SEQ ID NO: 140 - RNA, 90 nt, Homo sapiens
ccgggccccugugagcaucuuaccggacagugcuggauuucccagcuugacucuaacacugucugguaacgauguucaaaggugacccgc

SEQ ID NO: 141 - RNA, 95 nt, Homo sapiens
ccagcucgggcagccguggccaucuuacugggcagcauuggauggagucaggucucuaauacugccugguaaugaugacggcggagcccugcacg

SEQ ID NO: 142 - RNA, 68 nt, Homo sapiens
cccucgucuuacccagcaguguuugggugcgguugggagucucuaauacugccggguaaugauggagg

SEQ ID NO: 143 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 144 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 145 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 146 - RNA, 22 nt, Homo sapiens
uggaauguaaggaagugugugg

SEQ ID NO: 147 - RNA, 71 nt, Homo sapiens
guagcacuaaagugcuuauagugcagguaguguuuaguuaucuacugcauuaugagcacuuaaaguacugc

SEQ ID NO: 148 - RNA, 69 nt, Homo sapiens
aguaccaaagugcucauagugcagguaguuuuggcaugacucuacuguaguaugggcacuuccaguacu

SEQ ID NO: 149 - RNA, 72 nt, Homo sapiens
ugucggguagcuuaucagacugauguugacuguugaaucucauggcaacaccagucgaugggcugucugaca

SEQ ID NO: 150 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 151 - RNA, 110 nt, Homo sapiens
ucaccuggccaugugacuugugggcuucccuuugucauccuucgccuagggcucugagcagggcagggacagcaaaggggugcucaguugucacuucccacagcacggag

SEQ ID NO: 152 - RNA, 110 nt, Homo sapiens
cggggcaccccgcccggacagcgcgccggcaccuuggcucuagacugcuuacugcccgggccgcccucaguaacagucuccagucacggccaccgacgccuggccccgcc

SEQ ID NO: 153 - RNA, 110 nt, Homo sapiens; identical to SEQ ID NO: 110

SEQ ID NO: 154 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 155 - RNA, 110 nt, Homo sapiens
aucauucagaaaugguauacaggaaaaugaccuaugaauugacagacaauauagcugaguuugucugucauuucuuuaggccaauauucuguaugacugugcuacuucaa

SEQ ID NO: 156 - RNA, 110 nt, Homo sapiens
gugauaauguagcgagauuuucuguugugcuugaucuaaccaugugguugcgagguaugaguaaaacaugguuccgucaagcaccauggaacgucacgcagcuuucuaca

SEQ ID NO: 157 - RNA, 21 nt, Homo sapiens
ugauuguccaaacgcaauucu

SEQ ID NO: 158 - RNA, 85 nt, Homo sapiens
ggcugagccgcaguaguucuucaguggcaagcuuuauguccugacccagcuaaagcugccaguugaagaacuguugcccucugcc

SEQ ID NO: 159 - RNA, 21 nt, Homo sapiens
ccacaccguaucugacacuuu

SEQ ID NO: 160 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 161 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 162 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 163 - RNA, 81 nt, Homo sapiens
gggcuuucaagucacuagugguuccguuuaguagaugauugugcauuguuucaaaauggugcccuagugacuacaaagccc

SEQ ID NO: 164 - RNA, 73 nt, Homo sapiens
ggccggcugggguuccuggggaugggauuugcuuccugucacaaaucacauugccagggauuuccaaccgacc

SEQ ID NO: 165 - RNA, 97 nt, Homo sapiens
cucaggugcucuggcugcuuggguuccuggcaugcugauuugugacuuaagauuaaaaucacauugccagggauuaccacgcaaccacgaccuuggc

SEQ ID NO: 166 - RNA, 68 nt, Homo sapiens
cuccggugccuacugagcugauaucaguucucauuuuacacacuggcucaguucagcaggaacaggag

SEQ ID NO: 167 - RNA, 73 nt, Homo sapiens
cucugccucccgugccuacugagcugaaacacaguugguuuguguacacuggcucaguucagcaggaacaggg

SEQ ID NO: 168 - RNA, 84 nt, Homo sapiens
ggccaguguugagaggcggagacuugggcaauugcuggacgcugcccugggcauugcacuugucucggucugacagugccggcc

SEQ ID NO: 169 - RNA, 77 nt, Homo sapiens
guggccucguucaaguaauccaggauaggcugugcaggucccaaugggccuauucuugguuacuugcacggggacgc

SEQ ID NO: 170 - RNA, 84 nt, Homo sapiens
ggcuguggcuggauucaaguaauccaggauaggcuguuuccaucugugaggccuauucuugauuacuuguuucuggaggcagcu

SEQ ID NO: 171 - RNA, 77 nt, Homo sapiens
ccgggacccaguucaaguaauucaggauagguugugugcuguccagccuguucuccauuacuuggcucggggaccgg

SEQ ID NO: 172 - RNA, 78 nt, Homo sapiens
cugaggagcagggcuuagcugcuugugagcaggguccacaccaagucguguucacaguggcuaaguuccgccccccag

SEQ ID NO: 173 - RNA, 97 nt, Homo sapiens
accucucuaacaaggugcagagcuuagcugauuggugaacagugauugguuuccgcuuuguucacaguggcuaaguucugcaccugaagagaaggug

SEQ ID NO: 174 - RNA, 22 nt, Homo sapiens
cacuagauugugagcuccugga

SEQ ID NO: 175 - RNA, 22 nt, Homo sapiens
aaggagcucacagucuauugag

SEQ ID NO: 176 - RNA, 21 nt, Homo sapiens
agggcccccccucaauccugu

SEQ ID NO: 177 - RNA, 22 nt, Homo sapiens
uaugugggaugguaaaccgcuu

SEQ ID NO: 178 - RNA, 22 nt, Homo sapiens
ugguuuaccgucccacauacau

SEQ ID NO: 179 - RNA, 64 nt, Homo sapiens
augacugauuucuuuugguguucagagucaauauaauuuucuagcaccaucugaaaucgguuau

SEQ ID NO: 180 - RNA, 81 nt, Homo sapiens
cuucaggaagcugguuucauauggugguuuagauuuaaauagugauugucuagcaccauuugaaaucaguguucuuggggg

SEQ ID NO: 181 - RNA, 81 nt, Homo sapiens
cuucuggaagcugguuucacaugguggcuuagauuuuuccaucuuuguaucuagcaccauuugaaaucaguguuuuaggag

SEQ ID NO: 182 - RNA, 88 nt, Homo sapiens
aucucuuacacaggcugaccgauuucuccugguguucagagucuguuuuugucuagcaccauuugaaaucgguuaugauguaggggga

SEQ ID NO: 183 - RNA, 86 nt, Homo sapiens
acugcuaacgaaugcucugacuuuauugcacuacuguacuuuacagcuagcagugcaauaguauugucaaagcaucugaaagcagg

SEQ ID NO: 184 - RNA, 23 nt, Homo sapiens
cagugcaaugauauugucaaagc

SEQ ID NO: 185 - RNA, 69 nt, Homo sapiens
ccaccacuuaaacguggauguacuugcuuugaaacuaaagaaguaagugcuuccauguuuuggugaugg

SEQ ID NO: 186 - RNA, 73 nt, Homo sapiens
gcucccuucaacuuuaacauggaagugcuuucugugacuuuaaaaguaagugcuuccauguuuuaguaggagu

SEQ ID NO: 187 - RNA, 68 nt, Homo sapiens
ccuuugcuuuaacauggggguaccugcugugugaaacaaaaguaagugcuuccauguuucaguggagg

SEQ ID NO: 188 - RNA, 68 nt, Homo sapiens
ccucuacuuuaacauggaggcacuugcugugacaugacaaaaauaagugcuuccauguuugagugugg

SEQ ID NO: 189 - RNA, 22 nt, Homo sapiens
cuuucagucggauguuugcagc

SEQ ID NO: 190 - RNA, 22 nt, Homo sapiens
uguaaacauccucgacuggaag

SEQ ID NO: 191 - RNA, 88 nt, Homo sapiens
accaaguuucaguucauguaaacauccuacacucagcuguaauacauggauuggcugggagguggauguuuacuucagcugacuugga

SEQ ID NO: 192 - RNA, 89 nt, Homo sapiens
accaugcuguaguguguguaaacauccuacacucucagcugugagcucaagguggcugggagaggguuguuuacuccuucugccaugga

SEQ ID NO: 193 - RNA, 72 nt, Homo sapiens
agauacuguaaacauccuacacucucagcuguggaaaguaagaaagcugggagaaggcuguuuacucuuucu

SEQ ID NO: 194 - RNA, 70 nt, Homo sapiens
guuguuguaaacauccccgacuggaagcuguaagacacagcuaagcuuucagucagauguuugcugcuac

SEQ ID NO: 195 - RNA, 22 nt, Homo sapiens
cuuucagucggauguuuacagc

SEQ ID NO: 196 - RNA, 71 nt, Homo sapiens
ggagaggaggcaagaugcuggcauagcuguugaacugggaaccugcuaugccaacauauugccaucuuucc

SEQ ID NO: 197 - RNA, 70 nt, Homo sapiens
ggagauauugcacauuacuaaguugcauguugucacggccucaaugcaauuuagugugugugauauuuuc

SEQ ID NO: 198 - RNA, 22 nt, Homo sapiens
aaaagcuggguugagagggcga

SEQ ID NO: 199 - RNA, 22 nt, Homo sapiens
aaaagcuggguugagagggcaa

SEQ ID NO: 200 - RNA, 21 nt, Homo sapiens
cacauuacacggucgaccucu

SEQ ID NO: 201 - RNA, 20 nt, Homo sapiens
acugccccaggugcugcugg

SEQ ID NO: 202 - RNA, 23 nt, Homo sapiens
cgcauccccuagggcauuggugu

SEQ ID NO: 203 - RNA, 23 nt, Homo sapiens
ccuaguagguguccaguaagugu

SEQ ID NO: 204 - RNA, 75 nt, Homo sapiens
uggagugggggggcaggaggggcucagggagaaagugcauacagccccuggcccucucugcccuuccguccccug

SEQ ID NO: 205 - RNA, 80 nt, Homo sapiens
gguaccugaagagagguuuucuggguuucuguuucuuuaaugaggacgaaacacaccugguuaaccucuuuuccaguauc

SEQ ID NO: 206 - RNA, 23 nt, Homo sapiens
gcaaagcacacggccugcagaga

SEQ ID NO: 207 - RNA, 21 nt, Homo sapiens
gccccugggccuauccuagaa

SEQ ID NO: 208 - RNA, 22 nt, Homo sapiens
cuagguauggucccagggaucc

SEQ ID NO: 209 - RNA, 94 nt, Homo sapiens
uguuuugagcgggggucaagagcaauaacgaaaaauguuugucauaaaccguuuuucauuauugcuccugaccuccucucauuugcuauauuca

SEQ ID NO: 210 - RNA, 21 nt, Homo sapiens
gaacggcuucauacaggaguu

SEQ ID NO: 211 - RNA, 22 nt, Homo sapiens
uccagcaucagugauuuuguug

SEQ ID NO: 212 - RNA, 22 nt, Homo sapiens
aacaauauccuggugcugagug

SEQ ID NO: 213 - RNA, 23 nt, Homo sapiens
ugagcgccucgacgacagagccg

SEQ ID NO: 214 - RNA, 23 nt, Homo sapiens
ucccuguccuccaggagcucacg

SEQ ID NO: 215 - RNA, 69 nt, Homo sapiens
cuguggugcauuguaguugcauugcauguucuggugguacccaugcaauguuuccacagugcaucacag

SEQ ID NO: 216 - RNA, 96 nt, Homo sapiens
gcgggcggccccgcggugcauugcuguugcauugcacgugugugaggcgggugcagugccucggcagugcagcccggagccggccccuggcaccac

SEQ ID NO: 217 - RNA, 95 nt, Homo sapiens
uuguaccuggugugauuauaaagcaaugagacugauugucauaugucguuugugggauccgucucaguuacuuuauagccauaccugguaucuua

SEQ ID NO: 218 - RNA, 23 nt, Homo sapiens
ucucacacagaaaucgcacccgu

SEQ ID NO: 219 - RNA, 21 nt, Homo sapiens
aggggugcuaucugugauuga

SEQ ID NO: 220 - RNA, 98 nt, Homo sapiens
acccaaacccuaggucugcugacuccuaguccagggcucgugauggcuggugggcccugaacgaggggucuggaggccuggguuugaauaucgacagc

SEQ ID NO: 221 - RNA, 23 nt, Homo sapiens
ugucugcccgcaugccugccucu

SEQ ID NO: 222 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 223 - RNA, 84 nt, Homo sapiens
gugcucgguuuguaggcagugucauuagcugauuguacuguggugguuacaaucacuaacuccacugccaucaaaacaaggcac

SEQ ID NO: 224 - RNA, 23 nt, Homo sapiens
ucccccaggugugauucugauuu

SEQ ID NO: 225 - RNA, 22 nt, Homo sapiens
uuaucagaaucuccagggguac

SEQ ID NO: 226 - RNA, 24 nt, Homo sapiens
aauccuuggaaccuaggugugagu

SEQ ID NO: 227 - RNA, 75 nt, Homo sapiens
uguugucggguggaucacgaugcaauuuugaugaguaucauaggagaaaaauugcacgguauccaucuguaaacc

SEQ ID NO: 228 - RNA, 87 nt, Homo sapiens
accgcagggaaaaugagggacuuuugggggcagauguguuuccauuccacuaucauaaugccccuaaaaauccuuauugcucuugca

SEQ ID NO: 229 - RNA, 68 nt, Homo sapiens
ccauuacuguugcuaauaugcaacucuguugaauauaaauuggaauugcacuuuagcaauggugaugg

SEQ ID NO: 230 - RNA, 21 nt, Homo sapiens
aauaauacaugguugaucuuu

SEQ ID NO: 231 - RNA, 75 nt, Homo sapiens
agacagagaagccaggucacgucucugcaguuacacagcucacgagugccugcugggguggaaccuggucugucu

SEQ ID NO: 232 - RNA, 67 nt, Homo sapiens
gugggccucaaauguggagcacuauucugauguccaaguggaaagugcugcgacauuugagcgucac

SEQ ID NO: 233 - RNA, 69 nt, Homo sapiens
gggauacucaaaaugggggcgcuuuccuuuuugucuguacugggaagugcuucgauuuugggguguccc

SEQ ID NO: 234 - RNA, 72 nt, Homo sapiens
uacaucggccauuauaauacaaccugauaaguguuauagcacuuaucagauuguauuguaauugucugugua

SEQ ID NO: 235 - RNA, 72 nt, Homo sapiens
acucggauggauauaauacaaccugcuaaguguccuagcacuuagcagguuguauuaucauuguccgugucu

SEQ ID NO: 236 - RNA, 22 nt, Homo sapiens
uuuguucguucggcucgcguga

SEQ ID NO: 237 - RNA, 68 nt, Homo sapiens
uaaaagguagauucuccuucuaugaguacauuauuuaugauuaaucauagaggaaaauccacguuuuc

SEQ ID NO: 238 - RNA, 100 nt, Homo sapiens
caguccuucuuugguauuuaaaacguggauauuccuucuauguuuacgugauuccugguuaaucauagaggaaaauccauguuuucaguaucaaaugcug

SEQ ID NO: 239 - RNA, 66 nt, Homo sapiens
aaaagguggauauuccuucuauguuuauguuauuuaugguuaaacauagaggaaauuccacguuuu

SEQ ID NO: 240 - RNA, 66 nt, Homo sapiens
agggcuccugacuccagguccuguguguuaccuagaaauagcacuggacuuggagucagaaggccu

SEQ ID NO: 241 - RNA, 67 nt, Homo sapiens
agagaugguagacuauggaacguaggcguuaugauuucugaccuauguaacaugguccacuaacucu

SEQ ID NO: 242 - RNA, 61 nt, Homo sapiens
aagaugguugaccauagaacaugcgcuaucucugugucguauguaauaugguccacaucuu

SEQ ID NO: 243 - RNA, 75 nt, Homo sapiens
uacuuaaagcgagguugcccuuuguauauucgguuuauugacauggaauauacaagggcaagcucucugugagua

SEQ ID NO: 244 - RNA, 76 nt, Homo sapiens
uacuugaagagaaguuguucgugguggauucgcuuuacuuaugacgaaucauucacggacaacacuuuuuucagua

SEQ ID NO: 245 - RNA, 20 nt, Homo sapiens
auuccuagaaauuguucaua

SEQ ID NO: 246 - RNA, 22 nt, Homo sapiens
gaauguugcucggugaaccccu

SEQ ID NO: 247 - RNA, 23 nt, Homo sapiens
agguuacccgagcaacuuugcau

SEQ ID NO: 248 - RNA, 80 nt, Homo sapiens
gguaccugagaagagguugucugugaugaguucgcuuuuauuaaugacgaauauaacacagauggccuguuuucaguacc

SEQ ID NO: 249 - RNA, 96 nt, Homo sapiens
ugguacuuggagagauaguagaccguauagcguacgcuuuaucugugacguauguaacacgguccacuaacccucaguaucaaauccauccccgag

SEQ ID NO: 250 - RNA, 23 nt, Homo sapiens
aucaacagacauuaauugggcgc

SEQ ID NO: 251 - RNA, 22 nt, Homo sapiens
acuggacuuagggucagaaggc

SEQ ID NO: 252 - RNA, 23 nt, Homo sapiens
agcucggucugaggccccucagu

SEQ ID NO: 253 - RNA, 23 nt, Homo sapiens
ugaggggcagagagcgagacuuu

SEQ ID NO: 254 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 255 - RNA, 87 nt, Homo sapiens
gaaagcgcuuuggaaugacacgaucacucccguugagugggcacccgagaagccaucgggaaugucguguccgcccagugcucuuuc

SEQ ID NO: 256 - RNA, 22 nt, Homo sapiens
uaauacugucugguaaaaccgu

SEQ ID NO: 257 - RNA, 114 nt, Homo sapiens
uccugcuuguccugcgaggugucuugcaggccgucaugcaggccacacugacgguaacguugcaggucgucuugcagggcuucucgcaagacgacauccucaucaccaacgacg

SEQ ID NO: 258 - RNA, 94 nt, Homo sapiens
ugacuccuccaggucuuggaguaggucauuggguggauccucuauuuccuuacgugggccacuggauggcuccuccaugucuuggaguagauca

SEQ ID NO: 259 - RNA, 93 nt, Homo sapiens
ccggggagaaguacggugagccugucauuauucagagaggcuagauccucuguguugagaaggaucaugaugggcuccucgguguucuccagg

SEQ ID NO: 260 - RNA, 22 nt, Homo sapiens
uggcaguguauuguuagcuggu

SEQ ID NO: 261 - RNA, 91 nt, Homo sapiens
aaacgauacuaaacuguuuuugcgauguguuccuaauaugcacuauaaauauauugggaacauuuugcauguauaguuuuguaucaauaua

SEQ ID NO: 262 - RNA, 22 nt, Homo sapiens
uugggaucauuuugcauccaua

SEQ ID NO: 263 - RNA, 22 nt, Homo sapiens
uuuugcaauauguuccugaaua

SEQ ID NO: 264 - RNA, 22 nt, Homo sapiens
aaaccguuaccauuacugaguu

SEQ ID NO: 265 - RNA, 85 nt, Homo sapiens
gcuaagcacuuacaacuguuugcagaggaaacugagacuuuguaacuaugucucagucucaucugcaaagaaguaagugcuuugc

SEQ ID NO: 266 - RNA, 115 nt, Homo sapiens
ucuguuuaucaccagauccuagaacccuaucaauauugucucugcuguguaaauaguucugaguagugcaauauugcuuauaggguuuugguguuuggaaagaacaaugggcagg

SEQ ID NO: 267 - RNA, 21 nt, Homo sapiens
gcaguccaugggcauauacac

SEQ ID NO: 268 - RNA, 21 nt, Homo sapiens
ucacuccucuccucccgucuu

SEQ ID NO: 269 - RNA, 22 nt, Homo sapiens
aagacgggaggaaagaagggag

SEQ ID NO: 270 - RNA, 22 nt, Homo sapiens
ucaggcucaguccccucccgau

SEQ ID NO: 271 - RNA, 22 nt, Homo sapiens
gucauacacggcucuccucucu

SEQ ID NO: 272 - RNA, 22 nt, Homo sapiens
agaggcuggccgugaugaauuc

SEQ ID NO: 273 - RNA, 21 nt, Homo sapiens
cggggcagcucaguacaggau

SEQ ID NO: 274 - RNA, 22 nt, Homo sapiens
uccuguacugagcugccccgag

SEQ ID NO: 275 - RNA, 80 nt, Homo sapiens
gguacuugaagagugguuaucccugcuguguucgcuuaauuuaugacgaaucauacagggacauccaguuuuucaguauc

SEQ ID NO: 276 - RNA, 84 nt, Homo sapiens
uugguacuuggagagugguuaucccuguccuguucguuuugcucaugucgaaucguacagggucauccacuuuuucaguaucaa

SEQ ID NO: 277 - RNA, 83 nt, Homo sapiens
gagaaucaucucucccagauaauggcacucucaaacaaguuuccaaauuguuugaaaggcuauuucuuggucagaugacucuc

SEQ ID NO: 278 - RNA, 84 nt, Homo sapiens
guggcagcuugguggucguaugugugacgccauuuacuugaaccuuuaggagugacaucacauauacggcagcuaaacugcuac

SEQ ID NO: 279 - RNA, 22 nt, Homo sapiens
caaccuggaggacuccaugcug

SEQ ID NO: 280 - RNA, 22 nt, Homo sapiens
aguggggaacccuuccaugagg

SEQ ID NO: 281 - RNA, 23 nt, Homo sapiens
aggaccugcgggacaagauucuu

SEQ ID NO: 282 - RNA, 89 nt, Homo sapiens
cuggccuccagggcuuuguacaugguaggcuuucauucauucguuugcacauucggugaaggucuacugugugccaggcccugugccag

SEQ ID NO: 283 - RNA, 22 nt, Homo sapiens
ugaaggucuacugugugccagg

SEQ ID NO: 284 - RNA, 81 nt, Homo sapiens
gauacucgaaggagagguuguccguguugucuucucuuuauuuaugaugaaacauacacgggaaaccucuuuuuuaguauc

SEQ ID NO: 285 - RNA, 82 nt, Homo sapiens
ugguaccugaaaagaaguugcccauguuauuuucgcuuuauaugugacgaaacaaacauggugcacuucuuuuucgguauca

SEQ ID NO: 286 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 287 - RNA, 22 nt, Homo sapiens
aacaucacagcaagucugugcu

SEQ ID NO: 288 - RNA, 21 nt, Homo sapiens
uuaagacuugcagugauguuu

SEQ ID NO: 289 - RNA, 84 nt, Homo sapiens
gcucccccucucuaauccuugcuaccugggugagagugcugucugaaugcaaugcaccugggcaaggauucugagagcgagagc

SEQ ID NO: 290 - RNA, 22 nt, Homo sapiens
aauccuuugucccugggugaga

SEQ ID NO: 291 - RNA, 22 nt, Homo sapiens
aaugcaccugggcaaggauuca

SEQ ID NO: 292 - RNA, 21 nt, Homo sapiens
auccuugcuaucugggugcua

SEQ ID NO: 293 - RNA, 84 nt, Homo sapiens
gaugcacccagugggggagccaggaaguauugauguuucugccaguuuagcgucaacacuugcugguuuccucucuggagcauc

SEQ ID NO: 294 - RNA, 124 nt, Homo sapiens
gccaccaccaucagccauacuauguguagugccuuauucaggaagguguuacuuaauagauuaauauuuguaaggcacccuucugaguagaguaaugugcaacauggacaacauuugugguggc

SEQ ID NO: 295 - RNA, 21 nt, Homo sapiens
uuuugcaccuuuuggagugaa

SEQ ID NO: 296 - RNA, 23 nt, Homo sapiens
ugauuguagccuuuuggaguaga

SEQ ID NO: 297 - RNA, 21 nt, Homo sapiens
uacugcagacaguggcaauca

SEQ ID NO: 298 - RNA, 87 nt, Homo sapiens
caauagacacccaucgugucuuuugcucugcagucaguaaauauuuuuuugugaauguguagcaaaagacagaauggugguccauug

SEQ ID NO: 299 - RNA, 22 nt, Homo sapiens
aagugcugucauagcugagguc

SEQ ID NO: 300 - RNA, 84 nt, Homo sapiens
gcguacagugccuuucucaaggaggugucguuuaugugaacuaaaauauaaauuucaccuuucugagaagaguaauguacagca

SEQ ID NO: 301 - RNA, 22 nt, Homo sapiens
gagugccuucuuuuggagcguu

SEQ ID NO: 302 - RNA, 18 nt, Homo sapiens
ugcuuccuuucagagggu

SEQ ID NO: 303 - RNA, 87 nt, Homo sapiens
ucucaggcagugacccucuagauggaagcacugucuguuguauaaaagaaaagaucgugcaucccuuuagaguguuacuguuugaga

SEQ ID NO: 304 - RNA, 67 nt, Homo sapiens
gugacccucuagauggaagcacugucuguugucuaagaaaagaucgugcaucccuuuagaguguuac

SEQ ID NO: 305 - RNA, 95 nt, Homo sapiens
gaagaucucaggcagugacccucuagauggaagcacugucuguugucuaagaaaagaucgugcauccuuuuagaguguuacuguuugagaaaauc

SEQ ID NO: 306 - RNA, 22 nt, Homo sapiens
caaagcgcuccccuuuagaggu

SEQ ID NO: 307 - RNA, 21 nt, Homo sapiens
caaagcgcuucccuuuggagc

SEQ ID NO: 308 - RNA, 22 nt, Homo sapiens
cucuagagggaagcacuuucug

SEQ ID NO: 309 - RNA, 88 nt, Homo sapiens
ucucaggcugugacccucuagagggaagcgcuuucuguuggcuaaaagaaaagaaagcgcuucccuucagaguguuaacgcuuugaga

SEQ ID NO: 310 - RNA, 87 nt, Homo sapiens
ucucaugcugugacccucuagagggaagcacuuucucuugucuaaaagaaaagaaagcgcuucucuuuagaggauuacucuuugaga

SEQ ID NO: 311 - RNA, 85 nt, Homo sapiens
cucaggcugugacacucuagagggaagcgcuuucuguugucugaaagaaaggaaagugcauccuuuuagaguguuacuguuugag

SEQ ID NO: 312 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 313 - RNA, 84 nt, Homo sapiens
ucucaugcagucauucuccaaaagggagcacuuucuguuugaaagaaaacaaagugccuccuuuuagaguguuacuguuugaga

SEQ ID NO: 314 - RNA, 21 nt, Homo sapiens
cuccagagggaaguacuuucu

SEQ ID NO: 315 - RNA, 21 nt, Homo sapiens
aaagugcuuccuuuuagaggg

SEQ ID NO: 316 - RNA, 22 nt, Homo sapiens
aaagugcuuccuuuuagagggu

SEQ ID NO: 317 - RNA, 22 nt, Homo sapiens
aaagugcuucucuuuggugggu

SEQ ID NO: 318 - RNA, 20 nt, Homo sapiens
cuacaaagggaagcccuuuc

SEQ ID NO: 319 - RNA, 90 nt, Homo sapiens
ucccaugcugugacccucuagaggaagcacuuucuguuuguugucugagaaaaaacaaagugcuucccuuuagaguguuaccguuuggga

SEQ ID NO: 320 - RNA, 22 nt, Homo sapiens
aacgcacuucccuuuagagugu

SEQ ID NO: 321 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 322 - RNA, 87 nt, Homo sapiens
ucucaugcugugacccucuagagggaagcgcuuucuguugucugaaagaaaagaacgcgcuucccuauagaggguuacccuuugaga

SEQ ID NO: 323 - RNA, 22 nt, Homo sapiens
gaaggcgcuucccuuuagagcg

SEQ ID NO: 324 - RNA, 22 nt, Homo sapiens
ccucccacacccaaggcuugca

SEQ ID NO: 325 - RNA, 22 nt, Homo sapiens
caugccuugaguguaggaccgu

SEQ ID NO: 326 - RNA, 78 nt, Homo sapiens
auacuugaggagaaauuauccuugguguguucgcuuuauuuaugaugaaucauacaaggacaauuucuuuuugaguau

SEQ ID NO: 327 - RNA, 84 nt, Homo sapiens
acgucagggaaaggauucugcugucggucccacuccaaaguucacagaauggguggugggcacagaaucuggacucugcuugug

SEQ ID NO: 328 - RNA, 22 nt, Homo sapiens
aaacauucgcggugcacuucuu

SEQ ID NO: 329 - RNA, 106 nt, Homo sapiens
cccagccuggcacauuaguaggccucaguaaauguuuauuagaugaauaaaugaaugacucaucagcaaacauuuauugugugccugcuaaagugagcuccacagg

SEQ ID NO: 330 - RNA, 22 nt, Homo sapiens
caaaacuggcaauuacuuuugc

SEQ ID NO: 331 - RNA, 22 nt, Homo sapiens
aaaaguaauugcgaguuuuacc

SEQ ID NO: 332 - RNA, 22 nt, Homo sapiens
aaaaguaauugugguuuuggcc

SEQ ID NO: 333 - RNA, 22 nt, Homo sapiens
caaaaaucucaauuacuuuugc

SEQ ID NO: 334 - RNA, 102 nt, Homo sapiens
ucuguccauuaggugggugcaaaaguaaucgcgguuuuugucauuacuuuuaaugguaaaaacuggaauuacuuuugcacugaccuaauauuaagccagaua

SEQ ID NO: 335 - RNA, 22 nt, Homo sapiens
caaaaguaauuguggauuuugu

SEQ ID NO: 336 - RNA, 96 nt, Homo sapiens
agaugugcucuccuggcccaugaaaucaagcgugggugagaccuggugcagaacgggaaggcgacccauacuugguuucagaggcugugagaauaa

SEQ ID NO: 337 - RNA, 96 nt, Homo sapiens
aaccauucaaauauaccacaguuuguuuaaccuuuugccuguugguugaagaugccuuucaacaggugacugguuagacaaacugugguauauaca

SEQ ID NO: 338 - RNA, 21 nt, Homo sapiens
gcuaguccugacucagccagu

SEQ ID NO: 339 - RNA, 22 nt, Homo sapiens
gaugagcucauuguaauaugag

SEQ ID NO: 340 - RNA, 23 nt, Homo sapiens
guuugcacgggugggccuugucu

SEQ ID NO: 341 - RNA, 20 nt, Homo sapiens
aaaguagcuguaccauuugc

SEQ ID NO: 342 - RNA, 19 nt, Homo sapiens
aggcacggugucagcaggc

SEQ ID NO: 343 - RNA, 19 nt, Homo sapiens
gggcgccugugaucccaac

SEQ ID NO: 344 - RNA, 21 nt, Homo sapiens
aguuaaugaauccuggaaagu

SEQ ID NO: 345 - RNA, 97 nt, Homo sapiens
cuagauaaguuauuaggugggugcaaagguaauugcaguuuuucccauuauuuuaauugcgaaaacagcaauuaccuuugcaccaaccugauggagu

SEQ ID NO: 346 - RNA, 21 nt, Homo sapiens
ugaguuggccaucugagugag

SEQ ID NO: 347 - RNA, 20 nt, Homo sapiens
guccgcucggcgguggccca

SEQ ID NO: 348 - RNA, 22 nt, Homo sapiens
cacgcucaugcacacacccaca

SEQ ID NO: 349 - RNA, 22 nt, Homo sapiens
aagauguggaaaaauuggaauc

SEQ ID NO: 350 - RNA, 22 nt, Homo sapiens
auucuaauuucuccacgucuuu

SEQ ID NO: 351 - RNA, 21 nt, Homo sapiens
uagauaaaauauugguaccug

SEQ ID NO: 352 - RNA, 21 nt, Homo sapiens
cuucuugugcucuaggauugu

SEQ ID NO: 353 - RNA, 98 nt, Homo sapiens
cauauuagguuaaugcaaaaguaaucgcgguuugugccagaugacgauuugaauuaauaaauucauuugguauaaaccgcgauuauuuuugcaucaac

SEQ ID NO: 354 - RNA, 21 nt, Homo sapiens
ucuuguguucucuagaucagu

SEQ ID NO: 355 - RNA, 22 nt, Homo sapiens
uaacugguugaacaacugaacc

SEQ ID NO: 356 - RNA, 23 nt, Homo sapiens
uuacaguuguucaaccaguuacu

SEQ ID NO: 357 - RNA, 21 nt, Homo sapiens
caaagaggaaggucccauuac

SEQ ID NO: 358 - RNA, 97 nt, Homo sapiens
uagggugaccagccauuaugguuugccugggacugaggaauuugcugggauaugucaguuccaggccaaccaggcugguuggucucccugaagcaac

SEQ ID NO: 359 - RNA, 21 nt, Homo sapiens
uuuccauaggugaugagucac

SEQ ID NO: 360 - RNA, 21 nt, Homo sapiens
uuggccacaauggguuagaac

SEQ ID NO: 361 - RNA, 99 nt, Homo sapiens
uccagccugugcccagcagccccugagaaccacgucugcucugagcuggguacugccuguucagaacaaaugccgguucccagacgcugccagcuggcc

SEQ ID NO: 362 - RNA, 21 nt, Homo sapiens
uaauuuuauguauaagcuagu

SEQ ID NO: 363 - RNA, 22 nt, Homo sapiens
gagcuuauucauaaaagugcag

SEQ ID NO: 364 - RNA, 20 nt, Homo sapiens
agaccauggguucucauugu

SEQ ID NO: 365 - RNA, 21 nt, Homo sapiens
gaagugugccguggugugucu

SEQ ID NO: 366 - RNA, 97 nt, Homo sapiens
uacuuacucuacgugugugucacucgaugaccacugugaagacaguaaaauguacagugguucucuuguggcucaagcguaauguagaguacugguc

SEQ ID NO: 367 - RNA, 97 nt, Homo sapiens
gcuugaugaugcugcugaugcuggcggugaucccgauggugugagcuggaaauggggugcuacgucaucguugucaucgucaucaucaucauccgag

SEQ ID NO: 368 - RNA, 22 nt, Homo sapiens
uggucuaggauuguuggaggag

SEQ ID NO: 369 - RNA, 22 nt, Homo sapiens
cacacacugcaauuacuuuugc

SEQ ID NO: 370 - RNA, 19 nt, Homo sapiens
aggcugcggaauucaggac

SEQ ID NO: 371 - RNA, 21 nt, Homo sapiens
aaacuacugaaaaucaaagau

SEQ ID NO: 372 - RNA, 21 nt, Homo sapiens
guucaaauccagaucuauaac

SEQ ID NO: 373 - RNA, 20 nt, Homo sapiens
aggaauguuccuucuuugcc

SEQ ID NO: 374 - RNA, 23 nt, Homo sapiens
gaacgccuguucuugccaggugg

SEQ ID NO: 375 - RNA, 22 nt, Homo sapiens
uccgagccugggucucccucuu

SEQ ID NO: 376 - RNA, 22 nt, Homo sapiens
ggggguccccggugcucggauc

SEQ ID NO: 377 - RNA, 97 nt, Homo sapiens
uuagguaauuccuccacucaaaacccuucagugacuuccaugacaugaaauaggaagucauuggaggguuugagcagaggaaugaccuguuuuaaaa

SEQ ID NO: 378 - RNA, 22 nt, Homo sapiens
agacuucccauuugaagguggc

SEQ ID NO: 379 - RNA, 23 nt, Homo sapiens
aaacucuacuuguccuucugagu

SEQ ID NO: 380 - RNA, 97 nt, Homo sapiens
aaugcuguuucaagguaguaccaguaccuuguguucaguggaaccaagguaaacacaagguauugguauuaccuugagauagcauuacaccuaagug

SEQ ID NO: 381 - RNA, 85 nt, Homo sapiens
aggguagagggaugagggggaaaguucuauaguccuguaauuagaucucaggacuauagaacuuucccccucaucccucugcccu

SEQ ID NO: 382 - RNA, 19 nt, Homo sapiens
agcugucugaaaaugucuu

SEQ ID NO: 383 - RNA, 97 nt, Homo sapiens
uacuuauuacugguagugagucucuaagaaaagaggaggugguuguuuuccuccucuuuucuuugagacucacuaccaauaauaagaaauacuacua

SEQ ID NO: 384 - RNA, 21 nt, Homo sapiens
ucuaguaagaguggcagucga

SEQ ID NO: 385 - RNA, 22 nt, Homo sapiens
augcugacauauuuacuagagg

SEQ ID NO: 386 - RNA, 97 nt, Homo sapiens
ucccuuucccaggggaggggcuggguuuacguugggagaacuuuuacggugaaccaggagguucucccaacguaagcccagccccuccccucugccu

SEQ ID NO: 387 - RNA, 22 nt, Homo sapiens
aguauucuguaccagggaaggu

SEQ ID NO: 388 - RNA, 23 nt, Homo sapiens
cuaauaguaucuaccacaauaaa

SEQ ID NO: 389 - RNA, 23 nt, Homo sapiens
ugugcuugcucgucccgcccgca

SEQ ID NO: 390 - RNA, 24 nt, Homo sapiens
acugggggcuuucgggcucugcgu

SEQ ID NO: 391 - RNA, 25 nt, Homo sapiens
agggaucgcgggcggguggcggccu

SEQ ID NO: 392 - RNA, 23 nt, Homo sapiens
aucgcugcgguugcgagcgcugu

SEQ ID NO: 393 - RNA, 97 nt, Homo sapiens
aucugaguugggagggucccucuccaaaugugucuuggggugggggaucaagacacauuuggagagggaaccucccaacucggccucugccaucauu

SEQ ID NO: 394 - RNA, 19 nt, Homo sapiens
aguguggcuuucuuagagc

SEQ ID NO: 395 - RNA, 19 nt, Homo sapiens
ucuaggcugguacugcuga

SEQ ID NO: 396 - RNA, 19 nt, Homo sapiens
aagcagcugccucugaggc

SEQ ID NO: 397 - RNA, 21 nt, Homo sapiens
aggaggcagcgcucucaggac

SEQ ID NO: 398 - RNA, 98 nt, Homo sapiens
acgaauggcuaugcacugcacaacccuaggagagggugccauucacauagacuauaauugaauggcgccacuaggguugugcagugcacaaccuacac

SEQ ID NO: 399 - RNA, 22 nt, Homo sapiens
uaugucugcugaccaucaccuu

SEQ ID NO: 400 - RNA, 22 nt, Homo sapiens
uggugggccgcagaacaugugc

SEQ ID NO: 401 - RNA, 97 nt, Homo sapiens
aacuaugcaaggauauuugaggagagguuauccguguuauguucgcuucauucaucaugaauaauacaugguuaaccucuuuuugaauaucagacuc

SEQ ID NO: 402 - RNA, 78 nt, Homo sapiens
cugaaauagguugccugugagguguucacuuucuauaugaugaauauuauacagucaaccucuuuccgauaucgaauc

SEQ ID NO: 403 - RNA, 23 nt, Homo sapiens
ggcagguucucacccucucuagg

SEQ ID NO: 404 - RNA, 97 nt, Homo sapiens
cugcuccuucucccauacccauugcauaucggaguugugaauucucaaaacaccuccugugugcauggauuacaggagggugagccuugucaucgug

SEQ ID NO: 405 - RNA, 24 nt, Homo sapiens
ugccugggucucuggccugcgcgu

SEQ ID NO: 406 - RNA, 22 nt, Homo sapiens
gguggcccggccgugccugagg

SEQ ID NO: 407 - RNA, 82 nt, Homo sapiens
gaacauugaaacuggcuagggaaaaugauuggauagaaacuauuauucuauucauuuauccccagccuacaaaaugaaaaaa

SEQ ID NO: 408 - RNA, 66 nt, Homo sapiens
gguaagugcgccucgggugagcaugcacuuaauguggguguaugucacucggcucggcccacuacc

SEQ ID NO: 409 - RNA, 21 nt, Homo sapiens
uccgguucucagggcuccacc

SEQ ID NO: 410 - RNA, 23 nt, Homo sapiens
aggaagcccuggaggggcuggag

SEQ ID NO: 411 - RNA, 100 nt, Homo sapiens
gauggugaucuagcccuuuaguuuugagguugguguacugugugugaguauacauauuuaucacacacagucacuaucuucgaaagugagggugcacauc

SEQ ID NO: 412 - RNA, 22 nt, Homo sapiens
gcacugagaugggaguggugua

SEQ ID NO: 413 - RNA, 88 nt, Homo sapiens
aacugcccucaaggagcuuacaaucuagcuggggguaaaugacuugcacaugaacacaacuagacugugagcuucuagagggcaggga

SEQ ID NO: 414 - RNA, 17 nt, Homo sapiens
ucucgcuggggccucca

SEQ ID NO: 415 - RNA, 98 nt, Homo sapiens
uugggcaaggugcggggcuagggcuaacagcagucuuacugaagguuuccuggaaaccacgcacaugcuguugccacuaaccucaaccuuacucgguc

SEQ ID NO: 416 - RNA, 88 nt, Homo sapiens
gccuggauacaugagaugguugaccagagagcacacgcuuuauuugugccguuugugaccugguccacuaacccucaguaucuaaugc

SEQ ID NO: 417 - RNA, 21 nt, Homo sapiens
uggaggagaaggaaggugaug

SEQ ID NO: 418 - RNA, 111 nt, Homo sapiens
gcauccucaggaccugggcuugggugguaggaggaauuggugcuggucuuucauuuuggauuugacuccagccccacagccucagccaccccagccaauugucauaggagc

SEQ ID NO: 419 - RNA, 23 nt, Homo sapiens
ugcaccaugguugucugagcaug

SEQ ID NO: 420 - RNA, 22 nt, Homo sapiens
ugagaccucuggguucugagcu

SEQ ID NO: 421 - RNA, 23 nt, Homo sapiens
uccaguaccacgugucagggcca

SEQ ID NO: 422 - RNA, 21 nt, Homo sapiens
aagguuacuuguuaguucagg

SEQ ID NO: 423 - RNA, 78 nt, Homo sapiens
uuagcccugcggccccacgcaccaggguaagagagacucucgcuuccugcccuggcccgagggaccgacuggcugggc

SEQ ID NO: 424 - RNA, 22 nt, Homo sapiens
uauaccucaguuuuaucaggug

SEQ ID NO: 425 - RNA, 22 nt, Homo sapiens
uccauuacacuacccugccucu

SEQ ID NO: 426 - RNA, 21 nt, Homo sapiens
cgcgggugcuuacugacccuu

SEQ ID NO: 427 - RNA, 23 nt, Homo sapiens
cgggucggaguuagcucaagcgg

SEQ ID NO: 428 - RNA, 77 nt, Homo sapiens
ggcagugcucuacucaaaaagcugucagucacuuagauuacaugugacugacaccucuuugggugaaggaaggcuca

SEQ ID NO: 429 - RNA, 79 nt, Homo sapiens
gugcuuaaagaauggcuguccguaguauggucucuauauuuaugaugauuaauaucggacaaccauuguuuuaguaucc

SEQ ID NO: 430 - RNA, 21 nt, Homo sapiens
uacuuggaaaggcaucaguug

SEQ ID NO: 431 - RNA, 22 nt, Homo sapiens
cacuggcuccuuucuggguaga

SEQ ID NO: 432 - RNA, 89 nt, Homo sapiens
cgggguugguuguuaucuuugguuaucuagcuguaugagugguguggagucuucauaaagcuagauaaccgaaaguaaaaauaacccca

SEQ ID NO: 433 - RNA, 20 nt, Homo sapiens
agagucuugugaugucuugc

SEQ ID NO: 434 - RNA, 78 nt, Homo sapiens
cuuucuacacagguugggaucgguugcaaugcuguguuucuguaugguauugcacuugucccggccuguugaguuugg

SEQ ID NO: 435 - RNA, 80 nt, Homo sapiens
cugggggcuccaaagugcuguucgugcagguagugugauuacccaaccuacugcugagcuagcacuucccgagcccccgg

SEQ ID NO: 436 - RNA, 23 nt, Homo sapiens
ccaguuaccgcuuccgcuaccgc

SEQ ID NO: 437 - RNA, 86 nt, Homo sapiens
agcacugcccccggugagucaggguggggcuggcccccugcuucgugcccauccgcgcucugacucucugcccaccugcaggagcu

SEQ ID NO: 438 - RNA, 82 nt, Homo sapiens
ugugggcagggcccuggggagcugaggcucuggggguggccggggcugacccugggccucugcuccccagugucugaccgcg

SEQ ID NO: 439 - RNA, 86 nt, Homo sapiens
auuaggagaguaucuucucuguuuuggccauguguguacucacagccccucacacauggccgaaacagagaaguuacuuuccuaau

SEQ ID NO: 440 - RNA, 21 nt, Homo sapiens
cugacuguugccguccuccag

SEQ ID NO: 441 - RNA, 81 nt, Homo sapiens
aacacagugggcacucaauaaaugucuguugaauugaaaugcguuacauucaacggguauuuauugagcacccacucugug

SEQ ID NO: 442 - RNA, 78 nt, Homo sapiens
uggccgauuuuggcacuagcacauuuuugcuugugucucuccgcucugagcaaucaugugcagugccaauaugggaaa

SEQ ID NO: 443 - RNA, 119 nt, Homo sapiens
aggauucugcucaugccagggugagguaguaaguuguauuguugugggguagggauauuaggccccaauuagaagauaacuauacaacuuacuacuuucccugguguguggcauauuca

SEQ ID NO: 444 - RNA, 81 nt, Homo sapiens
cccauuggcauaaacccguagauccgaucuuguggugaaguggaccgcacaagcucgcuucuaugggucugugucagugug

SEQ ID NO: 445 - RNA, 70 nt, Homo sapiens
ggcacccacccguagaaccgaccuugcggggccuucgccgcacacaagcucgugucuguggguccguguc

SEQ ID NO: 446 - RNA, 550 nt, Artificial Sequence
  Synthetic
gugcucgcuucggcagcacauauacuaaaauuggaacgauacagagaagauuagcauggccccugcgcaaggaugacacgcaaauucgugaagcguuccauauuuuuacugcccuccaugcccugccccacaaacgcucugauaacagucugucccugucucucuccugcugcuccuauggaagcgaaguuuuccgcuccugcagaaagcaaaguuacgacucagagacggcugaggaugacaucagcgaugugcagggaacccagcgccuggagcuucgggaugacggggccuucagcacccccacgggggguucugacacccuggugggcaccucccuggacacacccccgaccuccgugacaggcaccucagaggagcaagugagcugguggggcagcgggcagacgguccuggagcaggaagcgggcaguggggguggcacccgccgccucccgggcagcccaaggcaagcacaggcaaccggggccgggccacggcaccuggggguggagccgcuggugcgggcaucucgagcuaaucugguggg

SEQ ID NO: 447 - RNA, 266 nt, Homo sapiens
gucuuugcaccaucucugaaaagccgauguguauccucagcuuugagaacugaauuccauggguugugucagugucagaccucugaaauucaguucuucagcugggauaucucugucaucgugggcuugaggaccuggagagaguagauccugaagaacuuuuucagucugcugaagagcuuggaagacuggagacagaaggcagagucucaggcucugaagguauaaggagugugaguuccugugagaaacacucauuugauugu

SEQ ID NO: 448 - RNA, 73 nt, Homo sapiens
ccuggcacugagaacugaauuccauaggcugugagcucuagcaaugcccuguggacucaguucuggugcccgg

SEQ ID NO: 449 - RNA, 73 nt, Homo sapiens; identical to SEQ ID NO: 448

SEQ ID NO: 450 - RNA, 21 nt, Homo sapiens
agcuacaucuggcuacugggu

SEQ ID NO: 451 - RNA, 22 nt, Homo sapiens
ugccuacugagcugaaacacag

SEQ ID NO: 452 - RNA, 21 nt, Homo sapiens
uucacaguggcuaaguuccgc

SEQ ID NO: 453 - RNA, 99 nt, Homo sapiens
gaaacugggcucaaggugaggggugcuaucugugauugagggacaugguuaauggaauugucucacacagaaaucgcacccgucaccuuggccuacuua

SEQ ID NO: 454 - RNA, 94 nt, Mus musculus
uucacugugggaugagguaguagguuguauaguuuuagggucacacccaccacugggagauaacuauacaaucuacugucuuuccuaaggugau

SEQ ID NO: 455 - RNA, 96 nt, Mus musculus
cugcauguucccagguugagguaguagguuguauaguuuagaguuacaucaagggagauaacuguacagccuccuagcuuuccuugggacuugcac

SEQ ID NO: 456 - RNA, 85 nt, Mus musculus
gcagggugagguaguagguugugugguuucagggcagugauguugccccuccgaagauaacuauacaaccuacugccuucccuga

SEQ ID NO: 457 - RNA, 94 nt, Mus musculus
ugugugcauccggguugagguaguagguuguaugguuuagaguuacacccugggaguuaacuguacaaccuucuagcuuuccuuggagcacacu

SEQ ID NO: 458 - RNA, 95 nt, Mus musculus
acggccuuuggggugagguaguagguuguaugguuuugggcucugccccgcucugcgguaacuauacaaucuacugucuuuccugaaguggccgc

SEQ ID NO: 459 - RNA, 103 nt, Mus musculus
aauggguuccuaggaagagguaguagguugcauaguuuuagggcagagauuuugcccacaaggaguuaacuauacgaccugcugccuuucuuagggccuuauu

SEQ ID NO: 460 - RNA, 93 nt, Mus musculus
cgcgccccccgggcugagguaggagguuguauaguugaggaagacacccgaggagaucacuauacggccuccuagcuuuccccaggcugcgcc

SEQ ID NO: 461 - RNA, 89 nt, Mus musculus
aucagagugagguaguagauuguauaguugugggguagugauuuuacccuguuuaggagauaacuauacaaucuauugccuucccugag

SEQ ID NO: 462 - RNA, 83 nt, Mus musculus; identical to SEQ ID NO: 8

SEQ ID NO: 463 - RNA, 88 nt, Mus musculus
ccaggcugagguaguaguuuguacaguuugagggucuaugauaccacccgguacaggagauaacuguacaggccacugccuugccagg

SEQ ID NO: 464 - RNA, 85 nt, Mus musculus
cuggcugagguaguaguuugugcuguuggucggguugugacauugcccgcuguggagauaacugcgcaagcuacugccuugcuag

SEQ ID NO: 465 - RNA, 77 nt, Mus musculus
gcuugggacacauacuucuuuauaugcccauaugaaccugcuaagcuauggaauguaaagaaguauguauuucaggc

SEQ ID NO: 466 - RNA, 21 nt, Mus musculus
uacauacuucuuuacauucca

SEQ ID NO: 467 - RNA, 80 nt, Mus musculus
ccuguugccacaaacccguagauccgaacuugugcugauucugcacacaagcuugugucuauagguaugugucuguuagg

SEQ ID NO: 468 - RNA, 83 nt, Mus musculus
aggcugcccuggcucaguuaucacagugcugaugcuguccauucuaaagguacaguacugugauaacugaaggauggcagcca

SEQ ID NO: 469 - RNA, 97 nt, Mus musculus
aucugagacugaacugcccuuuuucgguuaucaugguaccgaugcuguagcucugaaagguacaguacugugauagcugaagaauggcggugccauc

SEQ ID NO: 470 - RNA, 86 nt, Mus musculus
uucuuacugcccucggcuucuuuacagugcugccuuguugcauauggaucaagcagcauuguacagggcuaugaaggcauugagac

SEQ ID NO: 471 - RNA, 23 nt, Mus musculus
ccaagugcucagaugcuuguggu

SEQ ID NO: 472 - RNA, 65 nt, Mus musculus
augucaaagugcuaacagugcagguagcuuuuugaguucuacugcagugccagcacuucuuacau

SEQ ID NO: 473 - RNA, 82 nt, Mus musculus
ccugcugggacuaaagugcugacagugcagauagugguccucucugugcuaccgcacuguggguacuugcugcuccagcagg

SEQ ID NO: 474 - RNA, 87 nt, Mus musculus
uucucugugcuuucagcuucuuuacaguguugccuuguggcauggaguucaagcagcauuguacagggcuaucaaagcacagagagc

SEQ ID NO: 475 - RNA, 110 nt, Mus musculus
gaccugucugucuucuguauauacccuguagauccgaauuuguguaaggaauuuuguggucacaaauucguaucuaggggaauauguaguugacauaaacacuccgcuca

SEQ ID NO: 476 - RNA, 68 nt, Mus musculus
uauauacccuguagaaccgaauuugugugguacccacauagucacagauucgauucuaggggaauaua

SEQ ID NO: 477 - RNA, 22 nt, Mus musculus
gagugcuggaauuaaaggcaug

SEQ ID NO: 478 - RNA, 120 nt, Mus musculus
auacucacagucucccagcuggugugagguugggccaggaugaaacccaaggcucuccgaggcuccccaccacacccugcugcugaagacugccuagcaaggcugugccgaguggugugg

SEQ ID NO: 479 - RNA, 21 nt, Mus musculus
cagucuuacuauguagcccua

SEQ ID NO: 480 - RNA, 22 nt, Mus musculus
gaaugaguaacugcuagauccu

SEQ ID NO: 481 - RNA, 23 nt, Mus musculus
ugaguucgaggccagccugcuca

SEQ ID NO: 482 - RNA, 121 nt, Mus musculus
uuuuuuuucuuuuguuuauuuuguuugugacaguugucuguuauguguuccuggcuggcuugguacucauguguagcccaagcuagccucuaacucauggcagucauccugucucagucuc

SEQ ID NO: 483 - RNA, 66 nt, Mus musculus
agcuguggagugugacaaugguguuuguguccaaaccaucaaacgccauuaucacacuaaauagcu

SEQ ID NO: 484 - RNA, 85 nt, Mus musculus; identical to SEQ ID NO: 32

SEQ ID NO: 485 - RNA, 22 nt, Mus musculus
acaggugagguucuugggagcc

SEQ ID NO: 486 - RNA, 24 nt, Mus musculus
ucccugagacccuuuaaccuguga

SEQ ID NO: 487 - RNA, 77 nt, Mus musculus
ugcgcuccccucagucccugagacccuaacuugugauguuuaccguuuaaauccacggguuaggcucuugggagcug

SEQ ID NO: 488 - RNA, 71 nt, Mus musculus
gccuagucccugagacccuaacuugugagguauuuuaguaacaucacaagucagguucuugggaccuaggc

SEQ ID NO: 489 - RNA, 22 nt, Mus musculus
ucccugagacccuaacuuguga

SEQ ID NO: 490 - RNA, 73 nt, Mus musculus
ugacagcacauuauuacuuuugguacgcgcugugacacuucaaacucguaccgugaguaauaaugcgcgguca

SEQ ID NO: 491 - RNA, 22 nt, Mus musculus
ucguaccgugaguaauaaugcg

SEQ ID NO: 492 - RNA, 21 nt, Mus musculus
cauuauuacuuuugguacgcg

SEQ ID NO: 493 - RNA, 70 nt, Mus musculus
ccagccugcugaagcucagagggcucugauucagaaagaucaucggauccgucugagcuuggcuggucgg

SEQ ID NO: 494 - RNA, 22 nt, Mus musculus
ucaggucccuguucaggcgcca

SEQ ID NO: 495 - RNA, 21 nt, Mus musculus
ucacagugaaccggucucuuu

SEQ ID NO: 496 - RNA, 22 nt, Mus musculus
aagcccuuaccccaaaaaguau

SEQ ID NO: 497 - RNA, 79 nt, Mus musculus
cagucuccaccaccuccccugcaaacguccagugaugcagagguaauggacguuggcucugguggugauggacaguccg

SEQ ID NO: 498 - RNA, 64 nt, Mus musculus
gagcucuuuucacauugugcuacugucuaacguguaccgagcagugcaauguuaaaagggcauc

SEQ ID NO: 499 - RNA, 82 nt, Mus musculus
ggcuuguuggacacucuuucccuguugcacuacugugggccucugggaagcagugcaaugaugaaagggcaucugucgggcc

SEQ ID NO: 500 - RNA, 66 nt, Mus musculus
gggcaaccguggcuuucgauuguuacugugggaaccggagguaacagucuacagccauggucgccc

SEQ ID NO: 501 - RNA, 68 nt, Mus musculus
gcuaaagcugguaaaauggaaccaaaucgccucuucaauggauuugguccccuucaaccagcuguagc

SEQ ID NO: 502 - RNA, 119 nt, Mus musculus
ccuccaaagggaguggcccccugcucuggcuggucaaacggaaccaaguccgucuuccugagagguuugguccccuucaaccagcuacagcagggcuggcaaagcucaauauuuggaga

SEQ ID NO: 503 - RNA, 90 nt, Mus musculus
aggccucacuguucucuauggcuuuuuauuccuaugugauucuauugcucgcucauauagggauuggagccguggcguacggugaggaua

SEQ ID NO: 504 - RNA, 100 nt, Mus musculus
agauaaauucacucuagugcuuuauggcuuuuuauuccuaugugaucguaauaaagucucauguagggauggaagccaugaaauacauugugaaaauuca

SEQ ID NO: 505 - RNA, 97 nt, Mus musculus
cgcucugcuguggccuauggcuuuucauuccuaugugauugcugcuccgaacucauguagggcuaaaagccaugggcuacagugaggggcaagcucc

SEQ ID NO: 506 - RNA, 73 nt, Mus musculus
cuucggugacggguauucuuggguggauaauacggauuacguuguuauugcuuaagaauacgcguagucgagg

SEQ ID NO: 507 - RNA, 99 nt, Mus musculus
cucuagcaugguguugugggacagcugguguugugaaucaggccguugccaaucagagaacggcuacuucacaacaccagggccacacugcacugcaag

SEQ ID NO: 508 - RNA, 22 nt, Mus musculus
uggagacgcggcccuguuggag

SEQ ID NO: 509 - RNA, 22 nt, Mus musculus
ucuacagugcacgugucuccag

SEQ ID NO: 510 - RNA, 70 nt, Mus musculus
ccugccagugguuuuacccuaugguagguuacgucaugcuguucuaccacaggguagaaccacggacagg

SEQ ID NO: 511 - RNA, 72 nt, Mus musculus
ggguccaucuuccagugcaguguuggaugguugaaguaugaagcuccuaacacugucugguaaagauggccc

SEQ ID NO: 512 - RNA, 64 nt, Mus musculus
acccauaaaguagaaagcacuacuaacagcacuggaggguguaguguuuccuacuuuauggaug

SEQ ID NO: 513 - RNA, 23 nt, Mus musculus
uguaguguuuccuacuuuaugga

SEQ ID NO: 514 - RNA, 21 nt, Mus musculus
cauaaaguagaaagcacuacu

SEQ ID NO: 515 - RNA, 63 nt, Mus musculus
ccugaggugcagugcugcaucucuggucaguugggagucugagaugaagcacuguagcucagg

SEQ ID NO: 516 - RNA, 70 nt, Mus musculus
cucacgguccaguuuucccaggaaucccuuggaugcuaagauggggauuccuggaaauacuguucuugag

SEQ ID NO: 517 - RNA, 65 nt, Mus musculus
agcucugagaacugaauuccauggguuauaucaaugucagaccugugaaauucaguucuucagcu

SEQ ID NO: 518 - RNA, 109 nt, Mus musculus
gacugagagaacuuuggccaccuggcucugagaacugaauuccauaggcugugagcucuagcagacgcccuagggacucaguucuggugcccggcugugcuauaccauc

SEQ ID NO: 519 - RNA, 99 nt, Mus musculus
agccaguuuggucuuuugagacaaaguucugagacacuccgacucugaguaugauagaagucagugcacuacagaacuuugucucuagaggcugugguc

SEQ ID NO: 520 - RNA, 97 nt, Mus musculus
caggcacccuuagcauuugaggugaaguucuguuauacacucaggcuguggcucugaaagucagugcaucacagaacuuugucucgaaagcuuucua

SEQ ID NO: 521 - RNA, 66 nt, Mus musculus
ggcucuggcuccgugucuucacucccguguuuguccgaggagggagggagggacgggggcggugcu

SEQ ID NO: 522 - RNA, 65 nt, Mus musculus
cccugucucccaacccuuguaccagugcugugccucagacccugguacaggccugggggauaggg

SEQ ID NO: 523 - RNA, 21 nt, Mus musculus
cuagacugaggcuccuugagg

SEQ ID NO: 524 - RNA, 73 nt, Mus musculus
ccgggccuagguucugugauacacuccgacucgggcucuggagcagucagugcaugacagaacuugggcccgg

SEQ ID NO: 525 - RNA, 65 nt, Mus musculus
cuguuaaugcuaauugugauagggguuuuggccucugacugacuccuaccuguuagcauuaacag

SEQ ID NO: 526 - RNA, 84 nt, Mus musculus
cccuuggaguaaaguagcagcacauaaugguuuguggauguugaaaaggugcaggccauacugugcugccucaaaauacaagga

SEQ ID NO: 527 - RNA, 64 nt, Mus musculus
cuguagcagcacaucaugguuuacauacuacagucaagaugcgaaucauuauuugcugcucuag

SEQ ID NO: 528 - RNA, 93 nt, Mus musculus
augucagcggugccuuagcagcacguaaauauuggcguuaagauucugaaauuaccuccaguauugacugugcugcugaaguaagguuggcaa

SEQ ID NO: 529 - RNA, 84 nt, Mus musculus
gucagaauaaugucaaagugcuuacagugcagguagugaugugugcaucuacugcagugagggcacuuguagcauuaugcugac

SEQ ID NO: 530 - RNA, 3849 nt, Mus musculus
ccuagucauacacguggaccuagcagcacccgaagcauugcccaaggauacuugcugagaaggaaguuugccugagugggugaguauauucuaguuuuguagcuaaaacuucuuuugagaccuuugguuuucacuuuugucuuugagcuuguacaacauucuuggucuuuuaagguaugaaauaaauacaguuugaacucuugucauaaaugagugagccacauuuuaacguaguaaaucuacaguggcuuuuggacacuaaccaaauaguuguagacccuucaaguuggagucauagaguauuucuaauuuggggugauacuaagacuuuuuuauguuuuaugacuaauaaacuugaaaaugacuaaacaauaaucauuaaucuugucgaguaucugacaauguggaggacagaagaaagggauugcugccuggucaaugugaggcuuuccucuaaagguaguaccaagucagacugccuuccugauaacaggucucauuuuguaggacccuuauuguguguuuuuuggggaagccuacuguaaaagccaacauuuuaaugggauuguaucuuauauuucuuuaaggaguuuuuuuuuuuuuuuuaaggcuuacauguguccaauuuggaacuucuggcuauuggcuccuccccucccccccuuggguauaagcuguaauugauguuugugacagaauuuagagcuuuggcuuuuuccuuuuugucuaauuauuuauuucaaauuuagcaggaauaaagugaaccucaccuugggacugaagcugugaccagucagaauaaugucaaagugcuuacagugcagguagugaugugugcaucuacugcagugagggcacuuguagcauuaugcugacagcugccucggugggagccacagugggcgcugccucgggcggcacuggcugcguccagucgucggucagucggucgcggggagggccugcuggugcugcgugcuuuuuguucuaaggugcaucuagugcagauagugaaguagacuagcaucuacugcccuaagugcuccuucuggcauaagaaguuauguccucauccaauccaagucaagcaagcauguaggggucucuccauaguuguguuugcagcccucuguuaguuuugcauaguugcacuacaagaagaauguaguugugcaaaucuaugcaaaacugaugguggccugcuauuuacuucaaguguuguuuuuuuuuaaacuaauuuuguauuuuuauugugucgauguagagccugcguggugugugugaugugacagcuucuguagcacuaaagugcuuauagugcagguaguguguagccaucuacugcauuacgagcacuuaaaguacugccagcuguagaacuccagccucgccuggccaucgcccagccaacuguccuguuauugagcacuggucuaugguuaguuuugcagguuugcauccagcuguauaauauucugcugugcaaauccaugcaaaacugacugugguggugaaaagucuguagagaaguaagggaaaaucaaaccccuuucuacacagguugggauuugucgcaaugcuguguuucucuguaugguauugcacuugucccggccuguugaguuugguggggauugugaccagaagaugugaaaaugacaauauugcugaagaugccgauuuccacuguaaaaauguucacgagcugauggaguguaugaacacauucuguaaacugugauuucuauugcuuucguugguguguaaaacgggauuuuuuuaauugaaauuuguguuuuuaaauugugguuuguuuuuacugaaucauuucauuuucguguuugcucgcuggauuuaguaagaaguugugugugggggcuuaggugaaguuaaacugccuuuugaaccugguaugacuucagguuuauuuuacucuaaaacccauuaacaugaguuaagaaaaauguuacuuucuuuggaucucuguuuauuaaagugugacaauucagauggaguguuuucugauaagauuauuccuaauccgugaguucuuaacaguuaacaggugguggccacucuguuaaugugcacacccuguaagaaagaacuauccuuggacuguucuuaccaugugacccccucaagggaaagauggcaaacugauggucaguagagugacagguacacaugacacucgagugcugggucuggagaagcugcaguuaguauuuaggauaacaguauauauaauauauaguucuacauuugggcagcacaguugguuucaggcuaugaauaaaaaucauuggagugaaaccuaaagaaaaguaaaauuaauaaggaagagcugcuaaaaucagguuuaagcugacacuaucuacagagcuaaaguuuuccauaauauguugcuuuuuuuaaaagaaaugcaagaagcugacugauggaucccugagcugccagguaaggauuccacaggccuggguuugauugugagugaccagaauuguacaauuauuacuaaaacagaaacauagucacuucugacuccauucuuaacauuuuuggauuaaaacuuguuucagaugacuaaugaaaacuuccuuuaaaacgugauaaagcucuaaugucagcacgauccacaugauccaugugccuccuauaaagggaggggucugucaccaguguuacggauugaaugcuacguuuaucuuccaacauaggaagccugccagguacuuucuucaauauacuaaauuagaaguuuaaaauuaauaaggucuuaauuaggaccuuuggaugcugagcuugugguaguaauuguguucagaauguuuugaacuuaaauugagauuuaauuuuaguaaggaaugugugucuuagaggccuaguagugaagaggaguuuuccuaggauuuuccucucuccucccuuaauuaagcugcuuucugcacuaagggcuucacgcagcaacgcacuuguucaguuccgcacagggggcuuugcugacugucugcucuaaaacucuguuggcaaaacagcuguggucucucuuagacuuugauuuuguaguaacuagggcauauaguugcuauaagcuuucaggaauggggguggguuacuaaugucuuauguuauaugaaaguaguuaaauuuauccuauauuaagaaggaagcauuauugagacuauauacuguugauuuuacuaaaaacucuaagauguccauuuuaacaggcaaaccuagcacagaaaaccagcuggauuuucccugugcaugguuugaagagucaguccuacauaauugcugacacaaugcaaccugcaacugcccggagaaagaacaguucacuaaaauuuguuguauuuaucauccaauucuguucuguaacugguaacacuaguuugucuggcuuuagagaauaggugaaucucuaaaacaguagaaacagcucaguugggcaagggccguucuaguagcaugccugcuccuuggaguuuuccagauuauuuuguauagccuauuccagauucuugucuaagauugcuuugcucugugcacucagaauuggugugccuuucuauuuguguaaaugauauauaauacauugcuaguugucuaggaucuauucaggaagugcgugcugugaauuuuaaaguaugggaagauuguuaacaaggguuucaauguuuuguuuuuuuuuuucugguaagcuaaaauagaacauugugagguaccugguuuauugugguuacagacuuggaauaauguucauuguuuugugcuaauaaauuguguuaaaauuugguguacauaugagcauuuuuaaauaaaguuauuuuguagcacuga

SEQ ID NO: 531 - RNA, 87 nt, Mus musculus
gguugcuucagugaacauucaacgcugucggugaguuuggaauucaaauaaaaaccaucgaccguugauuguacccuauagcuaacc

SEQ ID NO: 532 - RNA, 80 nt, Mus musculus
aggucacaaucaacauucauugcugucgguggguugaacuguguagaaaagcucacugaacaaugaaugcaacuguggcc

SEQ ID NO: 533 - RNA, 89 nt, Mus musculus
gccaaggguuugggggaacauucaaccugucggugaguuugggcagcucagacaaaccaucgaccguugaguggaccccgaggccugga

SEQ ID NO: 534 - RNA, 75 nt, Mus musculus
accauuuuuggcaaugguagaacucacaccgguaagguaaugggacccggugguucuagacuugccaacuauggu

SEQ ID NO: 535 - RNA, 70 nt, Mus musculus
cuguguauggcacugguagaauucacugugaacagucucagucagugaauuaccgaagggccauaaacag

SEQ ID NO: 536 - RNA, 23 nt, Mus musculus
agaccuacuuaucuaccaacagc

SEQ ID NO: 537 - RNA, 22 nt, Mus musculus
aagguagauagaacaggucuug

SEQ ID NO: 538 - RNA, 69 nt, Mus musculus
ccuuuccuuaucacuuuuccagccagcuuugugacucuaaguguuggacggagaacugauaaggguagg

SEQ ID NO: 539 - RNA, 65 nt, Mus musculus
agggauuggagagaaaggcaguuccugaugguccccucccaggggcuggcuuuccucugguccuu

SEQ ID NO: 540 - RNA, 71 nt, Mus musculus
acuuuccaaagaauucuccuuuugggcuuucucauuuuauuuuaagcccuaaggugaauuuuuugggaagu

SEQ ID NO: 541 - RNA, 21 nt, Mus musculus
caucccuugcaugguggaggg

SEQ ID NO: 542 - RNA, 22 nt, Mus musculus
gcaagggagagggugaagggag

SEQ ID NO: 543 - RNA, 22 nt, Mus musculus
cucucugauggugggugaggag

SEQ ID NO: 544 - RNA, 22 nt, Mus musculus
cuuuggauggagaaagaggggg

SEQ ID NO: 545 - RNA, 22 nt, Mus musculus
aggucaagguucacaggggauc

SEQ ID NO: 546 - RNA, 96 nt, Mus musculus
ugcgugcuuuuuguucuaaggugcaucuagugcagauagugaaguagacuagcaucuacugcccuaagugcuccuucuggcauaagaaguuauguc

SEQ ID NO: 547 - RNA, 22 nt, Mus musculus
ccgcucguacucccgggggucc

SEQ ID NO: 548 - RNA, 22 nt, Mus musculus
agaggugcaguaggcaugacuu

SEQ ID NO: 549 - RNA, 22 nt, Mus musculus
guucugcuccucuggagggagg

SEQ ID NO: 550 - RNA, 22 nt, Mus musculus
caccagucccaccacgcgguag

SEQ ID NO: 551 - RNA, 80 nt, Mus musculus
ggacauuaggagcaaccuccuaggguuguugugagaauuaaaugaacugcagcagccugaggcagggcugggcagagacc

SEQ ID NO: 552 - RNA, 74 nt, Mus musculus
agcgggcaacggaaucccaaaagcagcuguugucuccagagcauuccagcugcacuuggauuucguucccugcu

SEQ ID NO: 553 - RNA, 89 nt, Mus musculus
cgugcacagggcucugaccuaugaauugacagccaguacucuuuucucuccucuggcugccaauuccauaggucacagguauguucacc

SEQ ID NO: 554 - RNA, 66 nt, Mus musculus
gagagcugggucuuugcgggcaagaugagagugucaguucaacuggccuacaaagucccaguccuc

SEQ ID NO: 555 - RNA, 84 nt, Mus musculus
augcccacggucaccuccauaguaccugcagcgugcuugacuuucucuauggugcaguuacuguggcuguggcuguguucgugc

SEQ ID NO: 556 - RNA, 22 nt, Mus musculus
guugcggacagcgcuaggucgg

SEQ ID NO: 557 - RNA, 18 nt, Mus musculus
aucccggacgagccccca

SEQ ID NO: 558 - RNA, 18 nt, Mus musculus
aucccggaagagccccca

SEQ ID NO: 559 - RNA, 22 nt, Mus musculus
cggugggacuuguaguucgguc

SEQ ID NO: 560 - RNA, 20 nt, Mus musculus
ucgauucccugccaaugcac

SEQ ID NO: 561 - RNA, 79 nt, Mus musculus
ggcccagaaucgggguuuugagggcgagaugaguuuguguuuuauccaacuggcccacaaagucccgcuuuugggguca

SEQ ID NO: 562 - RNA, 67 nt, Mus musculus
aucggguguaacagcaacuccauguggacugugcucggauuccaguggagcugcuguuacuucugau

SEQ ID NO: 563 - RNA, 27 nt, Mus musculus
auggaggacugagaagguggagcaguu

SEQ ID NO: 564 - RNA, 73 nt, Mus musculus
ggcugggaagggaggaucugggcaccuggaccagcuccucccugcaggugccagcuccucccuucccagucac

SEQ ID NO: 565 - RNA, 27 nt, Mus musculus
cucugugcugaaugucaaguucugauu

SEQ ID NO: 566 - RNA, 27 nt, Mus musculus
agccgggcagugguggcacacacuuuu

SEQ ID NO: 567 - RNA, 24 nt, Mus musculus
cuauaccaggaugucagcauaguu

SEQ ID NO: 568 - RNA, 94 nt, Mus musculus
acacccaacucuccuggcucuagcagcacagaaauauuggcauggggaagugagucugccaauauuggcugugcugcuccaggcagggugguga

SEQ ID NO: 569 - RNA, 22 nt, Mus musculus
guaguggagacugguguggcua

SEQ ID NO: 570 - RNA, 22 nt, Mus musculus
ugggaaaguucucaggcuucug

SEQ ID NO: 571 - RNA, 21 nt, Mus musculus
acugcagagugagacccuguu

SEQ ID NO: 572 - RNA, 22 nt, Mus musculus
aguccagggcugagucagcgga

SEQ ID NO: 573 - RNA, 22 nt, Mus musculus
uaggaaaguggaagcaguaagu

SEQ ID NO: 574 - RNA, 20 nt, Mus musculus
ggggauguagcucaguggag

SEQ ID NO: 575 - RNA, 22 nt, Mus musculus
ccagugcuguuagaagagggcu

SEQ ID NO: 576 - RNA, 17 nt, Mus musculus
ugagguaguaguuagaa

SEQ ID NO: 577 - RNA, 23 nt, Mus musculus
aagauggagacuuuaacaugggu

SEQ ID NO: 578 - RNA, 102 nt, Mus musculus
ugagccgggacuguugagugaaguagguaguuucauguuguugggccuggcuuucugaacacaacgacaucaaaccaccugauucauggcaguuacugcuuc

SEQ ID NO: 579 - RNA, 75 nt, Mus musculus; identical to SEQ ID NO: 134

SEQ ID NO: 580 - RNA, 23 nt, Mus musculus
ugugucacuggggauaggcuuug

SEQ ID NO: 581 - RNA, 18 nt, Mus musculus
guaaaggcugggcugaga

SEQ ID NO: 582 - RNA, 82 nt, Mus musculus
guaaaggcugggcuuagacguggccuuuggguguggaaugcacuuccguuuguaaccgccaucuaacccuggccuuugacag

SEQ ID NO: 583 - RNA, 23 nt, Mus musculus
ucucacccuauguucucccacag

SEQ ID NO: 584 - RNA, 21 nt, Mus musculus
ucacccuauguucucccacag

SEQ ID NO: 585 - RNA, 22 nt, Mus musculus
acaguagucugcacauugguua

SEQ ID NO: 586 - RNA, 82 nt, Mus musculus
gcagcccucuguuaguuuugcauaguugcacuacaagaagaauguaguugugcaaaucuaugcaaaacugaugguggccugc

SEQ ID NO: 587 - RNA, 87 nt, Mus musculus
cacuggucuaugguuaguuuugcagguuugcauccagcuguauaauauucugcugugcaaauccaugcaaaacugacugugguggug

SEQ ID NO: 588 - RNA, 84 nt, Mus musculus
acuuacgauuaguuuugcagauuugcaguucagcguauaugugaauauauggcugugcaaauccaugcaaaacugauuguggga

SEQ ID NO: 589 - RNA, 90 nt, Mus musculus
cugggccucugugggcaucuuaccggacagugcuggauuucuuggcuugacucuaacacugucugguaacgauguucaaaggugacccac

SEQ ID NO: 590 - RNA, 70 nt, Mus musculus
gccguggccaucuuacugggcagcauuggauagugucugaucucuaauacugccugguaaugaugacggc

SEQ ID NO: 591 - RNA, 69 nt, Mus musculus
cccucgucuuacccagcaguguuugggugcugguugggagucucuaauacugccggguaaugauggagg

SEQ ID NO: 592 - RNA, 76 nt, Mus musculus
gccugguccagugguucuugacaguucaacaguucuguagcacaauugugaaauguuuaggaccacuagacccggc

SEQ ID NO: 593 - RNA, 68 nt, Mus musculus
uggacuucccuuugucauccuaugccugagaauauaugaaggaggcugggaaggcaaagggacguuca

SEQ ID NO: 594 - RNA, 107 nt, Mus musculus
gugugaugugacagcuucuguagcacuaaagugcuuauagugcagguaguguguagccaucuacugcauuacgagcacuuaaaguacugccagcuguagaacuccag

SEQ ID NO: 595 - RNA, 80 nt, Mus musculus
ccuaguagugccaaagugcucauagugcagguaguuuuuauaccacucuacugcagugugagcacuucuaguacuccugg

SEQ ID NO: 596 - RNA, 92 nt, Mus musculus
uguaccaccuugucggauagcuuaucagacugauguugacuguugaaucucauggcaacagcagucgaugggcugucugacauuuugguauc

SEQ ID NO: 597 - RNA, 110 nt, Mus musculus
ccggggcagucccuccaggcucaggacagccacugcccaccgcacacugcguugcuccggacccacugugcgugugacagcggcugaucugucccugggcagcgcgaacc

SEQ ID NO: 598 - RNA, 106 nt, Mus musculus
cugcuuggaccugugaccugugggcuucccuuugucauccuuugccuaggccucugagugaggcaaggacagcaaaggggggcucaguggucaccucuacugcaga

SEQ ID NO: 599 - RNA, 91 nt, Mus musculus
gggcagcgcgccggcaccuuggcucuagacugcuuacugcccgggccgccuucaguaacagucuccagucacggccaccgacgccuggccc

SEQ ID NO: 600 - RNA, 19 nt, Mus musculus
gucuugggaaacggggugc

SEQ ID NO: 601 - RNA, 20 nt, Mus musculus
agaggucuuggggccgaaac

SEQ ID NO: 602 - RNA, 22 nt, Mus musculus
aagggaacgggcuuggcggaau

SEQ ID NO: 603 - RNA, 24 nt, Mus musculus
agcugcgcugcuccugguaacugc

SEQ ID NO: 604 - RNA, 110 nt, Mus musculus; identical to SEQ ID NO: 154

SEQ ID NO: 605 - RNA, 20 nt, Mus musculus
guggagaaggguuccaugug

SEQ ID NO: 606 - RNA, 110 nt, Mus musculus
gugauaauggagcgagauuuucuguugugcuugaucuaaccaugugcuugcgagguaugagaaaaacaugguuccgucaagcaccauggaacgucacgcagcuuucuaca

SEQ ID NO: 607 - RNA, 22 nt, Mus musculus
acgccacauuucccacgccgcg

SEQ ID NO: 608 - RNA, 18 nt, Mus musculus
uugaaccccugaccuccu

SEQ ID NO: 609 - RNA, 22 nt, Mus musculus
caacagcagucgaugggcuguc

SEQ ID NO: 610 - RNA, 95 nt, Mus musculus
accuggcugagccgcaguaguucuucaguggcaagcuuuauguccugacccagcuaaagcugccaguugaagaacuguugcccucugccccuggc

SEQ ID NO: 611 - RNA, 95 nt, Mus musculus
auccaggucuggggcaugaaccuggcauacaauguagauuucuguguuuguuaggcaacagcuacauugucugcuggguuucaggcuaccuggaa

SEQ ID NO: 612 - RNA, 79 nt, Mus musculus
cccucaguggcucaguagccaguguagauccugucuuugguaaucagcagcuacaucuggcuacugggucucugguggc

SEQ ID NO: 613 - RNA, 110 nt, Mus musculus
ucuggccaucugcagugucacgcuccguguauuugacaagcugaguuggacacucugugugguagagugucaguuugucaaauaccccaaguguggcucaugccuaucag

SEQ ID NO: 614 - RNA, 1766 nt, Mus musculus
uggcgcccccgaguggguguggucacguugcccagcaaugggcggugauuggcccugggugguucauucgcagcucgugcgucacgacgccgccagcugaucggagacuggagccggugugugcugggcgcugggaagagacagagcggucggccgugcggacaggucgcagugauuuugcuccucuguccacagcaacccccgcacccagcaucaggaaccugugccucccacacccucaccuggcugagccgcaguaguucuucaguggcaagcuuuauguccugacccagcuaaagcugccaguugaagaacuguugcccucugccccuggcuucguggaggaagaggagaagcagcagcuuugccuaucauccggaagugugucccuccagcacuggguacauggcucugcuguccucauccaacauggagccucagaggugagaaagggcagccuggaagcaacagaggcaggcacaagacaguggaggaccuggccuggaaccacaagggccuauccggacauuggucagagaggcacguagaagccuggagaacaccaggaaagagagcagccagccagccucagugaaagacacgugcuuccagccaucuccucucaggaccugccuuccugggagaugaagggccuccaggaaguauggucccaucucuacccugcaguuucuauaaacagccucaaggagcaugagccaccucugaaaggaaauacacagcaaauucaaaaagagauucaaauguguaacacugugggaaaacauaucuaugacugggguuguagcucaguugguagguuugcuuaacaugcaccaagcccugguucugucuucugcauugcauaaaacugaacagguuggcccaggucugcaaucccggcacucuggaggugguggcaaaggagccuacauucaagguaauccucugcuauacaaugaguucugagccagccugggcuauaugagacugucucaaaaaauaaaacaaaauaaaauaaagcauugguuaguaauucaaagaaagcagauguggcugaaaccguuuucccugaucauaauacaacaagcaaaugaaagccagaagaaggcuccugugccuuguguguggcaguaccaaccauugugagagaugccuuuggaccugguaguuugcugucuuagaaauguauccuaaaauaaggauuugguuauaaaauguucaucucaggguuguaauagagaaaaauggaacgcagcuguuuguuuggaaguccauuccuuuucugcugucaugaaaauguauagcuagggcuugccuaaguaaauuauauucaucugauggugguguucugugcagccauccaaagucuuacagaagaaaaauugaguggaaauauaaauauugaauacuaaaaagauuauaaaaguaugaguuugugacuguuuuuaaaauaugaacacauacuuguaauauauuuuuuuaaaaaccauccaauugaguggaaauauaaauacugaauacuaaaaagauuaugaaaaguaugaguuugugacuguuuuuaaaauaugaaugcauacuuguaauauauuuuuuaaaaaaacacugaaaguggauucaaaauguuaagaaugguuguuuuuguauggugggauaguacaauugugaauuuuccccuuguuuuuucugucuuucuaauuuuuaaauauugugcauugcuuucauauguuaaauaaaauacaaaagacaaauaaauguuuuaaaauuuu

SEQ ID NO: 615 - RNA, 75 nt, Mus musculus
cggacggcugggguuccuggggaugggauuugaugccagucacaaaucacauugccagggauuuccaacugaccc

SEQ ID NO: 616 - RNA, 74 nt, Mus musculus
ggcugcuuggguuccuggcaugcugauuugugacuugagauuaaaaucacauugccagggauuaccacgcaacc

SEQ ID NO: 617 - RNA, 68 nt, Mus musculus
cuccggugccuacugagcugauaucaguucucauuucacacacuggcucaguucagcaggaacaggag

SEQ ID NO: 618 - RNA, 107 nt, Mus musculus
gccucucuccgggcuccgccucccgugccuacugagcugaaacaguugauuccagugcacuggcucaguucagcaggaacaggaguccagcccccuaggagcuggca

SEQ ID NO: 619 - RNA, 84 nt, Mus musculus; identical to SEQ ID NO: 168

SEQ ID NO: 620 - RNA, 90 nt, Mus musculus
aaggccguggccucguucaaguaauccaggauaggcugugcaggucccaaggggccuauucuugguuacuugcacggggacgcgggccug

SEQ ID NO: 621 - RNA, 84 nt, Mus musculus
ggcugcggcuggauucaaguaauccaggauaggcuguguccguccaugaggccuguucuugauuacuuguuucuggaggcagcg

SEQ ID NO: 622 - RNA, 85 nt, Mus musculus
ugcccgggacccaguucaaguaauucaggauagguuguggugcugaccagccuguucuccauuacuuggcucgggggccggugcc

SEQ ID NO: 623 - RNA, 87 nt, Mus musculus
uggccugaggagcagggcuuagcugcuugugagcaagguccacagcaaagucguguucacaguggcuaaguuccgcccccuggaccc

SEQ ID NO: 624 - RNA, 73 nt, Mus musculus
aggugcagagcuuagcugauuggugaacagugauugguuuccgcuuuguucacaguggcuaaguucugcaccu

SEQ ID NO: 625 - RNA, 86 nt, Mus musculus
ggucccuaccuucaaggagcucacagucuauugaguugccuuucugauucucccacuagauugugagcugcuggagggcaggcacu

SEQ ID NO: 626 - RNA, 80 nt, Mus musculus
uucaaucugugguacucaaacugugugacauuuuguucuuuguaagaagugccgcagaguuuguaguguugccgauugag

SEQ ID NO: 627 - RNA, 84 nt, Mus musculus
uuccauauagccauacucaaaauggaggcccuaucuaagcuuuuaaguggaaagugcuucccuuuuguguguugccauguggag

SEQ ID NO: 628 - RNA, 22 nt, Mus musculus
gaggguuggguggaggcucucc

SEQ ID NO: 629 - RNA, 21 nt, Mus musculus
agggcccccccucaauccugu

SEQ ID NO: 630 - RNA, 76 nt, Mus musculus
auauguauguauguauguaugugugcaugugcaugugcauguaugcauauugcauguauauauuaugcauacaugu

SEQ ID NO: 631 - RNA, 88 nt, Mus musculus
accccuuagaggaugacugauuucuuuugguguucagagucaauagaauuuucuagcaccaucugaaaucgguuauaaugauugggga

SEQ ID NO: 632 - RNA, 71 nt, Mus musculus
aggaagcugguuucauauggugguuuagauuuaaauagugauugucuagcaccauuugaaaucaguguucu

SEQ ID NO: 633 - RNA, 88 nt, Mus musculus; identical to SEQ ID NO: 182

SEQ ID NO: 634 - RNA, 86 nt, Mus musculus
ccugcuaacggcugcucugacuuuauugcacuacuguacuuuacagcgagcagugcaauaguauugucaaagcauccgcgagcagg

SEQ ID NO: 635 - RNA, 97 nt, Mus musculus
uuuccugcuggcugcgggugcucugacuagguugcacuacugugcugugagaagcagugcaaugguauugucaaagcaucugggaccagccucgaag

SEQ ID NO: 636 - RNA, 69 nt, Mus musculus
ccaccacuuaaacgugguuguacuugcuuuagaccuaagaaaguaagugcuuccauguuuuggugaugg

SEQ ID NO: 637 - RNA, 66 nt, Mus musculus
ccuuuacuuuaacauggaggcacuugcugugcauuuaaaaauaagugcuuccauguuugagugugg

SEQ ID NO: 638 - RNA, 64 nt, Mus musculus
ggacagagugugugugucuguguggagacaggaguugcccacacauggcacucaacucugcagg

SEQ ID NO: 639 - RNA, 71 nt, Mus musculus
gcgacuguaaacauccucgacuggaagcugugaagccacaaaugggcuuucagucggauguuugcagcugc

SEQ ID NO: 640 - RNA, 96 nt, Mus musculus
cuaagccaaguuucaguucauguaaacauccuacacucagcugucauacaugcguuggcugggauguggauguuuacgucagcugucuuggaguau

SEQ ID NO: 641 - RNA, 89 nt, Mus musculus
accauguuguaguguguguaaacauccuacacucucagcugugagcucaagguggcugggagaggguuguuuacuccuucugccaugga

SEQ ID NO: 642 - RNA, 84 nt, Mus musculus
gagugacagauauuguaaacauccuacacucucagcugugaaaaguaagaaagcugggagaaggcuguuuacucucucugccuu

SEQ ID NO: 643 - RNA, 82 nt, Mus musculus
aagucugugucuguaaacauccccgacuggaagcuguaagccacagccaagcuuucagucagauguuugcugcuacuggcuc

SEQ ID NO: 644 - RNA, 92 nt, Mus musculus
gggcagucuuugcuacuguaaacauccuugacuggaagcuguaagguguugagaggagcuuucagucggauguuuacagcggcaggcugcca

SEQ ID NO: 645 - RNA, 106 nt, Mus musculus
ugcuccuguaacucggaacuggagaggaggcaagaugcuggcauagcuguugaacugagaaccugcuaugccaacauauugccaucuuuccugucugacagcagcu

SEQ ID NO: 646 - RNA, 63 nt, Mus musculus
cuagggggcaggagccggagcccucugcugaacugacagacgcucugcuuugcucccccagau

SEQ ID NO: 647 - RNA, 82 nt, Mus musculus
gccucgccgcccuccgccuucucuucccgguucuucccggagucgggaaaagcuggguugagagggcgaaaaaggauguggg

SEQ ID NO: 648 - RNA, 95 nt, Mus musculus
ccucguugacuccgaagggcugcagcagcaauucauguuuuggaguauugccaagguucaaaacaugaagcgcugcaacaccccuucguggggaa

SEQ ID NO: 649 - RNA, 20 nt, Mus musculus
ccacugccccaggugcugcu

SEQ ID NO: 650 - RNA, 23 nt, Mus musculus
cgcauccccuagggcauuggugu

SEQ ID NO: 651 - RNA, 95 nt, Mus musculus
cucaucugucuguugggcugggggcagggccuuugugaaggcggguuaugcucagaucgccucugggcccuuccuccagucccgaggcagauuua

SEQ ID NO: 652 - RNA, 97 nt, Mus musculus
cugucucggagccuggggcaggggggcaggaggggcucagggagaaaguaucuacagccccuggcccucucugcccuuccguccccuguccccaagu

SEQ ID NO: 653 - RNA, 21 nt, Mus musculus
gccccugggccuauccuagaa

SEQ ID NO: 654 - RNA, 22 nt, Mus musculus
cuagguauggucccagggaucc

SEQ ID NO: 655 - RNA, 23 nt, Mus musculus
ucaagagcaauaacgaaaaaugu

SEQ ID NO: 656 - RNA, 21 nt, Mus musculus
gaacggcgucaugcaggaguu

SEQ ID NO: 657 - RNA, 23 nt, Mus musculus
ugagcgccucggcgacagagccg

SEQ ID NO: 658 - RNA, 23 nt, Mus musculus
ucccuguccuccaggagcucacg

SEQ ID NO: 659 - RNA, 22 nt, Mus musculus
uccgucucaguuacuuuauagc

SEQ ID NO: 660 - RNA, 22 nt, Mus musculus
uuauaaagcaaugagacugauu

SEQ ID NO: 661 - RNA, 23 nt, Mus musculus
ucucacacagaaaucgcacccgu

SEQ ID NO: 662 - RNA, 22 nt, Mus musculus
aggggugcuaucugugauugag

SEQ ID NO: 663 - RNA, 96 nt, Mus musculus
acccaaguccaggccugcugaccccuaguccagugcuugugguggcuacugggcccugaacuaggggucuggagaccuggguuugaucuccacagg

SEQ ID NO: 664 - RNA, 22 nt, Mus musculus
gcugaccccuaguccagugcuu

SEQ ID NO: 665 - RNA, 22 nt, Mus musculus
cccugggaggagacguggauuc

SEQ ID NO: 666 - RNA, 102 nt, Mus musculus
ccagcugugaguaauucuuuggcagugucuuagcugguuguugugaguauuagcuaaggaagcaaucagcaaguauacugcccuagaagugcugcacauugu

SEQ ID NO: 667 - RNA, 22 nt, Mus musculus
aaucacuaacuccacugccauc

SEQ ID NO: 668 - RNA, 23 nt, Mus musculus
aggcaguguaauuagcugauugu

SEQ ID NO: 669 - RNA, 77 nt, Mus musculus
agucuaguuacuaggcaguguaguuagcugauugcuaauaguaccaaucacuaaccacacagccagguaaaaagacu

SEQ ID NO: 670 - RNA, 99 nt, Mus musculus
agaugccuugcuccuacaagaguaaagugcaugcgcuuugggacagugaggaaaauaauguucacaaagcccauacacuuucacccuuuaggagaguug

SEQ ID NO: 671 - RNA, 99 nt, Mus musculus
cauggcaccuccguuucccugaggagcccuuugagccuggagugaaaaaaaaaaacaggucaagaggcgccugggaacuggagaagaguguuaaacuuc

SEQ ID NO: 672 - RNA, 70 nt, Mus musculus
gaagcuuaucagaaucuccagggguacuuaguauuugaaaagucccccaggugugauucugauuuguuuc

SEQ ID NO: 673 - RNA, 22 nt, Mus musculus
aacacaccuguucaaggauuca

SEQ ID NO: 674 - RNA, 24 nt, Mus musculus
aauccuuggaaccuaggugugaau

SEQ ID NO: 675 - RNA, 75 nt, Mus musculus
uguuaucagguggaacacgaugcaauuuugguugguguaauaggaggaaaauugcacgguauccaucuguaaacc

SEQ ID NO: 676 - RNA, 87 nt, Mus musculus
accgcagggaaaaugagggacuuuugggggcagauguguuuccauuccgcuaucauaaugccccuaaaaauccuuauugcucuugca

SEQ ID NO: 677 - RNA, 22 nt, Mus musculus
auauaauacaaccugcuaagug

SEQ ID NO: 678 - RNA, 64 nt, Mus musculus
ccccgcgacgagccccucgcacaaaccggaccugagcguuuuguucguucggcucgcgugaggc

SEQ ID NO: 679 - RNA, 82 nt, Mus musculus
ugguauuuaaaagguggauauuccuucuaugguuacgugcuuccuggauaaucauagaggaacauccacuuuuucaguauca

SEQ ID NO: 680 - RNA, 86 nt, Mus musculus
uuugguauuuaaaagguggauauuccuucuauguuuaugcuuuuugugauuaaacauagaggaaauuucacguuuucagugucaaa

SEQ ID NO: 681 - RNA, 75 nt, Mus musculus; identical to SEQ ID NO: 243

SEQ ID NO: 682 - RNA, 22 nt, Mus musculus
gaauguugcucggugaaccccu

SEQ ID NO: 683 - RNA, 81 nt, Mus musculus
ggguacuugaggagagguugucugugaugaguucgcuuuauuaaugacgaauauaacacagauggccuguuuucaauacca

SEQ ID NO: 684 - RNA, 82 nt, Mus musculus
ugguacuuggagagauaguagaccguauagcguacgcuuuaucugugacguauguaacacgguccacuaacccucaguauca

SEQ ID NO: 685 - RNA, 23 nt, Mus musculus
ugaggggcagagagcgagacuuu

SEQ ID NO: 686 - RNA, 85 nt, Mus musculus
aaagugcuuuggaaugacacgaucacucccguugagugggcacccaagaagccaucgggaaugucguguccgcccagugcucuuu

SEQ ID NO: 687 - RNA, 22 nt, Mus musculus
uuugaaccaucacucgacuccu

SEQ ID NO: 688 - RNA, 20 nt, Mus musculus
aggcaguguuguuagcuggc

SEQ ID NO: 689 - RNA, 22 nt, Mus musculus
auugggaacauuuugcaugcau

SEQ ID NO: 690 - RNA, 22 nt, Mus musculus
aaaccguuaccauuacugaguu

SEQ ID NO: 691 - RNA, 75 nt, Mus musculus
cuuuaccuaauuuguuguccaucauguaaaacauaaaugaugauagacaccauauaagguagaggaagguucacu

SEQ ID NO: 692 - RNA, 23 nt, Mus musculus
uauuuagaauggcacugauguga

SEQ ID NO: 693 - RNA, 22 nt, Mus musculus
uauuuagaauggcgcugaucug

SEQ ID NO: 694 - RNA, 81 nt, Mus musculus
uauauguguugauguguguguacauguacaugugugaauaugauauacaaauacauacacgcacacauaagacacauauga

SEQ ID NO: 695 - RNA, 22 nt, Mus musculus
gauguguguguacauguacaua

SEQ ID NO: 696 - RNA, 21 nt, Mus musculus
auacagacacaugcacacaca

SEQ ID NO: 697 - RNA, 23 nt, Mus musculus
ugugugcaugugcauguguguaa

SEQ ID NO: 698 - RNA, 23 nt, Mus musculus
uguguguguacauguacauguga

SEQ ID NO: 699 - RNA, 73 nt, Mus musculus
cugugugcguaagugccugcauguauaugcguguauauuuuaugcauauacauacacacaccuacacacacau

SEQ ID NO: 700 - RNA, 73 nt, Mus musculus
ccgugugcguaagugccugcauguauaugcguguauauuuuaugcauauacauacacacaccaacacacacau

SEQ ID NO: 701 - RNA, 97 nt, Mus musculus
ccuuugugcauaagugcgugcauguauauguguguauauuuuaugcauauacauacacacaccuauacacacaugcacacagacaugcgagaauggc

SEQ ID NO: 702 - RNA, 85 nt, Mus musculus
ccugugugcauaagugcgcgcauguauaugcguguauauuuuaugcauauacauacacacaccuacacacacaugcacacagaca

SEQ ID NO: 703 - RNA, 21 nt, Mus musculus
auauacacacacacaccuaca

SEQ ID NO: 704 - RNA, 22 nt, Mus musculus
auaagugugugcauguauaugu

SEQ ID NO: 705 - RNA, 75 nt, Mus musculus
cagugcucuucuuggacuggcacuggugaguuaaacuaaauacaaccaguaccuuucugagaagaguaaagcuca

SEQ ID NO: 706 - RNA, 22 nt, Mus musculus
ucaggcucaguccccucccgau

SEQ ID NO: 707 - RNA, 22 nt, Mus musculus
agucauacacggcucuccucuc

SEQ ID NO: 708 - RNA, 128 nt, Mus musculus
cagccagcucugaucucgcccucccugagggguccuguacugagcugccccgagguccuucacugugcucagcucggggcagcucaguacaggaugcgucagggugggagacaacggggaacaagcca

SEQ ID NO: 709 - RNA, 82 nt, Mus musculus
ugguacuuggagagugguuaucccuguccucuucgcuucacucaugccgaaucguacagggucauccacuuuuucaguauca

SEQ ID NO: 710 - RNA, 107 nt, Mus musculus
acugcugcaguggcagcuugguugucauaugugugaugacacuuucuaaagucuuccagaaugacaccacauauauggcagcuaaacuguuacauggaacaacaagu

SEQ ID NO: 711 - RNA, 84 nt, Mus musculus
uggaaaguucauuguucgacaccauggaucuccaggugggucacguuuagauauacaccaaccuggaggacuccaugcuguuga

SEQ ID NO: 712 - RNA, 85 nt, Mus musculus
uugauacuugaaggagagguuguccguguugucuucucuuuauuuaugaugaaacauacacgggaaaccucuuuuuuaguaucaa

SEQ ID NO: 713 - RNA, 63 nt, Mus musculus
aaagaaguugcccauguuauuuuucgcuuuuauuugugacgaaacaaacauggugcacuucuu

SEQ ID NO: 714 - RNA, 79 nt, Mus musculus
aguguucgaauggagguugcccaugguguguucauuuuauuuaugaugaguauuacauggccaaucuccuuucggcacu

SEQ ID NO: 715 - RNA, 84 nt, Mus musculus
ccugcccccgccccagcagcacacugugguuuguacggcacuguggccacguccaaaccacacugugguguuagagcgagggua

SEQ ID NO: 716 - RNA, 92 nt, Mus musculus
cuccucugcucccccucucuaauccuugcuaucugggugcuuagugcuaucucaaugcaaugcaccugggcaaggguucagagaaggugagc

SEQ ID NO: 717 - RNA, 22 nt, Mus musculus
aaugcacccgggcaaggauuug

SEQ ID NO: 718 - RNA, 71 nt, Mus musculus
ugcccuagcagcgggaacaguacugcagugaguguuuggugcccuggaguauuguuuccacugccugggua

SEQ ID NO: 719 - RNA, 21 nt, Mus musculus
ugauugacauuucuguaaugg

SEQ ID NO: 720 - RNA, 23 nt, Mus musculus
cggcgccccacggagccccgagc

SEQ ID NO: 721 - RNA, 96 nt, Mus musculus
cagauuugcuuuuucucuuccaugccuugaguguaggaccguugacaucuuaauuacccucccacacccaaggcuugcaggagagcaagccuucuc

SEQ ID NO: 722 - RNA, 22 nt, Mus musculus
ccucccacacccaaggcuugca

SEQ ID NO: 723 - RNA, 22 nt, Mus musculus
caugccuugaguguaggaccgu

SEQ ID NO: 724 - RNA, 74 nt, Mus musculus
uacuugaggagaaauuauccuugguguguuggcucuuuuggaugaaucauacaaggauaauuucuuuuugagua

SEQ ID NO: 725 - RNA, 22 nt, Mus musculus
ugugacagauugauaacugaaa

SEQ ID NO: 726 - RNA, 16 nt, Mus musculus
augguggcacggaguc

SEQ ID NO: 727 - RNA, 78 nt, Mus musculus
ugcgggcgugugagugugugugugugagugugugucgcuccaaguccacgcucaugcacacacccacacgcccgcacg

SEQ ID NO: 728 - RNA, 22 nt, Mus musculus
cacgcucaugcacacacccaca

SEQ ID NO: 729 - RNA, 22 nt, Mus musculus
uccgagccugggucucccucuu

SEQ ID NO: 730 - RNA, 22 nt, Mus musculus
ggggguccccggugcucggauc

SEQ ID NO: 731 - RNA, 98 nt, Mus musculus
aggaacagcuauguacugcacaacccuaggagggggugccauucacauagaguauaauugaauggcgccacuaggguugugcaguguacagccuacac

SEQ ID NO: 732 - RNA, 22 nt, Mus musculus
ugguaagcugcagaacaugugu

SEQ ID NO: 733 - RNA, 94 nt, Mus musculus
agaacagggucuccuugaggggccucugccucuauccaggauuauguuuuuaugaccaggaggcugaggucccuuacaggcggccucuuacucu

SEQ ID NO: 734 - RNA, 22 nt, Mus musculus
ggcugcagcgugaucgccugcu

SEQ ID NO: 735 - RNA, 92 nt, Mus musculus
guggguacuggccucggugcugguggagcagugagcacgccauacauuauaucugugacaccugccacccagcccaaggccccuaggcccac

SEQ ID NO: 736 - RNA, 66 nt, Mus musculus
gguaagugugccucgggugagcaugcacuuaauguagguguaugucacucggcucggcccacuacc

SEQ ID NO: 737 - RNA, 121 nt, Mus musculus
cagcccgggauuuguguguugcuugcucuauauguguguauacuugugugugcauguauauguguguauaugaauauacauauacauacacacccauauacacacgcaugcaugcacacac

SEQ ID NO: 738 - RNA, 98 nt, Mus musculus
guuugugcaugugcguauaguugugugugcauguauauguguguauaugaauauacauauacauacacacccauauauacacgcauucauaugaacac

SEQ ID NO: 739 - RNA, 20 nt, Mus musculus
auuuguguguggaugugugu

SEQ ID NO: 740 - RNA, 100 nt, Mus musculus
gguuuggaggugggccugacaucccugaguguauguggugaaccugaacuugcccuggguuuccucauauccauucaggagugucagcugccucuucgcu

SEQ ID NO: 741 - RNA, 21 nt, Mus musculus
uccgguucucagggcuccacc

SEQ ID NO: 742 - RNA, 100 nt, Mus musculus; identical to SEQ ID NO: 411

SEQ ID NO: 743 - RNA, 91 nt, Mus musculus
uggagccugaggggcucacagcucugguccuuggagcuccagagaaaauguugcuccggggcugaguucugugcaccccccuugcccucca

SEQ ID NO: 744 - RNA, 23 nt, Mus musculus
uccggggcugaguucugugcacc

SEQ ID NO: 745 - RNA, 100 nt, Mus musculus
ggccuagucaucacccugagccuugcacugagaugggagugguguaaggcucagguaugcacagcucccaucucagaacaaggcucgggugugcucagcu

SEQ ID NO: 746 - RNA, 22 nt, Mus musculus
cuguaugcccuaaccgcucagu

SEQ ID NO: 747 - RNA, 21 nt, Mus musculus
gggcaucugcugacauggggg

SEQ ID NO: 748 - RNA, 21 nt, Mus musculus
cugcagucacagugaagucug

SEQ ID NO: 749 - RNA, 19 nt, Mus musculus
aguuuucccuucaagucaa

SEQ ID NO: 750 - RNA, 21 nt, Mus musculus
ucaauggcugaggugaggcac

SEQ ID NO: 751 - RNA, 22 nt, Mus musculus
cuauccuggaaugcagcaauga

SEQ ID NO: 752 - RNA, 22 nt, Mus musculus
aaaggcuaggcucacaaccaaa

SEQ ID NO: 753 - RNA, 21 nt, Mus musculus
aucucuuugagcgccucacuc

SEQ ID NO: 754 - RNA, 19 nt, Mus musculus
cugaaaauguugccugaag

SEQ ID NO: 755 - RNA, 18 nt, Mus musculus
gcgugugcuugcuguggg

SEQ ID NO: 756 - RNA, 109 nt, Mus musculus
ucaucucugcccucacuguaagggagggugguggcuaugugggugggacagggauguucaguugcuaaagacauucucguuuccuucccucaguguccccagaugguga

SEQ ID NO: 757 - RNA, 79 nt, Mus musculus
uucacugggaguaaggcuccuuccugugcuugcaggggagaaauacgaacugcacgcgggaaccgaguccacccccagu

SEQ ID NO: 758 - RNA, 109 nt, Mus musculus
cgggacaaggugaguggggugguuggcauggguugcccauggggacucgacgcugugcccacagccuccugauguccuccucacgcaugcccacccuuuaccccgcucc

SEQ ID NO: 759 - RNA, 21 nt, Mus musculus
agacaugugcucugcuccuag

SEQ ID NO: 760 - RNA, 22 nt, Mus musculus
agagaaacccugucucaaaaaa

SEQ ID NO: 761 - RNA, 109 nt, Mus musculus
cuguguuugaaauggggacugcccucaaggagcuuacaaucuagcuggggguagaugacuugcacuugaacacaacuagacugugagcuucuagagggcaggggccuua

SEQ ID NO: 762 - RNA, 22 nt, Mus musculus
ccaagucuuggggagaguugag

SEQ ID NO: 763 - RNA, 109 nt, Mus musculus
ucuccgcuucuccuucacccgggcgguacccgcuccggcgccggcccgcgggacgccgcggcguccgugcgccgaugcgagucacccccggguguugcgaguucgggga

SEQ ID NO: 764 - RNA, 22 nt, Mus musculus
aucucggcuacagaaaaauguu

SEQ ID NO: 765 - RNA, 18 nt, Mus musculus
aucucgcuggggccucca

SEQ ID NO: 766 - RNA, 21 nt, Mus musculus
cagugcaauuaaaagggggaa

SEQ ID NO: 767 - RNA, 71 nt, Mus musculus
uugaucuacguagauugguaccuaucauguaaaucauguaagcaugagagaugccauucuauguagauuaa

SEQ ID NO: 768 - RNA, 62 nt, Mus musculus
cuguauucagauuggugccugucauguuuauaagaaugaaagacaccaagcugaguagagua

SEQ ID NO: 769 - RNA, 100 nt, Mus musculus
ggcugggcaaggugcggggcuagggcuaacagcagucuuacugacgguuuccuggaaaccacacacaugcuguugccacuaaccucaaccuuacucgguc

SEQ ID NO: 770 - RNA, 22 nt, Mus musculus
gcagcagggugaaacugacaca

SEQ ID NO: 771 - RNA, 22 nt, Mus musculus
ccagcugggaagaaccaguggc

SEQ ID NO: 772 - RNA, 94 nt, Mus musculus
gccaccuucugugcccccagcaccacgugucugggccacgugagcaacgccacgugggccugacguggagcuggggccgcaggggucugauggc

SEQ ID NO: 773 - RNA, 108 nt, Mus musculus
uuggauguuggccuaguucuguguggaagacuagugauuuuguuguuuuuagauaacuaaaacgacaacaaaucacagucugccauauggcacaggccaccucuacag

SEQ ID NO: 774 - RNA, 111 nt, Mus musculus
aggagcggaguacgugagccagugcuauguggaagacuugugauuuuguuguucugauaugauaugacaacaagucacagccagccucauagcguggacuccuaucaccuu

SEQ ID NO: 775 - RNA, 22 nt, Mus musculus
ugugaguuguuccucaccugga

SEQ ID NO: 776 - RNA, 20 nt, Mus musculus
gaauugaucaggacauaggg

SEQ ID NO: 777 - RNA, 81 nt, Mus musculus
aacuuguuagaagguuacuuguuaguucaggaccucauuacuuucugccugaacuauugcaguagccuccuaacugguuau

SEQ ID NO: 778 - RNA, 76 nt, Mus musculus
uuagcccugcggccccacgcaccaggguaagagagacucacuuccugcccuggcccgagggaccgacuggcugggc

SEQ ID NO: 779 - RNA, 21 nt, Mus musculus
ccugaaaauacugaggcuaug

SEQ ID NO: 780 - RNA, 85 nt, Mus musculus
guagaggagauggcgcaggggacacaagguaggccuugcgggucuguggacccuuggacauguguccucuucucccuccucccag

SEQ ID NO: 781 - RNA, 22 nt, Mus musculus
gcaugacaccacacuggguaga

SEQ ID NO: 782 - RNA, 78 nt, Mus musculus
ugcacugcaauacucagauugauaugagucacuuccuauugcauguuacuccauccucucugaguagaguaaggcaca

SEQ ID NO: 783 - RNA, 78 nt, Mus musculus
ugcaguacaauauucagagagauaacagucacaucuuuucuaaaguaacugugucuuuucugaauagaguaauguuca

SEQ ID NO: 784 - RNA, 22 nt, Mus musculus
uaacugcaacagcucucaguau

SEQ ID NO: 785 - RNA, 89 nt, Mus musculus; identical to SEQ ID NO: 432

SEQ ID NO: 786 - RNA, 80 nt, Mus musculus
cuuucuacacagguugggauuugucgcaaugcuguguuucucuguaugguauugcacuugucccggccuguugaguuugg

SEQ ID NO: 787 - RNA, 22 nt, Mus musculus
agguggggauugguggcauuac

SEQ ID NO: 788 - RNA, 88 nt, Mus musculus
agucaugggggcuccaaagugcuguucgugcagguaguguaauuaccugaccuacugcugagcuagcacuucccgagcccccaggaca

SEQ ID NO: 789 - RNA, 108 nt, Mus musculus
cugcacaugcuggggugagguaguaaguuguauuguugugggguagggauuuuaggccccaguaagaagauaacuauacaacuuacuacuuuccuugguguguggcau

SEQ ID NO: 790 - RNA, 65 nt, Mus musculus
cauaaacccguagauccgaucuuguggugaaguggaccgcgcaagcucguuucuaugggucugug

SEQ ID NO: 791 - RNA, 70 nt, Mus musculus; identical to SEQ ID NO: 445

SEQ ID NO: 792 - RNA, 87 nt, Rattus norvegicus
ugccuacucagagcacauacuucuuuauguacccauaugaacauagaaugcuauggaauguaaagaaguguguauuuuggguaggua

SEQ ID NO: 793 - RNA, 87 nt, Rattus norvegicus
ugcgcuccccucagucccugagacccuaacuugugauguuuaccguuuaaauccacggguuaggcucuugggagcugcgagucgugc

SEQ ID NO: 794 - RNA, 88 nt, Rattus norvegicus
uggccaccuggcccugagaacugaauuccauaggcugugaacucuagcagaugcccuagggacucaguucuggugccuggcugugcua

SEQ ID NO: 795 - RNA, 22 nt, Rattus norvegicus
gaaguucuguuauacacucagg

SEQ ID NO: 796 - RNA, 78 nt, Rattus norvegicus
ugcuucugugugauauguuugauauuagguuguuaaauuaugaaccaacuaaaugucaaacauucucacagcagugag

SEQ ID NO: 797 - RNA, 110 nt, Rattus norvegicus
ggcuacagcccuucuucaugugacucguggacuucccuuugucauccuaugccugagaauauaugaaggaggcugggaaggcaaagggacguucaauugucaucacuggc

SEQ ID NO: 798 - RNA, 23 nt, Rattus norvegicus
cuucuccuggcucuccucccuuu

SEQ ID NO: 799 - RNA, 72 nt, Rattus norvegicus
guagugccaaagugcucauagugcagguagguuuugcugcacucuacugcagugugagcacuucugguacuc

SEQ ID NO: 800 - RNA, 22 nt, Rattus norvegicus
agaguugcgucuggacgucccg

SEQ ID NO: 801 - RNA, 82 nt, Rattus norvegicus
gggcuuucaagucacuagugguuccguuuaguagaugguuuuugcauuguuucaaaauggugcccuagugacuacaaagccc

SEQ ID NO: 802 - RNA, 22 nt, Rattus norvegicus
agggcuuagcugcuugugagca

SEQ ID NO: 803 - RNA, 81 nt, Rattus norvegicus
cuucaggaagcugguuucauauggugguuuagauuuaaauagugauugucuagcaccauuugaaaucaguguucuuggugg

SEQ ID NO: 804 - RNA, 88 nt, Rattus norvegicus; identical to SEQ ID NO: 182

SEQ ID NO: 805 - RNA, 19 nt, Rattus norvegicus
ccuugaggggcaugagggu

SEQ ID NO: 806 - RNA, 96 nt, Rattus norvegicus
augugaccgugccucucacccuuccauaucuagucucugagaaaaaugaagacuggauuccaugaagggaugugaggccuggaaacuggagcuuua

SEQ ID NO: 807 - RNA, 22 nt, Rattus norvegicus
cccugaacuaggggucuggaga

SEQ ID NO: 808 - RNA, 19 nt, Rattus norvegicus
ugucccucugggucgccca

SEQ ID NO: 809 - RNA, 92 nt, Rattus norvegicus
gagccccugcuggugggcgcggggcggggguuucagguggucucgcgguggccgcccgacugucccucugggucgcccagcuggggaguucc

SEQ ID NO: 810 - RNA, 24 nt, Rattus norvegicus
uucacaaagcccauacacuuucac

SEQ ID NO: 811 - RNA, 81 nt, Rattus norvegicus
cauggcaccuccauuucccugaggagcccuuugagccugaggugaaaaaaaaacaggucaagaggcgccugggaacuggag

SEQ ID NO: 812 - RNA, 21 nt, Rattus norvegicus
agaguaguagguugcauagua

SEQ ID NO: 813 - RNA, 61 nt, Rattus norvegicus
agcgagguugcccuuuguauauucgguuuauugacaugggauauacaagggcaagcucucu

SEQ ID NO: 814 - RNA, 20 nt, Rattus norvegicus
aauguugcucggugaacccc

SEQ ID NO: 815 - RNA, 86 nt, Rattus norvegicus
cauguguauauauguguguguguauguccauguguguauaugaauauacauacacacacacauacacacacgugcaagcacacaca

SEQ ID NO: 816 - RNA, 106 nt, Rattus norvegicus
acugcuacaguggcagcuugguugucguaugcgugaugacacguucucguguauuccagaaugacaucacauauauggcagcuaaacuguuacaggaacaacaagu

SEQ ID NO: 817 - RNA, 22 nt, Rattus norvegicus
caugccuugaguguaggacugu

SEQ ID NO: 818 - RNA, 19 nt, Rattus norvegicus
gugucuguuuccugccgga

SEQ ID NO: 819 - RNA, 59 nt, Rattus norvegicus
cuggcuggggaaaaagauuggauagaaaacauuauucuauucauuuacuccccagccua

SEQ ID NO: 820 - RNA, 97 nt, Rattus norvegicus
uguuggccuaguucuguguggaagacuagugauuuuguuguuuuuagauaacuaagacgacaacaaaucacagucugccauauggcacaggccaccu

SEQ ID NO: 821 - RNA, 76 nt, Rattus norvegicus
ugcagugcuguauucagauuggugccugucauguuuauaagaaugaaagacgccaaacuggguagaguggagcuca

SEQ ID NO: 822 - RNA, 78 nt, Rattus norvegicus
ggugcgugaggugguugaccagagagcacacgcuauauuugugccguuugugaccugguccacuaacccucaguaucu

SEQ ID NO: 823 - RNA, 22 nt, Rattus norvegicus
gcaugacaccauacuggguaga

SEQ ID NO: 824 - RNA, 77 nt, Rattus norvegicus
ugcaguacaauauucagagugguagcagucacuuuauucuaaaguaacuguggcauuucugaauagaguaauguuca

SEQ ID NO: 825 - RNA, 81 nt, Rattus norvegicus
cccauuggcauaaacccguagauccgaucuuguggugaaguggaccgcacaagcucguuucuaugggucuguggcagugug

SEQ ID NO: 826 - RNA, 60 nt, Homo sapiens
ccuggaugaugauagcaaaugcugacugaacaugaaggucuuaauuagcucuaacugacu

SEQ ID NO: 827 - RNA, 57 nt, Homo sapiens
gaugaccccagguaacucugagugugucgcugaugccaucaccgcagcgcucugacc

SEQ ID NO: 828 - RNA, 57 nt, Mus musculus
cuaaaauagcuggaauuaccggcagauugguaguggugagccuaugguuuucugaag

SEQ ID NO: 829 - RNA, 48 nt, Mus musculus
gcuguacugacuugaugaaaguacuuuugaacccuuuuccaucugaug

SEQ ID NO: 830 - RNA, 255 nt, Homo sapiens
gugcucgcuucggcagcacauauacuaaaauuggaacgauacagagaagauuagcauggccccugcgcaaggaugacacgcaaauucgugaagcguuccauauuuuuacugcccuccaugcccugccccacaaacgcucugauaacagucugucccugucucucuccugcugcuccuauggaagcgaaguuuuccgcuccugcagaaagcaaaguuacgacucagagacggcugaggaugacaucagcgaugugc

SEQ ID NO: 831 - RNA, 76 nt, Homo sapiens
acaaugaugacuuaaauuacuuuuugccguuuacccagcugagguugucuuugaagaaauaauuuuaagacugaga

SEQ ID NO: 832 - RNA, 111 nt, Mus musculus
aaagacuagucaagugcaguagugagaaggggggaaaguguagaacaggaguucaaucuguaacugacugugaacaaucaauugagauaacucacuaccuucggaccagcc

(19) United States
(12) Patent Application Publication
(10) Pub. No.: US 2016/0076098 A1
(43) Pub. Date: Mar. 17, 2016

(54) METHODS OF DIAGNOSING AND TREATING CHRONIC PAIN

(71) Applicants: PHILADEPPHIA HEALTH & EDUCATION CORPORATION D/B/A DREXEL UNIVERSITY OF MEDICINE, Philadelphia, PA (US); DREXEL UNIVERSITY, Philadelphia, PA (US)

(72) Inventors: Seena AJIT, West Windsor, NJ (US); Ahmet SACAN, Drexel Hill, PA (US); Guillermo ALEXANDER, Bryn Mawr, PA (US); Robert J. SCHWARTZMAN, Marco Island, FL (US); Marguerite MCDONALD, Newark, DE (US)

(73) Assignee: Drexel University, Philadelphia, PA (US)

(21) Appl. No.: 14/783,766

(22) PCT Filed: Apr. 11, 2014
(86) PCT No.: PCT/US14/33830

§ 371 Date: Oct. 9, 2015

Related U.S. Application Data
(60) Provisional application No. 61/811,256, filed on Apr. 12, 2013.
(60) Provisional application No. 61/811,374, filed on Apr. 12, 2013.
(60) Provisional application No. 61/886,142, filed on Oct. 3, 2013.

(51) Int. Cl. C12Q 1/68; A61K 31/135

(52) U.S. Cl. CPC C12Q 1/6883; A61K 31/135; C12Q 2600/178; C12Q 2600/106

(57) ABSTRACT

The invention includes compositions and methods useful for the diagnosis, prognosis, treatment, assessment, and characterization of inflammation or pain (e.g., neuropathic pain) in a subject in need thereof, based upon the expression level of at least one miRNA that is associated with inflammation or pain. In one aspect, the invention relates to compositions and methods for the prediction of a subject's responsiveness of a treatment of inflammation or pain.

### CROSS REFERENCE TO RELATED APPLICATIONS

This application claims priority to and the benefit of U.S. Provisional Application Nos. 61/811,256, filed Apr. 12, 2013; 61/811,374, filed Apr. 12, 2013; and 61/886,142, filed Oct. 3, 2013, which are incorporated herein by reference in their entireties.

### STATEMENT REGARDING FEDERALLY SPONSORED RESEARCH OR DEVELOPMENT

This invention was made with government support under grant numbers R21NS082991-01 awarded by the National Institutes of Health. The Government therefore has certain rights in this invention.

### BACKGROUND OF THE INVENTION

Complex regional pain syndrome (CRPS) is a chronic neuropathic pain syndrome that predominantly occurs after an injury, such as fracture. The pain experienced by CRPS patients can be severely debilitating. Generally, CRPS affects one or more extremities, and can affect the skin, muscles, joints, and bones. Chronic pain and inflammation can spread systemically beyond the initial injury. Clinical symptoms include sensory (e.g., pain and hyperalgesia) autonomic (e.g., alterations in skin temperature, color, increased sweating) and motor (e.g., tremor, dystonia) disturbances. Patients with CRPS have measurable increases in circulating cytokines and dysregulated expression of multiple miRNAs. Treatments, particularly in moderate-severe cases, are often ineffective and provide little relief. Ketamine, a widely used anesthetic, is one of the treatment options being pursued for CRPS. However, not all CRPS patients respond to ketamine therapy, which is expensive, involved, and potentially dangerous.

There is a need in the art for compositions and methods for the detection, treatment, and prediction of treatment outcome for Complex regional pain syndrome (CRPS) and symptoms thereof (e.g., neuropathic pain). The present invention addresses these unmet needs.

### SUMMARY OF THE INVENTION

As described below, the present invention features compositions and methods for diagnosing and treating pain (e.g., neuropathic pain) and/or inflammation. The compositions and methods of the invention are particularly useful for treating Complex regional pain syndrome (CRPS) and symptoms thereof.

In one aspect, the invention provides a method of diagnosing neuropathic pain in a subject, the method involving: determining the level of at least one microRNA in a biological sample of the subject, where the at least one microRNA comprises at least one microRNA selected from the group consisting of hsa-miR-31, hsa-miR-636, and hsa-miR-16-1#; and comparing the level of the at least one microRNA in the biological sample with the level of the at least one miRNA or in a comparator, where when the level of the at least one microRNA in the biological sample is different than the level of the at least one miRNA in the comparator, the subject is diagnosed with neuropathic pain.

In one aspect, the invention provides a method of determining the severity of neuropathic pain in a subject, the method involving: determining the level of at least one microRNA in a biological sample of the subject, where the at least one microRNA comprises at least one microRNA selected from the group consisting of hsa-miR-31, hsa-miR-636, and hsa-miR-16-1#; and comparing the level of the at least one microRNA in the biological sample with the level of the at least one miRNA in a comparator, where the greater the difference between the level of the at least one microRNA in the biological sample and the level of the at least one miRNA in the comparator, the greater the severity of neuropathic pain.

In one aspect, the invention provides a method of evaluating the progression of neuropathic pain in a subject, the method involving: determining the level of at least one microRNA in a biological sample of the subject at a first time point, where the at least one microRNA comprises at least one microRNA selected from the group consisting of hsa-miR-31, hsa-miR-636, and hsa-miR-16-1#; comparing the level of the at least one microRNA in the biological sample at the first time point with the level of the at least one microRNA in a comparator; determining the level of at least one microRNA in the biological sample at a second time point; comparing the level of the at least one microRNA in the biological sample at the second time point with the level of the at least one microRNA in a comparator; where when the difference in the level of the at least one microRNA in the biological sample at the second time point, as compared with the comparator, is greater than the difference in the level of the at least one microRNA in the biological sample at the first time point, as compared with the comparator, the neuropathic pain is progressing.

In one aspect, the invention provides a method of evaluating a treatment of neuropathic pain in a subject in need thereof, the method involving: determining the level of at least one microRNA in a biological sample of the subject at a first time point, where the at least one microRNA comprises at least one microRNA selected from the group consisting of hsa-miR-31, hsa-miR-636, hsa-miR-16-1#, hsa-miR-337-3p, hsa-miR-605, hsa-miR-597, RNU44.A, hsa-miR-650; comparing the level of the at least one microRNA in the biological sample at the first time point with the level of the at least one miRNA in a comparator; determining the level of at least one microRNA in the biological sample at a second time point; and comparing the level of the at least one microRNA in the biological sample at the second time point with the level of the at least one miRNA in a comparator, where when the difference in the level of the at least one microRNA in the biological sample at the first time point, as compared with the comparator, is greater than the difference in the level of the at least one microRNA in the biological sample at the second time point, as compared with the comparator, the treatment of neuropathic pain is reducing neuropathic pain.

In one aspect, the invention provides a method of predicting the responsiveness of a treatment of neuropathic pain in a subject, the method involving: determining the level of at least one microRNA in a biological sample of the subject; and comparing the level of the at least one microRNA in the biological sample with the level of the at least one miRNA or in a comparator, where when the level of the at least one microRNA in the biological sample is different than the level of the at least one miRNA in the comparator, the subject is predicted to respond to treatment of neuropathic pain.

In one aspect, the invention provides a method of treating neuropathic pain involving administering to a subject in need thereof an effective amount of a therapeutic agent that modulates the expression and/or activity of at least one miRNA selected from the group consisting of hsa-miR-337-3p, hsa-miR-605, hsa-miR-597, RNU44.A, and hsa-miR-650.

In one aspect, the invention provides a method of diagnosing inflammation or pain in a subject, the method involving: determining the level of at least one exosomal miRNA in a biological sample of the subject, where the at least one exosomal miRNA comprises at least one exosomal miRNA selected from the group consisting of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201; and comparing the level of the at least one exosomal miRNA in the biological sample with the level of the at least one miRNA in a comparator, where when the level of the at least one exosomal miRNA in the biological sample is different than the level of the at least one miRNA in the comparator, the subject is diagnosed with inflammation or pain.

In one aspect, the invention provides a method of determining the severity of inflammation or pain in a subject, the method involving: determining the level of at least one exosomal miRNA in a biological sample of the subject, where the at least one exosomal miRNA comprises at least one exosomal miRNA selected from the group consisting of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201; and comparing the level of the at least one exosomal miRNA in the biological sample with the level of the at least one miRNA in a comparator, where the greater the difference between the level of the at least one exosomal miRNA in the biological sample and the level of the at least one miRNA in the comparator, the greater the severity of inflammation or pain.

In one aspect, the invention provides a method of evaluating a treatment of inflammation or pain in a subject in need thereof, the method involving: determining the level of at least one exosomal miRNA in a biological sample of the subject, where the at least one exosomal miRNA comprises at least one exosomal miRNA selected from the group consisting of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201; comparing the level of the at least one exosomal miRNA in the biological sample at the first time point with the level of the at least one miRNA in a comparator; administering a treatment; determining the level of at least one exosomal miRNA in the biological sample at a second time point after treatment is administered; and comparing the level of the at least one exosomal miRNA in the biological sample at the second time point with the level of the at least one miRNA in a comparator; where when the difference in the level of the at least one exosomal miRNA in the biological sample at the first time point, as compared with the comparator, is greater than the difference in the level of the at least one exosomal miRNA in the biological sample at the second time point, as compared with the comparator, the treatment of inflammation or pain is reducing inflammation or pain.

In one aspect, the invention provides a method of treating inflammation or pain involving administering to a subject in need thereof an effective amount of a therapeutic agent that modulates the level, expression or activity of at least one exosomal miRNA selected from the group consisting of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

In various embodiments of any of the aspects described herein, the pain or neuropathic pain is complex regional pain syndrome (CRPS) or a symptom thereof. In various embodiments of any of the aspects described herein, the subject is human (e.g., a human subject having CRPS). In various embodiments of any of the aspects delineated herein, the pain is neuropathic pain and/or pain associated with complex regional pain syndrome (CRPS).

In various embodiments of any of the aspects described herein, determining the level of the at least one microRNA and/or exosomal miRNA utilizes at least one or more technique involving reverse transcription, PCR and/or a microarray. In various embodiments of any of the aspects described herein, the comparator is at least one comparator selected from the group consisting of a positive control, a negative control, a normal control, a wild-type control, a historical control, and a historical norm. In various embodiments of any of the aspects described herein, the level of the at least one miRNA and/or exosomal miRNA is higher than the level of the at least one miRNA and/or exosomal miRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%. In various embodiments, the level of the at least one miRNA and/or exosomal miRNA is lower than the level of the at least one miRNA and/or exosomal miRNA in the comparator by at least 10%, by at least 20%, by at least 30%, by at least 40%, by at least 50%, by at least 60%, by at least 70%, by at least 80%, by at least 90%, or by at least 100%.

In various embodiments of any of the aspects described herein, the method involves obtaining a biological sample from the subject. In various embodiments of any of the aspects described herein, the method further involves stratifying the subject for inclusion in a clinical trial based upon the severity of the subject's inflammation, pain (e.g., neuropathic pain), and/or symptom associated with CRPS. In various embodiments of any of the aspects described herein, the method further involves modifying the subject's treatment for inflammation or pain (e.g., neuropathic pain). In various embodiments of any of the aspects described herein, the method further involves treating the subject for inflammation or pain (e.g., neuropathic pain). In various embodiments, the method further involves continuing to treat the subject for inflammation, pain, and/or neuropathic pain. In various embodiments of any of the aspects delineated herein, the therapeutic agent is one or more of a small molecule, antibody, antibody fragment, peptide, peptidomimetic, nucleic acid, antisense molecule, miRNA, or ribozyme. In various embodiments, the therapeutic agent inhibits the expression and/or activity of the at least one miRNA. In other various embodiments, the therapeutic agent enhances the expression and/or activity of the at least one miRNA.

In various embodiments of any of the aspects described herein, the treatment involves administering an NMDA receptor antagonist. In various embodiments, the subject has CRPS. In various embodiments, the subject is undergoing, has undergone or will undergo treatment involving administration of an NMDA receptor antagonist. In particular embodiments, the NMDA receptor antagonist is one or more of ketamine, memantine, dizocilpine, phencyclidine, APV (AP5), amantadine, dextromethorphan, dextrorphan, AP7, riluzole, tiletamine, midafotel, aptiganel, methoxetamine, MK-801, ifenprodil, conantokin, and NVP-AAM077. In certain embodiments, the comparator is a control known to not to respond to the treatment. In various embodiments, the method further involves treating the subject for neuropathic pain and/or pain associated with CRPS.

In various embodiments of any of the aspects delineated herein, the miRNA includes one or more of hsa-miR-31, hsa-miR-636, and hsa-miR-16-1#. In various embodiments of any of the aspects delineated herein, the miRNA includes one or more of hsa-miR-31, hsa-miR-636, hsa-miR-16-1#, hsa-miR-337-3p, hsa-miR-605, hsa-miR-597, RNU44.A, and hsa-miR-650. In various embodiments of any of the aspects delineated herein, the miRNA includes one or more of hsa-miR-337-3p, hsa-miR-605, hsa-miR-597, RNU44.A, and hsa-miR-650.

In various embodiments of any of the aspects delineated herein, the exosomal miRNA includes one or more of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

Compositions and articles defined by the invention were isolated or otherwise manufactured in connection with the examples provided below. Other features and advantages of the invention will be apparent from the detailed description, and from the claims.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a Circos diagram demonstrating the correlation of pain score and three miRNAs before treatment. Three miRNAs with strongest correlation (represented by thickness of the band) are shown. Negative correlations are shown in blue and positive correlation is represented in red.

FIG. 2 is a clustergram of the samples and the significantly differentially expressed miRNAs in responders and non-responders before treatment. Red, high; black, average; green, low. (nb=non-responders before treatment; rb=responders before treatment).

FIG. 3 is a clustergram of the samples and the table of significant differentially expressed miRNAs in good responders before and after treatment. Red, high; black, average; green, low. (ra=responders after treatment; rb=responders before treatment).

FIG. 4 is a graph depicting the ketamine treatment regimen given to CRPS patients.

FIG. 5 is a table depicting the pain scores and change in pain of treated patients.

FIG. 6 is a model depicting cross talk between analgesic and endocrinal systems.

FIG. 7 is a graph showing that non-responders to ketamine therapy have increased expression of POMC mRNA in blood relative to the responders and healthy controls. Shown is relative expression POMC mRNA in blood samples from CRPS patients (responders and non-responders to ketamine therapy) before treatment and healthy controls and before and after receiving ketamine therapy. The expression of POMC mRNA was normalized to 18 s. Responders; n=7, non-responders; n=4 and control; n=4. Results represent mean±SEM. The statistical significance was calculated using one-way ANOVA. *: p<0.05 relative to responders and control and relative to responders.

FIG. 8 is a graph showing no significant difference in the basal (pre-treatment) plasma levels of β-endorphin between patients and healthy controls. Ketamine therapy induced an increase in β-endorphin in responders but not the non-responders. Shown are β-endorphin levels in the plasma from CRPS patients (responders and non-responders to ketamine therapy) before treatment and healthy controls and in CRPS patients before and after ketamine therapy. Responders; n=11, non-responders; n=6 and control; n=5. Results represent mean±SEM. The statistical significance was calculated using one-way ANOVA*: p<0.05 relative to non-responders after treatment.

FIG. 9 is a graph showing no difference in the levels of ACTH between patients and control, responders and non-responders to ketamine therapy. Shown are ACTH levels in plasma from CRPS patients (responders and non-responders to ketamine therapy) before treatment and healthy controls and in CRPS patients before and after receiving ketamine therapy. Responders; n=10, non-responders; n=5 and control; n=6. Results represent mean±SEM

FIG. 10, comprising FIGS. 10A through 10C depicts correlations between the analgesic response, relative expression of POMC mRNA and β-endorphin level in response to ketamine. FIG. 10A is a graph depicting the stratification of POMC mRNA and plasma levels of β-endorphin based on response to ketamine therapy. FIG. 10C is a graph depicting the relationship between the relative expression of the POMC mRNA and the analgesic response. FIG. 10C is a 3D representation of the relationship between the analgesic response, POMC mRNA and β-endorphin.

FIG. 11, comprising FIGS. 11A and 11B, depicts correlations between analgesic response, changes in relative expression of POMC (Δ-POMC) and the plasma levels of β-endorphin (Δ β-endorphin) in response to ketamine. FIG. 11A is a table depicting correlations between analgesic response, Δ-POMC and Δ β-endorphin in response to ketamine for the responders. FIG. 11B is a table depicting correlations between analgesic response, Δ-POMC and Δ β-endorphin in response to ketamine for the non-responders. Negative correlation between the analgesic response and Δ-POMC was observed in responders only. The statistical significance was calculated Pearson Correlation coefficient. *: p<0.05.

FIG. 12 is a graph showing that non-responders to ketamine therapy have a lower Body Mass Index (BMI). The BMI of responders and non-responders to ketamine treatment are shown. Results represent mean±SEM *: p<0.05.

FIG. 13 is a schematic summarizing the differences in POMC pathway between responders and non-responders before (left panel) and after (right panel) ketamine therapy.

FIG. 14 is a schematic showing corticotropin-releasing factor receptor (CRHR) targeting by mir-34a.

FIG. 15 is a graph showing that mir-34a binds CRHR in a reporter assay.

FIG. 16 depicts a model showing a potential modulatory role for miR-548d-5p in ketamine metabolism.

FIG. 17 is a graph showing that miR-548d-5p can bind to the 3′ UTR of UDP-GT but not CYP3A4 in a reporter assay.

FIG. 18 depicts a model showing the role of differential miRNA expression in CRPS patients resistant to ketamine therapy. Without being bound to a particular theory, a decrease in miR-548d-5p is involved in resistance to ketamine therapy through pharmacokinetic modulation. Without being bound to a particular theory, a reduction in miR-34a contributes to ketamine resistance and alterations in POMC-beta-endorphin pathway. Dysregulation of POMC derived peptides link BMI to treatment response.

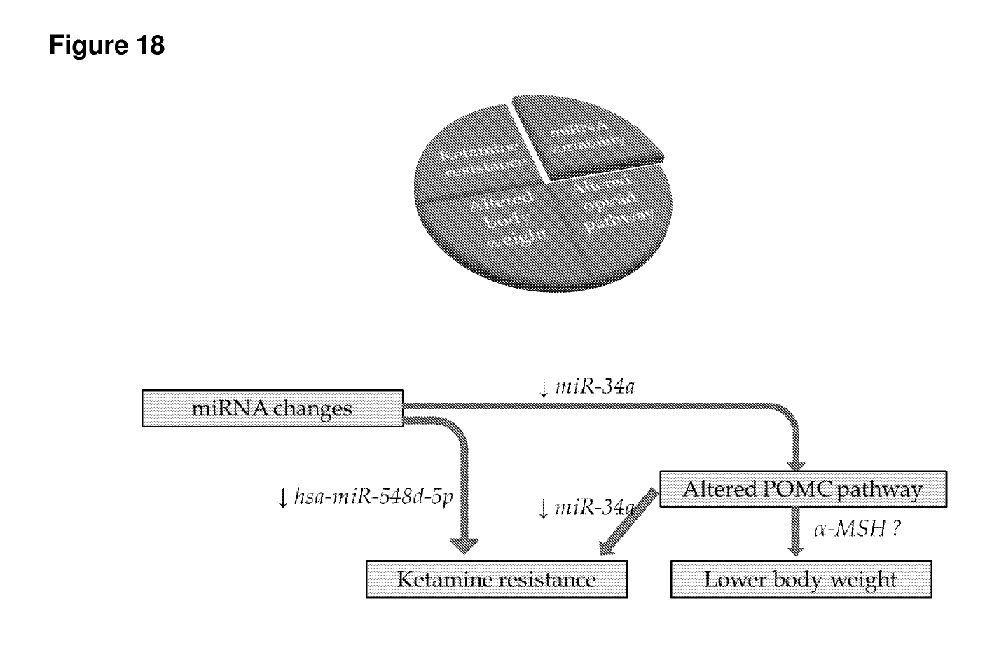

FIG. 19 is a table showing predicted miRNA targets for miRNAs differentially expressed in responders and non-responders.

FIG. 20 is a graph showing that miR-605 binds CXCR5 3′ UTR in a reporter assay.

FIG. 21 is a graph showing that CXCR5 transcripts were elevated in non-responders and that non-responders did not have elevated CXCR5 transcripts.

FIG. 22 is a graph showing that IL13Rα1 transcripts were elevated in non-responders.

FIG. 23 is a graph showing no significant alteration in the level of CXCL13 in the plasma from CRPRS patients.

FIG. 24, comprising FIGS. 24A to 24C, are clustergrams showing differentially expressed miRNAs comparing control and non-responders. FIG. 24A is a clustergram of the control samples and the significant differentially expressed miRNAs in non-responders before treatment. FIG. 24B is a clustergram of the control samples and the significant differentially expressed miRNAs in non-responders after treatment. FIG. 24C is a clustergram of the control samples and the significant differentially expressed miRNAs in non-responders combined.

FIG. 25 is a Circos diagram showing the correlation of selected parameters and miRNAs from ketamine study.

FIG. 26 is a model linking miRNA signature to treatment response.

FIG. 27, comprising FIGS. 1A-1C, provides the introduction and objectives of the studies described herein. Exosomal microRNAs (miRNAs) are small noncoding RNAs that bind mRNA targets via a complementary seed sequence and repress translation. miRNAs circulate in bodily fluids such as blood and can be used as biomarkers in various diseases. A previous study analyzing miRNA levels and inflammatory markers in the blood of patients with CRPS showed an increase in inflammatory markers and differential expression of 18 miRNAs circulating in the blood (Orlova et al., 2011, J Transl Med 9:195). The objectives of the studies described herein included characterizing alterations in miRNA, mRNA, and cytokines in exosomes secreted by RAW 264.7 cells in response to inflammatory stimulus, determine the effect of inflammatory stimuli on exosome-mediated intercellular communication in vitro and in vivo, and determining if miRNA alterations seen in CRPS patients are reflected in the exosomal fraction of blood. FIG. 1A depicts some of the signs of CRPS, which include sensory (pain and hyperalgesia), autonomic (alterations in skin temperature, color, increased sweating) and motor (tremor, dystonia) disturbances. FIG. 1B depicts the results of experiments showing that the inflammatory markers VEGF, IL1Ra, and MCP1 were significantly increased in CRPS patients vs. control samples, with p values 0.0002, 0.0004, and 0.0005, respectively. The levels of IL-4, IL-5, IL-6, 11-8, and TNFα also showed an increase that did not reach statistical significance in this study. FIG. 1C is a schematic representation of exosome formation. Exosomes arise from cytosolic multivesicular bodies (MVBs) which fuse with the plasma membrane to release exosomes (Ludwig and Giebel, 2011, The International J Biochemistry & Cell Biol 44:11-5).

FIG. 28, comprising FIGS. 28A to 28E, depicts characterization of exosome morphology and specificity. FIG. 28A shows transmission electron microscopy images of exosomes purified from RAW 264.7 cells before (left) and after (right) LPS treatment. TEM after staining with uranyl acetate indicates RAW 264.7 cell-derived exosomes are intact after purification (scale=500 nm). Exosomes and fell within the size range of 30-100 nm (scale 500 nm). FIG. 28B depicts TEM images showing that exosomes purified from RAW 264.7 cells before (left) and after (right) LPS treatment are specific for CD81 (scale 100 nm). Exosomes were incubated with anti-CD81 and gold-labeled anti-Rabbit IgG. FIG. 28C shows that 2 commonly detected exosomal proteins, HSP70 (mwpred=70 kDa) and TSG101 (mwpred=43 kDa, mwobs=48 kDa), are present in exosomes and absent in exosome-free cell culture media (media−lane). Exosomal lysate and media was obtained from stimulated (exosome+lane) or unstimulated (exosome−lane) RAW 264.7 cells. FIG. 28D is a bioanalyzer trace and gel showing integrity of total exosomal RNA from LPS-stimulated RAW 264.7 cells run on the eukaryotic total RNA pico chip. FIG. 28E is a bioanalyzer trace of small exosomal RNAs from LPS-stimulated RAW 264.7 cells. The traces followed a typical profile of exosomal RNA.

FIG. 29, comprising FIGS. 29A to 29C, show that exosomal miRNAs are differentially expressed in LPS-stimulated macrophages. FIG. 29A is a heat map showing LPS-responsive exosomal miRNAs (10 of 433 detected miRNAs in RAW 264.7 cell-derived exosomes with significant alterations after LPS stimulation are shown). Samples are labeled minus for control (exosomes from unstimulated RAW 264.7 cells) and plus for exosomes derived from LPS-treated RAW 264.7 cells (n=4) (log 10fc red=high, black=average, green=low). Significance was determined by applying a P value cutoff of 0.05 to the results of a paired-samples t test. FIG. 29B is a heat map showing LPS-responsive exosomal miRNAs (THP1 cell-derived exosomes with significant alterations after LPS stimulation are shown).

FIG. 29B is a table showing LPS-responsive exosomal miRNAs (miRNAs in THP1 cell-derived exosomes with significant alterations after LPS stimulation are shown).

FIG. 30, comprising, FIGS. 4A to 4D depicts statistical analysis of exosomal RNA sequencing data from naive and LPS-stimulated macrophages. FIG. 4A is a graph showing density analysis of RNA sequencing data and that the RNA population profiles differed after LPS treatment (FPKM, fragments per kilobase of exon per million). FIG. 4B is a volcano plot showing significantly different genes detected in exosomes after LPS stimulation compared with naive. Red points indicate significantly differentially expressed transcripts (p≦0.05). FIG. 4C is a Venn diagram of genes that were common and differentially expressed after LPS treatment. The numbers outside the circles indicate the total numbers of genes and noncoding RNAs that were detected. FIG. 4D depicts the percentage of detected transcripts by RNA type, showing that the majority of transcripts detected are protein coding. lincRNA, long intergenic noncoding RNA; lncRNA, long noncoding RNA; miRNA, microRNA; snoRNA, small nucleolar RNA.

FIG. 31 is a gene ontology pie chart showing the distribution of exosomal mRNAs categorized by cellular function based on global reactome pathways. Of the 15,883 genes, 5,445 are represented here.

FIG. 32 depicts qPCR validation of mRNAs detected by NGS. The mRNA levels of 4 cytokines and 2 transcription factors from exosomes secreted by naive and LPS-stimulated RAW 264.7 cells were normalized to Gapdh (n=3) **p<0.01.

FIG. 33, comprising FIGS. 9A and 9B, depicts the results of experiments evaluating miRNA target binding validation. FIG. 9A shows graphs depicting relative luciferase expression of four putative miR-939 targets: TNFAIP1, NOS2A, TNFα, and VEGFA. FIG. 9B shows graphs depicting relative luciferase expression of three miR-532 targets: CXCL3, PTGER2, and PTGER3.

FIG. 34 depicts the results of experiments showing that exosomal cytokines increased after LPS stimulation. FIG. 34A depicts quantification of exosomal protein content before and after LPS stimulation, using an array of 40 cytokines. FIG. 34B depicts that ten cytokines were upregulated in exosomes after LPS treatment. FIG. 34C is a graph depicting quantification of the cytokines upregulated in exosomes after LPS treatment. The relative intensity of detectable cytokines in exosomes secreted from LPS-stimulated cells (orange) or untreated cells (green) was measured after lysis in 1×RIPA buffer and incubation with a Mouse Cytokine Array Panel (R & D Systems) spotted with antibodies against 40 cytokines. The graph shows the mean intensity normalized to the reference spots (n=3). Data are presented as +/−S.E.M.

FIG. 35 Exosomes purified from LPS-stimulated cells caused activation of NF-κB in naive cells. Secreted exosomes were purified from RAW 264.7 cell media after 24 hr incubation±1 μg/ml LPS. Exosomes were incubated at 4 concentrations (based on protein content) with RAW-blue reporter cells and QUANTI-Blue assay was performed at 24 hr. Columns 1-8 are exosomes from (+) LPS-stimulated cells or (−) untreated cells and columns 9-10 are media control for NF-KB activation. The average of 3 experiments was analyzed by ANOVA and a Bonferroni posttest to determine P<0.001.

FIG. 36, comprising FIGS. 36A to 36C, depicts that exosomes from LPS-stimulated macrophages reduced paw thickness in a CFA model of inflammatory pain. FIG. 11A is a schematic of the design of the experiment. Paw thickness and pain behavior measurements before and after each injection. After baseline paw thickness was measured, the CFA model was established in 8-week-old male C57BL/6 mice by intraplantar injection (arrow) into the hind paw. Paw thickness was measured and then a second injection (arrow) of PBS or 20 μl of exosomes (0.5 g) derived from LPS-stimulated or naive RAW 264.7 cells were administered by intraplantar injection to the right hind paw 3 hr after the CFA injection (n=9). FIG. 36B is a graph showing that one injection of exosomes from LPS-stimulated macrophages (black circle) in CFA-treated paw decreased paw thickness at 24 hr compared with exosomes from naive RAW 264.7 cells (gray circles) or PBS injection (triangle). Statistical analysis performed was one-way ANOVA and Bonferroni posttest.

FIG. 36C is a graph showing that animals treated with saline did not show any increase in paw thickness with a second injection of macrophage-derived exosomes. Data shown are mean±SEM.

FIG. 37, comprising FIGS. 37A and 37B, depicts that macrophage-derived exosomes reduced thermal hyperalgesia induced by CFA. FIG. 37A show graphs depicting that macrophage-derived exosomes did not have an effect on mechanical allodynia. Mechanical allodynia was measured by von Frey filaments before and after the CFA model was established in 8-week-old male C57BL/6 mice. A second injection of PBS (white) or exosomes from naive (gray) or LPS-stimulated (black) RAW 264.7 cells was given 3 hr after CFA treatment. Paw withdrawal thresholds were measured at 1, 2, and 5 days and then weekly for 21 days following exosome injection (n=9). Macrophage-derived exosomes had no effect on mechanical allodynia in the CFA-treated animals (right panel) and did not cause hypersensitivity in saline-treated animals as measured by von Frey filaments (right panels).

FIG. 37B show graphs depicting that macrophage-derived exosomes reduced thermal hyperalgesia. Thermal hyperalgesia was measured with Hargreaves direct heat source in CFA-treated (left panel) or saline-treated (right panel) animals. Although exosomes obtained from LPS-stimulated macrophages decreased the paw withdrawal latency immediately upon administration, the effect was transient. At 24 hr, exosomes from LPS-stimulated cells reduced thermal hypersensitivity induced by CFA; by 48 hr exosomes derived from untreated and LPS-stimulated RAW 264.7 cells reduced thermal hypersensitivity. This protective effect lasted for up to 10 days. Data shown are mean±SEM. Statistical analysis was determined by ANOVA with Bonferroni posttest. *P<0.05. **P.<0.01. ***P<0.001.

FIG. 38 depicts miRNA profiling of CRPS exosomes. A heat map is provided showing 127 of 503 detected miRNAs in human serum exosomes with significant alterations in patients with CRPS. Samples are labeled “C” for control subject and “P” for patient with CRPS, (log 10fc red=high, black=average, green=low) (n=6). Significance was determined by applying the Benjamini-Hochberg false discovery rate correction to the results of a 2-tailed t test.

### DETAILED DESCRIPTION

The present invention relates to the discovery that the expression and/or levels of some microRNAs (miRNAs) and/or miRNAs in exosomes are associated with inflammation and pain, including neuropathic pain, such as in complex regional pain syndrome (CRPS).

In one embodiment, the present invention relates to the discovery that the expression levels of some miRNAs are associated with responsiveness to treatment of neuropathic pain.

In one embodiment, the present invention relates to the discovery that the expression levels of some miRNAs are associated with successful treatment of neuropathic pain.

Thus, in various embodiments described herein, the methods of the invention relate to methods of diagnosing a subject as having pain (e.g., neuropathic pain) and/or inflammation, methods of assessing a subject's risk of having or developing pain (e.g., neuropathic pain) and/or inflammation, methods of assessing the severity of a subject's pain (e.g., neuropathic pain) and/or inflammation, methods of predicting a subject's response to treatment of pain (e.g., neuropathic pain) and/or inflammation, methods of evaluating the efficacy of a treatment of pain (e.g., neuropathic pain) and/or inflammation in a subject, and methods of stratifying a subject having pain (e.g., neuropathic pain) and/or inflammation for assignment in a clinical trial.

In some embodiments, the miRNAs associated with pain and/or inflammation are up-regulated, while in other embodiments, the miRNAs associated with pain and/or inflammation are down-regulated.

In some embodiments, the miRNAs associated with responsiveness to treatment are up-regulated, while in other embodiments, the miRNAs associated with responsiveness to treatment are down-regulated.

In some embodiments, the miRNAs associated with successful treatment are up-regulated, while in other embodiments, the miRNAs associated with successful treatment are down-regulated.

Thus, the invention relates to compositions and methods useful for the detection and quantification of miRNAs, including miRNAs in exosomes, for the diagnosis, prognosis, assessment, and characterization of pain (e.g., neuropathic pain) and/or inflammation in a subject in need thereof, based upon the expression and/or level of at least one miRNA or exosomal miRNA that is associated with pain and/or inflammation.

In one embodiment, the invention relates to compositions and methods useful for the detection and quantification of miRNAs for predicting a subject's responsiveness to treatment of neuropathic pain, evaluating the efficacy of a treatment of neuropathic pain in a subject, and determining a treatment strategy for a subject. In one embodiment, the compositions and methods of the invention detect and quantify miRNAs for predicting or assessing a subject's responsiveness to administration of an NMDA receptor antagonist as a treatment of neuropathic pain. The NMDA receptor antagonist used for treatment, whose effectiveness is determined using the compositions and methods of the invention, includes, but is not limited to, ketamine, memantine, dizocilpine, APV (AP5), amantadine, dextromethorphan, dextrorphan, AP7, riluzole, tiletamine, midafotel, aptiganel, methoxetamine, ifenprodils, conantokins, and NVP-AAM077. Other compounds and therapies used for treatment, the effectiveness of which is determined using the compositions and methods of the invention, include, but are not limited to, drugs that inhibit TNF-alpha (e.g., Embrel (etanercept), Remicade (infliximab), Thalomid (Thalidomide), Revlimid (lenalidomide)), immune therapies (e.g., intravenous immunoglobulin (IVIG), plasmapheresis, steroids), drugs that act on microglia (e.g., Minocycline, Propentofylline), local anesthetics (e.g., Lidocaine), and alpha-2 adreneric agonists (e.g, Clonidine, Dexmedetomidine).

In one embodiment, the present invention relates to compositions and methods for treating pain (e.g., neuropathic pain) and/or inflammation, including, for example neuropathic pain associated with CRPS. In one embodiment, the present invention provides methods of treating pain (e.g., neuropathic pain) and/or inflammation in a subject comprising administering an effective amount of a therapeutic agent that modulates the activity and/or expression of at least one miRNA or exosomal miRNA associated with responsiveness to the administration of an NMDA receptor antagonist as a treatment of pain (e.g., neuropathic pain) and/or inflammation.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±40% or ±20%, more preferably ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic (e.g., having a wild-type phenotype). Characteristics which are normal or expected for one cell or tissue type, might be abnormal for a different cell or tissue type.

“Antisense,” as used herein, refers to a nucleic acid sequence which is complementary to a target sequence, such as, by way of example, complementary to a target miRNA sequence, including, but not limited to, a mature target miRNA sequence, or a sub-sequence thereof. Typically, an antisense sequence is fully complementary to the target sequence across the full length of the antisense nucleic acid sequence.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “dysregulated” and “dysregulation” as used herein describes a decreased (down-regulated) or increased (up-regulated) level of expression of a miRNA present and detected in a sample of a subject as compared to the level of expression of that miRNA present in a comparator sample, such as a comparator sample of one or more normal, not-at-risk subjects, or from the same subject at a different time point. In some instances, the level of miRNA expression is compared with an average value obtained from more than one not-at-risk individuals. In other instances, the level of miRNA expression is compared with a miRNA level assessed in a sample of one normal, not-at-risk subject.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a comparator.

As used herein, “endogenous” refers to any material from or produced inside an organism, cell, tissue or system.

The term “expression” as used herein is defined as the transcription and/or translation of a particular nucleotide sequence.

“Fragment” as the term is used herein, is a nucleic acid sequence that differs in length (i.e., in the number of nucleotides) from the length of a reference nucleic acid sequence, but retains essential properties of the reference molecule. Preferably, the fragment is at least about 50% of the length of the reference nucleic acid sequence. More preferably, the fragment is at least about 75% of the length of the reference nucleic acid sequence. Even more preferably, the fragment is at least about 95% of the length of the reference nucleic acid sequence.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

As used herein, “hybridization,” “hybridize(s)” or “capable of hybridizing” is understood to mean the forming of a double or triple stranded molecule or a molecule with partial double or triple stranded nature. Complementary sequences in the nucleic acids pair with each other to form a double helix. The resulting double-stranded nucleic acid is a “hybrid.” Hybridization may be between, for example two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids (e.g., LNA compounds). One or both of the nucleic acids may be immobilized on a solid support. Hybridization techniques may be used to detect and isolate specific sequences, measure homology, or define other characteristics of one or both strands. The stability of a hybrid depends on a variety of factors including the length of complementarity, the presence of mismatches within the complementary region, the temperature and the concentration of salt in the reaction or nucleotide modifications in one of the two strands of the hybrid. Hybridizations are usually performed under stringent conditions, for example, at a salt concentration of no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for probe hybridizations. In a particularly preferred embodiment, hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

As used herein, an “instructional material” includes a publication, a recording, a diagram, or any other medium of expression which can be used to communicate the usefulness of a compound, composition, vector, method or delivery system of the invention in the kit for detection of the miRNAs described herein or effecting alleviation of the various diseases or disorders recited herein. Optionally, or alternately, the instructional material can describe one or more methods of detecting miRNA or alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, antisense RNA, siRNA, miRNA, snoRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

“Synthetic mutant” includes any purposefully generated mutant or variant protein or nucleic acid. Such mutants can be generated by, for example, chemical mutagenesis, polymerase chain reaction (PCR) based approaches, or primer-based mutagenesis strategies well known to those skilled in the art.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by the invention include, but are not restricted to, oligonucleotides, nucleic acids, antibodies, cell membrane receptors, monoclonal antibodies and antisera reactive with specific antigenic determinants (such as on viruses, cells or other materials), drugs, peptides, cofactors, lectins, sugars, polysaccharides, cells, cellular membranes, and organelles. Targets are sometimes referred to in the art as anti-probes.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one embodiment, the present invention relates to the discovery that the level of particular microRNAs (miRNAs), including in exosomes, is associated with inflammation or pain. In some embodiments, the miRNA or exosomal miRNA associated with pain and/or inflammation is up-regulated, or expressed at a higher than normal level. In other embodiments, the miRNA or exosomal miRNA associated with pain and/or inflammation is down-regulated, or expressed at a lower than normal level.

In one embodiment, the present invention relates to the discovery that the level of exosomal miRNAs is associated with the responsiveness to treatment of inflammation or pain. In some embodiments, a miRNA associated with responsiveness to treatment of inflammation or pain is up-regulated, or expressed at a higher than normal level. In other embodiments, a miRNA associated with responsiveness to treatment of inflammation or pain is down-regulated, or expressed at a lower than normal level.

In one embodiment, the present invention relates to the discovery that the level of expression of particular miRNAs is associated with the responsiveness to treatment of neuropathic pain. In some embodiments, a miRNA associated with responsiveness to treatment of neuropathic pain is up-regulated, or expressed at a higher than normal level. In other embodiments, a miRNA associated with responsiveness to treatment of neuropathic pain is down-regulated, or expressed at a lower than normal level.

In one embodiment, the present invention relates to the discovery that the level of expression of particular miRNAs is associated with successful treatment of pain and/or inflammation. In some embodiments, a miRNA associated with successful treatment of pain and/or inflammation is up-regulated, or expressed at a higher than normal level. In other embodiments, a miRNA associated successful treatment of pain and/or inflammation is down-regulated, or expressed at a lower than normal level.

In one embodiment, the invention relates to compositions and methods useful for the diagnosis, prognosis, assessment, and characterization of pain and/or inflammation in a subject in need thereof, based upon the expression or level of at least one miRNA or exosomal miRNA that is associated with pain and/or inflammation.

In one embodiment, the invention relates to compositions and methods useful for the detection and quantification of miRNA and/or exosomal miRNA for predicting a subject's responsiveness to treatment of pain and/or inflammation, predicting the subject's outcome to treatment of pain and/or inflammation, evaluating the efficacy of a treatment of pain and/or inflammation in a subject, and determining a treatment strategy for a subject.

In various embodiments, the methods of the invention relate to methods of assessing a subject's risk of having or developing pain and/or inflammation, methods of assessing the severity of a subject's pain and/or inflammation, methods of diagnosing pain and/or inflammation, methods of characterizing pain and/or inflammation, methods of predicting a subject's responsiveness to treatment of pain and/or inflammation, methods of evaluating the efficacy of a treatment of pain and/or inflammation in a subject, and methods of stratifying a subject having pain and/or inflammation in a clinical trial.

In a specific embodiment, the pain is neuropathic pain or pain associated with complex regional pain syndrome (CRPS). In various embodiments of the compositions and methods of the invention described herein, the miRNA associated with pain and/or inflammation, responsiveness to treatment, and/or successful treatment is at least one of hsa-miR-31, hsa-miR-636, hsa-miR-16-1#, hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, hsa-miR-450a, hsa-miR-337-3p, hsa-miR-605, hsa-miR-597, RNU44.A, and hsa-miR-650

In a specific embodiment, the pain is neuropathic pain or pain associated with complex regional pain syndrome (CRPS). In various embodiments of the compositions and methods of the invention described herein, the exosomal miRNA associated with pain and/or inflammation, responsiveness to treatment, and/or successful treatment is at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

It is demonstrated herein that hsa-miR-31 is positively correlated with pain, while hsa-miR-636 and hsa-miR-16-1# are negatively correlated with pain.

It is demonstrated herein that patients who respond to ketamine treatment of neuropathic pain have a higher expression of hsa-miR-197, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a before the initiation of treatment, compared with those who do not respond. Further, patients who respond to ketamine treatment of neuropathic pain have a lower expression of hsa-miR-150 before the initiation of treatment, compared with those who do not respond.

It is demonstrated herein that patients who respond to treatment of neuropathic pain have a higher expression of hsa-miR-650 after treatment, compared with before the initiation of treatment. Further, patients who respond to treatment of neuropathic pain have a lower expression of hsa-miR-337-3p, hsa-miR-605, hsa-miR-597, and RNU44.A after treatment, compared with before the initiation of treatment.

In one embodiment, the method of the invention relates assessing a subject's risk of having or developing neuropathic pain assessing the severity of a subject's neuropathic pain, diagnosing neuropathic pain, characterizing neuropathic pain, and methods of stratifying a subject having neuropathic pain in a clinical trial comprising detecting at least one miRNA associated with neuropathic pain, including at least one of hsa-miR-31, hsa-miR-636, and hsa-miR-16-1#.

In one embodiment, the method of the invention relates to predicting a subject's responsiveness to treatment of neuropathic pain in a subject comprising detecting at least one miRNA associated with the responsiveness of treatment of neuropathic pain, including at least one of hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a.

In one embodiment, the method of the invention relates to evaluating the efficacy of a treatment of neuropathic pain comprising detecting at least one miRNA associated with neuropathic pain or successful treatment of neuropathic pain, including at least one of hsa-miR-31, hsa-miR-636, hsa-miR-16-1#, hsa-miR-337-3p, hsa-miR-605, hsa-miR-597, RNU44.A, and hsa-miR-650.

In one embodiment, the compositions and methods of the invention detect and quantify miRNAs for predicting a subject's responsiveness to administration of an NMDA receptor antagonist as a treatment of neuropathic pain. The NMDA receptor antagonist used for treatment, whose effectiveness is determined using the compositions and methods of the invention, includes, but is not limited to, ketamine, memantine, dizocilpine, phencyclidine, APV (AP5), amantadine, dextromethorphan, dextrorphan, AP7, riluzole, tiletamine, midafotel, aptiganel, methoxetamine, MK-801, ifenprodils, conantokins, and NVP-AAM077. In one embodiment, the method comprises detecting at least one miRNA associated with the responsiveness of administration of an NMDA receptor antagonist as a treatment of neuropathic pain, including at least one of hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a. Other compounds and therapies used for treatment, the effectiveness of which is determined using the compositions and methods of the invention, include, but are not limited to, drugs that inhibit TNF-alpha (e.g., Embrel (etanercept), Remicade (infliximab), Thalomid (Thalidomide), Revlimid (lenalidomide)), immune therapies (e.g., intravenous immunoglobulin (IVIG), plasmapheresis, steroids), drugs that act on microglia (e.g., Minocycline,

Propentofylline), local anesthetics (e.g., Lidocaine), and alpha-2 adreneric agonists (e.g, Clonidine, Dexmedetomidine).

In one embodiment, the present invention relates to compositions and methods for the treatment of neuropathic pain. The present invention is partly based upon the discovery that the expression of hsa-miR-337-3p, hsa-miR-605, hsa-miR-597, RNU44.A, and hsa-miR-650 is modulated after successful ketamine treatment of CRPS. Therefore, in one embodiment, the method comprises administering to a subject in need thereof a composition which modulates the expression and/or activity of at least one of hsa-miR-337-3p, hsa-miR-605, hsa-miR-597, RNU44.A, and hsa-miR-650. In one embodiment, the method comprises inhibiting the expression and/or activity of a miRNA of interest. In one embodiment, the method comprises enhancing the expression and/or activity of a miRNA of interest.

In certain embodiments, a miRNA of interest, as used herein, refers to one or more miRNA associated with neuropathic pain. For example, in certain embodiments, a miRNA of interest is at least one of hsa-miR-31, hsa-miR-636, and hsa-miR-16-1#.

In certain embodiments, a miRNA of interest, as used herein, refers to one or more miRNA associated with responsiveness to treatment of neuropathic pain. For example, in certain embodiments, a miRNA of interest is at least one of hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a.

In certain embodiments, a miRNA of interest, as used herein, refers to one or more miRNA associated with successful treatment of neuropathic pain. For example, in certain embodiments, a miRNA of interest is at least one of hsa-miR-337-3p, hsa-miR-605, hsa-miR-597, RNU44.A, hsa-miR-650.

In one embodiment, the pain is neuropathic pain, such as complex regional pain syndrome (CRPS). In various embodiments of the compositions and methods of the invention described herein, the exosomal miRNA associated with inflammation or pain, responsiveness to treatment, and/or successful treatment is at least one of miR-21#, miR-146b, miR-126-5p, miR-146a, miR-200c, miR-204, miR-212, miR-674, miR-222, miR-342-3p, 27a, miR-878-3p, let-7b, miR-347, miR-155, miR-532-3p, miR-320, miR-146b, miR-24-2#, miR-29c, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

In one embodiment, the method of the invention relates to assessing a subject's risk of having or developing inflammation or pain, assessing the severity of a subject's inflammation or pain, diagnosing inflammation or pain, characterizing inflammation or pain, and methods of stratifying a subject having inflammation or pain in a clinical trial comprising detecting at least one exosomal miRNA associated with inflammation or pain, including at least one of miR-21#, miR-146b, miR-126-5p, miR-146a, miR-200c, miR-204, miR-212, miR-674, miR-222, miR-342-3p, miR-27a, miR-878-3p, let-7b, miR-347, miR-155, miR-532-3p, miR-320, miR-146b, miR-24-2#, miR-29c, miR-326, miR-720, miR 93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

In certain embodiments, the exosomal miRNA of interest, as used herein, refers to one or more exosomal miRNA associated with inflammation or pain. For example, in certain embodiments, a miRNA of interest is at least one of miR-21#, miR-146b, miR-126-5p, miR-146a, miR-200c, miR-204, miR-212, miR-674, miR-222, miR-342-3p, miR-27a, miR-878-3p, let-7b, miR-347, miR-155, miR-532-3p, miR-320, miR-146b, miR-24-2#, miR-29c, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

### Assays

In one embodiment, the present invention relates to the discovery that the expression or level of particular miRNAs or exosomal miRNAs is associated with the presence, development, progression and severity of pain and/or inflammation. In one embodiment, the present invention relates to the discovery that the expression or level of particular miRNAs or exosomal miRNAs is associated with the responsiveness to the treatment of pain and/or inflammation. In one embodiment, the present invention relates to the discovery that the expression or level of particular miRNAs or exosomal miRNAs is associated with successful treatment of pain and/or inflammation.

In a particular embodiment, the invention relates to compositions and methods of detecting and quantifying particular miRNAs or exosomal miRNAs associated with the responsiveness of administration of a treatment of pain and/or inflammation (e.g., an NMDA receptor antagonist). In various embodiments, the invention relates to a genetic screening assay of a subject to determine the level of expression of at least one miRNA or exosomal miRNA of interest in the subject.

The present invention provides methods of assessing the level of at least one miRNA or exosomal miRNA of interest. In certain embodiments, the invention provides methods of diagnosing a subject as having, or as being at risk of developing, pain and/or inflammation based upon the level of expression of at least one miRNA or exosomal miRNA associated with pain and/or inflammation. In certain embodiments, the invention provides methods of predicting or assessing a subject's response to treatment of pain and/or inflammation, or determining an appropriate treatment strategy of pain and/or inflammation, based upon the level of expression of at least one miRNA or exosomal miRNA associated with the responsiveness of treatment of pain and/or inflammation. In one embodiment, the treatment of neuropathic pain comprises administration of at least one NMDA receptor antagonist. In some embodiments, the diagnostic assays described herein are in vitro assays. In other embodiments, the diagnostic assays described herein are in vivo assays.

In one embodiment, the method of the invention is a diagnostic assay for assessing the presence, development, progression and severity of pain and/or inflammation in a subject in need thereof, by determining whether the level of at least one miRNA or exosomal miRNA associated with pain and/or inflammation is increased in a biological sample obtained from the subject. In various embodiments, to determine whether the level of the at least one miRNA or exosomal miRNA associated with pain and/or inflammation is increased or decreased in a biological sample obtained from the subject, the level of the at least one miRNA or exosomal miRNA is compared with the level of at least one comparator control, such as a positive control, a negative control, a normal control, a wild-type control, a historical control, a historical norm, or the level of another reference molecule in the biological sample. In some embodiments, the diagnostic assay of the invention is an in vitro assay. In other embodiments, the diagnostic assay of the invention is an in vivo assay. The miRNA identified by the assay can be any miRNA that is associated with neuropathic pain. In some embodiments, the miRNA is at least one of hsa-miR-31, hsa-miR-636, and hsa-miR-16-1#. The exosomal miRNA identified by the assay can be any exosomal miRNA that is associated with inflammation or pain. In some embodiments, the exosomal miRNA is at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, and miR-199a-3p. In other embodiments, the exosomal miRNA is at least one of miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201. In various embodiments of the invention, the at least one miRNA or exosomal miRNA associated with pain and/or inflammation is at least two miRNA or exosomal miRNAs, at least three miRNA or exosomal miRNAs, at least four miRNA or exosomal miRNAs, at least five miRNA or exosomal miRNAs, at least six miRNA or exosomal miRNAs, at least seven miRNA or exosomal miRNAs, at least eight miRNA or exosomal miRNAs, at least nine miRNA or exosomal miRNAs, or at least ten miRNA or exosomal miRNAs. The results of the diagnostic assay can be used alone, or in combination with other information from the subject, or other information from the biological sample obtained from the subject.

In one embodiment, the method of the invention is a diagnostic assay for predicting a subject's responsiveness to a treatment of pain and/or inflammation and determining an appropriate treatment strategy for pain and/or inflammation, by determining whether the level of at least one miRNA or exosomal miRNA associated with responsiveness to a treatment of pain and/or inflammation is increased in a biological sample obtained from the subject. In one embodiment, the treatment of neuropathic pain, whose effectiveness in a particular subject is assessed by way of the present invention, comprises administration of at least one NMDA receptor antagonist. In various embodiments, to determine whether the level of the at least one miRNA or exosomal miRNA associated with the responsiveness of a treatment of pain and/or inflammation is increased or decreased in a biological sample obtained from the subject, the level of the at least one miRNA or exosomal miRNA is compared with the level of at least one comparator control, such as a positive control, a negative control, a normal control, a wild-type control, a historical control, a historical norm, or the level of another reference molecule in the biological sample. In one embodiment, the comparator control comprises the level of the at least one miRNA or exosomal miRNA in a control subject known to respond to treatment. In one embodiment, the comparator control comprises the level of the at least one miRNA or exosomal miRNA in a control subject known to not respond to treatment. In some embodiments, the diagnostic assay of the invention is an in vitro assay. In other embodiments, the diagnostic assay of the invention is an in vivo assay. The miRNA identified by the assay can be any miRNA or exosomal miRNA that is associated with responsiveness to a treatment of pain and/or inflammation. In some embodiments, the miRNA is at least one of hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a. In some embodiments, the exosomal miRNA is at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, and miR-199a-3p. In other embodiments, the exosomal miRNA is at least one of miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201. In various embodiments of the invention, the at least one miRNA or exosomal miRNA associated with responsiveness to a treatment of pain and/or inflammation is at least two miRNA or exosomal miRNAs, at least three miRNA or exosomal miRNAs, at least four miRNA or exosomal miRNAs, at least five miRNA or exosomal miRNAs, at least six miRNA or exosomal miRNAs, at least seven miRNA or exosomal miRNAs, at least eight miRNA or exosomal miRNAs, at least nine miRNA or exosomal miRNAs, at least ten miRNA or exosomal miRNAs. The results of the diagnostic assay can be used alone, or in combination with other information from the subject, or other information from the biological sample obtained from the subject.

In another embodiment, the method of the invention is an assay for monitoring the effectiveness of a treatment administered to a subject in need thereof, by determining whether the level of at least one miRNA or exosomal miRNA associated with pain and/or inflammation or successful treatment of pain and/or inflammation is increased in a biological sample obtained from the subject. In various embodiments, to determine whether the level of the at least one miRNA or exosomal miRNA associated with pain and/or inflammation or successful treatment of pain and/or inflammation is increased in a biological sample obtained from the subject, the level of the at least one miRNA or exosomal miRNA is compared with the level of at least one comparator control, such as a positive control, a negative control, a normal control, a wild-type control, a historical control, a historical norm, or the level of another reference molecule in the biological sample. In one embodiment, the comparator control comprises the level of the at least one miRNA or exosomal miRNA in a control subject known to respond to treatment. In one embodiment, the comparator control comprises the level of the at least one miRNA or exosomal miRNA in a control subject known to not respond to treatment. In some embodiments, the diagnostic assay of the invention is an in vitro assay. In other embodiments, the diagnostic assay of the invention is an in vivo assay. ThemiRNA or exosomal miRNA identified by the assay can be any miRNA or exosomal miRNA that is associated with neuropathic pain or successful treatment of pain and/or inflammation. In some embodiments, the miRNA is at least one of hsa-miR-31, hsa-miR-636, hsa-miR-16-1#, hsa-miR-337-3p, hsa-miR-605, hsa-miR-597, RNU44.A, hsa-miR-650. The miRNA or exosomal miRNA identified by the assay can be any miRNA or exosomal miRNA that is associated with inflammation or pain or successful treatment of pain and/or inflammation. In some embodiments, the exosomal miRNA is at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201. In various embodiments of the invention, the at least one miRNA or exosomal miRNA associated with pain and/or inflammation or successful treatment of pain and/or inflammation is at least two miRNA or exosomal miRNAs, at least three miRNA or exosomal miRNAs, at least four miRNA or exosomal miRNAs, at least five miRNA or exosomal miRNAs, at least six miRNA or exosomal miRNAs, at least seven miRNA or exosomal miRNAs, at least eight miRNA or exosomal miRNAs, at least nine miRNA or exosomal miRNAs, or at least ten miRNA or exosomal miRNAs. The results of the diagnostic assay can be used alone, or in combination with other information from the subject, or other information from the biological sample obtained from the subject.

In a further embodiment, the method of the invention is an assay for assessing pain and/or inflammation in a subject for the purpose of stratifying the subject for assignment in a clinical trial, by determining whether the level of at least one miRNA or exosomal miRNA associated with pain and/or inflammation or responsiveness to treatment of pain and/or inflammation is increased in a biological sample obtained from the subject. In various embodiments, to determine whether the level of the at least one miRNA or exosomal miRNA associated with pain and/or inflammation or responsiveness to treatment of pain and/or inflammation is increased in a biological sample obtained from the subject, the level of the at least one miRNA or exosomal miRNA is compared with the level of at least one comparator control, such as a positive control, a negative control, a normal control, a wild-type control, a historical control, a historical norm, or the level of another reference molecule in the biological sample. In one embodiment, the comparator control comprises the level of the at least one miRNA or exosomal miRNA in a control subject known to respond to treatment. In one embodiment, the comparator control comprises the level of the at least one miRNA or exosomal miRNA in a control subject known to not respond to treatment. In some embodiments, the diagnostic assay of the invention is an in vitro assay. In other embodiments, the diagnostic assay of the invention is an in vivo assay. ThemiRNA or exosomal miRNA identified by the assay can be any miRNA or exosomal miRNA that is associated with pain and/or inflammation or responsiveness to treatment of pain and/or inflammation. The subject can be stratified into a clinical trial based upon the information obtained from the assay, including, but not limited to, the severity of pain and/or inflammation, or the expression level of at least one miRNA or exosomal miRNA associated with neuropathic pain. In some embodiments, the miRNA is at least one of hsa-miR-31, hsa-miR-636, hsa-miR-16-1#hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a. In some embodiments, the exosomal miRNA is at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, and miR-199a-3p. In other embodiments, the exosomal miRNA is at least one of miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201. In various embodiments of the invention, the at least one miRNA or exosomal miRNA associated with pain and/or inflammation or responsiveness to treatment of pain and/or inflammation is at least two miRNA or exosomal miRNAs, at least three miRNA or exosomal miRNAs, at least four miRNA or exosomal miRNAs, at least five miRNA or exosomal miRNAs, at least six miRNA or exosomal miRNAs, at least seven miRNA or exosomal miRNAs, at least eight miRNA or exosomal miRNAs, at least nine miRNA or exosomal miRNAs, or at least ten miRNA or exosomal miRNAs. The results of the diagnostic assay can be used alone, or in combination with other information from the subject, or other information from the biological sample obtained from the subject.

In various embodiments of the assays of the invention, the level of the at least one miRNA or exosomal miRNA of interest is determined to be up-regulated when the level of the at least one miRNA or exosomal miRNA is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator control.

In other various embodiments of the assays of the invention, the level of miRNA or exosomal miRNA of interest is determined to be down-regulated when the level of the at least one miRNA or exosomal miRNA is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator control.

In the assay methods of the invention, a test biological sample from a subject is assessed for the expression level of at least one miRNA or exosomal miRNA of interest. The test biological sample can be an in vitro sample or an in vivo sample. In various embodiments, the subject is a human subject, and may be of any race, sex and age. Representative subjects include those who are suspected of having pain and/or inflammation, those who have been diagnosed with pain and/or inflammation, those whose have pain and/or inflammation, those who have had pain and/or inflammation, those undergoing treatment of pain and/or inflammation, those who have had treatment of pain and/or inflammation, those being evaluated for potential treatment of pain and/or inflammation, those who at risk of a recurrence of pain and/or inflammation, and those who are at risk of developing pain and/or inflammation.

In some embodiments, a binding molecule that specifically binds to a miRNA or exosomal miRNA of interest is used to detect the miRNA or exosomal miRNA. In certain embodiments, the binding molecule is used in vivo for the diagnosis of pain and/or inflammation. In some embodiments, the binding molecule is nucleic acid that hybridizes with a miRNA or exosomal miRNA of interest.

In one embodiment, the test sample is a sample containing at least a fragment of a nucleic acid comprising a miRNA or exosomal miRNA of interest. The term, “fragment,” as used herein, indicates that the portion of a nucleic acid (e.g., DNA, mRNA or cDNA) that is sufficient to identify it as comprising a miRNA or exosomal miRNA of interest.

In some embodiments, the test sample is prepared from a biological sample of the subject. The biological sample can be a sample from any source which contains a nucleic acid comprising a miRNA or exosomal miRNA of interest, such as a body fluid (e.g., blood, plasma, serum, saliva, urine, etc.), or a tissue, or an exosome, or a cell, or a combination thereof. A biological sample can be obtained by appropriate methods, such as, by way of examples, biopsy or fluid draw. The biological sample can be used as the test sample; alternatively, the biological sample can be processed to enhance access to polypeptides, nucleic acids, or copies of nucleic acids (e.g., copies of nucleic acids comprising a miRNA or exosomal miRNA of interest), and the processed biological sample can then be used as the test sample. For example, in various embodiments, nucleic acid is prepared from a biological sample, for use in the methods. Alternatively or in addition, if desired, an amplification method can be used to amplify nucleic acids comprising all or a fragment of a nucleic acid in a biological sample, for use as the test sample in the assessment of the expression level of a miRNA or exosomal miRNA of interest.

The test sample is assessed to determine the level of expression of at least one miRNA or exosomal miRNA of interest present in the nucleic acid of the subject. In general, detecting an miRNA or exosomal miRNA may be carried out by determining the presence or absence of a nucleic acid containing an miRNA or exosomal miRNA of interest in the test sample.

In some embodiments, hybridization methods, such as Northern analysis, or in situ hybridizations, can be used (see Current Protocols in Molecular Biology, 2012, Ausubel, F. et al., eds., John Wiley & Sons, including all supplements). For example, the presence of an miRNA or exosomal miRNA of interest can be indicated by hybridization to a nucleic acid probe. A “nucleic acid probe,” as used herein, can be a nucleic acid probe, such as a DNA probe or an RNA probe. For representative examples of use of nucleic acid probes, see, for example, U.S. Pat. Nos. 5,288,611 and 4,851,330.

To detect at least one miRNA or exosomal miRNA of interest, a hybridization sample is formed by contacting the test sample with at least one nucleic acid probe. A preferred probe for detecting miRNA or exosomal miRNA is a labeled nucleic acid probe capable of hybridizing to miRNA or exosomal miRNA. The nucleic acid probe can be, for example, a full-length nucleic acid molecule, or a portion thereof, such as an oligonucleotide of at least 10, 15, or 25 nucleotides in length and sufficient to specifically hybridize under stringent conditions to appropriate miRNA or exosomal miRNA. The hybridization sample is maintained under conditions which are sufficient to allow specific hybridization of the nucleic acid probe to an miRNA or exosomal miRNA target of interest. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and an miRNA or exosomal miRNA in the test sample, the sequence that is present in the nucleic acid probe is also present in the miRNA or exosomal miRNA of the subject. More than one nucleic acid probe can also be used concurrently in this method. Specific hybridization of any one of the nucleic acid probes is indicative of the presence of the miRNA or exosomal miRNA of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising at least one miRNA or exosomal miRNA of interest. Hybridization of the PNA probe to a nucleic acid sequence is indicative of the presence of an miRNA or exosomal miRNA of interest.

Direct sequence analysis can also be used to detect miRNA and/or exosomal miRNA of interest. A sample comprising nucleic acid can be used, and PCR or other appropriate methods can be used to amplify all or a fragment of the nucleic acid, and/or its flanking sequences, if desired.

In another embodiment, arrays of oligonucleotide probes that are complementary to target nucleic acid sequences from a subject can be used to detect, identify and quantify miRNA and/or exosomal miRNA of interest. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a sample containing miRNA and/or exosomal miRNA is hybridized with the array and scanned for miRNA and/or exosomal miRNA. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein.

In brief, a target miRNA or exosomal miRNA sequence is amplified by well-known amplification techniques, e.g., RT, PCR. Typically, this involves the use of primer sequences that are complementary to the target miRNA or exosomal miRNA. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect miRNA and/or exosomal miRNA of interest. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., 1981, Proc. Natl. Acad. Sci. USA 86:232-236), mobility shift analysis (Orita et al., 1989, Proc. Natl. Acad. Sci. USA 86:2766-2770; Rosenbaum and Reissner, 1987, Biophys. Chem. 265:1275; 1991, Keen et al., Trends Genet. 7:5); RNase protection assays (Myers, et al., 1985, Science 230:1242); Luminex xMAP™ technology; high-throughput sequencing (HTS) (Gundry and Vijg, 2011, Mutat Res, doi:10.1016/j.mrfmmm 2011.10.001); next-generation sequencing (NGS) (Voelkerding et al., 2009, Clinical Chemistry 55:641-658; Su et al., 2011, Expert Rev Mol Diagn. 11:333-343; Ji and Myllykangas, 2011, Biotechnol Genet Eng Rev 27:135-158); and/or ion semiconductor sequencing (Rusk, 2011, Nature Methods doi:10.1038/nmeth.f.330; Rothberg et al., 2011, Nature 475:348-352). These and other methods, alone or in combination, can be used to detect and quantity of at least one miRNA or exosomal miRNA of interest, in a biological sample obtained from a subject. In one embodiment of the invention, the methods of assessing a biological sample to detect and quantify an miRNA or exosomal miRNA of interest, as described herein, are used to diagnose, prognosticate, assess and characterize pain and/or inflammation in a subject in need thereof.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents. Nucleic acids can be obtained from the biological sample using known techniques.

Nucleic acid herein includes RNA, including mRNA, miRNA, exosomal miRNA, etc. The nucleic acid can be double-stranded or single-stranded (i.e., a sense or an antisense single strand) and can be complementary to a nucleic acid encoding a polypeptide. The nucleic acid content may also be obtained from an extraction performed on a fresh or fixed biological sample.

There are many methods known in the art for the detection of specific nucleic acid sequences and new methods are continually reported. A great majority of the known specific nucleic acid detection methods utilize nucleic acid probes in specific hybridization reactions.

In the Northern blot, the nucleic acid probe is preferably labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a desired region of the target nucleic acid of interest is hybridized in a sample, subjected to depolymerizing conditions, and the sample is treated with an ATP/luciferase system, which will luminesce if the nucleic sequence is present. In quantitative Northern blotting, levels of the polymorphic nucleic acid can be compared to wild-type levels of the nucleic acid.

A further process for the detection of hybridized nucleic acid takes advantage of the polymerase chain reaction (PCR). The PCR process is well known in the art (U.S. Pat. No. 4,683,195, No. 4,683,202, and No. 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

Nucleic acid amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable polymerase. The extension product is then denatured from the target sequence by heating, and the process is repeated. Successive cycling of this procedure on both strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

Stem-loop RT-PCR is a PCR method that is useful in the methods of the invention to amplify and quantify miRNA and/or exosomal miRNA of interest (See Caifu et al., 2005, Nucleic Acids Research 33:e179; Mestdagh et al., 2008, Nucleic Acids Research 36:e143; Varkonyi-Gasic et al., 2011, Methods Mol Biol. 744:145-57). Briefly, the method includes two steps: RT and real-time PCR. First, a stem-loop RT primer is hybridized to an miRNA or exosomal miRNA molecule and then reverse transcribed with a reverse transcriptase. Then, the RT products are quantified using conventional real-time PCR.

The expression specifically hybridizing in stringent conditions refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the template nucleic acid under conditions of stringency that prevent non-specific binding but permit binding of this template nucleic acid which has a significant level of homology with the probe or primer.

Among the conditions of stringency is the melting temperature (Tm) for the amplification step using the set of primers, which is in the range of about 50° C. to about 95° C. Typical hybridization and washing stringency conditions depend in part on the size (i.e., number of nucleotides in length) of the template nucleic acid or the oligonucleotide probe, the base composition and monovalent and divalent cation concentrations (Ausubel et al., 1994, eds Current Protocols in Molecular Biology).

In a preferred embodiment, the process for determining the quantitative and qualitative profile according to the present invention is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 50° C. to 95° C. Preferably, the Tm for any one of the hydrolysis-probes of the present invention is in the range of about 55° C. to about 80° C. Most preferably, the Tm applied for any one of the hydrolysis-probes of the present invention is about 75° C.

In another preferred embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a nucleic acid sequence of the miRNA or exosomal miRNA of interest, and more particularly the primer includes 12 or more contiguous nucleotides substantially complementary to the sequence of the miRNA or exosomal miRNA of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target nucleotide sequence In another aspect, the length of the primer can vary in length, preferably about 15 to 28 nucleotides in length (e.g., 15, 16, 17, 18, 19, 20, 21, 22, 23, 24, 25, 26, 27 or 28 nucleotides in length).

### Compositions

One aspect of this invention relates to an agent, hereinafter referred to as an agent of the invention, characterized by its ability to detect one or more miRNA or exosomal miRNA of interest. Non-limiting examples of an agent able to detect one or more miRNA or exosomal miRNA of interest include an antibody, an aptamer, a molecular probe, peptide, peptidomimetic, small molecule, and conjugates thereof.

Another aspect of this invention relates to a therapeutic agent characterized by its ability to modulate the expression and/or activity one or more miRNA or exosomal miRNA of interest. For example, as described elsewhere herein, the present invention is partly based upon the discovery of five miRNAs whose expression is altered after successful treatment of CRPS. In one embodiment, an agent of the invention has the ability to modulate the expression of at least one of hsa-miR-337-3p, hsa-miR-605, hsa-miR-597, RNU44.A, and hsa-miR-650. It was found that hsa-miR-337-3p, hsa-miR-605, hsa-miR-597, and RNU44.A were downregulated after successful treatment, while hsa-miR-650 was upregulated. In one embodiment, the therapeutic agent of the invention modulates the level, activity and/or expression of at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

Therefore, in one embodiment, the therapeutic agent of the invention inhibits the activity and/or expression of at least one of hsa-miR-337-3p, hsa-miR-605, hsa-miR-597, and RNU44.A. In one embodiment, the therapeutic agent of the invention enhances the activity and/or expression of hsa-miR-650. In one embodiment, the composition of the invention comprises both a therapeutic agent that inhibits at least one of hsa-miR-337-3p, hsa-miR-605, hsa-miR-597, and RNU44.A, and a therapeutic agent that enhances the activity and/or expression of hsa-miR-650.

In certain embodiments, the therapeutic agent can be used to treat pain and/or inflammation, including, for example, pain associated with CRPS. The agent, which can be identified and evaluated according to the present invention, can be any agent including but not limited to small molecules, antibodies, antibody fragments, peptides, peptidomimetics, nucleic acids, antisense molecules, ribozymes, triple-helix molecules, miRNA, exosomal miRNA, double stranded RNA etc., which modulates the level, expression and/or the activity of one or more miRNA or exosomal miRNA of interest. In one embodiment, the agent of the invention inhibits the level, expression and/or activity of one or more miRNA or exosomal miRNA of interest. In another embodiment, the agent of the invention promotes or enhances the level, expression and/or activity of one or more miRNA or exosomal miRNA of interest. In certain embodiments, the composition comprises a pharmaceutical composition comprising a therapeutic agent which modulates the activity and/or expression of a miRNA of interest, including, but not limited to hsa-miR-337-3p, hsa-miR-605, hsa-597, RNU44.A, and hsa-miR-650. In another embodiment, the agent of the invention promotes or enhances the level, expression and/or activity of one or more miRNA or exosomal miRNA of interest. In certain embodiments, the composition comprises a pharmaceutical composition comprising a therapeutic agent which modulates the level, activity and/or expression of an exosomal miRNA of interest, including, but not limited to miR-21#, miR-146b, 126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

### Methods of Treatment

In one embodiment, the present invention comprises a method of treating pain and/or inflammation in a subject. In certain embodiments, the invention comprises a method of treating CRPS in a subject. In certain embodiments, the invention comprises a method of treating fibromyalgia or other chronic pain conditions. In one embodiment, the method comprises administering an effective amount of a composition which modulates the level, activity and/or expression of at least one miRNA or exosomal miRNA of interest, including, but not limited to hsa-miR-337-3p, hsa-miR-605, hsa-miR-597, RNU44.A, and hsa-miR-650. In one embodiment, the method comprises administering an effective amount of a composition which modulates the level, activity and/or expression of at least one miRNA or exosomal miRNA of interest, including, but not limited to miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201. In one embodiment, the method comprises administering an effective amount of a composition which inhibits the level, activity and/or expression of an miRNA or exosomal miRNA of interest. In one embodiment, the method comprises administering an effective amount of a composition which enhances the activity and/or expression of an miRNA or exosomal miRNA of interest.

In one embodiment, the method comprises administering an effective amount of a composition which inhibits the activity and/or expression of at least one of hsa-miR-337-3p, hsa-miR-605, hsa-miR-597, and RNU44.A. In one embodiment, the method comprises administering an effective amount of a composition which enhances the activity and/or expression of hsa-miR-650.

In one embodiment, the method comprises administering an effective amount of a composition which modulates the level, activity and/or expression of at least one of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

In one embodiment, the method of the invention comprises administering a therapeutic agent, as described elsewhere herein, to a subject in need thereof. A subject in need thereof includes those who are suspected of having pain and/or inflammation, those who have been diagnosed with pain and/or inflammation, those whose have pain and/or inflammation, those who have had pain and/or inflammation, those undergoing treatment of pain and/or inflammation, those who have had treatment of pain and/or inflammation, those being evaluated for potential treatment of pain and/or inflammation, those who at risk of a recurrence of pain and/or inflammation, and those who are at risk of developing pain and/or inflammation. In certain embodiments, the subject is a mammal. In one embodiment, the subject is a human.

The method of the invention comprises administration of a therapeutic agent, as described elsewhere herein, by any suitable method known in the art. For example, in certain embodiments, the therapeutic agent is delivered to a patient subcutaneously, intradermally, intratumorally, intranodally, intramedullary, intramuscularly, intravenously, or intraperitoneally. The dosage of the above treatments to be administered to a patient will vary with the precise nature of the condition being treated and the recipient of the treatment. The scaling of dosages for human administration can be performed according to art-accepted practices. The quantity and frequency of administration will be determined by such factors as the condition of the patient, and the type and severity of the patient's disease, although appropriate dosages may be determined by clinical trials.

In one embodiment, the method comprises administering a therapeutic agent, as described herein, in combination with one or more additional therapies of pain (e.g., neuropathic pain and/or inflammation). The one or more additional therapies of pain and/or inflammation may be one or more additional therapeutic agents, including, but not limited to, anesthetics, analgesics, NMDA receptor antagonists, opioids, antiepileptics, antidepressants, and the like. In another embodiment, the one or more additional therapies of pain and/or inflammation include non-pharmaceutical based therapies, including, but not limited to electric stimulation, counseling, physical therapy, psychotherapy, biofeedback, relaxation techniques and the like.

### Kits

The present invention also pertains to kits useful in the methods of the invention. Such kits comprise components useful in any of the methods described herein, including for example, hybridization probes or primers (e.g., labeled probes or primers), reagents for detection of labeled molecules, oligonucleotide arrays, restriction enzymes, antibodies, allele-specific oligonucleotides, means for amplification of a subject's nucleic acids, means for reverse transcribing a subject's RNA, means for analyzing a subject's nucleic acid sequence, and instructional materials. For example, in one embodiment, the kit comprises components useful for the detection and quantification of at least one miRNA or exosomal miRNA of interest. In a preferred embodiment of the invention, the kit comprises components for detecting one or more of the miRNAs or exosomal miRNAs of interest as elsewhere described herein. In one embodiment, the kit comprises a therapeutic agent described herein and optionally components for administering the therapeutic agent to a subject in need thereof.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Treatment Induced MicroRNA Modulation in Complex Regional Pain Syndrome

The experiments described herein were conducted in order to determine the utility of miRNAs as biomarkers for CRPS in both the effectiveness of a treatment and the severity and progression of individual cases. As presented herein, the changes in levels of 758 miRNAs were examined in blood of 19 CRPS patients before and after ketamine treatment, using Taqman low-density array cards. These results were compared to the patients' reported changes in pain. These biomarkers are a valuable tool for stratifying patients in clinical trials and in assisting physicians in choosing treatment options.

miRNAs are approximately 22 nucleotide noncoding RNAs that regulate gene expression. miRNAs bind to 3′ untranslated regions of specific messenger RNA (mRNA) to induce cleavage of mRNA or translational repression. Stable miRNAs are present in all body fluids (Weber et al., 2010, Clinical Chemistry, 56(11): 1733-1741) and miRNA alterations have been observed in diseases such as cancer and neurological diseases. Due to their stability and prevalence in many body fluids, miRNAs hold immense promise as more precise and economical diagnostic tools.

The materials and methods employed in these experiments are now described.

### Patient Study

All subjects were enrolled after giving informed consent as approved by the Drexel University Institutional Review Board.

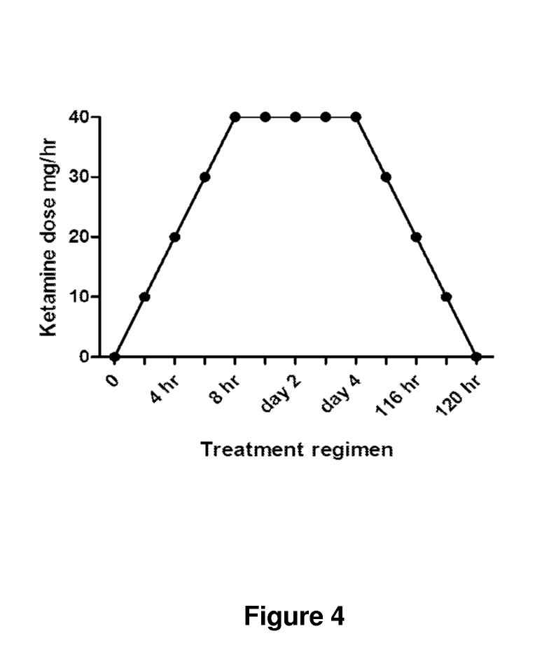

Ketamine Treatment Ketamine treatment was given to the subjects by sub-anesthetic continuous intravenous administration. Infusion started at a rate of 10 mg/hr and increased in steps of 10 mg/hr at every 2 hours to a maximum of 40 mg/hr (see FIG. 4). Blood samples were collected from CRPS patients before and after ketamine treatment.
miRNA Analysis

Total RNA was isolated from blood samples using mirVana kit (Applied Biosystems) and cDNA synthesis was performed. Levels of 758 miRNAs were analyzed by using a Taqman low-density array (TLDA) card.

The results of the experiments are now described.

### Patient Profiles

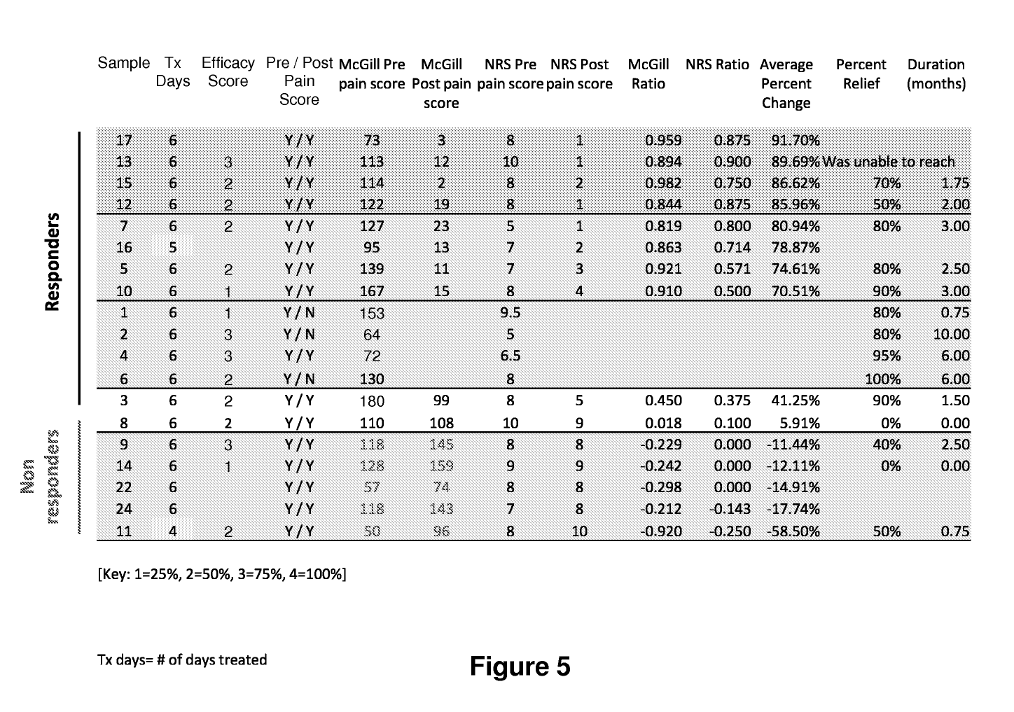

Patients reported changes in pain before and after treatment were determined using the McGill Pain Questionnaire, a series of 22 questions where each question is ranked on a scale of 1-10. The maximum score possible is a 220. Results of the questionnaire are presented in Table 1. A subset of patients did not experience pain relief and are considered as poor responders (shown in red; Patient IDs 8, 9, 14, 22 and 11). FIG. 5 depicts more detailed metrics of the change in pain in the patients of the study.

TABLE 1

| | Average percent | Pain relief reported | Pain relief |
|---|---|---|---|
| Patient ID | decrease in pain | by the patient | in months |
| 17 | 91.70% | — | — |
| 13 | 89.69% | — | — |
| 15 | 86.62% | 70% | 1.75 |
| 12 | 85.96% | 50% | 2 |
| 7 | 80.94% | 80% | 3 |
| 16 | 78.87% | — | — |
| 5 | 74.61% | 80% | 2.5 |
| 10 | 70.51% | 90% | 3 |
| 3 | 41.25% | 90% | 1.5 |
| 8 | 5.91% | 0% | 0 |
| 9 | −11.44% | 40% | 2.5 |
| 14 | −12.11% | 0% | 0 |
| 22 | −14.91% | — | — |
| 11 | −58.50% | 50% | 0.75 |

Correlation of Pain Score and miRNAs Before Treatment

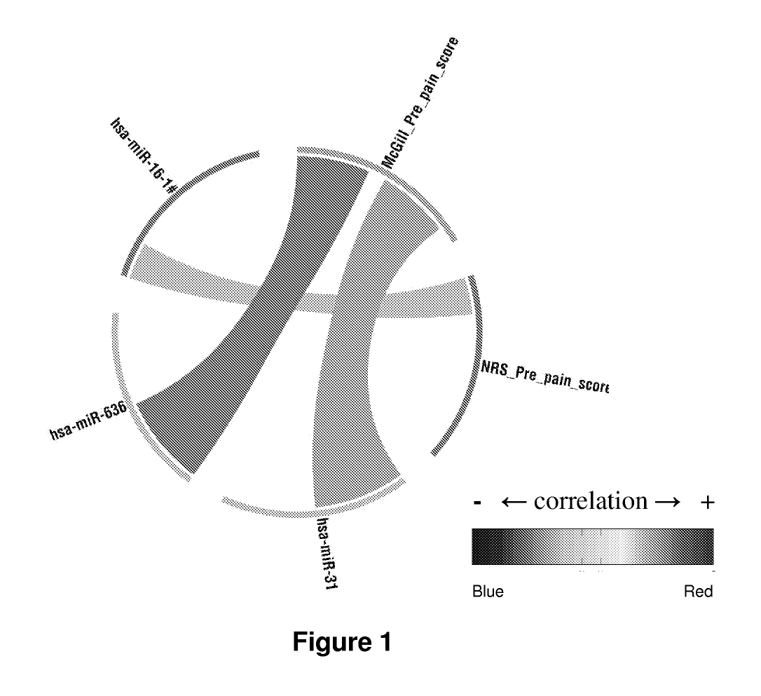

A Circos diagram was created (FIG. 1) which illustrates the correlation of pain score and miRNAs before treatment. Three miRNAs with strongest correlation (represented by thickness of the band) are shown. Negative correlations are shown in blue and positive correlations are represented in red. As shown in FIG. 1, has-miR-31 is positively correlated with McGill Pain Score (before treatment), while hsa-miR-636 is negatively correlated with McGill Pain Score (before treatment). Further, has-miR-16-1# was negatively correlated with Numeric Rating Scale (NRS) Pain Score (before treatment).

Differentially Expressed miRNAs in Blood from Responders Vs. Non-Responder CRPS Patients Before Ketamine Treatment

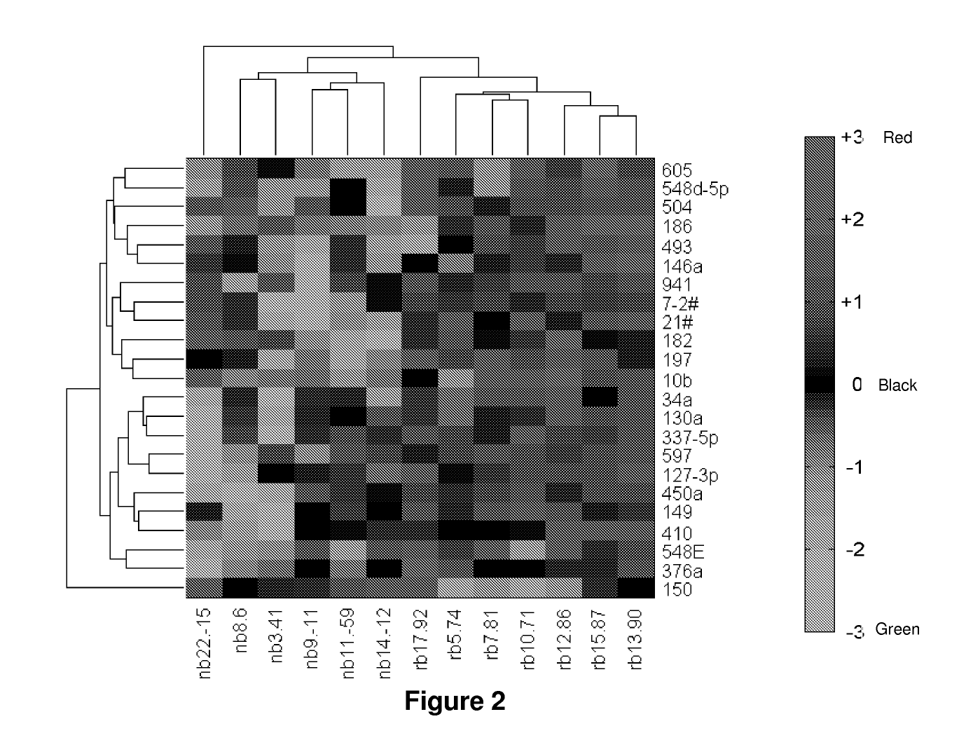

Analysis of miRNAs in blood samples from the subjects revealed the presence of 23 different miRNAs that were differentially expressed among responders and non-responders in blood samples taken prior to ketamine treatment. FIG. 2 is a clustergram of the samples demonstrating the significant differentially expressed miRNAs in responders and non-responders before treatment (Red, high; black, average; green, low). Table 2 lists the fold changes and p values of significantly altered miRNAs (data sorted based on p value). A positive fold change indicates a higher expression of the miRNA in responders, while a negative fold change indicates a lower expression of the miRNA in responders. The statistical significance was calculated using paired 2-tailed t-tests on the miRNA expressions in good and poor responders before treatment.

TABLE 2

| miRNA | Fold change | P-value |
|---|---|---|
| hsa-miR-197 | 1.81 | 0.003 |
| hsa-miR-150 | −1.35 | 0.018 |
| hsa-miR-186 | 2.02 | 0.018 |
| hsa-miR-10b | 28.12 | 0.019 |
| hsa-miR-605 | 28.02 | 0.019 |
| hsa-miR-597 | 9.43 | 0.021 |
| hsa-miR-410 | 6.55 | 0.022 |
| hsa-miR-337-5p | 9.64 | 0.026 |
| hsa-miR-548d-5p | 20.50 | 0.028 |
| hsa-miR-548E | 13.82 | 0.030 |
| hsa-miR-21# | 9.33 | 0.030 |
| hsa-miR-7-2# | 15.52 | 0.030 |
| hsa-miR-182 | 2.61 | 0.031 |
| hsa-miR-34a | 25.87 | 0.035 |
| hsa-miR-376a | 5.04 | 0.036 |
| hsa-miR-149 | 6.93 | 0.037 |
| hsa-miR-504 | 7.99 | 0.040 |
| hsa-miR-941 | 12.13 | 0.041 |
| hsa-miR-493 | 21.41 | 0.042 |
| hsa-miR-146a | 2.12 | 0.045 |
| hsa-miR-127-3p | 6.16 | 0.047 |
| hsa-miR-130a | 5.63 | 0.048 |
| hsa-miR-450a | 20.44 | 0.048 |

Differentially Expressed miRNA Responders Before and after Ketamine Treatments

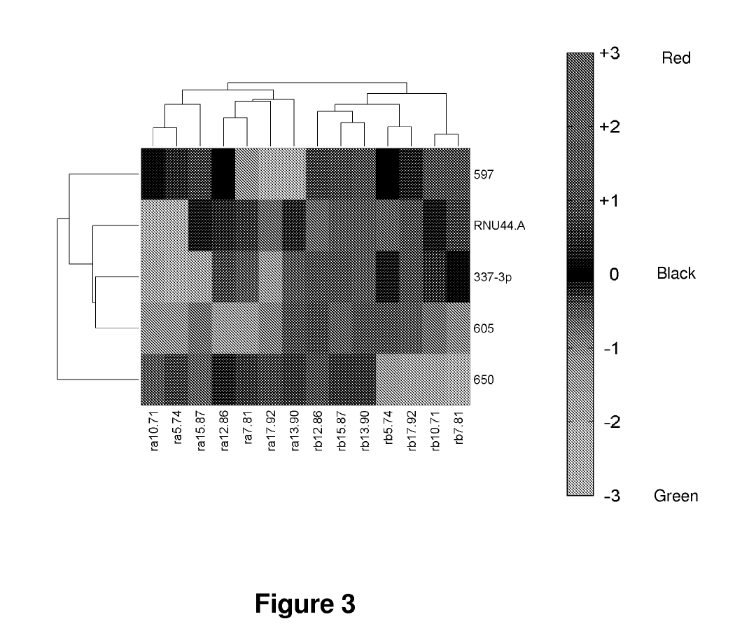

Analysis of miRNAs in blood samples from the subjects revealed the presence of 5 miRNAs that were differentially expressed in the blood samples of responders before and after treatment. FIG. 3 is a clustergram of the samples depicting the differentially expressed miRNAs in responders before and after treatment (Red, high; black, average; green low). A positive fold change indicates a higher expression following treatment. A negative fold change indicates a lower expression following treatment. Table 3 lists the fold changes and p values of the differentially expressed miRNAs before and after treatment.

TABLE 3

| miRNA | Fold change | P-value |
|---|---|---|
| hsa-miR-337-3p | −18.20 | 0.01 |
| hsa-miR-605 | −36.08 | 0.02 |
| hsa-miR-597 | −16.23 | 0.02 |
| RNU44.A | −1.80 | 0.03 |
| hsa-miR-650 | 8.01 | 0.04 |

Differentially Expressed miRNAs and CRPS

As presented herein, twenty-three miRNAs were differentially expressed between responders and non-responders before ketamine treatment. Without being bound to a particular theory, an miRNA signature profile is beneficial in predicting treatment outcome.

Further, five miRNAs were differentially expressed in patients who responded to ketamine treatment in samples collected before and after treatment were compared. Target identification and mechanistic studies focusing on these miRNAs can provide insight on 1) the mechanism of action of ketamine on pain patients and 2) the therapeutic utility of miRNA targets.

Inflammatory markers showed that MCP-1, IFNγ and IL-1β levels decreased significantly (p<0.05) following treatment.

### Analgesic Pathway Targets

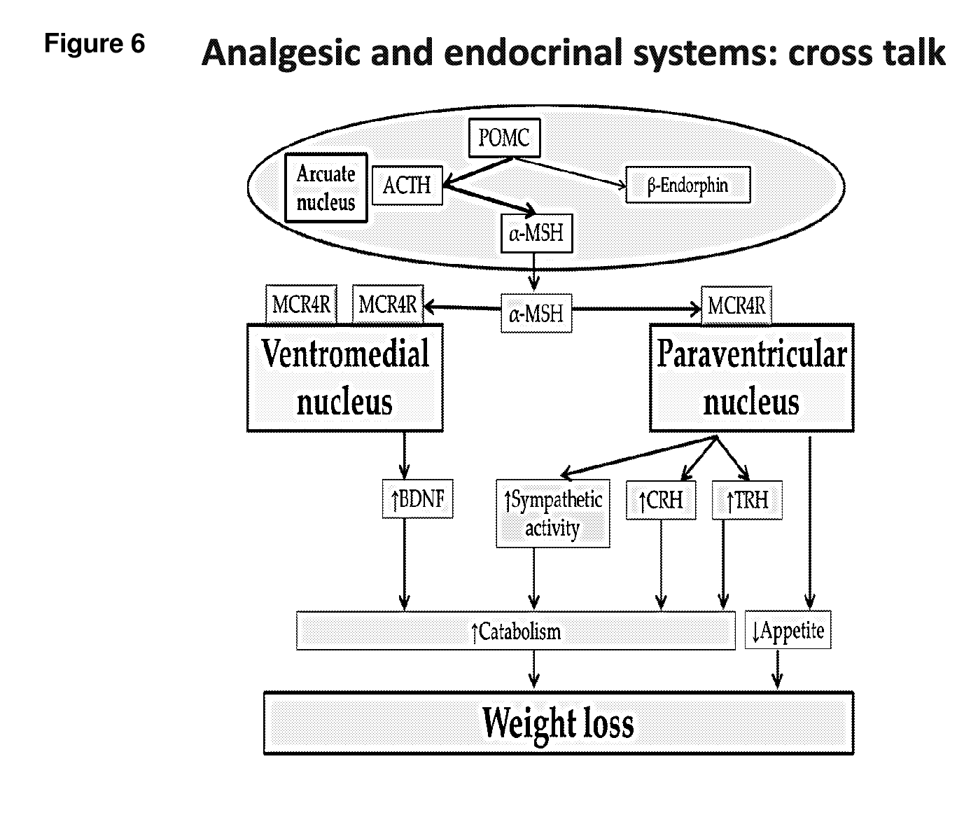

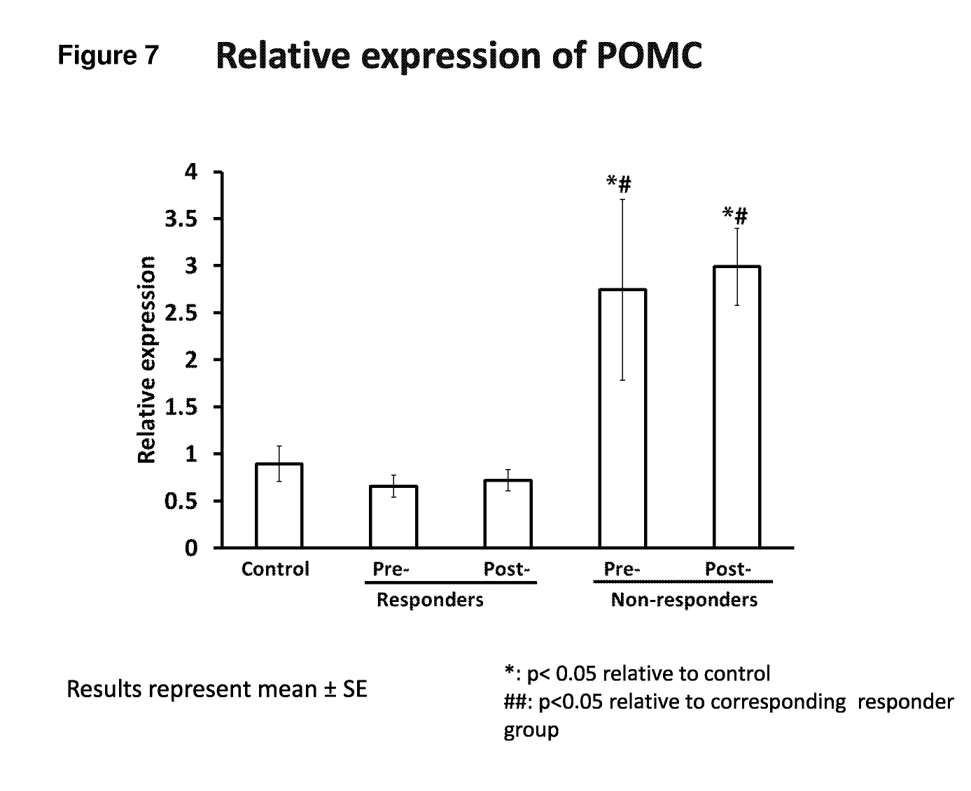

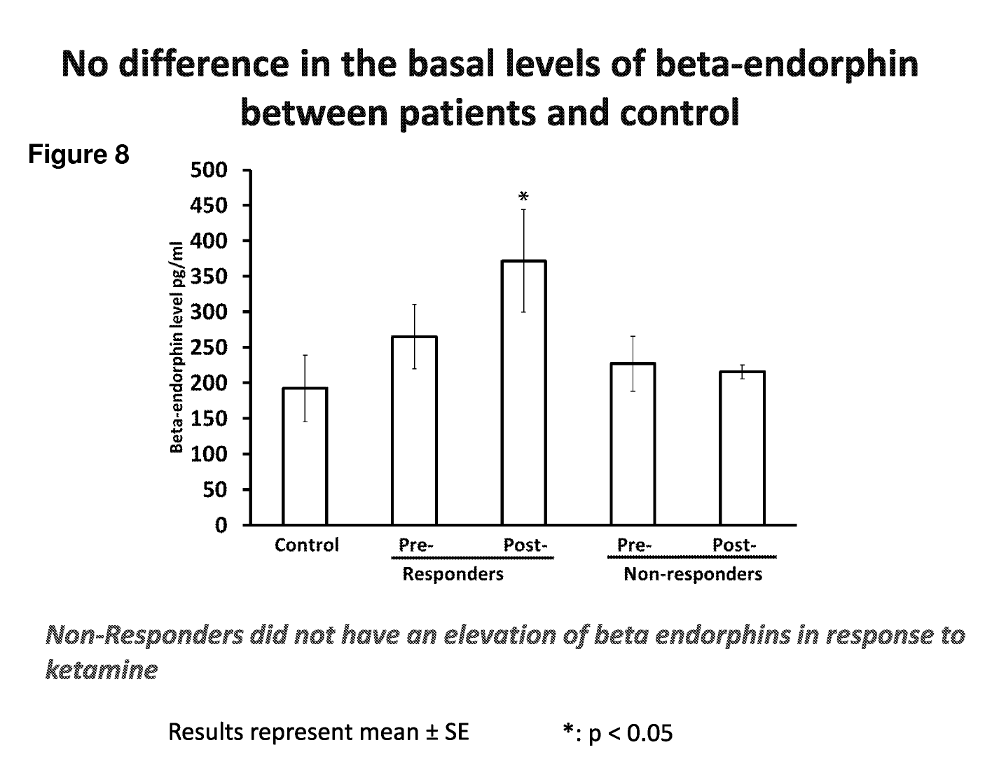

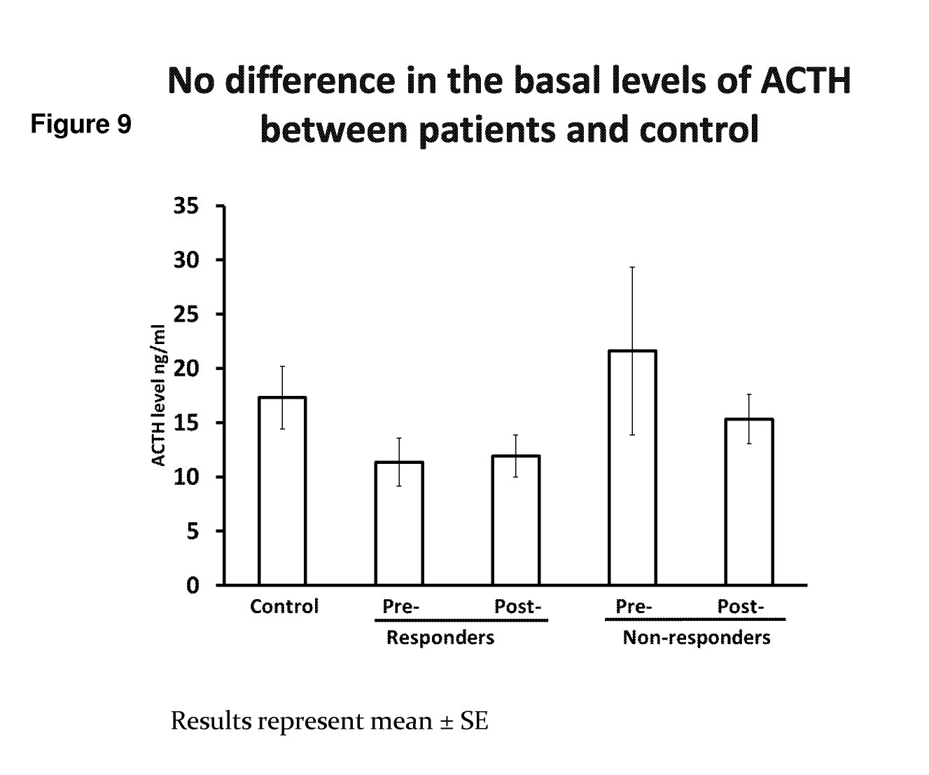

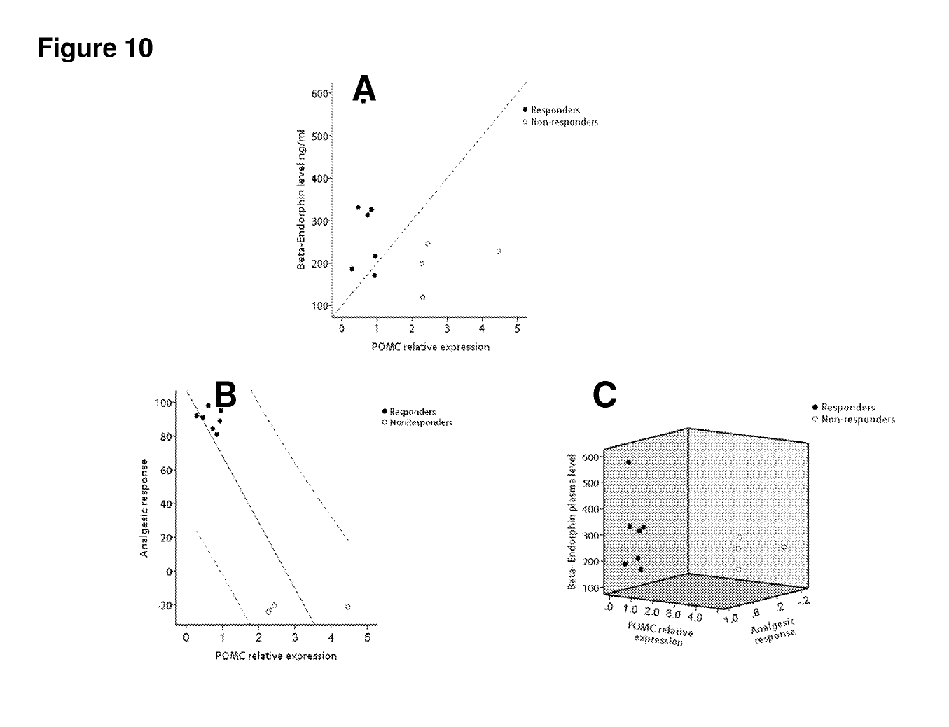

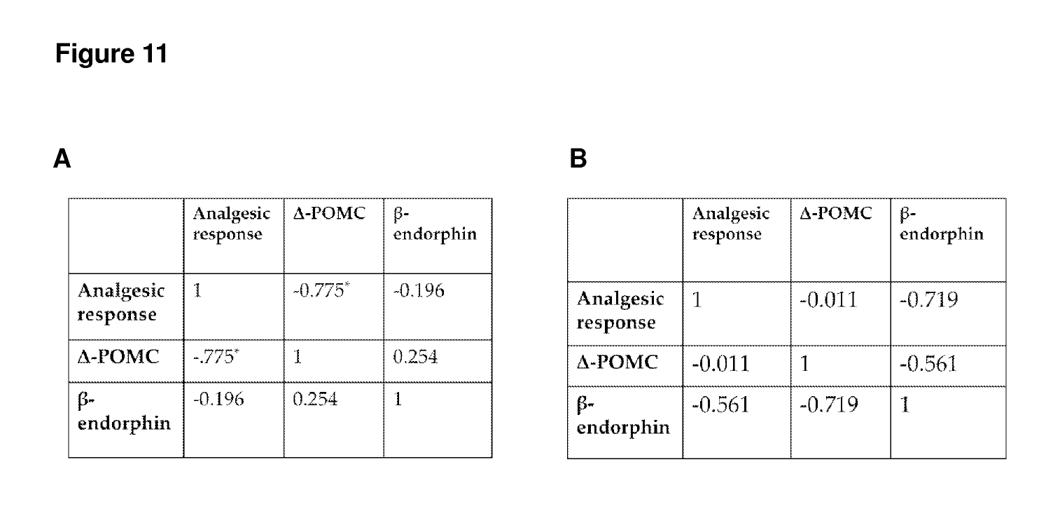

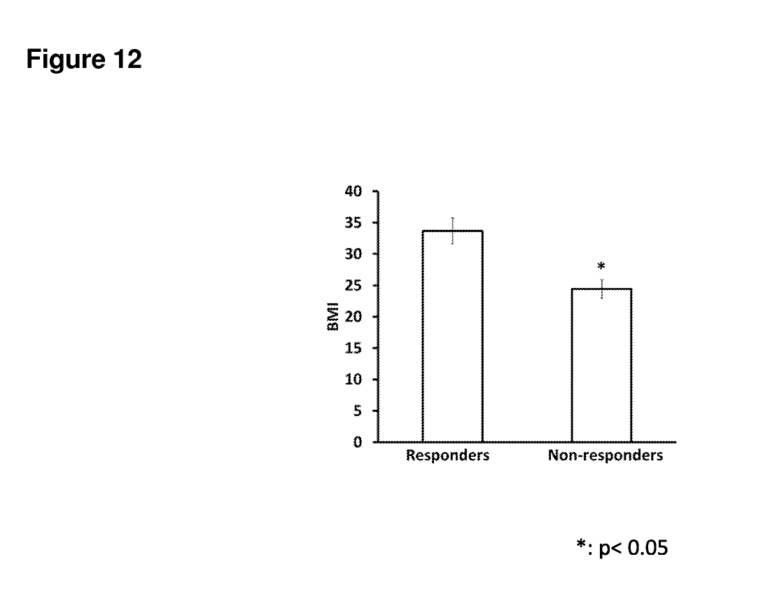

Analgesic pathway targets were selected for validation. Cross talk between analgesic and endocrinal systems is known (FIG. 6). Pro-opiomelanocortin (POMC) is a polypetide precursor of many peptide hormones in the hypothalamopitiutary axis, including Adrenocorticotrophic hormone (ACTH), Melanocyte stimulating hormone (MSH), and Beta-endorphin. Prohormone convertase cleaves POMC yielding important hormonal peptides ACTH, MSH, LSH and β-endorphin. β-endorphin is an important member of the endogenous opioid system. Its release is important in modulation of mood, pain, inflammatory responses. Several studies have linked the expression of POMC and its daughter peptide β-endorphin to placebo effect. Non-Responders to ketamine therapy had increased expression of POMC mRNA in blood relative to the responders and healthy controls (FIG. 7). There was no significant difference in the basal (pre-treatment) plasma levels of β-endorphin between patients and healthy controls. Ketamine therapy induced an increase in β-endorphin in responders but not the non-responders (FIG. 8). No significant differences in the levels of ACTH were observed between patients and control, and responders and non-responders to ketamine therapy (FIG. 9). Correlations between analgesic response, changes in relative expression of POMC (Δ-POMC) and the plasma levels of β-endorphin (Δβ-endorphin) in response to ketamine were plotted (FIG. 10). A negative correlation between the analgesic response and Δ-POMC was observed in responders only (FIG. 11). Non-responders to ketamine therapy had a lower Body Mass Index (BMI) (FIG. 12).

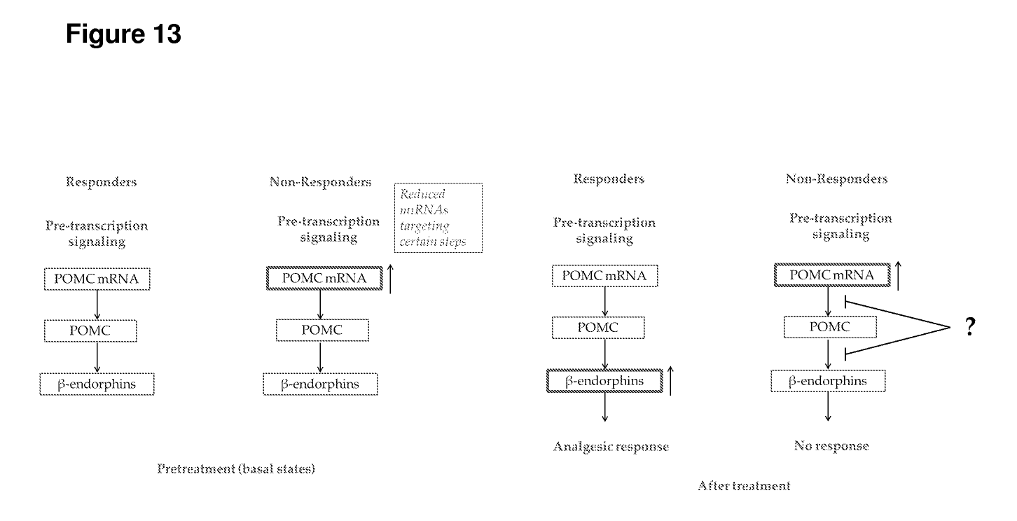

Resistance to the analgesic effect of ketamine is associated with reduced level of β-endorphin in plasma. (FIG. 13). Despite the lower β-endorphin level, the expression of POMC mRNA was high in non-responder patient population. Without being bound to a particular theory, non-responders have an aberration in the POMC-β-endorphin pathway or a relative shift towards ACTH-α-MSH. The enhancement of α-MSH production is expected to target certain nuclei in the hypothalamus with the end result of reduced body weight which was observed in the non-responder population.

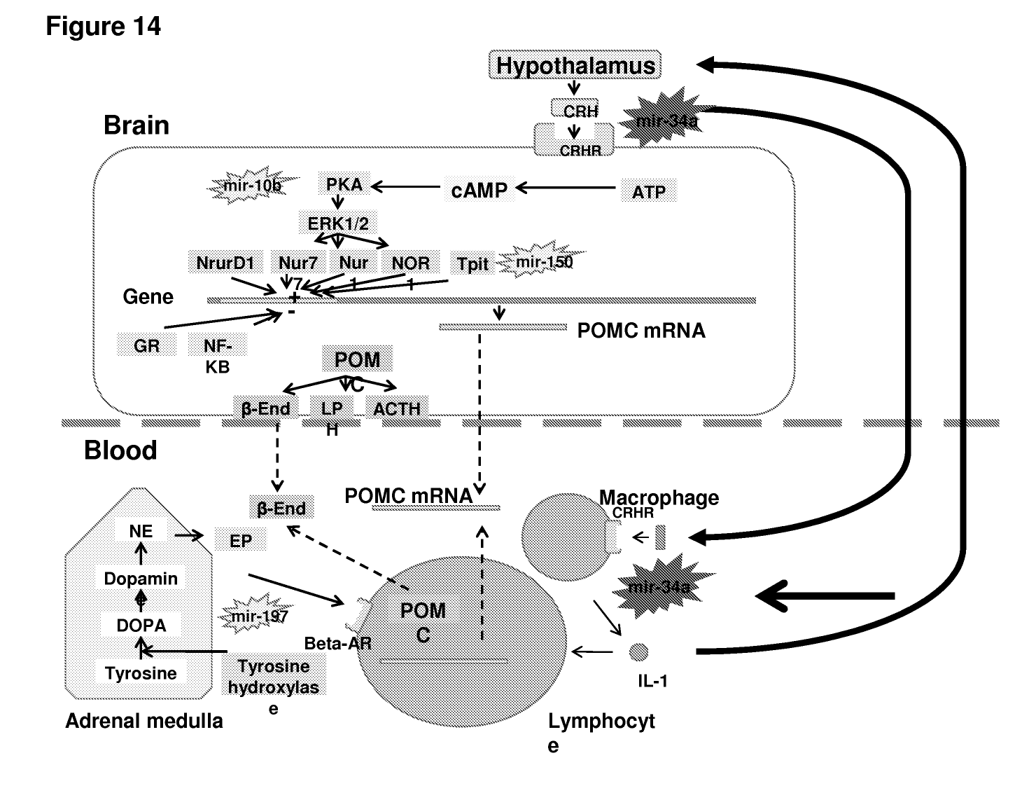

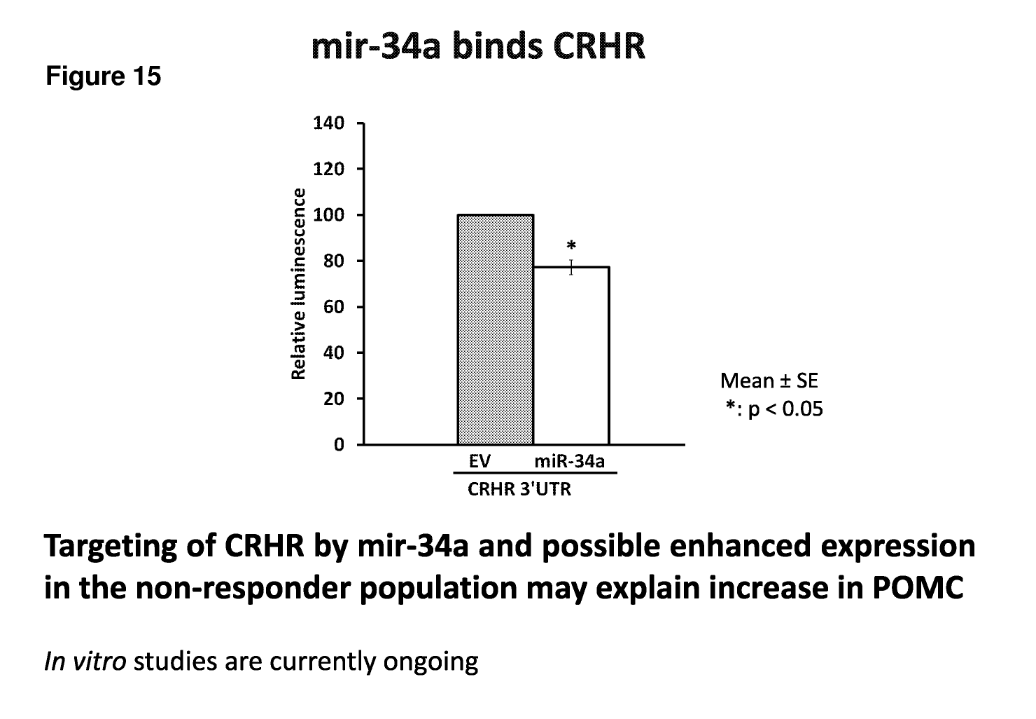

Without being bound to a particular theory, corticotropin-releasing factor receptor (CRHR) targeting by mir-34a has the potential to cause the observed effects (FIG. 14). Using a reporter assay, it was shown that mir-34a binds CRHR (FIG. 15). Thus, targeting of CRHR by mir-34a and enhanced expression in the non-responder population has the potential to explain the increase in POMC. Accordingly, reduced miR-34a in non-responders may cause the upregulation of the CRHR in these patients, which may be associated with dysregulated POMC expression, relatively higher ACTH level, and/or reduced BMI (FIG. 6). Without being bound to a particular theory, differential miRNA expression in CRPS patients and alterations in POMC and its related peptides could render some patients less or unresponsive to ketamine therapy. Theses results indicate that the resistance of CRPS patients to the analgesic effect of ketamine is associated with dysregulation of the endogenous opioid system.

### Targets of Ketamine Pharmacokinetics

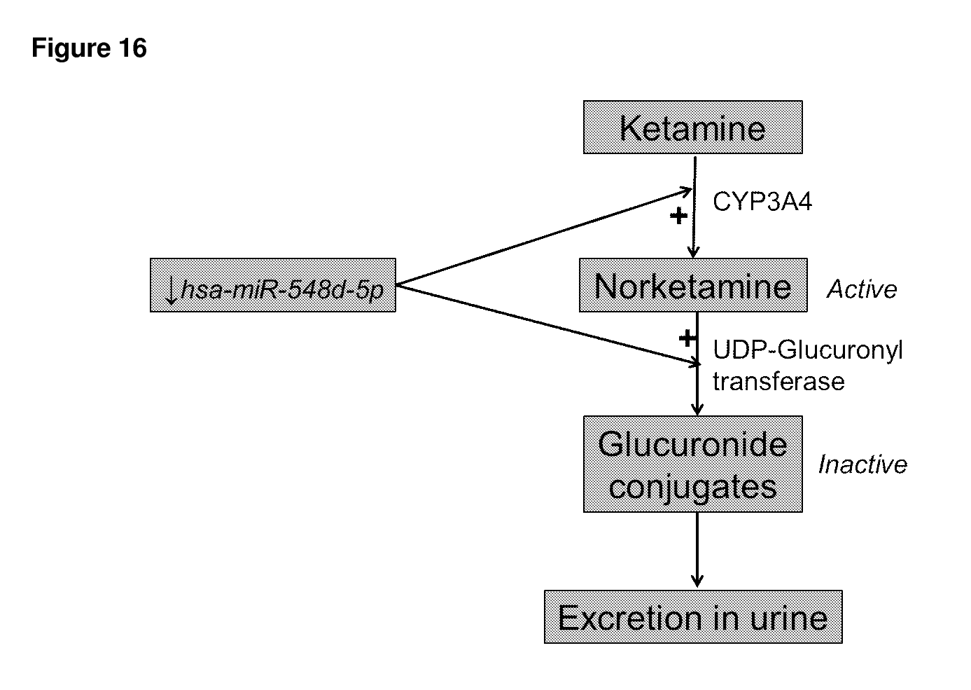

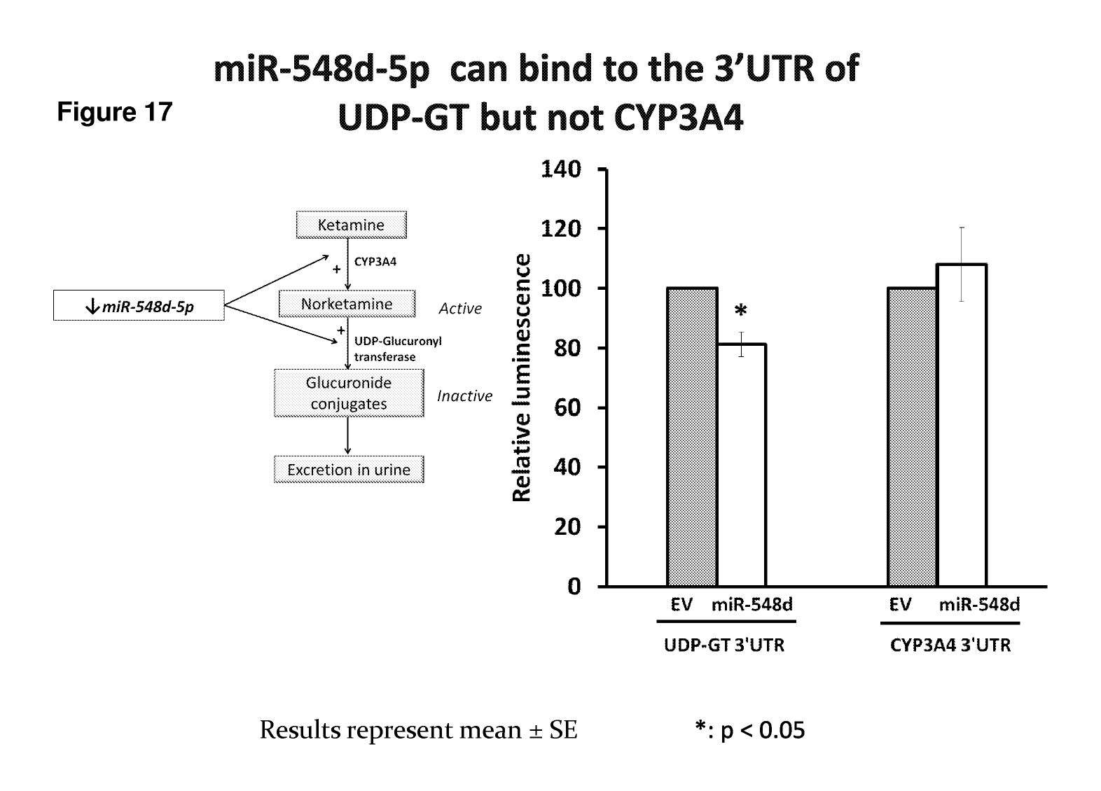

Ketamine resistance may be related to its altered pharmacokinetic profile. Ketamine is known to be demethylated mostly by CYP3A4 followed by conjugation with glucuronic acid to form a readily excretable form. The mRNAs involved in ketamine metabolism are predicted to be targeted by miR548d-5p. This miRNA was reduced in the non-responder population, suggesting enhanced elimination of ketamine in these patients. Without being bound to a particular theory, miR-548d-5p has a potential modulatory role in ketamine metabolism through interactions with CYP3A4 and/or UDP-Glucuronyl transferase (FIG. 16). Using a reporter assay, miR-548d-5p bound the 3′ UTR of UDP-GT but not the 3′ UTR CYP3A4 (FIG. 17).

Differential miRNA expression of miR-34a and/or miR-548d-5p has the potential to explain resistance to ketamine therapy in certain CRPS patients. Without being bound to a particular theory, a decrease in miR-548d-5p is involved in resistance to ketamine therapy through pharmacokinetic modulation, and a reduction in miR-34a contributes to ketamine resistance and alterations in POMC-beta-endorphin pathway. Thus, dysregulation of POMC derived peptides show a link between BMI and treatment response.

### Immune/Inflammation Related Pathway Targets

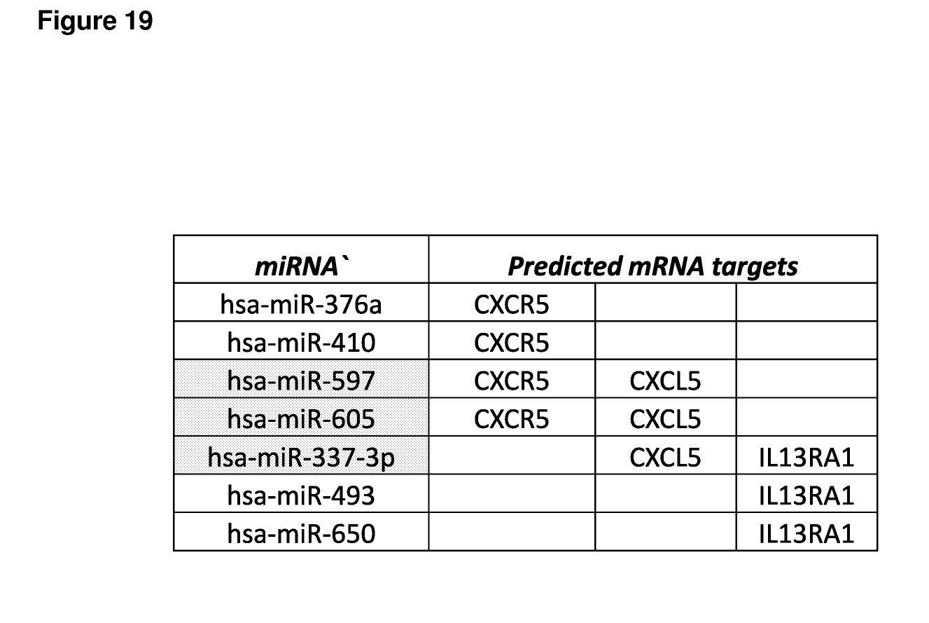

Bioinformatics prediction tools were used to identify potential target genes that can be modulated by miRNAs of interest. Chemokines CXCR5 and CXCL5, which are involved in the immune response, were predicted to be targeted by multiple miRNAs (FIG. 19). CXCL13 is a homeostatic chemokine that regulates B-cell movement. CXCL13 is produced by stromal cells, binds to the CXCR5 receptor and regulates homing of B cells and subsets of T cells to lymphoid follicles Elevated CXCL13 levels were found in the CSF of patients with inflammatory neurological diseases and involved in the formation of ectopic lymphoid tissues within the CNS

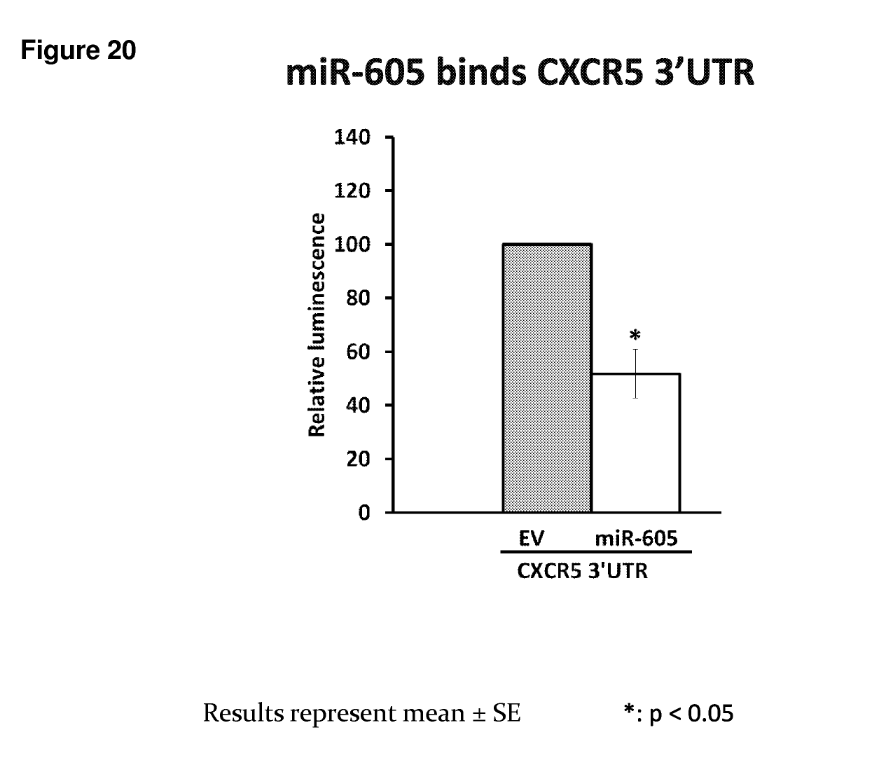

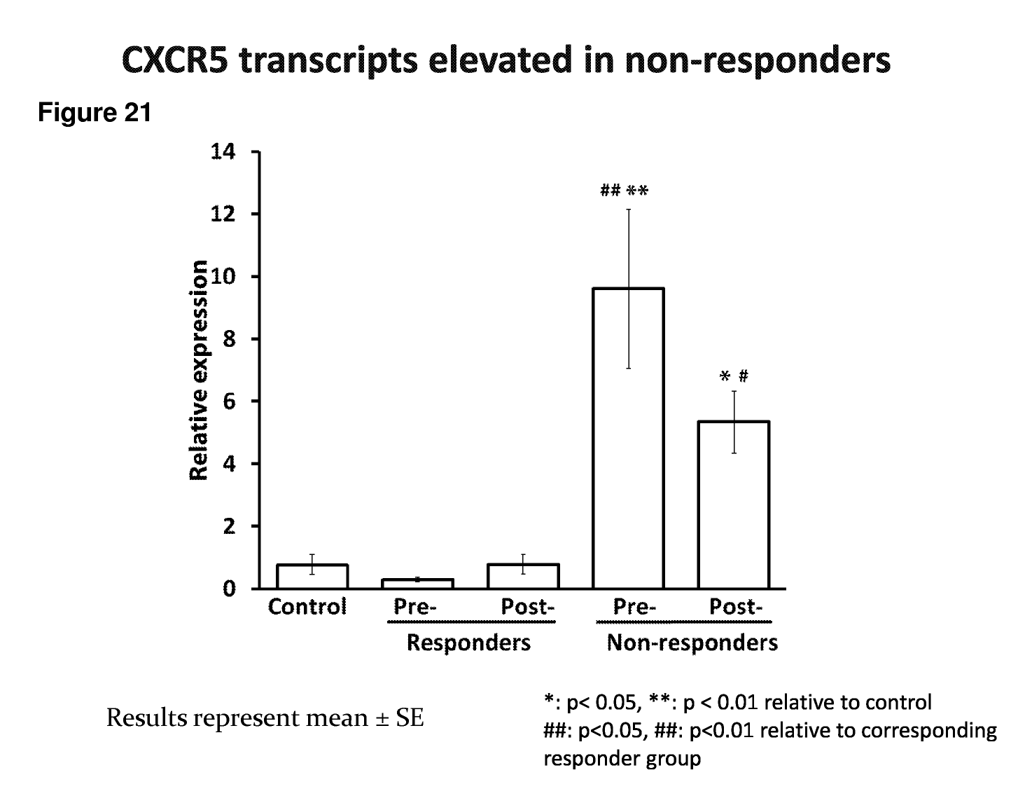

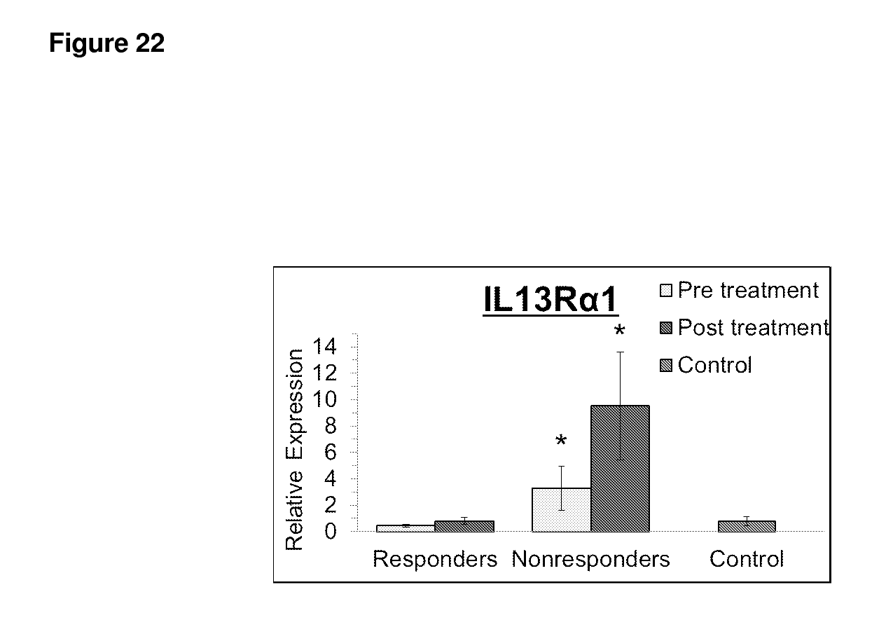

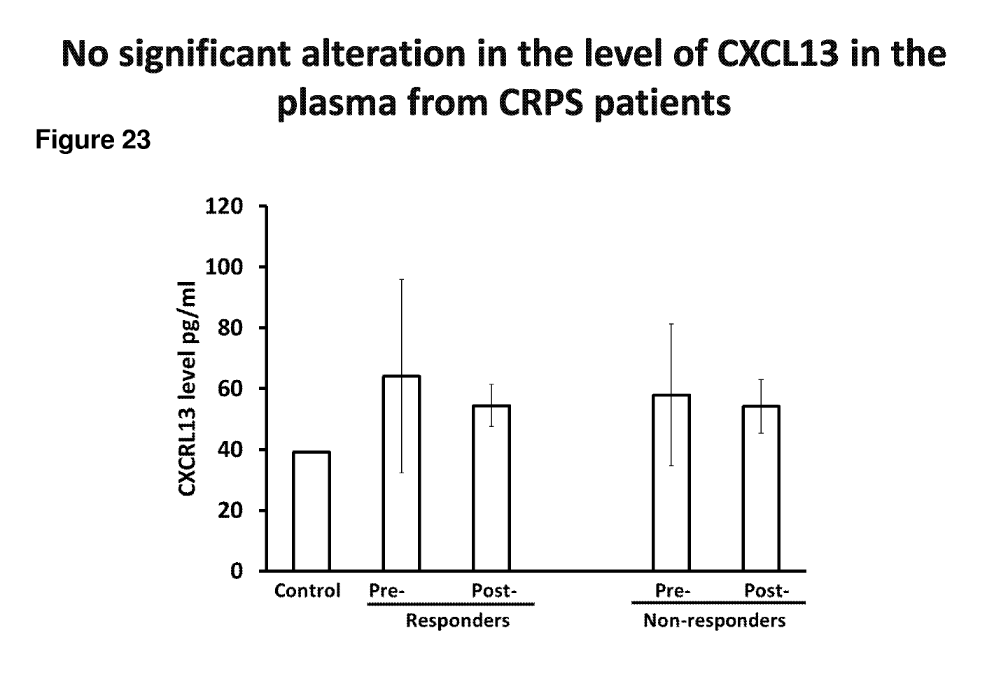

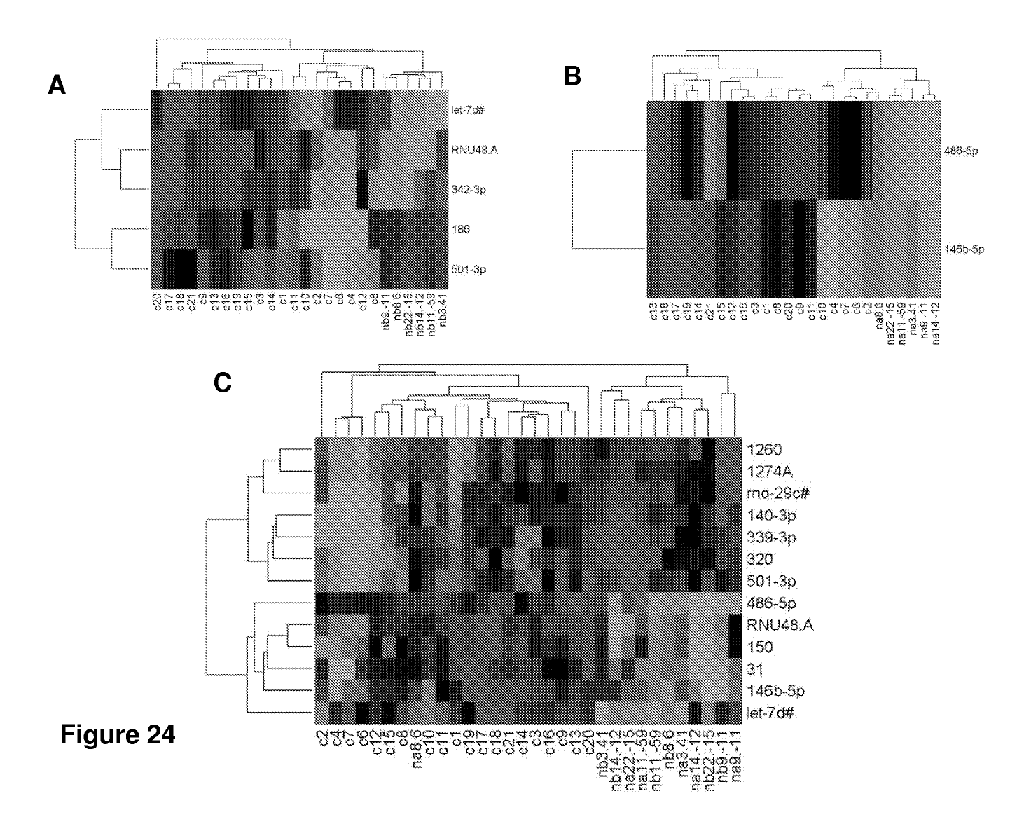

Using a reporter assay, miR-605 bound CXCR5 3′ UTR (FIG. 20). CXCR5 transcripts were elevated in non-responders and that non-responders did not have elevated CXCR5 transcripts (FIG. 21). IL13Rα1 transcripts were observed to be elevated in non-responders (FIG. 22). However, no significant alteration in the level of CXCL13 in the plasma from CRPRS patients (FIG. 23). Clustergrams of miRNA expression in control and non-responders show differentially expressed miRNAs (FIGS. 24A to 24C). Twenty three miRNAs were differentially expressed between responders and non-responders before ketamine treatment. Without being bound to theory, this indicates miRNA signature profiles have the potential to predict treatment outcome. After treatment, five differentially expressed miRNAs were identified in responders. At least three of these miRNAs are predicted to target mRNAs with known roles in inflammation such as CXCR5 and IL13Ra1.

Additional analysis comparing miRNA profiles from responders, non-responders and 20 control subjects enabled identification of additional miRNAs. Some of the downregulated miRNAs in non-responders are validated targets of proinflammatory mediators. Although there were no statistically significant correlations between the reduction of plasma IFNγ, IL-1β and MCP-1 and improvement in pain score in CRPS patients, IFNγ and IL-1β demonstrated a trend towards a positive correlation between reduction in cytokine and pain level.

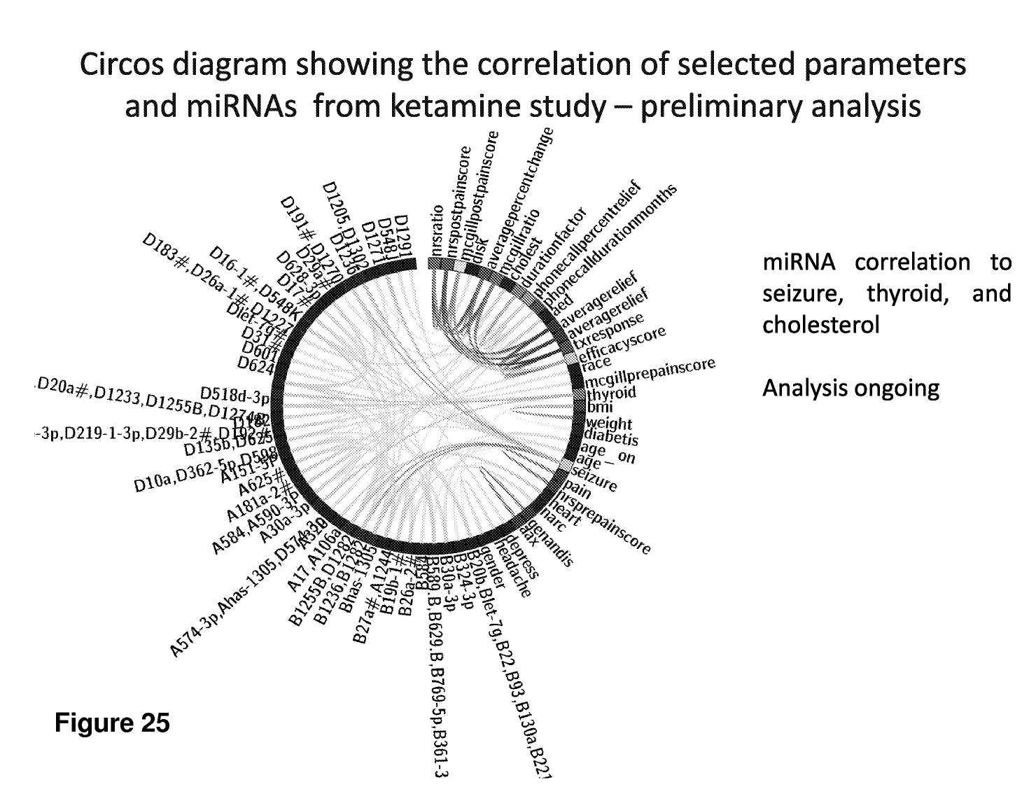

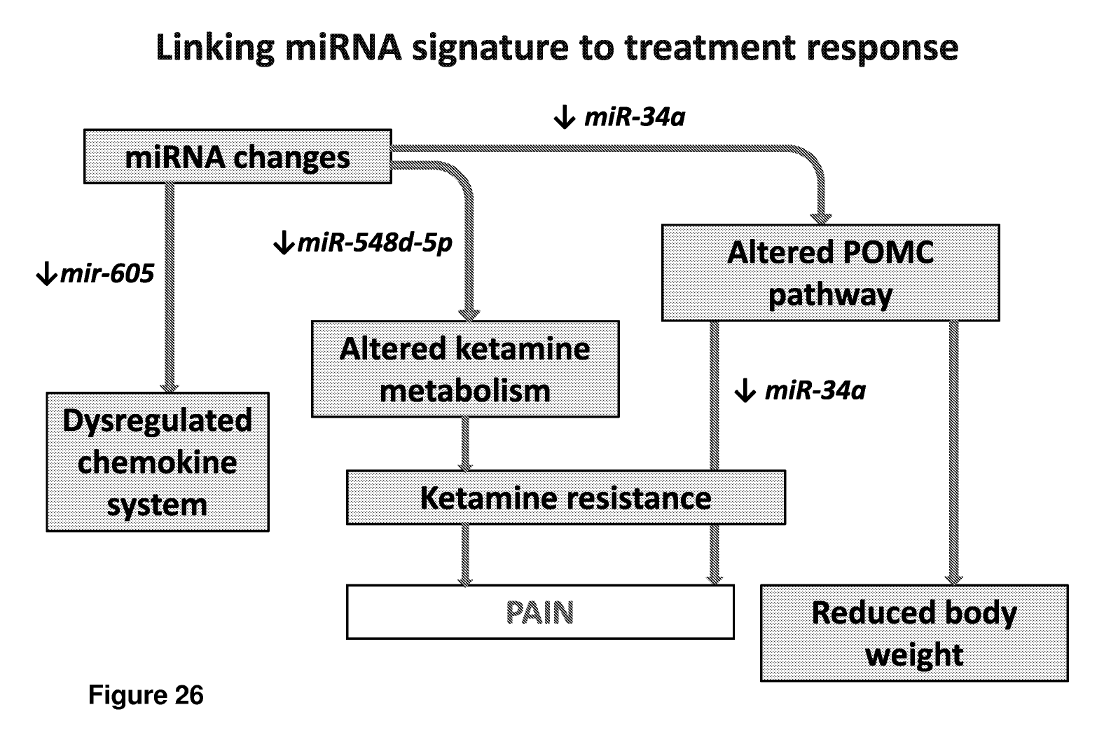

The foregoing studies show correlations of selected parameters and differential expression of miRNAs (FIG. 25). Based on these results, it is proposed that mir-605, miR-548d-5p, and/or miR-34a are involved in the ability of CRPS patients to respond to ketamine treatment. Without being bound to a particular theory, a model linking miRNA signature to treatment response is provided (FIG. 26).

### Example 2

### Exosomes Carry Biomolecular Signatures that Reflect Inflammation-Induced Cellular Alterations and Alleviate Thermal Pain Sensitivity in Mice

In the studies described herein, exosomal miRNAs from CRPS patient serum were profiled and it was determined that miRNAs altered in this chronic pain state are trafficked by exosomes. To determine the global effects of inflammation on exosomal content, RAW 264.7 mouse macrophage-derived exosomes were used to quantify changes in miRNA, mRNA and cytokine levels after stimulation with lipopolysaccharides (LPS). Expression profiling of macrophage-derived exosomal miRNA revealed differential expression of 15 of the 281 detectable miRNAs after LPS stimulation. Several cytokines that mediate inflammation were elevated in exosomes secreted by LPS-stimulated cells. Next-Gen sequencing of exosomal RNA showed alterations in both innate and adaptive immune system pathways. Exosomes from LPS-treated macrophages were sufficient to causes NF-κB activation in vitro and to reduce paw edema after a single intraplantar injection in a mouse model of inflammatory pain. Additionally, macrophage-derived exosomes reduce thermal hyperalgesia 24 hr after induction of inflammatory pain. Overall, the data described herein suggests that macrophage-derived exosomes are immunoprotective, and that exosomal content reflects cellular alterations due to inflammation and pain.

The materials and methods of this Example are now described.

### Cell Culture

RAW 264.7 cells (ATCC) and RAW-Blue cells (Invivogen) were maintained in complete culture media (1×DMEM, 10% heat inactivated FBS). For exosome collection, RAW 264.7 cells (1×10⁷) were plated in 150 mm dishes with complete culture media. At 24 hr, media was replaced with exosome-depleted media (1×DMEM, 10% heat-inactivated FBS depleted of exosomes by ultracentrifugation) with or without 1 μg/ml LPS (Sigma)) and incubated overnight. Media was collected in 50 ml tubes at 24 hr for exosome purification. Human THP1 macrophages were used in some experiments.

### Exosome Purification

Exosome purification from cell culture media was performed as described (McDonald et al., 2013, J Visualized Exp 2013(76):e50294). Centrifugation was used to remove cell debris (500×g for 10 min); the supernatant was transferred and centrifuged (16,500×g for 20 min) Cell-free supernatants were filtered (0.22 μm; VWR, Radnor, Pa.) and exosomes were pelleted by ultracentrifugation (120,000×g for 70 min) The exosomal pellet was resuspended in buffer specific to downstream experiments and vortexed 2×15 seconds. For RNA purification, RNase inhibitors were added after the first centrifugation step at 1 U/ml (RNAsin Plus; Promega, Madison, Wis.) and at all subsequent steps at 1 U/μl. For purification from human samples, serum was diluted 1:1 with 1x PBS (−) Mg2+ and Ca2+(Corning 21-031-CV; Corning, N.Y.) and spun at 2000×g for 30 min at 4° C. The sample was transferred to a centrifuge tube and spun at 12,000×g for 45 min at 4° C., then transferred to an ultracentrifuge tube and spun at 110,000×g for 2 hr at 4° C. The pellet was resuspended in 1×PBS minus Mg²⁺ and Ca²⁺ and spun for an additional hour at 110,000×g before resuspension in RNA lysis buffer.

### Human Serum-Derived Exosomes

Patients with CRPS were recruited from the neurology pain clinic at Drexel University College of Medicine and met the clinical Budapest criteria for CRPS Harden et al., 2007, Pain Med 8(4):326-331. Healthy painfree control subjects were recruited from the community's general population. Blood samples were drawn from the cubital vein of subjects at rest, collected in serum-separating tubes and spun at 1940×g for 15 min at 4° C. after 30 min incubation at room temperature.

### Preparation of Exosomes for EM

Droplets of purified exosomes resuspended in 1% glutaraldehyde in 0.1 M sodium phosphate buffer were placed on 300-mesh carbon-coated polyvinyl formal copper grids (Formvar, Electron Microscopy Sciences Hatfield, Pa.) and left to adsorb for 30 min After excess buffer was removed, dry grids were washed with deionized water and stained with 1% aqueous uranyl acetate before TEM analysis. For immunolabeling, exosomes were resuspended in 2% paraformaldehyde and droplets were left to adsorb on 300-mesh carbon-coated Formvar nickel grids for 20 min After 2 washes in 1×PBS and 4 washes in 1x PBS/50 mM glycine, grids were incubated with blocking buffer (5% BSA/0.05% polysorbate 20/5% FBS in 1×PBS) for 10 min. The grids were immunolabeled with mouse anti-CD81 (1:100, Sigma) in 1:5 dilution of blocking buffer in 1×PBS for 30 min at room temperature. The unbound antibody was removed with 6 washes in 1:10 dilution of blocking buffer and then grids were incubated with 10 nm gold-labeled anti-rabbit IgG (1:25, Sigma) for 20 min at room temperature. After the unbound antibody was removed with 6 washes in 1:10 dilution of blocking buffer, grids were incubated in 1% glutaraldehyde for 5 min, washed with water, and stained with uranyl acetate as above.

### Western Blotting and Cytokine Array

Exosomes were resuspended in radioimmunoprecipitation assay buffer (Thermo Scientific, Waltham, Mass.) containing Halt protease inhibitor cocktail (Thermo Scientific) and the protein concentration was determined by Bradford analysis. For western blotting, the lysate was run on a 12% SDS-PAGE (NuPAGE, Novex/Life Technologies) for 1.5 hr at 150 V. After 1 hr transfer at 100 V, the nitrocellulose membrane was blocked with 5% nonfat dry milk in Tris-buffered saline and polysorbate 20 for 1 hr, incubated with rabbit anti-HSP70 (Abcam, Cambridge, UK) or rabbit anti-TSG101 (Genetex, Irvine, Calif.) overnight and then with goat anti-rabbit IgG-HRP (System Biosciences, Mountain View, Calif.). LPS was detected after 1 hr incubation with mouse anti-LPS (Abcam, ab35654) and goat antimouse IgG-HRP (Abcam, ab6789). Proteins were detected by Immobilon (Thermo Scientific) detection reagent and film exposure. For cytokine array, 100 μg of protein was incubated with the blots according to manufacturer's specifications (R&D Systems, Minneapolis, Minn.).

RNA Sequencing and miRNA Profiling

The SOLiD whole transcriptome analysis kit protocol with the fragmentation step omitted was used to generate a cDNA library for each sample. Total RNA, ranging in size from kilobases down to 10-mers, was purified from exosomes using the mirVana miRNA isolation kit (Life Technologies) following manufacturer's protocol. RNA concentration was measured using Nanodrop 1000 (NanoDrop Technologies, Wilmington, Del.). Total RNA from 3 independent exosome purifications was pooled to obtain 4 μg exosomal RNA per library (due to a limited amount of RNA in individual preparations), analyzed for integrity using the Agilent RNA 6000 Pico Kit (RNA integrity number between 1.6 and 2.1), and gel purified. Sequencing adapter ligation and cDNA reverse transcription were performed with SOLiD Total RNA-seq kit. DNA fragments in the target range of 150 to 500 bp were enriched using Agencourt AMPure XP PCR bead capture purification (Beckman Coulter; Brea, Calif.) before sequencing 50-bp pieces with no paired ends. The SOLiD 5500XL high-throughput sequencing platform (Applied Biosystems, Carlsbad, Calif.) was used for sequencing.

Sequencing reads were aligned to the mouse reference genome version mm9 (July, 2007) and transcripts were assembled based on refGene annotations (dated Dec. 16th, 2012) obtained from the UCSC Genome Browser (Karolchik et al., 2014, Nucleic acids research 42(1):D764-770) and non-coding RNA transcript definitions (dated Dec. 19th, 2012) from the fRNAdb database (Kin et al., 2007, Nucleic acids research 35(Database issue):D145-148) at ncrna.org. Reads were mapped using the LifeScope Whole Transcriptome Pipeline with default parameters, which effectively maps RNA fragments down to 22 nucleotides in length. The Cufflinks algorithm (Trapnell et al., 2013, Nature biotechnology 31(1):46-53) was used for transcript assembly, abundance estimation and differential expression analysis, using the reference transcript annotation as a guide. Results generated from Cufflinks were investigated using the CummeRbund package (www.R-project.org). Following differential expression analysis, transcripts were annotated using information from the Molecular Signatures Database (Mathivanan et al., 2012, Nucleic acids research 2012; 40(1):D1241-1244; Subramanian et al., 2005, Proc Natl Acad Sci USA 102(43):15545-15550) for biological interpretation.

RNA Sequencing and miRNA Profiling

TLDA microfluidic cards (Life Technologies) were used for miRNA profiling as previously described. Thirty nanograms of total RNA were used for each cDNA synthesis reaction. Taqman preamplification reaction was performed before the samples were loaded into the TLDA cards as described previously (Fevrier et al., 2004, Proc Natl Acad Sci USA 101(26):9683-9688). For miRNAs profiled from exosomes collected from RAW 264.7 cells, significance was determined by applying a P value cutoff of 0.05 to the results of a paired-samples t test. For human exosomal miRNAs, significance was determined by applying the Benjamini-Hochberg false discovery rate correction to the results of a 2-tailed t test.

qPCR Validation of Exosomal mRNAs

cDNA was synthesized from 5 ng purified exosomal RNA using the WT-Ovation RNA Amplification System from NuGEN (San Carlos, Calif.). was used Taqman assays were performed in a reaction volume of 20 μl and the components used were 10 μl Taqman Fast Universal PCR master mix (2×) no AmpErase UNG, 1 μl Taqman gene expression assay mix (20×), 2 μl cDNA (100 ng), and 7 μl RNase-free water. Gapdh was used as the normalizer and a t test was used to perform statistical analysis. Assay IDs: Mm00443111_m1 [CCL4], Mm00436450_m1 [CXCL2], Mm00441242_m1 [CCL2], Mm00443260_g1 [TNF]) Mm00501607_m1 [Creb1] and Mm00497193_m1 [Zeb2] (Applied Biosystems).

### NF-κB Reporter Assay

RAW-Blue cells (InvivoGen, San Diego, Calif.), maintained in complete media (1×DMEM, 10% heat-inactivated FBS), were seeded into a 96-well plate in exosome-free media on the day of the assay. Exosomes purified from RAW 264.7 cells without or after LPS stimulation were added at 4 concentrations. After 24 hr, QUANTI-Blue assay was performed with QUANTI-Blue media, prepared as described by the manufacturer (InvivoGen). To 150 μl QUANTI-Blue media, 50 μl conditioned media was added and incubated at 37° C. for 1 h. Plates were read at 650 nm (Spectramax Plus, Molecular Devices, Sunnyvale, Calif.).

### CFA-Induced Inflammatory Pain Model

All behavioral tests were performed using 8-week-old C57BL/6 male mice purchased from Taconic (Cranbury, N.J.). Mice were housed in 12-h light/dark cycles. Behavioral assays were performed by researchers blinded to the treatment received. The CFA-induced inflammatory pain model was established and CFA-induced mechanical and thermal hypersensitivity was measured as described (Pan et al., 2012, J Pharmacology 343:661-672). Baseline measurements were obtained before initiation of treatment. Twenty microliters of 50% CFA was administered by intraplantar injection into the right hind paw. Mechanical sensitivity was measured using a series of von Frey filaments (North Coast Medical, Inc., San Jose, Calif.). The smallest monofilament that evoked paw withdrawal responses on 3 of 5 trials was taken as the mechanical threshold. Thermal sensitivity was measured using the Hargreaves method. The baseline latencies were set to approximately 10 seconds with a maximum of 20 seconds as the cutoff to prevent potential injury. The latencies were averaged over 3 trials separated by 15-min intervals. At 3 hr post-CFA injection and after confirming that the animals were sensitive, 20 μl exosomes (0.5 μg) in PBS were injected intraplanar to the right hind paw. Paw thickness was recorded (3 hr and 1, 2, and 5 day) and paw withdrawal was measured by the von Frey (1, 5, 10, 15, and 21 days) and Hargreaves methods (3 hr and 1, 5, and 10 days) (n=9).

### Data Analysis

Data are presented as mean±SEM. Treatment effects were statistically analyzed with a 1- or 2-way ANOVA. Pairwise comparisons between means were tested using the post hoc Bonferroni method. Error probabilities of P<0.05 were considered statistically significant.

The results of this Example are now described.

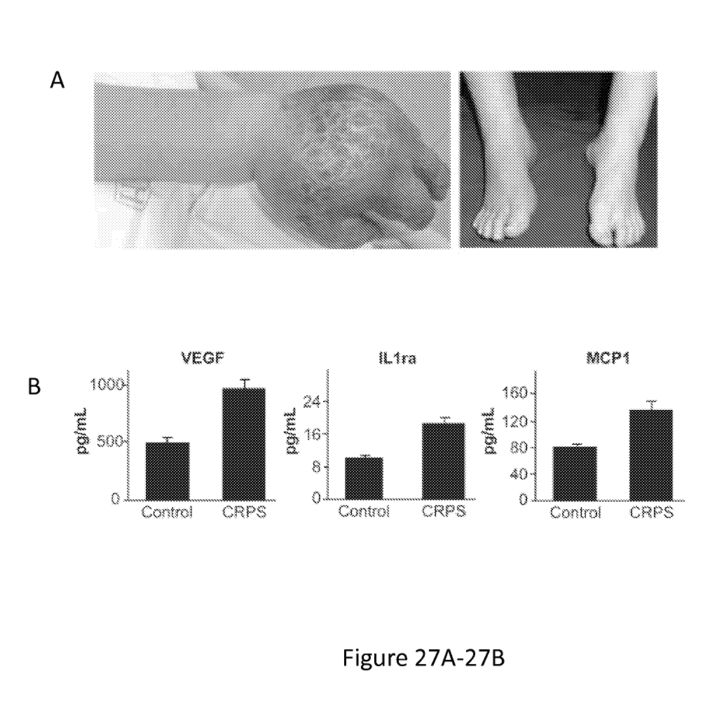

MicroRNAs (miRNAs) are small noncoding RNAs that bind mRNA targets via a complementary seed sequence and repress translation. miRNAs circulate in bodily fluids such as blood and can be used as biomarkers in various diseases. A previous study analyzing miRNA levels and inflammatory markers in the blood of patients with CRPS showed an increase in inflammatory markers and differential expression of 18 miRNAs circulating in the blood (Orlova et al., 2011, J Transl Med 9:195). The objectives of the studies described herein included characterizing alterations in miRNA, mRNA, and cytokines in exosomes secreted by RAW 264.7 murine macrophage cells in response to inflammatory stimulus, determining the effect of inflammatory stimuli on exosome-mediated intercellular communication in vitro and in vivo, and determining if miRNA alterations seen in CRPS patients are reflected in the exosomal fraction of blood. FIG. 27A depicts some of the signs of CRPS, which include sensory (pain and hyperalgesia), autonomic (alterations in skin temperature, color, increased sweating) and motor (tremor, dystonia) disturbances. FIG. 27B depicts the results of experiments showing that the inflammatory markers VEGF, IL1Ra, and MCP1 were significantly increased in CRPS patients vs. control samples, with p values 0.0002, 0.0004, and 0.0005, respectively. The levels of IL-4, IL-5, IL-6, 11-8, and TNFα also showed an increase that did not reach statistical significance in this study. FIG. 27C is a schematic representation of exosome formation. Exosomes arise from cytosolic multivesicular bodies (MVBs) which fuse with the plasma membrane to release exosomes (Ludwig and Giebel, 2011, The International J Biochemistry & Cell Biol 44:11-5).

### Exosome Characterization

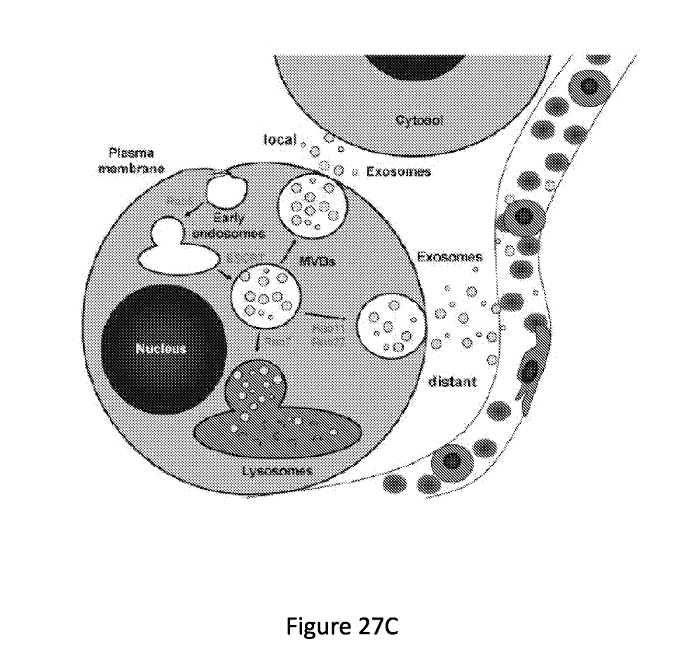

Experiments were conducted to permit the morphological and biochemical characterization of exosomes. Exosomes were purified from RAW 264.7 cell culture media and human serum. After purification, transmission electron microscopy (TEM) was used in conjunction with immune-gold labeling to analyze the specificity and morphology of exosomes purified from naive and LPS-stimulated RAW 264.7 cell culture media. Exosomes maintain a vesicular morphology with an approximate diameter of 100 nm and show immunoreactivity for CD81, a tetraspannin protein found in exosomal membranes (FIGS. 28A and 28B). Specificity of exosome preparations from naive or LPS-stimulated RAW 264.7 cells was additionally verified by western blotting for the presence of HSP70, TSG101 (tumor susceptibility gene), and LPS. All exosomal protein lysates showed specificity for HSP70 and TSG101 (FIG. 28C). LPS was undetectable in exosomes after LPS stimulation for 24 hr. The integrity of total exosomal RNA was analyzed using the Agilent Bioanalyzer (Agilent Technologies, Santa Clara, Calif.) (FIGS. 28D and 28E). The concentrations of exosomal RNA from macrophage cell culture media without and with LPS stimulation were 2.4±0.3 and 2.0±0.5 ng/ml, respectively. Consistent with previous reports, exosomal RNA is relatively low in concentration and does not contain a prominent 18S or 28S rRNA peak (Crescitelli et al., 2013, Journal of extracellular vesicles 2013; 2).

### LPS Stimulation Alters Exosomal RNA Populations

Exosomes contain a variety of coding and noncoding RNAs, but a comprehensive analysis of the total RNA population before and after an inflammatory stimulus has not been undertaken. Quantitative PCR (qPCR) was performed on exosomal miRNA before and after LPS stimulation using Taqman low-density array (TLDA) cards to detect and quantitate up to 758 miRNAs. The assays detected 433 miRNAs in exosomes derived from naive and LPS-stimulated RAW 264.7 cells (Table 4).

TABLE 4
miRNAs in macrophage-derived exosomes
Purified exosomal RNA samples from RAW 264.7 cells (untreated
and stimulated with LPS) were profiled for 758 miRNAs using
TLDA cards. The ΔΔCt and P values of detected miRNAs for
4 separate treatments including the number of times each miRNA
was detected are shown. Homologues of miRNAs identified in our
previous study to be differentially expressed in whole blood
from patients with CRPS shown in bold.

| | minus | plus | | | fold |
|---|---|---|---|---|---|
| miRNA | LPS | LPS | ddct | p-value | change |
| mmu-let-7b | 4 | 4 | 1.704 | 0.037 | −3.258 |
| mmu-miR-320 | 4 | 3 | 3.038 | 0.128 | −8.215 |
| mmu-miR-532-3p | 3 | 3 | −2.170 | 0.100 | 4.501 |
| mmu-let-7a | 1 | 2 | −2.311 | 0.218 | 4.961 |
| mmu-miR-720 | 4 | 4 | 0.482 | 0.072 | −1.397 |
| rno-miR-664 | 4 | 4 | −0.203 | 0.797 | 1.151 |
| mmu-miR-1939 | 3 | 2 | 0.774 | 0.466 | −1.710 |
| mmu-let-7c | 3 | 4 | −0.519 | 0.759 | 1.433 |
| mmu-let-7d | 3 | 3 | −2.851 | 0.497 | 7.213 |
| mmu-let-7e | 3 | 4 | 1.530 | 0.517 | −2.889 |
| mmu-let-7g | 4 | 3 | 1.000 | 0.481 | −2.000 |
| mmu-let-7i | 4 | 4 | 0.115 | 0.912 | −1.083 |
| mmu-miR-1 | 1 | 1 | n/a | n/a | n/a |
| rno-miR-1 | 1 | 0 | n/a | n/a | n/a |
| mmu-miR-7a | 3 | 2 | 0.573 | 0.693 | −1.487 |
| mmu-miR-7b | 1 | 2 | −2.147 | 0.166 | 4.428 |
| MammU6 | 4 | 4 | 1.350 | 0.104 | −2.549 |
| mmu-miR-9 | 1 | 2 | −1.630 | 0.206 | 3.096 |
| mmu-miR-10a | 2 | 1 | n/a | n/a | n/a |
| mmu-miR-10b | 0 | 1 | n/a | n/a | n/a |
| mmu-miR-15a | 2 | 2 | −1.221 | 0.196 | 2.331 |
| mmu-miR-15b | 3 | 4 | −0.658 | 0.452 | 1.578 |
| mmu-miR-16 | 4 | 4 | 0.493 | 0.775 | −1.408 |
| mmu-miR-18a | 3 | 2 | 0.371 | 0.123 | −1.293 |
| mmu-miR-19a | 3 | 4 | −1.339 | 0.548 | 2.530 |
| mmu-miR-19b | 4 | 4 | −0.082 | 0.821 | 1.058 |
| mmu-miR-20a | 4 | 3 | 1.715 | 0.502 | −3.282 |
| mmu-miR-20b | 4 | 4 | 1.331 | 0.260 | −2.516 |
| mmu-miR-21 | 3 | 4 | −2.794 | 0.129 | 6.934 |
| mmu-miR-23b | 2 | 1 | 1.247 | 0.613 | −2.373 |
| mmu-miR-24 | 4 | 4 | 0.395 | 0.049 | −1.315 |
| mmu-miR-25 | 3 | 2 | 0.960 | 0.613 | −1.945 |
| mmu-miR-26a | 4 | 3 | 0.249 | 0.922 | −1.188 |
| mmu-miR-26b | 3 | 2 | 0.789 | 0.654 | −1.728 |
| mmu-miR-27a | 3 | 3 | 0.899 | 0.056 | −1.864 |
| mmu-miR-27b | 3 | 2 | 1.699 | 0.392 | −3.246 |
| mmu-miR-28 | 2 | 2 | −0.856 | 0.437 | 1.811 |
| mmu-miR-29b | 1 | 1 | n/a | n/a | n/a |
| mmu-miR-29c | 2 | 2 | −1.415 | 0.231 | 2.666 |
| mmu-miR-30b | 4 | 4 | −0.087 | 0.758 | 1.062 |
| mmu-miR-30c | 4 | 4 | 0.288 | 0.721 | −1.221 |
| mmu-miR-30d | 4 | 2 | 2.688 | 0.214 | −6.445 |
| mmu-miR-34a | 1 | 1 | −0.820 | 0.383 | 1.765 |
| mmu-miR-93 | 3 | 4 | −0.004 | 0.997 | 1.003 |
| mmu-miR-98 | 0 | 1 | n/a | n/a | n/a |
| mmu-miR-99a | 2 | 2 | 3.246 | 0.506 | −9.485 |
| mmu-miR-99b | 3 | 3 | −1.015 | 0.319 | 2.021 |
| mmu-miR-100 | 1 | 1 | n/a | n/a | n/a |
| mmu-miR-101a | 3 | 2 | 0.639 | 0.436 | −1.558 |
| mmu-miR-103 | 2 | 2 | 0.367 | 0.796 | −1.289 |
| mmu-miR-105 | 1 | 0 | n/a | n/a | n/a |
| mmu-miR-107 | 1 | 0 | n/a | n/a | n/a |
| mmu-miR-122 | 1 | 2 | −2.045 | 0.376 | 4.127 |
| mmu-miR-124 | 1 | 1 | n/a | n/a | n/a |
| mmu-miR-125a-3p | 3 | 4 | −1.585 | 0.431 | 2.999 |
| mmu-miR-125a-5p | 3 | 4 | −2.967 | 0.076 | 7.819 |
| mmu-miR-125b-5p | 3 | 4 | −2.551 | 0.194 | 5.862 |
| mmu-miR-126-3p | 4 | 3 | 1.600 | 0.535 | −3.032 |
| mmu-miR-126-5p | 1 | 3 | −2.550 | 0.008 | 5.858 |
| mmu-miR-127 | 1 | 1 | n/a | n/a | n/a |
| mmu-miR-128a | 1 | 3 | −3.759 | 0.154 | 13.538 |
| mmu-miR-129-3p | 0 | 1 | n/a | n/a | n/a |
| mmu-miR-130a | 1 | 1 | n/a | n/a | n/a |
| mmu-miR-130b | 3 | 3 | 0.402 | 0.176 | −1.321 |
| mmu-miR-132 | 2 | 2 | −2.399 | 0.131 | 5.273 |
| mmu-miR-133a | 1 | 2 | −1.197 | 0.544 | 2.293 |
| mmu-miR-133b | 2 | 1 | n/a | n/a | n/a |
| mmu-miR-135b | 1 | 2 | −2.211 | 0.438 | 4.630 |
| mmu-miR-137 | 0 | 1 | n/a | n/a | n/a |
| mmu-miR-138 | 0 | 1 | n/a | n/a | n/a |
| mmu-miR-139-3p | 4 | 3 | 2.950 | 0.215 | −7.726 |
| mmu-miR-139-5p | 4 | 4 | −0.145 | 0.804 | 1.106 |
| mmu-miR-140 | 3 | 4 | −2.144 | 0.375 | 4.419 |
| mmu-miR-141 | 0 | 1 | n/a | n/a | n/a |
| mmu-miR-142-3p | 4 | 4 | −0.526 | 0.582 | 1.440 |
| mmu-miR-142-5p | 2 | 1 | n/a | n/a | n/a |
| snoRNA135 | 1 | 0 | n/a | n/a | n/a |
| mmu-miR-145 | 3 | 2 | 1.390 | 0.215 | −2.621 |
| mmu-miR-146a | 4 | 4 | −3.295 | 0.019 | 9.814 |
| mmu-miR-146b | 2 | 2 | −1.428 | 0.353 | 2.690 |
| mmu-miR-148a | 0 | 1 | n/a | n/a | n/a |
| mmu-miR-148b | 1 | 2 | −1.459 | 0.297 | 2.749 |
| mmu-miR-150 | 4 | 4 | −0.833 | 0.284 | 1.781 |
| mmu-miR-151-3p | 1 | 1 | −0.846 | 0.365 | 1.798 |
| mmu-miR-152 | 1 | 1 | n/a | n/a | n/a |
| mmu-miR-181a | 2 | 1 | n/a | n/a | n/a |
| mmu-miR-181c | 0 | 1 | n/a | n/a | n/a |
| mmu-miR-182 | 3 | 3 | −0.119 | 0.969 | 1.086 |
| mmu-miR-183 | 2 | 2 | −1.515 | 0.225 | 2.858 |
| mmu-miR-184 | 1 | 2 | −1.852 | 0.273 | 3.611 |
| mmu-miR-185 | 1 | 1 | n/a | n/a | n/a |
| mmu-miR-186 | 4 | 3 | 0.827 | 0.264 | −1.774 |
| mmu-miR-191 | 4 | 4 | 0.152 | 0.731 | −1.111 |
| mmu-miR-192 | 2 | 1 | 1.291 | 0.520 | −2.448 |
| mmu-miR-193b | 2 | 2 | −0.779 | 0.632 | 1.716 |
| mmu-miR-194 | 0 | 1 | n/a | n/a | n/a |
| mmu-miR-195 | 4 | 3 | 1.002 | 0.294 | −2.002 |
| mmu-miR-197 | 2 | 2 | 1.126 | 0.715 | −2.182 |
| mmu-miR-199a-3p | 1 | 2 | −2.219 | 0.176 | 4.655 |
| mmu-miR-200a | 0 | 2 | n/a | n/a | n/a |
| mmu-miR-200b | 1 | 0 | n/a | n/a | n/a |
| mmu-miR-200c | 2 | 2 | −2.973 | 0.096 | 7.851 |
| mmu-miR-203 | 1 | 0 | n/a | n/a | n/a |
| mmu-miR-204 | 0 | 2 | n/a | n/a | n/a |
| rno-miR-207 | 2 | 1 | 2.457 | 0.046 | −5.491 |
| mmu-miR-210 | 2 | 2 | −0.311 | 0.762 | 1.240 |
| mmu-miR-211 | 1 | 2 | −1.748 | 0.579 | 3.359 |
| mmu-miR-214 | 0 | 1 | n/a | n/a | n/a |
| snoRNA202 | 4 | 3 | 2.045 | 0.246 | −4.127 |
| mmu-miR-218 | 1 | 0 | n/a | n/a | n/a |
| mmu-miR-221 | 3 | 4 | −1.585 | 0.367 | 2.999 |
| mmu-miR-222 | 4 | 4 | −1.168 | 0.001 | 2.247 |
| mmu-miR-223 | 4 | 4 | 0.876 | 0.301 | −1.835 |
| mmu-miR-293 | 1 | 0 | n/a | n/a | n/a |
| mmu-miR-294 | 1 | 0 | n/a | n/a | n/a |
| mmu-miR-296-3p | 1 | 0 | n/a | n/a | n/a |
| mmu-miR-296-5p | 2 | 0 | n/a | n/a | n/a |
| mmu-miR-301a | 3 | 3 | 0.195 | 0.483 | −1.145 |
| mmu-miR-301b | 3 | 3 | 0.243 | 0.372 | −1.184 |
| mmu-miR-302a | 1 | 0 | n/a | n/a | n/a |
| mmu-miR-302d | 2 | 0 | n/a | n/a | n/a |
| mmu-miR-324-5p | 2 | 1 | 1.083 | 0.335 | −2.119 |
| rno-miR-327 | 2 | 1 | 2.120 | 0.157 | −4.346 |
| mmu-miR-328 | 3 | 3 | 0.103 | 0.887 | −1.074 |
| mmu-miR-331-3p | 3 | 4 | −1.927 | 0.332 | 3.801 |
| mmu-miR-331-5p | 1 | 2 | −0.716 | 0.817 | 1.643 |
| mmu-miR-335-5p | 0 | 1 | n/a | n/a | n/a |
| rno-miR-336 | 0 | 1 | n/a | n/a | n/a |
| mmu-miR-337-5p | 2 | 0 | n/a | n/a | n/a |
| mmu-miR-339-3p | 3 | 2 | 2.852 | 0.068 | −7.220 |
| mmu-miR-339-5p | 1 | 1 | n/a | n/a | n/a |
| U87 | 2 | 1 | n/a | n/a | n/a |
| mmu-miR-340-3p | 1 | 2 | −2.763 | 0.413 | 6.788 |
| mmu-miR-340-5p | 2 | 2 | 0.066 | 0.971 | −1.046 |
| mmu-miR-342-3p | 3 | 3 | −0.153 | 0.583 | 1.112 |
| mmu-miR-342-5p | 0 | 1 | n/a | n/a | n/a |
| rno-miR-345-3p | 1 | 1 | n/a | n/a | n/a |
| mmu-miR-345-5p | 1 | 1 | n/a | n/a | n/a |
| rno-miR-347 | 2 | 0 | n/a | n/a | n/a |
| mmu-miR-350 | 2 | 1 | 0.233 | 0.838 | −1.176 |
| rno-miR-351 | 3 | 1 | 1.823 | 0.419 | −3.538 |
| mmu-miR-361 | 0 | 1 | n/a | n/a | n/a |
| mmu-miR-362-3p | 0 | 1 | n/a | n/a | n/a |
| mmu-miR-363 | 2 | 1 | n/a | n/a | n/a |
| mmu-miR-365 | 4 | 3 | 0.854 | 0.709 | −1.807 |
| mmu-miR-375 | 2 | 0 | n/a | n/a | n/a |
| mmu-miR-376b | 1 | 0 | n/a | n/a | n/a |
| mmu-miR-376c | 0 | 1 | n/a | n/a | n/a |
| mmu-miR-381 | 2 | 2 | −1.528 | 0.518 | 2.884 |
| mmu-miR-409-3p | 3 | 2 | 0.617 | 0.764 | −1.534 |
| mmu-miR-410 | 1 | 0 | n/a | n/a | n/a |
| mmu-miR-411 | 2 | 1 | 1.078 | 0.317 | −2.111 |
| mmu-miR-423-5p | 2 | 2 | −0.732 | 0.722 | 1.661 |
| mmu-miR-425 | 3 | 3 | −0.352 | 0.903 | 1.276 |
| Y1 | 3 | 4 | 1.892 | 0.649 | −3.711 |
| mmu-miR-434-3p | 1 | 1 | n/a | n/a | n/a |
| mmu-miR-451 | 3 | 2 | 2.235 | 0.319 | −4.706 |
| mmu-miR-465a-5p | 1 | 0 | n/a | n/a | n/a |
| rno-miR-466b | 1 | 0 | n/a | n/a | n/a |
| mmu-miR-467c | 1 | 0 | n/a | n/a | n/a |
| mmu-miR-467d | 1 | 0 | n/a | n/a | n/a |
| mmu-miR-484 | 4 | 4 | −0.734 | 0.301 | 1.663 |
| mmu-miR-486 | 2 | 2 | n/a | n/a | n/a |
| mmu-miR-487b | 1 | 2 | n/a | n/a | n/a |
| mmu-miR-489 | 0 | 1 | n/a | n/a | n/a |
| mmu-miR-494 | 2 | 2 | n/a | n/a | n/a |
| mmu-miR-495 | 2 | 0 | n/a | n/a | n/a |
| mmu-miR-497 | 1 | 1 | n/a | n/a | n/a |
| mmu-miR-500 | 0 | 1 | n/a | n/a | n/a |
| mmu-miR-501-3p | 1 | 1 | n/a | n/a | n/a |
| mmu-miR-503 | 0 | 1 | n/a | n/a | n/a |
| mmu-miR-509-3p | 0 | 1 | n/a | n/a | n/a |
| mmu-miR-532-5p | 3 | 3 | −1.659 | 0.456 | 3.159 |
| rno-miR-532-5p | 1 | 1 | n/a | n/a | n/a |
| mmu-miR-539 | 1 | 2 | −3.369 | 0.318 | 10.329 |
| mmu-miR-542-3p | 0 | 1 | n/a | n/a | n/a |
| mmu-miR-574-3p | 2 | 3 | −2.487 | 0.350 | 5.607 |
| mmu-miR-615-3p | 0 | 1 | n/a | n/a | n/a |
| mmu-miR-615-5p | 2 | 1 | 1.100 | 0.413 | −2.143 |
| mmu-miR-654-5p | 0 | 1 | n/a | n/a | n/a |
| mmu-miR-665 | 1 | 0 | n/a | n/a | n/a |
| mmu-miR-671-3p | 4 | 2 | 2.246 | 0.102 | −4.742 |
| mmu-miR-672 | 1 | 0 | n/a | n/a | n/a |
| mmu-miR-674 | 1 | 2 | −1.575 | 0.053 | 2.978 |
| mmu-miR-708 | 1 | 0 | n/a | n/a | n/a |
| mmu-miR-741 | 0 | 1 | n/a | n/a | n/a |
| mmu-miR-743a | 2 | 2 | −1.065 | 0.413 | 2.092 |
| mmu-miR-744 | 3 | 2 | 0.771 | 0.708 | −1.706 |
| rno-miR-758 | 1 | 1 | n/a | n/a | n/a |
| mmu-miR-872 | 3 | 3 | 0.787 | 0.835 | −1.725 |
| mmu-miR-874 | 1 | 0 | n/a | n/a | n/a |
| mmu-miR-875-3p | 0 | 1 | n/a | n/a | n/a |
| rno-miR-878 | 0 | 1 | n/a | n/a | n/a |
| mmu-miR-881 | 0 | 1 | n/a | n/a | n/a |
| rno-miR-881 | 0 | 1 | n/a | n/a | n/a |
| mmu-miR-883a-3p | 0 | 1 | n/a | n/a | n/a |
| mmu-miR-143 | 1 | 2 | −2.054 | 0.398 | 4.153 |
| rno-miR-219-1-3p | 1 | 1 | n/a | n/a | n/a |
| rno-miR-224 | 1 | 0 | n/a | n/a | n/a |
| mmu-miR-324-3p | 3 | 2 | −0.887 | 0.159 | 1.850 |
| mmu-miR-351 | 3 | 1 | n/a | n/a | n/a |
| rno-miR-381 | 3 | 1 | 3.766 | 0.120 | −13.604 |
| mmu-miR-490 | 0 | 1 | n/a | n/a | n/a |
| mmu-miR-496 | 1 | 0 | n/a | n/a | n/a |
| mmu-miR-652 | 2 | 3 | −1.508 | 0.124 | 2.845 |
| mmu-miR-667 | 1 | 2 | n/a | n/a | n/a |
| mmu-miR-668 | 1 | 0 | n/a | n/a | n/a |
| mmu-miR-670 | 1 | 0 | n/a | n/a | n/a |
| mmu-miR-675-3p | 1 | 0 | n/a | n/a | n/a |
| mmu-miR-680 | 1 | 0 | n/a | n/a | n/a |
| mmu-miR-682 | 3 | 2 | 2.329 | 0.111 | −5.023 |
| mmu-miR-684 | 1 | 2 | −3.247 | 0.360 | 9.497 |
| mmu-miR-685 | 4 | 4 | 0.020 | 0.992 | −1.014 |
| mmu-miR-687 | 2 | 2 | −0.875 | 0.804 | 1.834 |
| mmu-let-7f | 0 | 1 | n/a | n/a | n/a |
| mmu-miR-106b | 3 | 2 | 1.727 | 0.269 | −3.311 |
| mmu-miR-155 | 4 | 4 | −7.512 | 0.072 | 182.508 |
| mmu-miR-17 | 4 | 4 | 0.000 | 0.999 | 1.000 |
| mmu-miR-23a | 1 | 1 | n/a | n/a | n/a |
| mmu-miR-29a | 4 | 4 | 0.365 | 0.684 | −1.288 |
| mmu-miR-30a | 3 | 3 | 0.293 | 0.101 | −1.225 |
| mmu-miR-30e | 4 | 3 | 1.412 | 0.417 | −2.661 |
| mmu-miR-31 | 4 | 3 | 4.501 | 0.394 | −22.636 |
| mmu-miR-34b-3p | 2 | 1 | 1.367 | 0.583 | −2.580 |
| mmu-miR-92a | 4 | 4 | −0.112 | 0.903 | 1.081 |
| rno-miR-190b | 3 | 3 | 2.078 | 0.641 | −4.224 |
| mmu-miR-106a | 4 | 4 | −2.937 | 0.284 | 7.656 |
| mmu-miR-188-5p | 3 | 4 | 1.374 | 0.703 | −2.593 |
| mmu-miR-322 | 2 | 2 | n/a | n/a | n/a |
| hsa-miR-136# | 1 | 1 | n/a | n/a | n/a |
| hsa-miR-30c-2# | 1 | 0 | n/a | n/a | n/a |
| hsa-miR-148a# | 1 | 0 | n/a | n/a | n/a |
| hsa-miR-33a# | 1 | 1 | n/a | n/a | n/a |
| hsa-miR-93# | 4 | 4 | 1.025 | 0.064 | −2.036 |
| hsa-miR-29b-2# | 0 | 1 | n/a | n/a | n/a |
| hsa-let-7i# | 3 | 1 | n/a | n/a | n/a |
| hsa-miR-15b# | 1 | 1 | −0.577 | 0.643 | 1.492 |
| hsa-miR-27b# | 3 | 1 | 1.979 | 0.260 | −3.941 |
| MammU6 | 4 | 4 | −0.325 | 0.219 | 1.253 |
| hsa-miR-935 | 0 | 1 | n/a | n/a | n/a |
| hsa-miR-99b# | 4 | 3 | 0.294 | 0.809 | −1.226 |
| hsa-miR-127-5p | 1 | 1 | n/a | n/a | n/a |
| hsa-miR-9# | 1 | 1 | n/a | n/a | n/a |
| hsa-miR-140-3p | 4 | 2 | 1.279 | 0.418 | −2.427 |
| hsa-miR-411# | 1 | 1 | n/a | n/a | n/a |
| hsa-miR-149 | 1 | 2 | −2.985 | 0.229 | 7.918 |
| hsa-miR-378 | 2 | 1 | 0.546 | 0.602 | −1.460 |
| mmu-miR-10b | 1 | 0 | n/a | n/a | n/a |
| hsa-miR-340 | 3 | 1 | 3.216 | 0.098 | −9.293 |
| hsa-miR-324-3p | 1 | 1 | −1.140 | 0.241 | 2.203 |
| hsa-miR-30e-3p | 4 | 3 | 1.723 | 0.270 | −3.300 |
| hsa-miR-30a-3p | 4 | 3 | 1.612 | 0.428 | −3.056 |
| hsa-miR-223 | 4 | 4 | −0.637 | 0.431 | 1.555 |
| hsa-miR-22 | 1 | 1 | −1.654 | 0.116 | 3.146 |
| hsa-miR-214 | 2 | 2 | −1.516 | 0.325 | 2.860 |
| hsa-miR-213 | 0 | 1 | n/a | n/a | n/a |
| hsa-miR-200c | 2 | 2 | −0.655 | 0.526 | 1.575 |
| hsa-miR-143 | 2 | 4 | −2.474 | 0.198 | 5.555 |
| hsa-miR-425 | 2 | 2 | −0.512 | 0.660 | 1.426 |
| mmu-miR-322 | 1 | 0 | n/a | n/a | n/a |
| mmu-miR-326 | 3 | 2 | −0.553 | 0.323 | 1.468 |
| mmu-miR-345 | 1 | 1 | −1.078 | 0.274 | 2.111 |
| hsa-miR-200b | 1 | 2 | −3.234 | 0.093 | 9.411 |
| mmu-miR-494 | 1 | 1 | n/a | n/a | n/a |
| mmu-miR-374-5p | 2 | 3 | 0.113 | 0.931 | −1.082 |
| mmu-miR-696 | 3 | 2 | −1.371 | 0.698 | 2.587 |
| mmu-miR-698 | 1 | 0 | n/a | n/a | n/a |
| mmu-miR-700 | 2 | 2 | −0.598 | 0.581 | 1.513 |
| mmu-miR-702 | 1 | 0 | n/a | n/a | n/a |
| mmu-miR-133a# | 1 | 0 | n/a | n/a | n/a |
| mmu-miR-706 | 4 | 4 | −0.347 | 0.507 | 1.272 |
| mmu-miR-710 | 0 | 1 | n/a | n/a | n/a |
| mmu-miR-721 | 1 | 2 | n/a | n/a | n/a |
| mmu-miR-690 | 3 | 1 | 0.287 | 0.506 | −1.220 |
| snoRNA135 | 3 | 2 | n/a | n/a | n/a |
| mmu-miR-692 | 2 | 3 | 0.268 | 0.892 | −1.204 |
| mmu-miR-694 | 3 | 2 | −0.427 | 0.672 | 1.345 |
| mmu-miR-467b | 3 | 3 | n/a | n/a | n/a |
| rno-miR-20b | 1 | 1 | n/a | n/a | n/a |
| rno-miR-350 | 2 | 2 | −0.818 | 0.451 | 1.763 |
| rno-miR-489 | 3 | 1 | 2.340 | 0.170 | −5.064 |
| rno-miR-29c# | 2 | 0 | n/a | n/a | n/a |
| hsa-miR-26b# | 1 | 0 | n/a | n/a | n/a |
| hsa-miR-27a# | 4 | 3 | 1.190 | 0.027 | −2.281 |
| hsa-miR-28-3p | 1 | 0 | n/a | n/a | n/a |
| hsa-miR-29a# | 2 | 1 | −1.597 | 0.578 | 3.026 |
| mmu-miR-761 | 1 | 0 | n/a | n/a | n/a |
| mmu-miR-763 | 1 | 0 | n/a | n/a | n/a |
| hsa-miR-23a# | 3 | 1 | 2.923 | 0.084 | −7.586 |
| mmu-miR-804 | 1 | 0 | n/a | n/a | n/a |
| mmu-miR-805 | 3 | 1 | 1.605 | 0.278 | −3.043 |
| mmu-miR-666-3p | 1 | 0 | n/a | n/a | n/a |
| mmu-miR-673-3p | 1 | 0 | n/a | n/a | n/a |
| mmu-miR-146b# | 3 | 2 | −1.303 | 0.310 | 2.467 |
| mmu-miR-130b# | 2 | 1 | 0.202 | 0.905 | −1.150 |
| mmu-miR-881# | 0 | 1 | n/a | n/a | n/a |
| mmu-miR-196a# | 2 | 0 | n/a | n/a | n/a |
| mmu-let-7a# | 1 | 1 | −1.205 | 0.210 | 2.306 |
| mmu-let-7c-1# | 2 | 1 | 0.706 | 0.754 | −1.632 |
| mmu-miR-16# | 0 | 1 | n/a | n/a | n/a |
| mmu-miR-18a# | 3 | 2 | 0.430 | 0.721 | −1.347 |
| mmu-miR-20a# | 2 | 1 | −0.267 | 0.817 | 1.203 |
| mmu-miR-24-2# | 3 | 3 | −1.454 | 0.256 | 2.740 |
| snoRNA202 | 3 | 2 | −0.188 | 0.898 | 1.139 |
| mmu-miR-29b# | 4 | 3 | 2.950 | 0.202 | −7.728 |
| mmu-miR-30b# | 1 | 1 | n/a | n/a | n/a |
| mmu-miR-322# | 1 | 1 | −1.500 | 0.130 | 2.829 |
| mmu-miR-125b# | 3 | 1 | 1.356 | 0.451 | −2.559 |
| mmu-miR-141# | 3 | 1 | 1.709 | 0.236 | −3.270 |
| mmu-miR-101b | 2 | 2 | −1.119 | 0.239 | 2.172 |
| mmu-miR-878-3p | 3 | 2 | 0.968 | 0.238 | −1.956 |
| mmu-miR-872# | 2 | 3 | −2.908 | 0.098 | 7.505 |
| mmu-miR-17# | 2 | 1 | −0.712 | 0.478 | 1.638 |
| mmu-miR-28# | 0 | 1 | n/a | n/a | n/a |
| mmu-miR-877# | 4 | 3 | 1.621 | 0.527 | −3.077 |
| mmu-miR-212 | 4 | 4 | −1.889 | 0.023 | 3.703 |
| mmu-miR-138# | 1 | 1 | n/a | n/a | n/a |
| mmu-miR-186# | 1 | 1 | −1.417 | 0.145 | 2.671 |
| mmu-miR-191# | 1 | 2 | −2.413 | 0.220 | 5.327 |
| mmu-miR-193# | 3 | 4 | −0.635 | 0.778 | 1.553 |
| mmu-miR-463# | 0 | 2 | n/a | n/a | n/a |
| mmu-miR-34c# | 3 | 1 | 2.456 | 0.105 | −5.485 |
| mmu-miR-470# | 2 | 2 | 0.852 | 0.688 | −1.805 |
| hsa-miR-493-3p | 1 | 0 | n/a | n/a | n/a |
| mmu-miR-487b | 1 | 1 | n/a | n/a | n/a |
| mmu-miR-449b | 1 | 1 | n/a | n/a | n/a |
| U87 | 2 | 2 | n/a | n/a | n/a |
| mmu-miR-467a | 0 | 1 | n/a | n/a | n/a |
| mmu-miR-485-3p | 1 | 0 | n/a | n/a | n/a |
| mmu-miR-673 | 4 | 4 | 0.796 | 0.487 | −1.736 |
| mmu-miR-674# | 3 | 3 | −1.004 | 0.238 | 2.006 |
| hsa-miR-190b | 3 | 2 | 0.013 | 0.983 | −1.009 |
| hsa-miR-183# | 2 | 3 | −1.971 | 0.269 | 3.920 |
| hsa-miR-10a# | 2 | 0 | n/a | n/a | n/a |
| hsa-miR-22# | 1 | 1 | −1.611 | 0.119 | 3.055 |
| hsa-miR-30d# | 1 | 1 | n/a | n/a | n/a |
| hsa-miR-744# | 1 | 1 | n/a | n/a | n/a |
| hsa-miR-106b# | 2 | 2 | −0.100 | 0.928 | 1.072 |
| hsa-let-7e# | 1 | 1 | n/a | n/a | n/a |
| mmu-miR-450B-3P | 1 | 1 | n/a | n/a | n/a |
| mmu-miR-712 | 4 | 4 | 0.067 | 0.955 | −1.048 |
| mmu-miR-465C-5P | 2 | 2 | 0.444 | 0.779 | −1.361 |
| mmu-miR-463 | 2 | 1 | 0.870 | 0.732 | −1.827 |
| mmu-miR-880 | 0 | 1 | n/a | n/a | n/a |
| mmu-miR-466E-5P | 4 | 4 | −0.235 | 0.913 | 1.177 |
| mmu-miR-1198 | 3 | 4 | −3.164 | 0.139 | 8.962 |
| mmu-miR-669D | 2 | 0 | n/a | n/a | n/a |
| mmu-miR-467H | 1 | 0 | n/a | n/a | n/a |
| mmu-miR-466J | 2 | 1 | 3.445 | 0.323 | −10.888 |
| mmu-miR-1195 | 3 | 1 | −0.414 | 0.676 | 1.332 |
| mmu-miR-1188 | 4 | 4 | −1.558 | 0.320 | 2.944 |
| mmu-miR-1-2-AS | 2 | 1 | −0.219 | 0.815 | 1.164 |
| mmu-miR-467F | 3 | 3 | 0.717 | 0.643 | −1.643 |
| rno-miR-409-3P | 0 | 2 | n/a | n/a | n/a |
| rno-miR-146B | 4 | 4 | −4.915 | 0.001 | 30.176 |
| hsa-miR-423-3P | 3 | 2 | 0.901 | 0.429 | −1.868 |
| hsa-miR-151-5P | 1 | 1 | n/a | n/a | n/a |
| hsa-miR-338-5P | 1 | 1 | n/a | n/a | n/a |
| Y1 | 4 | 3 | 1.203 | 0.797 | −2.301 |
| hsa-miR-421 | 3 | 3 | −0.680 | 0.807 | 1.603 |
| mmu-miR-362-5p | 2 | 1 | −0.217 | 0.811 | 1.163 |
| mmu-miR-34b-5p | 2 | 1 | 0.529 | 0.589 | −1.443 |
| rno-miR-743a | 3 | 3 | −0.115 | 0.948 | 1.083 |
| rno-miR-148b-5p | 1 | 1 | n/a | n/a | n/a |
| rno-miR-7a# | 3 | 3 | −0.970 | 0.320 | 1.959 |
| rno-miR-99a# | 2 | 2 | −2.243 | 0.208 | 4.733 |
| rno-miR-125b# | 1 | 2 | −2.596 | 0.200 | 6.046 |
| rno-miR-204# | 2 | 1 | n/a | n/a | n/a |
| rno-miR-29b-1# | 1 | 1 | n/a | n/a | n/a |
| hsa-miR-189 | 1 | 0 | n/a | n/a | n/a |
| mmu-miR-2138 | 4 | 4 | −0.064 | 0.956 | 1.045 |
| mmu-miR-2146 | 4 | 4 | 0.990 | 0.541 | −1.986 |
| mmu-miR-2183 | 3 | 2 | −0.114 | 0.972 | 1.082 |
| rno-miR-632 | 4 | 4 | −1.705 | 0.145 | 3.260 |
| mmu-miR-2182 | 3 | 3 | 0.792 | 0.525 | −1.732 |
| mmu-miR-2134 | 4 | 4 | −0.052 | 0.946 | 1.036 |
| mmu-miR-2139 | 0 | 1 | n/a | n/a | n/a |
| mmu-miR-2135 | 4 | 4 | 1.261 | 0.585 | −2.396 |
| hsa-miR-671-5p | 1 | 1 | n/a | n/a | n/a |
| hsa-miR-144 | 1 | 0 | n/a | n/a | n/a |
| mmu-miR-15a# | 1 | 1 | −1.117 | 0.252 | 2.169 |
| mmu-miR-503# | 0 | 1 | n/a | n/a | n/a |
| mmu-miR-1191 | 2 | 1 | −0.057 | 0.974 | 1.040 |
| mmu-miR-1904 | 4 | 3 | 0.882 | 0.731 | −1.843 |
| mmu-miR-297a# | 1 | 0 | n/a | n/a | n/a |
| mmu-miR-704 | 2 | 0 | n/a | n/a | n/a |
| mmu-miR-1274a | 4 | 4 | −0.315 | 0.772 | 1.244 |
| mmu-miR-1306 | 3 | 1 | 1.989 | 0.193 | −3.970 |
| mmu-miR-1839-3p | 3 | 2 | 0.852 | 0.694 | −1.805 |
| mmu-miR-1839-5p | 1 | 2 | −2.198 | 0.231 | 4.588 |
| mmu-miR-1896 | 4 | 3 | 2.786 | 0.220 | −6.899 |
| mmu-miR-1897-5p | 4 | 3 | 1.407 | 0.460 | −2.652 |
| mmu-miR-1898 | 1 | 1 | −1.478 | 0.134 | 2.787 |
| mmu-miR-1901 | 3 | 1 | 2.428 | 0.138 | −5.382 |
| mmu-miR-1905 | 3 | 3 | −1.670 | 0.495 | 3.183 |
| mmu-miR-1930 | 3 | 2 | 1.453 | 0.301 | −2.738 |
| mmu-miR-1932 | 2 | 1 | −0.691 | 0.541 | 1.615 |
| mmu-miR-1938 | 1 | 1 | n/a | n/a | n/a |
| mmu-miR-1940 | 1 | 1 | −1.138 | 0.242 | 2.200 |
| mmu-miR-1944 | 3 | 4 | −2.492 | 0.258 | 5.624 |
| mmu-miR-1951 | 3 | 4 | 1.669 | 0.441 | −3.181 |
| mmu-miR-1953 | 1 | 0 | n/a | n/a | n/a |
| mmu-miR-1954 | 1 | 1 | n/a | n/a | n/a |
| mmu-miR-1958 | 0 | 1 | n/a | n/a | n/a |
| mmu-miR-1959 | 1 | 1 | n/a | n/a | n/a |
| mmu-miR-1960 | 2 | 0 | n/a | n/a | n/a |
| mmu-miR-1969 | 3 | 2 | 0.924 | 0.391 | −1.897 |
| mmu-miR-1970 | 1 | 0 | n/a | n/a | n/a |
| mmu-miR-1971 | 4 | 4 | 1.435 | 0.445 | −2.704 |
| mmu-miR-1981 | 3 | 0 | n/a | n/a | n/a |
| mmu-miR-1982.1 | 2 | 2 | −1.231 | 0.600 | 2.347 |
| mmu-miR-1982.2 | 2 | 1 | −0.005 | 0.998 | 1.003 |
| mmu-miR-669l | 1 | 0 | n/a | n/a | n/a |
| mmu-let-7g# | 3 | 2 | 0.964 | 0.271 | −1.950 |
| mmu-miR-1186 | 1 | 0 | n/a | n/a | n/a |
| mmu-miR-669n | 1 | 2 | −1.066 | 0.739 | 2.093 |
| mmu-miR-1961 | 3 | 2 | −1.082 | 0.457 | 2.118 |
| mmu-miR-466k | 3 | 1 | 2.344 | 0.064 | −5.076 |
| mmu-miR-1894-3p | 3 | 2 | 1.593 | 0.521 | −3.017 |
| mmu-miR-1937c | 4 | 4 | 0.322 | 0.489 | −1.250 |
| mmu-miR-466g | 1 | 1 | n/a | n/a | n/a |
| mmu-miR-1937b | 4 | 4 | 0.020 | 0.893 | −1.014 |
| hsa-miR-875-5p | 3 | 2 | −0.558 | 0.890 | 1.472 |
| hsa-miR-206 | 2 | 2 | −0.044 | 0.927 | 1.031 |
| rno-miR-352 | 1 | 1 | n/a | n/a | n/a |
| rno-miR-7# | 4 | 4 | −1.805 | 0.097 | 3.495 |
| mmu-miR-21# | 1 | 4 | −6.834 | 0.021 | 114.067 |
| mmu-miR-466b-3-3p | 1 | 0 | n/a | n/a | n/a |
| mmu-miR-1194 | 1 | 0 | n/a | n/a | n/a |
| mmu-miR-1943 | 2 | 0 | n/a | n/a | n/a |
| mmu-miR-1949 | 3 | 2 | 0.236 | 0.892 | −1.178 |
| mmu-miR-1956 | 2 | 0 | n/a | n/a | n/a |
| mmu-miR-1946a | 1 | 0 | n/a | n/a | n/a |

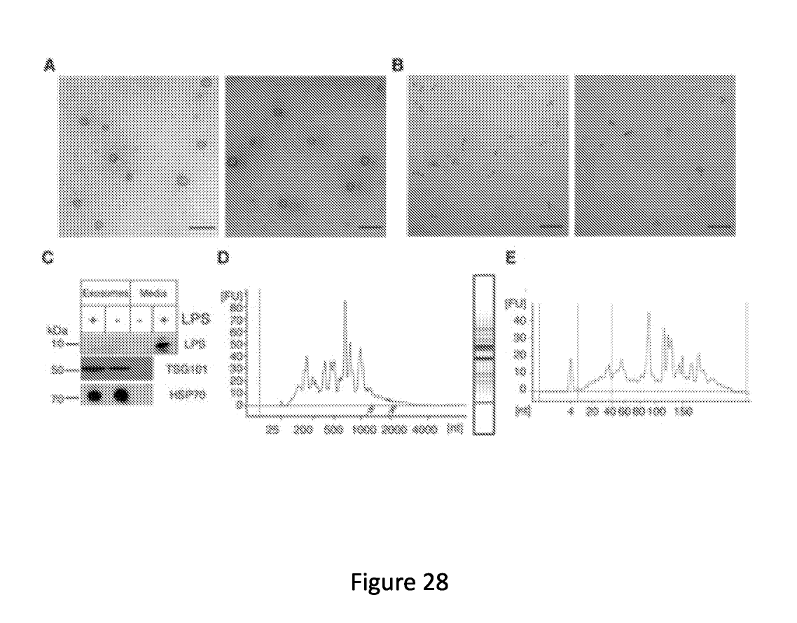

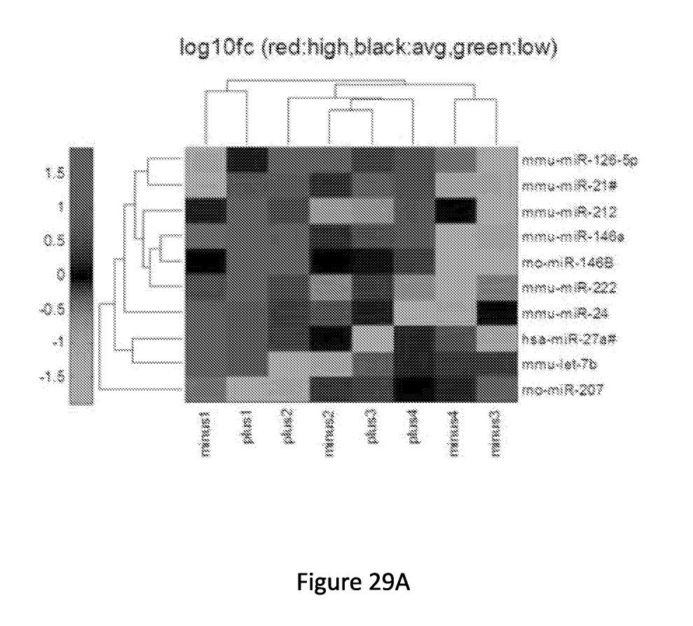

Ten miRNAs that were significantly altered after LPS treatment were studied further (FIG. 29A, Table 5). Significant alterations were also observed in THP1 cell-derived exosomes after LPS stimulation (FIGS. 29B and 29C).

TABLE 5
LPS-responsive exosomal miRNAs and verified mRNA targets
LPS-responsive exosomal miRNAs and verified mRNA targets. Statistical analysis of
433 detected miRNAs from LPS-treated and naive macrophages revealed 10 miRNAs that
were significantly altered after LPS stimulation (n = 4), including two homologues
that are also altered in patients with CRPS (bold). The fold change and P values are
reported as well as the validated mRNA targets of these miRNAs from miRTarBase.

| miRNA | Fold change | P value | Validated human mRNA targets |
|---|---|---|---|
| mmu-miR-21-3p | 114.067 | 0.0207 | Seed sequence is slightly different in human miR-21-3p |
| rno-miR-146b | 30.1762 | 0.0009 | CDKN1A, IRAK1, KIT, MMP16, NF-KB1, PDGFB, |
| | | | TRAF6 |
| mmu-miR-146a | 9.8140 | 0.0190 | BRCA1 & 2, CCNA2, CD40LG, CDKN1A, CDKN3, CFH, |
| | | | CXCR4, EGFR, ERBB4, FADD, FAS, FAF1, IL-8, IRAK |
| | | | 1 & 2, KIF22, MTA2, NF-KB1, PA2G4, ROCK1, SMAD4, |
| | | | TLR2, TLR4, TRAF6, WASF2 |
| mmu-miR-126-5p | 5.8577 | 0.0084 | ADAM9, CXCL12, MMP7, PTPN7, SLC45A3 |
| mmu-miR-212 | 3.7034 | 0.0229 | CCNA2, CCNB1, KCNJ2, MECP2, PEA15, PTCH1, |
| | | | RB1, TJP1 |
| mmu-miR-222 | 2.2474 | 0.0009 | BBC3, CDKN1B/C, CERS2, CORO1A, DICER, DIRAS3, |
| | | | ESR1, ETS1, FOXO3, ICAM1, KIT, MMP1, PPP2R2A, |
| | | | PTEN, RECK, SELE, SOD2, STAT5, TIMP3, TMED7, |
| | | | TNFSF10, TP53, TRPS1 |
| mmu-miR-24 | −1.3151 | 0.0486 | ACVR1B, AURKB, BRCA1, CCNA2, CDK4, CDKN1B, |
| | | | CDKN2A, DND1, DHFR, E2F2, FAF1, FEN1, FURIN, |
| | | | HNF4A, MAPK14, MYC, NOTCH1, POLD1, ST7L, |
| | | | TGFB1, TRIB3, VSIVgp5, ZNF217 |
| rno-miR-27a-5p | −2.2801 | 0.0272 | No validated targets for rare form |
| mmu-let-7b | −3.2575 | 0.0367 | ACTG1, CCNA1/2, CCND1/2, CDC25A, CDC34, CDK6, |
| | | | CYP2J2, HMGA2, IFNB1, LIN28A/B, MTPN, NR2E1, |
| | | | NRAS, PRDM1, RDH10, RPIA |
| rno-miR-207 | −5.4906 | 0.0462 | N/A No human homologue |

This subset of LPS-responsive miRNAs included 2 miRNAs that are dysregulated in CRPS (miR-126-5p and miR-let7b) and 3 miRNAs previously reported to be upregulated in cells after LPS treatment (miR-146a, miR-146b, and miR-21-3p) (Bhaumik et al., 2008, Oncogene 2008; 27(42):5643-5647; Taganov et al., 2006, Proc Natl Acad Sci USA 103(33):12481-12486). Many of the LPS-responsive miRNAs that we detected in exosomes have validated mRNA targets that encode proteins involved in TLR signaling, chemokine signaling, and the TGF-P pathway (Hsu et al., 2011, Nucleic acids research 39(Database issue):D163-169).

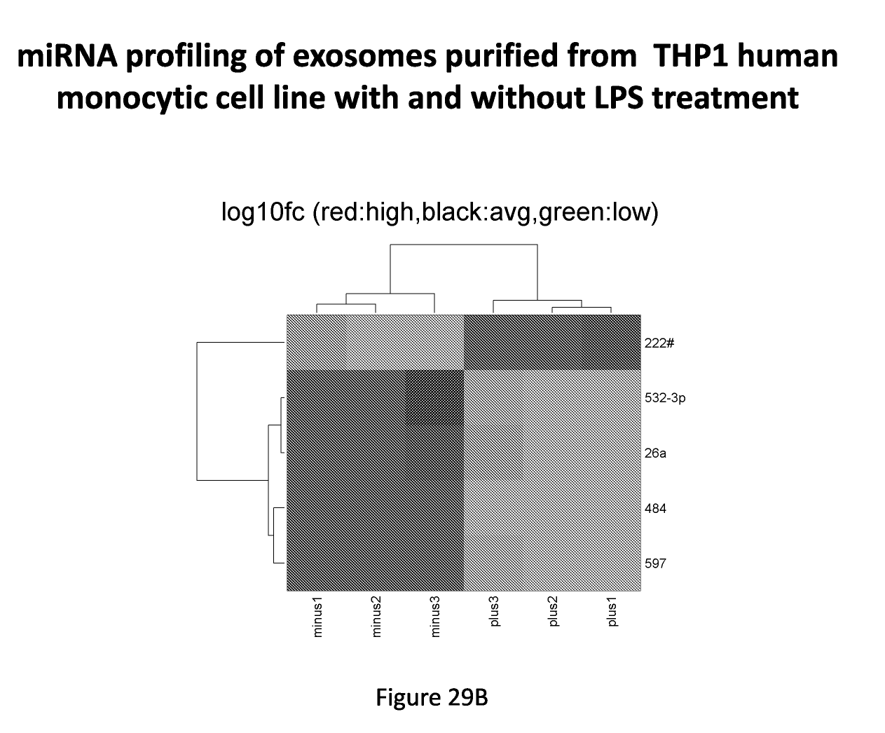

To identify the transcriptome secreted in exosomes from naive or LPS-stimulated RAW 264.7 cells, next-generation sequencing (NGS) was performed. Total reads were mapped from naive (10323145 reads) and LPS treated (9418995 reads) cells, respectively. A total of 15883 genes were matched to the mouse genome. Significant differences were observed between naive and LPS-stimulated exosomal RNA (FIGS. 30A and 30B). Of the 15,883 unique exosomal transcripts, 3559 mapped to noncoding or unverified sequences and the remaining 12,324 were analyzed for differential regulation after LPS stimulation (FIG. 30C). Of the 7142 transcripts encoding proteins found in exosomes, 3351 were specific to unstimulated exosomes. Additionally, 1632 genes were found only in LPS-stimulated exosomes and 1271 were differentially expressed after LPS stimulation (Table 6).

TABLE 6
LPS-responsive exosomal mRNAs
Analysis of exosomal mRNA levels after LPS stimulation of RAW 264.7 cells compared with
exosomes from naive cells revealed that transcripts known to be involved in pain and
inflammation are upregulated and transported in exosomes. All genes with greater than 2-fold
increase after LPS stimulation are reported.

| | | Ensembl | Entrez | FKPM | KPM | PS/cpntrol |
|---|---|---|---|---|---|---|
| GeneSymbol | Locus | GeneID | Gene | LPS | control | ratio |
| Mir155 | hr16: 84714384-84714449 | NSMUSG00000065397 | A | 75874 | 28.592 | 39.56 |
| Ctdsp2, Mir26a- | hr10: 126415772-126437031 | A | A | 793.1 | .30434 | 78.14 |
| 2, Mir546 | | | | | | |
| Mir3082, Stub1 | hr17: 25965987-25970306 | A | A | 35.424 | .590414 | 68.12 |
| Frmd6 | hr12: 71926500-72003221 | NSMUSG00000048285 | 19710 | 18.996 | .239359 | 97.14 |
| Dgcr8, Mir1306 | hr16: 18248975-18289261 | A | A | 319.28 | .41407 | 86.42 |
| Rnf217 | hr10: 31221692-31329634 | NSMUSG00000063760 | 68291 | 8.8912 | .128185 | 03.40 |
| Mir700, Rcan3 | hr4: 134968223-134989720 | A | A | 91.844 | .50989 | 25.60 |
| Hipk3, Mir1902 | hr2: 104266638-104334646 | A | A | 52.273 | .96505 | 64.51 |
| Cyp4f16 | hr17: 32673573-32688742 | NSMUSG00000048440 | 0101 | 1.7633 | .152186 | 43.00 |
| FR197770, Mir22, Mir22hg | hr11: 75275040-75280192 | A | A | 129.35 | .04149 | 40.44 |
| Snord16a | hr9: 64021193-64026369 | A | A | 481.93 | 0.5894 | 10.42 |
| FR324012 | hr2: 38578893-38781981 | A | A | 196.2 | 9.8886 | 03.46 |
| Mir135a-2 | hr10: 91534830-91534930 | NSMUSG00000065524 | A | 550.1 | 6.6399 | 9.60 |
| Slc35f1 | hr10: 52410306-52831428 | NSMUSG00000038602 | 15085 | .73792 | .0476988 | 7.40 |
| Btbd11 | hr10: 84849558-85123037 | NSMUSG00000020042 | 4007 | .83992 | .0731279 | 2.51 |
| Jag2 | hr12: 114146800-114167706 | NSMUSG00000002799 | 6450 | .754176 | .0153974 | 8.98 |
| Mylk4 | hr13: 32792695-32873648 | NSMUSG00000044951 | 38564 | .59527 | .0328381 | 8.58 |
| 2900060B14Rik | hr1: 120285634-120506039 | NSMUSG00000073596 | A | 4.51 | .328908 | 4.12 |
| Pkd2l1 | hr19: 44222126-44266932 | NSMUSG00000037578 | 29064 | .09087 | .0255968 | 2.62 |
| Cspg4 | hr9: 56712910-56747677 | NSMUSG00000032911 | 21021 | .41267 | .00997146 | 1.39 |
| Serpinb2 | hr1: 109407999-109422177 | NSMUSG00000062345 | 8788 | .17245 | .0527606 | 1.18 |
| Mt2 | hr8: 96696517-96697467 | NSMUSG00000031762 | 7750 | 1.6862 | .546913 | 9.65 |
| Rdh8 | hr9: 20622947-20630607 | NSMUSG00000053773 | 35033 | .50642 | .0633443 | 9.57 |
| Vcam1 | hr3: 115812937-115832606 | NSMUSG00000027962 | 2329 | .91645 | .100899 | 8.82 |
| Shisa6 | hr11: 66025226-66339628 | NSMUSG00000053930 | 80702 | .541902 | .0141846 | 8.20 |
| Rogdi | hr16: 5008821-5013646 | NSMUSG00000022540 | 6049 | .81069 | .0738971 | 8.04 |
| Rabggtb, Snord45c | hr3: 153570252-153575930 | A | A | 94.325 | .19129 | 7.43 |
| Mir719, Nupl1 | hr14: 60838304-60870215 | A | A | 6.2099 | .38953 | 6.08 |
| Psme2 | hr14: 56206276-56209938 | NSMUSG00000079197 | 9188 | .64227 | .143461 | 2.36 |
| Dync1i2 | hr2: 71049762-71101359 | NSMUSG00000027012 | 3427 | .06356 | .0331404 | 2.09 |
| Zfp426 | hr9: 20272992-20297190 | NSMUSG00000059475 | 35028 | .877302 | .028947 | 0.31 |
| FR176580 | hr13: 63402007-63402104 | A | A | 16.842 | 0.4871 | 0.11 |
| Il1rn | hr2: 24192379-24207011 | NSMUSG00000026981 | 6181 | .73919 | .0605061 | 8.74 |
| Mylk2 | hr2: 152737087-152748801 | NSMUSG00000027470 | 28785 | .84071 | .0305315 | 7.54 |
| Pde8b | hr13: 95794059-96020259 | NSMUSG00000021684 | 18461 | .5638 | .020596 | 7.37 |
| Gnas | hr2: 174106737-174172243 | NSMUSG00000027523 | 4683 | .05456 | .0766468 | 6.81 |
| Tspo | hr15: 83394002-83404633 | NSMUSG00000041736 | 2257 | .81673 | .143665 | 6.57 |
| Rapgef5 | hr12: 118754951-118995451 | NSMUSG00000041992 | 17944 | .526941 | .0205482 | 5.64 |
| Mirlet7c-2 | hr15: 85537032-85537127 | NSMUSG00000065608 | A | 055.31 | 1.7475 | 5.14 |
| Snord65 | hr11: 62416378-62418308 | NSMUSG00000077457 | A | 590.04 | 44.332 | 4.95 |
| Ikzf2 | hr1: 69577783-69732534 | NSMUSG00000025997 | 2779 | .300375 | .012041 | 4.95 |
| Klhl8 | hr5: 104291068-104340248 | NSMUSG00000029312 | 46293 | .748551 | .031411 | 3.83 |
| Mir874 | hr13: 58106317-58203789 | NSMUSG00000077962 | A | 500 | 58.255 | 3.73 |
| Vwa5b1 | hr4: 138124883-138179907 | NSMUSG00000028753 | 5718 | .447032 | .0189613 | 3.58 |
| Cep72 | hr13: 74173942-74199733 | NSMUSG00000021572 | 4470 | .576815 | .0245128 | 3.53 |
| A630001G21Rik | hr1: 87613657-87633181 | NSMUSG00000052760 | 19997 | .47428 | .0628482 | 3.46 |
| Ola1 | hr2: 72930857-73052504 | NSMUSG00000027108 | 7059 | .65981 | .114558 | 3.22 |
| Pgcp | hr15: 33012883-33583191 | NSMUSG00000039007 | 4381 | .17431 | .138363 | 2.94 |
| Ppapdc2 | hr19: 28997553-29041291 | NSMUSG00000040105 | 4411 | .88242 | .0395238 | 2.33 |
| Sema6a | hr18: 47404907-47528522 | NSMUSG00000019647 | 0358 | .309989 | .0139807 | 2.17 |
| Prdm12 | hr2: 31495556-31511315 | NSMUSG00000079466 | 81359 | .11181 | .0503582 | 2.08 |
| Mir135a-1 | hr9: 106055190-106060469 | NSMUSG00000065407 | A | 261.71 | 7.6865 | 1.87 |
| Arid3b | hr9: 57638315-57682041 | NSMUSG00000004661 | 6380 | .421867 | .0194002 | 1.75 |
| Lace1 | hr10: 42032390-42198371 | NSMUSG00000038302 | 15951 | .792426 | .0364799 | 1.72 |
| Tmco4 | hr4: 138528819-138615086 | NSMUSG00000041143 | 7056 | .529799 | .0247379 | 1.42 |
| Dpagt1 | hr9: 44134927-44141683 | NSMUSG00000032123 | 3478 | .44034 | .0673658 | 1.38 |
| Marcks | hr10: 36853048-36858732 | NSMUSG00000069662 | 7118 | .686251 | .0321001 | 1.38 |
| Neurl3 | hr1: 36321446-36330270 | NSMUSG00000047180 | 14854 | .891081 | .041705 | 1.37 |
| Vwa8 | hr14: 79248984-79602117 | A | A | .06962 | .0513993 | 0.81 |
| Mir146 | hr11: 43187898-43187963 | NSMUSG00000065601 | A | 5951.3 | 269.5 | 0.44 |
| Ndufa7 | hr17: 33961558-33975258 | NSMUSG00000041881 | 6416 | .56292 | .468017 | 0.43 |
| Zfp964 | hr8: 72178454-72188352 | NSMUSG00000091764 | 36741 | .28812 | .112375 | 0.36 |
| Col6a6 | hr9: 105591746-105730415 | NSMUSG00000043719 | 45026 | .875632 | .0432235 | 0.26 |
| NA | hr1: 137550592-137552005 | A | A | .66592 | .0826234 | 0.16 |
| BC048355 | hr17: 46633737-46636567 | NSMUSG00000040658 | 81101 | .20453 | .216044 | 9.46 |
| NA | hr15: 102748149-102748333 | A | A | 71.768 | .83458 | 9.44 |
| Dock6 | hr9: 21604623-21657079 | NSMUSG00000032198 | 19899 | .258497 | .0134335 | 9.24 |
| Pld1 | hr3: 27837601-28032284 | NSMUSG00000027695 | 8805 | .323397 | .0168412 | 9.20 |
| Prr15l | hr11: 96790637-96796961 | NSMUSG00000047040 | 17138 | .79768 | .0941634 | 9.09 |
| Dusp18 | hr11: 3795242-3801299 | NSMUSG00000047205 | 5219 | .431745 | .0227303 | 8.99 |
| Dnajc1 | hr2: 17976863-18314457 | NSMUSG00000026740 | 3418 | .67311 | .201318 | 8.25 |
| Zmym2 | hr14: 57506631-57581416 | NSMUSG00000021945 | 6007 | .48292 | .0265251 | 8.21 |
| Slc4a4 | hr5: 89316284-89668681 | NSMUSG00000060961 | 4403 | .228277 | .0127813 | 7.86 |
| Nos2 | hr11: 78734360-78773626 | NSMUSG00000020826 | 8126 | .369028 | .0209285 | 7.63 |
| Lrfn5 | hr12: 62625618-62946245 | NSMUSG00000035653 | 38205 | .886864 | .051308 | 7.29 |
| Mir532 | hrX: 6735537-6896484 | NSMUSG00000070139 | A | 43.42 | 1.4626 | 7.27 |
| Olfr1311 | hr2: 111861070-111862009 | NSMUSG00000074948 | 58271 | 52.025 | .27491 | 6.39 |
| Ncapg2 | hr12: 117643874-117702004 | NSMUSG00000042029 | 6044 | .233483 | .0147528 | 5.83 |
| Spon1 | hr7: 120909511-121186889 | NSMUSG00000038156 | 33744 | .221508 | .0140779 | 5.73 |
| Ccdc107 | hr4: 43506236-43508793 | NSMUSG00000028461 | 22404 | .78698 | .113957 | 5.68 |
| Zfp11 | hr5: 130160469-130175963 | NSMUSG00000051034 | 2648 | .85884 | .0554706 | 5.48 |
| Narg2 | hr9: 69245804-69280881 | NSMUSG00000032235 | 3697 | .371577 | .0240082 | 5.48 |
| Mir26b | hr1: 74438182-74443859 | NSMUSG00000065468 | A | 6264.3 | 705.11 | 5.40 |
| St6galnac2 | hr11: 116538018-116555974 | NSMUSG00000057286 | 0446 | .76812 | .179877 | 5.39 |
| Cog7 | hr7: 129066352-129125207 | NSMUSG00000034951 | 33824 | .921639 | .0601989 | 5.31 |
| Rtn4rl1 | hr11: 75007494-75081264 | NSMUSG00000045287 | 37847 | .43479 | .0937414 | 5.31 |
| Col20a1 | hr2: 180721239-180752245 | NSMUSG00000016356 | 3368 | .987275 | .0645635 | 5.29 |
| Cinp | hr12: 112110819-112127355 | NSMUSG00000021276 | 40972 | .65093 | .108882 | 5.16 |
| Aldoart2 | hr12: 56666121-56667696 | NSMUSG00000063129 | A | .09318 | .072208 | 5.14 |
| Csnk2b | hr17: 35253139-35258392 | NSMUSG00000024387 | 3001 | .58541 | .106919 | 4.83 |
| Gdf11 | hr10: 128321601-128328774 | NSMUSG00000025352 | 4561 | .905503 | .0610812 | 4.82 |
| Sox13 | hr1: 135278876-135320789 | NSMUSG00000070643 | 0668 | .362688 | .0246317 | 4.72 |
| Sfxn5 | hr6: 85163044-85283416 | NSMUSG00000033720 | 4282 | .422308 | .0287418 | 4.69 |
| Dram2 | hr3: 106350744-106377763 | NSMUSG00000027900 | 7171 | .03709 | .0708987 | 4.63 |
| Ovgp1 | hr3: 105776719-105790341 | NSMUSG00000074340 | 2659 | .474722 | .0324603 | 4.62 |
| Il12rb1 | hr8: 73332347-73345322 | NSMUSG00000000791 | 6161 | .587649 | .0402257 | 4.61 |
| Dnm3os, FR136357 | hr1: 163917432-164408165 | A | A | 1339.8 | 147.95 | 4.59 |
| Prpsap2 | hr11: 61543151-61575590 | NSMUSG00000020528 | 12627 | .831652 | .057098 | 4.57 |
| FR303592 | hr6: 124667926-124668007 | A | A | 217.6 | 56.301 | 4.19 |
| Rhbdd2 | hr5: 136108523-136122246 | NSMUSG00000039917 | 15160 | .234044 | .0165232 | 4.16 |
| Necap2 | hr4: 140622426-140634260 | NSMUSG00000028923 | 6147 | .65694 | .118737 | 3.95 |
| Mir7-1 | hr13: 58493311-58512510 | NSMUSG00000065434 | A | 101.11 | 9.2247 | 3.90 |
| Epb4.9 | hr14: 71001990-71035855 | NSMUSG00000022099 | 3829 | .357589 | .0258015 | 3.86 |
| Oscp1 | hr4: 125735808-125766578 | NSMUSG00000042616 | 00044374 | .612667 | .0448591 | 3.66 |
| Mir26a-1 | hr9: 118835653-118953237 | NSMUSG00000065513 | A | 4914.4 | 095.48 | 3.61 |
| Emc4 | hr2: 112106470-112208184 | A | A | .25635 | .0927274 | 3.55 |
| Usp16 | hr16: 87455229-87496114 | NSMUSG00000025616 | 4112 | .421332 | .0311125 | 3.54 |
| Mir345 | hr12: 110075182-110075278 | NSMUSG00000065429 | A | 463.23 | 08.703 | 3.46 |
| Phospho2 | hr2: 69627793-69635225 | NSMUSG00000027088 | 3373 | .788517 | .0587665 | 3.42 |
| Pdss2 | hr10: 42941291-43184688 | NSMUSG00000038240 | 1365 | .791507 | .0593123 | 3.34 |
| Cacnb1 | hr11: 97864214-97883941 | NSMUSG00000020882 | 2295 | .676801 | .0507281 | 3.34 |
| Ccdc28b | hr4: 129296517-129301152 | NSMUSG00000028795 | 6264 | .61628 | .121293 | 3.33 |
| Mir106b | hr5: 138605816-138613090 | NSMUSG00000065514 | A | 748.28 | 32.974 | 3.15 |
| Arrdc1 | hr2: 24780871-24790801 | NSMUSG00000026972 | 15705 | .07994 | .160072 | 2.99 |
| Ufsp2 | hr8: 47060892-47082213 | NSMUSG00000031634 | 92169 | .654524 | .0503861 | 2.99 |
| Gemin7 | hr7: 20150297-20158692 | NSMUSG00000044709 | 9731 | .60576 | .354937 | 2.98 |
| FR250891 | hr12: 110828378-110833613 | A | A | 17.835 | 2.7562 | 2.62 |
| Mir150 | hr7: 52377126-52377191 | NSMUSG00000065495 | A | 1858700 | 341350 | 2.53 |
| Rasgrp2 | hr19: 6400582-6415216 | NSMUSG00000032946 | 9395 | .80154 | .223655 | 2.53 |
| LOC171588 | hr2: 144285015-144293530 | A | A | .690932 | .0551617 | 2.53 |
| Chsy3 | hr18: 59334993-59570990 | NSMUSG00000058152 | 8923 | .275891 | .022058 | 2.51 |
| Snhg6 | hr1: 9932105-9934199 | A | A | .80082 | .303897 | 2.51 |
| Ralbp1 | hr17: 66197768-66235095 | NSMUSG00000024096 | 9765 | .42288 | .433767 | 2.50 |
| Brdt | hr5: 107760212-107816077 | NSMUSG00000029279 | 14642 | .191802 | .0153862 | 2.47 |
| Traf1 | hr2: 34798777-34817292 | NSMUSG00000026875 | 2029 | .43194 | .277253 | 2.38 |
| 5-Sep | hrX: 34450828-34529690 | NSMUSG00000050379 | 6526 | .78358 | .063529 | 2.33 |
| Hvcn1 | hr5: 122659745-122714469 | NSMUSG00000064267 | 4096 | .50343 | .0408644 | 2.32 |
| Oxgr1 | hr14: 120418806-120441657 | NSMUSG00000044819 | 39283 | .86146 | .151206 | 2.31 |
| Srcin1 | hr11: 97370653-97436440 | NSMUSG00000038453 | 6013 | .173805 | .0142138 | 2.23 |
| Ppih | hr4: 118972614-118993128 | NSMUSG00000060288 | 33064 | .536269 | .0442565 | 2.12 |
| Ppap2c | hr10: 78989173-78996532 | NSMUSG00000052151 | 0784 | .95519 | .16142 | 2.11 |
| Vps41 | hr13: 18809160-18958680 | NSMUSG00000041236 | 18035 | .599443 | .0495612 | 2.10 |
| Camk2d | hr3: 126299890-126547972 | NSMUSG00000053819 | 08058 | .640682 | .0537075 | 1.93 |
| Snrpd2 | hr7: 19735186-19738075 | NSMUSG00000040824 | 45531 | 1.9868 | .00951 | 1.87 |
| Lefty1 | hr1: 182865169-182868532 | NSMUSG00000038793 | 3590 | .637737 | .0538342 | 1.85 |
| Elmod1 | hr9: 53759266-53823108 | NSMUSG00000041986 | 70162 | .971638 | .083319 | 1.66 |
| Slc2a13 | hr15: 91098121-91403692 | NSMUSG00000036298 | 39606 | .10211 | .267073 | 1.62 |
| NA | hr3: 41214830-41216268 | A | A | .932706 | .0809528 | 1.52 |
| Zfp383 | hr7: 30693535-30701832 | NSMUSG00000058402 | 33058 | .430958 | .0378008 | 1.40 |
| Rasl2-9 | hr7: 5076543-5077552 | NSMUSG00000083649 | 9428 | .14537 | .101571 | 1.28 |
| Mir31 | hr4: 88556460-88556566 | NSMUSG00000065408 | A | 32.749 | 9.5643 | 1.26 |
| Slc29a3 | hr10: 60174819-60215530 | NSMUSG00000020100 | 1279 | .416018 | .0370432 | 1.23 |
| H2-M10.5 | hr17: 36909854-36913180 | NSMUSG00000037246 | 24761 | .915597 | .0815815 | 1.22 |
| Cpm | hr10: 117066555-117124408 | NSMUSG00000020183 | 0574 | .238523 | .021698 | 0.99 |
| Sms | hrX: 153881885-153929978 | NSMUSG00000071708 | 71878 | .339498 | .0310856 | 0.92 |
| FR019097 | hr1: 195333642-195333750 | A | A | 1075.3 | 021.31 | 0.84 |
| Ets1 | hr9: 32503626-32565405 | NSMUSG00000032035 | 3871 | .247606 | .0230624 | 0.74 |
| Suclg2 | hr6: 95424127-95668831 | NSMUSG00000061838 | 0917 | .500186 | .0467232 | 0.71 |
| Trim30a | hr7: 111557539-111613707 | NSMUSG00000030921 | 0128 | .619759 | .0586357 | 0.57 |
| Nutf2 | hr8: 108384533-108404301 | NSMUSG00000008450 | 68830 | .587899 | .0556991 | 0.55 |
| Copz1 | hr15: 103103348-103130295 | NSMUSG00000060992 | 6447 | .96449 | .186469 | 0.54 |
| Atp6v0d1 | hr8: 108048369-108089940 | NSMUSG00000013160 | 1972 | .19005 | .113442 | 0.49 |
| Ankle2 | hr5: 110660022-110685670 | NSMUSG00000029501 | 1782 | .171211 | .0163294 | 0.48 |
| Olr1 | hr6: 129435265-129457183 | NSMUSG00000030162 | 08078 | .289441 | .0278239 | 0.40 |
| D330045A20Rik | hrX: 136014905-136089119 | NSMUSG00000042498 | 02871 | .291661 | .0282028 | 0.34 |
| Grik1 | hr16: 87896141-88290503 | NSMUSG00000022935 | 4805 | .221528 | .021535 | 0.29 |
| Mrps36 | hr13: 101505894-101514614 | NSMUSG00000061474 | 6128 | .75918 | .270397 | 0.20 |
| Chrnb3 | hr8: 28479182-28510202 | NSMUSG00000031492 | 08043 | .241211 | .023674 | 0.19 |
| 1700084J12Rik | hr15: 33012883-33583191 | NSMUSG00000058101 | A | .33336 | .229236 | 0.18 |
| Hlx | hr1: 186551023-186556372 | NSMUSG00000039377 | 5284 | .508496 | .0499609 | 0.18 |
| H2-Q7 | hr17: 35576099-35580718 | NSMUSG00000060550 | 10558 | .33118 | .130976 | 0.16 |
| FR345013 | hr18: 61799306-61825192 | A | A | 6478.2 | 606.87 | 0.11 |
| Wrn | hr8: 34344844-34495999 | NSMUSG00000031583 | 2427 | .308527 | .0305525 | 0.10 |
| Ahr | hr12: 36182650-36219661 | NSMUSG00000019256 | 1622 | .187596 | .0186601 | 0.05 |
| Npy2r | hr3: 82342304-82352007 | NSMUSG00000028004 | 8167 | 07.761 | 0.6221 | 0.05 |
| Ifi44 | hr3: 151393886-151412923 | NSMUSG00000028037 | 9899 | .415444 | .0417858 | .94 |
| Zfp955b | hr17: 33426488-33441634 | NSMUSG00000096910 | 00043468 | .280043 | .02817 | .94 |
| 4932412D23Rik | hr16: 42725814-42875875 | NSMUSG00000075070 | A | .0144 | .705624 | .94 |
| Actr10 | hr12: 72038843-72065704 | NSMUSG00000021076 | 6444 | .5514 | .0557249 | .90 |
| Mir100 | hr9: 41339507-41339587 | NSMUSG00000093011 | A | 5841.2 | 686.77 | .89 |
| Chst7 | hrX: 19636695-19674646 | NSMUSG00000037347 | 0322 | .573501 | .0580656 | .88 |
| Tob1 | hr11: 94072767-94076806 | NSMUSG00000037573 | 2057 | .36578 | .139519 | .79 |
| Htatsf1 | hrX: 54306746-54320359 | NSMUSG00000067873 | 2459 | .263949 | .0269676 | .79 |
| Mirlet7a-2 | hr9: 41344798-41344894 | NSMUSG00000092770 | A | 1719.1 | 244.12 | .78 |
| Pramel6 | hr2: 87348614-87351022 | NSMUSG00000025838 | 47711 | .10201 | .523425 | .75 |
| Nsmce4a | hr7: 137676039-137690895 | NSMUSG00000040331 | 7872 | .98038 | .203267 | .74 |
| Ddhd1, Mir5131 | hr14: 46212848-46277818 | A | A | .190955 | .0198358 | .63 |
| DXBay18_dup1, | hrX: 70352973-70374599 | A | A | .266508 | .0277748 | .60 |
| Gm14685_dup1 | | | | | | |
| Nkpd1 | hr7: 20104079-20110399 | NSMUSG00000060621 | 9547 | .14474 | .120073 | .53 |
| Kcna5 | hr6: 126482568-126485573 | NSMUSG00000045534 | 6493 | .323352 | .0341997 | .45 |
| Sp7 | hr15: 102187607-102196702 | NSMUSG00000060284 | 70574 | .46347 | .366639 | .45 |
| Xdh | hr17: 74233247-74299522 | NSMUSG00000024066 | 2436 | .201896 | .0213758 | .45 |
| Nfkbiz | hr16: 55811489-55838754 | NSMUSG00000035356 | 0859 | .70898 | .287885 | .41 |
| Zfp653 | hr9: 21859903-21890575 | NSMUSG00000038895 | 19601 | .385375 | .0410498 | .39 |
| NA | hr6: 31068761-31069903 | A | A | .9927 | .212865 | .36 |
| Prss1 | hr6: 41408928-41413785 | NSMUSG00000062751 | 14228 | .28402 | .137176 | .36 |
| Cep63 | hr9: 102488907-102528454 | NSMUSG00000032534 | 8135 | .2422 | .0258879 | .36 |
| Pml | hr9: 58064986-58097593 | NSMUSG00000036986 | 8854 | .325997 | .0349827 | .32 |
| Coq5 | hr5: 115729710-115746981 | NSMUSG00000041733 | 2064 | .00926 | .108745 | .28 |
| Arl13b | hr16: 62793514-62846866 | NSMUSG00000022911 | 8146 | .261557 | .0283403 | .23 |
| Ints6 | hr14: 63282503-63448963 | NSMUSG00000035161 | 8130 | .191376 | .0207465 | .22 |
| Plxna2 | hr1: 196446022-196643062 | NSMUSG00000026640 | 8845 | .13795 | .0150084 | .19 |
| Actr5, Mir3474 | hr2: 158450648-158464947 | A | A | .731723 | .0796205 | .19 |
| Yod1 | hr1: 132585619-132625830 | NSMUSG00000046404 | 26418 | .538936 | .059192 | .10 |
| Snora81 | hr16: 23107551-23127803 | NSMUSG00000087935 | A | 33.007 | 1.597 | .09 |
| Tssc1 | hr12: 29436692-29552356 | NSMUSG00000036613 | 80752 | .838137 | .0927738 | .03 |
| Gm684 | hr9: 51078362-51086659 | NSMUSG00000079559 | 00502940 | .5878 | .401094 | .95 |
| Sorl1 | hr9: 41776571-41932372 | NSMUSG00000049313 | 0660 | .10934 | .0122328 | .94 |
| Enpp4 | hr17: 44233258-44242757 | NSMUSG00000023961 | 24794 | .178242 | .0200871 | .87 |
| Anpep | hr7: 86966688-86987238 | NSMUSG00000039062 | 6790 | .254554 | .028699 | .87 |
| Mir126 | hr2: 26436575-26448202 | NSMUSG00000065540 | A | 305030 | 47297 | .86 |
| Snap23 | hr2: 120393406-120426458 | NSMUSG00000027287 | 0619 | .78909 | .203235 | .80 |
| Stap1 | hr5: 86500852-86533018 | NSMUSG00000029254 | 6792 | .858826 | .0976915 | .79 |
| Mir149 | hr1: 94728262-94756773 | NSMUSG00000065470 | A | 0759.9 | 229.42 | .75 |
| Tmtc4 | hr14: 123318196-123382483 | NSMUSG00000041594 | 0551 | .221309 | .0254519 | .70 |
| Snord66 | hr16: 20672821-20692956 | NSMUSG00000077239 | A | 661.06 | 88.672 | .62 |
| Efemp2 | hr19: 5474689-5481854 | NSMUSG00000024909 | 8859 | .31729 | .152871 | .62 |
| Snord68 | hr8: 125626249-125629142 | NSMUSG00000064450 | A | 9059.6 | 244.22 | .49 |
| Rsu1 | hr2: 12998635-13192905 | NSMUSG00000026727 | 0163 | .79585 | .213885 | .40 |
| Mtfmt | hr9: 65283588-65300861 | NSMUSG00000059183 | 9606 | .59961 | .309678 | .39 |
| A330035P11Rik | hr14: 122497160-122506196 | NSMUSG00000085615 | A | .36053 | .0430547 | .37 |
| Camsap1 | hr2: 25782357-25838802 | NSMUSG00000026933 | 27634 | .15705 | .618313 | .34 |
| Mapkap1 | hr2: 34287544-34480470 | NSMUSG00000038696 | 27743 | .4645 | .055846 | .32 |
| Abat | hr16: 8513521-8621660 | NSMUSG00000057880 | 68860 | .334455 | .0402357 | .31 |
| Plch2 | hr4: 154357223-154385093 | NSMUSG00000029055 | 69615 | .124503 | .0150096 | .29 |
| Kir3dl1 | hrX: 133052537-133068847 | NSMUSG00000031424 | 45616 | .452981 | .0547946 | .27 |
| Adcy3 | hr12: 4133396-4240123 | NSMUSG00000020654 | 04111 | .152245 | .0184347 | .26 |
| Kcnj16 | hr11: 110829346-110889281 | NSMUSG00000051497 | 6517 | .13349 | .500769 | .25 |
| Mir375 | hr1: 74947231-74947295 | NSMUSG00000065616 | A | 5654.5 | 897.99 | .25 |
| Ksr2 | hr5: 117864010-118217997 | NSMUSG00000061578 | 33050 | .237859 | .029018 | .20 |
| Aldh3a1 | hr11: 61022243-61031918 | NSMUSG00000019102 | 1670 | .00228 | .122463 | .18 |
| Cmpk2 | hr12: 27154079-27164702 | NSMUSG00000020638 | 2169 | .262415 | .0321326 | .17 |
| Fcnb | hr2: 27931998-27940398 | NSMUSG00000026835 | 4134 | .951222 | .116589 | .16 |
| Fam134b | hr15: 25773018-25903442 | NSMUSG00000022270 | 6270 | 3.8573 | .60438 | .15 |
| Hspa2 | hr12: 77505162-77507923 | NSMUSG00000059970 | 5512 | .721806 | .0888576 | .12 |
| Spink5 | hr18: 44122894-44182141 | NSMUSG00000055561 | 2432 | .391356 | .0485073 | .07 |
| Rnasek | hr11: 70051624-70053354 | NSMUSG00000040904 | 2898 | .80711 | .348209 | .06 |
| Evpl | hr11: 116081872-116099405 | NSMUSG00000034282 | 4027 | .0831717 | .0103221 | .06 |
| Gm16982 | hr7: 148748078-148818910 | A | A | .240089 | .029848 | .04 |
| Rpl39l | hr16: 10170320-10175004 | NSMUSG00000039209 | 8172 | .62387 | .203444 | .98 |
| Epb4.1l1 | hr2: 156246787-156368950 | NSMUSG00000027624 | 3821 | .124246 | .0155861 | .97 |
| 4930564K09Rik | hr3: 82680626-82747560 | NSMUSG00000086273 | A | .95865 | .120398 | .96 |
| Mrps24 | hr11: 5603985-5607702 | NSMUSG00000020477 | 4660 | .10473 | .518659 | .91 |
| Pdhb | hr14: 8998504-9005506 | NSMUSG00000021748 | 8263 | .581886 | .073944 | .87 |
| Cacna1c | hr6: 118542313-119171632 | NSMUSG00000051331 | 2288 | .0794284 | .0101186 | .85 |
| Krtap8-1 | hr16: 89487618-89488197 | NSMUSG00000059632 | 6703 | 642.87 | 37.471 | .83 |
| Myh14 | hr7: 51861172-51926213 | NSMUSG00000030739 | 1960 | .355738 | .0455464 | .81 |
| FR293140 | hr1: 139863030-139863130 | A | A | 8742.4 | 2643.3 | .81 |
| Paqr8 | hr1: 20880702-20928837 | NSMUSG00000025931 | 4229 | .400339 | .0512843 | .81 |
| Kirrel | hr3: 86882513-86978669 | NSMUSG00000041734 | 70643 | .190854 | .0246147 | .75 |
| Snap29 | hr16: 17280443-17430919 | NSMUSG00000022765 | 7474 | .01446 | .13102 | .74 |
| Rpl12 | hr2: 32817231-32819565 | NSMUSG00000038900 | 68706 | 4.645 | .76973 | .74 |
| Ntm | hr9: 28803548-29770714 | NSMUSG00000059974 | 35106 | .800421 | .103741 | .72 |
| 2010320M18Rik | hr8: 73300760-73301505 | A | A | .48825 | .841263 | .71 |
| Mir140 | hr8: 109960297-110082495 | NSMUSG00000065439 | A | 19393 | 19237 | .71 |
| Eml5 | hr12: 99985177-100139694 | NSMUSG00000051166 | 19670 | .0712626 | .00928874 | .67 |
| Snrpn, Snurf | hr7: 67127386-67285105 | A | A | .390515 | .0509414 | .67 |
| Cxcl2 | hr5: 91332924-91334964 | NSMUSG00000058427 | 0310 | 2.8431 | .68743 | .61 |
| Agbl5 | hr5: 31191224-31210161 | NSMUSG00000029165 | 31093 | .224777 | .0295419 | .61 |
| Svep1 | hr4: 58055667-58219468 | NSMUSG00000028369 | 4817 | .02525 | .398861 | .58 |
| A530046M15Rik | hr13: 15899075-15919370 | A | A | 9.6971 | .22574 | .55 |
| Tbck | hr3: 132347107-132501470 | NSMUSG00000028030 | 71981 | .23785 | .0317031 | .50 |
| Gvin1_dup1 | hr7: 113043632-113102484 | A | A | .0709466 | .00946535 | .50 |
| Atp4a | hr7: 31497250-31510553 | NSMUSG00000005553 | 1944 | .178115 | .0237707 | .49 |
| Limd2 | hr11: 106017569-106021456 | NSMUSG00000040699 | 32329 | .5537 | .341368 | .48 |
| Trim38 | hr13: 23874440-23883364 | NSMUSG00000064140 | 14158 | .530647 | .0713787 | .43 |
| Sirt6 | hr10: 81084530-81090353 | NSMUSG00000034748 | 0721 | .537895 | .0725839 | .41 |
| Lphn2 | hr3: 148478549-148617599 | NSMUSG00000028184 | 9633 | .46513 | .0629416 | .39 |
| Cpne4 | hr9: 104472117-104936874 | NSMUSG00000032564 | 4020 | .183636 | .0249962 | .35 |
| Rnf123 | hr9: 107951620-107981706 | NSMUSG00000041528 | 4585 | .15539 | .0212012 | .33 |
| Gja5 | hr3: 96836324-96857557 | NSMUSG00000057123 | 4613 | .412214 | .0562593 | .33 |
| FR268649 | hr2: 84581262-84581344 | A | A | 7838.1 | 0637.6 | .32 |
| Orc1 | hr4: 108252058-108287436 | NSMUSG00000028587 | 8392 | .666693 | .0917001 | .27 |
| Lif | hr11: 4157570-4172517 | NSMUSG00000034394 | 6878 | .91223 | .26308 | .27 |
| Chst5 | hr8: 114413034-114434099 | NSMUSG00000031952 | 6773 | .498378 | .068682 | .26 |
| AF357399 | hr7: 29135707-29137717 | A | A | 878.81 | 12.24 | .24 |
| FR246860, Mir125b-1 | hr9: 41389421-41400570 | A | A | 6925.3 | 902.57 | .20 |
| NA | hr1: 121549045-121551374 | A | A | .338797 | .0470486 | .20 |
| Ankrd49 | hr9: 14584641-14587408 | NSMUSG00000031931 | 6503 | .446825 | .0622621 | .18 |
| Tnfsf15 | hr4: 63385636-63406147 | NSMUSG00000050395 | 26623 | .673772 | .0938914 | .18 |
| Shroom3 | hr5: 93112460-93394785 | NSMUSG00000029381 | 7428 | .777824 | .108469 | .17 |
| Mgll | hr6: 88674405-88778354 | NSMUSG00000033174 | 3945 | .531573 | .0743003 | .15 |
| Hnrnpk | hr13: 58493311-58512510 | NSMUSG00000021546 | 5387 | .18484 | .726582 | .14 |
| Golph3l | hr3: 95392855-95423169 | NSMUSG00000046519 | 29593 | .311983 | .0438268 | .12 |
| Tgfb2 | hr1: 188447064-188529871 | NSMUSG00000039239 | 1808 | .174869 | .0247109 | .08 |
| Gm10677 | hr9: 47338434-47661468 | A | A | .322136 | .0455597 | .07 |
| Mlycd | hr8: 121918791-121934988 | NSMUSG00000074064 | 6690 | .350734 | .0501201 | .00 |
| Btrc | hr19: 45438223-45607833 | NSMUSG00000025217 | 2234 | .275533 | .0395115 | .97 |
| Ddit3 | hr10: 126727848-126748842 | NSMUSG00000025408 | 3198 | .27053 | .18222 | .97 |
| 1110018G07Rik | hr12: 86259098-86311836 | NSMUSG00000042350 | 8497 | .10931 | .0159043 | .87 |
| Sirt2 | hr7: 29551770-29573684 | NSMUSG00000015149 | 4383 | .85509 | .124596 | .86 |
| Dcstamp | hr15: 39577477-39592480 | NSMUSG00000022303 | 5766 | .317718 | .0464715 | .84 |
| Mtap1b | hr13: 100191418-100286557 | NSMUSG00000052727 | 7755 | .189799 | .027801 | .83 |
| Ugcg | hr4: 59202421-59235705 | NSMUSG00000028381 | 2234 | .720244 | .106088 | .79 |
| Alkbh8 | hr9: 3335230-3385846 | NSMUSG00000025899 | 7667 | .277536 | .0410234 | .77 |
| Scfd2 | hr5: 74600840-74927774 | NSMUSG00000062110 | 12986 | .180705 | .0267438 | .76 |
| Rps6kl1 | hr12: 86476545-86492214 | NSMUSG00000019235 | 38323 | .76887 | .114151 | .74 |
| Brcc3 | hrX: 72661966-72701040 | NSMUSG00000031201 | 10766 | .70356 | .253534 | .72 |
| Cacnb2 | hr2: 14525932-14909535 | NSMUSG00000057914 | 2296 | .387897 | .057937 | .70 |
| Tatdn1 | hr15: 58720783-58765285 | NSMUSG00000050891 | 9694 | 0.7267 | .60792 | .67 |
| Adad1 | hr3: 36962577-37010434 | NSMUSG00000027719 | 1744 | .244814 | .0368045 | .65 |
| Rccd1 | hr7: 87461501-87469340 | NSMUSG00000038930 | 69955 | .267436 | .0402393 | .65 |
| Irg1 | hr14: 103446228-103455790 | NSMUSG00000022126 | 6365 | 0.9213 | .64805 | .63 |
| Kcna4 | hr2: 107130745-107138661 | NSMUSG00000042604 | 6492 | .138999 | .021178 | .56 |
| Celsr1 | hr15: 85729187-85864207 | NSMUSG00000016028 | 2614 | .0518794 | .00792797 | .54 |
| Bves | hr10: 45055567-45089514 | NSMUSG00000071317 | 3828 | .877717 | .134211 | .54 |
| Phf14 | hr6: 11875880-12031198 | NSMUSG00000029629 | 5725 | .162402 | .0249967 | .50 |
| FR395873 | hr18: 61799306-61825192 | A | A | 0781.1 | 662.76 | .48 |
| NA | hr4: 128730224-128732351 | A | A | .673813 | .103969 | .48 |
| NA | hr5: 22928039-22931966 | A | A | .34825 | .0537348 | .48 |
| Zkscan4 | hr13: 21570717-21577374 | NSMUSG00000054931 | 44922 | .248751 | .0384413 | .47 |
| Adamts20 | hr15: 94100593-94234781 | NSMUSG00000022449 | 23838 | .0822738 | .0127289 | .46 |
| Eif3h | hr15: 51618108-51697007 | NSMUSG00000022312 | 8135 | 01.416 | 6.6596 | .46 |
| Stag2 | hrX: 39502588-39630352 | NSMUSG00000025862 | 0843 | .674124 | .104521 | .45 |
| Vac14 | hr8: 113142537-113244299 | NSMUSG00000010936 | 34729 | .40092 | .218021 | .43 |
| Krtap19-3 | hr16: 88877757-88878283 | A | A | .94908 | .148057 | .41 |
| Ndufb8 | hr19: 44624743-44629905 | NSMUSG00000025204 | 7264 | .53355 | .551334 | .41 |
| Cox16 | hr12: 82571690-82586097 | NSMUSG00000091803 | 6272 | .656819 | .102494 | .41 |
| Psma1 | hr7: 121408063-121419630 | NSMUSG00000030751 | 6440 | .07166 | .480871 | .39 |
| Rilpl1 | hr5: 124943088-124981400 | NSMUSG00000029392 | 5695 | .281453 | .044112 | .38 |
| FR232676 | hr17: 17967766-17967851 | A | A | 40490 | 00438 | .38 |
| Mir1940, Snora47 | hr13: 96095191-96107796 | A | A | 51.966 | 3.8593 | .37 |
| Tmsb4x | hrX: 163645025-163647150 | NSMUSG00000049775 | 9241 | 5.8728 | .20609 | .37 |
| Apob | hr12: 7984482-8023645 | NSMUSG00000020609 | 38055 | .0388061 | .00610331 | .36 |
| Gm20556 | hr15: 84545058-84602637 | A | A | .469451 | .0742743 | .32 |
| Lrp1b | hr2: 40452292-42509118 | NSMUSG00000049252 | 4217 | .0317439 | .00505811 | .28 |
| Rabl2 | hr15: 89412957-89422354 | NSMUSG00000022621 | 8708 | .353253 | .0565931 | .24 |
| Ccdc114 | hr7: 53183767-53204326 | NSMUSG00000040189 | 11535 | .17779 | .0285714 | .22 |
| Syt11 | hr3: 88548622-88576521 | NSMUSG00000068923 | 29521 | .254009 | .0408693 | .22 |
| Mccc1 | hr3: 35858230-35952469 | NSMUSG00000027709 | 2039 | .358406 | .0577456 | .21 |
| Cisd1 | hr10: 70793241-70807597 | NSMUSG00000037710 | 2637 | .755895 | .123362 | .13 |
| Zfp800 | hr6: 28189930-28211601 | NSMUSG00000039841 | 27049 | .183432 | .0300372 | .11 |
| Cdh4 | hr2: 179177182-179634080 | NSMUSG00000000305 | 2561 | .176265 | .0288805 | .10 |
| Tchhl1 | hr3: 93272675-93275902 | NSMUSG00000027908 | 1325 | .425342 | .0699302 | .08 |
| Gm9899 | hr5: 30876359-30927643 | NSMUSG00000053214 | A | .222993 | .0367411 | .07 |
| Zzef1 | hr11: 72609727-72740622 | NSMUSG00000055670 | 95018 | .403439 | .0666199 | .06 |
| Tppp | hr13: 74146866-74173201 | NSMUSG00000021573 | 2948 | .43354 | .402661 | .04 |
| Dennd1c | hr17: 57205477-57217933 | NSMUSG00000002668 | 0785 | .660378 | .110166 | .99 |
| Gsto2 | hr19: 47940034-47960795 | NSMUSG00000025069 | 8214 | .1945 | .199676 | .98 |
| Rsrc2 | hr5: 124178438-124199421 | NSMUSG00000029422 | 08606 | .666347 | .111997 | .95 |
| Mir148a | hr6: 51219810-51219909 | NSMUSG00000065505 | A | 559 | 610.34 | .94 |
| Deaf12l1 | hrX: 42139743-42143374 | NSMUSG00000045284 | 45404 | .857777 | .144604 | .93 |
| Actc1 | hr2: 113873024-113878547 | NSMUSG00000068614 | 1464 | .12595 | .18982 | .93 |
| Olfr1484 | hr19: 13659795-13660743 | NSMUSG00000096289 | 58288 | .94664 | .498889 | .91 |
| Gucy2e | hr11: 69031618-69050524 | NSMUSG00000020890 | 4919 | .126773 | .0215352 | .89 |
| Six3os1 | hr17: 86001271-86017736 | NSMUSG00000093460 | A | .187697 | .0320174 | .86 |
| Dbt | hr3: 116215996-116252899 | NSMUSG00000000340 | 3171 | .174741 | .029839 | .86 |
| Atp6v0e2 | hr6: 48487567-48491799 | NSMUSG00000039347 | 6252 | .801363 | .136892 | .85 |
| Mir20a | hr14: 115443378-115443485 | NSMUSG00000065442 | A | 0494.6 | 794.86 | .85 |
| Ccdc99 | hr11: 34622686-34647143 | NSMUSG00000069910 | 0385 | .386055 | .0662747 | .83 |
| Npl | hr1: 155350145-155396844 | NSMUSG00000042684 | 4091 | .430179 | .0739363 | .82 |
| Igbp1 | hrX: 97689629-97711464 | NSMUSG00000031221 | 8518 | .569352 | .097999 | .81 |
| Itpkc | hr7: 27992188-28013616 | NSMUSG00000003752 | 33011 | .32252 | .0556393 | .80 |
| Gdi1 | hrX: 71550350-71557206 | NSMUSG00000015291 | 4567 | .206732 | .0362875 | .70 |
| Aptx | hr4: 40629110-40650220 | NSMUSG00000028411 | 6408 | .0929809 | .0163342 | .69 |
| Epha7 | hr4: 28740294-28894649 | NSMUSG00000028289 | 3841 | .0891719 | .0157134 | .67 |
| Mir484 | hr16: 14159718-14159785 | NSMUSG00000070074 | A | 50894 | 4238.6 | .67 |
| Vps53 | hr11: 75859727-75993132 | NSMUSG00000017288 | 8299 | .494516 | .0872852 | .67 |
| Hcfc1r1 | hr17: 23797489-23814416 | NSMUSG00000023904 | 53502 | .566582 | .100028 | .66 |
| Myh6 | hr14: 55560757-55585444 | NSMUSG00000040752 | 7888 | .058954 | .0104266 | .65 |
| Duox1 | hr2: 122141407-122173708 | NSMUSG00000033268 | 9439 | .103087 | .018298 | .63 |
| Lpin3 | hr2: 160706405-160731736 | NSMUSG00000027412 | 4899 | .35034 | .239975 | .63 |
| Cdk5 | hr5: 23924059-23929348 | NSMUSG00000028969 | 2568 | .523815 | .0934182 | .61 |
| Gal3st2 | hr1: 95757920-95773071 | NSMUSG00000094651 | 81334 | .193791 | .0346162 | .60 |
| Nphp4 | hr4: 151852250-151937293 | NSMUSG00000039577 | 60305 | .36214 | .243862 | .59 |
| Casp3 | hr8: 47702802-47724062 | NSMUSG00000031628 | 2367 | .852557 | .152823 | .58 |
| Fam168a | hr7: 107855215-107990144 | NSMUSG00000029461 | 19604 | .802019 | .144028 | .57 |
| Corin | hr5: 72691263-72895779 | NSMUSG00000005220 | 3419 | .104196 | .0187281 | .56 |
| Prdx1 | hr4: 116357569-116372605 | NSMUSG00000028691 | 00862012 | .00519 | .360847 | .56 |
| Immp1l | hr2: 105744794-105805715 | NSMUSG00000042670 | 6541 | .50399 | .451204 | .55 |
| Tmem29 | hrX: 146832315-146893693 | NSMUSG00000090483 | 82245 | .666889 | .120441 | .54 |
| Kdr | hr5: 76329298-76374453 | NSMUSG00000062960 | 6542 | .182395 | .0329725 | .53 |
| 4931428F04Rik | hr8: 107800346-107813428 | NSMUSG00000014837 | 4356 | .141538 | .0256725 | .51 |
| FR005186 | hr16: 77599177-77599249 | A | A | 24701 | 5768.1 | .48 |
| 2210408I21Rik | hr13: 77274796-77752940 | NSMUSG00000071252 | 2371 | .0852413 | .0156019 | .46 |
| Hnrnpc | hr14: 52693054-52723703 | NSMUSG00000060373 | 5381 | .01105 | .185421 | .45 |
| Zdhhc16 | hr19: 42007961-42055626 | NSMUSG00000025157 | 4168 | .00977 | .18556 | .44 |
| Rfx3 | hr19: 27836210-28085656 | NSMUSG00000040929 | 9726 | .0536103 | .00988574 | .42 |
| Snx14 | hr9: 88271584-88333789 | NSMUSG00000032422 | 44962 | .450372 | .0831435 | .42 |
| NA | hr15: 12960407-12961346 | A | A | .93221 | .542926 | .40 |
| Pitpna | hr11: 75401609-75442280 | NSMUSG00000017781 | 8738 | .88756 | .351852 | .36 |
| Txn2 | hr15: 77745480-77759424 | NSMUSG00000005354 | 6551 | .819065 | .153466 | .34 |
| Olfr1477 | hr19: 13575035-13577782 | NSMUSG00000071629 | 58691 | .491256 | .092218 | .33 |
| 1500015O10Rik | hr1: 43787446-43799409 | NSMUSG00000026051 | 8896 | .734553 | .138167 | .32 |
| Lpin1 | hr12: 16542474-16596576 | NSMUSG00000020593 | 4245 | .309368 | .0586523 | .27 |
| Nhsl1 | hr10: 18127480-18253697 | NSMUSG00000039835 | 15819 | .353588 | .0670732 | .27 |
| Ccl4 | hr11: 83476085-83478185 | NSMUSG00000018930 | 0303 | 0.7929 | .86836 | .25 |
| Zcchc4 | hr5: 53174305-53211004 | NSMUSG00000029179 | 8796 | .18492 | .1813 | .24 |
| Dynlrb1 | hr2: 155062268-155076013 | NSMUSG00000047459 | 7068 | .34426 | .829834 | .24 |
| Gpt2 | hr8: 88016515-88051457 | NSMUSG00000031700 | 08682 | .237384 | .0454948 | .22 |
| Dnajc24 | hr2: 105806864-105843706 | NSMUSG00000027166 | 9349 | .43821 | .276134 | .21 |
| Commd9 | hr2: 101726418-101741796 | NSMUSG00000027163 | 6501 | .451462 | .0867977 | .20 |
| Pik3c2b | hr1: 134942588-135005265 | NSMUSG00000026447 | 40752 | .0641664 | .012369 | .19 |
| Snph | hr2: 151416285-151458269 | NSMUSG00000027457 | 41727 | .0990223 | .0191567 | .17 |
| Mir34a | hr4: 149442562-149442664 | NSMUSG00000065493 | A | 882.38 | 58.124 | .16 |
| Tulp4 | hr17: 6106829-6240637 | NSMUSG00000034377 | 8842 | .436195 | .0845033 | .16 |
| FR283132 | hr9: 50911136-50911216 | A | A | 47.479 | 7.1446 | .13 |
| Scube2 | hr7: 116942204-117009193 | NSMUSG00000007279 | 6788 | .700644 | .137069 | .11 |
| Xkr8 | hr4: 132280818-132288461 | NSMUSG00000037752 | 81560 | .470683 | .0920914 | .11 |
| Lrp4 | hr2: 91297687-91354058 | NSMUSG00000027253 | 28357 | .0529702 | .0103742 | .11 |
| Anks3 | hr16: 4941419-4964330 | NSMUSG00000022515 | 2615 | .145956 | .0286145 | .10 |
| Slc14a2 | hr18: 78342882-78793689 | NSMUSG00000024552 | 7411 | .0994956 | .0195607 | .09 |
| Meg3, Mir1906- | hr12: 110779205-110809939 | A | A | .233364 | .0460404 | .07 |
| 1, Mir770 | | | | | | |
| Krtap1-4 | hr11: 99443862-99444991 | NSMUSG00000075567 | 29873 | .926056 | .182752 | .07 |
| Pbk | hr14: 66424747-66436659 | NSMUSG00000022033 | 2033 | .54538 | .502744 | .06 |
| Gimap8 | hr6: 48597232-48610874 | NSMUSG00000064262 | 43374 | .122351 | .0241786 | .06 |
| Radil | hr5: 142960794-143027031 | NSMUSG00000029576 | 31858 | .03575 | .603146 | .03 |
| Mdga1 | hr17: 29964902-30024827 | NSMUSG00000043557 | 4762 | .0640198 | .0127582 | .02 |
| Hexdc | hr11: 121038906-121090623 | NSMUSG00000039307 | 38023 | .185937 | .0372847 | .99 |
| Xrcc6bp1 | hr10: 126305483-126338427 | NSMUSG00000025436 | 8876 | .3515 | .47267 | .97 |
| Fam189b | hr3: 88987146-88993211 | NSMUSG00000032657 | 8521 | .390645 | .0786906 | .96 |
| Acaa2 | hr18: 74938865-74965861 | NSMUSG00000036880 | 2538 | .20658 | .243316 | .96 |
| Iqcg | hr16: 32914185-33056272 | NSMUSG00000035578 | 9707 | .631126 | .12745 | .95 |
| Creb5 | hr6: 53523367-53645826 | NSMUSG00000053007 | 31991 | .132291 | .0267324 | .95 |
| Gm8909 | hr17: 36301388-36305482 | NSMUSG00000073402 | 67977 | .85036 | .576406 | .95 |
| Mir674 | hr2: 117010862-117010962 | NSMUSG00000076376 | A | 39.882 | 09.238 | .94 |
| Brsk2, Mir3104 | hr7: 149135655-149190148 | A | A | .5452 | .110568 | .93 |
| Mocs2 | hr13: 115608444-115619628 | NSMUSG00000015536 | 7434 | .725332 | .147283 | .92 |
| Shank1 | hr7: 51565633-51613723 | NSMUSG00000038738 | 43961 | .0498817 | .0101436 | .92 |
| Hspbp1 | hr7: 4612122-4636565 | NSMUSG00000063802 | 6245 | .56171 | .317805 | .91 |
| Pias2 | hr18: 77303946-77394447 | NSMUSG00000025423 | 7344 | .08722 | .221791 | .90 |
| Mrpl30 | hr1: 37947397-37955178 | NSMUSG00000026087 | 21542 | .96263 | .400638 | .90 |
| Xkrx | hrX: 130683583-130696467 | NSMUSG00000031258 | 31524 | .189979 | .0390272 | .87 |
| Akr1e1 | hr13: 4591735-4608410 | NSMUSG00000045410 | 6043 | .6378 | .131974 | .83 |
| Cntln | hr4: 84530230-84777822 | NSMUSG00000038070 | 38349 | .080951 | .0167645 | .83 |
| Mug1 | hr6: 121788558-121839075 | NSMUSG00000059908 | 7836 | .17266 | .0358445 | .82 |
| Mrps33 | hr6: 39751806-39760935 | NSMUSG00000029918 | 4548 | .14326 | .445898 | .81 |
| Fau | hr19: 6057887-6059524 | NSMUSG00000038274 | 4109 | .84198 | .175314 | .80 |
| Rps19-ps3 | hr4: 147195885-147196311 | NSMUSG00000080059 | A | .08178 | .05862 | .80 |
| Cmpk1 | hr4: 114633217-114659833 | NSMUSG00000028719 | 6588 | .99621 | .626134 | .79 |
| Atg14 | hr14: 48160567-48188109 | NSMUSG00000037526 | 00504663 | .573853 | .120137 | .78 |
| Greb1 | hr12: 16677420-16807692 | NSMUSG00000036523 | 68527 | .109647 | .0230816 | .75 |
| Ppp3ca | hr3: 136333733-136598743 | NSMUSG00000028161 | 9055 | .638753 | .134495 | .75 |
| 7-Sep | hr11: 53333237-53357598 | NSMUSG00000018398 | 0362 | .339851 | .0718838 | .73 |
| Rapsn | hr2: 90875783-90885886 | NSMUSG00000002104 | 9400 | .219063 | .0463783 | .72 |
| Sipa1l2 | hr8: 127941962-128016610 | NSMUSG00000001995 | 44668 | .542914 | .114975 | .72 |
| Synm | hr7: 74875046-74904628 | NSMUSG00000030554 | 33335 | .197959 | .042022 | .71 |
| Ccdc137 | hr11: 120319442-120328914 | NSMUSG00000049957 | 7291 | .24736 | .264856 | .71 |
| Reln | hr5: 21390271-21850523 | NSMUSG00000042453 | 9699 | .0349897 | .00744136 | .70 |
| Nol4 | hr18: 22851655-23200154 | NSMUSG00000041923 | 19211 | .0959601 | .0204135 | .70 |
| Gm5936 | hrX: 72082534-72088694 | NSMUSG00000079531 | 46325 | .138538 | .0295937 | .68 |
| Fut7 | hr2: 25279213-25281893 | NSMUSG00000036587 | 4347 | .230586 | .0493726 | .67 |
| Slc7a14 | hr3: 31101777-31209241 | NSMUSG00000069072 | 41919 | .0508887 | .0109136 | .66 |
| Mir350 | hr1: 178663783-178737255 | NSMUSG00000065573 | A | 412.11 | 161.12 | .66 |
| Zbtb20 | hr16: 43247396-43619236 | NSMUSG00000036279 | A | .265049 | .0568762 | .66 |
| Lime1 | hr2: 181115939-181118333 | NSMUSG00000090077 | 2699 | .255478 | .0548299 | .66 |
| Cenpo | hr12: 4133396-4240123 | NSMUSG00000020652 | 2504 | .541742 | .116317 | .66 |
| Gnpda2 | hr5: 69966240-69983524 | NSMUSG00000029209 | 7980 | .776939 | .167076 | .65 |
| Cdkl2 | hr5: 92435100-92472044 | NSMUSG00000029403 | 3886 | .245502 | .0531254 | .62 |
| Psmd8 | hr7: 29959205-29965692 | NSMUSG00000030591 | 7296 | .21151 | .262404 | .62 |
| Kcnb1 | hr2: 166921468-167014318 | NSMUSG00000050556 | 6500 | .0806768 | .0175079 | .61 |
| Vwf | hr6: 125502965-125636697 | NSMUSG00000001930 | A | .0401012 | .0087623 | .58 |
| Aasdh | hr5: 77304959-77334539 | NSMUSG00000055923 | 31326 | .115345 | .0253392 | .55 |
| Cacna2d3 | hr14: 29718128-30535050 | NSMUSG00000021991 | 2294 | .219859 | .0483318 | .55 |
| Al593442 | hr9: 52481146-52487534 | NSMUSG00000078307 | 30941 | .100341 | .0220949 | .54 |
| Fbxl17 | hr17: 63395300-63849929 | NSMUSG00000023965 | 0758 | .112549 | .0248087 | .54 |
| Dsc2 | hr18: 20189298-20218006 | NSMUSG00000024331 | 3506 | .87975 | .194402 | .53 |
| Vmn1r236 | hr17: 21423496-21424617 | NSMUSG00000054142 | 71235 | .14867 | .36558 | .50 |
| Zfp87 | hr13: 67616717-67627167 | A | A | .218398 | .0487703 | .48 |
| Rimkla | hr4: 119137890-119165203 | NSMUSG00000048899 | 94237 | .0999033 | .0223171 | .48 |
| Proser1 | hr3: 53267738-53285677 | A | A | .900434 | .202048 | .46 |
| Tm2d2 | hr8: 26127682-26133732 | NSMUSG00000031556 | 9742 | .62526 | .814656 | .45 |
| Gm16023 | hr4: 154975524-154998864 | NSMUSG00000086682 | A | .185697 | .0418352 | .44 |
| Slc6a18 | hr13: 73799197-73815471 | NSMUSG00000021612 | 2598 | .111782 | .0252995 | .42 |
| Kctd21 | hr7: 104480832-104498726 | NSMUSG00000044952 | 22320 | .684354 | .154925 | .42 |
| Xirp1 | hr9: 119922872-119932716 | NSMUSG00000079243 | 2437 | .457956 | .103717 | .42 |
| Zfp518b | hr5: 39059722-39076065 | NSMUSG00000046572 | 00515 | .0638599 | .0144756 | .41 |
| FR023578 | hr9: 108470641-108470732 | A | A | 12121 | 5416.3 | .41 |
| Mob2 | hr7: 149194457-149246939 | NSMUSG00000025147 | 01513 | .1664 | .492437 | .40 |
| Rragc | hr4: 123594675-123614240 | NSMUSG00000028646 | 4170 | .776282 | .176513 | .40 |
| Rbms3 | hr9: 116481863-117539031 | NSMUSG00000039607 | 07181 | .181473 | .0414025 | .38 |
| Foxn3 | hr12: 100433303-100688284 | NSMUSG00000033713 | 1375 | .318538 | .0729551 | .37 |
| Dopey2 | hr16: 93712151-93810833 | NSMUSG00000022946 | 0028 | .21755 | .049833 | .37 |
| Rsf1 | hr7: 104728405-104841292 | NSMUSG00000035623 | 33532 | .4203 | .554637 | .36 |
| Cby1 | hr15: 79489656-79498090 | NSMUSG00000022428 | 3739 | .51191 | .346472 | .36 |
| Dnaaf2 | hr12: 70290824-70300855 | A | A | .05851 | .242627 | .36 |
| Bean1 | hr8: 106694412-106742997 | NSMUSG00000031872 | 5115 | .140554 | .0322194 | .36 |
| Tigd2 | hr6: 59158863-59162027 | NSMUSG00000049232 | 8140 | .258914 | .0594159 | .36 |
| NA | hr6: 112559022-112560010 | A | A | .549902 | .127274 | .32 |
| NA | hr4: 94126714-94128934 | A | A | .214256 | .0495893 | .32 |
| NA | hr12: 110028197-110030901 | A | A | .172816 | .0399981 | .32 |
| NA | hr6: 99561279-99562928 | A | A | .298778 | .0691519 | .32 |
| NA | hr2: 157366082-157368080 | A | A | .240733 | .0557175 | .32 |
| NA | hr10: 94854787-94855840 | A | A | .507921 | .117558 | .32 |
| Kif1a | hr1: 94912032-94998442 | NSMUSG00000014602 | 6560 | .0415078 | .00961587 | .32 |
| Chl1 | hr6: 103460869-103683029 | NSMUSG00000030077 | 2661 | .0546958 | .0127171 | .30 |
| Mogat1 | hr1: 78507634-78534748 | NSMUSG00000012187 | 8393 | .28646 | .0669476 | .28 |
| Arhgap15 | hr2: 43604343-44243143 | NSMUSG00000049744 | 6117 | .159159 | .0372908 | .27 |
| BC096441, Tnfsf12, | hr11: 69496078-69509600 | A | A | .213399 | .0500087 | .27 |
| Tnfsf13 | | | | | | |
| Mpped2 | hr2: 106533615-106708517 | NSMUSG00000016386 | 7015 | .234618 | .0549958 | .27 |
| Nt5c2 | hr19: 46961320-47098836 | NSMUSG00000025041 | 6952 | .208941 | .0489975 | .26 |
| Fbxo41 | hr6: 85419571-85452880 | NSMUSG00000047013 | 30369 | .144635 | .0339905 | .26 |
| Cd101 | hr3: 100797451-100833418 | NSMUSG00000086564 | 30146 | .218311 | .0513536 | .25 |
| Tpt1 | hr14: 76245062-76248110 | NSMUSG00000060126 | 2070 | .95607 | .16743 | .25 |
| Lrif1 | hr3: 106487904-106539494 | A | A | .286496 | .0675203 | .24 |
| Fas | hr19: 34365148-34402260 | NSMUSG00000024778 | 4102 | .281842 | .0664719 | .24 |
| Alkbh2 | hr5: 114573942-114578185 | NSMUSG00000044339 | 31642 | .943495 | .223128 | .23 |
| Mir21, Vmp1 | hr11: 86397366-86497324 | A | A | 6926.8 | 1104.5 | .23 |
| Alb | hr5: 90889914-90905629 | NSMUSG00000029368 | 1657 | .192205 | .0455049 | .22 |
| Gsn | hr2: 35111878-35163422 | NSMUSG00000026879 | 27753 | .40673 | .333613 | .22 |
| Zfp703 | hr8: 28087807-28091934 | NSMUSG00000085795 | 53310 | .82497 | .433455 | .21 |
| Rbm22 | hr18: 60720439-60732383 | NSMUSG00000024604 | 6810 | .63332 | .863439 | .21 |
| Slc26a9 | hr1: 133640598-133666982 | NSMUSG00000042268 | 20718 | .0926796 | .0220394 | .21 |
| Acta1 | hr8: 126415666-126418636 | NSMUSG00000031972 | 1459 | .663091 | .157954 | .20 |
| Col4a5 | hrX: 137909961-138123778 | NSMUSG00000031274 | 2830 | .339378 | .081127 | .18 |
| Ulk2 | hr11: 61589099-61668594 | NSMUSG00000004798 | 9869 | .251791 | .0603681 | .17 |
| 4930523C07Rik | hr1: 161974510-162008717 | NSMUSG00000050883 | A | .100159 | .0240369 | .17 |
| Cldn14 | hr16: 93919275-94009082 | NSMUSG00000047109 | 6173 | .838541 | .201706 | .16 |
| Mmadhc | hr2: 50135400-50152197 | NSMUSG00000026766 | 09129 | .29149 | .551293 | .16 |
| Tiam2 | hr17: 3326572-3557713 | NSMUSG00000023800 | 4001 | .117719 | .0284395 | .14 |
| Clasrp | hr7: 20166391-20189817 | NSMUSG00000061028 | 3609 | .320523 | .0776906 | .13 |
| Mre11a | hr9: 14589150-14638861 | NSMUSG00000031928 | 7535 | .222113 | .0538471 | .12 |
| Fndc9 | hr11: 46007350-46126361 | A | A | .223227 | .0543385 | .11 |
| Glb1l3 | hr9: 26625537-26668408 | NSMUSG00000031966 | 0893 | .58207 | .386263 | .10 |
| Fgr | hr4: 132530009-132557797 | NSMUSG00000028874 | 4191 | .906271 | .22192 | .08 |
| Smchd1 | hr17: 71693833-71824683 | NSMUSG00000024054 | 4355 | .236098 | .0578371 | .08 |
| Adamts9 | hr6: 92722692-92851435 | NSMUSG00000030022 | 01401 | .53016 | .374882 | .08 |
| Homer1 | hr13: 94074449-94174917 | NSMUSG00000007617 | 6556 | .894864 | .219479 | .08 |
| Mir17hg, Mir19b-1 | hr14: 115443526-115445950 | A | A | 36312 | 3436.7 | .08 |
| Ndufa11 | hr17: 56857184-56863671 | NSMUSG00000002379 | 39760 | .293517 | .0721826 | .07 |
| FR003495, FR118095 | hrX: 18722641-18723660 | A | A | 0785.4 | 4986.1 | .06 |
| Pex16 | hr2: 92214832-92221377 | NSMUSG00000027222 | 8633 | .00107 | .246925 | .05 |
| Sh3glb1 | hr3: 144351807-144383287 | NSMUSG00000037062 | 4673 | .980593 | .242024 | .05 |
| Rims1 | hr1: 22278502-22812563 | NSMUSG00000041670 | A | .0893287 | .0220871 | .04 |
| Hdac2 | hr10: 36694349-36721694 | NSMUSG00000019777 | 5182 | .364108 | .0900844 | .04 |
| Fbn2 | hr18: 58168276-58369580 | NSMUSG00000024598 | 00047082 | .0331733 | .00822864 | .03 |
| Tm6sf1 | hr7: 89003907-89079345 | NSMUSG00000038623 | 07769 | .65555 | .410841 | .03 |
| 9930111J21Rik1, | hr11: 48759651-48792900 | A | A | .26395 | .065531 | .03 |
| 9930111J21Rik2_dup1 | | | | | | |
| Gm9125_dup1 | hr3: 93740554-93747465 | A | A | .402212 | .100113 | .02 |
| 2610028H24Rik | hr10: 75911825-75978602 | NSMUSG00000009114 | 6964 | .160739 | .0400206 | .02 |
| Morc2b | hr17: 33272534-33276628 | NSMUSG00000048602 | 40069 | .0978232 | .0243821 | .01 |
| Ddx47 | hr6: 134961629-134973794 | NSMUSG00000030204 | 7755 | .85909 | .464335 | .00 |
| Sh3d21 | hr4: 125827845-125840585 | A | A | .14033 | .285315 | .00 |
| Sec23a | hr12: 60059370-60113004 | NSMUSG00000020986 | 0334 | .284799 | .0713172 | .99 |
| Phf1 | hr17: 27070071-27074835 | NSMUSG00000024193 | 1652 | .181511 | .0455278 | .99 |
| Cyp11b1 | hr15: 74665321-74672073 | NSMUSG00000075604 | 10115 | .581064 | .146017 | .98 |
| Panx2 | hr15: 88890155-88901337 | NSMUSG00000058441 | 06218 | .28971 | .0728317 | .98 |
| Trpm3 | hr19: 22213606-23064374 | NSMUSG00000052387 | 26025 | .0964655 | .024259 | .98 |
| Pyhin1 | hr1: 175560989-175578059 | NSMUSG00000043263 | 36312 | .10256 | .0257922 | .98 |
| Zfp616 | hr11: 73883671-73900803 | NSMUSG00000069476 | 27963 | .441737 | .11114 | .97 |
| Rorb | hr19: 19005098-19185686 | NSMUSG00000036192 | 25998 | .876095 | .220742 | .97 |
| Acer3 | hr7: 105362169-105458037 | NSMUSG00000030760 | 6190 | .292969 | .0738793 | .97 |
| Adamtsl5 | hr10: 79803460-79811191 | NSMUSG00000043822 | 6548 | .29746 | .327388 | .96 |
| Stk30 | hr12: 112046007-112079149 | NSMUSG00000056458 | 6448 | .400888 | .101277 | .96 |
| Thap2 | hr10: 114807021-114821491 | NSMUSG00000020137 | 6816 | .169829 | .0429047 | .96 |
| Jakmip3 | hr7: 146132432-146269875 | NSMUSG00000056856 | 4004 | .124574 | .0315124 | .95 |
| Col4a6 | hrX: 137599945-137908619 | NSMUSG00000031273 | 4216 | .127462 | .0322523 | .95 |
| Zfp187 | hr13: 21534043-21545596 | NSMUSG00000022228 | 32731 | .1169 | .283878 | .93 |
| Acta2 | hr19: 34315580-34329826 | NSMUSG00000035783 | 1475 | .570206 | .144939 | .93 |
| Lilrb4 | hr10: 51210780-51216417 | NSMUSG00000062593 | 4728 | .35761 | .345727 | .93 |
| Slc1a6 | hr10: 78243240-78277570 | NSMUSG00000005357 | 0513 | .167882 | .042813 | .92 |
| Hunk | hr16: 90386641-90499798 | NSMUSG00000053414 | 6559 | .0785567 | .0201059 | .91 |
| Mir142 | hr11: 87570365-87570429 | NSMUSG00000065420 | A | 62674 | 1646.9 | .91 |
| Kdelr1 | hr7: 53128209-53139096 | NSMUSG00000002778 | 8137 | .346669 | .0888325 | .90 |
| Gpr114 | hr8: 97447593-97467190 | NSMUSG00000061577 | 82045 | .484464 | .124225 | .90 |
| Bbs2 | hr8: 96591853-96622711 | NSMUSG00000031755 | 7378 | .499874 | .128266 | .90 |
| Wdr83 | hr8: 87595655-87604645 | NSMUSG00000005150 | 7836 | .54311 | .396578 | .89 |
| Ptprf | hr4: 117880817-117964002 | NSMUSG00000033295 | 9268 | .224821 | .0579006 | .88 |
| Adamts1 | hr16: 85794072-85803360 | NSMUSG00000022893 | 1504 | .0653947 | .0168485 | .88 |
| Cxcl12 | hr6: 117118552-117131386 | NSMUSG00000061353 | 0315 | .227953 | .0587589 | .88 |
| Cxcl14 | hr13: 56390005-56397912 | NSMUSG00000021508 | 7266 | .202817 | .0522985 | .88 |
| Dnajc28 | hr16: 91614501-91619244 | NSMUSG00000039763 | 46738 | .117806 | .0303788 | .88 |
| FR509857 | hr11: 77886666-77886753 | A | A | 09104 | 9862 | .87 |
| Ccdc90a | hr13: 43633774-43655560 | NSMUSG00000021371 | 6137 | .104696 | .0270535 | .87 |
| Sh2d3c | hr2: 32576574-32610527 | NSMUSG00000059013 | 7387 | .276471 | .0715231 | .87 |
| Egfr | hr11: 16652205-16813910 | NSMUSG00000020122 | 3649 | .0659852 | .0170733 | .86 |
| Gnb2 | hr5: 137969356-137974457 | NSMUSG00000029713 | 4693 | .60404 | .70943 | .86 |
| Kcng4 | hr8: 122147753-122159580 | NSMUSG00000045246 | 6733 | .0881102 | .0228094 | .86 |
| Mir93 | hr5: 138605816-138613090 | NSMUSG00000065527 | A | 0171.7 | 634.99 | .86 |
| Olfr1179 | hr2: 88242165-88243089 | NSMUSG00000075127 | 58919 | .416679 | .108444 | .84 |
| Rhobtb3 | hr13: 76006984-76081272 | NSMUSG00000021589 | 3296 | .0760895 | .0198383 | .84 |
| Tie1 | hr4: 118143795-118162454 | NSMUSG00000033191 | 1846 | .38724 | .14479 | .83 |
| Ppl | hr16: 5086383-5132574 | NSMUSG00000039457 | 9041 | .163868 | .0427674 | .83 |
| Dspp | hr5: 104599730-104609146 | NSMUSG00000053268 | 66279 | .0653036 | .0170994 | .82 |
| Abtb2 | hr2: 103406466-103558580 | NSMUSG00000032724 | 9382 | .12704 | .295359 | .82 |
| Syngr4 | hr7: 53142214-53152081 | NSMUSG00000040231 | 8867 | .405067 | .106212 | .81 |
| Olfr435 | hr6: 43151644-43152586 | NSMUSG00000048693 | 58647 | .498835 | .130956 | .81 |
| Olfr137 | hr17: 38441465-38442404 | NSMUSG00000054940 | 58481 | .547426 | .143766 | .81 |
| Zscan22 | hr7: 13483163-13493588 | NSMUSG00000054715 | 32878 | .824026 | .216664 | .80 |
| Bcl3 | hr7: 20393810-20408104 | NSMUSG00000053175 | 2051 | .17765 | .310518 | .79 |
| Atp7b | hr8: 23104819-23170546 | NSMUSG00000006567 | 1979 | .0699484 | .0184497 | .79 |
| Ist1 | hr8: 112138416-112217194 | A | A | .78887 | .736607 | .79 |
| Ttc39c | hr18: 12802041-12895561 | NSMUSG00000024424 | 2747 | .342498 | .0905617 | .78 |
| Nlrc5 | hr8: 96996662-97051172 | NSMUSG00000074151 | A | .227218 | .0601733 | .78 |
| Hsf2bp | hr17: 32081713-32171453 | NSMUSG00000002076 | 4377 | .0613158 | .0162408 | .78 |
| Cox14 | hr15: 99556048-99558567 | A | A | .27937 | .605136 | .77 |
| Nf1 | hr11: 79153393-79395111 | NSMUSG00000020716 | 8015 | .32003 | .881617 | .77 |
| Mrvi1 | hr7: 118011780-118125609 | NSMUSG00000005611 | 7540 | .1748 | .0464422 | .76 |
| Ret | hr6: 118101765-118147762 | NSMUSG00000030110 | 9713 | .0554316 | .0147367 | .76 |
| Ndufb6 | hr4: 40217695-40226401 | NSMUSG00000071014 | 30075 | .81527 | .216862 | .76 |
| Ifih1 | hr2: 62433849-62484312 | NSMUSG00000026896 | 1586 | .480321 | .127808 | .76 |
| Vmn1r62 | hr7: 5626205-5628199 | NSMUSG00000060420 | A | .217044 | .0578871 | .75 |
| Tpk1 | hr6: 43295005-43616174 | NSMUSG00000029735 | 9807 | .370925 | .0992681 | .74 |
| Znhit1 | hr5: 137458070-137472516 | NSMUSG00000059518 | 0103 | .428872 | .114782 | .74 |
| Smyd2 | hr1: 191704370-191746167 | NSMUSG00000026603 | 26830 | .259406 | .0694421 | .74 |
| C030006K11Rik, Lrrc24 | hr15: 76545705-76554275 | A | A | 9.6199 | .93398 | .73 |
| Oas2 | hr5: 121180341-121199857 | NSMUSG00000032690 | 46728 | .694886 | .186325 | .73 |
| Alpk3 | hr7: 88202485-88250498 | NSMUSG00000038763 | 16904 | .47932 | .20412 | .72 |
| Rag1 | hr2: 101478410-101489689 | NSMUSG00000061311 | 9373 | .393704 | .105907 | .72 |
| Trafd1 | hr5: 121821733-121835624 | NSMUSG00000042726 | 31712 | .41157 | .380087 | .71 |
| Pitpnm3 | hr11: 71861029-71949391 | NSMUSG00000040543 | 27958 | .0484678 | .0130544 | .71 |
| Gltscr1 | hr7: 16556610-16584844 | NSMUSG00000070808 | 43842 | .235183 | .0633541 | .71 |
| Cr1l | hr1: 196929981-196957764 | NSMUSG00000016481 | 2946 | .39537 | .379368 | .68 |
| Siglecg | hr7: 50663649-50673719 | NSMUSG00000030468 | 43958 | .160159 | .043544 | .68 |
| Dctd | hr8: 49184445-49227021 | NSMUSG00000031562 | 20685 | .588028 | .160205 | .67 |
| Fuca1 | hr4: 135476640-135496215 | NSMUSG00000028673 | 1665 | .4838 | .404627 | .67 |
| Mirlet7b | hr15: 85537748-85537833 | NSMUSG00000065564 | A | 368.67 | 196.13 | .65 |
| Ap5s1 | hr2: 131036175-131039250 | A | A | .327946 | .0898263 | .65 |
| Med23 | hr10: 24589791-24633335 | NSMUSG00000019984 | 0208 | .384081 | .105362 | .65 |
| Myog | hr1: 136186580-136189125 | NSMUSG00000026459 | 7928 | .23288 | .0639618 | .64 |
| Psma4 | hr9: 54798063-54805837 | NSMUSG00000032301 | 6441 | .16114 | .318962 | .64 |
| Grk1 | hr8: 13405080-13421949 | NSMUSG00000031450 | 4013 | .520177 | .14323 | .63 |
| Aldoa | hr7: 133938747-133943961 | NSMUSG00000030695 | 1674 | 5.023 | .13837 | .63 |
| Efna5 | hr17: 62952305-63230666 | NSMUSG00000090425 | A | .0788611 | .0217315 | .63 |
| Slc2a10 | hr2: 165329477-165345411 | NSMUSG00000027661 | 70441 | .119571 | .0329675 | .63 |
| Zfp704 | hr3: 9427009-9610085 | NSMUSG00000040209 | 70753 | .0250452 | .00690889 | .63 |
| Cnn2 | hr10: 79451344-79458145 | NSMUSG00000004665 | 2798 | .154567 | .0426449 | .62 |
| Mir574 | hr5: 65361313-65433140 | NSMUSG00000077042 | A | 8677.7 | 917.45 | .62 |
| Mir24-2 | hr8: 86732713-86732820 | NSMUSG00000065541 | A | 1337.1 | 4180 | .62 |
| Parp6 | hr9: 59465090-59498097 | NSMUSG00000025237 | 7287 | .115012 | .0318063 | .62 |
| Zfp493 | hr13: 67880629-67890017 | NSMUSG00000090659 | 2958 | .147346 | .040749 | .62 |
| Zfp560 | hr9: 20149579-20189602 | NSMUSG00000045519 | 34377 | .46683 | .40591 | .61 |
| Tln2 | hr9: 67064891-67407510 | NSMUSG00000052698 | 0549 | .164476 | .0455837 | .61 |
| Nudt1 | hr5: 140807875-140814089 | NSMUSG00000036639 | 7766 | .36019 | .377102 | .61 |
| Olfr235 | hr19: 12342721-12343660 | NSMUSG00000060049 | 58681 | .5065 | .417731 | .61 |
| Scn1b | hr7: 31901542-31911964 | NSMUSG00000019194 | 0266 | .281056 | .0780418 | .60 |
| NA | hr9: 67424977-67428226 | A | A | .236516 | .0656898 | .60 |
| NA | hr13: 98280390-98283149 | A | A | .281832 | .0782759 | .60 |
| Dock9 | hr14: 121941260-122196956 | NSMUSG00000025558 | 05445 | .166892 | .0463937 | .60 |
| Fam82a1 | hr17: 80014239-80081492 | NSMUSG00000036368 | 81110 | .194566 | .054139 | .59 |
| Slc4a10 | hr2: 61884571-62164800 | NSMUSG00000026904 | 4229 | .0216051 | .00601332 | .59 |
| Xrn1 | hr9: 95855178-95953448 | NSMUSG00000032410 | 4127 | .401311 | .112066 | .58 |
| Ttc23l | hr15: 10433701-10488423 | NSMUSG00000022249 | 5777 | .164625 | .0460494 | .57 |
| Dbpht2 | hr12: 75398460-75401455 | NSMUSG00000029878 | 86753 | .121747 | .0341126 | .57 |
| Prkaa2 | hr4: 104702254-104782503 | NSMUSG00000028518 | 08079 | .141522 | .0397649 | .56 |
| Card14 | hr11: 119176100-119216824 | NSMUSG00000013483 | 70720 | .0633294 | .0178709 | .54 |
| Rbm42 | hr7: 31426013-31435247 | NSMUSG00000036733 | 8035 | .33394 | .658712 | .54 |
| Tmem72 | hr6: 116642742-116666776 | NSMUSG00000048108 | 19776 | .100918 | .0285055 | .54 |
| Kif18a | hr2: 109120894-109181903 | NSMUSG00000027115 | 28421 | .516844 | .146053 | .54 |
| Idh2 | hr7: 87239733-87260236 | NSMUSG00000030541 | 69951 | .55729 | .157565 | .54 |
| Wdr44 | hrX: 23270242-23383127 | NSMUSG00000036769 | 2404 | .0849226 | .0240368 | .53 |
| Trim46 | hr3: 89038098-89049819 | NSMUSG00000042766 | 60213 | 7.1905 | 6.2132 | .53 |
| Osbpl6 | hr2: 76244594-76438704 | NSMUSG00000042359 | 9031 | .131265 | .0372232 | .53 |
| Tmem63c | hr12: 88367513-88430989 | NSMUSG00000034145 | 17733 | .0864327 | .0245126 | .53 |
| Zhx3 | hr2: 160557045-160698726 | NSMUSG00000035877 | 20799 | .513774 | .146146 | .52 |
| Ncf2 | hr1: 154655019-154684120 | NSMUSG00000026480 | 7970 | .09391 | .597517 | .50 |
| 4933433H22Rik | hr17: 84477999-84489215 | A | A | .11705 | .31632 | .50 |
| Enpp6 | hr8: 48072278-48180249 | NSMUSG00000038173 | 20981 | .291747 | .083326 | .50 |
| Gm11437 | hr11: 83961864-83980978 | NSMUSG00000051452 | 28813 | .199834 | .0573188 | .49 |
| Csf3 | hr11: 98562626-98564943 | NSMUSG00000038067 | 2985 | 3.0714 | .74948 | .49 |
| Snta1 | hr2: 154202049-154233820 | NSMUSG00000027488 | 0648 | .45904 | .41909 | .48 |
| Nbeal1 | hr1: 60237442-60391549 | NSMUSG00000073664 | 69198 | .872872 | .251792 | .47 |
| Cep192 | hr18: 67959760-68044824 | NSMUSG00000024542 | 0799 | .486627 | .140952 | .45 |
| Syngr2 | hr11: 117670980-117675600 | NSMUSG00000048277 | 0973 | .19901 | .638586 | .44 |
| Nod2 | hr8: 91171245-91212373 | NSMUSG00000055994 | 57632 | .17503 | .341393 | .44 |
| Dclk1 | hr3: 55046447-55342990 | NSMUSG00000027797 | 3175 | .0545099 | .0158508 | .44 |
| Mir92-2 | hrX: 50095014-50095105 | NSMUSG00000065613 | A | 262.03 | 533.06 | .43 |
| Grm5 | hr7: 94732677-95283573 | NSMUSG00000049583 | 08071 | .0791133 | .0230521 | .43 |
| Ncam2 | hr16: 81200941-81624532 | NSMUSG00000022762 | 7968 | .0933267 | .0272112 | .43 |
| Nyap2 | hr1: 81073891-81268226 | A | A | .0627261 | .0182898 | .43 |
| Trappc11 | hr8: 48575481-48618824 | A | A | .122121 | .0356517 | .43 |
| Opn1mw | hrX: 71372804-71396094 | NSMUSG00000031394 | 4539 | .316724 | .0928279 | .41 |
| Kcnma1 | hr14: 24117915-24823427 | NSMUSG00000063142 | 6531 | .352927 | .103439 | .41 |
| Mir181b-1 | hr1: 139863215-139863295 | NSMUSG00000065458 | A | 0311.6 | 024.74 | .41 |
| Pspc1 | hr14: 57341285-57397153 | NSMUSG00000021938 | 6645 | .268427 | .0787608 | .41 |
| Arid5a | hr1: 36364577-36380874 | NSMUSG00000037447 | 14855 | .393599 | .115777 | .40 |
| FR128586 | hrX: 18724007-18724110 | A | A | 5246.6 | 3314.5 | .40 |
| Nbea | hr3: 55429119-55987623 | NSMUSG00000027799 | 6422 | .135447 | .0398641 | .40 |
| Olfr566 | hr7: 110004834-110005785 | NSMUSG00000060888 | 58168 | .449408 | .132313 | .40 |
| Gzmn | hr14: 56784632-56793433 | NSMUSG00000015443 | 45839 | .87263 | .257185 | .39 |
| Socs3 | hr11: 117827400-117830680 | NSMUSG00000053113 | 2702 | 1.4574 | 8.1255 | .39 |
| Mrto4 | hr4: 138903359-138908213 | NSMUSG00000028741 | 68455 | .22271 | .656815 | .38 |
| L1td1 | hr4: 98393444-98405177 | NSMUSG00000087166 | 81591 | .0972611 | .0287746 | .38 |
| Irs1 | hr1: 82229679-82288014 | NSMUSG00000055980 | 6367 | .487459 | .144567 | .37 |
| Vti1b | hr12: 80231774-80273445 | NSMUSG00000021124 | 3612 | .579172 | .171783 | .37 |
| Zfp768 | hr7: 134486308-134488828 | NSMUSG00000047371 | 33890 | .28858 | .382202 | .37 |
| Lrrc8c | hr5: 105948489-106037973 | NSMUSG00000054720 | 00604 | .0870303 | .025825 | .37 |
| Sec31b | hr19: 44591446-44620338 | NSMUSG00000051984 | 40667 | .0668836 | .019926 | .36 |
| 2700038G22Rik | hr5: 23356414-23360851 | A | A | .89441 | .56445 | .36 |
| Dexi | hr16: 10530299-10543147 | NSMUSG00000038055 | 8239 | .259966 | .0775823 | .35 |
| Mxd1 | hr6: 86597038-86619153 | NSMUSG00000001156 | 7119 | .509474 | .152378 | .34 |
| Anxa4 | hr6: 86686833-86743578 | NSMUSG00000029994 | 1746 | .371276 | .111063 | .34 |
| Mir17 | hr14: 115442892-115442976 | NSMUSG00000065508 | A | 6089.2 | 8799.2 | .34 |
| Cds1 | hr5: 102194148-102252871 | NSMUSG00000029330 | 4596 | .0822506 | .0246951 | .33 |
| Bhlha15 | hr5: 144951154-144955310 | NSMUSG00000052271 | 7341 | .0846741 | .0255105 | .32 |
| Snord8 | hr14: 52817825-52857247 | NSMUSG00000093044 | A | 42.453 | 23.984 | .31 |
| Fam54a | hr10: 20067624-20081475 | NSMUSG00000019992 | 1804 | .154202 | .0465647 | .31 |
| Chrng | hr1: 89102385-89108410 | NSMUSG00000026253 | 1449 | .25652 | .0774836 | .31 |
| H2-Eb1 | hr17: 34442811-34453619 | NSMUSG00000060586 | 4969 | .677582 | .204671 | .31 |
| Sdc3 | hr4: 130348451-130382233 | NSMUSG00000025743 | 0970 | .202447 | .0611526 | .31 |
| Snord42b | hr11: 77994436-77997086 | NSMUSG00000065676 | A | 8789.2 | 678.5 | .31 |
| Hspbap1 | hr16: 35770471-35828548 | NSMUSG00000022849 | 6667 | .0870555 | .0263101 | .31 |
| Anxa6 | hr11: 54792463-54846973 | NSMUSG00000018340 | 1749 | .760841 | .230176 | .31 |
| Nipsnap3b | hr4: 53024795-53034931 | NSMUSG00000015247 | 6536 | .813704 | .246468 | .30 |
| Irgm1 | hr11: 48678750-48684848 | NSMUSG00000046879 | 5944 | .55806 | .47204 | .30 |
| Ino80e | hr7: 133991066-134004977 | NSMUSG00000030689 | 33875 | .40451 | .0332 | .30 |
| Tox3 | hr8: 92771010-92872151 | NSMUSG00000043668 | 44579 | .287201 | .0872433 | .29 |
| Slc10a5 | hr3: 10331733-10335656 | NSMUSG00000058921 | 41877 | .52421 | .463445 | .29 |
| Mcin | hr13: 113784075-113790602 | A | A | .243953 | .0741931 | .29 |
| Gm8994 | hr6: 136277556-136280001 | NSMUSG00000094973 | 68137 | .124189 | .0377703 | .29 |
| Fadd | hr7: 151764227-151768341 | NSMUSG00000031077 | 4082 | .278213 | .0847374 | .28 |
| Hint1 | hr11: 54679939-54683998 | NSMUSG00000020267 | 5254 | .20151 | .97687 | .28 |
| Arl14ep | hr2: 106802685-106814554 | A | A | .322935 | .098606 | .28 |
| Frem3 | hr8: 83134937-83219456 | NSMUSG00000042353 | 33315 | .11693 | .0357227 | .27 |
| 5430428K19Rik | hrX: 5977262-6210835 | A | A | .414061 | .126583 | .27 |
| Cdk2ap2 | hr19: 4097350-4099017 | NSMUSG00000024856 | 2004 | .09537 | .335247 | .27 |
| Vps16 | hr2: 130243419-130270005 | NSMUSG00000027411 | 0743 | .590208 | .180687 | .27 |
| Ucp2 | hr7: 107641853-107650682 | NSMUSG00000033685 | 2228 | .03722 | .317679 | .26 |
| Mll3 | hr5: 24777611-25004601 | NSMUSG00000038056 | 31051 | .0746061 | .0228618 | .26 |
| Cacna2d1 | hr5: 15440508-15880329 | NSMUSG00000040118 | 2293 | .0464064 | .0142787 | .25 |
| Arhgef26 | hr3: 62142698-62266143 | NSMUSG00000036885 | 22434 | .0554297 | .0170666 | .25 |
| Mpst | hr15: 78237141-78244445 | NSMUSG00000071711 | 46221 | .303278 | .0935586 | .24 |
| L2hgdh | hr12: 70791422-70825861 | NSMUSG00000020988 | 17666 | .243142 | .0750089 | .24 |
| Parp14 | hr16: 35832963-35871468 | NSMUSG00000034422 | 47253 | .22015 | .684976 | .24 |
| Numbl | hr7: 28043779-28067169 | NSMUSG00000063160 | 8223 | .110908 | .0342207 | .24 |
| Tbl1x | hrX: 74756565-74905604 | NSMUSG00000025246 | 1372 | .688383 | .213107 | .23 |
| Gm6307 | hr2: 180105562-180136507 | A | A | 1.4602 | 2.8401 | .23 |
| Phlda1 | hr10: 110943341-110945705 | NSMUSG00000020205 | 1664 | .9524 | .605051 | .23 |
| Mgarp | hr3: 51192334-51200469 | A | A | .272196 | .0843932 | .23 |
| Rbfa | hr18: 80389002-80397358 | NSMUSG00000024570 | 8731 | .722247 | .22403 | .22 |
| Dchs1 | hr7: 112901502-112936064 | NSMUSG00000036862 | 33651 | .0246675 | .0076542 | .22 |
| Zfp52 | hr17: 21672502-21699565 | NSMUSG00000051341 | 2710 | .918618 | .285113 | .22 |
| Nln | hr13: 104813518-104899694 | NSMUSG00000021710 | 5805 | .349817 | .108674 | .22 |
| Cnga2 | hrX: 69237213-69255557 | NSMUSG00000005864 | 2789 | .194019 | .0604208 | .21 |
| Dner | hr1: 84366413-84692796 | NSMUSG00000036766 | 27325 | .465354 | .145082 | .21 |
| Gvin1_dup2 | hr7: 113300049-113358854 | A | A | .0607026 | .018941 | .20 |
| Atp6v1e1 | hr6: 120745261-120772703 | NSMUSG00000019210 | 1973 | .3535 | .422767 | .20 |
| Cnnm4 | hr1: 36528441-36565621 | NSMUSG00000037408 | 4220 | .180585 | .0564097 | .20 |
| Mob1b | hr5: 89149895-89187480 | NSMUSG00000006262 | 8473 | .69899 | .531014 | .20 |
| Cwc22 | hr2: 77733709-77784410 | NSMUSG00000027014 | 0744 | .36702 | .42752 | .20 |
| Leap2 | hr11: 53235682-53236638 | NSMUSG00000036216 | 59301 | .02423 | .320485 | .20 |
| Usp43 | hr11: 67668024-67735655 | NSMUSG00000020905 | 16835 | .0617156 | .0193786 | .18 |
| Slit2 | hr5: 48374393-48697017 | NSMUSG00000031558 | 0563 | .154376 | .0484953 | .18 |
| Rpgr | hrX: 9735341-9793921 | NSMUSG00000031174 | 9893 | .319193 | .100296 | .18 |
| Psma8 | hr18: 14864659-14920808 | NSMUSG00000036743 | 3677 | .191478 | .0601724 | .18 |
| Ano2 | hr6: 125640436-125990146 | NSMUSG00000038115 | 43634 | .0673501 | .0211785 | .18 |
| Itgal | hr7: 134439773-134478651 | NSMUSG00000030830 | 6408 | .055757 | .0175625 | .17 |
| Mir30c-2 | hr1: 23298539-23298623 | NSMUSG00000065567 | A | 7223 | 8034.7 | .17 |
| Klra1, Klra12, Klra13- | hr6: 129922099-130336892 | A | A | .197891 | .0624405 | .17 |
| ps, Klra15, Klra18, Klra22, | | | | | | |
| Klra23, Klra33, Klra4 | | | | | | |
| NA | hr10: 29418799-29419163 | A | A | .7352 | .07255 | .17 |
| Epha3 | hr16: 63545043-63863983 | NSMUSG00000052504 | 3837 | .347324 | .109889 | .16 |
| Ring1 | hr17: 34157736-34161625 | NSMUSG00000024325 | 9763 | .59633 | .505412 | .16 |
| Tdpoz2 | hr3: 93455463-93456608 | A | A | .49358 | .156408 | .16 |
| Tecta | hr9: 42137704-42208012 | NSMUSG00000037705 | 1683 | .0366675 | .0116637 | .14 |
| 4933417G07Rik | hr3: 55429119-55987623 | A | A | .25302 | .398849 | .14 |
| Plxdc2 | hr2: 16277948-16673742 | NSMUSG00000026748 | 7448 | .178495 | .0568317 | .14 |
| Nat8l | hr5: 34338632-34348565 | NSMUSG00000048142 | 69642 | .10387 | .033072 | .14 |
| St6gal2 | hr17: 55585014-55638524 | NSMUSG00000024172 | 40119 | .13233 | .0421668 | .14 |
| Cdh22 | hr2: 164937006-165060237 | NSMUSG00000053166 | 04010 | .0732188 | .0233484 | .14 |
| Sf3b5 | hr10: 12728255-12728989 | NSMUSG00000078348 | 6125 | .74876 | .558913 | .13 |
| Wdtc1 | hr4: 132848380-132895230 | NSMUSG00000037622 | 30796 | .143805 | .0459687 | .13 |
| AF529169 | hr9: 89484871-89517824 | NSMUSG00000039313 | 09743 | .0574352 | .0183957 | .12 |
| Cpz | hr5: 35844866-35868275 | NSMUSG00000036596 | 42939 | .122089 | .0391317 | .12 |
| Dbndd1 | hr8: 126028617-126039355 | NSMUSG00000031970 | 2185 | .475002 | .152274 | .12 |
| Spag5 | hr11: 78115092-78135956 | NSMUSG00000002055 | 4141 | .739591 | .237361 | .12 |
| Sccpdh | hr1: 181598361-181617315 | NSMUSG00000038936 | 09232 | .543322 | .174402 | .12 |
| Lrrc4b | hr7: 51697856-51718714 | NSMUSG00000047085 | 72381 | .110522 | .0355391 | .11 |
| Cyp4x1 | hr4: 114781286-114806582 | NSMUSG00000047155 | 1906 | .196772 | .0632954 | .11 |
| Fut9 | hr4: 25536479-25727150 | NSMUSG00000055373 | 4348 | .730617 | .235226 | .11 |
| Ank1 | hr8: 24085353-24260968 | NSMUSG00000031543 | 1733 | .142829 | .046041 | .10 |
| Ces1e | hr8: 95725116-95753518 | NSMUSG00000061959 | 3897 | .470949 | .151981 | .10 |
| Arpc2 | hr1: 74283123-74314787 | NSMUSG00000006304 | 6709 | .56294 | .47393 | .10 |
| Zfp81 | hr17: 33470672-33495823 | NSMUSG00000003929 | 24694 | .254728 | .0824639 | .09 |
| Inadl | hr4: 98062516-98386294 | NSMUSG00000061859 | 2695 | .10102 | .0327153 | .09 |
| Gm14634 | hrX: 12339403-12398618 | NSMUSG00000085891 | A | .219733 | .0713813 | .08 |
| Tmem63b | hr17: 45797125-45823167 | NSMUSG00000036026 | 24807 | .104649 | .0340824 | .07 |
| Slit1 | hr19: 41674746-41818346 | NSMUSG00000025020 | 0562 | .0493344 | .0160898 | .07 |
| Scnn1g | hr7: 128878020-128911991 | NSMUSG00000000216 | 0278 | .0926076 | .0302049 | .07 |
| Tnmd | hrX: 130385546-130400116 | NSMUSG00000031250 | 4103 | .617603 | .201547 | .06 |
| Arhgap32 | hr9: 31923720-32073096 | NSMUSG00000041444 | 30914 | .0552916 | .0180741 | .06 |
| Mbd1 | hr18: 74427941-74442338 | NSMUSG00000024561 | 7190 | .58835 | .519257 | .06 |
| Add2 | hr6: 85978674-86074403 | NSMUSG00000030000 | 1519 | .171156 | .0559761 | .06 |
| Ccdc85c | hr12: 109444554-109513627 | NSMUSG00000084883 | 68158 | .918332 | .300761 | .05 |
| Tet1 | hr10: 62267274-62342762 | NSMUSG00000047146 | 2463 | .0228872 | .00749596 | .05 |
| C030037D09Rik | hr11: 88579956-88589886 | NSMUSG00000087574 | A | .528213 | .17304 | .05 |
| Adcy1 | hr11: 6963491-7078508 | NSMUSG00000020431 | 32530 | .316352 | .103923 | .04 |
| Zbtb24 | hr10: 41170201-41185380 | NSMUSG00000019826 | 68294 | .678334 | .222974 | .04 |
| Stab1 | hr14: 31948038-31981827 | NSMUSG00000042286 | 92187 | .0265267 | .00873585 | .04 |
| Xkr6 | hr14: 64225366-64439247 | NSMUSG00000035067 | 19149 | .10934 | .036119 | .03 |
| Pla2g4b | hr2: 119859168-119868768 | NSMUSG00000033852 | 33466 | .0641633 | .0212074 | .03 |
| Dlst | hr12: 86451782-86475041 | NSMUSG00000004789 | 8920 | 5.7274 | 8.4403 | .02 |
| 4932413F04Rik | hr9: 103385265-103664167 | A | A | .12144 | .702835 | .02 |
| Siglec1 | hr2: 130894955-130912501 | NSMUSG00000027322 | 0612 | .71477 | .901917 | .01 |
| Gm13103 | hr4: 143436399-143443540 | NSMUSG00000029451 | 94225 | 5.6431 | .21179 | .00 |
| Pcsk9 | hr4: 106114938-106136930 | NSMUSG00000044254 | 00102 | .0814567 | .027142 | .00 |
| Nsfl1c | hr2: 151320043-151337040 | NSMUSG00000027455 | 86649 | .8752 | .291627 | .00 |
| Zswim2 | hr2: 83755235-83781383 | NSMUSG00000034552 | 1861 | .136863 | .0456093 | .00 |
| Mirlet7g | hr9: 106073259-106094037 | NSMUSG00000065440 | A | 2806.9 | 0930.4 | .00 |
| Actn2 | hr13: 12361693-12432999 | NSMUSG00000052374 | 1472 | .0566392 | .018876 | .00 |
| Cox5b | hr1: 36748331-36750233 | NSMUSG00000061518 | 00046079 | .6987 | .90043 | .00 |
| Tbr1 | hr2: 61642509-61652170 | NSMUSG00000035033 | 1375 | .0866845 | .0289288 | .00 |
| Nr4a1 | hr15: 101097276-101105225 | NSMUSG00000023034 | 5370 | .34938 | .450454 | .00 |
| Rslcan18, Zfp708 | hr13: 67170333-67214964 | A | A | .165638 | .0554789 | .99 |
| Clec4e | hr6: 123231806-123239889 | NSMUSG00000030142 | 6619 | .518729 | .173764 | .99 |
| Rcn3 | hr7: 52338283-52347583 | NSMUSG00000019539 | 2377 | .172676 | .0579115 | .98 |
| Dennd5b | hr6: 148936590-149050202 | NSMUSG00000030313 | 20560 | .242102 | .0811984 | .98 |
| 1700084F23Rik | hr13: 70142927-70167226 | A | A | .209272 | .0702687 | .98 |
| Ifltd1 | hr6: 145345754-145383445 | NSMUSG00000054966 | 4071 | .203071 | .068364 | .97 |
| Zfp692 | hr11: 58120570-58128115 | NSMUSG00000037243 | 03836 | .40377 | .472705 | .97 |
| Wdr4 | hr17: 31631266-31649432 | NSMUSG00000024037 | 7773 | .437502 | .14745 | .97 |
| Exosc10 | hr4: 147932535-147956509 | NSMUSG00000017264 | 0912 | .24087 | .418208 | .97 |
| Strc | hr2: 121184376-121206676 | NSMUSG00000033498 | 40476 | .193516 | .0655076 | .95 |
| 1110004E09Rik | hr16: 90926055-90935094 | NSMUSG00000022972 | 8001 | .160412 | .0543454 | .95 |
| Tusc3 | hr8: 40068920-40250468 | NSMUSG00000039530 | 0286 | .165876 | .0562185 | .95 |
| 1700001O22Rik | hr2: 30651082-30659184 | NSMUSG00000044320 | 3598 | .280819 | .0952251 | .95 |
| Olfr919 | hr9: 38505013-38505961 | NSMUSG00000056961 | 58432 | .685814 | .23276 | .95 |
| Cplx2 | hr13: 54472712-54485278 | NSMUSG00000025867 | 2890 | .295693 | .1004 | .95 |
| Ptchd1 | hrX: 152004278-152057870 | NSMUSG00000041552 | 11612 | .0894013 | .0303599 | .94 |
| H2-Ob | hr17: 34375849-34382853 | NSMUSG00000041538 | 5002 | .103351 | .0351652 | .94 |
| BC048671 | hr6: 90251263-90255442 | NSMUSG00000049694 | 43535 | .367998 | .125432 | .93 |
| Col4a3 | hr1: 82583495-82718634 | NSMUSG00000079465 | 2828 | .0378872 | .0129295 | .93 |
| C3 | hr17: 57343395-57367559 | NSMUSG00000024164 | 00048759 | .0472419 | .0161239 | .93 |
| Nid2 | hr14: 20570478-20643045 | NSMUSG00000021806 | 8074 | .0582554 | .0198916 | .93 |
| Mast4 | hr13: 103522568-104124572 | NSMUSG00000034751 | 28329 | .621121 | .212199 | .93 |
| BC024659 | hr13: 41345212-41371946 | NSMUSG00000091264 | 08934 | .87286 | .29898 | .92 |
| Eno2 | hr6: 124710072-124719527 | NSMUSG00000004267 | 3807 | .74732 | .255993 | .92 |
| 4930556J02Rik | hr14: 62111524-62119219 | A | A | .446276 | .15317 | .91 |
| Mblac1 | hr5: 138635541-138636849 | NSMUSG00000049285 | 30216 | 1.8185 | 4.3606 | .91 |
| Cdkn1c | hr7: 150644243-150646955 | NSMUSG00000037664 | 2577 | .160303 | .0550708 | .91 |
| Ythdc1 | hr5: 87233514-87265682 | NSMUSG00000035851 | 31386 | .19962 | .755883 | .91 |
| Acpl2 | hr9: 96723761-96789841 | NSMUSG00000043587 | 35534 | .154324 | .0530476 | .91 |
| Gas6 | hr8: 13465373-13494535 | NSMUSG00000031451 | 4456 | .114429 | .0393345 | .91 |
| Fbln7 | hr2: 128689667-128722770 | NSMUSG00000027386 | 0370 | .176786 | .0608372 | .91 |
| Evi2a, Evi2b | hr11: 79153393-79395111 | A | A | .87001 | .33387 | .90 |
| Taf9b | hrX: 103402212-103416497 | NSMUSG00000047242 | 07786 | .497312 | .171456 | .90 |
| Batf | hr12: 87027669-87050037 | NSMUSG00000034266 | 3314 | .242079 | .0837069 | .89 |
| Spock1 | hr13: 57522555-58009693 | NSMUSG00000056222 | 0745 | .0692883 | .0239656 | .89 |
| Ifit3 | hr19: 34658018-34663472 | NSMUSG00000074896 | 5959 | .694053 | .240095 | .89 |
| Phactr1 | hr13: 42775991-43233881 | NSMUSG00000054728 | 18194 | .069782 | .0242151 | .88 |
| 6-Sep | hr9: 25060039-25116156 | NSMUSG00000001833 | 35072 | .57138 | .545382 | .88 |
| Abca12 | hr1: 71289663-71461484 | NSMUSG00000050296 | 4591 | .0815514 | .0283114 | .88 |
| NA | hr3: 144087033-144088215 | A | A | .294061 | .10209 | .88 |
| NA | hr5: 28527370-28527962 | A | A | .738432 | .256364 | .88 |
| NA | hr7: 134804854-134810084 | A | A | .0573353 | .0199053 | .88 |
| NA | hr6: 30123409-30124191 | A | A | .496711 | .172445 | .88 |
| NA | hr3: 79009881-79011264 | A | A | .244032 | .0847215 | .88 |
| NA | hr4: 94137755-94139138 | A | A | .244032 | .0847215 | .88 |
| NA | hr5: 149759277-149760544 | A | A | .270601 | .0939456 | .88 |
| NA | hr11: 68210101-68211744 | A | A | .200014 | .0694397 | .88 |
| NA | hr11: 120086659-120092951 | A | A | .0473376 | .0164344 | .88 |
| NA | hr6: 100518068-100520507 | A | A | .128856 | .0447355 | .88 |
| NA | hr11: 69184165-69185263 | A | A | .321616 | .111657 | .88 |
| NA | hr10: 94861606-94863556 | A | A | .164894 | .0572471 | .88 |
| NA | hr6: 31097604-31097886 | A | A | 0.0003 | .47186 | .88 |
| Slc25a2 | hr18: 37089938-38001526 | NSMUSG00000050304 | 3885 | .187032 | .0649955 | .88 |
| Stra8 | hr6: 34870959-34889342 | NSMUSG00000029848 | 0899 | .173832 | .0604321 | .88 |
| Nrk | hrX: 135448968-135543629 | NSMUSG00000052854 | 7206 | .0351427 | .0122191 | .88 |
| 2310035C23Rik | hr1: 107560437-107651708 | NSMUSG00000026319 | 27446 | .0564239 | .0196256 | .88 |
| Cacna1b | hr2: 24461894-24618672 | NSMUSG00000004113 | 2287 | .466231 | .16242 | .87 |
| Mir744 | hr11: 65501745-65601799 | NSMUSG00000076460 | A | 42.621 | 23.937 | .87 |
| Rrp9 | hr9: 106379639-106387746 | NSMUSG00000041506 | 7966 | .09529 | .381811 | .87 |
| Zfp192 | hr13: 21605089-21622983 | NSMUSG00000063894 | 3681 | 5.2024 | 5.7616 | .87 |
| Cab39l | hr14: 60059817-60167740 | NSMUSG00000021981 | 9008 | .386597 | .134899 | .87 |
| Tktl2 | hr8: 69035638-69043098 | NSMUSG00000025519 | 4419 | .197518 | .0689792 | .86 |
| Ryr3 | hr2: 112471538-112870488 | NSMUSG00000057378 | 0192 | .086407 | .030248 | .86 |
| FR117877 | hr3: 68808893-68838545 | A | A | 4929.5 | 2242.3 | .85 |
| Mvk | hr5: 114894314-114910599 | NSMUSG00000041939 | 7855 | .389552 | .136778 | .85 |
| Fgfr2 | hr7: 137305964-137410322 | NSMUSG00000030849 | 4183 | .0567137 | .0199187 | .85 |
| Plekha1 | hr7: 138009423-138056816 | NSMUSG00000040268 | 01476 | .476204 | .167405 | .84 |
| Srrm4 | hr5: 116888730-117041826 | NSMUSG00000063919 | 8955 | .0295429 | .0104232 | .83 |
| Fam188b | hr6: 55153376-55270216 | NSMUSG00000038022 | 30323 | .046188 | .0163063 | .83 |
| H2-T10 | hr17: 36252815-36258389 | NSMUSG00000079491 | 5024 | .0867101 | .0306155 | .83 |
| Dpy19l2 | hr9: 24361491-24500737 | NSMUSG00000085576 | 20752 | .265298 | .0936824 | .83 |
| Mirlet7c-1 | hr16: 77599901-77599995 | NSMUSG00000065557 | A | 3973.2 | 2036.1 | .82 |
| 4933408J17Rik | hr10: 93003376-93067990 | A | A | .330637 | .117317 | .82 |
| Ajap1 | hr4: 152747329-152856939 | NSMUSG00000039546 | 30959 | .307151 | .109002 | .82 |
| Col5a2 | hr1: 45431175-45560127 | NSMUSG00000026042 | 2832 | .0387041 | .0137533 | .81 |
| Cryz | hr3: 154239483-154286146 | NSMUSG00000028199 | 2972 | .120585 | .0429214 | .81 |
| Dr1 | hr5: 108697915-108709540 | NSMUSG00000029265 | 3486 | .67946 | .599322 | .80 |
| Gemin6 | hr17: 80623828-80627837 | NSMUSG00000055760 | 21705 | .74888 | .62424 | .80 |
| Mpg | hr11: 32126504-32167614 | NSMUSG00000020287 | 68395 | .582668 | .207996 | .80 |
| Slc7a11 | hr3: 50168857-50247535 | NSMUSG00000027737 | 6570 | .0323 | .0844 | .80 |
| Otog | hr7: 53496356-53566804 | NSMUSG00000009487 | 8419 | .262733 | .0939846 | .80 |
| 2610015P09Rik | hr16: 43890014-43964427 | NSMUSG00000022701 | 12153 | .636697 | .227792 | .80 |
| H2-Aa | hr17: 34419695-34424716 | NSMUSG00000036594 | 4960 | .373938 | .133787 | .80 |
| Krt15 | hr11: 99993072-99997263 | NSMUSG00000054146 | 6665 | .112461 | .0402446 | .79 |
| Anxa11 | hr14: 26661640-26706290 | NSMUSG00000021866 | 00039503 | .545079 | .195088 | .79 |
| Rrp8 | hr7: 112880243-112892852 | NSMUSG00000030888 | 01867 | .702165 | .251491 | .79 |
| Cp | hr3: 19857053-19935310 | NSMUSG00000003617 | 2870 | .141304 | .0506376 | .79 |
| Ebf3 | hr7: 144385353-144506128 | NSMUSG00000010476 | 3593 | .847017 | .303757 | .79 |
| Ophn1 | hrX: 95752853-96086324 | NSMUSG00000031214 | 4190 | .0634238 | .0227459 | .79 |
| Mmp10 | hr9: 7502359-7510240 | NSMUSG00000047562 | 7384 | .14226 | .0510251 | .79 |
| Cyp4f17 | hr17: 32643406-32665839 | NSMUSG00000091586 | 08285 | .186773 | .0670097 | .79 |
| Ccdc136 | hr6: 29348925-29376995 | NSMUSG00000029769 | 32664 | .101471 | .0364335 | .79 |
| Tub | hr7: 116154393-116226838 | NSMUSG00000031028 | 2141 | .0310169 | .0111383 | .78 |
| Myo1d | hr11: 80295628-80593527 | NSMUSG00000035441 | 38367 | .0947654 | .0340478 | .78 |
| Ankrd27 | hr7: 36371265-36424256 | NSMUSG00000034867 | 45886 | .27335 | .457626 | .78 |
| Fgd6 | hr10: 93498745-93608084 | NSMUSG00000020021 | 3998 | .41822 | .150316 | .78 |
| Stmn1-rs1 | hr9: 115151699-115219539 | A | A | .835685 | .300515 | .78 |
| Cgnl1 | hr9: 71474315-71619409 | NSMUSG00000032232 | 8178 | .0315251 | .0113474 | .78 |
| Afap1l2 | hr19: 56986843-57083065 | NSMUSG00000025083 | 26250 | .825 | .09736 | .78 |
| Nedd8 | hr14: 56281103-56290743 | NSMUSG00000010376 | 8002 | .24861 | .449902 | .78 |
| Dock11 | hrX: 33428826-33616557 | NSMUSG00000031093 | 5974 | .0998716 | .0360442 | .77 |
| Sv2a | hr3: 95985149-95999103 | NSMUSG00000038486 | 4051 | .224256 | .0810046 | .77 |
| Agpat9 | hr5: 101275247-101328121 | NSMUSG00000029314 | 31510 | .201824 | .0729025 | .77 |
| Mirlet7f-2 | hrX: 148237824-148369961 | NSMUSG00000065602 | A | 99849 | 2192.9 | .77 |
| Gadd45g | hr13: 51942043-51943843 | NSMUSG00000021453 | 3882 | .78884 | .37288 | .76 |
| Snora19_dup2 | hr19: 60837018-60866596 | A | A | 4143.4 | 134.16 | .75 |
| Npsr1 | hr9: 23902461-24120842 | NSMUSG00000043659 | 19239 | .0571921 | .0207688 | .75 |
| Csmd3 | hr15: 47412183-48623535 | NSMUSG00000022311 | 39420 | .0191414 | .00695561 | .75 |
| Grhl3 | hr4: 135097802-135129535 | NSMUSG00000037188 | 30824 | .094192 | .0342279 | .75 |
| Prdm9 | hr17: 15680042-15700287 | NSMUSG00000051977 | 13389 | .587756 | .213976 | .75 |
| Hk1 | hr10: 61731602-61842656 | NSMUSG00000037012 | 5275 | .577736 | .210333 | .75 |
| Foxc2 | hr8: 123640070-123642794 | NSMUSG00000046714 | 4234 | .132471 | .0482494 | .75 |
| Htr7 | hr19: 36033218-36131850 | NSMUSG00000024798 | 5566 | .0925449 | .0337416 | .74 |
| Bcr | hr10: 74523640-74647668 | NSMUSG00000009681 | 10279 | .461923 | .168917 | .73 |
| Pde3b | hr7: 121558767-121681451 | NSMUSG00000030671 | 8576 | .0954693 | .0349523 | .73 |
| Ptk6 | hr2: 180929828-180937494 | NSMUSG00000038751 | 0459 | .123439 | .0452061 | .73 |
| Pard3b | hr1: 61685397-62688858 | NSMUSG00000052062 | 2823 | .0267082 | .00978357 | .73 |
| Afap1l1 | hr18: 61889915-61946316 | NSMUSG00000033032 | 06877 | .13351 | .0489373 | .73 |
| Mtus1 | hr8: 42076265-42219080 | NSMUSG00000045636 | 02103 | .0380424 | .0139483 | .73 |
| Cmya5 | hr13: 93810669-93914679 | NSMUSG00000047419 | 6469 | .0514972 | .0188942 | .73 |
| Lig3 | hr11: 82594636-82684712 | NSMUSG00000020697 | 6882 | .382698 | .140432 | .73 |
| Col8a1 | hr16: 57624368-57754850 | NSMUSG00000068196 | 2837 | .0530645 | .0194746 | .72 |
| Prss50 | hr9: 110760470-110767132 | NSMUSG00000048752 | 35631 | .187322 | .0688586 | .72 |
| Ikbke | hr1: 133151178-133176140 | NSMUSG00000042349 | 6489 | .591469 | .217646 | .72 |
| Fam149a | hr8: 46422068-46467645 | NSMUSG00000070044 | 12326 | .0492578 | .0181508 | .71 |
| Arl2 | hr19: 6134388-6141137 | NSMUSG00000024944 | 6327 | .22743 | .820876 | .71 |
| Wdr52 | hr16: 44394911-44482541 | NSMUSG00000071550 | 12517 | .0680286 | .0250782 | .71 |
| Epha8 | hr4: 136485333-136512731 | NSMUSG00000028661 | 3842 | .0507558 | .0187146 | .71 |
| BC090627, Chkb, Cpt1b | hr15: 89246835-89260358 | A | A | .103096 | .0380586 | .71 |
| Rbm17 | hr2: 11507065-11524826 | NSMUSG00000037197 | 6938 | .355359 | .131245 | .71 |
| Egr1 | hr18: 35020860-35024610 | NSMUSG00000038418 | 3653 | .77473 | .657234 | .70 |
| Hydin | hr8: 112790876-113134153 | NSMUSG00000059854 | 44653 | .0156099 | .00578122 | .70 |
| 5430421F17Rik | hr8: 26413121-26416742 | A | A | .134652 | .0498985 | .70 |
| Mpc2 | hr1: 167391338-167411345 | A | A | .49328 | .554108 | .69 |
| Ghitm | hr14: 37933631-37948508 | NSMUSG00000041028 | 6092 | .680018 | .252868 | .69 |
| Yes1 | hr5: 32913543-32989439 | NSMUSG00000014932 | 2612 | .0674139 | .0250885 | .69 |
| Pcdh19 | hrX: 130117399-130223532 | NSMUSG00000051323 | 79653 | .873662 | .325269 | .69 |
| Smpd3 | hr8: 108776447-108861888 | NSMUSG00000031906 | 8994 | .0459126 | .0171374 | .68 |
| Sh2d2a | hr3: 87650676-87659644 | NSMUSG00000028071 | 7371 | .0560678 | .0209467 | .68 |
| Ncam1 | hr9: 49310250-49607174 | NSMUSG00000039542 | 7967 | .0429619 | .0160568 | .68 |
| Mir301 | hr11: 86922762-86936476 | NSMUSG00000065589 | A | 1122.4 | 2853.7 | .67 |
| Bend5 | hr4: 110070395-111330115 | NSMUSG00000028545 | 7621 | .158223 | .0592041 | .67 |
| Sema5b | hr16: 35541447-35664344 | NSMUSG00000052133 | 0357 | .302514 | .113434 | .67 |
| Nr1h2 | hr7: 51805002-51809293 | NSMUSG00000060601 | 2260 | .64624 | .617456 | .67 |
| Snora65 | hr2: 32817231-32819565 | NSMUSG00000065124 | A | 12.566 | 17.413 | .66 |
| 4831440E17Rik | hr5: 25005614-25010291 | A | A | .063688 | .0239928 | .65 |
| Map3k5 | hr10: 19654331-19862556 | NSMUSG00000071369 | 6408 | .50134 | .944633 | .65 |
| Sez6l | hr5: 112848170-113006218 | NSMUSG00000058153 | 6747 | .0800417 | .0302873 | .64 |
| Daam1 | hr12: 72932064-73093354 | NSMUSG00000034574 | 08846 | .408188 | .154838 | .64 |
| Mirlet7a-1 | hr13: 48633547-48633641 | NSMUSG00000065421 | A | 775.03 | 433.11 | .63 |
| Rxfp3 | hr15: 10963471-10967723 | NSMUSG00000060735 | 39336 | .0664896 | .025244 | .63 |
| Slc6a13 | hr6: 121250313-121287736 | NSMUSG00000030108 | 4412 | .902634 | .343101 | .63 |
| Mrpl11 | hr19: 4962305-4966995 | NSMUSG00000024902 | 6419 | .287967 | .109746 | .62 |
| Mir30a | hr1: 23279107-23279178 | NSMUSG00000065405 | A | 7696 | 4371.4 | .62 |
| Ulk1 | hr5: 111213507-111239100 | NSMUSG00000029512 | 2241 | .349792 | .133366 | .62 |
| Oprd1 | hr4: 131666640-131700401 | NSMUSG00000050511 | 8386 | .0727912 | .0277666 | .62 |
| Pax1 | hr2: 147190729-147200784 | NSMUSG00000037034 | 8503 | .139408 | .0532571 | .62 |
| Stk36 | hr1: 74648028-74683467 | NSMUSG00000033276 | 69209 | .0462999 | .0176943 | .62 |
| Dnajc18 | hr18: 35830758-35862798 | NSMUSG00000024350 | 6594 | .222288 | .0849662 | .62 |
| Pik3c2g | hr6: 139535771-139917804 | NSMUSG00000030228 | 8705 | .176343 | .0674225 | .62 |
| Gtpbp10 | hr5: 5537456-5559501 | NSMUSG00000040464 | 07704 | .469683 | .179722 | .61 |
| Cpeb1 | hr7: 88491911-88599644 | NSMUSG00000025586 | 2877 | .104203 | .0398995 | .61 |
| Tfcp2l1 | hr1: 120524521-120581745 | NSMUSG00000026380 | 1879 | .156969 | .0601513 | .61 |
| Gtpbp2 | hr17: 46297980-46306319 | NSMUSG00000023952 | 6055 | .53098 | .586739 | .61 |
| Ms4a1 | hr19: 11271306-11374374 | NSMUSG00000024673 | 2482 | .16048 | .0615117 | .61 |
| AI662270 | hr11: 83037077-83040086 | NSMUSG00000087107 | A | 2.0254 | .61486 | .61 |
| Pias1 | hr9: 62727883-62828686 | NSMUSG00000032405 | 6469 | .35501 | .136282 | .60 |
| Mir19b-2 | hrX: 50095159-50095243 | NSMUSG00000065473 | A | 7233.2 | 4331.6 | .60 |
| Tubgcp4 | hr2: 120996941-121097122 | NSMUSG00000027263 | 1885 | .421772 | .162429 | .60 |
| Supt6h | hr11: 78020250-78059205 | NSMUSG00000002052 | 0926 | .367674 | .141666 | .60 |
| Myh10 | hr11: 68505416-68630126 | NSMUSG00000020900 | 7579 | .0241416 | .00933684 | .59 |
| Clrn3 | hr7: 142703138-142720337 | NSMUSG00000050866 | 12070 | .73615 | .674587 | .57 |
| Megf6 | hr4: 153544821-153649830 | NSMUSG00000057751 | 30971 | .252253 | .0980423 | .57 |
| Rap1gap | hr4: 137220640-137285776 | NSMUSG00000041351 | 10351 | .180682 | .070297 | .57 |
| Fn1 | hr1: 71632096-71699745 | NSMUSG00000026193 | 4268 | .0948365 | .0369069 | .57 |
| Klhl24 | hr16: 20097626-20127817 | NSMUSG00000062901 | 5785 | .731038 | .284557 | .57 |
| Mdm4 | hr1: 134886421-134921925 | NSMUSG00000054387 | 7248 | .135386 | .0527053 | .57 |
| Zcchc16 | hrX: 141123449-141556953 | NSMUSG00000071679 | 19287 | .0679821 | .0264802 | .57 |
| Miat | hr5: 112642247-112657968 | A | A | .025038 | .00975758 | .57 |
| Oxtr | hr6: 112423677-112439802 | NSMUSG00000049112 | 8430 | .0562185 | .0219305 | .56 |
| Dio2 | hr12: 91962991-91976878 | NSMUSG00000007682 | A | .0405021 | .0158089 | .56 |
| Ppp1r2 | hr16: 31251626-31275363 | NSMUSG00000047714 | 6849 | .400471 | .156358 | .56 |
| En2 | hr5: 28492235-28498706 | NSMUSG00000039095 | 3799 | .0917959 | .0358462 | .56 |
| Malt1 | hr18: 65590650-65638446 | NSMUSG00000032688 | 40354 | .785944 | .307003 | .56 |
| Adprm | hr11: 66851381-66866120 | A | A | .609108 | .238144 | .56 |
| Sarm1 | hr11: 78285831-78311256 | NSMUSG00000050132 | 37868 | .608531 | .237945 | .56 |
| Ttc17 | hr2: 94140922-94246846 | NSMUSG00000027194 | 4569 | .507388 | .198551 | .56 |
| Lemd1 | hr1: 134088012-134153959 | NSMUSG00000079330 | 13409 | .0255 | .401586 | .55 |
| Usp5 | hr6: 124765036-124779465 | NSMUSG00000038429 | 2225 | .45116 | .568285 | .55 |
| Samd7 | hr3: 30645214-30666096 | NSMUSG00000051860 | 5953 | .49676 | .37167 | .55 |
| Lrr1 | hr12: 70269800-70279997 | NSMUSG00000034883 | 9706 | .237518 | .0933652 | .54 |
| Insm1 | hr2: 146047732-146050754 | NSMUSG00000068154 | 3626 | .632255 | .248601 | .54 |
| Sox5 | hr6: 143776944-144158088 | NSMUSG00000041540 | 0678 | .0291888 | .0114838 | .54 |
| Gab1 | hr8: 83288332-83404378 | NSMUSG00000031714 | 4388 | .0568615 | .0223873 | .54 |
| E530011L22Rik | hr9: 121628298-121669061 | A | A | .0855394 | .0337139 | .54 |
| Vegfc | hr8: 55162885-55271808 | NSMUSG00000031520 | 2341 | .14089 | .0556051 | .53 |
| Ccdc39 | hr3: 33699116-33743232 | NSMUSG00000027676 | 1938 | .0543875 | .0214799 | .53 |
| Rnf152 | hr1: 107173493-107253287 | NSMUSG00000047496 | 20311 | .0287489 | .0113569 | .53 |
| Ndrg3 | hr2: 156753077-156817847 | NSMUSG00000027634 | 9812 | .992918 | .392334 | .53 |
| Casp6 | hr3: 129604342-129617020 | NSMUSG00000027997 | 2368 | .14721 | .453538 | .53 |
| Gspt2 | hrX: 91881407-91883900 | NSMUSG00000071723 | 4853 | .110448 | .0436738 | .53 |
| Anxa7 | hr14: 21274482-21299355 | NSMUSG00000021814 | 1750 | .64525 | .650899 | .53 |
| Nup85 | hr11: 115425757-115445238 | NSMUSG00000020739 | 45007 | .837286 | .331273 | .53 |
| C430049B03Rik, Mir322, | hrX: 50406288-50410378 | A | A | 990.54 | 579 | .53 |
| Mir351, Mir503 | | | | | | |
| Mypn | hr10: 62578542-62666700 | NSMUSG00000020067 | 8802 | .0457734 | .0181264 | .53 |
| Rfc4 | hr16: 23107551-23127803 | NSMUSG00000022881 | 06344 | .11794 | .442869 | .52 |
| Homer2 | hr7: 88745366-88851811 | NSMUSG00000025813 | 6557 | .0230476 | .00913341 | .52 |
| Bsph1 | hr7: 14036189-14058798 | NSMUSG00000074378 | 30470 | .308 | .311 | .52 |
| Herc6 | hr6: 57530985-57615130 | NSMUSG00000029798 | 7138 | .0518832 | .0205671 | .52 |
| Atp6v0b | hr4: 117556934-117559934 | NSMUSG00000033379 | 14143 | .87787 | .745587 | .52 |
| Ccdc146 | hr5: 20798778-20930495 | NSMUSG00000064280 | 5172 | .046091 | .0183045 | .52 |
| Ccdc103, Fam187a | hr11: 102742557-102748045 | A | A | .386083 | .153403 | .52 |
| Plxdc1 | hr11: 97784550-97847760 | NSMUSG00000017417 | 2324 | .242611 | .096592 | .51 |
| Rwdd4a | hr8: 48618998-48638191 | NSMUSG00000031568 | 92174 | .123608 | .0492398 | .51 |
| Myo15 | hr11: 60282840-60341870 | NSMUSG00000042678 | 7910 | .237042 | .0944855 | .51 |
| Wnt7a | hr6: 91313976-91361363 | NSMUSG00000030093 | 2421 | .086871 | .0346406 | .51 |
| Zfp28 | hr7: 6336027-6349347 | NSMUSG00000062861 | 2690 | .566193 | .226016 | .51 |
| Gtf2ird2 | hr5: 134659907-134694013 | NSMUSG00000015942 | 14674 | .09937 | .03583 | .50 |
| Katnb1 | hr8: 97605100-97666440 | NSMUSG00000031787 | 4187 | .333028 | .133003 | .50 |
| Ptrf | hr11: 100818050-100831931 | NSMUSG00000004044 | 9285 | .061718 | .0246615 | .50 |
| Uchl5 | hr1: 145624407-145654596 | NSMUSG00000018189 | 6207 | .03603 | .414182 | .50 |
| Mapk7 | hr11: 61302313-61307705 | NSMUSG00000001034 | 3939 | .87376 | .349653 | .50 |
| Srcrb4d | hr5: 136436092-136450346 | NSMUSG00000029699 | 09267 | .502075 | .201253 | .49 |
| Psip1 | hr4: 83101584-83132294 | NSMUSG00000028484 | 01739 | .944904 | .378759 | .49 |
| Col22a1 | hr15: 71628905-71864657 | NSMUSG00000079022 | 9700 | .0392258 | .0157317 | .49 |
| Artn | hr4: 117598766-117602368 | NSMUSG00000028539 | 1876 | .268836 | .107824 | .49 |
| Tmem87a | hr2: 120181044-120330655 | NSMUSG00000033808 | 11499 | .08448 | .0339277 | .49 |
| Use1 | hr8: 73890746-73893631 | NSMUSG00000002395 | 7023 | .21818 | .890941 | .49 |
| Ift20 | hr11: 78349937-78354975 | NSMUSG00000001105 | 5978 | .37922 | .553971 | .49 |
| Mir125b-2 | hr16: 77646517-77646588 | NSMUSG00000065472 | A | 4815.2 | 973.1 | .49 |
| Tmem22 | hr9: 100452606-100471504 | NSMUSG00000070287 | 45020 | .132383 | .0532208 | .49 |
| Pmp2 | hr3: 10179850-10183885 | NSMUSG00000052468 | 8857 | .179479 | .0721791 | .49 |
| Ap4e1 | hr2: 126834446-126895550 | NSMUSG00000001998 | 08011 | .213762 | .085967 | .49 |
| AW549542 | hr5: 120020137-120030367 | A | A | .65213 | .28581 | .47 |
| Atad5 | hr11: 79902901-79949293 | NSMUSG00000017550 | 37877 | .411334 | .166366 | .47 |
| Gemin8 | hrX: 162608411-162628444 | NSMUSG00000040621 | 37221 | .54522 | .03116 | .47 |
| Vmn2r45 | hr7: 9638310-9655801 | NSMUSG00000090662 | 00042810 | .0973935 | .0394988 | .47 |
| Al118078 | hr9: 55174755-55286151 | NSMUSG00000032313 | 44886 | .164376 | .0666748 | .47 |
| Duoxa2 | hr2: 122124635-122128621 | NSMUSG00000027225 | 6811 | .427816 | .173604 | .46 |
| Aqp6 | hr15: 99431830-99435908 | NSMUSG00000043144 | 1831 | .355984 | .144489 | .46 |
| BC021785, G630090 | hr10: 39666717-39706452 | A | A | .109104 | .0442846 | .46 |
| E17Rik | | | | | | |
| Lrrn3 | hr12: 41750676-43056573 | NSMUSG00000036295 | 6981 | .13136 | .0533268 | .46 |
| Ccdc177 | hr12: 81856433-81861702 | A | A | .267068 | .108468 | .46 |
| Akap9 | hr5: 3928185-4080204 | NSMUSG00000040407 | 00986 | .0886935 | .0360413 | .46 |
| Gria3 | hrX: 38754480-39031778 | NSMUSG00000001986 | 3623 | .0443366 | .018025 | .46 |
| Pigs | hr11: 78141923-78156278 | NSMUSG00000041958 | 76846 | .225416 | .0916678 | .46 |
| Lin37 | hr7: 31340459-31344665 | NSMUSG00000036845 | 5660 | .18747 | .483208 | .46 |
| 6030458C11Rik | hr15: 12737931-12754412 | NSMUSG00000022195 | 7877 | .204366 | .083283 | .45 |
| Fam167a | hr14: 64055230-64084339 | NSMUSG00000035095 | 19148 | .0559383 | .0227974 | .45 |
| Iba57 | hr11: 58968870-58977247 | A | A | .15599 | .471338 | .45 |
| Skp1a | hr11: 52045496-52060360 | NSMUSG00000036309 | 1402 | .968048 | .395049 | .45 |
| Ern1 | hr11: 106258933-106349110 | NSMUSG00000020715 | 8943 | .198703 | .0812301 | .45 |
| Spty2d1 | hr7: 54245765-54263784 | NSMUSG00000049516 | 01685 | .36816 | .559727 | .44 |
| Dnalc1 | hr12: 85455277-85484460 | NSMUSG00000042523 | 05000 | .377338 | .154374 | .44 |
| Ltv1 | hr10: 12898443-12912943 | NSMUSG00000019814 | 53258 | .0799056 | .0326943 | .44 |
| Tubb3 | hr8: 125935463-125945910 | NSMUSG00000062380 | 2152 | .104083 | .0425913 | .44 |
| Heatr7b1 | hr1: 90123594-90174154 | NSMUSG00000079429 | 00040766 | .0548985 | .0224734 | .44 |
| Gm15663 | hr10: 105011606-105020926 | NSMUSG00000085282 | A | .0711295 | .0291372 | .44 |
| Ghsr | hr3: 27270272-27276932 | NSMUSG00000051136 | 08188 | .36226 | .148491 | .44 |
| Slx4ip | hr2: 136699516-136895514 | A | A | .180434 | .0739652 | .44 |
| Mpp2 | hr11: 101918330-101949829 | NSMUSG00000017314 | 0997 | .37518 | .564317 | .44 |
| Krtcap2 | hr3: 89050359-89053644 | NSMUSG00000042747 | 6059 | .17393 | .481783 | .44 |
| Carm1 | hr9: 21351337-21400414 | NSMUSG00000032185 | 9035 | .523092 | .214688 | .44 |
| Sstr4 | hr2: 148221112-148222500 | NSMUSG00000037014 | 0608 | .899508 | .369338 | .44 |
| Fam217a | hr13: 35001832-35011861 | A | A | .0867866 | .0357042 | .43 |
| Kcng1 | hr2: 168087197-168094831 | NSMUSG00000074575 | 41794 | .206425 | .0849785 | .43 |
| Carkd | hr8: 11497505-11513286 | NSMUSG00000031505 | 9225 | .64917 | .6793 | .43 |
| Sprr1b | hr3: 92240730-92242701 | NSMUSG00000048455 | 0754 | .458139 | .188953 | .42 |
| Sec63 | hr10: 42481301-42552320 | NSMUSG00000019802 | 40740 | .43502 | .591928 | .42 |
| C230079O03Rik | hr7: 143523898-143541098 | A | A | 7.9018 | .38612 | .42 |
| Pot1b | hr17: 55791322-55851926 | NSMUSG00000024174 | 2836 | .0658332 | .0271813 | .42 |
| Lipk | hr19: 34082743-34122393 | NSMUSG00000024771 | 40633 | .0953698 | .0394438 | .42 |
| Myom3 | hr4: 135315629-135371479 | NSMUSG00000037139 | 42702 | .035728 | .0147996 | .41 |
| AU022252 | hr4: 118897742-118905329 | NSMUSG00000078584 | 30696 | .266738 | .110512 | .41 |
| Spata31d1b | hr13: 59813644-59820650 | A | A | .159175 | .0659854 | .41 |
| Arhgef9 | hrX: 92244282-92361825 | NSMUSG00000025656 | 36915 | .0463237 | .0192127 | .41 |
| Stox2 | hr8: 48265401-48437702 | NSMUSG00000038143 | 1069 | .108224 | .0449407 | .41 |
| Orc5 | hr5: 21992306-22056149 | NSMUSG00000029012 | 6429 | .669723 | .278163 | .41 |
| Syt15 | hr14: 35033231-35043607 | NSMUSG00000041479 | 19508 | .069392 | .0288782 | .40 |
| Chmp3 | hr6: 71493847-71531568 | NSMUSG00000053119 | 6700 | .873562 | .364002 | .40 |
| BC055402 | hr1: 57257488-57271840 | A | A | .0691969 | .0288362 | .40 |
| Havcr1 | hr11: 46553724-46593080 | NSMUSG00000040405 | 71283 | .262768 | .10979 | .39 |
| Card10 | hr15: 78605565-78633472 | NSMUSG00000033170 | 05844 | .189918 | .0793704 | .39 |
| Wnt2b | hr3: 104747722-104764627 | NSMUSG00000027840 | 2414 | .0651216 | .0272423 | .39 |
| Ddx31 | hr2: 28695925-28761095 | NSMUSG00000026806 | 27674 | .546002 | .228415 | .39 |
| Helt | hr8: 47377401-47380025 | NSMUSG00000047171 | 34219 | .699788 | .292774 | .39 |
| Odc1 | hr12: 17551678-17558308 | NSMUSG00000011179 | 8263 | 5.3052 | 4.8112 | .38 |
| Mapk10 | hr5: 103336966-103640353 | NSMUSG00000046709 | 6414 | .0722536 | .0303314 | .38 |
| Eif5 | hr12: 112776311-112784964 | NSMUSG00000021282 | 00047658 | .474301 | .199415 | .38 |
| Palm2 | hr4: 57581119-57730000 | NSMUSG00000090053 | 42481 | .031725 | .0133409 | .38 |
| Pnma5 | hrX: 70279319-70282442 | NSMUSG00000050424 | 85377 | .0903969 | .0380249 | .38 |
| 2900057B20Rik | hr18: 75979831-76308218 | A | A | .195048 | .0820484 | .38 |
| Mir704, Pdia4 | hr6: 47746139-47763511 | A | A | .00876 | .424544 | .38 |
| Selk | hr14: 30781565-30788260 | NSMUSG00000042682 | A | .526337 | .221535 | .38 |
| Pak7 | hr2: 135906823-136213703 | NSMUSG00000039913 | 41656 | .221168 | .0931183 | .38 |
| Ptpn2 | hr18: 67825154-67884275 | NSMUSG00000024539 | 9255 | .292909 | .123714 | .37 |
| Ddx50 | hr10: 62078770-62113946 | NSMUSG00000020076 | 4213 | .40527 | .01741 | .36 |
| Dmrt2 | hr19: 25746900-25753481 | NSMUSG00000048138 | 26049 | .114743 | .0485823 | .36 |
| Gm3646 | hr1: 39860985-39862177 | NSMUSG00000091937 | 00042065 | .14425 | .484528 | .36 |
| Vps52 | hr17: 34092826-34103433 | NSMUSG00000024319 | 24705 | .02789 | .435786 | .36 |
| Pigf | hr17: 87362450-87424741 | NSMUSG00000024145 | 8701 | .241555 | .102448 | .36 |
| Cox11 | hr11: 90499497-90548915 | NSMUSG00000020544 | 9802 | .26264 | .535558 | .36 |
| Ppfibp2 | hr7: 114738564-114901546 | NSMUSG00000036528 | 9024 | .0495456 | .0210171 | .36 |
| Lias | hr5: 65782735-65800446 | NSMUSG00000029199 | 9464 | .778791 | .330823 | .35 |
| Ankrd34b | hr13: 93195923-93211613 | NSMUSG00000045034 | 18440 | .0684431 | .0291017 | .35 |
| Lsm14b | hr2: 179759691-179770166 | NSMUSG00000039108 | 41846 | .55652 | .662488 | .35 |
| Tmprss11g | hr5: 86914901-86947625 | NSMUSG00000079451 | 20454 | .0940203 | .040065 | .35 |
| Dach2 | hrX: 110411864-110949995 | NSMUSG00000025592 | 3837 | .0748 | .0319128 | .34 |
| Ptger2 | hr14: 45607785-45623495 | NSMUSG00000037759 | 9217 | .0685503 | .0292585 | .34 |
| Nrg2 | hr18: 36177311-36356814 | NSMUSG00000060275 | 00042150 | .0404832 | .0172912 | .34 |
| NA | hr4: 124316287-124317721 | A | A | .52057 | .649724 | .34 |
| NA | hr8: 18589060-18589944 | A | A | .49211 | .34673 | .34 |
| Cngb1 | hr5: 143200557-143579066 | NSMUSG00000031789 | 33329 | .0866488 | .0370959 | .34 |
| E230008N13Rik | hr4: 45903174-45963646 | NSMUSG00000035539 | 81522 | .0959397 | .0411174 | .33 |
| 2610008E11Rik | hr10: 78527118-78560345 | NSMUSG00000060301 | 2128 | .0959609 | .0411367 | .33 |
| Gm16516 | hr11: 60731323-60745369 | NSMUSG00000042549 | A | .295899 | .126888 | .33 |
| Snord92 | hr17: 71965554-72008371 | NSMUSG00000093289 | A | 8973.8 | 157.54 | .33 |
| Tbx5 | hr5: 120284671-120335227 | NSMUSG00000018263 | 1388 | .0600637 | .0258616 | .32 |
| NA | hr9: 96796019-96796805 | A | A | .15302 | .08277 | .32 |
| Bag2 | hr1: 33802328-33814595 | NSMUSG00000042215 | 13539 | .07141 | .462373 | .32 |
| Serpinb10 | hr1: 109425579-109445848 | NSMUSG00000092572 | 41197 | .0805271 | .0348161 | .31 |
| Spnb2 | hr11: 29999394-30119772 | NSMUSG00000020315 | 0742 | .112968 | .0488719 | .31 |
| Casr | hr16: 36493781-36562220 | NSMUSG00000051980 | 2374 | .081363 | .0352439 | .31 |
| Gm10778_dup2 | hr10: 81274930-81289235 | A | A | .0471877 | .0204434 | .31 |
| Nfkbia | hr12: 56590395-56593634 | NSMUSG00000021025 | 8035 | .47452 | .10656 | .31 |
| Ddx25 | hr9: 35349432-35366055 | NSMUSG00000032101 | 0959 | .819576 | .355439 | .31 |
| Dennd1a | hr2: 37631768-38142904 | NSMUSG00000035392 | 27801 | .377704 | .163821 | .31 |
| Hmg20a | hr9: 56266652-56344743 | NSMUSG00000032329 | 6867 | .0563874 | .0244583 | .31 |
| Scn3b | hr9: 40076800-40099203 | NSMUSG00000049281 | 35281 | .0570325 | .0247495 | .30 |
| NA | hr2: 94018193-94018926 | A | A | .17004 | .941727 | .30 |
| Nr1h3 | hr2: 91024217-91035273 | NSMUSG00000002108 | 2259 | .692211 | .300414 | .30 |
| Amz1 | hr5: 141200080-141229266 | NSMUSG00000050022 | 31842 | .4551 | .631935 | .30 |
| Olfr169 | hr16: 19566032-19566974 | NSMUSG00000068535 | 58158 | .392156 | .17036 | .30 |
| Gzmm | hr10: 79151764-79158005 | NSMUSG00000054206 | 6904 | .620498 | .269735 | .30 |
| Diras2 | hr13: 52599743-52626205 | NSMUSG00000047842 | 8203 | .419885 | .182571 | .30 |
| Nkd1 | hr8: 91045242-91118786 | NSMUSG00000031661 | 3960 | .110037 | .0479715 | .29 |
| Gm15880 | hr7: 87731517-87833763 | NSMUSG00000084821 | A | .0998955 | .0435677 | .29 |
| Dsel | hr1: 113755278-113761495 | NSMUSG00000038702 | 19901 | .328295 | .143206 | .29 |
| Grin2b | hr6: 135679822-136123529 | NSMUSG00000030209 | 4812 | .487587 | .21305 | .29 |
| Hcn1 | hr13: 118391126-118769835 | NSMUSG00000021730 | 5165 | .0496721 | .0217047 | .29 |
| Urod | hr4: 116662821-116666980 | NSMUSG00000028684 | 2275 | .730243 | .31914 | .29 |
| B3galt2 | hr1: 145454628-145549814 | NSMUSG00000033849 | 6878 | .230739 | .100869 | .29 |
| Cnih2 | hr19: 5088537-5098418 | NSMUSG00000024873 | 2794 | .261719 | .114629 | .28 |
| Nmt2 | hr2: 3201559-3243641 | NSMUSG00000026643 | 8108 | .207266 | .0908055 | .28 |
| Srsf11 | hr3: 157673456-157699603 | NSMUSG00000055436 | 9207 | .52013 | .54347 | .28 |
| Arpp21 | hr9: 111967594-112251429 | NSMUSG00000032503 | 4100 | .0789801 | .0346516 | .28 |
| Zbed3 | hr13: 96095191-96107796 | NSMUSG00000041995 | 2114 | .11536 | .00431 | .28 |
| Mfsd8 | hr3: 40622093-40650776 | NSMUSG00000025759 | 2175 | .646236 | .283924 | .28 |
| Spsb4 | hr9: 96843900-96918774 | NSMUSG00000046997 | 11949 | .190574 | .083809 | .27 |
| Nfe2l3 | hr6: 51382668-51408767 | NSMUSG00000029832 | 8025 | .0812354 | .0357476 | .27 |
| Zswim5 | hr4: 116550006-116661710 | NSMUSG00000033948 | 4464 | .18848 | .0829453 | .27 |
| Elk1 | hrX: 20510520-20527734 | NSMUSG00000009406 | 3712 | .248039 | .109159 | .27 |
| Mtfp1 | hr11: 3991483-3995434 | NSMUSG00000004748 | 7900 | 0.6221 | .67592 | .27 |
| Zfp831 | hr2: 174469034-174536331 | NSMUSG00000050600 | 00043757 | .154886 | .0682422 | .27 |
| Ccdd116 | hr16: 17139156-17144516 | NSMUSG00000022768 | 6872 | .800402 | .35294 | .27 |
| Rsph3a | hr17: 8138478-8172421 | NSMUSG00000073471 | 6832 | .465302 | .205391 | .27 |
| Mir200a | hr4: 155429004-155429094 | NSMUSG00000065400 | A | 342.63 | 800.45 | .26 |
| Tas2r137 | hr6: 40441236-40442238 | NSMUSG00000052850 | 74417 | .531994 | .235428 | .26 |
| 2810004N23Rik | hr8: 127363254-127386929 | NSMUSG00000031984 | 6523 | .292156 | .129379 | .26 |
| Pcdh15 | hr10: 73284614-74112477 | NSMUSG00000046980 | A | .6187 | .60411 | .26 |
| Dvl1 | hr4: 155221520-155237462 | NSMUSG00000029071 | 3542 | .17749 | .522006 | .26 |
| Smim3 | hr18: 60633844-60661637 | A | A | .239202 | .106069 | .26 |
| Slc9a2 | hr1: 40738556-40825730 | NSMUSG00000026062 | 26999 | .0607346 | .0269352 | .25 |
| Tnfrsf1b | hr4: 144802270-144836773 | NSMUSG00000028599 | 1938 | .14847 | .953799 | .25 |
| Serf2 | hr2: 121274963-121282500 | NSMUSG00000074884 | 78702 | .901673 | .400309 | .25 |
| Fzd6 | hr15: 38837825-38869736 | NSMUSG00000022297 | 4368 | .0565817 | .0251217 | .25 |
| Lox | hr18: 52676891-52689362 | NSMUSG00000024529 | 6948 | .0710285 | .0315495 | .25 |
| Hecw2 | hr1: 53863717-54251878 | NSMUSG00000042807 | 29152 | .223906 | .0995037 | .25 |
| Tnk1 | hr11: 69659873-69672232 | NSMUSG00000001583 | 3813 | .0837985 | .0373948 | .24 |
| Psme4 | hr11: 30671774-30780361 | NSMUSG00000040850 | 03554 | .0292544 | .0130763 | .24 |
| Fkbp15 | hr4: 61961375-62021582 | NSMUSG00000066151 | 38355 | .81422 | .811808 | .23 |
| Hhatl | hr9: 121693133-121701625 | NSMUSG00000032523 | 4770 | .250837 | .112552 | .23 |
| Nhs | hrX: 158274199-158597722 | NSMUSG00000059493 | 95727 | .100326 | .0450595 | .23 |
| Nudt22 | hr19: 7067508-7070527 | NSMUSG00000037349 | 8323 | .860128 | .386519 | .23 |
| Atrnl1 | hr19: 57685523-58207830 | NSMUSG00000054843 | 26255 | .053417 | .0240157 | .22 |
| Slitrk4 | hrX: 61522618-61530171 | NSMUSG00000046699 | 45446 | .442663 | .199111 | .22 |
| Jagn1 | hr6: 113392510-113398223 | NSMUSG00000051256 | 7767 | .9722 | .33724 | .22 |
| Gramd1a | hr7: 31915145-31936069 | NSMUSG00000001248 | 2857 | .862531 | .388112 | .22 |
| Birc5 | hr11: 117710550-117717057 | NSMUSG00000017716 | 1799 | .68215 | .307542 | .22 |
| Mat2b | hr11: 40492815-40508705 | NSMUSG00000042032 | 08645 | .365524 | .16492 | .22 |
| Nhsl2 | hrX: 99044723-99287394 | NSMUSG00000079481 | 00042480 | .69806 | .766555 | .22 |
| Sucnr1 | hr3: 59885790-59891488 | NSMUSG00000027762 | 4112 | .129654 | .0586584 | .21 |
| Eif2d | hr1: 133049783-133070048 | NSMUSG00000026427 | 6865 | .679332 | .307538 | .21 |
| Fam92a | hr4: 12080868-12099162 | NSMUSG00000028218 | 8099 | .170782 | .0773942 | .21 |
| Bcap31 | hrX: 70931521-70961514 | NSMUSG00000002015 | 7061 | .93717 | .878619 | .20 |
| Fam82b | hr4: 19502212-19534079 | NSMUSG00000028229 | 6302 | .455641 | .20678 | .20 |
| Dnahc9 | hr11: 65644825-65982053 | NSMUSG00000056752 | 37806 | .024824 | .0112673 | .20 |
| Myh11 | hr16: 14194619-14291501 | NSMUSG00000018830 | 7880 | .0191376 | .00869291 | .20 |
| B430010l23Rik | hr8: 42076265-42219080 | NSMUSG00000084960 | A | .105308 | .047845 | .20 |
| Lrriq4 | hr3: 30523188-30571353 | NSMUSG00000027703 | 8307 | .0957127 | .043533 | .20 |
| Itfg3 | hr17: 26349636-26381187 | NSMUSG00000024187 | 06581 | .846206 | .385083 | .20 |
| Myt1l | hr12: 30213248-30608074 | NSMUSG00000061911 | 7933 | .0233518 | .0106276 | .20 |
| Zfp791 | hr8: 87633065-87646994 | NSMUSG00000074194 | 44556 | .436823 | .198827 | .20 |
| Lgals3bp | hr11: 118254065-118263245 | NSMUSG00000033880 | 9039 | .16693 | .531789 | .19 |
| Hist3h2ba | hr11: 58762412-58763032 | NSMUSG00000056895 | 82522 | 1.3812 | .19144 | .19 |
| Gpr107 | hr2: 31007835-31072087 | NSMUSG00000000194 | 77463 | .901177 | .411376 | .19 |
| Hivep1 | hr13: 42147389-42280395 | NSMUSG00000021366 | 10521 | .416405 | .190108 | .19 |
| Crebrf | hr17: 26852594-26913571 | A | A | .0316232 | .0144849 | .18 |
| Ddr2 | hr1: 171899539-172019075 | NSMUSG00000026674 | 8214 | .024451 | .0112109 | .18 |
| Maml3 | hr3: 51491534-51908928 | NSMUSG00000061143 | 33586 | .355324 | .162918 | .18 |
| Trib2 | hr12: 15798532-15823591 | NSMUSG00000020601 | 17410 | .0570675 | .026187 | .18 |
| Nynrin | hr14: 56472951-56493573 | NSMUSG00000075592 | 77154 | .0262266 | .0120407 | .18 |
| Plekhs1 | hr19: 56536126-56561219 | A | A | .0894472 | .0410763 | .18 |
| Ift80 | hr3: 68696420-68808492 | NSMUSG00000027778 | 8259 | .56771 | .260733 | .18 |
| Pitpnm2 | hr5: 124568698-124666427 | NSMUSG00000029406 | 9679 | .143908 | .0661049 | .18 |
| Rprl2 | hr3: 22150291-22150529 | A | A | 06.604 | 25.56 | .17 |
| Trim21 | hr7: 109706435-109713983 | NSMUSG00000030966 | 0821 | .72 | .331923 | .17 |
| Sox17 | hr1: 4481008-4486494 | NSMUSG00000025902 | 0671 | .0840954 | .0387976 | .17 |
| Snx2 | hr18: 53336018-53380514 | NSMUSG00000034484 | 7804 | .945524 | .436271 | .17 |
| Ei24 | hr9: 36586737-36604978 | NSMUSG00000062762 | 3663 | .08798 | .964815 | .16 |
| Dusp2 | hr2: 127161894-127164113 | NSMUSG00000027368 | 3537 | 4.2069 | .56608 | .16 |
| NA | hr13: 98041717-98043918 | A | A | .216292 | .100121 | .16 |
| NA | hr2: 10260657-10262239 | A | A | .25312 | .580066 | .16 |
| NA | hr10: 109642156-109642709 | A | A | .23057 | .569629 | .16 |
| NA | hr7: 16621483-16628889 | A | A | .0600269 | .0277864 | .16 |
| NA | hr15: 59969007-59971482 | A | A | .190223 | .0880542 | .16 |
| Etl4 | hr2: 20211539-20732162 | NSMUSG00000036617 | 08618 | .153937 | .0713178 | .16 |
| Mab21l3 | hr3: 101616998-101640146 | NSMUSG00000044313 | 42125 | .113113 | .0524895 | .15 |
| Lss | hr10: 75994371-76024971 | NSMUSG00000033105 | 6987 | .792288 | .367714 | .15 |
| Lrrc57 | hr2: 120429973-120446554 | NSMUSG00000027286 | 6606 | .368371 | .171095 | .15 |
| D1Pas1 | hr1: 188791294-188794506 | NSMUSG00000039224 | 10957 | .0531891 | .0247278 | .15 |
| Ptgs2 | hr1: 151947253-151955142 | NSMUSG00000032487 | 9225 | .54573 | .64871 | .15 |
| Prkaca | hr8: 86496876-86520344 | NSMUSG00000005469 | 8747 | .45485 | .21154 | .15 |
| Gpr44 | hr19: 11011649-11023756 | NSMUSG00000034117 | 4764 | .178715 | .0831294 | .15 |
| Tifab | hr13: 56275063-56280246 | NSMUSG00000049625 | 12937 | .0607213 | .0282486 | .15 |
| C730027H18Rik | hr10: 70631484-70644788 | A | A | .35993 | .633009 | .15 |
| Spint1 | hr2: 119063095-119075249 | NSMUSG00000027315 | 0732 | .26234 | .122175 | .15 |
| Ubxn6 | hr17: 56207676-56214412 | NSMUSG00000019578 | 6530 | .516379 | .240622 | .15 |
| Esf1 | hr2: 139945616-139996294 | NSMUSG00000045624 | 6580 | .282939 | .131883 | .15 |
| Phkb | hr8: 88364900-88584541 | NSMUSG00000036879 | 02093 | .801965 | .373993 | .14 |
| Dlk1 | hr12: 110691032-110701546 | NSMUSG00000040856 | 3386 | .396902 | .185125 | .14 |
| Clec4n | hr6: 123179860-123197042 | NSMUSG00000023349 | 6620 | .84763 | .863038 | .14 |
| Dennd3 | hr15: 73342989-73402672 | NSMUSG00000036661 | 05841 | .421622 | .197164 | .14 |
| Gm9125_dup2 | hr3: 93851902-93858807 | A | A | .286223 | .134018 | .14 |
| Nfasc | hr1: 134461266-134638374 | NSMUSG00000026442 | 69116 | .0191859 | .00899858 | .13 |
| Itih5 | hr2: 10075169-10178156 | NSMUSG00000025780 | 09378 | .0231403 | .0108586 | .13 |
| Prkrip1 | hr5: 136656226-136674824 | NSMUSG00000039737 | 6801 | .529968 | .248691 | .13 |
| Nkx2-1 | hr12: 57632923-57637895 | NSMUSG00000001496 | 1869 | .175959 | .0825883 | .13 |
| 8430431K14Rik | hr19: 30638976-31839523 | A | A | .0839328 | .0394124 | .13 |
| Gpr84 | hr15: 103138665-103140869 | NSMUSG00000063234 | 0910 | .27271 | .06725 | .13 |
| Olfr1386 | hr11: 49283555-49284683 | NSMUSG00000062285 | 57888 | .13049 | .41269 | .13 |
| Morn2 | hr17: 80637643-80696816 | NSMUSG00000045257 | 78462 | .90297 | .83562 | .13 |
| Dact1 | hr12: 72410870-72421094 | NSMUSG00000044548 | 9036 | .265523 | .124954 | .12 |
| Plxna4 | hr6: 32094558-32538192 | NSMUSG00000029765 | 43743 | .295843 | .139741 | .12 |
| Nsd1 | hr13: 55311142-55419686 | NSMUSG00000021488 | 8193 | .145237 | .0686074 | .12 |
| Stat1 | hr1: 52176281-52218709 | NSMUSG00000026104 | 0846 | .391142 | .185097 | .11 |
| Umodl1 | hr17: 31091627-31147655 | NSMUSG00000054134 | 2020 | .0305321 | .0144497 | .11 |
| Kcnc2 | hr10: 111708178-111903360 | NSMUSG00000035681 | 68345 | .0350485 | .0165902 | .11 |
| Tmcc1 | hr6: 115968635-116143392 | NSMUSG00000030126 | 30401 | .348401 | .164917 | .11 |
| Ext2 | hr2: 93535787-93662725 | NSMUSG00000027198 | 4043 | .91596 | .433621 | .11 |
| 1110004F10Rik | hr7: 123236893-123248724 | NSMUSG00000030663 | 6372 | .320167 | .151639 | .11 |
| Taf4b | hr18: 14941753-15058868 | NSMUSG00000054321 | 2504 | .426325 | .202021 | .11 |
| D10Bwg1379e | hr10: 18307816-18463564 | NSMUSG00000019852 | 15821 | .080692 | .0382478 | .11 |
| Slc40a1 | hr1: 45964914-45982439 | NSMUSG00000025993 | 3945 | .370139 | .175672 | .11 |
| Igf2as | hr7: 149836672-149856261 | NSMUSG00000086266 | A | .119078 | .056539 | .11 |
| Oip5 | hr2: 119435267-119475896 | NSMUSG00000072980 | 0645 | .11563 | .530721 | .10 |
| Plin2 | hr4: 86302468-86315963 | NSMUSG00000028494 | 1520 | .49716 | .66437 | .10 |
| Vapb | hr2: 173563071-173609837 | NSMUSG00000054455 | 6491 | .81393 | .33998 | .10 |
| Tram1 | hr1: 13554782-13579945 | NSMUSG00000025935 | 2265 | .22909 | .58621 | .10 |
| Ube2e1 | hr14: 19103655-19164358 | NSMUSG00000021774 | 00503235 | .422949 | .201788 | .10 |
| Rgs12 | hr5: 35292096-35376242 | NSMUSG00000029101 | 1729 | .116335 | .055526 | .10 |
| Dnahc17 | hr11: 117848170-117990533 | NSMUSG00000033987 | 9926 | .0229251 | .0109431 | .09 |
| Itpr2 | hr6: 146056820-146450745 | NSMUSG00000030287 | 6439 | .147078 | .0702586 | .09 |
| Rhoq | hr17: 87362450-87424741 | NSMUSG00000024143 | 04215 | .35796 | .171001 | .09 |
| Galnt13 | hr2: 54288797-54970155 | NSMUSG00000060988 | 71786 | .0692642 | .0331018 | .09 |
| Pappa2 | hr1: 160641861-160887569 | NSMUSG00000073530 | 3850 | .0616862 | .0294867 | .09 |
| Chd6 | hr2: 160772713-160934792 | NSMUSG00000057133 | 1389 | .133363 | .0638031 | .09 |
| Bach2 | hr4: 32504409-32673083 | NSMUSG00000040270 | 2014 | .0547348 | .0262645 | .08 |
| C130021I20Rik | hr2: 33496712-33501183 | NSMUSG00000052951 | A | .221073 | .106083 | .08 |
| Peg3 | hr7: 6658670-6683130 | NSMUSG00000002265 | 8616 | .240511 | .115643 | .08 |
| Tm7sf3 | hr6: 146550797-146583114 | NSMUSG00000040234 | 7623 | .125893 | .0606533 | .08 |
| Clcc1 | hr3: 108456830-108525217 | NSMUSG00000027884 | 29725 | .30627 | .629682 | .07 |
| Olfr1170 | hr2: 88064236-88065187 | NSMUSG00000075133 | 58525 | .31225 | .04461 | .07 |
| Slc15a3 | hr19: 10917033-10944269 | NSMUSG00000024737 | 5221 | .754894 | .364272 | .07 |
| 4930455F23Rik | hr1: 166205728-166217978 | NSMUSG00000026578 | 4895 | .0795118 | .0383705 | .07 |
| Inpp5j | hr11: 3394274-3404824 | NSMUSG00000034570 | 70835 | .651931 | .315275 | .07 |
| Prkag1 | hr15: 98643227-98661939 | NSMUSG00000067713 | 9082 | .94901 | .942796 | .07 |
| Nck2 | hr1: 43502595-43627363 | NSMUSG00000066877 | 7974 | .2352 | .598526 | .06 |
| Loxl3 | hr6: 82984217-83004565 | NSMUSG00000000693 | 6950 | .0859827 | .0416956 | .06 |
| Klri2 | hr6: 129679058-129690502 | NSMUSG00000043932 | 20407 | .214297 | .104012 | .06 |
| Pls3 | hrX: 73030992-73120509 | NSMUSG00000016382 | 02866 | .395616 | .192044 | .06 |
| Nedd4l | hr18: 65047409-65377480 | NSMUSG00000024589 | 3814 | .0554148 | .0269018 | .06 |
| Dpp10 | hr1: 125228714-125942136 | NSMUSG00000036815 | 69109 | .138539 | .0673557 | .06 |
| Rgl3 | hr9: 21775970-21793897 | NSMUSG00000040146 | 1746 | .0818267 | .0398715 | .05 |
| Mtss1 | hr15: 58772788-58913581 | NSMUSG00000022353 | 11401 | .84025 | .897004 | .05 |
| 5031425F14Rik | hr2: 166272950-166284270 | NSMUSG00000085129 | A | .365918 | .17837 | .05 |
| Bfsp1 | hr2: 143652263-143688909 | NSMUSG00000027420 | 2075 | .0739791 | .0361219 | .05 |
| Atp8b1 | hr18: 64688632-64820654 | NSMUSG00000039529 | 4670 | .0896076 | .043835 | .04 |
| Fpgs | hr2: 32502114-32549695 | NSMUSG00000009566 | 4287 | .202461 | .0990999 | .04 |
| Fam125a | hr8: 74066828-74071925 | NSMUSG00000031813 | 3711 | .31919 | .647507 | .04 |
| B3gat1 | hr9: 26559146-26568923 | NSMUSG00000045994 | 6898 | .272951 | .134187 | .03 |
| Mir182 | hr6: 30115917-30115992 | NSMUSG00000076361 | A | 04.317 | 46.534 | .03 |
| Abt1 | hr13: 23510229-23515735 | NSMUSG00000036376 | 0946 | .159538 | .078579 | .03 |
| Prex2 | hr1: 10983545-11293763 | NSMUSG00000048960 | 09294 | .0365738 | .018031 | .03 |
| Lrp2 | hr2: 69262391-69424124 | NSMUSG00000027070 | 4725 | .0352567 | .0174075 | .03 |
| S100pbp | hr4: 128828068-128866726 | NSMUSG00000040928 | 4648 | .00735 | .498044 | .02 |
| Fam198b | hr3: 79689851-79750200 | NSMUSG00000027955 | 8659 | .195053 | .0966113 | .02 |
| Eif4e2 | hr1: 89110488-89137063 | NSMUSG00000026254 | 6987 | .61501 | .800017 | .02 |
| Den | hr10: 96942133-96980796 | NSMUSG00000019929 | 3179 | .121843 | .0603649 | .02 |
| Slc34a2 | hr5: 53440591-53462902 | NSMUSG00000029188 | 0531 | .10451 | .0517922 | .02 |
| Pgm5 | hr19: 24748497-24936332 | NSMUSG00000041731 | 26041 | .0223646 | .0110897 | .02 |
| NA | hr8: 124785622-124786185 | A | A | .79188 | .38467 | .02 |
| NA | hr17: 74585635-74587473 | A | A | .61663 | .305826 | .02 |
| Utp14a | hrX: 45610110-45638603 | NSMUSG00000063785 | 2554 | .762063 | .37863 | .01 |
| Fahd1 | hr17: 24985840-24987247 | NSMUSG00000045316 | 8636 | .271261 | .134916 | .01 |
| Tmem246 | hr4: 49597377-49610742 | NSMUSG00000039611 | 7063 | .81312 | .40011 | .01 |
| Arfgap3 | hr15: 83130169-83180677 | NSMUSG00000054277 | 6251 | .416481 | .20749 | .01 |
| 1700012B15Rik | hr12: 3235790-3309969 | NSMUSG00000079179 | A | .841304 | .420163 | .00 |
| Lmbrd1 | hr1: 24685382-24823146 | NSMUSG00000073725 | 8421 | .0406311 | .0203128 | .00 |

While the majority of the transcripts detected encode proteins, noncoding RNAs were also present (FIG. 30D).

Small nucleolar RNAs and various subunits of histone H1 were robustly represented in unstimulated exosomes, while reads that mapped to miRNAs were more abundant after LPS stimulation. Upon further investigation of sequences that encode LPS-responsive miRNAs and those altered in patients with CRPS, at least three miRNAs (let-7b, let-7c and mmu-miR-24) were present as both pre-miRNA and mature miRNA forms.

### LPS Stimulation Leads to Increased Exosomal Cytokines

Pathway analysis of exosomal RNAs from LPS-stimulated cells compared to total transcripts detected revealed perturbations in multiple cellular pathways (Table 7).

TABLE 7
Pathway analysis of exosomal RNAs from LPS-stimulated cells compared with
naive cells

| | | | | fold | |
|---|---|---|---|---|---|
| Pathway | reference | observed | expected | enrichment | sig |
| REACTOME RNA POL I PROMOTER | 1 | 1 | .57563 | .66980 | |
| OPENING | | | | | |
| REACTOME PACKAGING OF | 7 | 5 | .22679 | .74764 | |
| TELOMERE ENDS | | | | | |
| REACTOME AMYLOIDS | 7 | 1 | .09890 | .56301 | |
| MIPS HISTONE H31 COMPLEX | 5 | | .30816 | .87990 | .75E−07 |
| REACTOME RNA POL I | 3 | 5 | .49426 | .37028 | |
| TRANSCRIPTION | | | | | |
| MIPS EMERIN COMPLEX 24 | 0 | 1 | .74421 | .30658 | .70E−07 |
| REACTOME MEIOTIC | 6 | 6 | .75590 | .25446 | |
| RECOMBINATION | | | | | |
| REACTOME DEPOSITION OF NEW | 6 | 3 | .01169 | .73325 | .08E−13 |
| CENPA CONTAINING NUCLEOSOMES | | | | | |
| AT THE CENTROMERE | | | | | |
| REACTOME MTORC1 MEDIATED | 0 | | .87211 | .73325 | .00086805 |
| SIGNALLING | | | | | |
| SA REG CASCADE OF CYCLIN EXPR | 0 | | .87211 | .73325 | .00086805 |
| REACTOME MEIOTIC SYNAPSIS | 4 | 6 | .70937 | .52091 | .79E−14 |
| KEGG SYSTEMIC LUPUS | 4 | 0 | .32569 | .46024 | |
| ERYTHEMATOSUS | | | | | |
| REACTOME TELOMERE | 4 | 8 | .58147 | .01660 | .26E−14 |
| MAINTENANCE | | | | | |
| REACTOME MEIOSIS | 6 | 7 | .50011 | .93326 | |
| REACTOME RNA POL I RNA POL III | 1 | 9 | .93616 | .91422 | |
| AND MITOCHONDRIAL | | | | | |
| TRANSCRIPTION | | | | | |
| REACTOME REGULATION OF IFNA | 0 | | .87211 | .58660 | .00787408 |
| SIGNALING | | | | | |
| REACTOME REGULATION OF KIT | 3 | | .13374 | .41019 | .00354957 |
| SIGNALING | | | | | |
| SA G1 AND S PHASES | 3 | | .13374 | .41019 | .00354957 |
| MIPS DGCR8 MULTIPROTEIN | | | .69768 | .29994 | .02653312 |
| COMPLEX | | | | | |
| BIOCARTA IGF1MTOR PATHWAY | 9 | | .65700 | .22450 | .00074325 |
| REACTOME ALPHA LINOLENIC ACID | 1 | | .95932 | .16964 | .01152928 |
| ALA METABOLISM | | | | | |
| REACTOME APOPTOSIS INDUCED | 1 | | .95932 | .16964 | .01152928 |
| DNA FRAGMENTATION | | | | | |
| REACTOME ACTIVATION OF BH3 | 4 | | .22095 | .09518 | .00512911 |
| ONLY PROTEINS | | | | | |
| MIPS PA700 COMPLEX | 0 | | .74421 | .01328 | .00105807 |
| MIPS EMERIN COMPLEX 32 | 6 | | .26747 | .96917 | .00022552 |
| MIPS 26S PROTEASOME | 1 | | .83142 | .82217 | .00146875 |
| REACTOME TRANSFERRIN | 8 | | .56979 | .82217 | .00322194 |
| ENDOCYTOSIS AND RECYCLING | | | | | |
| BIOCARTA CARM1 PATHWAY | 2 | | .04653 | .82217 | .01611796 |
| MIPS 12S U11 SNRNP | 2 | | .04653 | .82217 | .01611796 |
| REACTOME AKT PHOSPHORYLATES | 2 | | .04653 | .82217 | .01611796 |
| TARGETS IN THE CYTOSOL | | | | | |
| BIOCARTA CACAM PATHWAY | | | .78489 | .82217 | .03726385 |
| BIOCARTA LYM PATHWAY | | | .78489 | .82217 | .03726385 |
| BIOCARTA PLCE PATHWAY | | | .78489 | .82217 | .03726385 |
| BIOCARTA RANMS PATHWAY | | | .78489 | .82217 | .03726385 |
| REACTOME ACTIVATION OF | | | .78489 | .82217 | .03726385 |
| CHAPERONE GENES BY ATF6 ALPHA | | | | | |
| REACTOME CHYLOMICRON | | | .78489 | .82217 | .03726385 |
| MEDIATED LIPID TRANSPORT | | | | | |
| REACTOME PURINE | | | .78489 | .82217 | .03726385 |
| RIBONUCLEOSIDE MONOPHOSPHATE | | | | | |
| BIOSYNTHESIS | | | | | |
| PID IGF1 PATHWAY | 8 | | .44190 | .68566 | .00042492 |
| REACTOME CHROMOSOME | 7 | 1 | .45942 | .66455 | .44E−11 |
| MAINTENANCE | | | | | |
| BIOCARTA MTOR PATHWAY | 2 | | .91863 | .64843 | .00199377 |
| BIOCARTA ARF PATHWAY | 6 | | .39537 | .58328 | .00966213 |
| PID SMAD2 3PATHWAY | 6 | | .39537 | .58328 | .00966213 |
| REACTOME DESTABILIZATION OF | 6 | | .39537 | .58328 | .00966213 |
| MRNA BY BRF1 | | | | | |
| REACTOME N GLYCAN TRIMMING IN | 3 | | .13374 | .52815 | .0217039 |
| THE ER AND CALNEXIN | | | | | |
| CALRETICULIN CYCLE | | | | | |
| BIOCARTA IL6 PATHWAY | 0 | | .74421 | .43995 | .00578361 |
| PID PDGFRAPATHWAY | 0 | | .74421 | .43995 | .00578361 |
| BIOCARTA CFTR PATHWAY | 0 | | .87211 | .43995 | .04985947 |
| MIPS TNF ALPHA NF KAPPA B | 0 | | .87211 | .43995 | .04985947 |
| SIGNALING COMPLEX 10 | | | | | |
| PID ALK2PATHWAY | 0 | | .87211 | .43995 | .04985947 |
| REACTOME UNWINDING OF DNA | 0 | | .87211 | .43995 | .04985947 |
| BIOCARTA ERK5 PATHWAY | 7 | | .48258 | .37250 | .01272256 |
| REACTOME TRANSCRIPTION | 67 | 9 | 4.56416 | .36442 | .03E−14 |
| MIPS PA700 20S PA28 COMPLEX | 5 | 0 | .05237 | .27614 | .00059296 |
| BIOCARTA HIF PATHWAY | 4 | | .22095 | .27614 | .02833382 |
| BIOCARTA TOB1 PATHWAY | 4 | | .22095 | .27614 | .02833382 |
| PID INTEGRIN5 PATHWAY | 4 | | .22095 | .27614 | .02833382 |
| BIOCARTA G1 PATHWAY | 5 | | .18026 | .21062 | .00445897 |
| BIOCARTA ACTINY PATHWAY | 8 | | .56979 | .18514 | .01637665 |
| BIOCARTA MCM PATHWAY | 8 | | .56979 | .18514 | .01637665 |
| MIPS LARGE DROSHA COMPLEX | 8 | | .56979 | .18514 | .01637665 |
| PID ARF 3PATHWAY | 8 | | .56979 | .18514 | .01637665 |
| REACTOME BASIGIN INTERACTIONS | 8 | | .56979 | .18514 | .01637665 |
| BIOCARTA ALK PATHWAY | 9 | | .52911 | .16317 | .00266064 |
| PID FOXM1PATHWAY | 3 | | .87795 | .12723 | .00159401 |
| BIOCARTA SALMONELLA PATHWAY | 1 | | .95932 | .12723 | .06423329 |
| MIPS MULTISYNTHETASE COMPLEX | 1 | | .95932 | .12723 | .06423329 |
| MIPS TNF ALPHA NF KAPPA B | 1 | | .95932 | .12723 | .06423329 |
| SIGNALING COMPLEX 6 | | | | | |
| REACTOME ACTIVATION OF | 1 | | .95932 | .12723 | .06423329 |
| CHAPERONES BY ATF6 ALPHA | | | | | |
| REACTOME CALNEXIN CALRETICULIN | 1 | | .95932 | .12723 | .06423329 |
| CYCLE | | | | | |
| REACTOME CDC6 ASSOCIATION | 1 | | .95932 | .12723 | .06423329 |
| WITH THE ORC ORIGIN COMPLEX | | | | | |
| REACTOME METABOLISM OF | 1 | | .95932 | .12723 | .06423329 |
| PORPHYRINS | | | | | |
| REACTOME REGULATION OF IFNG | 1 | | .95932 | .12723 | .06423329 |
| SIGNALING | | | | | |
| REACTOME SIGNAL ATTENUATION | 1 | | .95932 | .12723 | .06423329 |
| REACTOME ASSEMBLY OF THE PRE | 0 | 6 | .23263 | .05773 | .79E−05 |
| REPLICATIVE COMPLEX | | | | | |
| BIOCARTA RARRXR PATHWAY | 5 | | .30816 | .05773 | .03603768 |
| BIOCARTA GLEEVEC PATHWAY | 3 | | .00584 | .99126 | .01202209 |
| REACTOME AMINO ACID TRANSPORT | 3 | | .00584 | .99126 | .01202209 |
| ACROSS THE PLASMA MEMBRANE | | | | | |
| REACTOME CYTOSOLIC TRNA | 3 | | .00584 | .99126 | .01202209 |
| AMINOACYLATION | | | | | |
| PID RB 1PATHWAY | 4 | 4 | .70937 | .97280 | .00015926 |
| BIOCARTA PROTEASOME PATHWAY | 7 | | .35468 | .97280 | .00706454 |
| REACTOME CDK MEDIATED | 3 | 1 | .75005 | .93329 | .00089446 |
| PHOSPHORYLATION AND REMOVAL | | | | | |
| OF CDC6 | | | | | |
| REACTOME SPHINGOLIPID DE NOVO | 8 | | .44190 | .86663 | .00872485 |
| BIOSYNTHESIS | | | | | |
| REACTOME INTEGRIN ALPHAIIB | 4 | | .09305 | .86663 | .01487366 |
| BETA3 SIGNALING | | | | | |
| BIOCARTA P53 PATHWAY | 6 | | .39537 | .86663 | .04482953 |
| REACTOME INSULIN RECEPTOR | 6 | | .39537 | .86663 | .04482953 |
| RECYCLING | | | | | |
| REACTOME PYRUVATE METABOLISM | 6 | | .39537 | .86663 | .04482953 |
| BIOCARTA ARENRF2 PATHWAY | 2 | | .04653 | .86663 | .08027173 |
| BIOCARTA DREAM PATHWAY | 2 | | .04653 | .86663 | .08027173 |
| BIOCARTA LAIR PATHWAY | 2 | | .04653 | .86663 | .08027173 |
| BIOCARTA RANKL PATHWAY | 2 | | .04653 | .86663 | .08027173 |
| MIPS TFIID BETA COMPLEX | 2 | | .04653 | .86663 | .08027173 |
| REACTOME ASSOCIATION OF | 2 | | .04653 | .86663 | .08027173 |
| LICENSING FACTORS WITH THE PRE | | | | | |
| REPLICATIVE COMPLEX | | | | | |
| REACTOME PROTEOLYTIC | 2 | | .04653 | .86663 | .08027173 |
| CLEAVAGE OF SNARE COMPLEX | | | | | |
| PROTEINS | | | | | |
| REACTOME ORC1 REMOVAL FROM | 1 | 5 | .31984 | .81963 | .00017859 |
| CHROMATIN | | | | | |
| PID MET PATHWAY | 5 | 8 | .54079 | .75196 | .89E−05 |
| REACTOME SCFSKP2 MEDIATED | 0 | 2 | .36053 | .75196 | .00098036 |
| DEGRADATION OF P27 P21 | | | | | |
| BIOCARTA CREB PATHWAY | 5 | | .18026 | .75196 | .01816251 |
| REACTOME PRE NOTCH | 5 | | .18026 | .75196 | .01816251 |
| TRANSCRIPTION AND TRANSLATION | | | | | |
| REACTOME TRANSPORT OF | 5 | | .18026 | .75196 | .01816251 |
| RIBONUCLEOPROTEINS INTO THE | | | | | |
| HOST NUCLEUS | | | | | |
| PID KITPATHWAY | 6 | 1 | .01169 | .74199 | .00162837 |
| PID IL6 7PATHWAY | 2 | 0 | .66284 | .73012 | .00271295 |
| REACTOME LYSOSOME VESICLE | 1 | | .83142 | .73012 | .03128875 |
| BIOGENESIS | | | | | |
| REACTOME RNA POL I | 1 | | .83142 | .73012 | .03128875 |
| TRANSCRIPTION INITIATION | | | | | |
| REACTOME APC C CDH1 MEDIATED | 9 | 4 | .14542 | .72087 | .00043216 |
| DEGRADATION OF CDC20 AND | | | | | |
| OTHER APC C CDH1 TARGETED | | | | | |
| PROTEINS IN LATE MITOSIS EARLY | | | | | |
| G1 | | | | | |
| PID IFNGPATHWAY | 8 | | .31400 | .71575 | .00453664 |
| KEGG CHRONIC MYELOID LEUKEMIA | 8 | 6 | .93032 | .69800 | .00019146 |
| KEGG DNA REPLICATION | 4 | | .96516 | .69800 | .00762093 |
| REACTOME SIGNALING BY FGFR1 | 7 | | .48258 | .69800 | .0547086 |
| FUSION MUTANTS | | | | | |
| SA PTEN PATHWAY | 7 | | .48258 | .69800 | .0547086 |
| SA TRKA RECEPTOR | 7 | | .48258 | .69800 | .0547086 |
| REACTOME DESTABILIZATION OF | 7 | 1 | .09890 | .68365 | .00196135 |
| MRNA BY AUF1 HNRNP D0 | | | | | |
| PID IL2 PI3KPATHWAY | 0 | | .61632 | .67552 | .01287586 |
| REACTOME G1 PHASE | 0 | | .61632 | .67552 | .01287586 |
| REACTOME CROSS PRESENTATION | 3 | 0 | .75005 | .66663 | .00326401 |
| OF SOLUBLE EXOGENOUS ANTIGENS | | | | | |
| ENDOSOMES | | | | | |
| BIOCARTA RACCYCD PATHWAY | 6 | | .26747 | .64612 | .02191616 |
| REACTOME PIP3 ACTIVATES AKT | 6 | | .26747 | .64612 | .02191616 |
| SIGNALING | | | | | |
| REACTOME PKB MEDIATED EVENTS | 6 | | .26747 | .64612 | .02191616 |
| MIPS 20S PROTEASOME | 3 | | .13374 | .64612 | .09784208 |
| MIPS HES1 PROMOTER NOTCH | 3 | | .13374 | .64612 | .09784208 |
| ENHANCER COMPLEX | | | | | |
| PID CIRCADIANPATHWAY | 3 | | .13374 | .64612 | .09784208 |
| REACTOME ENOS ACTIVATION AND | 3 | | .13374 | .64612 | .09784208 |
| REGULATION | | | | | |
| REACTOME TRAF3 DEPENDENT IRF | 3 | | .13374 | .64612 | .09784208 |
| ACTIVATION PATHWAY | | | | | |
| REACTOME TRANSPORT OF MATURE | 8 | 1 | .18611 | .62774 | .00234764 |
| TRANSCRIPT TO CYTOPLASM | | | | | |
| REACTOME REGULATION OF MITOTIC | 0 | 6 | .10474 | .62092 | .00027366 |
| CELL CYCLE | | | | | |
| PID AURORA B PATHWAY | 5 | | .05237 | .62092 | .00914103 |
| REACTOME AUTODEGRADATION OF | 4 | 0 | .83726 | .60602 | .00390047 |
| THE E3 UBIQUITIN LIGASE COP1 | | | | | |
| REACTOME VIF MEDIATED | 4 | 0 | .83726 | .60602 | .00390047 |
| DEGRADATION OF APOBEC3G | | | | | |
| BIOCARTA RAS PATHWAY | 2 | | .91863 | .60602 | .03767427 |
| REACTOME REGULATION OF MRNA | 5 | 7 | .54079 | .59907 | .0001985 |
| STABILITY BY PROTEINS THAT BIND | | | | | |
| AU RICH ELEMENTS | | | | | |
| KEGG SPHINGOLIPID METABOLISM | 1 | | .70353 | .58921 | .01541141 |
| REACTOME P53 INDEPENDENT G1 S | 5 | 0 | .92447 | .54811 | .00463116 |
| DNA DAMAGE CHECKPOINT | | | | | |
| REACTOME REGULATION OF | 5 | 0 | .92447 | .54811 | .00463116 |
| ORNITHINE DECARBOXYLASE ODC | | | | | |
| PID CD40 PATHWAY | 7 | | .35468 | .54811 | .02615948 |
| PID NCADHERINPATHWAY | 7 | | .35468 | .54811 | .02615948 |
| REACTOME IRON UPTAKE AND | 7 | | .35468 | .54811 | .02615948 |
| TRANSPORT | | | | | |
| BIOCARTA CELLCYCLE PATHWAY | 8 | | .56979 | .54811 | .06566059 |
| BIOCARTA ETS PATHWAY | 8 | | .56979 | .54811 | .06566059 |
| BIOCARTA TGFB PATHWAY | 8 | | .56979 | .54811 | .06566059 |
| BIOCARTA TNFR2 PATHWAY | 8 | | .56979 | .54811 | .06566059 |
| MIPS LARC COMPLEX | 8 | | .56979 | .54811 | .06566059 |
| REACTOME DEADENYLATION OF | 8 | | .56979 | .54811 | .06566059 |
| MRNA | | | | | |
| REACTOME RNA POL I | 8 | | .56979 | .54811 | .06566059 |
| TRANSCRIPTION TERMINATION | | | | | |
| PID MYC ACTIVPATHWAY | 8 | 5 | .93032 | .52938 | .00062962 |
| REACTOME APC C CDC20 MEDIATED | 9 | 3 | .14542 | .52652 | .00143617 |
| DEGRADATION OF MITOTIC | | | | | |
| PROTEINS | | | | | |
| REACTOME CYCLIN E ASSOCIATED | 9 | 3 | .14542 | .52652 | .00143617 |
| EVENTS DURING G1 S TRANSITION | | | | | |
| REACTOME P53 DEPENDENT G1 DNA | 0 | 1 | .36053 | .52263 | .0033046 |
| DAMAGE RESPONSE | | | | | |
| KEGG PROTEASOME | 1 | | .57563 | .51704 | .00769946 |
| PID PI3KPLCTRKPATHWAY | 2 | | .79074 | .50830 | .01828226 |
| REACTOME S PHASE | 01 | 2 | .80827 | .49765 | .66E−05 |
| REACTOME SCF BETA TRCP | 6 | 0 | .01169 | .49272 | .00546527 |
| MEDIATED DEGRADATION OF EMI1 | | | | | |
| REACTOME RNA POL III | 3 | | .00584 | .49272 | .04479988 |
| TRANSCRIPTION INITIATION FROM | | | | | |
| TYPE 3 PROMOTER | | | | | |
| REACTOME M G1 TRANSITION | 4 | 6 | .45358 | .47924 | .00053262 |
| REACTOME CDT1 ASSOCIATION WITH | 1 | 1 | .44774 | .47317 | .00388833 |
| THE CDC6 ORC ORIGIN COMPLEX | | | | | |
| REACTOME SPHINGOLIPID | 1 | 1 | .44774 | .47317 | .00388833 |
| METABOLISM | | | | | |
| REACTOME FACTORS INVOLVED IN | 8 | 1 | .54663 | .45711 | .84E−05 |
| MEGAKARYOCYTE DEVELOPMENT | | | | | |
| AND PLATELET PRODUCTION | | | | | |
| MIPS CDC5L COMPLEX | 8 | | .44190 | .45711 | .03091438 |
| REACTOME PLATELET | 8 | | .44190 | .45711 | .03091438 |
| AGGREGATION PLUG FORMATION | | | | | |
| BIOCARTA CARM ER PATHWAY | 3 | | .87795 | .43229 | .02150853 |
| BIOCARTA IL2RB PATHWAY | 3 | | .87795 | .43229 | .02150853 |
| REACTOME SYNTHESIS OF DNA | 5 | 8 | .41290 | .42820 | .00032191 |
| PID ECADHERIN KERATINOCYTE | 9 | | .65700 | .41400 | .0776591 |
| PATHWAY | | | | | |
| PID EPHA2 FWDPATHWAY | 9 | | .65700 | .41400 | .0776591 |
| REACTOME APC CDC20 MEDIATED | 9 | | .65700 | .41400 | .0776591 |
| DEGRADATION OF NEK2A | | | | | |
| PID LYMPHANGIOGENESIS PATHWAY | 4 | | .09305 | .38885 | .0526752 |
| REACTOME AUTODEGRADATION OF | 3 | 1 | .62216 | .37984 | .00530123 |
| CDH1 BY CDH1 APC C | | | | | |
| PID MYC REPRESSPATHWAY | 8 | 2 | .05821 | .37238 | .00376613 |
| REACTOME SIGNALING BY WNT | 8 | 2 | .05821 | .37238 | .00376613 |
| REACTOME CD28 CO STIMULATION | 9 | | .52911 | .37238 | .0361996 |
| REACTOME DNA STRAND | 9 | | .52911 | .37238 | .0361996 |
| ELONGATION | | | | | |
| REACTOME INTERACTIONS OF VPR | 9 | | .52911 | .37238 | .0361996 |
| WITH HOST CELLULAR PROTEINS | | | | | |
| REACTOME MRNA 3 END | 9 | | .52911 | .37238 | .0361996 |
| PROCESSING | | | | | |
| REACTOME RNA POL III | 9 | | .52911 | .37238 | .0361996 |
| TRANSCRIPTION | | | | | |
| PID | 4 | | .96516 | .36075 | .02510902 |
| P38ALPHABETADOWNSTREAMPATHWAY | | | | | |
| REACTOME ACTIVATION OF THE | 9 | 0 | .27332 | .34010 | .00868413 |
| MRNA UPON BINDING OF THE CAP | | | | | |
| BINDING COMPLEX AND EIFS AND | | | | | |
| SUBSEQUENT BINDING TO 43S | | | | | |
| REACTOME ER PHAGOSOME | 5 | 1 | .79658 | .29330 | .00709113 |
| PATHWAY | | | | | |
| KEGG N GLYCAN BIOSYNTHESIS | 0 | | .48842 | .29330 | .02029636 |
| PID INSULIN PATHWAY | 0 | | .48842 | .29330 | .02029636 |
| REACTOME DEADENYLATION | 0 | | .48842 | .29330 | .02029636 |
| DEPENDENT MRNA DECAY | | | | | |
| REACTOME PI3K CASCADE | 0 | | .48842 | .29330 | .02029636 |
| REACTOME GAB1 SIGNALOSOME | 5 | | .05237 | .29330 | .02910102 |
| MIPS 28S RIBOSOMAL SUBUNIT | 5 | | .18026 | .29330 | .06130337 |
| MITOCHONDRIAL | | | | | |
| MIPS 40S RIBOSOMAL SUBUNIT | 5 | | .18026 | .29330 | .06130337 |
| CYTOPLASMIC | | | | | |
| REACTOME INTRINSIC PATHWAY | 5 | | .18026 | .29330 | .06130337 |
| FOR APOPTOSIS | | | | | |
| REACTOME NUCLEAR EVENTS | 5 | | .18026 | .29330 | .06130337 |
| KINASE AND TRANSCRIPTION | | | | | |
| FACTOR ACTIVATION | | | | | |
| BIOCARTA G2 PATHWAY | 0 | | .74421 | .29330 | .09066712 |
| BIOCARTA INSULIN PATHWAY | 0 | | .74421 | .29330 | .09066712 |
| BIOCARTA RAC1 PATHWAY | 0 | | .74421 | .29330 | .09066712 |
| REACTOME CD28 DEPENDENT PI3K | 0 | | .74421 | .29330 | .09066712 |
| AKT SIGNALING | | | | | |
| REACTOME CTLA4 INHIBITORY | 0 | | .74421 | .29330 | .09066712 |
| SIGNALING | | | | | |
| REACTOME G BETA GAMMA | 0 | | .74421 | .29330 | .09066712 |
| SIGNALLING THROUGH PI3KGAMMA | | | | | |
| KEGG PANCREATIC CANCER | 6 | 3 | .75590 | .25855 | .00410487 |
| REACTOME METABOLISM OF NON | 1 | | .57563 | .23737 | .02335016 |
| CODING RNA | | | | | |
| BIOCARTA P38MAPK PATHWAY | 6 | | .13958 | .22960 | .03350014 |
| PID NFAT TFPATHWAY | 6 | | .13958 | .22960 | .03350014 |
| REACTOME CLEAVAGE OF GROWING | 6 | | .13958 | .22960 | .03350014 |
| TRANSCRIPT IN THE TERMINATION | | | | | |
| REGION | | | | | |
| REACTOME PYRUVATE METABOLISM | 6 | | .13958 | .22960 | .03350014 |
| AND CITRIC ACID TCA CYCLE | | | | | |
| REACTOME CELL CYCLE | 55 | 9 | 0.95974 | .22870 | .40E−10 |
| KEGG SNARE INTERACTIONS IN | 1 | | .70353 | .21932 | .04841913 |
| VESICULAR TRANSPORT | | | | | |
| PID INTEGRIN A4B1 PATHWAY | 1 | | .70353 | .21932 | .04841913 |
| REACTOME MAPK TARGETS | 1 | | .70353 | .21932 | .04841913 |
| NUCLEAR EVENTS MEDIATED BY | | | | | |
| MAP KINASES | | | | | |
| REACTOME DOWNSTREAM | 8 | 7 | .67453 | .21512 | .00139246 |
| SIGNALING EVENTS OF B CELL | | | | | |
| RECEPTOR BCR | | | | | |
| REACTOME ANTIVIRAL MECHANISM | 7 | 1 | .97100 | .21283 | .00932075 |
| BY IFN STIMULATED GENES | | | | | |
| REACTOME CELL CYCLE | 04 | 0 | .06990 | .20510 | .00058503 |
| CHECKPOINTS | | | | | |
| REACTOME PI3K CASCADE | 2 | 0 | .53495 | .20510 | .01318753 |
| REACTOME MITOTIC G1 G1 S | 20 | 3 | 0.46527 | .19775 | .00024733 |
| PHASES | | | | | |
| REACTOME SIGNALING BY SCF KIT | 8 | 3 | .93032 | .19213 | .00535402 |
| PID ANGIOPOIETINRECEPTOR | 2 | | .66284 | .18410 | .02671268 |
| PATHWAY | | | | | |
| PID FASPATHWAY | 7 | | .22679 | .16934 | .03832015 |
| PID RET PATHWAY | 7 | | .22679 | .16934 | .03832015 |
| REACTOME REGULATION OF | 3 | 0 | .62216 | .16349 | .01502155 |
| APOPTOSIS | | | | | |
| PID MAPKTRKPATHWAY | 2 | | .79074 | .14997 | .05537379 |
| PID PI3KCIAKTPATHWAY | 2 | | .79074 | .14997 | .05537379 |
| REACTOME PI3K EVENTS IN ERBB4 | 2 | | .79074 | .14997 | .05537379 |
| SIGNALING | | | | | |
| ST GA13 PATHWAY | 2 | | .79074 | .14997 | .05537379 |
| REACTOME HOST INTERACTIONS OF | 07 | 0 | .33153 | .14327 | .00085235 |
| HIV FACTORS | | | | | |
| KEGG COLORECTAL CANCER | 9 | 1 | .14542 | .13782 | .0120553 |
| PID ERBB1 DOWNSTREAM PATHWAY | 7 | 8 | .45942 | .12780 | .00165017 |
| BIOCARTA AT1R PATHWAY | 7 | | .35468 | .12343 | .08079986 |
| PID RAS PATHWAY | 7 | | .35468 | .12343 | .08079986 |
| REACTOME PI3K EVENTS IN ERBB2 | 8 | | .31400 | .11225 | .0435729 |
| SIGNALING | | | | | |
| KEGG NON SMALL CELL LUNG | 9 | | .27332 | .10609 | .02416523 |
| CANCER | | | | | |
| REACTOME PROCESSING OF | 20 | 2 | 0.46527 | .10219 | .00063341 |
| CAPPED INTRON CONTAINING PRE | | | | | |
| MRNA | | | | | |
| REACTOME SIGNALING BY INSULIN | 2 | 5 | .15126 | .09753 | .00447519 |
| RECEPTOR | | | | | |
| MIPS SPLICEOSOME | 26 | 3 | 0.98853 | .09309 | .00051383 |
| PID CERAMIDE PATHWAY | 4 | | .83726 | .08482 | .0344154 |
| REACTOME G1 S TRANSITION | 00 | 8 | .72105 | .06397 | .00234908 |
| PID ILK PATHWAY | 9 | | .40121 | .05809 | .0492682 |
| MIPS 17S U2 SNRNP | 8 | | .44190 | .04759 | .09164453 |
| PID FRA PATHWAY | 8 | | .44190 | .04759 | .09164453 |
| ST WNT BETA CATENIN PATHWAY | 8 | | .44190 | .04759 | .09164453 |
| REACTOME METABOLISM OF RNA | 13 | 8 | 8.57585 | .04567 | .60E−05 |
| PID SMAD2 3NUCLEARPATHWAY | 3 | 3 | .36637 | .04198 | .00982709 |
| MIPS CEN COMPLEX | 4 | | .96516 | .02350 | .07099733 |
| KEGG SPLICEOSOME | 08 | 9 | .41874 | .01726 | .00236924 |
| KEGG NOTCH SIGNALING PATHWAY | 0 | | .48842 | .00664 | .05541374 |
| REACTOME APOPTOSIS | 26 | 2 | 0.98853 | .00209 | .00124253 |
| REACTOME UNFOLDED PROTEIN | 9 | 2 | .01753 | .99417 | .01537787 |
| RESPONSE | | | | | |
| REACTOME DNA REPLICATION | 79 | 1 | 5.61069 | .98582 | .00016443 |
| REACTOME ACTIVATION OF NF | 8 | 0 | .05821 | .97698 | .02713675 |
| KAPPAB IN B CELLS | | | | | |
| REACTOME MRNA SPLICING | 3 | 6 | .11058 | .97273 | .00629565 |
| PID BCR 5PATHWAY | 4 | 1 | .58147 | .97081 | .02153633 |
| REACTOME METABOLISM OF MRNA | 75 | 0 | 5.26185 | .96569 | .00025005 |
| REACTOME PI3K AKT ACTIVATION | 5 | | .05237 | .96569 | .07966554 |
| REACTOME ACTIVATION OF | 1 | | .57563 | .95770 | .06201507 |
| CHAPERONE GENES BY XBP1S | | | | | |
| REACTOME FORMATION OF THE | 1 | | .57563 | .95770 | .06201507 |
| TERNARY COMPLEX AND | | | | | |
| SUBSEQUENTLY THE 43S COMPLEX | | | | | |
| REACTOME TRNA AMINOACYLATION | 1 | | .57563 | .95770 | .06201507 |
| KEGG MTOR SIGNALING PATHWAY | 7 | | .09890 | .95175 | .0485775 |
| KEGG CELL CYCLE | 12 | 9 | .76758 | .94521 | .00361528 |
| REACTOME SIGNALING BY THE B | 12 | 9 | .76758 | .94521 | .00361528 |
| CELL RECEPTOR BCR | | | | | |
| MIPS NOP56P ASSOCIATED PRE | 7 | 3 | .71521 | .93590 | .01516404 |
| RRNA COMPLEX | | | | | |
| REACTOME MITOTIC M M G1 PHASES | 61 | 7 | 4.04090 | .92295 | .00070912 |
| REACTOME MRNA PROCESSING | 38 | 3 | 2.03506 | .91108 | .00185037 |
| REACTOME INTERFERON SIGNALING | 20 | 0 | 0.46527 | .91108 | .00355037 |
| MIPS C COMPLEX SPLICEOSOME | 2 | 2 | .27916 | .91108 | .02109267 |
| PID E2F PATHWAY | 6 | 1 | .75590 | .91108 | .02655764 |
| PID VEGFR1 2 PATHWAY | 6 | 1 | .75590 | .91108 | .02655764 |
| KEGG MELANOMA | 4 | | .70937 | .91108 | .04247646 |
| REACTOME AMINO ACID AND | 6 | | .13958 | .91108 | .08889862 |
| OLIGOPEPTIDE SLC TRANSPORTERS | | | | | |
| REACTOME MRNA SPLICING MINOR | 6 | | .13958 | .91108 | .08889862 |
| PATHWAY | | | | | |
| REACTOME ASPARAGINE N LINKED | 3 | 2 | .36637 | .88490 | .02330884 |
| GLYCOSYLATION | | | | | |
| REACTOME ANTIGEN PROCESSING | 7 | 1 | .84311 | .88256 | .02936261 |
| CROSS PRESENTATION | | | | | |
| REACTOME INSULIN RECEPTOR | 7 | 1 | .84311 | .88256 | .02936261 |
| SIGNALLING CASCADE | | | | | |
| BIOCARTA HIVNEF PATHWAY | 5 | | .79658 | .87634 | .04702087 |
| REACTOME COSTIMULATION BY THE | 9 | | .27332 | .87208 | .05985566 |
| CD28 FAMILY | | | | | |
| REACTOME MAP KINASE ACTIVATION | 9 | | .27332 | .87208 | .05985566 |
| IN TLR CASCADE | | | | | |
| ST JNK MAPK PATHWAY | 7 | | .22679 | .85943 | .098688 |
| KEGG GLIOMA | 6 | | .88379 | .84283 | .0518766 |
| BIOCARTA MAPK PATHWAY | 1 | 3 | .06405 | .84030 | .02247002 |
| PID TGFBRPATHWAY | 0 | | .36053 | .83464 | .06606277 |
| PID CASPASE PATHWAY | 4 | | .83726 | .82422 | .08457654 |
| PID TNFPATHWAY | 4 | | .83726 | .82422 | .08457654 |
| REACTOME APOPTOTIC EXECUTION | 4 | | .83726 | .82422 | .08457654 |
| PHASE | | | | | |
| REACTOME HIV INFECTION | 71 | 7 | 4.91300 | .81050 | .00178834 |
| KEGG JAK STAT SIGNALING | 5 | 5 | .28500 | .81050 | .0171803 |
| PATHWAY | | | | | |
| PID P53REGULATIONPATHWAY | 1 | | .44774 | .79867 | .07265278 |
| KEGG ADHERENS JUNCTION | 4 | 0 | .58147 | .79164 | .04932891 |
| REACTOME TRANSLATION | 16 | 8 | 0.11642 | .77928 | .01149083 |
| REACTOME TRANSCRIPTIONAL | 5 | 0 | .66869 | .76408 | .05394301 |
| REGULATION OF WHITE ADIPOCYTE | | | | | |
| DIFFERENTIATION | | | | | |
| KEGG PROSTATE CANCER | 9 | 2 | .88963 | .74175 | .04032204 |
| REACTOME CELL CYCLE MITOTIC | 84 | 3 | 4.76779 | .73613 | .00024748 |
| REACTOME SRP DEPENDENT | 1 | 2 | .06405 | .69874 | .04754586 |
| COTRANSLATIONAL PROTEIN | | | | | |
| TARGETING TO MEMBRANE | | | | | |
| KEGG P53 SIGNALING PATHWAY | 4 | | .70937 | .69874 | .09472588 |
| PID TRKRPATHWAY | 1 | | .31984 | .69178 | .0809851 |
| REACTOME CELL SURFACE | 1 | | .31984 | .69178 | .0809851 |
| INTERACTIONS AT THE VASCULAR | | | | | |
| WALL | | | | | |
| PID MTOR 4PATHWAY | 2 | | .40705 | .66449 | .08779032 |
| REACTOME TRAF6 MEDIATED | 9 | 0 | .01753 | .66181 | .07519608 |
| INDUCTION OF NFKB AND MAP | | | | | |
| KINASES UPON TLR7 8 OR 9 | | | | | |
| ACTIVATION | | | | | |
| ST INTEGRIN SIGNALING PATHWAY | 9 | 0 | .01753 | .66181 | .07519608 |
| KEGG NEUROTROPHIN SIGNALING | 12 | 6 | .76758 | .63807 | .03345554 |
| PATHWAY | | | | | |
| PID P73PATHWAY | 1 | 0 | .19195 | .61500 | .08753645 |
| REACTOME TCA CYCLE AND | 4 | 3 | .19779 | .58579 | .06404615 |
| RESPIRATORY ELECTRON | | | | | |
| TRANSPORT | | | | | |
| KEGG FC GAMMA R MEDIATED | 7 | 2 | .58732 | .58159 | .07437758 |
| PHAGOCYTOSIS | | | | | |
| REACTOME 3 UTR MEDIATED | 0 | 1 | .97684 | .57664 | .08656957 |
| TRANSLATIONAL REGULATION | | | | | |
| REACTOME RNA POL II | 8 | 2 | .67453 | .56361 | .07963335 |
| TRANSCRIPTION | | | | | |
| REACTOME CYTOKINE SIGNALING IN | 13 | 9 | 8.57585 | .56117 | .01071614 |
| IMMUNE SYSTEM | | | | | |
| REACTOME NGF SIGNALLING VIA | 25 | 7 | 0.90132 | .55944 | .043701 |
| TRKA FROM THE PLASMA | | | | | |
| MEMBRANE | | | | | |
| REACTOME TOLL RECEPTOR | 03 | 4 | .98269 | .55855 | .0632108 |
| CASCADES | | | | | |
| PID PDGFRBPATHWAY | 19 | 6 | 0.37805 | .54171 | .05376787 |
| REACTOME ANTIGEN PROCESSING | 72 | 3 | 5.00021 | .53331 | .02577599 |
| UBIQUITINATION PROTEASOME | | | | | |
| DEGRADATION | | | | | |
| REACTOME IMMUNE SYSTEM | 92 | 2 | 0.34970 | .52445 | .47E−05 |
| REACTOME HIV LIFE CYCLE | 9 | 3 | .63384 | .50570 | .08867646 |
| REACTOME SIGNALING BY FGFR IN | 01 | 3 | .80827 | .47589 | .09998131 |
| DISEASE | | | | | |
| REACTOME PHOSPHOLIPID | 56 | 0 | 3.60485 | .47006 | .05200509 |
| METABOLISM | | | | | |
| REACTOME ADAPTIVE IMMUNE | 25 | 2 | 7.06448 | .40296 | .00780592 |
| SYSTEM | | | | | |
| REACTOME CLASS I MHC MEDIATED | 07 | 5 | 8.05258 | .38484 | .05969153 |
| ANTIGEN PROCESSING | | | | | |
| PRESENTATION | | | | | |
| REACTOME HEMOSTASIS | 53 | 2 | 0.78532 | .36429 | .02389701 |
| KEGG PATHWAYS IN CANCER | 71 | 2 | 3.63406 | .35398 | .04812098 |
| REACTOME TRANSMEMBRANE | 07 | 5 | 6.77364 | .30726 | .06106853 |
| TRANSPORT OF SMALL MOLECULES | | | | | |
| REACTOME METABOLISM OF LIPIDS | 71 | 2 | 2.35511 | .29809 | .04792547 |
| AND LIPOPROTEINS | | | | | |
| REACTOME METABOLISM OF | 36 | 8 | 9.30274 | .29681 | .05825339 |
| PROTEINS | | | | | |

Cellular pathways that are altered by changes in exosomal content after LPS stimulation of cells are listed by fold change and significance.

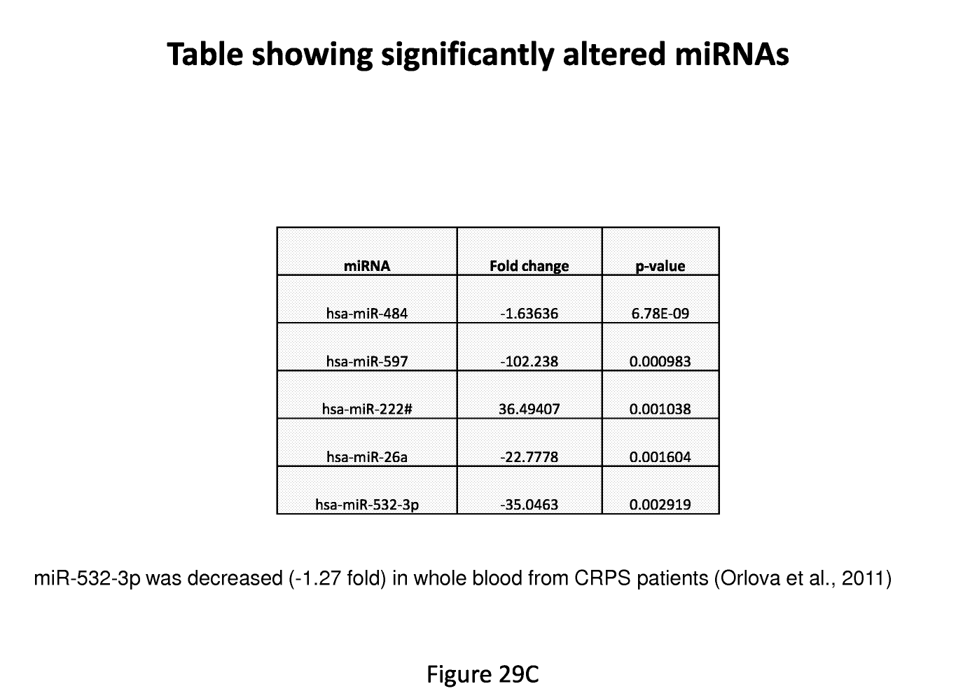

The reactome database was used to analyze the gene ontology of total transcripts. A gene ontology pie chart shows the distribution of exosomal mRNAs categorized by cellular function based on global reactome pathways (FIG. 31). Of the 15883 genes, 5445 are represented. The top 3 pathways that were represented by exosomal transcripts involve signaling, metabolism, and the immune system (FIG. 31). Significantly altered inflammatory pathways representing the innate and adaptive immune systems are shown in Table 8.

TABLE 8
Immune pathways that are significantly altered after LPS treatment

| | Transcripts | | Fold | P |
|---|---|---|---|---|
| Pathway | Total | Altered | change | value |
| Adaptive immune system | 425 | 52 | 1.40 | 0.0078 |
| Class 1 MHC-mediated antigen- | 207 | 25 | 1.38 | 0.0596 |
| processing presentation | | | | |
| Antigen-processing cross-presentation | 67 | 11 | 1.88 | 0.0293 |
| Downstream signaling events of | 88 | 17 | 2.22 | 0.0013 |
| B-cell receptor | | | | |
| Activation of NF-κB in B cells | 58 | 10 | 1.98 | 0.0271 |
| Antigen processing ubiquitination | 172 | 23 | 1.53 | 0.0257 |
| proteosome degradation | | | | |
| Innate immune system | 171 | 19 | 1.27 | 0.1631 |
| Toll receptor cascades | 103 | 14 | 1.56 | 0.0632 |
| MAPK activation in TLR cascade | 49 | 8 | 1.87 | 0.0598 |
| TRAF6-mediated induction of NF- | 69 | 10 | 1.66 | 0.0751 |
| κB & MAPK upon TLR7/8 activation | | | | |
| Cytokine signaling in immune system | 213 | 29 | 1.56 | 0.0107 |

Exosomal RNA datasets from naive and LPS-treated samples were compared and significantly altered pathways involved in immune response are shown with corresponding fold change and P values.
Global pathways are shown in bold.
TRAF = TNF receptor associated factor.

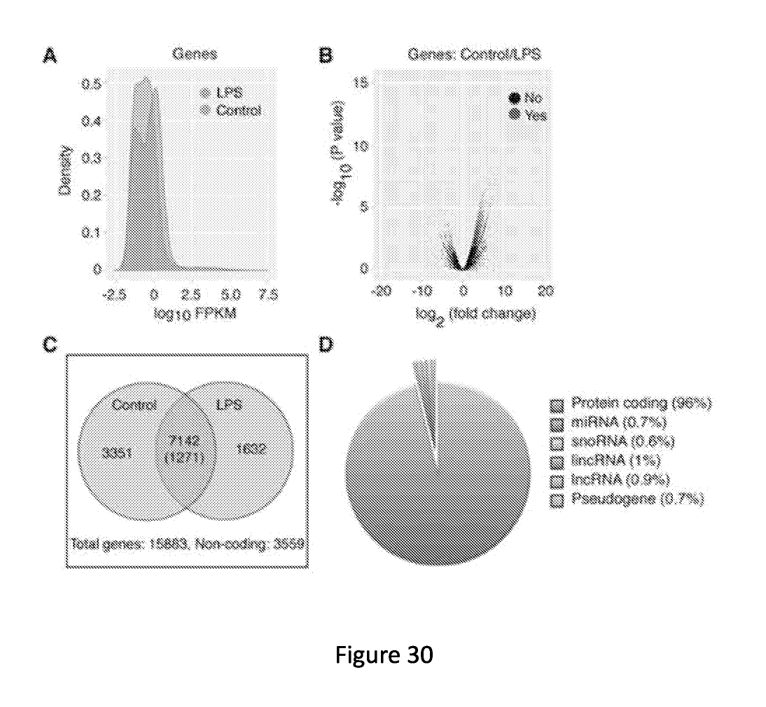

Quantitative PCR (qPCR) was used to validate 7 mRNAs including Gapdh; 4 mRNAs whose protein product was detected in exosomes (Cxcl2, Ccl2, Ccl4, and Tnfa, see below); and 2 mRNAs encoding transcription factors (Zeb2 and Creb). All transcripts were detected in both samples and Cxcl2 increased significantly after LPS stimulation as seen in the NGS data (FIG. 32).

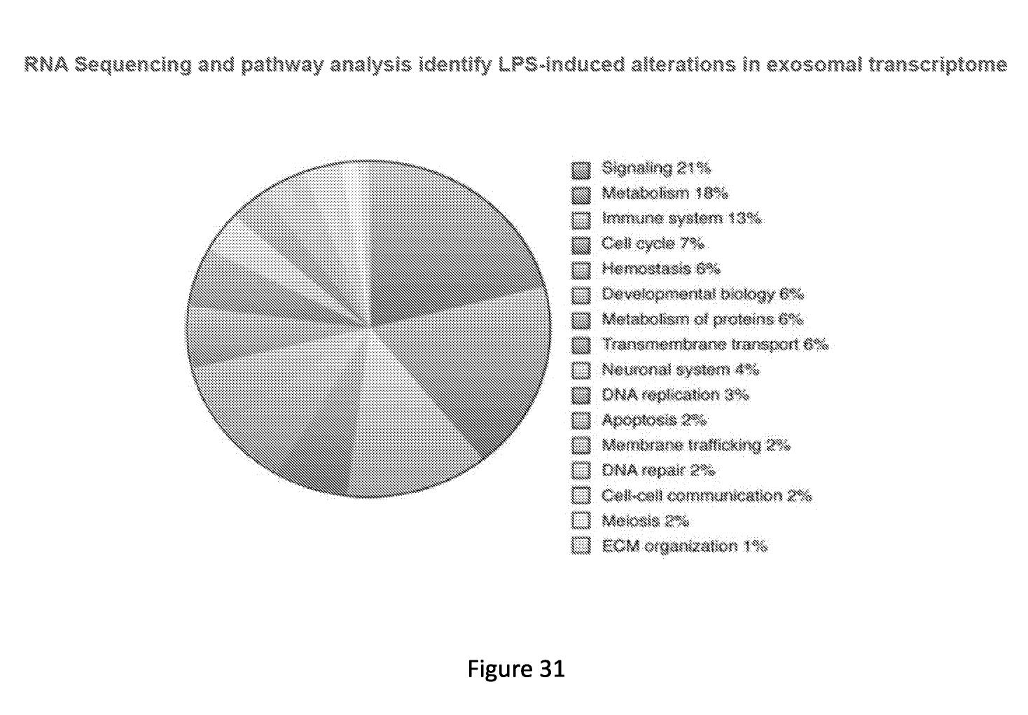

Experiments were conducted to assess miRNA target binding validation. The effect of miR-939 on the relative luciferase expression of four putative miR-939 targets (TNFAIP1, NOS2A, TNFα, and VEGFA) (FIG. 33A) and the effect of miR-532 on the relative luciferase expression on three miR-532 targets (CXCL3, PTGER2, PTGER3) were determined (FIG. 33B).

### LPS Stimulation Leads to Increased Exosomal Cytokines

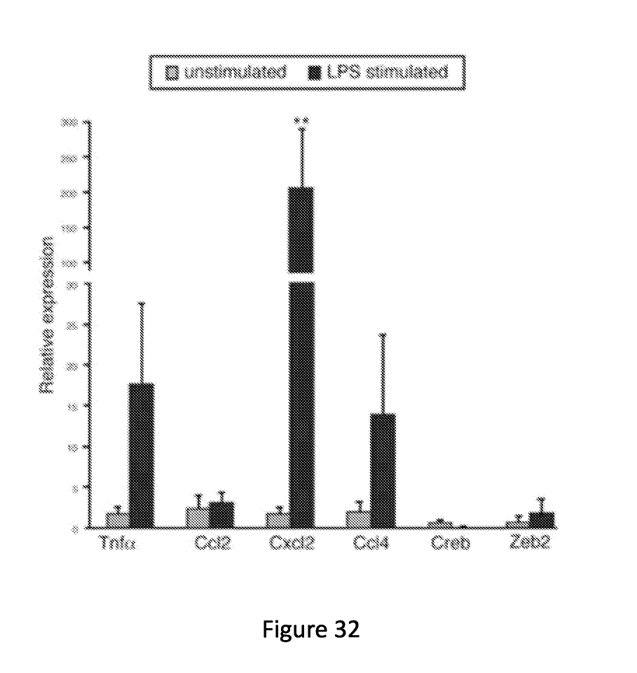

After LPS treatment, macrophages secrete a variety of chemokines and cytokines that induce the synthesis of additional pro- and anti-inflammatory mediators and act as homing signals for other immune cells. Without being bound to a particular theory, stimulation of macrophages with LPS leads to secretion of exosomes carrying a unique cytokine signature that could “prime” the recipient cell for an immune challenge. Consistent with previous studies, LPS stimulation of RAW 264.7 mouse macrophage cells led to the secretion of cytokines into culture media after 24 hr. Of the 16 cytokines secreted by RAW 264.7 cells after LPS stimulation, 10 were detected in RAW 264.7 cell-derived exosomes (FIGS. 34A-34C). Four proinflammatory cytokines were excluded from exosomes but present in the media (IL-1a, IL-1b, GM-CSF, and IL-6). Those present in LPS-treated exosomes include 2 anti-inflammatory mediators (G-CSF and IL-1Ra), as well as TNFa and a variety of chemokines. Both CCL3 and CCL4 were present in untreated exosomes as well as in LPS-treated exosomes.

### In Vitro Studies of Exosomal Function

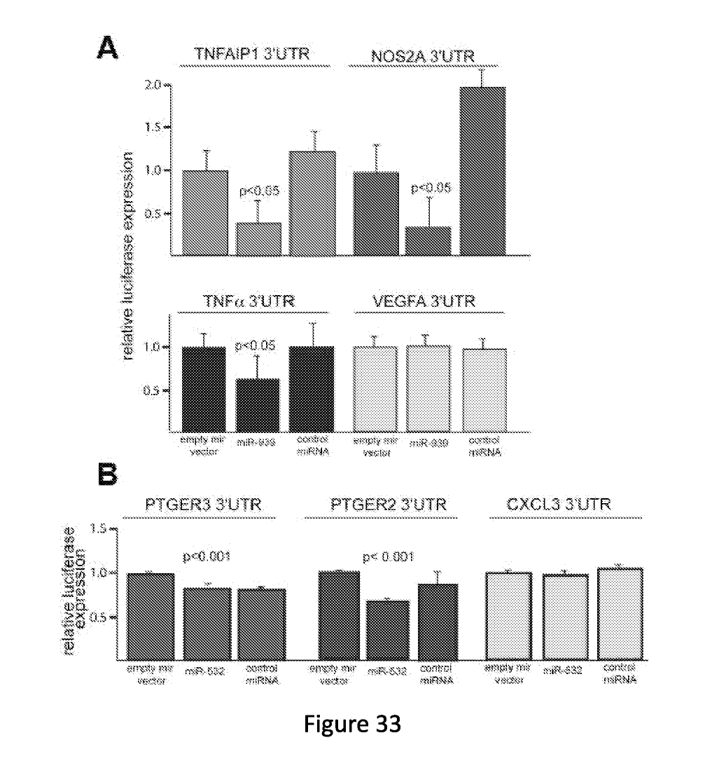

In addition to increased cytokines and LPS-responsive RNAs, exosomes from LPS-stimulated RAW 264.7 cells contained many miRNAs and mRNAs in common with those derived from naive cells. This led to the investigation of whether the LPS-induced signature transfers functionality to the recipient cell. Using RAW-Blue cells (InvivoGen; San Diego, Calif.), which have an inducible, chromosomally integrated secreted alkaline phosphatase (SEAP) gene downstream of the NF-κB promoter, the dose-dependent activation of NF-κB was studied by treatment with exosomes purified from culture media of naive or LPS-stimulated RAW 264.7 cells (FIG. 35). Using exosomal protein concentration to determine dose, 4 concentrations of exosomes from naive or LPS-stimulated cells were added to RAW-Blue cells. After a 24 hr incubation, the culture media was assayed. Exosomes from LPS-stimulated cells caused significantly more NF-κB activation at 50- and 100-μg/ml concentrations compared with exosomes from control cells. The results demonstrated that purified exosomes were functional and that the exosomes derived from LPS-stimulated macrophages induced dose-dependent activation of NF-κB.

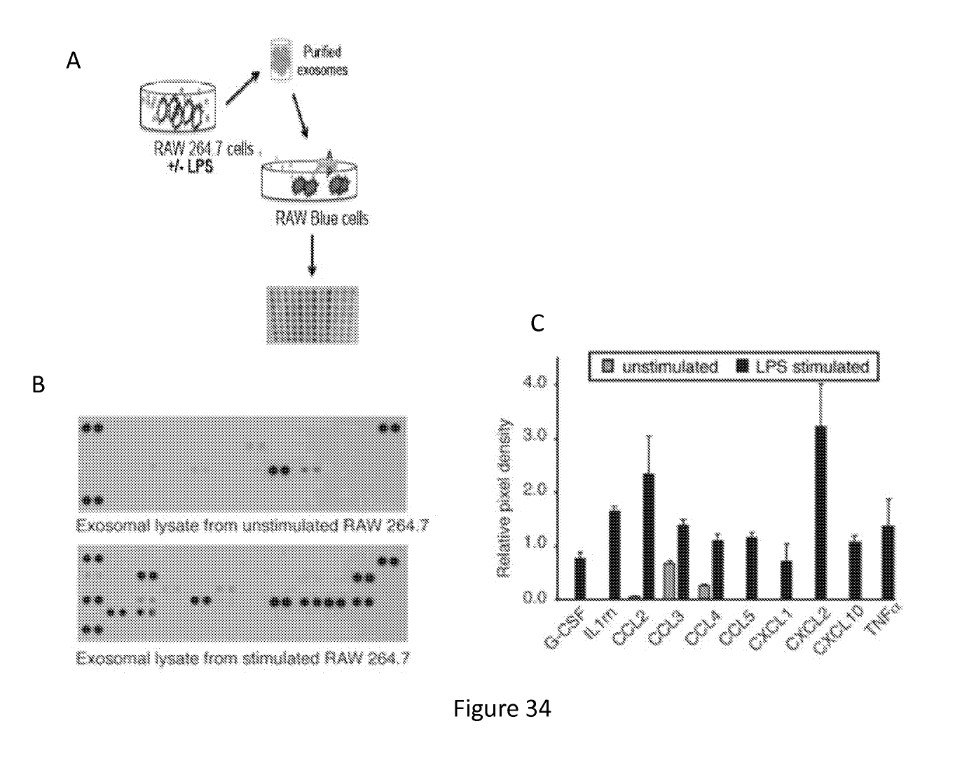

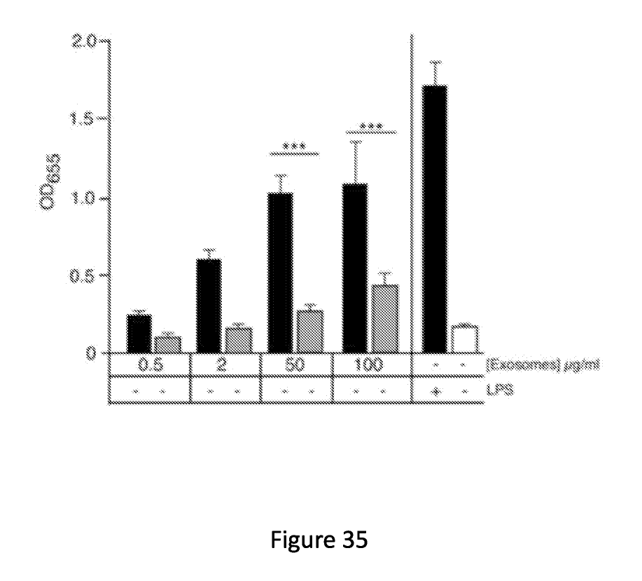

To determine the influence of macrophage-derived exosomes on inflammatory pain, a mouse model of inflammatory pain was used, involving inducing inflammatory pain by intraplantar injection of CFA suspension of heat-killed Mycobacterium tuberculosis into the hind paw (FIG. 36A). Baseline paw thickness was measured, thermal and mechanical sensitivities in 8-week-old male C57BL/6 mice were established, and then the inflammatory pain model was initiated. CFA administration led to paw swelling as well as mechanical and thermal hypersensitivity within 1 hr, while saline treatment did not result in hypersensitivity or swelling. Three hours after the administration of CFA or saline, mice were given another hind paw injection of 20 μl PBS or 0.5 μg of exosomes purified from either LPS-stimulated or untreated RAW 264.7 cells. This concentration was the maximum volume that could be administered to a mouse paw for the second injection while maintaining consistency of purified exosomes. No swelling was observed in saline-treated animals due to exosome injection (FIG. 36B). A single injection of exosomes from LPS-stimulated macrophages resulted in a significant reduction in paw thickness at 24 hr (FIG. 36C). Exosomes from untreated RAW 264.7 cells did not alter CFA-induced paw swelling.

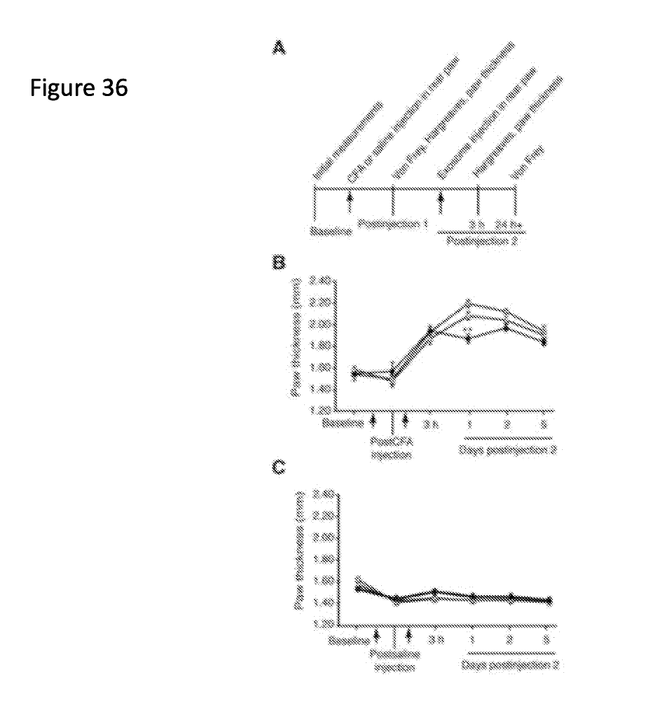

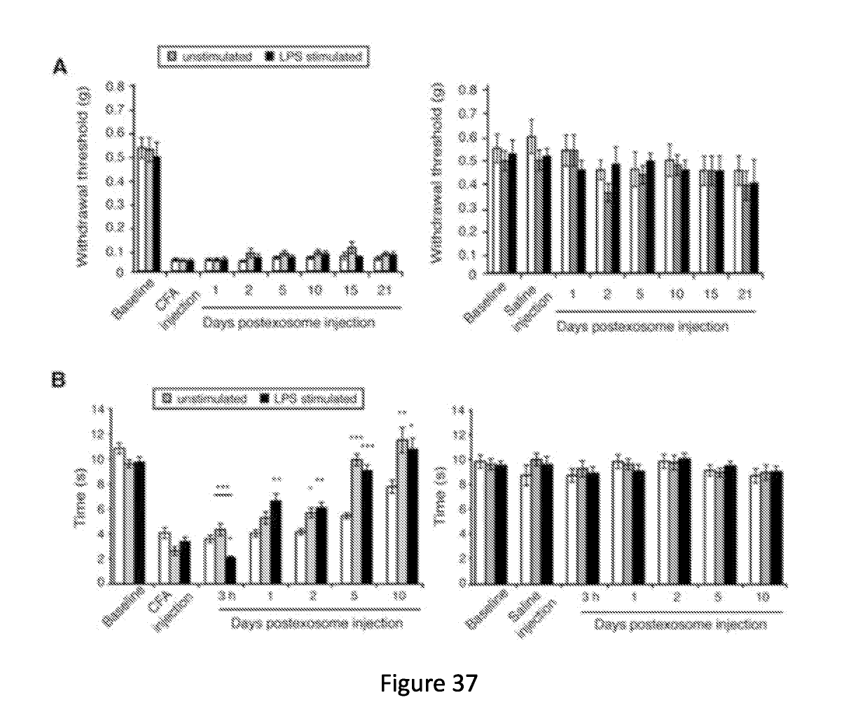

Measurement of mechanical sensitivity using von Frey filaments, beginning 24 hr after exosome injection, showed that exosomes did not have an effect on mechanical allodynia induced by CFA (FIG. 37A). Exosomes did not induce significant changes in paw withdrawal threshold in saline-treated animals, indicating that an intraplantar injection of exosomes does not evoke mechanical hypersensitivity. Thermal sensitivity to a radiant heat source was also assessed using the Hargreaves method. CFA-treated animals became hypersensitive to heat within 1 hr of CFA injection whereas saline-treated animals did not (FIG. 37B). Saline-treated animals that received injections of exosomes had no hypersensitivity to heat (FIG. 37B, right panel). In CFA-treated animals, injection of exosomes purified from LPS-stimulated macrophages (left panel) induced a transient decrease in paw withdrawal latency 3 hr after exosome administration, indicating an increase in thermal hypersensitivity that was not observed in saline-treated animals. An injection of exosomes from untreated RAW 264.7 cells into either CFA- or saline-treated animals showed no effect on thermal hyperalgesia at 3 hr. By 24 hr, CFA-treated animals that received injections of exosomes from LPS-stimulated RAW 264.7 cells had increased paw withdrawal latency compared with CFA-treated animals that received PBS. At 48 hr, CFA-treated animals displayed reduction in thermal hyperalgesia in response to exosome administration from both LPS-stimulated and naive macrophages. Thus the reduction in thermal hypersensitivity observed after 48 hr was independent of the inflammatory status of the macrophages from which these exosomes were derived. This attenuation of thermal hyperalgesia was specific to exosome injections and was not observed in CFA-treated animals that received an injection of PBS. One injection of exosomes was sufficient to specifically reduce CFA-induced thermal hypersensitivity within 24 hr, accelerate the return to normal sensitivity, and reduce paw inflammation. Without being bound to a particular theory, these results indicated a protective role for exosomes derived from macrophages delivered to an inflamed paw.

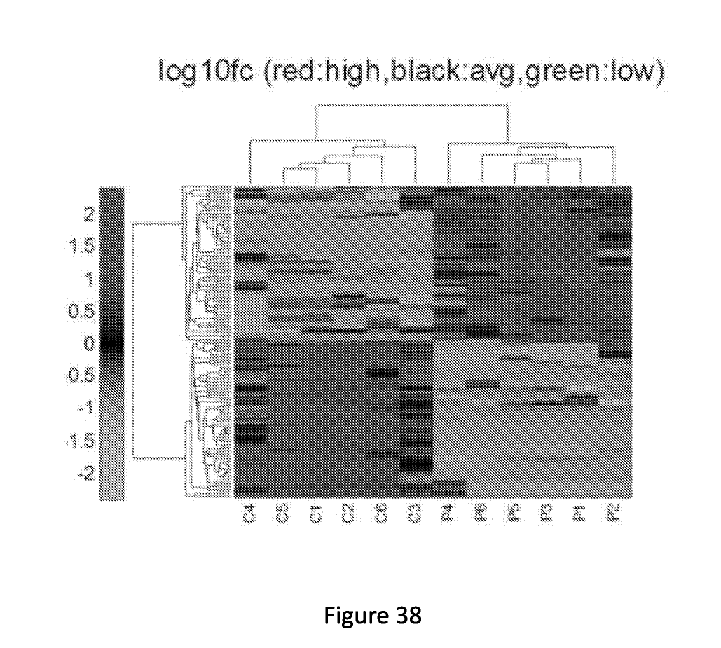

To determine whether exosomal miRNAs reflect the miRNA signature in whole blood of patients with CRPS, miRNAs in exosomes purified from the serum of 6 patients with CRPS and 6 healthy controls we analyzed (FIG. 38). Of the 12 subjects enrolled in this study, the patients with CRPS (5 women and 1 man) had a mean age of 37.8 years (range: 30-47 years), a mean disease duration of 7.7 years (range: 0.8-30 years), and reported median Numerical Rating Scale pain scores of 8.2 (range: 7-9). The 6 healthy control subjects (3 women and 3 men) had a mean age of 47.5 years (range: 32-69 years).

From a total of 503 miRNAs detected in at least one human serum-derived exosomal sample, 127 miRNAs were identified that were significantly different between CRPS and control-derived exosomes (Tables 9 and 10).

TABLE 9
Differentially expressed miRNAs in exosomes from patients with CRPS
compared with control subjects

| miRNA | p-value | fold change |
|---|---|---|
| hsa-miR-890 | 5.70E−08 | 1900.0 |
| hsa-miR-577 | 0.001 | 482.3 |
| hsa-miR-15b# | 4.00E−08 | 284.7 |
| hsa-miR-422a | 2.30E−07 | 204.3 |
| hsa-miR-1290 | 0.003 | 178.6 |
| hsa-miR-939 | 0.002 | 103.4 |
| hsa-miR-190 | 7.00E−08 | 77.9 |
| hsa-miR-32 | 1.30E−06 | 75.2 |
| hsa-miR-331-5p | 0.001 | 72.1 |
| hsa-miR-603 | 0.0003 | 71.7 |
| hsa-miR-129-5p | 2.70E−08 | 62.7 |
| hsa-miR-126# | 0.004 | 62.3 |
| hsa-miR-642 | 4.40E−07 | 57.5 |
| hsa-miR-342-5p | 2.70E−09 | 51.6 |
| hsa-miR-125a-3p | 8.90E−08 | 46.6 |
| hsa-miR-590-3P | 8.00E−05 | 40.0 |
| hsa-miR-301b | 9.70E−05 | 39.2 |
| hsa-miR-21# | 3.10E−06 | 38.2 |
| hsa-miR-409-5p | 1.60E−09 | 37.3 |
| hsa-miR-487b | 0.001 | 37.0 |
| hsa-miR-218 | 0.003 | 32.3 |
| hsa-miR-215 | 0.0001 | 31.0 |
| hsa-miR-210 | 2.50E−07 | 25.0 |
| hsa-miR-545 | 0.001 | 24.8 |
| hsa-miR-10b | 0.004 | 23.7 |
| hsa-miR-372 | 0.003 | 22.6 |
| hsa-miR-551b | 0.0004 | 21.6 |
| hsa-miR-339-5p | 0.0008 | 21.4 |
| hsa-miR-501-5p | 0.0004 | 21.0 |
| hsa-miR-382 | 0.003 | 20.9 |
| hsa-miR-1255B | 0.004 | 19.1 |
| hsa-miR-144 | 0.002 | 18.9 |
| hsa-miR-302b | 4.30E−06 | 17.4 |
| hsa-miR-31 | 0.002 | 16.2 |
| hsa-miR-601 | 0.0001 | 14.5 |
| hsa-miR-576-3p | 0.0004 | 14.1 |
| hsa-miR-1208 | 1.50E−05 | 12.8 |
| hsa-miR-320B | 0.002 | 12.6 |
| hsa-miR-541# | 0.0007 | 11.2 |
| hsa-miR-22# | 0.001 | 10.3 |
| hsa-miR-10b# | 0.003 | 10.1 |
| hsa-miR-15a | 0.0002 | 9.4 |
| hsa-miR-660 | 0.007 | 8.2 |
| hsa-miR-223# | 0.006 | 8.0 |
| hsa-miR-93# | 0.008 | 7.5 |
| hsa-miR-130b | 0.0003 | 7.5 |
| hsa-miR-625 | 0.004 | 6.8 |
| hsa-miR-330-3p | 0.0003 | 6.8 |
| hsa-miR-212 | 0.008 | 6.6 |
| hsa-miR-9 | 0.0003 | 6.4 |
| hsa-miR-520c-3p | 0.002 | 6.4 |
| hsa-miR-378 | 0.007 | 5.9 |
| hsa-miR-886-5p | 0.002 | 5.7 |
| hsa-miR-200c | 0.006 | 4.7 |
| hsa-miR-361-5p | 0.001 | 4.7 |
| hsa-miR-98 | 0.003 | 4.4 |
| hsa-miR-509-5p | 0.001 | 4.2 |
| hsa-miR-654-5p | 0.0002 | 3.4 |
| hsa-miR-140-3p | 0.009 | 3.3 |
| hsa-miR-100 | 0.007 | 2.9 |
| hsa-miR-502-5p | 0.0008 | 2.7 |
| hsa-miR-874 | 0.0006 | 2.4 |
| hsa-miR-497 | −0.005 | −1.7 |
| hsa-miR-588 | −0.005 | −2.0 |
| hsa-miR-770-5p | −0.002 | −2.7 |
| hsa-miR-99b# | −0.0008 | −2.9 |
| hsa-miR-1182 | −0.0002 | −3.3 |
| hsa-miR-1303 | −0.0004 | −3.5 |
| hsa-miR-204 | −0.003 | −3.5 |
| hsa-miR-1269 | −0.0003 | −3.6 |
| hsa-miR-628-3p | −0.003 | −3.7 |
| hsa-miR-25# | −0.0006 | −3.8 |
| hsa-miR-30d# | −0.0006 | −4.1 |
| hsa-miR-29c | −0.007 | −6.8 |
| hsa-miR-646 | −0.0001 | −6.9 |
| hsa-miR-26b# | −0.004 | −7.6 |
| hsa-miR-491-5p | −0.002 | −7.9 |
| hsa-miR-758 | −0.0003 | −9.1 |
| hsa-miR-513C | −2.50E−06 | −10.2 |
| hsa-miR-1253 | −0.004 | −14.3 |
| hsa-miR-636 | −0.0005 | −15.3 |
| hsa-miR-1282 | −1.00E−06 | −15.4 |
| RNU48 | −0.002 | −15.8 |
| hsa-miR-433 | −0.009 | −11.9 |
| hsa-miR-485-3p | −4.80E−05 | −12.3 |
| hsa-miR-200b | −0.002 | −12.7 |
| hsa-miR-455-3p | −1.20E−06 | −16.2 |
| hsa-miR-1227 | −6.90E−06 | −21.6 |
| hsa-miR-184 | −1.00E−07 | −22.3 |
| hsa-miR-489 | −0.0001 | −22.9 |
| hsa-miR-512-3p | −1.60E−07 | −27.8 |
| hsa-miR-34a | −2.50E−06 | −30.4 |
| hsa-miR-367 | −0.003 | −31.7 |
| hsa-miR-645 | −0.007 | −33.2 |
| hsa-miR-767-5p | −4.20E−06 | −34.8 |
| hsa-miR-506 | −0.0004 | −37.6 |
| hsa-miR-522 | −0.0009 | −38.1 |
| hsa-miR-20a# | −2.70E−05 | −39.2 |
| hsa-miR-23a# | −5.30E−05 | −40.6 |
| hsa-miR-744# | −0.0003 | −40.7 |
| hsa-miR-630 | −8.20E−05 | −42.4 |
| hsa-miR-518e | −1.80E−05 | −42.4 |
| hsa-miR-520b | −1.40E−05 | −43.7 |
| hsa-miR-511 | −0.0008 | −44.6 |
| hsa-miR-337-5p | −3.30E−05 | −50.4 |
| hsa-miR-190b | −0.0006 | −58.1 |
| hsa-miR-650 | −0.007 | −63.6 |
| hsa-miR-639 | −0.002 | −64.1 |
| hsa-miR-379 | −6.40E−11 | −76.3 |
| hsa-miR-302c | −0.005 | −101.8 |
| hsa-miR-34a# | −6.50E−07 | −110.0 |
| hsa-miR-656 | −0.003 | −110.7 |
| hsa-miR-1259 | −0.0004 | −141.5 |
| hsa-miR-518f | −7.40E−05 | −145.3 |
| hsa-miR-432 | −0.0001 | −155.5 |
| hsa-miR-572 | −2.50E−05 | −161.9 |
| hsa-miR-615-3p | −2.80E−07 | −199.5 |
| hsa-miR-1294 | −7.00E−06 | −201.9 |
| hsa-miR-154# | −3.80E−06 | −3341.1 |
| hsa-miR-1276 | −1.70E−07 | −268.6 |
| hsa-miR-519a | −0.004 | −279.6 |
| hsa-miR-324-3p | −4.70E−05 | −311.1 |
| hsa-miR-872 | −3.00E−05 | −373.8 |
| hsa-miR-1233 | −4.60E−05 | −451.9 |
| hsa-miR-302d | −3.20E−05 | −1332.2 |
| hsa-miR-1276 | −1.70E−07 | −268.6 |
| hsa-miR-519a | −0.004 | −279.6 |
| hsa-miR-324-3p | −4.70E−05 | −311.1 |
| hsa-miR-872 | −3.00E−05 | −373.8 |
| hsa-miR-1233 | −4.60E−05 | −451.9 |
| hsa-miR-302d | −3.20E−05 | −1332.2 |
| hsa-miR-154# | −3.80E−06 | −3341.1 |

miRNAs shown in bold were identified in our previous study to be differentially expressed in whole blood from patients with CRPS.
Significance was determined by applying the Benjamini-Hochberg false discovery rate correction to the results of a2-tailed t test.

TABLE 10
All miRNAs detected in exosomes from human serum for control subjects
and patients with CRPS.

| | # of | # of | | | |
|---|---|---|---|---|---|
| miRNA | controls | patients | ddct | p-value | fold-change |
| hsa-let-7a | 4 | 5 | −0.728 | 0.489 | 1.657 |
| hsa-let-7c | 3 | 3 | −0.365 | 0.557 | 1.288 |
| RNU44 | 1 | 0 | n/a | n/a | n/a |
| hsa-miR-126 | 5 | 6 | −0.053 | 0.952 | 1.037 |
| RNU48 | 4 | 3 | 2.147 | 0.032 | −4.429 |
| hsa-miR-320 | 5 | 6 | 0.953 | 0.420 | −1.936 |
| hsa-let-7b | 5 | 6 | −0.610 | 0.806 | 1.527 |
| hsa-miR-532-3p | 4 | 4 | −0.332 | 0.566 | 1.259 |
| hsa-miR-939 | 2 | 5 | −6.692 | 0.002 | 103.420 |
| hsa-miR-425# | 1 | 1 | −0.664 | 0.670 | 1.585 |
| hsa-miR-181a-2# | 1 | 0 | n/a | n/a | n/a |
| hsa-miR-625# | 5 | 5 | −1.166 | 0.295 | 2.243 |
| hsa-miR-25# | 2 | 0 | n/a | n/a | n/a |
| hsa-miR-320B | 2 | 2 | −3.650 | 0.002 | 12.556 |
| hsa-miR-720 | 5 | 5 | 1.145 | 0.585 | −2.212 |
| hsa-miR-629 | 1 | 1 | −0.764 | 0.559 | 1.698 |
| hsa-miR-664 | 3 | 4 | −3.472 | 0.029 | 11.094 |
| hsa-let-7d | 4 | 6 | 1.586 | 0.458 | −3.003 |
| hsa-let-7e | 4 | 6 | 1.205 | 0.305 | −2.306 |
| hsa-let-7f | 2 | 4 | 0.385 | 0.846 | −1.306 |
| hsa-let-7g | 4 | 5 | 0.985 | 0.421 | −1.979 |
| hsa-miR-1 | 2 | 3 | −1.311 | 0.472 | 2.482 |
| hsa-miR-9 | 2 | 4 | −2.671 | 0.000 | 6.369 |
| hsa-miR-10a | 4 | 4 | 0.325 | 0.825 | −1.252 |
| hsa-miR-10b | 1 | 3 | −4.566 | 0.004 | 23.685 |
| MammU6 | 6 | 6 | −0.780 | 0.698 | 1.718 |
| hsa-miR-15a | 2 | 4 | −3.238 | 0.000 | 9.438 |
| hsa-miR-15b | 4 | 5 | −2.574 | 0.039 | 5.954 |
| hsa-miR-16 | 5 | 6 | −0.067 | 0.949 | 1.047 |
| hsa-miR-17 | 5 | 6 | −0.692 | 0.365 | 1.616 |
| hsa-miR-18a | 4 | 5 | −1.415 | 0.125 | 2.667 |
| hsa-miR-18b | 2 | 5 | −1.045 | 0.362 | 2.063 |
| hsa-miR-19a | 5 | 6 | −0.972 | 0.477 | 1.962 |
| hsa-miR-19b | 5 | 6 | −2.236 | 0.147 | 4.712 |
| hsa-miR-20a | 5 | 6 | 0.502 | 0.791 | −1.416 |
| hsa-miR-20b | 4 | 5 | −0.279 | 0.777 | 1.213 |
| hsa-miR-21 | 4 | 6 | 0.465 | 0.580 | −1.380 |
| hsa-miR-22 | 1 | 4 | −2.354 | 0.237 | 5.114 |
| hsa-miR-23a | 2 | 2 | 0.995 | 0.293 | −1.993 |
| hsa-miR-23b | 1 | 3 | −2.542 | 0.031 | 5.826 |
| hsa-miR-24 | 5 | 6 | −1.008 | 0.430 | 2.011 |
| hsa-miR-25 | 4 | 5 | 0.611 | 0.549 | −1.527 |
| hsa-miR-26a | 4 | 5 | 1.827 | 0.149 | −3.548 |
| hsa-miR-26b | 4 | 6 | 3.280 | 0.044 | −9.712 |
| hsa-miR-27a | 4 | 5 | −1.868 | 0.071 | 3.651 |
| hsa-miR-27b | 3 | 5 | 0.879 | 0.095 | −1.839 |
| hsa-miR-28-3p | 4 | 5 | −1.015 | 0.405 | 2.021 |
| hsa-miR-28-5p | 3 | 3 | −0.996 | 0.252 | 1.994 |
| hsa-miR-29a | 4 | 6 | 2.897 | 0.086 | −7.451 |
| hsa-miR-29b | 1 | 0 | n/a | n/a | n/a |
| hsa-miR-29c | 3 | 3 | 2.773 | 0.007 | −6.834 |
| hsa-miR-30b | 5 | 6 | −0.241 | 0.854 | 1.182 |
| hsa-miR-30c | 5 | 6 | −0.080 | 0.947 | 1.057 |
| hsa-miR-31 | 1 | 2 | −4.015 | 0.002 | 16.173 |
| hsa-miR-32 | 0 | 2 | n/a | n/a | n/a |
| hsa-miR-33b | 0 | 1 | n/a | n/a | n/a |
| hsa-miR-34a | 2 | 1 | 4.928 | 0.000 | −30.442 |
| hsa-miR-92a | 5 | 5 | −2.079 | 0.076 | 4.226 |
| hsa-miR-93 | 4 | 6 | 0.408 | 0.758 | −1.327 |
| hsa-miR-95 | 4 | 2 | −0.797 | 0.067 | 1.737 |
| hsa-miR-96 | 1 | 0 | n/a | n/a | n/a |
| hsa-miR-98 | 1 | 4 | −2.145 | 0.003 | 4.422 |
| hsa-miR-99a | 1 | 2 | −1.113 | 0.458 | 2.162 |
| hsa-miR-99b | 3 | 5 | 0.965 | 0.145 | −1.951 |
| hsa-miR-100 | 2 | 2 | −1.543 | 0.007 | 2.914 |
| hsa-miR-101 | 2 | 4 | −0.276 | 0.673 | 1.211 |
| hsa-miR-103 | 4 | 5 | −0.631 | 0.354 | 1.548 |
| hsa-miR-106a | 5 | 6 | −1.166 | 0.244 | 2.243 |
| hsa-miR-106b | 4 | 6 | 1.525 | 0.116 | −2.878 |
| hsa-miR-107 | 2 | 5 | −0.212 | 0.840 | 1.158 |
| hsa-miR-122 | 4 | 5 | −0.504 | 0.558 | 1.419 |
| hsa-miR-124 | 0 | 1 | n/a | n/a | n/a |
| hsa-miR-125a-3p | 0 | 2 | n/a | n/a | n/a |
| hsa-miR-125a-5p | 3 | 4 | 2.418 | 0.124 | −5.345 |
| hsa-miR-125b | 3 | 4 | 0.374 | 0.588 | −1.296 |
| hsa-miR-127-3p | 3 | 5 | 1.276 | 0.238 | −2.423 |
| hsa-miR-128 | 2 | 4 | −0.422 | 0.526 | 1.340 |
| hsa-miR-129-5p | 0 | 2 | n/a | n/a | n/a |
| hsa-miR-130a | 4 | 5 | 0.351 | 0.847 | −1.275 |
| hsa-miR-130b | 3 | 5 | −2.897 | 0.000 | 7.450 |
| hsa-miR-132 | 3 | 5 | −0.306 | 0.845 | 1.236 |
| hsa-miR-133a | 3 | 3 | −0.832 | 0.410 | 1.780 |
| hsa-miR-133b | 1 | 1 | 0.375 | 0.766 | −1.297 |
| hsa-miR-134 | 4 | 5 | −1.317 | 0.241 | 2.491 |
| hsa-miR-135a | 1 | 2 | −1.464 | 0.291 | 2.759 |
| hsa-miR-135b | 1 | 0 | n/a | n/a | n/a |
| hsa-miR-139-3p | 2 | 3 | 2.429 | 0.020 | −5.386 |
| hsa-miR-139-5p | 5 | 6 | 0.964 | 0.445 | −1.951 |
| hsa-miR-140-3p | 5 | 3 | −1.720 | 0.009 | 3.295 |
| hsa-miR-140-5p | 5 | 6 | −1.332 | 0.182 | 2.518 |
| hsa-miR-141 | 0 | 1 | n/a | n/a | n/a |
| hsa-miR-142-3p | 5 | 6 | −0.805 | 0.574 | 1.747 |
| hsa-miR-142-5p | 3 | 5 | 0.945 | 0.131 | −1.925 |
| hsa-miR-143 | 4 | 3 | −0.743 | 0.340 | 1.673 |
| hsa-miR-145 | 3 | 5 | −1.411 | 0.309 | 2.659 |
| hsa-miR-146a | 5 | 6 | −0.480 | 0.671 | 1.395 |
| hsa-miR-146b-3p | 1 | 0 | n/a | n/a | n/a |
| hsa-miR-146b-5p | 5 | 6 | −0.096 | 0.934 | 1.069 |
| hsa-miR-147b | 1 | 0 | n/a | n/a | n/a |
| hsa-miR-148a | 2 | 3 | −0.811 | 0.290 | 1.755 |
| hsa-miR-148b | 2 | 4 | 0.976 | 0.376 | −1.966 |
| hsa-miR-149 | 1 | 2 | 0.331 | 0.764 | −1.258 |
| hsa-miR-150 | 5 | 6 | 0.193 | 0.787 | −1.143 |
| hsa-miR-152 | 4 | 3 | −1.364 | 0.097 | 2.574 |
| hsa-miR-181a | 3 | 5 | 1.596 | 0.099 | −3.023 |
| hsa-miR-181c | 1 | 0 | n/a | n/a | n/a |
| hsa-miR-182 | 3 | 3 | 0.232 | 0.792 | −1.175 |
| hsa-miR-183 | 2 | 4 | −1.532 | 0.010 | 2.893 |
| hsa-miR-184 | 2 | 0 | n/a | n/a | n/a |
| hsa-miR-185 | 5 | 5 | −1.120 | 0.117 | 2.173 |
| hsa-miR-186 | 5 | 6 | 1.099 | 0.588 | −2.142 |
| hsa-miR-187 | 0 | 1 | n/a | n/a | n/a |
| hsa-miR-188-3p | 0 | 1 | n/a | n/a | n/a |
| hsa-miR-190 | 1 | 2 | −6.284 | 0.000 | 77.920 |
| hsa-miR-191 | 5 | 6 | 0.205 | 0.614 | −1.153 |
| hsa-miR-192 | 4 | 5 | 0.145 | 0.897 | −1.106 |
| hsa-miR-193a-3p | 1 | 1 | −0.418 | 0.722 | 1.336 |
| hsa-miR-193a-5p | 2 | 4 | 1.481 | 0.123 | −2.792 |
| hsa-miR-193b | 3 | 5 | 1.774 | 0.220 | −3.421 |
| hsa-miR-194 | 4 | 5 | 0.267 | 0.704 | −1.203 |
| hsa-miR-195 | 4 | 5 | 2.466 | 0.029 | −5.526 |
| hsa-miR-196b | 3 | 4 | −1.887 | 0.290 | 3.700 |
| hsa-miR-197 | 4 | 5 | 2.783 | 0.192 | −6.881 |
| hsa-miR-199a-5p | 1 | 1 | 1.032 | 0.366 | −2.045 |
| hsa-miR-199a-3p | 4 | 5 | −0.499 | 0.604 | 1.413 |
| hsa-miR-199b-5p | 1 | 1 | 0.650 | 0.431 | −1.569 |
| hsa-miR-200a | 0 | 1 | n/a | n/a | n/a |
| hsa-miR-200b | 2 | 1 | 3.670 | 0.002 | −12.732 |
| hsa-miR-200c | 3 | 3 | −2.232 | 0.006 | 4.698 |
| hsa-miR-202 | 0 | 1 | n/a | n/a | n/a |
| hsa-miR-204 | 3 | 3 | 1.823 | 0.003 | −3.538 |
| hsa-miR-205 | 0 | 1 | n/a | n/a | n/a |
| hsa-miR-210 | 0 | 3 | n/a | n/a | n/a |
| hsa-miR-214 | 3 | 2 | −0.180 | 0.621 | 1.133 |
| hsa-miR-215 | 0 | 2 | n/a | n/a | n/a |
| hsa-miR-218 | 1 | 3 | −5.015 | 0.003 | 32.331 |
| hsa-miR-219-5p | 1 | 1 | 0.176 | 0.382 | −1.130 |
| hsa-miR-221 | 4 | 5 | −2.836 | 0.021 | 7.140 |
| hsa-miR-222 | 6 | 6 | −2.118 | 0.191 | 4.341 |
| hsa-miR-223 | 5 | 6 | −1.533 | 0.074 | 2.894 |
| hsa-miR-224 | 3 | 2 | −1.035 | 0.144 | 2.049 |
| hsa-miR-296-5p | 1 | 1 | 0.124 | 0.801 | −1.089 |
| hsa-miR-299-3p | 1 | 0 | n/a | n/a | n/a |
| hsa-miR-299-5p | 0 | 1 | n/a | n/a | n/a |
| hsa-miR-301a | 3 | 5 | 2.096 | 0.147 | −4.276 |
| hsa-miR-301b | 1 | 3 | −5.292 | 0.000 | 39.175 |
| hsa-miR-302a | 2 | 1 | 1.703 | 0.084 | −3.256 |
| hsa-miR-302b | 0 | 2 | n/a | n/a | n/a |
| hsa-miR-302c | 3 | 3 | 6.670 | 0.005 | −101.848 |
| hsa-miR-323-3p | 4 | 5 | −1.176 | 0.155 | 2.259 |
| hsa-miR-324-3p | 3 | 5 | 8.281 | 0.000 | −311.120 |
| hsa-miR-324-5p | 2 | 5 | 1.666 | 0.113 | −3.174 |
| hsa-miR-328 | 3 | 4 | 2.281 | 0.076 | −4.859 |
| hsa-miR-329 | 1 | 0 | n/a | n/a | n/a |
| hsa-miR-330-3p | 1 | 2 | −2.756 | 0.000 | 6.755 |
| hsa-miR-331-3p | 5 | 5 | −3.135 | 0.017 | 8.786 |
| hsa-miR-331-5p | 1 | 3 | −6.172 | 0.001 | 72.090 |
| hsa-miR-335 | 3 | 5 | −0.026 | 0.970 | 1.018 |
| hsa-miR-337-5p | 2 | 2 | 5.654 | 0.000 | −50.346 |
| hsa-miR-338-3p | 0 | 1 | n/a | n/a | n/a |
| hsa-miR-339-3p | 3 | 5 | −0.076 | 0.913 | 1.054 |
| hsa-miR-339-5p | 1 | 3 | −4.423 | 0.001 | 21.444 |
| hsa-miR-340 | 3 | 3 | −1.678 | 0.124 | 3.201 |
| has-miR-155 | 4 | 6 | 0.669 | 0.430 | −1.590 |
| hsa-miR-342-3p | 5 | 6 | −0.666 | 0.654 | 1.586 |
| hsa-miR-342-5p | 0 | 2 | n/a | n/a | n/a |
| hsa-miR-345 | 3 | 4 | −1.288 | 0.074 | 2.441 |
| hsa-miR-361-5p | 2 | 2 | −2.223 | 0.001 | 4.668 |
| hsa-miR-362-5p | 3 | 4 | 1.217 | 0.255 | −2.325 |
| hsa-miR-363 | 3 | 2 | 0.262 | 0.609 | −1.199 |
| hsa-miR-365 | 4 | 4 | −0.786 | 0.216 | 1.724 |
| hsa-miR-367 | 2 | 1 | 4.988 | 0.003 | −31.736 |
| hsa-miR-369-3p | 1 | 0 | n/a | n/a | n/a |
| hsa-miR-370 | 2 | 4 | 0.901 | 0.344 | −1.867 |
| hsa-miR-372 | 0 | 3 | n/a | n/a | n/a |
| hsa-miR-373 | 0 | 1 | n/a | n/a | n/a |
| hsa-miR-374a | 5 | 6 | 3.666 | 0.119 | −12.692 |
| hsa-miR-374b | 5 | 6 | 1.358 | 0.370 | −2.563 |
| hsa-miR-375 | 1 | 2 | −2.515 | 0.120 | 5.718 |
| hsa-miR-376a | 4 | 3 | 0.027 | 0.985 | −1.019 |
| hsa-miR-376b | 1 | 1 | 0.028 | 0.951 | −1.019 |
| hsa-miR-379 | 2 | 0 | n/a | n/a | n/a |
| hsa-miR-380 | 1 | 0 | n/a | n/a | n/a |
| hsa-miR-381 | 2 | 1 | 2.429 | 0.112 | −5.384 |
| hsa-miR-382 | 2 | 5 | −4.384 | 0.003 | 20.873 |
| hsa-miR-409-5p | 0 | 2 | n/a | n/a | n/a |
| hsa-miR-410 | 2 | 3 | −0.363 | 0.439 | 1.286 |
| hsa-miR-411 | 2 | 4 | 3.388 | 0.002 | −10.466 |
| hsa-miR-422a | 0 | 2 | n/a | n/a | n/a |
| hsa-miR-423-5p | 3 | 5 | 2.548 | 0.022 | −5.849 |
| hsa-miR-425 | 5 | 5 | −0.896 | 0.420 | 1.861 |
| hsa-miR-429 | 1 | 1 | 0.504 | 0.568 | −1.418 |
| hsa-miR-431 | 0 | 3 | n/a | n/a | n/a |
| hsa-miR-433 | 3 | 1 | 3.578 | 0.009 | −11.942 |
| hsa-miR-449a | 1 | 0 | n/a | n/a | n/a |
| hsa-miR-450a | 0 | 1 | n/a | n/a | n/a |
| hsa-miR-450b-3p | 2 | 3 | 2.790 | 0.024 | −6.918 |
| hsa-miR-450b-5p | 3 | 3 | 1.620 | 0.189 | −3.073 |
| hsa-miR-451 | 5 | 6 | −1.767 | 0.252 | 3.404 |
| hsa-miR-452 | 1 | 2 | −3.221 | 0.015 | 9.322 |
| hsa-miR-454 | 5 | 6 | 0.450 | 0.810 | −1.366 |
| hsa-miR-455-3p | 2 | 0 | n/a | n/a | n/a |
| hsa-miR-483-5p | 4 | 5 | 4.152 | 0.028 | −17.782 |
| hsa-miR-484 | 5 | 6 | −0.550 | 0.378 | 1.464 |
| hsa-miR-485-3p | 3 | 3 | 3.623 | 0.000 | −12.323 |
| hsa-miR-485-5p | 0 | 1 | n/a | n/a | n/a |
| hsa-miR-486-3p | 3 | 5 | −2.507 | 0.015 | 5.686 |
| hsa-miR-486-5p | 5 | 6 | 0.893 | 0.541 | −1.857 |
| hsa-miR-487a | 1 | 0 | n/a | n/a | n/a |
| hsa-miR-487b | 1 | 3 | −5.209 | 0.001 | 36.983 |
| hsa-miR-488 | 1 | 0 | n/a | n/a | n/a |
| hsa-miR-489 | 2 | 0 | n/a | n/a | n/a |
| hsa-miR-490-3p | 1 | 0 | n/a | n/a | n/a |
| hsa-miR-491-5p | 2 | 3 | 2.978 | 0.002 | −7.878 |
| hsa-miR-493 | 1 | 1 | −0.665 | 0.483 | 1.586 |
| hsa-miR-494 | 2 | 4 | 1.441 | 0.041 | −2.715 |
| hsa-miR-495 | 2 | 4 | 2.060 | 0.030 | −4.169 |
| hsa-miR-499-3p | 1 | 1 | −0.453 | 0.785 | 1.369 |
| hsa-miR-499-5p | 0 | 1 | n/a | n/a | n/a |
| hsa-miR-500 | 3 | 4 | −1.940 | 0.321 | 3.838 |
| hsa-miR-501-5p | 0 | 3 | n/a | n/a | n/a |
| hsa-miR-502-3p | 0 | 1 | n/a | n/a | n/a |
| hsa-miR-502-5p | 1 | 2 | −1.405 | 0.001 | 2.648 |
| hsa-miR-507 | 1 | 0 | n/a | n/a | n/a |
| hsa-miR-508-3p | 0 | 1 | n/a | n/a | n/a |
| hsa-miR-509-5p | 0 | 2 | n/a | n/a | n/a |
| hsa-miR-512-3p | 2 | 0 | n/a | n/a | n/a |
| hsa-miR-515-3p | 0 | 1 | n/a | n/a | n/a |
| hsa-miR-517a | 1 | 3 | −2.757 | 0.019 | 6.762 |
| hsa-miR-517c | 1 | 0 | n/a | n/a | n/a |
| hsa-miR-518b | 2 | 2 | 1.829 | 0.041 | −3.553 |
| hsa-miR-518d-3p | 4 | 6 | 1.670 | 0.363 | −3.182 |
| hsa-miR-518d-5p | 1 | 1 | n/a | n/a | n/a |
| hsa-miR-518e | 3 | 0 | n/a | n/a | n/a |
| hsa-miR-518f | 2 | 1 | 7.183 | 0.000 | −145.306 |
| hsa-miR-519a | 3 | 4 | 8.127 | 0.004 | −279.556 |
| hsa-miR-519d | 1 | 0 | n/a | n/a | n/a |
| hsa-miR-519e | 1 | 0 | n/a | n/a | n/a |
| hsa-miR-520a-5p | 1 | 1 | 0.702 | 0.431 | −1.626 |
| hsa-miR-520d-5p | 0 | 1 | n/a | n/a | n/a |
| hsa-miR-520g | 1 | 0 | n/a | n/a | n/a |
| hsa-miR-521 | 1 | 1 | −1.194 | 0.374 | 2.288 |
| hsa-miR-522 | 3 | 0 | n/a | n/a | n/a |
| hsa-miR-523 | 3 | 4 | 0.138 | 0.936 | −1.100 |
| hsa-miR-525-3p | 0 | 1 | n/a | n/a | n/a |
| hsa-miR-532-5p | 3 | 5 | −0.719 | 0.257 | 1.646 |
| hsa-miR-539 | 3 | 3 | −1.768 | 0.157 | 3.407 |
| hsa-miR-545 | 1 | 2 | −4.632 | 0.001 | 24.790 |
| hsa-miR-548a-3p | 1 | 1 | −0.856 | 0.528 | 1.810 |
| hsa-miR-548a-5p | 1 | 0 | n/a | n/a | n/a |
| hsa-miR-548b-5p | 1 | 0 | n/a | n/a | n/a |
| hsa-miR-548c-3p | 1 | 1 | −0.008 | 0.996 | 1.005 |
| hsa-miR-551b | 0 | 3 | n/a | n/a | n/a |
| hsa-miR-556-5p | 0 | 1 | n/a | n/a | n/a |
| hsa-miR-570 | 0 | 1 | n/a | n/a | n/a |
| hsa-miR-574-3p | 4 | 6 | 1.680 | 0.287 | −3.204 |
| hsa-miR-576-3p | 2 | 2 | −3.817 | 0.000 | 14.091 |
| hsa-miR-576-5p | 0 | 1 | n/a | n/a | n/a |
| hsa-miR-579 | 1 | 0 | n/a | n/a | n/a |
| hsa-miR-582-3p | 1 | 0 | n/a | n/a | n/a |
| hsa-miR-582-5p | 1 | 1 | 0.208 | 0.887 | −1.155 |
| hsa-miR-589 | 1 | 1 | −0.313 | 0.813 | 1.242 |
| hsa-miR-590-5p | 3 | 3 | −2.255 | 0.028 | 4.773 |
| hsa-miR-597 | 1 | 2 | −2.219 | 0.041 | 4.654 |
| hsa-miR-598 | 2 | 2 | 0.466 | 0.492 | −1.382 |
| hsa-miR-615-3p | 2 | 0 | n/a | n/a | n/a |
| hsa-miR-615-5p | 1 | 1 | −0.468 | 0.792 | 1.383 |
| hsa-miR-616 | 1 | 1 | n/a | n/a | n/a |
| hsa-miR-618 | 3 | 3 | 0.670 | 0.623 | −1.591 |
| hsa-miR-625 | 1 | 3 | −2.759 | 0.004 | 6.770 |
| hsa-miR-627 | 3 | 4 | −0.071 | 0.907 | 1.050 |
| hsa-miR-628-5p | 4 | 6 | 3.880 | 0.107 | −14.727 |
| hsa-miR-636 | 4 | 2 | 3.932 | 0.000 | −15.259 |
| hsa-miR-642 | 0 | 2 | n/a | n/a | n/a |
| hsa-miR-652 | 3 | 5 | −0.003 | 0.998 | 1.002 |
| hsa-miR-654-3p | 0 | 2 | n/a | n/a | n/a |
| hsa-miR-654-5p | 0 | 2 | n/a | n/a | n/a |
| hsa-miR-655 | 2 | 2 | 2.311 | 0.034 | −4.963 |
| hsa-miR-660 | 4 | 5 | −3.029 | 0.007 | 8.160 |
| hsa-miR-671-3p | 2 | 2 | −0.691 | 0.166 | 1.615 |
| hsa-miR-672 | 2 | 2 | 0.828 | 0.480 | −1.775 |
| hsa-miR-674 | 1 | 0 | n/a | n/a | n/a |
| hsa-miR-708 | 1 | 1 | 0.417 | 0.465 | −1.335 |
| hsa-miR-744 | 2 | 5 | −0.422 | 0.523 | 1.340 |
| hsa-miR-758 | 2 | 2 | 3.177 | 0.000 | −9.046 |
| hsa-miR-872 | 3 | 1 | 8.546 | 0.000 | −373.817 |
| hsa-miR-874 | 1 | 2 | −1.268 | 0.001 | 2.409 |
| hsa-miR-885-5p | 4 | 5 | 0.371 | 0.676 | −1.293 |
| hsa-miR-886-3p | 1 | 1 | −0.273 | 0.843 | 1.208 |
| hsa-miR-886-5p | 3 | 3 | −2.511 | 0.002 | 5.699 |
| hsa-miR-888 | 0 | 1 | n/a | n/a | n/a |
| hsa-miR-889 | 1 | 1 | 0.192 | 0.871 | −1.142 |
| hsa-miR-890 | 0 | 2 | n/a | n/a | n/a |
| hsa-miR-211 | 1 | 0 | n/a | n/a | n/a |
| hsa-miR-212 | 2 | 3 | −2.726 | 0.008 | 6.615 |
| hsa-miR-220 | 1 | 0 | n/a | n/a | n/a |
| hsa-miR-325 | 1 | 0 | n/a | n/a | n/a |
| hsa-miR-346 | 2 | 2 | −0.241 | 0.670 | 1.182 |
| hsa-miR-376c | 3 | 4 | −0.214 | 0.744 | 1.160 |
| hsa-miR-384 | 2 | 3 | 0.399 | 0.578 | −1.318 |
| hsa-miR-492 | 1 | 0 | n/a | n/a | n/a |
| hsa-miR-506 | 2 | 1 | 5.232 | 0.000 | −37.576 |
| hsa-miR-511 | 3 | 1 | 5.480 | 0.001 | −44.631 |
| hsa-miR-517b | 2 | 3 | −1.075 | 0.033 | 2.107 |
| hsa-miR-520b | 2 | 3 | 5.448 | 0.000 | −43.648 |
| dme-miR-7 | 1 | 1 | 0.751 | 0.711 | −1.683 |
| hsa-miR-30a-3p | 3 | 4 | −0.214 | 0.881 | 1.160 |
| hsa-miR-30a-5p | 3 | 3 | −0.958 | 0.391 | 1.943 |
| hsa-miR-30d | 3 | 2 | −0.453 | 0.778 | 1.369 |
| hsa-miR-30e-3p | 4 | 4 | −2.479 | 0.158 | 5.574 |
| hsa-miR-34b | 1 | 0 | n/a | n/a | n/a |
| hsa-miR-126# | 5 | 2 | −5.960 | 0.004 | 62.264 |
| hsa-miR-154# | 3 | 0 | n/a | n/a | n/a |
| U6snRNA | 5 | 5 | −1.095 | 0.589 | 2.137 |
| hsa-miR-206 | 2 | 1 | 0.712 | 0.435 | −1.638 |
| hsa-miR-213 | 1 | 0 | n/a | n/a | n/a |
| hsa-miR-302d | 2 | 0 | n/a | n/a | n/a |
| hsa-miR-378 | 0 | 1 | n/a | n/a | n/a |
| hsa-miR-1257 | 0 | 1 | n/a | n/a | n/a |
| hsa-miR-200a# | 0 | 1 | n/a | n/a | n/a |
| hsa-miR-432 | 2 | 1 | 7.280 | 0.000 | −155.465 |
| hsa-miR-432# | 0 | 1 | n/a | n/a | n/a |
| hsa-miR-497 | 2 | 0 | n/a | n/a | n/a |
| hsa-miR-500 | 1 | 1 | −1.733 | 0.599 | 3.324 |
| hsa-miR-1238 | 1 | 0 | n/a | n/a | n/a |
| hsa-miR-517# | 1 | 0 | n/a | n/a | n/a |
| hsa-miR-516-3p | 0 | 1 | n/a | n/a | n/a |
| rno-miR-7# | 3 | 4 | −2.340 | 0.051 | 5.064 |
| hsa-miR-656 | 3 | 1 | 6.791 | 0.003 | −110.703 |
| hsa-miR-657 | 1 | 0 | n/a | n/a | n/a |
| hsa-miR-552 | 1 | 0 | n/a | n/a | n/a |
| hsa-miR-554 | 1 | 0 | n/a | n/a | n/a |
| hsa-miR-557 | 1 | 0 | n/a | n/a | n/a |
| hsa-miR-562 | 1 | 0 | n/a | n/a | n/a |
| hsa-miR-564 | 1 | 0 | n/a | n/a | n/a |
| hsa-miR-566 | 1 | 1 | −0.068 | 0.941 | 1.048 |
| hsa-miR-569 | 1 | 0 | n/a | n/a | n/a |
| hsa-miR-587 | 1 | 0 | n/a | n/a | n/a |
| hsa-miR-588 | 2 | 0 | n/a | n/a | n/a |
| hsa-miR-589 | 1 | 0 | n/a | n/a | n/a |
| hsa-miR-591 | 1 | 1 | −1.778 | 0.477 | 3.431 |
| hsa-miR-624 | 0 | 1 | n/a | n/a | n/a |
| hsa-miR-601 | 2 | 3 | −3.862 | 0.000 | 14.538 |
| hsa-miR-626 | 1 | 0 | n/a | n/a | n/a |
| hsa-miR-629 | 1 | 1 | −0.367 | 0.773 | 1.290 |
| hsa-miR-630 | 2 | 0 | n/a | n/a | n/a |
| hsa-miR-603 | 1 | 2 | −6.163 | 0.000 | 71.668 |
| hsa-miR-604 | 1 | 0 | n/a | n/a | n/a |
| hsa-miR-606 | 1 | 0 | n/a | n/a | n/a |
| hsa-miR-607 | 1 | 0 | n/a | n/a | n/a |
| hsa-miR-633 | 0 | 1 | n/a | n/a | n/a |
| hsa-miR-637 | 1 | 0 | n/a | n/a | n/a |
| hsa-miR-638 | 1 | 1 | 1.166 | 0.526 | −2.243 |
| hsa-miR-639 | 2 | 1 | 6.003 | 0.002 | −64.132 |
| hsa-miR-613 | 1 | 0 | n/a | n/a | n/a |
| hsa-miR-614 | 1 | 0 | n/a | n/a | n/a |
| hsa-miR-617 | 0 | 1 | n/a | n/a | n/a |
| hsa-miR-644 | 1 | 0 | n/a | n/a | n/a |
| hsa-miR-645 | 3 | 1 | 5.055 | 0.007 | −33.244 |
| hsa-miR-646 | 2 | 0 | n/a | n/a | n/a |
| hsa-miR-650 | 3 | 0 | n/a | n/a | n/a |
| hsa-miR-661 | 1 | 0 | n/a | n/a | n/a |
| hsa-miR-571 | 3 | 2 | −3.986 | 0.022 | 15.842 |
| hsa-miR-572 | 2 | 0 | n/a | n/a | n/a |
| hsa-miR-578 | 1 | 0 | n/a | n/a | n/a |
| hsa-miR-581 | 1 | 0 | n/a | n/a | n/a |
| hsa-miR-583 | 1 | 0 | n/a | n/a | n/a |
| hsa-miR-584 | 0 | 1 | n/a | n/a | n/a |
| hsa-miR-766 | 5 | 5 | −3.372 | 0.032 | 10.355 |
| hsa-miR-595 | 2 | 1 | 1.226 | 0.058 | −2.339 |
| hsa-miR-668 | 1 | 1 | −0.473 | 0.725 | 1.388 |
| hsa-miR-767-5p | 2 | 0 | n/a | n/a | n/a |
| hsa-miR-769-5p | 1 | 1 | −0.350 | 0.619 | 1.275 |
| hsa-miR-770-5p | 2 | 0 | n/a | n/a | n/a |
| hsa-miR-505# | 2 | 1 | 3.574 | 0.026 | −11.906 |
| hsa-miR-218-1# | 1 | 0 | n/a | n/a | n/a |
| hsa-miR-223# | 3 | 4 | −3.003 | 0.006 | 8.018 |
| hsa-miR-136# | 1 | 0 | n/a | n/a | n/a |
| hsa-miR-195# | 1 | 0 | n/a | n/a | n/a |
| hsa-miR-130b# | 1 | 0 | n/a | n/a | n/a |
| hsa-miR-26a-2# | 1 | 0 | n/a | n/a | n/a |
| hsa-miR-361-3p | 1 | 1 | 0.854 | 0.609 | −1.807 |
| hsa-let-7g# | 1 | 0 | n/a | n/a | n/a |
| hsa-miR-374a# | 0 | 1 | n/a | n/a | n/a |
| hsa-miR-23b# | 1 | 0 | n/a | n/a | n/a |
| hsa-miR-376a# | 1 | 0 | n/a | n/a | n/a |
| hsa-miR-130a# | 1 | 0 | n/a | n/a | n/a |
| hsa-miR-148a# | 1 | 0 | n/a | n/a | n/a |
| hsa-miR-33a | 0 | 1 | n/a | n/a | n/a |
| hsa-miR-33a# | 1 | 1 | 0.127 | 0.899 | −1.092 |
| hsa-miR-92a-1# | 1 | 0 | n/a | n/a | n/a |
| hsa-miR-93# | 3 | 5 | −2.910 | 0.008 | 7.517 |
| hsa-miR-96# | 1 | 0 | n/a | n/a | n/a |
| hsa-miR-99a# | 0 | 1 | n/a | n/a | n/a |
| hsa-miR-101# | 2 | 1 | 4.647 | 0.011 | −25.051 |
| hsa-miR-144# | 3 | 3 | −0.465 | 0.687 | 1.380 |
| hsa-miR-145# | 3 | 1 | 3.253 | 0.031 | −9.535 |
| hsa-miR-924 | 1 | 0 | n/a | n/a | n/a |
| hsa-miR-148b# | 0 | 1 | n/a | n/a | n/a |
| hsa-miR-146a# | 1 | 0 | n/a | n/a | n/a |
| hsa-miR-149# | 1 | 1 | −0.820 | 0.408 | 1.766 |
| hsa-miR-29b-2# | 1 | 0 | n/a | n/a | n/a |
| hsa-miR-15b# | 0 | 4 | n/a | n/a | n/a |
| hsa-miR-27b# | 0 | 1 | n/a | n/a | n/a |
| hsa-miR-935 | 1 | 0 | n/a | n/a | n/a |
| hsa-miR-937 | 1 | 0 | n/a | n/a | n/a |
| hsa-miR-335# | 1 | 1 | −1.044 | 0.370 | 2.061 |
| hsa-miR-942 | 3 | 1 | 1.848 | 0.187 | −3.601 |
| hsa-miR-943 | 1 | 0 | n/a | n/a | n/a |
| hsa-miR-99b# | 2 | 0 | n/a | n/a | n/a |
| hsa-miR-541# | 3 | 3 | −3.481 | 0.001 | 11.163 |
| hsa-miR-892b | 1 | 1 | 0.038 | 0.952 | −1.027 |
| hsa-miR-9# | 1 | 1 | 0.111 | 0.944 | −1.080 |
| hsa-miR-151-3p | 5 | 5 | 0.459 | 0.836 | −1.374 |
| hsa-miR-340# | 2 | 1 | 4.329 | 0.022 | −20.098 |
| hsa-miR-190b | 3 | 1 | 5.861 | 0.001 | −58.132 |
| hsa-miR-545# | 1 | 1 | −0.983 | 0.324 | 1.977 |
| hsa-miR-183# | 0 | 1 | n/a | n/a | n/a |
| hsa-miR-192# | 1 | 0 | n/a | n/a | n/a |
| hsa-miR-200c# | 0 | 1 | n/a | n/a | n/a |
| hsa-miR-214# | 0 | 1 | n/a | n/a | n/a |
| hsa-miR-22# | 2 | 2 | −3.365 | 0.001 | 10.302 |
| hsa-miR-30d# | 2 | 0 | n/a | n/a | n/a |
| hsa-miR-424# | 1 | 1 | −0.646 | 0.338 | 1.564 |
| hsa-miR-10b# | 4 | 3 | −3.331 | 0.003 | 10.060 |
| hsa-miR-34a# | 2 | 0 | n/a | n/a | n/a |
| hsa-miR-744# | 2 | 1 | 5.348 | 0.000 | −40.743 |
| hsa-miR-409-3p | 3 | 5 | −4.124 | 0.011 | 17.439 |
| hsa-miR-483-3p | 1 | 0 | n/a | n/a | n/a |
| hsa-miR-543 | 1 | 1 | 0.107 | 0.924 | −1.077 |
| hsa-miR-106b# | 2 | 2 | −2.100 | 0.028 | 4.287 |
| hsa-miR-520c-3p | 3 | 2 | −2.666 | 0.002 | 6.345 |
| hsa-let-7b# | 1 | 0 | n/a | n/a | n/a |
| hsa-let-7f-1# | 1 | 0 | n/a | n/a | n/a |
| hsa-let-7f-2# | 1 | 0 | n/a | n/a | n/a |
| hsa-miR-15a# | 0 | 1 | n/a | n/a | n/a |
| hsa-miR-16-1# | 0 | 1 | n/a | n/a | n/a |
| hsa-miR-17# | 0 | 1 | n/a | n/a | n/a |
| hsa-miR-18a# | 1 | 1 | 0.135 | 0.892 | −1.098 |
| hsa-miR-19b-1# | 3 | 1 | 3.036 | 0.023 | −8.204 |
| hsa-miR-628-3p | 3 | 0 | n/a | n/a | n/a |
| hsa-miR-20a# | 2 | 0 | n/a | n/a | n/a |
| hsa-miR-21# | 0 | 2 | n/a | n/a | n/a |
| hsa-miR-23a# | 2 | 0 | n/a | n/a | n/a |
| hsa-miR-24-2# | 1 | 0 | n/a | n/a | n/a |
| hsa-miR-26a-1# | 1 | 1 | −0.375 | 0.324 | 1.297 |
| hsa-miR-26b# | 2 | 1 | 2.919 | 0.004 | −7.562 |
| hsa-miR-27a# | 2 | 2 | −0.604 | 0.512 | 1.520 |
| hsa-miR-151-5P | 2 | 4 | −1.406 | 0.330 | 2.650 |
| hsa-miR-765 | 0 | 1 | n/a | n/a | n/a |
| hsa-miR-338-5P | 2 | 1 | 1.726 | 0.074 | −3.308 |
| hsa-miR-577 | 1 | 2 | −8.914 | 0.001 | 482.335 |
| hsa-miR-144 | 2 | 2 | −4.241 | 0.002 | 18.903 |
| hsa-miR-590-3P | 3 | 2 | −5.321 | 0.000 | 39.962 |
| hsa-miR-191# | 0 | 1 | n/a | n/a | n/a |
| hsa-miR-520D-3P | 2 | 1 | 2.103 | 0.070 | −4.296 |
| hsa-miR-1224-3P | 1 | 0 | n/a | n/a | n/a |
| has-miR-1305 | 4 | 6 | 1.157 | 0.697 | −2.230 |
| hsa-miR-513C | 2 | 0 | n/a | n/a | n/a |
| hsa-miR-1226# | 1 | 0 | n/a | n/a | n/a |
| hsa-miR-1236 | 1 | 0 | n/a | n/a | n/a |
| hsa-miR-1225-3P | 0 | 1 | n/a | n/a | n/a |
| hsa-miR-1233 | 3 | 1 | 8.820 | 0.000 | −451.910 |
| hsa-miR-1227 | 2 | 0 | n/a | n/a | n/a |
| hsa-miR-1286 | 1 | 0 | n/a | n/a | n/a |
| hsa-miR-1271 | 2 | 2 | −2.938 | 0.003 | 7.665 |
| hsa-miR-1294 | 2 | 0 | n/a | n/a | n/a |
| hsa-miR-1269 | 2 | 0 | n/a | n/a | n/a |
| hsa-miR-1265 | 1 | 1 | −0.553 | 0.671 | 1.467 |
| hsa-miR-1303 | 2 | 0 | n/a | n/a | n/a |
| hsa-miR-1259 | 2 | 0 | n/a | n/a | n/a |
| hsa-miR-1264 | 1 | 0 | n/a | n/a | n/a |
| hsa-miR-1255B | 2 | 3 | −4.258 | 0.004 | 19.137 |
| hsa-miR-1282 | 2 | 0 | n/a | n/a | n/a |
| hsa-miR-1270 | 1 | 0 | n/a | n/a | n/a |
| hsa-miR-548H | 0 | 1 | n/a | n/a | n/a |
| hsa-miR-1254 | 0 | 1 | n/a | n/a | n/a |
| hsa-miR-1251 | 1 | 0 | n/a | n/a | n/a |
| hsa-miR-1292 | 1 | 0 | n/a | n/a | n/a |
| hsa-miR-1182 | 2 | 0 | n/a | n/a | n/a |
| hsa-miR-1288 | 1 | 0 | n/a | n/a | n/a |
| hsa-miR-1291 | 1 | 1 | −0.664 | 0.324 | 1.585 |
| hsa-miR-1275 | 2 | 4 | 0.337 | 0.746 | −1.263 |
| hsa-miR-1183 | 1 | 1 | 0.612 | 0.657 | −1.528 |
| hsa-miR-1276 | 2 | 0 | n/a | n/a | n/a |
| hsa-miR-1180 | 1 | 0 | n/a | n/a | n/a |
| hsa-miR-1243 | 2 | 2 | −3.047 | 0.078 | 8.263 |
| hsa-miR-663B | 1 | 1 | −0.261 | 0.855 | 1.199 |
| hsa-miR-1298 | 1 | 1 | −0.379 | 0.361 | 1.301 |
| hsa-miR-1290 | 4 | 3 | −7.480 | 0.003 | 178.562 |
| hsa-miR-1249 | 1 | 0 | n/a | n/a | n/a |
| hsa-miR-1289 | 1 | 0 | n/a | n/a | n/a |
| hsa-miR-1208 | 2 | 3 | −3.682 | 0.000 | 12.835 |
| hsa-miR-1274A | 3 | 2 | −1.207 | 0.332 | 2.308 |
| hsa-miR-1274B | 6 | 6 | 1.779 | 0.521 | −3.432 |
| hsa-miR-1267 | 4 | 5 | 4.614 | 0.041 | −24.487 |
| hsa-miR-548N | 1 | 0 | n/a | n/a | n/a |
| hsa-miR-1253 | 2 | 1 | 3.837 | 0.004 | −14.295 |
| hsa-miR-1260 | 2 | 2 | 1.683 | 0.059 | −3.211 |
| hsa-miR-1300 | 1 | 1 | −1.052 | 0.651 | 2.074 |
| hsa-miR-1284 | 1 | 0 | n/a | n/a | n/a |
| hsa-miR-1825 | 3 | 3 | −1.817 | 0.039 | 3.524 |
| hsa-miR-1296 | 1 | 0 | n/a | n/a | n/a |

The ΔΔCt values and significance for the differentially regulated miRNAs are also shown.
miRNAs that were previously reported to be significantly different in whole blood from CRPS and control samples are shown in bold at the top of the table.

Sixteen of the 18 miRNAs dysregulated in patients with CRPS from our previous study were detected in human serum-derived exosomes, but only 5 of these (miR-25-3p, miR-320B, miR-939, miR-126-3p, and RNU48) were significantly altered (Table 3). Additionally, the exosomal miRNA signature differed in the directionality of changes compared with that of the whole blood. In the exosomal fraction of patient blood, miR-320B, miR-939, miR-126-3p, and RNU48 were significantly upregulated, whereas they were downregulated in whole blood. Thus hsa-miR-25-3p was the only miRNA exhibiting the same trend in whole blood and exosomes in patients with CRPS. Three LPS-responsive miRNAs in exosomes from RAW 264.7 cells (miR-21-3p, miR-126-3p, and miR-212) were also significantly altered in patients with CRPS. These three exosomal miRNAs were increased both in patients with CRPS and in RAW cells after LPS stimulation. It is expected that analysis of exosomes from a larger sample of patients will indicate that exosomal miRNA are useful biomarkers and/or a secondary strategy for patient stratification.

In summary, the data described herein demonstrate that exosomal content reflects inflammation-induced cellular alterations. Exosomes secreted after LPS treatment show alterations in composition reflective of inflammatory stimulation. Exosomal cytokines, including those elevated in CRPS patients, increase after LPS stimulation. Many exosomal mRNAs that increase after LPS stimulation indicate immune system activation and mediate cytokine signaling pathways. mRNAs encoding CXCL2 (MIP2a), CCL4 (MIP1b) and IL1-Ra are also higher after LPS stimulation. Exosomal miRNAs that are upregulated after LPS stimulation such as miR-155, miR-200c, and miR-146a/b are known to regulate inflammatory mediators.

Moreover, the data described herein demonstrate that exosomes mediate intercellular communication in inflammation and pain. Exosomes derived after LPS treatment induce NFκB activation in naïve cells. Exosomes derived from macrophages are protective in a mouse model of inflammatory pain. Injection of exosomes from LPS-stimulated macrophages into hind paw of CFA treated animals reduces paw edema that is characteristic of CFA model. Macrophage-derived exosomes injected into hind paw of CFA treated animals alleviate thermal hyperalgesia in a TLR-independent mechanism. Exosomal miRNA profile from CRPS patient serum showed that miRNAs altered in this chronic pain state are trafficked by exosomes.

Sequences of the miRNAs described herein are provided at Table 11.

TABLE 11
miRNA Sequences
miRNA sequences were obtained from databases, including miRBase
(www.mirbase.org), NCBI (www.ncbi.nlm.nih.gov), etc.

| miRNA | Accession | | |
|---|---|---|---|
| _ID | _Number | Sequence | SEQ_ID |
| dme- | MI00001 | GAGUGCAUUCCGUAUGGAAGACUAGUGAUUUUGUUGUUUG | SEQ ID |
| miR-7 | 27 | GUCUUUGGUAAUAACAAUAAAUCCCUUGUCUUCUUACGGCG | NO: 1 |
| | | UGCAUUU | |
| hsa-let- | MI00000 | UGGGAUGAGGUAGUAGGUUGUAUAGUUUUAGGGUCACACC | SEQ ID |
| 7a | 60 | CACCACUGGGAGAUAACUAUACAAUCUACUGUCUUUCCUA | NO: 2 |
| hsa-let- | MI00000 | CGGGGUGAGGUAGUAGGUUGUGUGGUUUCAGGGCAGUGAU | SEQ ID |
| 7b | 63 | GUUGCCCCUCGGAAGAUAACUAUACAACCUACUGCCUUCCCU | NO: 3 |
| | | G | |
| hsa-let- | MI00000 | GCAUCCGGGUUGAGGUAGUAGGUUGUAUGGUUUAGAGUUA | SEQ ID |
| 7c | 64 | CACCCUGGGAGUUAACUGUACAACCUUCUAGCUUUCCUUGGA | NO: 4 |
| | | GC | |
| hsa-let- | MI00000 | CCUAGGAAGAGGUAGUAGGUUGCAUAGUUUUAGGGCAGGG | SEQ ID |
| 7d | 65 | AUUUUGCCCACAAGGAGGUAACUAUACGACCUGCUGCCUUUC | NO: 5 |
| | | UUAGG | |
| hsa-let- | MI00000 | CCCGGGCUGAGGUAGGAGGUUGUAUAGUUGAGGAGGACACC | SEQ ID |
| 7e | 66 | CAAGGAGAUCACUAUACGGCCUCCUAGCUUUCCCCAGG | NO: 6 |
| hsa-let- | MI00000 | UCAGAGUGAGGUAGUAGAUUGUAUAGUUGUGGGGUAGUGA | SEQ ID |
| 7f-1 | 67 | UUUUACCCUGUUCAGGAGAUAACUAUACAAUCUAUUGCCUU | NO: 7 |
| | | CCCUGA | |
| hsa-let- | MI00000 | UGUGGGAUGAGGUAGUAGAUUGUAUAGUUUUAGGGUCAUA | SEQ ID |
| 7f-2 | 68 | CCCCAUCUUGGAGAUAACUAUACAGUCUACUGUCUUUCCCAC | NO: 8 |
| | | G | |
| hsa-let- | MI00004 | AGGCUGAGGUAGUAGUUUGUACAGUUUGAGGGUCUAUGAU | SEQ ID |
| 7g | 33 | ACCACCCGGUACAGGAGAUAACUGUACAGGCCACUGCCUUGC | NO: 9 |
| | | CA | |
| hsa-let- | M100004 | CUGGCUGAGGUAGUAGUUUGUGCUGUUGGUCGGGUUGUGA | SEQ ID |
| 7i | 34 | CAUUGCCCGCUGUGGAGAUAACUGCGCAAGCUACUGCCUUGC | NO: 10 |
| | | UA | |
| hsa- | MIMAT00 | UGGAAUGUAAAGAAGUAUGUAU | SEQ ID |
| miR-1 | 00416 | | NO: 11 |
| hsa- | MI00001 | CCUGUUGCCACAAACCCGUAGAUCCGAACUUGUGGUAUUAG | SEQ ID |
| miR- | 02 | UCCGCACAAGCUUGUAUCUAUAGGUAUGUGUCUGUUAGG | NO: 12 |
| 100 | | | |
| hsa- | MI00001 | UGCCCUGGCUCAGUUAUCACAGUGCUGAUGCUGUCUAUUCU | SEQ ID |
| miR- | 03 | AAAGGUACAGUACUGUGAUAACUGAAGGAUGGCA | NO: 13 |
| 101 | | | |
| hsa- | MIMAT00 | AGCAGCAUUGUACAGGGCUAUGA | SEQ ID |
| miR- | 00101 | | NO: 14 |
| 103 | | | |
| hsa- | MI00001 | CCUUGGCCAUGUAAAAGUGCUUACAGUGCAGGUAGCUUUUU | SEQ ID |
| miR- | 13 | GAGAUCUACUGCAAUGUAAGCACUUCUUACAUUACCAUGG | NO: 15 |
| 106a | | | |
| hsa- | MI00007 | CCUGCCGGGGCUAAAGUGCUGACAGUGCAGAUAGUGGUCCU | SEQ ID |
| miR- | 34 | CUCCGUGCUACCGCACUGUGGGUACUUGCUGCUCCAGCAGG | NO: 16 |
| 106b | | | |
| hsa- | MIMAT00 | AGCAGCAUUGUACAGGGCUAUCA | SEQ ID |
| miR- | 00104 | | NO: 17 |
| 107 | | | |
| hsa- | MI00002 | GAUCUGUCUGUCUUCUGUAUAUACCCUGUAGAUCCGAAUUU | SEQ ID |
| miR- | 66 | GUGUAAGGAAUUUUGUGGUCACAAAUUCGUAUCUAGGGGAA | NO: 18 |
| 10a | | UAUGUAGUUGACAUAAACACUCCGCUCU | |
| hsa- | MI00002 | CCAGAGGUUGUAACGUUGUCUAUAUAUACCCUGUAGAACCG | SEQ ID |
| miR- | 67 | AAUUUGUGUGGUAUCCGUAUAGUCACAGAUUCGAUUCUAGG | NO: 19 |
| 10b | | GGAAUAUAUGGUCGAUGCAAAAACUUCA | |
| hsa- | MI00062 | GCUGCUGGACCCACCCGGCCGGGAAUAGUGCUCCUGGUUGU | SEQ ID |
| miR- | 73 | UUCCGGCUCGCGUGGGUGUGUCGGCGGC | NO: 20 |
| 1180 | | | |
| hsa- | MIMAT00 | GAGGGUCUUGGGAGGGAUGUGAC | SEQ ID |
| miR- | 05827 | | NO: 21 |
| 1182 | | | |
| hsa- | MIMAT00 | CACUGUAGGUGAUGGUGAGAGUGGGCA | SEQ ID |
| miR- | 05828 | | NO: 22 |
| 1183 | | | |
| hsa- | MIMAT00 | UCACUGUUCAGACAGGCGGA | SEQ ID |
| miR- | 05873 | | NO: 23 |
| 1208 | | | |
| hsa- | MI00004 | CCUUAGCAGAGCUGUGGAGUGUGACAAUGGUGUUUGUGUC | SEQ ID |
| miR- | 42 | UAAACUAUCAAACGCCAUUAUCACACUAAAUAGCUACUGCUA | NO: 24 |
| 122 | | GGC | |
| hsa- | MIMAT00 | CCCCACCUCCUCUCUCCUCAG | SEQ ID |
| miR- | 05459 | | NO: 25 |
| 1224- | | | |
| 3p | | | |
| hsa- | MIMAT00 | UGAGCCCCUGUGCCGCCCCCAG | SEQ ID |
| miR- | 05573 | | NO: 26 |
| 1225- | | | |
| 3p | | | |
| hsa- | MI00063 | GUGAGGGCAUGCAGGCCUGGAUGGGGCAGCUGGGAUGGUCC | SEQ ID |
| miR- | 13 | AAAAGGGUGGCCUCACCAGCCCUGUGUUCCCUAG | NO: 27 |
| 1226 | | | |
| hsa- | MI00063 | GUGGGGCCAGGCGGUGGUGGGCACUGCUGGGGUGGGCACAG | SEQ ID |
| miR- | 16 | CAGCCAUGCAGAGCGGGCAUUUGACCCCGUGCCACCCUUUUC | NO: 28 |
| 1227 | | CCCAG | |
| hsa- | MI00063 | GUGAGUGGGAGGCCAGGGCACGGCAGGGGGAGCUGCAGGGC | SEQ ID |
| miR- | 23 | UAUGGGAGGGGCCCCAGCGUCUGAGCCCUGUCCUCCCGCAG | NO: 29 |
| 1233 | | | |
| hsa- | MI00063 | GUGAGUGACAGGGGAAAUGGGGAUGGACUGGAAGUGGGCA | SEQ ID |
| miR- | 26 | GCAUGGAGCUGACCUUCAUCAUGGCUUGGCCAACAUAAUGCC | NO: 30 |
| 1236 | | UCUUCCCCUUGUCUCUCCAG | |
| hsa- | MI00063 | GUGAGUGGGAGCCCCAGUGUGUGGUUGGGGCCAUGGCGGG | SEQ ID |
| miR- | 28 | UGGGCAGCCCAGCCUCUGAGCCUUCCUCGUCUGUCUGCCCCA | NO: 31 |
| 1238 | | G | |
| hsa- | MI00004 | AGGCCUCUCUCUCCGUGUUCACAGCGGACCUUGAUUUAAAU | SEQ ID |
| miR- | 43 | GUCCAUACAAUUAAGGCACGCGGUGAAUGCCAAGAAUGGGG | NO: 32 |
| 124 | | CUG | |
| hsa- | MIMAT00 | AACUGGAUCAAUUAUAGGAGUG | SEQ ID |
| miR- | 05894 | | NO: 33 |
| 1243 | | | |
| hsa- | MIMAT00 | ACGCCCUUCCCCCCCUUCUUCA | SEQ ID |
| miR- | 05901 | | NO: 34 |
| 1249 | | | |
| hsa- | MI00063 | GUGGACUCUAGCUGCCAAAGGCGCUUCUCCUUCUGAACAGAG | SEQ ID |
| miR- | 86 | CGCUUUGCUCAGCCAGUGUAGACAUGGC | NO: 35 |
| 1251 | | | |
| hsa- | MIMAT00 | AGAGAAGAAGAUCAGCCUGCA | SEQ ID |
| miR- | 05904 | | NO: 36 |
| 1253 | | | |
| hsa- | MIMAT00 | AGCCUGGAAGCUGGAGCCUGCAGU | SEQ ID |
| miR- | 05905 | | NO: 37 |
| 1254 | | | |
| hsa- | MI00064 | UACGGAUGAGCAAAGAAAGUGGUUUCUUAAAAUGGAAUCUA | SEQ ID |
| miR- | 35 | CUCUUUGUGAAGAUGCUGUGAA | NO: 38 |
| 1255b | | | |
| hsa- | MIMAT00 | AGUGAAUGAUGGGUUCUGACC | SEQ ID |
| miR- | 05908 | | NO: 39 |
| 1257 | | | |
| hsa- | MI00063 | GCCUUUGCAGCUGAUGAUACAGCUUCUUUCCCCAUCAGAUCG | SEQ ID |
| miR- | 93 | ACCCUGUUGAUCUCUACACUAUUGGCCAGUUUUGUCUGAUG | NO: 40 |
| 1259 | | CAUUGGC | |
| hsa- | MIMAT00 | ACAGGUGAGGUUCUUGGGAGCC | SEQ ID |
| miR- | 04602 | | NO: 41 |
| 125a- | | | |
| 3p | | | |
| hsa- | MIMAT00 | UCCCUGAGACCCUUUAACCUGUGA | SEQ ID |
| miR- | 00443 | | NO: 42 |
| 125a- | | | |
| 5p | | | |
| hsa- | MI00004 | UGCGCUCCUCUCAGUCCCUGAGACCCUAACUUGUGAUGUUU | SEQ ID |
| miR- | 46 | ACCGUUUAAAUCCACGGGUUAGGCUCUUGGGAGCUGCGAGU | NO: 43 |
| 125b | | CGUGCU | |
| hsa- | MI00004 | CGCUGGCGACGGGACAUUAUUACUUUUGGUACGCGCUGUGA | SEQ ID |
| miR- | 71 | CACUUCAAACUCGUACCGUGAGUAAUAAUGCGCCGUCCACGG | NO: 44 |
| 126 | | CA | |
| hsa- | MIMAT00 | AUCCCACCUCUGCCACCA | SEQ ID |
| miR- | 05911 | | NO: 45 |
| 1260 | | | |
| hsa- | MIMAT00 | CAAGUCUUAUUUGAGCACCUGUU | SEQ ID |
| miR- | 05791 | | NO: 46 |
| 1264 | | | |
| hsa- | MIMAT00 | CAGGAUGUGGUCAAGUGUUGUU | SEQ ID |
| miR- | 05918 | | NO: 47 |
| 1265 | | | |
| hsa- | MIMAT00 | CCUGUUGAAGUGUAAUCCCCA | SEQ ID |
| miR- | 05921 | | NO: 48 |
| 1267 | | | |
| hsa- | MIMAT00 | CUGGACUGAGCCGUGCUACUGG | SEQ ID |
| miR- | 05923 | | NO: 49 |
| 1269 | | | |
| hsa- | MIMAT00 | CGGAUCCGUCUGAGCUUGGCU | SEQ ID |
| miR- | 00446 | | NO: 50 |
| 127-3p | | | |
| hsa- | MIMAT00 | CUGAAGCUCAGAGGGCUCUGAU | SEQ ID |
| miR- | 04604 | | NO: 51 |
| 127-5p | | | |
| hsa- | MIMAT00 | CUGGAGAUAUGGAAGAGCUGUGU | SEQ ID |
| miR- | 05924 | | NO: 52 |
| 1270 | | | |
| hsa- | MI00038 | CACCCAGAUCAGUGCUUGGCACCUAGCAAGCACUCAGUAAAU | SEQ ID |
| miR- | 14 | AUUUGUUGAGUGCCUGCUAUGUGCCAGGCAUUGUGCUGAG | NO: 53 |
| 1271 | | GGCU | |
| hsa- | MIMAT00 | GUCCCUGUUCAGGCGCCA | SEQ ID |
| miR- | 05927 | | NO: 54 |
| 1274a | | | |
| hsa- | MIMAT00 | UCCCUGUUCGGGCGCCA | SEQ ID |
| miR- | 05938 | | NO: 55 |
| 1274b | | | |
| hsa- | MIMAT00 | GUGGGGGAGAGGCUGUC | SEQ ID |
| miR- | 05929 | | NO: 56 |
| 1275 | | | |
| hsa- | MIMAT00 | UAAAGAGCCCUGUGGAGACA | SEQ ID |
| miR- | 05930 | | NO: 57 |
| 1276 | | | |
| hsa- | MI00004 | UGAGCUGUUGGAUUCGGGGCCGUAGCACUGUCUGAGAGGUU | SEQ ID |
| miR- | 47 | UACAUUUCUCACAGUGAACCGGUCUCUUUUUCAGCUGCUUC | NO: 58 |
| 128 | | | |
| hsa- | MIMAT00 | UCGUUUGCCUUUUUCUGCUU | SEQ ID |
| miR- | 05940 | | NO: 59 |
| 1282 | | | |
| hsa- | MIMAT00 | UCUAUACAGACCCUGGCUUUUC | SEQ ID |
| miR- | 05941 | | NO: 60 |
| 1284 | | | |
| hsa- | MIMAT00 | UGCAGGACCAAGAUGAGCCCU | SEQ ID |
| miR- | 05877 | | NO: 61 |
| 1286 | | | |
| hsa- | MI00064 | GAGGGUGUUGAUCAGCAGAUCAGGACUGUAACUCACCAUAG | SEQ ID |
| miR- | 32 | UGGUGGACUGCCCUGAUCUGGAGACCACUGCCUU | NO: 62 |
| 1288 | | | |
| hsa- | MIMAT00 | UGGAGUCCAGGAAUCUGCAUUUU | SEQ ID |
| miR- | 05879 | | NO: 63 |
| 1289 | | | |
| hsa- | MIMAT00 | CUUUUUGCGGUCUGGGCUUGC | SEQ ID |
| miR- | 00242 | | NO: 64 |
| 129-5p | | | |
| hsa- | MIMAT00 | UGGAUUUUUGGAUCAGGGA | SEQ ID |
| miR- | 05880 | | NO: 65 |
| 1290 | | | |
| hsa- | MIMAT00 | UGGCCCUGACUGAAGACCAGCAGU | SEQ ID |
| miR- | 05881 | | NO: 66 |
| 1291 | | | |
| hsa- | MI00064 | CCUGGGAACGGGUUCCGGCAGACGCUGAGGUUGCGUUGACG | SEQ ID |
| miR- | 33 | CUCGCGCCCCGGCUCCCGUUCCAGG | NO: 67 |
| 1292 | | | |
| hsa- | MIMAT00 | UGUGAGGUUGGCAUUGUUGUCU | SEQ ID |
| miR- | 05884 | | NO: 68 |
| 1294 | | | |
| hsa- | MI00037 | ACCUACCUAACUGGGUUAGGGCCCUGGCUCCAUCUCCUUUAG | SEQ ID |
| miR- | 80 | GAAAACCUUCUGUGGGGAGUGGGGCUUCGACCCUAACCCAG | NO: 69 |
| 1296 | | GUGGGCUGU | |
| hsa- | MI00039 | AGACGAGGAGUUAAGAGUUCAUUCGGCUGUCCAGAUGUAUC | SEQ ID |
| miR- | 38 | CAAGUACCCUGUGUUAUUUGGCAAUAAAUACAUCUGGGCAA | NO: 70 |
| 1298 | | CUGACUGAACUUUUCACUUUUCAUGACUCA | |
| hsa- | MIMAT00 | UUGAGAAGGAGGCUGCUG | SEQ ID |
| miR- | 05888 | | NO: 71 |
| 1300 | | | |
| hsa- | MIMAT00 | UUUAGAGACGGGGUCUUGCUCU | SEQ ID |
| miR- | 05891 | | NO: 72 |
| 1303 | | | |
| hsa- | MIMAT00 | UUUUCAACUCUAAUGGGAGAGA | SEQ ID |
| miR- | 05893 | | NO: 73 |
| 1305 | | | |
| hsa- | MI00004 | UGCUGCUGGCCAGAGCUCUUUUCACAUUGUGCUACUGUCUG | SEQ ID |
| miR- | 48 | CACCUGUCACUAGCAGUGCAAUGUUAAAAGGGCAUUGGCCG | NO: 74 |
| 130a | | UGUAGUG | |
| hsa- | MI00007 | GGCCUGCCCGACACUCUUUCCCUGUUGCACUACUAUAGGCCG | SEQ ID |
| miR- | 48 | CUGGGAAGCAGUGCAAUGAUGAAAGGGCAUCGGUCAGGUC | NO: 75 |
| 130b | | | |
| hsa- | MI00004 | CCGCCCCCGCGUCUCCAGGGCAACCGUGGCUUUCGAUUGUUA | SEQ ID |
| miR- | 49 | CUGUGGGAACUGGAGGUAACAGUCUACAGCCAUGGUCGCCCC | NO: 76 |
| 132 | | GCAGCACGCCCACGCGC | |
| hsa- | MI00004 | ACAAUGCUUUGCUAGAGCUGGUAAAAUGGAACCAAAUCGCCU | SEQ ID |
| miR- | 50 | CUUCAAUGGAUUUGGUCCCCUUCAACCAGCUGUAGCUAUGCA | NO: 77 |
| 133a | | UUGA | |
| hsa- | MIMAT00 | UUUGGUCCCCUUCAACCAGCUA | SEQ ID |
| miR- | 00770 | | NO: 78 |
| 133b | | | |
| hsa- | MI00004 | CAGGGUGUGUGACUGGUUGACCAGAGGGGCAUGCACUGUGU | SEQ ID |
| miR- | 74 | UCACCCUGUGGGCCACCUAGUCACCAACCCUC | NO: 79 |
| 134 | | | |
| hsa- | MI00004 | AGGCCUCGCUGUUCUCUAUGGCUUUUUAUUCCUAUGUGAUU | SEQ ID |
| miR- | 52 | CUACUGCUCACUCAUAUAGGGAUUGGAGCCGUGGCGCACGGC | NO: 80 |
| 135a | | GGGGACA | |
| hsa- | MI00008 | CACUCUGCUGUGGCCUAUGGCUUUUCAUUCCUAUGUGAUUG | SEQ ID |
| miR- | 10 | CUGUCCCAAACUCAUGUAGGGCUAAAAGCCAUGGGCUACAGU | NO: 81 |
| 135b | | GAGGGGCGAGCUCC | |
| hsa- | MI00004 | UGAGCCCUCGGAGGACUCCAUUUGUUUUGAUGAUGGAUUCU | SEQ ID |
| miR- | 75 | UAUGCUCCAUCAUCGUCUCAAAUGAGUCUUCAGAGGGUUCU | NO: 82 |
| 136 | | | |
| hsa- | MIMAT00 | UGGAGACGCGGCCCUGUUGGAGU | SEQ ID |
| miR- | 04552 | | NO: 83 |
| 139-3p | | | |
| hsa- | MIMAT00 | UCUACAGUGCACGUGUCUCCAGU | SEQ ID |
| miR- | 00250 | | NO: 84 |
| 139-5p | | | |
| hsa- | MIMAT00 | UACCACAGGGUAGAACCACGG | SEQ ID |
| miR- | 04597 | | NO: 85 |
| 140-3p | | | |
| hsa- | MIMAT00 | CAGUGGUUUUACCCUAUGGUAG | SEQ ID |
| miR- | 00431 | | NO: 86 |
| 140-5p | | | |
| hsa- | MI00004 | CGGCCGGCCCUGGGUCCAUCUUCCAGUACAGUGUUGGAUGG | SEQ ID |
| miR- | 57 | UCUAAUUGUGAAGCUCCUAACACUGUCUGGUAAAGAUGGCU | NO: 87 |
| 141 | | CCCGGGUGGGUUC | |
| hsa- | MIMAT00 | UGUAGUGUUUCCUACUUUAUGGA | SEQ ID |
| miR- | 00434 | | NO: 88 |
| 142-3p | | | |
| hsa- | MIMAT00 | CAUAAAGUAGAAAGCACUACU | SEQ ID |
| miR- | 00433 | | NO: 89 |
| 142-5p | | | |
| hsa- | MI00004 | GCGCAGCGCCCUGUCUCCCAGCCUGAGGUGCAGUGCUGCAUC | SEQ ID |
| miR- | 59 | UCUGGUCAGUUGGGAGUCUGAGAUGAAGCACUGUAGCUCAG | NO: 90 |
| 143 | | GAAGAGAGAAGUUGUUCUGCAGC | |
| hsa- | MI00004 | UGGGGCCCUGGCUGGGAUAUCAUCAUAUACUGUAAGUUUGC | SEQ ID |
| miR- | 60 | GAUGAGACACUACAGUAUAGAUGAUGUACUAGUCCGGGCAC | NO: 91 |
| 144 | | CCCC | |
| hsa- | MI00004 | CACCUUGUCCUCACGGUCCAGUUUUCCCAGGAAUCCCUUAGA | SEQ ID |
| miR- | 61 | UGCUAAGAUGGGGAUUCCUGGAAAUACUGUUCUUGAGGUCA | NO: 92 |
| 145 | | UGGUU | |
| hsa- | MI00004 | CCGAUGUGUAUCCUCAGCUUUGAGAACUGAAUUCCAUGGGU | SEQ ID |
| miR- | 77 | UGUGUCAGUGUCAGACCUCUGAAAUUCAGUUCUUCAGCUGG | NO: 93 |
| 146a | | GAUAUCUCUGUCAUCGU | |
| hsa- | MIMAT00 | UGCCCUGUGGACUCAGUUCUGG | SEQ ID |
| miR- | 04766 | | NO: 94 |
| 146b- | | | |
| 3p | | | |
| hsa- | MIMAT00 | UGAGAACUGAAUUCCAUAGGCU | SEQ ID |
| miR- | 02809 | | NO: 95 |
| 146b- | | | |
| 5p | | | |
| hsa- | MIMAT00 | GUGUGCGGAAAUGCUUCUGCUA | SEQ ID |
| miR- | 04928 | | NO: 96 |
| 147b | | | |
| hsa- | MI00002 | GAGGCAAAGUUCUGAGACACUCCGACUCUGAGUAUGAUAGA | SEQ ID |
| miR- | 53 | AGUCAGUGCACUACAGAACUUUGUCUC | NO: 97 |
| 148a | | | |
| hsa- | MI00008 | CAAGCACGAUUAGCAUUUGAGGUGAAGUUCUGUUAUACACU | SEQ ID |
| miR- | 11 | CAGGCUGUGGCUCUCUGAAAGUCAGUGCAUCACAGAACUUU | NO: 98 |
| 148b | | GUCUCGAAAGCUUUCUA | |
| hsa- | MI00004 | GCCGGCGCCCGAGCUCUGGCUCCGUGUCUUCACUCCCGUGCU | SEQ ID |
| miR- | 78 | UGUCCGAGGAGGGAGGGAGGGACGGGGGCUGUGCUGGGGC | NO: 99 |
| 149 | | AGCUGGA | |
| hsa- | MI00004 | CUCCCCAUGGCCCUGUCUCCCAACCCUUGUACCAGUGCUGGG | SEQ ID |
| miR- | 79 | CUCAGACCCUGGUACAGGCCUGGGGGACAGGGACCUGGGGAC | NO: 100 |
| 150 | | | |
| hsa- | MIMAT00 | CUAGACUGAAGCUCCUUGAGG | SEQ ID |
| miR- | 00757 | | NO: 101 |
| 151-3p | | | |
| hsa- | MIMAT00 | UCGAGGAGCUCACAGUCUAGU | SEQ ID |
| miR- | 04697 | | NO: 102 |
| 151-5p | | | |
| hsa- | MI00004 | UGUCCCCCCCGGCCCAGGUUCUGUGAUACACUCCGACUCGGG | SEQ ID |
| miR- | 62 | CUCUGGAGCAGUCAGUGCAUGACAGAACUUGGGCCCGGAAG | NO: 103 |
| 152 | | GACC | |
| hsa- | MI00004 | GUGGUACUUGAAGAUAGGUUAUCCGUGUUGCCUUCGCUUUA | SEQ ID |
| miR- | 80 | UUUGUGACGAAUCAUACACGGUUGACCUAUUUUUCAGUACC | NO: 104 |
| 154 | | AA | |
| hsa- | MI00006 | CUGUUAAUGCUAAUCGUGAUAGGGGUUUUUGCCUCCAACUG | SEQ ID |
| miR- | 81 | ACUCCUACAUAUUAGCAUUAACAG | NO: 105 |
| 155 | | | |
| hsa- | MI00000 | CCUUGGAGUAAAGUAGCAGCACAUAAUGGUUUGUGGAUUUU | SEQ ID |
| miR- | 69 | GAAAAGGUGCAGGCCAUAUUGUGCUGCCUCAAAAAUACAAG | NO: 106 |
| 15a | | G | |
| hsa- | MI00004 | UUGAGGCCUUAAAGUACUGUAGCAGCACAUCAUGGUUUACA | SEQ ID |
| miR- | 38 | UGCUACAGUCAAGAUGCGAAUCAUUAUUUGCUGCUCUAGAA | NO: 107 |
| 15b | | AUUUAAGGAAAUUCAU | |
| hsa- | MI00000 | GUCAGCAGUGCCUUAGCAGCACGUAAAUAUUGGCGUUAAGA | SEQ ID |
| miR-16 | 70 | UUCUAAAAUUAUCUCCAGUAUUAACUGUGCUGCUGAAGUAA | NO: 108 |
| | | GGUUGAC | |
| hsa- | MI00000 | GUCAGAAUAAUGUCAAAGUGCUUACAGUGCAGGUAGUGAUA | SEQ ID |
| miR-17 | 71 | UGUGCAUCUACUGCAGUGAAGGCACUUGUAGCAUUAUGGUG | NO: 109 |
| AC | | | |
| hsa- | MI00002 | UGAGUUUUGAGGUUGCUUCAGUGAACAUUCAACGCUGUCGG | SEQ ID |
| miR- | 89 | UGAGUUUGGAAUUAAAAUCAAAACCAUCGACCGUUGAUUGU | NO: 110 |
| 181a | | ACCCUAUGGCUAACCAUCAUCUACUCCA | |
| hsa- | MI00002 | AGAAGGGCUAUCAGGCCAGCCUUCAGAGGACUCCAAGGAACA | SEQ ID |
| miR- | 69 | UUCAACGCUGUCGGUGAGUUUGGGAUUUGAAAAAACCACUG | NO: 111 |
| 181a-2 | | ACCGUUGACUGUACCUUGGGGUCCUUA | |
| hsa- | MI00002 | CGGAAAAUUUGCCAAGGGUUUGGGGGAACAUUCAACCUGUC | SEQ ID |
| miR- | 71 | GGUGAGUUUGGGCAGCUCAGGCAAACCAUCGACCGUUGAGU | NO: 112 |
| 181c | | GGACCCUGAGGCCUGGAAUUGCCAUCCU | |
| hsa- | MI00002 | GAGCUGCUUGCCUCCCCCCGUUUUUGGCAAUGGUAGAACUCA | SEQ ID |
| miR- | 72 | CACUGGUGAGGUAACAGGAUCCGGUGGUUCUAGACUUGCCA | NO: 113 |
| 182 | | ACUAUGGGGCGAGGACUCAGCCGGCAC | |
| hsa- | MIMAT00 | UCCAGUGCCCUCCUCUCC | SEQ ID |
| miR- | 06765 | | NO: 114 |
| 1825 | | | |
| hsa- | MI00002 | CCGCAGAGUGUGACUCCUGUUCUGUGUAUGGCACUGGUAGA | SEQ ID |
| miR- | 73 | AUUCACUGUGAACAGUCUCAGUCAGUGAAUUACCGAAGGGCC | NO: 115 |
| 183 | | AUAAACAGAGCAGAGACAGAUCCACGA | |
| hsa- | MIMAT00 | UGGACGGAGAACUGAUAAGGGU | SEQ ID |
| miR- | 00454 | | NO: 116 |
| 184 | | | |
| hsa- | MI00004 | AGGGGGCGAGGGAUUGGAGAGAAAGGCAGUUCCUGAUGGUC | SEQ ID |
| miR- | 82 | CCCUCCCCAGGGGCUGGCUUUCCUCUGGUCCUUCCCUCCCA | NO: 117 |
| 185 | | | |
| hsa- | MI00004 | UGCUUGUAACUUUCCAAAGAAUUCUCCUUUUGGGCUUUCUG | SEQ ID |
| miR- | 83 | GUUUUAUUUUAAGCCCAAAGGUGAAUUUUUUGGGAAGUUU | NO: 118 |
| 186 | | GAGCU | |
| hsa- | MI00002 | GGUCGGGCUCACCAUGACACAGUGUGAGACCUCGGGCUACAA | SEQ ID |
| miR- | 74 | CACAGGACCCGGGCGCUGCUCUGACCCCUCGUGUCUUGUGUU | NO: 119 |
| 187 | | GCAGCCGGAGGGACGCAGGUCCGCA | |
| hsa- | MIMAT00 | CUCCCACAUGCAGGGUUUGCA | SEQ ID |
| miR- | 04613 | | NO: 120 |
| 188-3p | | | |
| hsa- | MIMAT00 | UGCCUACUGAGCUGAUAUCAGU | SEQ ID |
| miR- | 00079 | | NO: 121 |
| 189 | | | |
| hsa- | MI00000 | UGUUCUAAGGUGCAUCUAGUGCAGAUAGUGAAGUAGAUUAG | SEQ ID |
| miR- | 72 | CAUCUACUGCCCUAAGUGCUCCUUCUGGCA | NO: 122 |
| 18a | | | |
| hsa- | MI00015 | UGUGUUAAGGUGCAUCUAGUGCAGUUAGUGAAGCAGCUUAG | SEQ ID |
| miR- | 18 | AAUCUACUGCCCUAAAUGCCCCUUCUGGCA | NO: 123 |
| 18b | | | |
| hsa- | MI00004 | UGCAGGCCUCUGUGUGAUAUGUUUGAUAUAUUAGGUUGUU | SEQ ID |
| miR- | 86 | AUUUAAUCCAACUAUAUAUCAAACAUAUUCCUACAGUGUCU | NO: 124 |
| 190 | | UGCC | |
| hsa- | MIMAT00 | UGAUAUGUUUGAUAUUGGGUU | SEQ ID |
| miR- | 04929 | | NO: 125 |
| 190b | | | |
| hsa- | MI00004 | CGGCUGGACAGCGGGCAACGGAAUCCCAAAAGCAGCUGUUGU | SEQ ID |
| miR- | 65 | CUCCAGAGCAUUCCAGCUGCGCUUGGAUUUCGUCCCCUGCUC | NO: 126 |
| 191 | | UCCUGCCU | |
| hsa- | MI00002 | GCCGAGACCGAGUGCACAGGGCUCUGACCUAUGAAUUGACAG | SEQ ID |
| miR- | 34 | CCAGUGCUCUCGUCUCCCCUCUGGCUGCCAAUUCCAUAGGUC | NO: 127 |
| 192 | | ACAGGUAUGUUCGCCUCAAUGCCAGC | |
| hsa- | MIMAT00 | AACUGGCCUACAAAGUCCCAGU | SEQ ID |
| miR- | 00459 | | NO: 128 |
| 193a- | | | |
| 3p | | | |
| hsa- | MIMAT00 | UGGGUCUUUGCGGGCGAGAUGA | SEQ ID |
| miR- | 04614 | | NO: 129 |
| 193a- | | | |
| 5p | | | |
| hsa- | MI00031 | GUGGUCUCAGAAUCGGGGUUUUGAGGGCGAGAUGAGUUUA | SEQ ID |
| miR- | 37 | UGUUUUAUCCAACUGGCCCUCAAAGUCCCGCUUUUGGGGUC | NO: 130 |
| 193b | | AU | |
| hsa- | MI00004 | AUGGUGUUAUCAAGUGUAACAGCAACUCCAUGUGGACUGUG | SEQ ID |
| miR- | 88 | UACCAAUUUCCAGUGGAGAUGCUGUUACUUUUGAUGGUUAC | NO: 131 |
| 194 | | CAA | |
| hsa- | MI00004 | AGCUUCCCUGGCUCUAGCAGCACAGAAAUAUUGGCACAGGGA | SEQ ID |
| miR- | 89 | AGCGAGUCUGCCAAUAUUGGCUGUGCUGCUCCAGGCAGGGU | NO: 132 |
| 195 | | GGUG | |
| hsa- | MI00011 | ACUGGUCGGUGAUUUAGGUAGUUUCCUGUUGUUGGGAUCC | SEQ ID |
| miR- | 50 | ACCUUUCUCUCGACAGCACGACACUGCCUUCAUUACUUCAGU | NO: 133 |
| 196b | | UG | |
| hsa- | MI00002 | GGCUGUGCCGGGUAGAGAGGGCAGUGGGAGGUAAGAGCUCU | SEQ ID |
| miR- | 39 | UCACCCUUCACCACCUUCUCCACCCAGCAUGGCC | NO: 134 |
| 197 | | | |
| hsa- | MIMAT00 | ACAGUAGUCUGCACAUUGGUUA | SEQ ID |
| miR- | 00232 | | NO: 135 |
| 199a- | | | |
| 3p | | | |
| hsa- | MIMAT00 | CCCAGUGUUCAGACUACCUGUUC | SEQ ID |
| miR- | 00231 | | NO: 136 |
| 199a- | | | |
| 5p | | | |
| hsa- | MIMAT00 | CCCAGUGUUUAGACUAUCUGUUC | SEQ ID |
| miR- | 00263 | | NO: 137 |
| 199b- | | | |
| 5p | | | |
| hsa- | MI00000 | GCAGUCCUCUGUUAGUUUUGCAUAGUUGCACUACAAGAAGA | SEQ ID |
| miR- | 73 | AUGUAGUUGUGCAAAUCUAUGCAAAACUGAUGGUGGCCUGC | NO: 138 |
| 19a | | | |
| hsa- | MI00000 | CACUGUUCUAUGGUUAGUUUUGCAGGUUUGCAUCCAGCUGU | SEQ ID |
| miR- | 74 | GUGAUAUUCUGCUGUGCAAAUCCAUGCAAAACUGACUGUGG | NO: 139 |
| 19b | | UAGUG | |
| hsa- | MI00007 | CCGGGCCCCUGUGAGCAUCUUACCGGACAGUGCUGGAUUUCC | SEQ ID |
| miR- | 37 | CAGCUUGACUCUAACACUGUCUGGUAACGAUGUUCAAAGGU | NO: 140 |
| 200a | | GACCCGC | |
| hsa- | MI00003 | CCAGCUCGGGCAGCCGUGGCCAUCUUACUGGGCAGCAUUGGA | SEQ ID |
| miR- | 42 | UGGAGUCAGGUCUCUAAUACUGCCUGGUAAUGAUGACGGCG | NO: 141 |
| 200b | | GAGCCCUGCACG | |
| hsa- | MI00006 | CCCUCGUCUUACCCAGCAGUGUUUGGGUGCGGUUGGGAGUC | SEQ ID |
| miR- | 50 | UCUAAUACUGCCGGGUAAUGAUGGAGG | NO: 142 |
| 200c | | | |
| hsa- | MI00031 | CGCCUCAGAGCCGCCCGCCGUUCCUUUUUCCUAUGCAUAUAC | SEQ ID |
| miR- | 30 | UUCUUUGAGGAUCUGGCCUAAAGAGGUAUAGGGCAUGGGAA | NO: 143 |
| 202 | | AACGGGGCGGUCGGGUCCUCCCCAGCG | |
| hsa- | MI00002 | GGCUACAGUCUUUCUUCAUGUGACUCGUGGACUUCCCUUUG | SEQ ID |
| miR- | 84 | UCAUCCUAUGCCUGAGAAUAUAUGAAGGAGGCUGGGAAGGC | NO: 144 |
| 204 | | AAAGGGACGUUCAAUUGUCAUCACUGGC | |
| hsa- | MI00002 | AAAGAUCCUCAGACAAUCCAUGUGCUUCUCUUGUCCUUCAUU | SEQ ID |
| miR- | 85 | CCACCGGAGUCUGUCUCAUACCCAACCAGAUUUCAGUGGAGU | NO: 145 |
| 205 | | GAAGUUCAGGAGGCAUGGAGCUGACA | |
| hsa- | MIMAT00 | UGGAAUGUAAGGAAGUGUGUGG | SEQ ID |
| miR- | 00462 | | NO: 146 |
| 206 | | | |
| hsa- | MI00000 | GUAGCACUAAAGUGCUUAUAGUGCAGGUAGUGUUUAGUUA | SEQ ID |
| miR- | 76 | UCUACUGCAUUAUGAGCACUUAAAGUACUGC | NO: 147 |
| 20a | | | |
| hsa- | MI00015 | AGUACCAAAGUGCUCAUAGUGCAGGUAGUUUUGGCAUGACU | SEQ ID |
| miR- | 19 | CUACUGUAGUAUGGGCACUUCCAGUACU | NO: 148 |
| 20b | | | |
| hsa- | MI00000 | UGUCGGGUAGCUUAUCAGACUGAUGUUGACUGUUGAAUCUC | SEQ ID |
| miR-21 | 77 | AUGGCAACACCAGUCGAUGGGCUGUCUGACA | NO: 149 |
| hsa- | MI00002 | ACCCGGCAGUGCCUCCAGGCGCAGGGCAGCCCCUGCCCACCGC | SEQ ID |
| miR- | 86 | ACACUGCGCUGCCCCAGACCCACUGUGCGUGUGACAGCGGCU | NO: 150 |
| 210 | | GAUCUGUGCCUGGGCAGCGCGACCC | |
| hsa- | MI00002 | UCACCUGGCCAUGUGACUUGUGGGCUUCCCUUUGUCAUCCU | SEQ ID |
| miR- | 87 | UCGCCUAGGGCUCUGAGCAGGGCAGGGACAGCAAAGGGGUG | NO: 151 |
| 211 | | CUCAGUUGUCACUUCCCACAGCACGGAG | |
| hsa- | MI00002 | CGGGGCACCCCGCCCGGACAGCGCGCCGGCACCUUGGCUCUA | SEQ ID |
| miR- | 88 | GACUGCUUACUGCCCGGGCCGCCCUCAGUAACAGUCUCCAGU | NO: 152 |
| 212 | | CACGGCCACCGACGCCUGGCCCCGCC | |
| hsa- | I0000289 | UGAGUUUUGAGGUUGCUUCAGUGAACAUUCAACGCUGUCGG | SEQ ID |
| miR- | | UGAGUUUGGAAUUAAAAUCAAAACCAUCGACCGUUGAUUGU | NO: 153 |
| 213 | | ACCCUAUGGCUAACCAUCAUCUACUCCA | |
| hsa- | MI00002 | GGCCUGGCUGGACAGAGUUGUCAUGUGUCUGCCUGUCUACA | SEQ ID |
| miR- | 90 | CUUGCUGUGCAGAACAUCCGCUCACCUGUACAGCAGGCACAG | NO: 154 |
| 214 | | ACAGGCAGUCACAUGACAACCCAGCCU | |
| hsa- | MI00002 | AUCAUUCAGAAAUGGUAUACAGGAAAAUGACCUAUGAAUUG | SEQ ID |
| miR- | 91 | ACAGACAAUAUAGCUGAGUUUGUCUGUCAUUUCUUUAGGCC | NO: 155 |
| 215 | | AAUAUUCUGUAUGACUGUGCUACUUCAA | |
| hsa- | MI00002 | GUGAUAAUGUAGCGAGAUUUUCUGUUGUGCUUGAUCUAACC | SEQ ID |
| miR- | 94 | AUGUGGUUGCGAGGUAUGAGUAAAACAUGGUUCCGUCAAGC | NO: 156 |
| 218 | | ACCAUGGAACGUCACGCAGCUUUCUACA | |
| hsa- | MIMAT00 | UGAUUGUCCAAACGCAAUUCU | SEQ ID |
| miR- | 00276 | | NO: 157 |
| 219-5p | | | |
| hsa- | MI00000 | GGCUGAGCCGCAGUAGUUCUUCAGUGGCAAGCUUUAUGUCC | SEQ ID |
| miR-22 | 78 | UGACCCAGCUAAAGCUGCCAGUUGAAGAACUGUUGCCCUCUG | NO: 158 |
| | | CC | |
| hsa- | MI00055 | CCACACCGUAUCUGACACUUU | SEQ ID |
| miR- | 29 | | NO: 159 |
| 220 | | | |
| hsa- | MI00002 | UGAACAUCCAGGUCUGGGGCAUGAACCUGGCAUACAAUGUA | SEQ ID |
| miR- | 98 | GAUUUCUGUGUUCGUUAGGCAACAGCUACAUUGUCUGCUGG | NO: 160 |
| 221 | | GUUUCAGGCUACCUGGAAACAUGUUCUC | |
| hsa- | MI00002 | GCUGCUGGAAGGUGUAGGUACCCUCAAUGGCUCAGUAGCCA | SEQ ID |
| miR- | 99 | GUGUAGAUCCUGUCUUUCGUAAUCAGCAGCUACAUCUGGCU | NO: 161 |
| 222 | | ACUGGGUCUCUGAUGGCAUCUUCUAGCU | |
| hsa- | MI00003 | CCUGGCCUCCUGCAGUGCCACGCUCCGUGUAUUUGACAAGCU | SEQ ID |
| miR- | 00 | GAGUUGGACACUCCAUGUGGUAGAGUGUCAGUUUGUCAAAU | NO: 162 |
| 223 | | ACCCCAAGUGCGGCACAUGCUUACCAG | |
| hsa- | MI00003 | GGGCUUUCAAGUCACUAGUGGUUCCGUUUAGUAGAUGAUU | SEQ ID |
| miR- | 01 | GUGCAUUGUUUCAAAAUGGUGCCCUAGUGACUACAAAGCCC | NO: 163 |
| 224 | | | |
| hsa- | MI00000 | GGCCGGCUGGGGUUCCUGGGGAUGGGAUUUGCUUCCUGUCA | SEQ ID |
| miR- | 79 | CAAAUCACAUUGCCAGGGAUUUCCAACCGACC | NO: 164 |
| 23a | | | |
| hsa- | MI00004 | CUCAGGUGCUCUGGCUGCUUGGGUUCCUGGCAUGCUGAUUU | SEQ ID |
| miR- | 39 | GUGACUUAAGAUUAAAAUCACAUUGCCAGGGAUUACCACGCA | NO: 165 |
| 23b | | ACCACGACCUUGGC | |
| hsa- | MI00000 | CUCCGGUGCCUACUGAGCUGAUAUCAGUUCUCAUUUUACACA | SEQ ID |
| miR-24 | 80 | CUGGCUCAGUUCAGCAGGAACAGGAG | NO: 166 |
| hsa- | MI00000 | CUCUGCCUCCCGUGCCUACUGAGCUGAAACACAGUUGGUUUG | SEQ ID |
| miR- | 81 | UGUACACUGGCUCAGUUCAGCAGGAACAGGG | NO: 167 |
| 24-2 | | | |
| hsa- | MI00000 | GGCCAGUGUUGAGAGGCGGAGACUUGGGCAAUUGCUGGACG | SEQ ID |
| miR-25 | 82 | CUGCCCUGGGCAUUGCACUUGUCUCGGUCUGACAGUGCCGG | NO: 168 |
| | | CC | |
| hsa- | MI00000 | GUGGCCUCGUUCAAGUAAUCCAGGAUAGGCUGUGCAGGUCC | SEQ ID |
| miR- | 83 | CAAUGGGCCUAUUCUUGGUUACUUGCACGGGGACGC | NO: 169 |
| 26a | | | |
| hsa- | MI00007 | GGCUGUGGCUGGAUUCAAGUAAUCCAGGAUAGGCUGUUUCC | SEQ ID |
| miR- | 50 | AUCUGUGAGGCCUAUUCUUGAUUACUUGUUUCUGGAGGCAG | NO: 170 |
| 26a-2 | | CU | |
| hsa- | MI00000 | CCGGGACCCAGUUCAAGUAAUUCAGGAUAGGUUGUGUGCUG | SEQ ID |
| miR- | 84 | UCCAGCCUGUUCUCCAUUACUUGGCUCGGGGACCGG | NO: 171 |
| 26b | | | |
| hsa- | MI00000 | CUGAGGAGCAGGGCUUAGCUGCUUGUGAGCAGGGUCCACAC | SEQ ID |
| miR- | 85 | CAAGUCGUGUUCACAGUGGCUAAGUUCCGCCCCCCAG | NO: 172 |
| 27a | | | |
| hsa- | MI00004 | ACCUCUCUAACAAGGUGCAGAGCUUAGCUGAUUGGUGAACA | SEQ ID |
| miR- | 40 | GUGAUUGGUUUCCGCUUUGUUCACAGUGGCUAAGUUCUGCA | NO: 173 |
| 27b | | CCUGAAGAGAAGGUG | |
| hsa- | MIMAT00 | CACUAGAUUGUGAGCUCCUGGA | SEQ ID |
| miR- | 04502 | | NO: 174 |
| 28-3p | | | |
| hsa- | MIMAT00 | AAGGAGCUCACAGUCUAUUGAG | SEQ ID |
| miR- | 00085 | | NO: 175 |
| 28-5p | | | |
| hsa- | MIMAT00 | AGGGCCCCCCCUCAAUCCUGU | SEQ ID |
| miR- | 00690 | | NO: 176 |
| 296-5p | | | |
| hsa- | MIMAT00 | UAUGUGGGAUGGUAAACCGCUU | SEQ ID |
| miR- | 00687 | | NO: 177 |
| 299-3p | | | |
| hsa- | MIMAT00 | UGGUUUACCGUCCCACAUACAU | SEQ ID |
| miR- | 02890 | | NO: 178 |
| 299-5p | | | |
| hsa- | MI00000 | AUGACUGAUUUCUUUUGGUGUUCAGAGUCAAUAUAAUUUU | SEQ ID |
| miR- | 87 | CUAGCACCAUCUGAAAUCGGUUAU | NO: 179 |
| 29a | | | |
| hsa- | MI00001 | CUUCAGGAAGCUGGUUUCAUAUGGUGGUUUAGAUUUAAAU | SEQ ID |
| miR- | 05 | AGUGAUUGUCUAGCACCAUUUGAAAUCAGUGUUCUUGGGGG | NO: 180 |
| 29b | | | |
| hsa- | MI00001 | CUUCUGGAAGCUGGUUUCACAUGGUGGCUUAGAUUUUUCCA | SEQ ID |
| miR- | 07 | UCUUUGUAUCUAGCACCAUUUGAAAUCAGUGUUUUAGGAG | NO: 181 |
| 29b-2 | | | |
| hsa- | MI00007 | AUCUCUUACACAGGCUGACCGAUUUCUCCUGGUGUUCAGAG | SEQ ID |
| miR- | 35 | UCUGUUUUUGUCUAGCACCAUUUGAAAUCGGUUAUGAUGUA | NO: 182 |
| 29c | | GGGGGA | |
| hsa- | MI00007 | ACUGCUAACGAAUGCUCUGACUUUAUUGCACUACUGUACUU | SEQ ID |
| miR- | 45 | UACAGCUAGCAGUGCAAUAGUAUUGUCAAAGCAUCUGAAAG | NO: 183 |
| 301a | | CAGG | |
| hsa- | MIMAT00 | CAGUGCAAUGAUAUUGUCAAAGC | SEQ ID |
| miR- | 04958 | | NO: 184 |
| 301b | | | |
| hsa- | MI00007 | CCACCACUUAAACGUGGAUGUACUUGCUUUGAAACUAAAGAA | SEQ ID |
| miR- | 38 | GUAAGUGCUUCCAUGUUUUGGUGAUGG | NO: 185 |
| 302a | | | |
| hsa- | MI00007 | GCUCCCUUCAACUUUAACAUGGAAGUGCUUUCUGUGACUUU | SEQ ID |
| miR- | 72 | AAAAGUAAGUGCUUCCAUGUUUUAGUAGGAGU | NO: 186 |
| 302b | | | |
| hsa- | MI00007 | CCUUUGCUUUAACAUGGGGGUACCUGCUGUGUGAAACAAAA | SEQ ID |
| miR- | 73 | GUAAGUGCUUCCAUGUUUCAGUGGAGG | NO: 187 |
| 302c | | | |
| hsa- | MI00007 | CCUCUACUUUAACAUGGAGGCACUUGCUGUGACAUGACAAAA | SEQ ID |
| miR- | 74 | AUAAGUGCUUCCAUGUUUGAGUGUGG | NO: 188 |
| 302d | | | |
| hsa- | MIMAT00 | CUUUCAGUCGGAUGUUUGCAGC | SEQ ID |
| miR- | 00088 | | NO: 189 |
| 30a-3p | | | |
| hsa- | MIMAT00 | UGUAAACAUCCUCGACUGGAAG | SEQ ID |
| miR- | 00087 | | NO: 190 |
| 30a-5p | | | |
| hsa- | MI00004 | ACCAAGUUUCAGUUCAUGUAAACAUCCUACACUCAGCUGUAA | SEQ ID |
| miR- | 41 | UACAUGGAUUGGCUGGGAGGUGGAUGUUUACUUCAGCUGAC | NO: 191 |
| 30b | | UUGGA | |
| hsa- | MI00007 | ACCAUGCUGUAGUGUGUGUAAACAUCCUACACUCUCAGCUG | SEQ ID |
| miR- | 36 | UGAGCUCAAGGUGGCUGGGAGAGGGUUGUUUACUCCUUCU | NO: 192 |
| 30c | | GCCAUGGA | |
| hsa- | MI00002 | AGAUACUGUAAACAUCCUACACUCUCAGCUGUGGAAAGUAAG | SEQ ID |
| miR- | 54 | AAAGCUGGGAGAAGGCUGUUUACUCUUUCU | NO: 193 |
| 30c-2 | | | |
| hsa- | MI00002 | GUUGUUGUAAACAUCCCCGACUGGAAGCUGUAAGACACAGCU | SEQ ID |
| miR- | 55 | AAGCUUUCAGUCAGAUGUUUGCUGCUAC | NO: 194 |
| 30d | | | |
| hsa- | MIMAT00 | CUUUCAGUCGGAUGUUUACAGC | SEQ ID |
| miR- | 00693 | | NO: 195 |
| 30e-3p | | | |
| hsa- | MI00000 | GGAGAGGAGGCAAGAUGCUGGCAUAGCUGUUGAACUGGGAA | SEQ ID |
| miR-31 | 89 | CCUGCUAUGCCAACAUAUUGCCAUCUUUCC | NO: 196 |
| hsa- | MI00000 | GGAGAUAUUGCACAUUACUAAGUUGCAUGUUGUCACGGCCU | SEQ ID |
| miR-32 | 90 | CAAUGCAAUUUAGUGUGUGUGAUAUUUUC | NO: 197 |
| hsa- | MIMAT00 | AAAAGCUGGGUUGAGAGGGCGA | SEQ ID |
| miR- | 00510 | | NO: 198 |
| 320 | | | |
| hsa- | MIMAT00 | AAAAGCUGGGUUGAGAGGGCAA | SEQ ID |
| miR- | 05792 | | NO: 199 |
| 320b | | | |
| hsa- | MIMAT00 | CACAUUACACGGUCGACCUCU | SEQ ID |
| miR- | 00755 | | NO: 200 |
| 323a- | | | |
| 3p | | | |
| hsa- | MIMAT00 | ACUGCCCCAGGUGCUGCUGG | SEQ ID |
| miR- | 00762 | | NO: 201 |
| 324-3p | | | |
| hsa- | MIMAT00 | CGCAUCCCCUAGGGCAUUGGUGU | SEQ ID |
| miR- | 00761 | | NO: 202 |
| 324-5p | | | |
| hsa- | MIMAT00 | CCUAGUAGGUGUCCAGUAAGUGU | SEQ ID |
| miR- | 00771 | | NO: 203 |
| 325 | | | |
| hsa- | MI00008 | UGGAGUGGGGGGGCAGGAGGGGCUCAGGGAGAAAGUGCAU | SEQ ID |
| miR- | 04 | ACAGCCCCUGGCCCUCUCUGCCCUUCCGUCCCCUG | NO: 204 |
| 328 | | | |
| hsa- | MI00017 | GGUACCUGAAGAGAGGUUUUCUGGGUUUCUGUUUCUUUAA | SEQ ID |
| miR- | 25 | UGAGGACGAAACACACCUGGUUAACCUCUUUUCCAGUAUC | NO: 205 |
| 329 | | | |
| hsa- | MIMAT00 | GCAAAGCACACGGCCUGCAGAGA | SEQ ID |
| miR- | 00751 | | NO: 206 |
| 330-3p | | | |
| hsa- | MIMAT00 | GCCCCUGGGCCUAUCCUAGAA | SEQ ID |
| miR- | 00760 | | NO: 207 |
| 331-3p | | | |
| hsa- | MIMAT00 | CUAGGUAUGGUCCCAGGGAUCC | SEQ ID |
| miR- | 04700 | | NO: 208 |
| 331-5p | | | |
| hsa- | MI00008 | UGUUUUGAGCGGGGGUCAAGAGCAAUAACGAAAAAUGUUUG | SEQ ID |
| miR- | 16 | UCAUAAACCGUUUUUCAUUAUUGCUCCUGACCUCCUCUCAUU | NO: 209 |
| 335 | | UGCUAUAUUCA | |
| hsa- | MIMAT00 | GAACGGCUUCAUACAGGAGUU | SEQ ID |
| miR- | 04695 | | NO: 210 |
| 337-5p | | | |
| hsa- | MIMAT00 | UCCAGCAUCAGUGAUUUUGUUG | SEQ ID |
| miR- | 00763 | | NO: 211 |
| 338-3p | | | |
| hsa- | MIMAT00 | AACAAUAUCCUGGUGCUGAGUG | SEQ ID |
| miR- | 04701 | | NO: 212 |
| 338-5p | | | |
| hsa- | MIMAT00 | UGAGCGCCUCGACGACAGAGCCG | SEQ ID |
| miR- | 04702 | | NO: 213 |
| 339-3p | | | |
| hsa- | MIMAT00 | UCCCUGUCCUCCAGGAGCUCACG | SEQ ID |
| miR- | 00764 | | NO: 214 |
| 339-5p | | | |
| hsa- | MI00000 | CUGUGGUGCAUUGUAGUUGCAUUGCAUGUUCUGGUGGUAC | SEQ ID |
| miR- | 91 | CCAUGCAAUGUUUCCACAGUGCAUCACAG | NO: 215 |
| 33a | | | |
| hsa- | MI00036 | GCGGGCGGCCCCGCGGUGCAUUGCUGUUGCAUUGCACGUGU | SEQ ID |
| miR- | 46 | GUGAGGCGGGUGCAGUGCCUCGGCAGUGCAGCCCGGAGCCG | NO: 216 |
| 33b | | GCCCCUGGCACCAC | |
| hsa- | MI00008 | UUGUACCUGGUGUGAUUAUAAAGCAAUGAGACUGAUUGUCA | SEQ ID |
| miR- | 02 | UAUGUCGUUUGUGGGAUCCGUCUCAGUUACUUUAUAGCCAU | NO: 217 |
| 340 | | ACCUGGUAUCUUA | |
| hsa- | MIMAT00 | UCUCACACAGAAAUCGCACCCGU | SEQ ID |
| miR- | 00753 | | NO: 218 |
| 342-3p | | | |
| hsa- | MIMAT00 | AGGGGUGCUAUCUGUGAUUGA | SEQ ID |
| miR- | 04694 | | NO: 219 |
| 342-5p | | | |
| hsa- | MI00008 | ACCCAAACCCUAGGUCUGCUGACUCCUAGUCCAGGGCUCGUG | SEQ ID |
| miR- | 25 | AUGGCUGGUGGGCCCUGAACGAGGGGUCUGGAGGCCUGGGU | NO: 220 |
| 345 | | UUGAAUAUCGACAGC | |
| hsa- | MIMAT00 | UGUCUGCCCGCAUGCCUGCCUCU | SEQ ID |
| miR- | 00773 | | NO: 221 |
| 346 | | | |
| hsa- | MI00002 | GGCCAGCUGUGAGUGUUUCUUUGGCAGUGUCUUAGCUGGU | SEQ ID |
| miR- | 68 | UGUUGUGAGCAAUAGUAAGGAAGCAAUCAGCAAGUAUACUG | NO: 222 |
| 34a | | CCCUAGAAGUGCUGCACGUUGUGGGGCCC | |
| hsa- | MI00007 | GUGCUCGGUUUGUAGGCAGUGUCAUUAGCUGAUUGUACUG | SEQ ID |
| miR- | 42 | UGGUGGUUACAAUCACUAACUCCACUGCCAUCAAAACAAGGC | NO: 223 |
| 34b | | AC | |
| hsa- | MIMAT00 | UCCCCCAGGUGUGAUUCUGAUUU | SEQ ID |
| miR- | 04682 | | NO: 224 |
| 361-3p | | | |
| hsa- | MIMAT00 | UUAUCAGAAUCUCCAGGGGUAC | SEQ ID |
| miR- | 00703 | | NO: 225 |
| 361-5p | | | |
| hsa- | MIMAT00 | AAUCCUUGGAACCUAGGUGUGAGU | SEQ ID |
| miR- | 00705 | | NO: 226 |
| 362-5p | | | |
| hsa- | MI00007 | UGUUGUCGGGUGGAUCACGAUGCAAUUUUGAUGAGUAUCA | SEQ ID |
| miR- | 64 | UAGGAGAAAAAUUGCACGGUAUCCAUCUGUAAACC | NO: 227 |
| 363 | | | |
| hsa- | MI00007 | ACCGCAGGGAAAAUGAGGGACUUUUGGGGGCAGAUGUGUUU | SEQ ID |
| miR- | 67 | CCAUUCCACUAUCAUAAUGCCCCUAAAAAUCCUUAUUGCUCU | NO: 228 |
| 365 | | UGCA | |
| hsa- | MI00007 | CCAUUACUGUUGCUAAUAUGCAACUCUGUUGAAUAUAAAUU | SEQ ID |
| miR- | 75 | GGAAUUGCACUUUAGCAAUGGUGAUGG | NO: 229 |
| 367 | | | |
| hsa- | MIMAT00 | AAUAAUACAUGGUUGAUCUUU | SEQ ID |
| miR- | 00721 | | NO: 230 |
| 369-3p | | | |
| hsa- | MI00007 | AGACAGAGAAGCCAGGUCACGUCUCUGCAGUUACACAGCUCA | SEQ ID |
| miR- | 78 | CGAGUGCCUGCUGGGGUGGAACCUGGUCUGUCU | NO: 231 |
| 370 | | | |
| hsa- | MI00007 | GUGGGCCUCAAAUGUGGAGCACUAUUCUGAUGUCCAAGUGG | SEQ ID |
| miR- | 80 | AAAGUGCUGCGACAUUUGAGCGUCAC | NO: 232 |
| 372 | | | |
| hsa- | MI00007 | GGGAUACUCAAAAUGGGGGCGCUUUCCUUUUUGUCUGUACU | SEQ ID |
| miR- | 81 | GGGAAGUGCUUCGAUUUUGGGGUGUCCC | NO: 233 |
| 373 | | | |
| hsa- | MI00007 | UACAUCGGCCAUUAUAAUACAACCUGAUAAGUGUUAUAGCAC | SEQ ID |
| miR- | 82 | UUAUCAGAUUGUAUUGUAAUUGUCUGUGUA | NO: 234 |
| 374a | | | |
| hsa- | MI00055 | ACUCGGAUGGAUAUAAUACAACCUGCUAAGUGUCCUAGCACU | SEQ ID |
| miR- | 66 | UAGCAGGUUGUAUUAUCAUUGUCCGUGUCU | NO: 235 |
| 374b | | | |
| hsa- | MIMAT00 | UUUGUUCGUUCGGCUCGCGUGA | SEQ ID |
| miR- | 00728 | | NO: 236 |
| 375 | | | |
| hsa- | MI00007 | UAAAAGGUAGAUUCUCCUUCUAUGAGUACAUUAUUUAUGAU | SEQ ID |
| miR- | 84 | UAAUCAUAGAGGAAAAUCCACGUUUUC | NO: 237 |
| 376a | | | |
| hsa- | MI00024 | CAGUCCUUCUUUGGUAUUUAAAACGUGGAUAUUCCUUCUAU | SEQ ID |
| miR- | 66 | GUUUACGUGAUUCCUGGUUAAUCAUAGAGGAAAAUCCAUGU | NO: 238 |
| 376b | | UUUCAGUAUCAAAUGCUG | |
| hsa- | MI00007 | AAAAGGUGGAUAUUCCUUCUAUGUUUAUGUUAUUUAUGGU | SEQ ID |
| miR- | 76 | UAAACAUAGAGGAAAUUCCACGUUUU | NO: 239 |
| 376c | | | |
| hsa- | MI00007 | AGGGCUCCUGACUCCAGGUCCUGUGUGUUACCUAGAAAUAG | SEQ ID |
| miR- | 86 | CACUGGACUUGGAGUCAGAAGGCCU | NO: 240 |
| 378 | | | |
| hsa- | MI00007 | AGAGAUGGUAGACUAUGGAACGUAGGCGUUAUGAUUUCUGA | SEQ ID |
| miR- | 87 | CCUAUGUAACAUGGUCCACUAACUCU | NO: 241 |
| 379 | | | |
| hsa- | MI00007 | AAGAUGGUUGACCAUAGAACAUGCGCUAUCUCUGUGUCGUA | SEQ ID |
| miR- | 88 | UGUAAUAUGGUCCACAUCUU | NO: 242 |
| 380 | | | |
| hsa- | MI00007 | UACUUAAAGCGAGGUUGCCCUUUGUAUAUUCGGUUUAUUGA | SEQ ID |
| miR- | 89 | CAUGGAAUAUACAAGGGCAAGCUCUCUGUGAGUA | NO: 243 |
| 381 | | | |
| hsa- | MI00007 | UACUUGAAGAGAAGUUGUUCGUGGUGGAUUCGCUUUACUU | SEQ ID |
| miR- | 90 | AUGACGAAUCAUUCACGGACAACACUUUUUUCAGUA | NO: 244 |
| 382 | | | |
| hsa- | MIMAT00 | AUUCCUAGAAAUUGUUCAUA | SEQ ID |
| miR- | 01075 | | NO: 245 |
| 384 | | | |
| hsa- | MIMAT00 | GAAUGUUGCUCGGUGAACCCCU | SEQ ID |
| miR- | 01639 | | NO: 246 |
| 409-3p | | | |
| hsa- | MIMAT00 | AGGUUACCCGAGCAACUUUGCAU | SEQ ID |
| miR- | 01638 | | NO: 247 |
| 409-5p | | | |
| hsa- | MI00024 | GGUACCUGAGAAGAGGUUGUCUGUGAUGAGUUCGCUUUUA | SEQ ID |
| miR- | 65 | UUAAUGACGAAUAUAACACAGAUGGCCUGUUUUCAGUACC | NO: 248 |
| 410 | | | |
| hsa- | MI00036 | UGGUACUUGGAGAGAUAGUAGACCGUAUAGCGUACGCUUUA | SEQ ID |
| miR- | 75 | UCUGUGACGUAUGUAACACGGUCCACUAACCCUCAGUAUCAA | NO: 249 |
| 411 | | AUCCAUCCCCGAG | |
| hsa- | MIMAT00 | AUCAACAGACAUUAAUUGGGCGC | SEQ ID |
| miR- | 03339 | | NO: 250 |
| 421 | | | |
| hsa- | MIMAT00 | ACUGGACUUAGGGUCAGAAGGC | SEQ ID |
| miR- | 01339 | | NO: 251 |
| 422a | | | |
| hsa- | MIMAT00 | AGCUCGGUCUGAGGCCCCUCAGU | SEQ ID |
| miR- | 01340 | | NO: 252 |
| 423-3p | | | |
| hsa- | MIMAT00 | UGAGGGGCAGAGAGCGAGACUUU | SEQ ID |
| miR- | 04748 | | NO: 253 |
| 423-5p | | | |
| hsa- | MI00014 | CGAGGGGAUACAGCAGCAAUUCAUGUUUUGAAGUGUUCUAA | SEQ ID |
| miR- | 46 | AUGGUUCAAAACGUGAGGCGCUGCUAUACCCCCUCGUGGGG | NO: 254 |
| 424 | | AAGGUAGAAGGUGGGG | |
| hsa- | MI00014 | GAAAGCGCUUUGGAAUGACACGAUCACUCCCGUUGAGUGGG | SEQ ID |
| miR- | 48 | CACCCGAGAAGCCAUCGGGAAUGUCGUGUCCGCCCAGUGCUC | NO: 255 |
| 425 | | UUUC | |
| hsa- | MIMAT00 | UAAUACUGUCUGGUAAAACCGU | SEQ ID |
| miR- | 01536 | | NO: 256 |
| 429 | | | |
| hsa- | MI00017 | UCCUGCUUGUCCUGCGAGGUGUCUUGCAGGCCGUCAUGCAG | SEQ ID |
| miR- | 21 | GCCACACUGACGGUAACGUUGCAGGUCGUCUUGCAGGGCUU | NO: 257 |
| 431 | | CUCGCAAGACGACAUCCUCAUCACCAACGACG | |
| hsa- | MI00031 | UGACUCCUCCAGGUCUUGGAGUAGGUCAUUGGGUGGAUCCU | SEQ ID |
| miR- | 33 | CUAUUUCCUUACGUGGGCCACUGGAUGGCUCCUCCAUGUCU | NO: 258 |
| 432 | | UGGAGUAGAUCA | |
| hsa- | MI00017 | CCGGGGAGAAGUACGGUGAGCCUGUCAUUAUUCAGAGAGGC | SEQ ID |
| miR- | 23 | UAGAUCCUCUGUGUUGAGAAGGAUCAUGAUGGGCUCCUCGG | NO: 259 |
| 433 | | UGUUCUCCAGG | |
| hsa- | MIMAT00 | UGGCAGUGUAUUGUUAGCUGGU | SEQ ID |
| miR- | 01541 | | NO: 260 |
| 449a | | | |
| hsa- | MI00016 | AAACGAUACUAAACUGUUUUUGCGAUGUGUUCCUAAUAUGC | SEQ ID |
| miR- | 52 | ACUAUAAAUAUAUUGGGAACAUUUUGCAUGUAUAGUUUUG | NO: 261 |
| 450a | | UAUCAAUAUA | |
| hsa- | MIMAT00 | UUGGGAUCAUUUUGCAUCCAUA | SEQ ID |
| miR- | 04910 | | NO: 262 |
| 450b- | | | |
| 3p | | | |
| hsa- | MIMAT00 | UUUUGCAAUAUGUUCCUGAAUA | SEQ ID |
| miR- | 04909 | | NO: 263 |
| 450b- | | | |
| 5p | | | |
| hsa- | MIMAT00 | AAACCGUUACCAUUACUGAGUU | SEQ ID |
| miR- | 01631 | | NO: 264 |
| 451 | | | |
| hsa- | MI00017 | GCUAAGCACUUACAACUGUUUGCAGAGGAAACUGAGACUUU | SEQ ID |
| miR- | 33 | GUAACUAUGUCUCAGUCUCAUCUGCAAAGAAGUAAGUGCUU | NO: 265 |
| 452 | | UGC | |
| hsa- | MI00038 | UCUGUUUAUCACCAGAUCCUAGAACCCUAUCAAUAUUGUCUC | SEQ ID |
| miR- | 20 | UGCUGUGUAAAUAGUUCUGAGUAGUGCAAUAUUGCUUAUA | NO: 266 |
| 454 | | GGGUUUUGGUGUUUGGAAAGAACAAUGGGCAGG | |
| hsa- | MIMAT00 | GCAGUCCAUGGGCAUAUACAC | SEQ ID |
| miR- | 04784 | | NO: 267 |
| 455-3p | | | |
| hsa- | MIMAT00 | UCACUCCUCUCCUCCCGUCUU | SEQ ID |
| miR- | 02173 | | NO: 268 |
| 483-3p | | | |
| hsa- | MIMAT00 | AAGACGGGAGGAAAGAAGGGAG | SEQ ID |
| miR- | 04761 | | NO: 269 |
| 483-5p | | | |
| hsa- | MIMAT00 | UCAGGCUCAGUCCCCUCCCGAU | SEQ ID |
| miR- | 02174 | | NO: 270 |
| 484 | | | |
| hsa- | MIMAT00 | GUCAUACACGGCUCUCCUCUCU | SEQ ID |
| miR- | 02176 | | NO: 271 |
| 485-3p | | | |
| hsa- | MIMAT00 | AGAGGCUGGCCGUGAUGAAUUC | SEQ ID |
| miR- | 02175 | | NO: 272 |
| 485-5p | | | |
| hsa- | MIMAT00 | CGGGGCAGCUCAGUACAGGAU | SEQ ID |
| miR- | 04762 | | NO: 273 |
| 486-3p | | | |
| hsa- | MIMAT00 | UCCUGUACUGAGCUGCCCCGAG | SEQ ID |
| miR- | 02177 | | NO: 274 |
| 486-5p | | | |
| hsa- | MI00024 | GGUACUUGAAGAGUGGUUAUCCCUGCUGUGUUCGCUUAAUU | SEQ ID |
| miR- | 71 | UAUGACGAAUCAUACAGGGACAUCCAGUUUUUCAGUAUC | NO: 275 |
| 487a | | | |
| hsa- | MI00035 | UUGGUACUUGGAGAGUGGUUAUCCCUGUCCUGUUCGUUUU | SEQ ID |
| miR- | 30 | GCUCAUGUCGAAUCGUACAGGGUCAUCCACUUUUUCAGUAU | NO: 276 |
| 487b | | CAA | |
| hsa- | MI00031 | GAGAAUCAUCUCUCCCAGAUAAUGGCACUCUCAAACAAGUUU | SEQ ID |
| miR- | 23 | CCAAAUUGUUUGAAAGGCUAUUUCUUGGUCAGAUGACUCUC | NO: 277 |
| 488 | | | |
| hsa- | MI00031 | GUGGCAGCUUGGUGGUCGUAUGUGUGACGCCAUUUACUUG | SEQ ID |
| miR- | 24 | AACCUUUAGGAGUGACAUCACAUAUACGGCAGCUAAACUGCU | NO: 278 |
| 489 | | AC | |
| hsa- | MIMAT00 | CAACCUGGAGGACUCCAUGCUG | SEQ ID |
| miR- | 02806 | | NO: 279 |
| 490-3p | | | |
| hsa- | MIMAT00 | AGUGGGGAACCCUUCCAUGAGG | SEQ ID |
| miR- | 02807 | | NO: 280 |
| 491-5p | | | |
| hsa- | MIMAT00 | AGGACCUGCGGGACAAGAUUCUU | SEQ ID |
| miR- | 02812 | | NO: 281 |
| 492 | | | |
| hsa- | MI00031 | CUGGCCUCCAGGGCUUUGUACAUGGUAGGCUUUCAUUCAUU | SEQ ID |
| miR- | 32 | CGUUUGCACAUUCGGUGAAGGUCUACUGUGUGCCAGGCCCU | NO: 282 |
| 493 | | GUGCCAG | |
| hsa- | MIMAT00 | UGAAGGUCUACUGUGUGCCAGG | SEQ ID |
| miR- | 03161 | | NO: 283 |
| 493-3p | | | |
| hsa- | MI00031 | GAUACUCGAAGGAGAGGUUGUCCGUGUUGUCUUCUCUUUAU | SEQ ID |
| miR- | 34 | UUAUGAUGAAACAUACACGGGAAACCUCUUUUUUAGUAUC | NO: 284 |
| 494 | | | |
| hsa- | MI00031 | UGGUACCUGAAAAGAAGUUGCCCAUGUUAUUUUCGCUUUAU | SEQ ID |
| miR- | 35 | AUGUGACGAAACAAACAUGGUGCACUUCUUUUUCGGUAUCA | NO: 285 |
| 495 | | | |
| hsa- | MI00031 | CCACCCCGGUCCUGCUCCCGCCCCAGCAGCACACUGUGGUUUG | SEQ ID |
| miR- | 38 | UACGGCACUGUGGCCACGUCCAAACCACACUGUGGUGUUAGA | NO: 286 |
| 497 | | GCGAGGGUGGGGGAGGCACCGCCGAGG | |
| hsa- | MIMAT00 | AACAUCACAGCAAGUCUGUGCU | SEQ ID |
| miR- | 04772 | | NO: 287 |
| 499a- | | | |
| 3p | | | |
| hsa- | MIMAT00 | UUAAGACUUGCAGUGAUGUUU | SEQ ID |
| miR- | 02870 | | NO: 288 |
| 499a- | | | |
| 5p | | | |
| hsa- | MI00031 | GCUCCCCCUCUCUAAUCCUUGCUACCUGGGUGAGAGUGCUG | SEQ ID |
| miR- | 84 | UCUGAAUGCAAUGCACCUGGGCAAGGAUUCUGAGAGCGAGA | NO: 289 |
| 500 | | GC | |
| hsa- | MIMAT00 | AAUCCUUUGUCCCUGGGUGAGA | SEQ ID |
| miR- | 02872 | | NO: 290 |
| 501-5p | | | |
| hsa- | MIMAT00 | AAUGCACCUGGGCAAGGAUUCA | SEQ ID |
| miR- | 04775 | | NO: 291 |
| 502-3p | | | |
| hsa- | MIMAT00 | AUCCUUGCUAUCUGGGUGCUA | SEQ ID |
| miR- | 02873 | | NO: 292 |
| 502-5p | | | |
| hsa- | MI00031 | GAUGCACCCAGUGGGGGAGCCAGGAAGUAUUGAUGUUUCUG | SEQ ID |
| miR- | 90 | CCAGUUUAGCGUCAACACUUGCUGGUUUCCUCUCUGGAGCA | NO: 293 |
| 505 | | UC | |
| hsa- | MI00031 | GCCACCACCAUCAGCCAUACUAUGUGUAGUGCCUUAUUCAGG | SEQ ID |
| miR- | 93 | AAGGUGUUACUUAAUAGAUUAAUAUUUGUAAGGCACCCUUC | NO: 294 |
| 506 | | UGAGUAGAGUAAUGUGCAACAUGGACAACAUUUGUGGUGGC | |
| hsa- | MIMAT00 | UUUUGCACCUUUUGGAGUGAA | SEQ ID |
| miR- | 02879 | | NO: 295 |
| 507 | | | |
| hsa- | MIMAT00 | UGAUUGUAGCCUUUUGGAGUAGA | SEQ ID |
| miR- | 02880 | | NO: 296 |
| 508-3p | | | |
| hsa- | MIMAT00 | UACUGCAGACAGUGGCAAUCA | SEQ ID |
| miR- | 04779 | | NO: 297 |
| 509-5p | | | |
| hsa- | MI00031 | CAAUAGACACCCAUCGUGUCUUUUGCUCUGCAGUCAGUAAAU | SEQ ID |
| miR- | 27 | AUUUUUUUGUGAAUGUGUAGCAAAAGACAGAAUGGUGGUCC | NO: 298 |
| 511 | | AUUG | |
| hsa- | MIMAT00 | AAGUGCUGUCAUAGCUGAGGUC | SEQ ID |
| miR- | 02823 | | NO: 299 |
| 512-3p | | | |
| hsa- | MI00066 | GCGUACAGUGCCUUUCUCAAGGAGGUGUCGUUUAUGUGAAC | SEQ ID |
| miR- | 49 | UAAAAUAUAAAUUUCACCUUUCUGAGAAGAGUAAUGUACAG | NO: 300 |
| 513c | | CA | |
| hsa- | MIMAT00 | GAGUGCCUUCUUUUGGAGCGUU | SEQ ID |
| miR- | 02827 | | NO: 301 |
| 515-3p | | | |
| hsa- | MIMAT00 | UGCUUCCUUUCAGAGGGU | SEQ ID |
| miR- | 06778 | | NO: 302 |
| 516a- | | | |
| 3p | | | |
| hsa- | MI00031 | UCUCAGGCAGUGACCCUCUAGAUGGAAGCACUGUCUGUUGU | SEQ ID |
| miR- | 61 | AUAAAAGAAAAGAUCGUGCAUCCCUUUAGAGUGUUACUGUU | NO: 303 |
| 517 | | UGAGA | |
| hsa- | MI00031 | GUGACCCUCUAGAUGGAAGCACUGUCUGUUGUCUAAGAAAA | SEQ ID |
| miR- | 65 | GAUCGUGCAUCCCUUUAGAGUGUUAC | NO: 304 |
| 517b | | | |
| hsa- | MI00031 | GAAGAUCUCAGGCAGUGACCCUCUAGAUGGAAGCACUGUCU | SEQ ID |
| miR- | 74 | GUUGUCUAAGAAAAGAUCGUGCAUCCUUUUAGAGUGUUACU | NO: 305 |
| 517c | | GUUUGAGAAAAUC | |
| hsa- | MIMAT00 | CAAAGCGCUCCCCUUUAGAGGU | SEQ ID |
| miR- | 02844 | | NO: 306 |
| 518b | | | |
| hsa- | MIMAT00 | CAAAGCGCUUCCCUUUGGAGC | SEQ ID |
| miR- | 02864 | | NO: 307 |
| 518d- | | | |
| 3p | | | |
| hsa- | MIMAT00 | CUCUAGAGGGAAGCACUUUCUG | SEQ ID |
| miR- | 05456 | | NO: 308 |
| 518d- | | | |
| 5p | | | |
| hsa- | MI00031 | UCUCAGGCUGUGACCCUCUAGAGGGAAGCGCUUUCUGUUGG | SEQ ID |
| miR- | 69 | CUAAAAGAAAAGAAAGCGCUUCCCUUCAGAGUGUUAACGCUU | NO: 309 |
| 518e | | UGAGA | |
| hsa- | MI00031 | UCUCAUGCUGUGACCCUCUAGAGGGAAGCACUUUCUCUUGU | SEQ ID |
| miR- | 54 | CUAAAAGAAAAGAAAGCGCUUCUCUUUAGAGGAUUACUCUU | NO: 310 |
| 518f | | UGAGA | |
| hsa- | MI00031 | CUCAGGCUGUGACACUCUAGAGGGAAGCGCUUUCUGUUGUC | SEQ ID |
| miR- | 78 | UGAAAGAAAGGAAAGUGCAUCCUUUUAGAGUGUUACUGUUU | NO: 311 |
| 519a | | GAG | |
| hsa- | MI00031 | UCCCAUGCUGUGACCCUCCAAAGGGAAGCGCUUUCUGUUUG | SEQ ID |
| miR- | 62 | UUUUCUCUUAAACAAAGUGCCUCCCUUUAGAGUGUUACCGU | NO: 312 |
| 519d | | UUGGGA | |
| hsa- | MI00031 | UCUCAUGCAGUCAUUCUCCAAAAGGGAGCACUUUCUGUUUG | SEQ ID |
| miR- | 45 | AAAGAAAACAAAGUGCCUCCUUUUAGAGUGUUACUGUUUGA | NO: 313 |
| 519e | | GA | |
| hsa- | MIMAT00 | CUCCAGAGGGAAGUACUUUCU | SEQ ID |
| miR- | 02833 | | NO: 314 |
| 520a- | | | |
| 5p | | | |
| hsa- | MIMAT00 | AAAGUGCUUCCUUUUAGAGGG | SEQ ID |
| miR- | 02843 | | NO: 315 |
| 520b | | | |
| hsa- | MIMAT00 | AAAGUGCUUCCUUUUAGAGGGU | SEQ ID |
| miR- | 02846 | | NO: 316 |
| 520c- | | | |
| 3p | | | |
| hsa- | MIMAT00 | AAAGUGCUUCUCUUUGGUGGGU | SEQ ID |
| miR- | 02856 | | NO: 317 |
| 520d- | | | |
| 3p | | | |
| hsa- | MIMAT00 | CUACAAAGGGAAGCCCUUUC | SEQ ID |
| miR- | 02855 | | NO: 318 |
| 520d- | | | |
| 5p | | | |
| hsa- | MI00031 | UCCCAUGCUGUGACCCUCUAGAGGAAGCACUUUCUGUUUGU | SEQ ID |
| miR- | 66 | UGUCUGAGAAAAAACAAAGUGCUUCCCUUUAGAGUGUUACC | NO: 319 |
| 520g | | GUUUGGGA | |
| hsa- | MIMAT00 | AACGCACUUCCCUUUAGAGUGU | SEQ ID |
| miR- | 02854 | | NO: 320 |
| 521 | | | |
| hsa- | MI00031 | UCUCAGGCUGUGUCCCUCUAGAGGGAAGCGCUUUCUGUUGU | SEQ ID |
| miR- | 77 | CUGAAAGAAAAGAAAAUGGUUCCCUUUAGAGUGUUACGCUU | NO: 321 |
| 522 | | UGAGA | |
| hsa- | MI00031 | UCUCAUGCUGUGACCCUCUAGAGGGAAGCGCUUUCUGUUGU | SEQ ID |
| miR- | 53 | CUGAAAGAAAAGAACGCGCUUCCCUAUAGAGGGUUACCCUUU | NO: 322 |
| 523 | | GAGA | |
| hsa- | MIMAT00 | GAAGGCGCUUCCCUUUAGAGCG | SEQ ID |
| miR- | 02839 | | NO: 323 |
| 525-3p | | | |
| hsa- | MIMAT00 | CCUCCCACACCCAAGGCUUGCA | SEQ ID |
| miR- | 04780 | | NO: 324 |
| 532-3p | | | |
| hsa- | MIMAT00 | CAUGCCUUGAGUGUAGGACCGU | SEQ ID |
| miR- | 02888 | | NO: 325 |
| 532-5p | | | |
| hsa- | MI00035 | AUACUUGAGGAGAAAUUAUCCUUGGUGUGUUCGCUUUAUU | SEQ ID |
| miR- | 14 | UAUGAUGAAUCAUACAAGGACAAUUUCUUUUUGAGUAU | NO: 326 |
| 539 | | | |
| hsa- | MI00055 | ACGUCAGGGAAAGGAUUCUGCUGUCGGUCCCACUCCAAAGU | SEQ ID |
| miR- | 39 | UCACAGAAUGGGUGGUGGGCACAGAAUCUGGACUCUGCUUG | NO: 327 |
| 541 | | UG | |
| hsa- | MIMAT00 | AAACAUUCGCGGUGCACUUCUU | SEQ ID |
| miR- | 04954 | | NO: 328 |
| 543 | | | |
| hsa- | MI00035 | CCCAGCCUGGCACAUUAGUAGGCCUCAGUAAAUGUUUAUUA | SEQ ID |
| miR- | 16 | GAUGAAUAAAUGAAUGACUCAUCAGCAAACAUUUAUUGUGU | NO: 329 |
| 545 | | GCCUGCUAAAGUGAGCUCCACAGG | |
| hsa- | MIMAT00 | CAAAACUGGCAAUUACUUUUGC | SEQ ID |
| miR- | 03251 | | NO: 330 |
| 548a- | | | |
| 3p | | | |
| hsa- | MIMAT00 | AAAAGUAAUUGCGAGUUUUACC | SEQ ID |
| miR- | 04803 | | NO: 331 |
| 548a- | | | |
| 5p | | | |
| hsa- | MIMAT00 | AAAAGUAAUUGUGGUUUUGGCC | SEQ ID |
| miR- | 04798 | | NO: 332 |
| 548b- | | | |
| 5p | | | |
| hsa- | MIMAT00 | CAAAAAUCUCAAUUACUUUUGC | SEQ ID |
| miR- | 03285 | | NO: 333 |
| 548c- | | | |
| 3p | | | |
| hsa- | MI00064 | UCUGUCCAUUAGGUGGGUGCAAAAGUAAUCGCGGUUUUUG | SEQ ID |
| miR- | 11 | UCAUUACUUUUAAUGGUAAAAACUGGAAUUACUUUUGCACU | NO: 334 |
| 548h | | GACCUAAUAUUAAGCCAGAUA | |
| hsa- | MIMAT00 | CAAAAGUAAUUGUGGAUUUUGU | SEQ ID |
| miR- | 05916 | | NO: 335 |
| 548n | | | |
| hsa- | MI00035 | AGAUGUGCUCUCCUGGCCCAUGAAAUCAAGCGUGGGUGAGA | SEQ ID |
| miR- | 75 | CCUGGUGCAGAACGGGAAGGCGACCCAUACUUGGUUUCAGA | NO: 336 |
| 551b | | GGCUGUGAGAAUAA | |
| hsa- | MI00035 | AACCAUUCAAAUAUACCACAGUUUGUUUAACCUUUUGCCUG | SEQ ID |
| miR- | 57 | UUGGUUGAAGAUGCCUUUCAACAGGUGACUGGUUAGACAAA | NO: 337 |
| 552 | | CUGUGGUAUAUACA | |
| hsa- | MIMAT00 | GCUAGUCCUGACUCAGCCAGU | SEQ ID |
| miR- | 03217 | | NO: 338 |
| 554 | | | |
| hsa- | MIMAT00 | GAUGAGCUCAUUGUAAUAUGAG | SEQ ID |
| miR- | 03220 | | NO: 339 |
| 556-5p | | | |
| hsa- | MIMAT00 | GUUUGCACGGGUGGGCCUUGUCU | SEQ ID |
| miR- | 03221 | | NO: 340 |
| 557 | | | |
| hsa- | MIMAT00 | AAAGUAGCUGUACCAUUUGC | SEQ ID |
| miR- | 03226 | | NO: 341 |
| 562 | | | |
| hsa- | MIMAT00 | AGGCACGGUGUCAGCAGGC | SEQ ID |
| miR- | 03228 | | NO: 342 |
| 564 | | | |
| hsa- | MIMAT00 | GGGCGCCUGUGAUCCCAAC | SEQ ID |
| miR- | 03230 | | NO: 343 |
| 566 | | | |
| hsa- | MIMAT00 | AGUUAAUGAAUCCUGGAAAGU | SEQ ID |
| miR- | 03234 | | NO: 344 |
| 569 | | | |
| hsa- | MI00035 | CUAGAUAAGUUAUUAGGUGGGUGCAAAGGUAAUUGCAGUU | SEQ ID |
| miR- | 77 | UUUCCCAUUAUUUUAAUUGCGAAAACAGCAAUUACCUUUGC | NO: 345 |
| 570 | | ACCAACCUGAUGGAGU | |
| hsa- | MIMAT00 | UGAGUUGGCCAUCUGAGUGAG | SEQ ID |
| miR- | 03236 | | NO: 346 |
| 571 | | | |
| hsa- | MIMAT00 | GUCCGCUCGGCGGUGGCCCA | SEQ ID |
| miR- | 03237 | | NO: 347 |
| 572 | | | |
| hsa- | MIMAT00 | CACGCUCAUGCACACACCCACA | SEQ ID |
| miR- | 03239 | | NO: 348 |
| 574-3p | | | |
| hsa- | MIMAT00 | AAGAUGUGGAAAAAUUGGAAUC | SEQ ID |
| miR- | 04796 | | NO: 349 |
| 576-3p | | | |
| hsa- | MIMAT00 | AUUCUAAUUUCUCCACGUCUUU | SEQ ID |
| miR- | 03241 | | NO: 350 |
| 576-5p | | | |
| hsa- | MIMAT00 | UAGAUAAAAUAUUGGUACCUG | SEQ ID |
| miR- | 03242 | | NO: 351 |
| 577 | | | |
| hsa- | MIMAT00 | CUUCUUGUGCUCUAGGAUUGU | SEQ ID |
| miR- | 03243 | | NO: 352 |
| 578 | | | |
| hsa- | MI00035 | CAUAUUAGGUUAAUGCAAAAGUAAUCGCGGUUUGUGCCAGA | SEQ ID |
| miR- | 86 | UGACGAUUUGAAUUAAUAAAUUCAUUUGGUAUAAACCGCGA | NO: 353 |
| 579 | | UUAUUUUUGCAUCAAC | |
| hsa- | MIMAT00 | UCUUGUGUUCUCUAGAUCAGU | SEQ ID |
| miR- | 03246 | | NO: 354 |
| 581 | | | |
| hsa- | MIMAT00 | UAACUGGUUGAACAACUGAACC | SEQ ID |
| miR- | 04797 | | NO: 355 |
| 582-3p | | | |
| hsa- | MIMAT00 | UUACAGUUGUUCAACCAGUUACU | SEQ ID |
| miR- | 03247 | | NO: 356 |
| 582-5p | | | |
| hsa- | MIMAT00 | CAAAGAGGAAGGUCCCAUUAC | SEQ ID |
| miR- | 03248 | | NO: 357 |
| 583 | | | |
| hsa- | MI00035 | UAGGGUGACCAGCCAUUAUGGUUUGCCUGGGACUGAGGAAU | SEQ ID |
| miR- | 91 | UUGCUGGGAUAUGUCAGUUCCAGGCCAACCAGGCUGGUUGG | NO: 358 |
| 584 | | UCUCCCUGAAGCAAC | |
| hsa- | MIMAT00 | UUUCCAUAGGUGAUGAGUCAC | SEQ ID |
| miR- | 03253 | | NO: 359 |
| 587 | | | |
| hsa- | MIMAT00 | UUGGCCACAAUGGGUUAGAAC | SEQ ID |
| miR- | 03255 | | NO: 360 |
| 588 | | | |
| hsa- | MI00035 | UCCAGCCUGUGCCCAGCAGCCCCUGAGAACCACGUCUGCUCU | SEQ ID |
| miR- | 99 | GAGCUGGGUACUGCCUGUUCAGAACAAAUGCCGGUUCCCAGA | NO: 361 |
| 589 | | CGCUGCCAGCUGGCC | |
| hsa- | MIMAT00 | UAAUUUUAUGUAUAAGCUAGU | SEQ ID |
| miR- | 04801 | | NO: 362 |
| 590-3p | | | |
| hsa- | MIMAT00 | GAGCUUAUUCAUAAAAGUGCAG | SEQ ID |
| miR- | 03258 | | NO: 363 |
| 590-5p | | | |
| hsa- | MIMAT00 | AGACCAUGGGUUCUCAUUGU | SEQ ID |
| miR- | 03259 | | NO: 364 |
| 591 | | | |
| hsa- | MIMAT00 | GAAGUGUGCCGUGGUGUGUCU | SEQ ID |
| miR- | 03263 | | NO: 365 |
| 595 | | | |
| hsa- | MI00036 | UACUUACUCUACGUGUGUGUCACUCGAUGACCACUGUGAAG | SEQ ID |
| miR- | 09 | ACAGUAAAAUGUACAGUGGUUCUCUUGUGGCUCAAGCGUAA | NO: 366 |
| 597 | | UGUAGAGUACUGGUC | |
| hsa- | MI00036 | GCUUGAUGAUGCUGCUGAUGCUGGCGGUGAUCCCGAUGGUG | SEQ ID |
| miR- | 10 | UGAGCUGGAAAUGGGGUGCUACGUCAUCGUUGUCAUCGUCA | NO: 367 |
| 598 | | UCAUCAUCAUCCGAG | |
| hsa- | MIMAT00 | UGGUCUAGGAUUGUUGGAGGAG | SEQ ID |
| miR- | 03269 | | NO: 368 |
| 601 | | | |
| hsa- | MIMAT00 | CACACACUGCAAUUACUUUUGC | SEQ ID |
| miR- | 03271 | | NO: 369 |
| 603 | | | |
| hsa- | MIMAT00 | AGGCUGCGGAAUUCAGGAC | SEQ ID |
| miR- | 03272 | | NO: 370 |
| 604 | | | |
| hsa- | MIMAT00 | AAACUACUGAAAAUCAAAGAU | SEQ ID |
| miR- | 03274 | | NO: 371 |
| 606 | | | |
| hsa- | MIMAT00 | GUUCAAAUCCAGAUCUAUAAC | SEQ ID |
| miR- | 03275 | | NO: 372 |
| 607 | | | |
| hsa- | MIMAT00 | AGGAAUGUUCCUUCUUUGCC | SEQ ID |
| miR- | 03281 | | NO: 373 |
| 613 | | | |
| hsa- | MIMAT00 | GAACGCCUGUUCUUGCCAGGUGG | SEQ ID |
| miR- | 03282 | | NO: 374 |
| 614 | | | |
| hsa- | MIMAT00 | UCCGAGCCUGGGUCUCCCUCUU | SEQ ID |
| miR- | 03283 | | NO: 375 |
| 615-3p | | | |
| hsa- | MIMAT00 | GGGGGUCCCCGGUGCUCGGAUC | SEQ ID |
| miR- | 04804 | | NO: 376 |
| 615-5p | | | |
| hsa- | MI00036 | UUAGGUAAUUCCUCCACUCAAAACCCUUCAGUGACUUCCAUG | SEQ ID |
| miR- | 29 | ACAUGAAAUAGGAAGUCAUUGGAGGGUUUGAGCAGAGGAAU | NO: 377 |
| 616 | | GACCUGUUUUAAAA | |
| hsa- | MIMAT00 | AGACUUCCCAUUUGAAGGUGGC | SEQ ID |
| miR- | 03286 | | NO: 378 |
| 617 | | | |
| hsa- | MIMAT00 | AAACUCUACUUGUCCUUCUGAGU | SEQ ID |
| miR- | 03287 | | NO: 379 |
| 618 | | | |
| hsa- | MI00036 | AAUGCUGUUUCAAGGUAGUACCAGUACCUUGUGUUCAGUGG | SEQ ID |
| miR- | 38 | AACCAAGGUAAACACAAGGUAUUGGUAUUACCUUGAGAUAG | NO: 380 |
| 624 | | CAUUACACCUAAGUG | |
| hsa- | MI00036 | AGGGUAGAGGGAUGAGGGGGAAAGUUCUAUAGUCCUGUAA | SEQ ID |
| miR- | 39 | UUAGAUCUCAGGACUAUAGAACUUUCCCCCUCAUCCCUCUGC | NO: 381 |
| 625 | | CCU | |
| hsa- | MIMAT00 | AGCUGUCUGAAAAUGUCUU | SEQ ID |
| miR- | 03295 | | NO: 382 |
| 626 | | | |
| hsa- | MI00036 | UACUUAUUACUGGUAGUGAGUCUCUAAGAAAAGAGGAGGUG | SEQ ID |
| miR- | 41 | GUUGUUUUCCUCCUCUUUUCUUUGAGACUCACUACCAAUAA | NO: 383 |
| 627 | | UAAGAAAUACUACUA | |
| hsa- | MIMAT00 | UCUAGUAAGAGUGGCAGUCGA | SEQ ID |
| miR- | 03297 | | NO: 384 |
| 628-3p | | | |
| hsa- | MIMAT00 | AUGCUGACAUAUUUACUAGAGG | SEQ ID |
| miR- | 04809 | | NO: 385 |
| 628-5p | | | |
| hsa- | MI00036 | UCCCUUUCCCAGGGGAGGGGCUGGGUUUACGUUGGGAGAAC | SEQ ID |
| miR- | 43 | UUUUACGGUGAACCAGGAGGUUCUCCCAACGUAAGCCCAGCC | NO: 386 |
| 629 | | CCUCCCCUCUGCCU | |
| hsa- | MIMAT00 | AGUAUUCUGUACCAGGGAAGGU | SEQ ID |
| miR- | 03299 | | NO: 387 |
| 630 | | | |
| hsa- | MIMAT00 | CUAAUAGUAUCUACCACAAUAAA | SEQ ID |
| miR- | 03303 | | NO: 388 |
| 633 | | | |
| hsa- | MIMAT00 | UGUGCUUGCUCGUCCCGCCCGCA | SEQ ID |
| miR- | 03306 | | NO: 389 |
| 636 | | | |
| hsa- | MIMAT00 | ACUGGGGGCUUUCGGGCUCUGCGU | SEQ ID |
| miR- | 03307 | | NO: 390 |
| 637 | | | |
| hsa- | MIMAT00 | AGGGAUCGCGGGCGGGUGGCGGCCU | SEQ ID |
| miR- | 03308 | | NO: 391 |
| 638 | | | |
| hsa- | MIMAT00 | AUCGCUGCGGUUGCGAGCGCUGU | SEQ ID |
| miR- | 03309 | | NO: 392 |
| 639 | | | |
| hsa- | MI00036 | AUCUGAGUUGGGAGGGUCCCUCUCCAAAUGUGUCUUGGGGU | SEQ ID |
| miR- | 57 | GGGGGAUCAAGACACAUUUGGAGAGGGAACCUCCCAACUCGG | NO: 393 |
| 642 | | CCUCUGCCAUCAUU | |
| hsa- | MIMAT00 | AGUGUGGCUUUCUUAGAGC | SEQ ID |
| miR- | 03314 | | NO: 394 |
| 644 | | | |
| hsa- | MIMAT00 | UCUAGGCUGGUACUGCUGA | SEQ ID |
| miR- | 03315 | | NO: 395 |
| 645 | | | |
| hsa- | MIMAT00 | AAGCAGCUGCCUCUGAGGC | SEQ ID |
| miR- | 03316 | | NO: 396 |
| 646 | | | |
| hsa- | MIMAT00 | AGGAGGCAGCGCUCUCAGGAC | SEQ ID |
| miR- | 03320 | | NO: 397 |
| 650 | | | |
| hsa- | MI00036 | ACGAAUGGCUAUGCACUGCACAACCCUAGGAGAGGGUGCCAU | SEQ ID |
| miR- | 67 | UCACAUAGACUAUAAUUGAAUGGCGCCACUAGGGUUGUGCA | NO: 398 |
| 652 | | GUGCACAACCUACAC | |
| hsa- | MIMAT00 | UAUGUCUGCUGACCAUCACCUU | SEQ ID |
| miR- | 04814 | | NO: 399 |
| 654-3p | | | |
| hsa- | MIMAT00 | UGGUGGGCCGCAGAACAUGUGC | SEQ ID |
| miR- | 03330 | | NO: 400 |
| 654-5p | | | |
| hsa- | MI00036 | AACUAUGCAAGGAUAUUUGAGGAGAGGUUAUCCGUGUUAUG | SEQ ID |
| miR- | 77 | UUCGCUUCAUUCAUCAUGAAUAAUACAUGGUUAACCUCUUU | NO: 401 |
| 655 | | UUGAAUAUCAGACUC | |
| hsa- | MI00036 | CUGAAAUAGGUUGCCUGUGAGGUGUUCACUUUCUAUAUGAU | SEQ ID |
| miR- | 78 | GAAUAUUAUACAGUCAACCUCUUUCCGAUAUCGAAUC | NO: 402 |
| 656 | | | |
| hsa- | MIMAT00 | GGCAGGUUCUCACCCUCUCUAGG | SEQ ID |
| miR- | 03335 | | NO: 403 |
| 657 | | | |
| hsa- | MI00036 | CUGCUCCUUCUCCCAUACCCAUUGCAUAUCGGAGUUGUGAAU | SEQ ID |
| miR- | 84 | UCUCAAAACACCUCCUGUGUGCAUGGAUUACAGGAGGGUGA | NO: 404 |
| 660 | | GCCUUGUCAUCGUG | |
| hsa- | MIMAT00 | UGCCUGGGUCUCUGGCCUGCGCGU | SEQ ID |
| miR- | 03324 | | NO: 405 |
| 661 | | | |
| hsa- | MIMAT00 | GGUGGCCCGGCCGUGCCUGAGG | SEQ ID |
| miR- | 05867 | | NO: 406 |
| 663b | | | |
| hsa- | MI00064 | GAACAUUGAAACUGGCUAGGGAAAAUGAUUGGAUAGAAACU | SEQ ID |
| miR- | 42 | AUUAUUCUAUUCAUUUAUCCCCAGCCUACAAAAUGAAAAAA | NO: 407 |
| 664 | | | |
| hsa- | MI0037 | GGUAAGUGCGCCUCGGGUGAGCAUGCACUUAAUGUGGGUGU | SEQ ID |
| miR- | 61 | AUGUCACUCGGCUCGGCCCACUACC | NO: 408 |
| 668 | | | |
| hsa- | MIMAT00 | UCCGGUUCUCAGGGCUCCACC | SEQ ID |
| miR- | 04819 | | NO: 409 |
| 671-3p | | | |
| hsa- | MIMAT00 | AGGAAGCCCUGGAGGGGCUGGAG | SEQ ID |
| miR- | 03880 | | NO: 410 |
| 671-5p | | | |
| hsa- | MI00042 | GAUGGUGAUCUAGCCCUUUAGUUUUGAGGUUGGUGUACUG | SEQ ID |
| miR- | 58 | UGUGUGAGUAUACAUAUUUAUCACACACAGUCACUAUCUUC | NO: 411 |
| 672 | | GAAAGUGAGGGUGCACAUC | |
| hsa- | N/A | GCACUGAGAUGGGAGUGGUGUA | SEQ ID |
| miR- | | | NO: 412 |
| 674 | | | |
| hsa- | MI00055 | AACUGCCCUCAAGGAGCUUACAAUCUAGCUGGGGGUAAAUG | SEQ ID |
| miR- | 43 | ACUUGCACAUGAACACAACUAGACUGUGAGCUUCUAGAGGGC | NO: 413 |
| 708 | | AGGGA | |
| hsa- | IMAT000 | UCUCGCUGGGGCCUCCA | SEQ ID |
| miR- | 5954 | | NO: 414 |
| 720 | | | |
| hsa- | MI00055 | UUGGGCAAGGUGCGGGGCUAGGGCUAACAGCAGUCUUACUG | SEQ ID |
| miR- | 59 | AAGGUUUCCUGGAAACCACGCACAUGCUGUUGCCACUAACCU | NO: 415 |
| 744 | | CAACCUUACUCGGUC | |
| hsa- | MI00037 | GCCUGGAUACAUGAGAUGGUUGACCAGAGAGCACACGCUUU | SEQ ID |
| miR- | 57 | AUUUGUGCCGUUUGUGACCUGGUCCACUAACCCUCAGUAUC | NO: 416 |
| 758 | | UAAUGC | |
| hsa- | MIMAT00 | UGGAGGAGAAGGAAGGUGAUG | SEQ ID |
| miR- | 03945 | | NO: 417 |
| 765 | | | |
| hsa- | MI00038 | GCAUCCUCAGGACCUGGGCUUGGGUGGUAGGAGGAAUUGGU | SEQ ID |
| miR- | 36 | GCUGGUCUUUCAUUUUGGAUUUGACUCCAGCCCCACAGCCUC | NO: 418 |
| 766 | | AGCCACCCCAGCCAAUUGUCAUAGGAGC | |
| hsa- | MIMAT00 | UGCACCAUGGUUGUCUGAGCAUG | SEQ ID |
| miR- | 03882 | | NO: 419 |
| 767-5p | | | |
| hsa- | MIMAT00 | UGAGACCUCUGGGUUCUGAGCU | SEQ ID |
| miR- | 03886 | | NO: 420 |
| 769-5p | | | |
| hsa- | MIMAT00 | UCCAGUACCACGUGUCAGGGCCA | SEQ ID |
| miR- | 03948 | | NO: 421 |
| 770-5p | | | |
| hsa- | N/A | AAGGUUACUUGUUAGUUCAGG | SEQ ID |
| miR- | | | NO: 422 |
| 872 | | | |
| hsa- | MI00055 | UUAGCCCUGCGGCCCCACGCACCAGGGUAAGAGAGACUCUCG | SEQ ID |
| miR- | 32 | CUUCCUGCCCUGGCCCGAGGGACCGACUGGCUGGGC | NO: 423 |
| 874 | | | |
| hsa- | MIMAT00 | UAUACCUCAGUUUUAUCAGGUG | SEQ ID |
| miR- | 04922 | | NO: 424 |
| 875-5p | | | |
| hsa- | MIMAT00 | UCCAUUACACUACCCUGCCUCU | SEQ ID |
| miR- | 04947 | | NO: 425 |
| 885-5p | | | |
| hsa- | MIMAT00 | CGCGGGUGCUUACUGACCCUU | SEQ ID |
| miR- | 04906 | | NO: 426 |
| 886-3p | | | |
| hsa- | MIMAT00 | CGGGUCGGAGUUAGCUCAAGCGG | SEQ ID |
| miR- | 04905 | | NO: 427 |
| 886-5p | | | |
| hsa- | MI00055 | GGCAGUGCUCUACUCAAAAAGCUGUCAGUCACUUAGAUUACA | SEQ ID |
| miR- | 37 | UGUGACUGACACCUCUUUGGGUGAAGGAAGGCUCA | NO: 428 |
| 888 | | | |
| hsa- | MI00055 | GUGCUUAAAGAAUGGCUGUCCGUAGUAUGGUCUCUAUAUUU | SEQ ID |
| miR- | 40 | AUGAUGAUUAAUAUCGGACAACCAUUGUUUUAGUAUCC | NO: 429 |
| 889 | | | |
| hsa- | MIMAT00 | UACUUGGAAAGGCAUCAGUUG | SEQ ID |
| miR- | 04912 | | NO: 430 |
| 890 | | | |
| hsa- | MIMAT00 | CACUGGCUCCUUUCUGGGUAGA | SEQ ID |
| miR- | 04918 | | NO: 431 |
| 892b | | | |
| hsa- | MI00004 | CGGGGUUGGUUGUUAUCUUUGGUUAUCUAGCUGUAUGAGU | SEQ ID |
| miR-9 | 66 | GGUGUGGAGUCUUCAUAAAGCUAGAUAACCGAAAGUAAAAA | NO: 432 |
| | | UAACCCCA | |
| hsa- | MIMAT00 | AGAGUCUUGUGAUGUCUUGC | SEQ ID |
| miR- | 04974 | | NO: 433 |
| 924 | | | |
| hsa- | MI00000 | CUUUCUACACAGGUUGGGAUCGGUUGCAAUGCUGUGUUUCU | SEQ ID |
| miR- | 93 | GUAUGGUAUUGCACUUGUCCCGGCCUGUUGAGUUUGG | NO: 434 |
| 92a | | | |
| hsa- | MI00000 | CUGGGGGCUCCAAAGUGCUGUUCGUGCAGGUAGUGUGAUUA | SEQ ID |
| miR-93 | 95 | CCCAACCUACUGCUGAGCUAGCACUUCCCGAGCCCCCGG | NO: 435 |
| hsa- | MIMAT00 | CCAGUUACCGCUUCCGCUACCGC | SEQ ID |
| miR- | 04978 | | NO: 436 |
| 935 | | | |
| hsa- | MI00057 | AGCACUGCCCCCGGUGAGUCAGGGUGGGGCUGGCCCCCUGCU | SEQ ID |
| miR- | 59 | UCGUGCCCAUCCGCGCUCUGACUCUCUGCCCACCUGCAGGAG | NO: 437 |
| 937 | | CU | |
| hsa- | MI00057 | UGUGGGCAGGGCCCUGGGGAGCUGAGGCUCUGGGGGUGGCC | SEQ ID |
| miR- | 61 | GGGGCUGACCCUGGGCCUCUGCUCCCCAGUGUCUGACCGCG | NO: 438 |
| 939 | | | |
| hsa- | MI00057 | AUUAGGAGAGUAUCUUCUCUGUUUUGGCCAUGUGUGUACUC | SEQ ID |
| miR- | 67 | ACAGCCCCUCACACAUGGCCGAAACAGAGAAGUUACUUUCCU | NO: 439 |
| 942 | | AAU | |
| hsa- | MIMAT00 | CUGACUGUUGCCGUCCUCCAG | SEQ ID |
| miR- | 04986 | | NO: 440 |
| 943 | | | |
| hsa- | MI00000 | AACACAGUGGGCACUCAAUAAAUGUCUGUUGAAUUGAAAUG | SEQ ID |
| miR-95 | 97 | CGUUACAUUCAACGGGUAUUUAUUGAGCACCCACUCUGUG | NO: 441 |
| hsa- | MI00000 | UGGCCGAUUUUGGCACUAGCACAUUUUUGCUUGUGUCUCUC | SEQ ID |
| miR-96 | 98 | CGCUCUGAGCAAUCAUGUGCAGUGCCAAUAUGGGAAA | NO: 442 |
| hsa- | MI00001 | AGGAUUCUGCUCAUGCCAGGGUGAGGUAGUAAGUUGUAUU | SEQ ID |
| miR-98 | 00 | GUUGUGGGGUAGGGAUAUUAGGCCCCAAUUAGAAGAUAACU | NO: 443 |
| | | AUACAACUUACUACUUUCCCUGGUGUGUGGCAUAUUCA | |
| hsa- | MI00001 | CCCAUUGGCAUAAACCCGUAGAUCCGAUCUUGUGGUGAAGU | SEQ ID |
| miR- | 01 | GGACCGCACAAGCUCGCUUCUAUGGGUCUGUGUCAGUGUG | NO: 444 |
| 99a | | | |
| hsa- | MI00007 | GGCACCCACCCGUAGAACCGACCUUGCGGGGCCUUCGCCGCAC | SEQ ID |
| miR- | 46 | ACAAGCUCGUGUCUGUGGGUCCGUGUC | NO: 445 |
| 99b | | | |
| Mamm | N/A | GUGCUCGCUUCGGCAGCACAUAUACUAAAAUUGGAACGAUAC | SEQ ID |
| U6 | | AGAGAAGAUUAGCAUGGCCCCUGCGCAAGGAUGACACGCAAA | NO: 446 |
| | | UUCGUGAAGCGUUCCAUAUUUUUACUGCCCUCCAUGCCCUGC | |
| | | CCCACAAACGCUCUGAUAACAGUCUGUCCCUGUCUCUCUCCU | |
| | | GCUGCUCCUAUGGAAGCGAAGUUUUCCGCUCCUGCAGAAAGC | |
| | | AAAGUUACGACUCAGAGACGGCUGAGGAUGACAUCAGCGAU | |
| | | GUGCAGGGAACCCAGCGCCUGGAGCUUCGGGAUGACGGGGC | |
| | | CUUCAGCACCCCCACGGGGGGUUCUGACACCCUGGUGGGCAC | |
| | | CUCCCUGGACACACCCCCGACCUCCGUGACAGGCACCUCAGAG | |
| | | GAGCAAGUGAGCUGGUGGGGCAGCGGGCAGACGGUCCUGGA | |
| | | GCAGGAAGCGGGCAGUGGGGGUGGCACCCGCCGCCUCCCGGG | |
| | | CAGCCCAAGGCAAGCACAGGCAACCGGGGCCGGGCCACGGCAC | |
| | | CUGGGGGUGGAGCCGCUGGUGCGGGCAUCUCGAGCUAAUCU | |
| | | GGUGGG | |
| miR- | JX991307 | GUCUUUGCACCAUCUCUGAAAAGCCGAUGUGUAUCCUCAGCU | SEQ ID |
| 146a | | UUGAGAACUGAAUUCCAUGGGUUGUGUCAGUGUCAGACCUC | NO: 447 |
| | | UGAAAUUCAGUUCUUCAGCUGGGAUAUCUCUGUCAUCGUGG | |
| | | GCUUGAGGACCUGGAGAGAGUAGAUCCUGAAGAACUUUUUC | |
| | | AGUCUGCUGAAGAGCUUGGAAGACUGGAGACAGAAGGCAGA | |
| | | GUCUCAGGCUCUGAAGGUAUAAGGAGUGUGAGUUCCUGUGA | |
| | | GAAACACUCAUUUGAUUGU | |
| miR- | NR_0301 | CCUGGCACUGAGAACUGAAUUCCAUAGGCUGUGAGCUCUAG | SEQ ID |
| 146b | 69 | CAAUGCCCUGUGGACUCAGUUCUGGUGCCCGG | NO: 448 |
| miR- | hsa-mir- | CCUGGCACUGAGAACUGAAUUCCAUAGGCUGUGAGCUCUAG | SEQ ID |
| 146b | 146bM10 | CAAUGCCCUGUGGACUCAGUUCUGGUGCCCGG | NO: 449 |
| | 003129 | | |
| miR- | | AGCUACAUCUGGCUACUGGGU | SEQ ID |
| 222 | | | NO: 450 |
| miR- | hsa-miR- | UGCCUACUGAGCUGAAACACAG | SEQ ID |
| 24-2# | 24-2- | | NO: 451 |
| | 5pMIMAT | | |
| | 0004497 | | |
| miR- | | UUCACAGUGGCUAAGUUCCGC | SEQ ID |
| 27a | | | NO: 452 |
| miR- | NR_0298 | GAAACUGGGCUCAAGGUGAGGGGUGCUAUCUGUGAUUGAG | SEQ ID |
| 342-3p | 88 | GGACAUGGUUAAUGGAAUUGUCUCACACAGAAAUCGCACCCG | NO: 453 |
| | | UCACCUUGGCCUACUUA | |
| mmu- | MI00005 | UUCACUGUGGGAUGAGGUAGUAGGUUGUAUAGUUUUAGGG | SEQ ID |
| let-7a-1 | 56 | UCACACCCACCACUGGGAGAUAACUAUACAAUCUACUGUCUU | NO: 454 |
| | | UCCUAAGGUGAU | |
| mmu- | MI00005 | CUGCAUGUUCCCAGGUUGAGGUAGUAGGUUGUAUAGUUUA | SEQ ID |
| let-7a-2 | 57 | GAGUUACAUCAAGGGAGAUAACUGUACAGCCUCCUAGCUUU | NO: 455 |
| | | CCUUGGGACUUGCAC | |
| mmu- | MI00005 | GCAGGGUGAGGUAGUAGGUUGUGUGGUUUCAGGGCAGUGA | SEQ ID |
| let-7b | 58 | UGUUGCCCCUCCGAAGAUAACUAUACAACCUACUGCCUUCCC | NO: 456 |
| | | UGA | |
| mmu- | MI00005 | UGUGUGCAUCCGGGUUGAGGUAGUAGGUUGUAUGGUUUAG | SEQ ID |
| let-7c-1 | 59 | AGUUACACCCUGGGAGUUAACUGUACAACCUUCUAGCUUUCC | NO: 457 |
| | | UUGGAGCACACU | |
| mmu- | MI00005 | ACGGCCUUUGGGGUGAGGUAGUAGGUUGUAUGGUUUUGGG | SEQ ID |
| let-7c-2 | 60 | CUCUGCCCCGCUCUGCGGUAACUAUACAAUCUACUGUCUUUC | NO: 458 |
| | | CUGAAGUGGCCGC | |
| mmu- | MI00004 | AAUGGGUUCCUAGGAAGAGGUAGUAGGUUGCAUAGUUUUA | SEQ ID |
| let-7d | 05 | GGGCAGAGAUUUUGCCCACAAGGAGUUAACUAUACGACCUGC | NO: 459 |
| | | UGCCUUUCUUAGGGCCUUAUU | |
| mmu- | MI00005 | CGCGCCCCCCGGGCUGAGGUAGGAGGUUGUAUAGUUGAGGA | SEQ ID |
| let-7e | 61 | AGACACCCGAGGAGAUCACUAUACGGCCUCCUAGCUUUCCCC | NO: 460 |
| | | AGGCUGCGCC | |
| mmu- | MI00005 | AUCAGAGUGAGGUAGUAGAUUGUAUAGUUGUGGGGUAGUG | SEQ ID |
| let-7f | 62 | AUUUUACCCUGUUUAGGAGAUAACUAUACAAUCUAUUGCCU | NO: 461 |
| | | UCCCUGAG | |
| mmu- | MI00005 | UGUGGGAUGAGGUAGUAGAUUGUAUAGUUUUAGGGUCAUA | SEQ ID |
| let-7f-2 | 63 | CCCCAUCUUGGAGAUAACUAUACAGUCUACUGUCUUUCCCAC | NO: 462 |
| | | G | |
| mmu- | MI00001 | CCAGGCUGAGGUAGUAGUUUGUACAGUUUGAGGGUCUAUG | SEQ ID |
| let-7g | 37 | AUACCACCCGGUACAGGAGAUAACUGUACAGGCCACUGCCUU | NO: 463 |
| | | GCCAGG | |
| mmu- | MI00001 | CUGGCUGAGGUAGUAGUUUGUGCUGUUGGUCGGGUUGUGA | SEQ ID |
| let-7i | 38 | CAUUGCCCGCUGUGGAGAUAACUGCGCAAGCUACUGCCUUGC | NO: 464 |
| | | UAG | |
| mmu- | MI00001 | GCUUGGGACACAUACUUCUUUAUAUGCCCAUAUGAACCUGC | SEQ ID |
| miR-1 | 39 | UAAGCUAUGGAAUGUAAAGAAGUAUGUAUUUCAGGC | NO: 465 |
| mmu- | N/A | UACAUACUUCUUUACAUUCCA | SEQ ID |
| miR-1- | | | NO: 466 |
| 2-as | | | |
| mmu- | MI00006 | CCUGUUGCCACAAACCCGUAGAUCCGAACUUGUGCUGAUUCU | SEQ ID |
| miR- | 92 | GCACACAAGCUUGUGUCUAUAGGUAUGUGUCUGUUAGG | NO: 467 |
| 100 | | | |
| mmu- | MI00001 | AGGCUGCCCUGGCUCAGUUAUCACAGUGCUGAUGCUGUCCA | SEQ ID |
| miR- | 48 | UUCUAAAGGUACAGUACUGUGAUAACUGAAGGAUGGCAGCC | NO: 468 |
| 101a | | A | |
| mmu- | MI00006 | AUCUGAGACUGAACUGCCCUUUUUCGGUUAUCAUGGUACCG | SEQ ID |
| miR- | 49 | AUGCUGUAGCUCUGAAAGGUACAGUACUGUGAUAGCUGAAG | NO: 469 |
| 101b | | AAUGGCGGUGCCAUC | |
| mmu- | MI00005 | UUCUUACUGCCCUCGGCUUCUUUACAGUGCUGCCUUGUUGC | SEQ ID |
| miR- | 87 | AUAUGGAUCAAGCAGCAUUGUACAGGGCUAUGAAGGCAUUG | NO: 470 |
| 103 | | AGAC | |
| mmu- | MIMAT00 | CCAAGUGCUCAGAUGCUUGUGGU | SEQ ID |
| miR- | 04856 | | NO: 471 |
| 105 | | | |
| mmu- | MI00004 | AUGUCAAAGUGCUAACAGUGCAGGUAGCUUUUUGAGUUCUA | SEQ ID |
| miR- | 06 | CUGCAGUGCCAGCACUUCUUACAU | NO: 472 |
| 106a | | | |
| mmu- | MI00004 | CCUGCUGGGACUAAAGUGCUGACAGUGCAGAUAGUGGUCCU | SEQ ID |
| miR- | 07 | CUCUGUGCUACCGCACUGUGGGUACUUGCUGCUCCAGCAGG | NO: 473 |
| 106b | | | |
| mmu- | MI00006 | UUCUCUGUGCUUUCAGCUUCUUUACAGUGUUGCCUUGUGGC | SEQ ID |
| miR- | 84 | AUGGAGUUCAAGCAGCAUUGUACAGGGCUAUCAAAGCACAG | NO: 474 |
| 107 | | AGAGC | |
| mmu- | MI00006 | GACCUGUCUGUCUUCUGUAUAUACCCUGUAGAUCCGAAUUU | SEQ ID |
| miR- | 85 | GUGUAAGGAAUUUUGUGGUCACAAAUUCGUAUCUAGGGGAA | NO: 475 |
| 10a | | UAUGUAGUUGACAUAAACACUCCGCUCA | |
| mmu- | MI00002 | UAUAUACCCUGUAGAACCGAAUUUGUGUGGUACCCACAUAG | SEQ ID |
| miR- | 21 | UCACAGAUUCGAUUCUAGGGGAAUAUA | NO: 476 |
| 10b | | | |
| mmu- | N/A | GAGUGCUGGAAUUAAAGGCAUG | SEQ ID |
| miR- | | | NO: 477 |
| 1186 | | | |
| mmu- | MI00062 | AUACUCACAGUCUCCCAGCUGGUGUGAGGUUGGGCCAGGAU | SEQ ID |
| miR- | 90 | GAAACCCAAGGCUCUCCGAGGCUCCCCACCACACCCUGCUGCU | NO: 478 |
| 1188 | | GAAGACUGCCUAGCAAGGCUGUGCCGAGUGGUGUGG | |
| mmu- | MIMAT00 | CAGUCUUACUAUGUAGCCCUA | SEQ ID |
| miR- | 05849 | | NO: 479 |
| 1191 | | | |
| mmu- | MIMAT00 | GAAUGAGUAACUGCUAGAUCCU | SEQ ID |
| miR- | 05852 | | NO: 480 |
| 1194 | | | |
| mmu- | MIMAT00 | UGAGUUCGAGGCCAGCCUGCUCA | SEQ ID |
| miR- | 05856 | | NO: 481 |
| 1195 | | | |
| mmu- | MI00063 | UUUUUUUUCUUUUGUUUAUUUUGUUUGUGACAGUUGUCU | SEQ ID |
| miR- | 06 | GUUAUGUGUUCCUGGCUGGCUUGGUACUCAUGUGUAGCCCA | NO: 482 |
| 1198 | | AGCUAGCCUCUAACUCAUGGCAGUCAUCCUGUCUCAGUCUC | |
| mmu- | MI00002 | AGCUGUGGAGUGUGACAAUGGUGUUUGUGUCCAAACCAUCA | SEQ ID |
| miR- | 56 | AACGCCAUUAUCACACUAAAUAGCU | NO: 483 |
| 122 | | | |
| mmu- | MI00007 | AGGCCUCUCUCUCCGUGUUCACAGCGGACCUUGAUUUAAAU | SEQ ID |
| miR- | 16 | GUCCAUACAAUUAAGGCACGCGGUGAAUGCCAAGAAUGGGG | NO: 484 |
| 124 | | CUG | |
| mmu- | MIMAT00 | ACAGGUGAGGUUCUUGGGAGCC | SEQ ID |
| miR- | 04528 | | NO: 485 |
| 125a- | | | |
| 3p | | | |
| mmu- | MIMAT00 | UCCCUGAGACCCUUUAACCUGUGA | SEQ ID |
| miR- | 00135 | | NO: 486 |
| 125a- | | | |
| 5p | | | |
| mmu- | MI00007 | UGCGCUCCCCUCAGUCCCUGAGACCCUAACUUGUGAUGUUUA | SEQ ID |
| miR- | 25 | CCGUUUAAAUCCACGGGUUAGGCUCUUGGGAGCUG | NO: 487 |
| 125b | | | |
| mmu- | MI00001 | GCCUAGUCCCUGAGACCCUAACUUGUGAGGUAUUUUAGUAA | SEQ ID |
| miR- | 52 | CAUCACAAGUCAGGUUCUUGGGACCUAGGC | NO: 488 |
| 125b-2 | | | |
| mmu- | MIMAT00 | UCCCUGAGACCCUAACUUGUGA | SEQ ID |
| miR- | 00136 | | NO: 489 |
| 125b- | | | |
| 5p | | | |
| mmu- | MI00001 | UGACAGCACAUUAUUACUUUUGGUACGCGCUGUGACACUUC | SEQ ID |
| miR- | 53 | AAACUCGUACCGUGAGUAAUAAUGCGCGGUCA | NO: 490 |
| 126 | | | |
| mmu- | MIMAT00 | UCGUACCGUGAGUAAUAAUGCG | SEQ ID |
| miR- | 00138 | | NO: 491 |
| 126a- | | | |
| 3p | | | |
| mmu- | MIMAT00 | CAUUAUUACUUUUGGUACGCG | SEQ ID |
| miR- | 00137 | | NO: 492 |
| 126a- | | | |
| 5p | | | |
| mmu- | MI00001 | CCAGCCUGCUGAAGCUCAGAGGGCUCUGAUUCAGAAAGAUCA | SEQ ID |
| miR- | 54 | UCGGAUCCGUCUGAGCUUGGCUGGUCGG | NO: 493 |
| 127 | | | |
| mmu- | MIMAT00 | UCAGGUCCCUGUUCAGGCGCCA | SEQ ID |
| miR- | 09445 | | NO: 494 |
| 1274a | | | |
| mmu- | MIMAT00 | UCACAGUGAACCGGUCUCUUU | SEQ ID |
| miR- | 00140 | | NO: 495 |
| 128a | | | |
| mmu- | MIMAT00 | AAGCCCUUACCCCAAAAAGUAU | SEQ ID |
| miR- | 16994 | | NO: 496 |
| 129-3p | | | |
| mmu- | MI00099 | CAGUCUCCACCACCUCCCCUGCAAACGUCCAGUGAUGCAGAG | SEQ ID |
| miR- | 35 | GUAAUGGACGUUGGCUCUGGUGGUGAUGGACAGUCCG | NO: 497 |
| 1306 | | | |
| mmu- | MI00001 | GAGCUCUUUUCACAUUGUGCUACUGUCUAACGUGUACCGAG | SEQ ID |
| miR- | 56 | CAGUGCAAUGUUAAAAGGGCAUC | NO: 498 |
| 130a | | | |
| mmu- | MI00004 | GGCUUGUUGGACACUCUUUCCCUGUUGCACUACUGUGGGCC | SEQ ID |
| miR- | 08 | UCUGGGAAGCAGUGCAAUGAUGAAAGGGCAUCUGUCGGGCC | NO: 499 |
| 130b | | | |
| mmu- | MI00001 | GGGCAACCGUGGCUUUCGAUUGUUACUGUGGGAACCGGAGG | SEQ ID |
| miR- | 58 | UAACAGUCUACAGCCAUGGUCGCCC | NO: 500 |
| 132 | | | |
| mmu- | MI00001 | GCUAAAGCUGGUAAAAUGGAACCAAAUCGCCUCUUCAAUGGA | SEQ ID |
| miR- | 59 | UUUGGUCCCCUUCAACCAGCUGUAGC | NO: 501 |
| 133a | | | |
| mmu- | MI00008 | CCUCCAAAGGGAGUGGCCCCCUGCUCUGGCUGGUCAAACGGA | SEQ ID |
| miR- | 21 | ACCAAGUCCGUCUUCCUGAGAGGUUUGGUCCCCUUCAACCAG | NO: 502 |
| 133b | | CUACAGCAGGGCUGGCAAAGCUCAAUAUUUGGAGA | |
| mmu- | MI00001 | AGGCCUCACUGUUCUCUAUGGCUUUUUAUUCCUAUGUGAUU | SEQ ID |
| miR- | 61 | CUAUUGCUCGCUCAUAUAGGGAUUGGAGCCGUGGCGUACGG | NO: 503 |
| 135a-1 | | UGAGGAUA | |
| mmu- | MI00007 | AGAUAAAUUCACUCUAGUGCUUUAUGGCUUUUUAUUCCUAU | SEQ ID |
| miR- | 15 | GUGAUCGUAAUAAAGUCUCAUGUAGGGAUGGAAGCCAUGAA | NO: 504 |
| 135a-2 | | AUACAUUGUGAAAAUUCA | |
| mmu- | MI00006 | CGCUCUGCUGUGGCCUAUGGCUUUUCAUUCCUAUGUGAUUG | SEQ ID |
| miR- | 46 | CUGCUCCGAACUCAUGUAGGGCUAAAAGCCAUGGGCUACAGU | NO: 505 |
| 135b | | GAGGGGCAAGCUCC | |
| mmu- | MI00001 | CUUCGGUGACGGGUAUUCUUGGGUGGAUAAUACGGAUUACG | SEQ ID |
| miR- | 63 | UUGUUAUUGCUUAAGAAUACGCGUAGUCGAGG | NO: 506 |
| 137 | | | |
| mmu- | MI00007 | CUCUAGCAUGGUGUUGUGGGACAGCUGGUGUUGUGAAUCA | SEQ ID |
| miR- | 22 | GGCCGUUGCCAAUCAGAGAACGGCUACUUCACAACACCAGGG | NO: 507 |
| 138 | | CCACACUGCACUGCAAG | |
| mmu- | MIMAT00 | UGGAGACGCGGCCCUGUUGGAG | SEQ ID |
| miR- | 04662 | | NO: 508 |
| 139-3p | | | |
| mmu- | MIMAT00 | UCUACAGUGCACGUGUCUCCAG | SEQ ID |
| miR- | 00656 | | NO: 509 |
| 139-5p | | | |
| mmu- | MI00001 | CCUGCCAGUGGUUUUACCCUAUGGUAGGUUACGUCAUGCUG | SEQ ID |
| miR- | 65 | UUCUACCACAGGGUAGAACCACGGACAGG | NO: 510 |
| 140 | | | |
| mmu- | MI00001 | GGGUCCAUCUUCCAGUGCAGUGUUGGAUGGUUGAAGUAUGA | SEQ ID |
| miR- | 66 | AGCUCCUAACACUGUCUGGUAAAGAUGGCCC | NO: 511 |
| 141 | | | |
| mmu- | MI00001 | ACCCAUAAAGUAGAAAGCACUACUAACAGCACUGGAGGGUGU | SEQ ID |
| miR- | 67 | AGUGUUUCCUACUUUAUGGAUG | NO: 512 |
| 142 | | | |
| mmu- | MIMAT00 | UGUAGUGUUUCCUACUUUAUGGA | SEQ ID |
| miR- | 00155 | | NO: 513 |
| 142-3p | | | |
| mmu- | MIMAT00 | CAUAAAGUAGAAAGCACUACU | SEQ ID |
| miR- | 00154 | | NO: 514 |
| 142-5p | | | |
| mmu- | MI00002 | CCUGAGGUGCAGUGCUGCAUCUCUGGUCAGUUGGGAGUCUG | SEQ ID |
| miR- | 57 | AGAUGAAGCACUGUAGCUCAGG | NO: 515 |
| 143 | | | |
| mmu- | MI00001 | CUCACGGUCCAGUUUUCCCAGGAAUCCCUUGGAUGCUAAGAU | SEQ ID |
| miR- | 69 | GGGGAUUCCUGGAAAUACUGUUCUUGAG | NO: 516 |
| 145 | | | |
| mmu- | MI00001 | AGCUCUGAGAACUGAAUUCCAUGGGUUAUAUCAAUGUCAGA | SEQ ID |
| miR- | 70 | CCUGUGAAAUUCAGUUCUUCAGCU | NO: 517 |
| 146 | | | |
| mmu- | MI00046 | GACUGAGAGAACUUUGGCCACCUGGCUCUGAGAACUGAAUU | SEQ ID |
| miR- | 65 | CCAUAGGCUGUGAGCUCUAGCAGACGCCCUAGGGACUCAGUU | NO: 518 |
| 146b | | CUGGUGCCCGGCUGUGCUAUACCAUC | |
| mmu- | MI00005 | AGCCAGUUUGGUCUUUUGAGACAAAGUUCUGAGACACUCCG | SEQ ID |
| miR- | 50 | ACUCUGAGUAUGAUAGAAGUCAGUGCACUACAGAACUUUGU | NO: 519 |
| 148a | | CUCUAGAGGCUGUGGUC | |
| mmu- | MI00006 | CAGGCACCCUUAGCAUUUGAGGUGAAGUUCUGUUAUACACU | SEQ ID |
| miR- | 17 | CAGGCUGUGGCUCUGAAAGUCAGUGCAUCACAGAACUUUGU | NO: 520 |
| 148b | | CUCGAAAGCUUUCUA | |
| mmu- | MI00001 | GGCUCUGGCUCCGUGUCUUCACUCCCGUGUUUGUCCGAGGA | SEQ ID |
| miR- | 71 | GGGAGGGAGGGACGGGGGCGGUGCU | NO: 521 |
| 149 | | | |
| mmu- | MI00001 | CCCUGUCUCCCAACCCUUGUACCAGUGCUGUGCCUCAGACCC | SEQ ID |
| miR- | 72 | UGGUACAGGCCUGGGGGAUAGGG | NO: 522 |
| 150 | | | |
| mmu- | MIMAT00 | CUAGACUGAGGCUCCUUGAGG | SEQ ID |
| miR- | 00161 | | NO: 523 |
| 151-3p | | | |
| mmu- | MI00001 | CCGGGCCUAGGUUCUGUGAUACACUCCGACUCGGGCUCUGG | SEQ ID |
| miR- | 74 | AGCAGUCAGUGCAUGACAGAACUUGGGCCCGG | NO: 524 |
| 152 | | | |
| mmu- | MI00001 | CUGUUAAUGCUAAUUGUGAUAGGGGUUUUGGCCUCUGACU | SEQ ID |
| miR- | 77 | GACUCCUACCUGUUAGCAUUAACAG | NO: 525 |
| 155 | | | |
| mmu- | MI00005 | CCCUUGGAGUAAAGUAGCAGCACAUAAUGGUUUGUGGAUGU | SEQ ID |
| miR- | 64 | UGAAAAGGUGCAGGCCAUACUGUGCUGCCUCAAAAUACAAGG | NO: 526 |
| 15a | | A | |
| mmu- | MI00001 | CUGUAGCAGCACAUCAUGGUUUACAUACUACAGUCAAGAUGC | SEQ ID |
| miR- | 40 | GAAUCAUUAUUUGCUGCUCUAG | NO: 527 |
| 15b | | | |
| mmu- | MI00005 | AUGUCAGCGGUGCCUUAGCAGCACGUAAAUAUUGGCGUUAA | SEQ ID |
| miR-16 | 65 | GAUUCUGAAAUUACCUCCAGUAUUGACUGUGCUGCUGAAGU | NO: 528 |
| | | AAGGUUGGCAA | |
| mmu- | MI00006 | GUCAGAAUAAUGUCAAAGUGCUUACAGUGCAGGUAGUGAUG | SEQ ID |
| miR-17 | 87 | UGUGCAUCUACUGCAGUGAGGGCACUUGUAGCAUUAUGCUG | NO: 529 |
| | | AC | |
| mmu- | MGI1923 | CCUAGUCAUACACGUGGACCUAGCAGCACCCGAAGCAUUGCC | SEQ ID |
| miR- | 207 | CAAGGAUACUUGCUGAGAAGGAAGUUUGCCUGAGUGGGUGA | NO: 530 |
| 17hg | | GUAUAUUCUAGUUUUGUAGCUAAAACUUCUUUUGAGACCUU | |
| | | UGGUUUUCACUUUUGUCUUUGAGCUUGUACAACAUUCUUG | |
| | | GUCUUUUAAGGUAUGAAAUAAAUACAGUUUGAACUCUUGUC | |
| | | AUAAAUGAGUGAGCCACAUUUUAACGUAGUAAAUCUACAGU | |
| | | GGCUUUUGGACACUAACCAAAUAGUUGUAGACCCUUCAAGU | |
| | | UGGAGUCAUAGAGUAUUUCUAAUUUGGGGUGAUACUAAGAC | |
| | | UUUUUUAUGUUUUAUGACUAAUAAACUUGAAAAUGACUAAA | |
| | | CAAUAAUCAUUAAUCUUGUCGAGUAUCUGACAAUGUGGAGG | |
| | | ACAGAAGAAAGGGAUUGCUGCCUGGUCAAUGUGAGGCUUUC | |
| | | CUCUAAAGGUAGUACCAAGUCAGACUGCCUUCCUGAUAACAG | |
| | | GUCUCAUUUUGUAGGACCCUUAUUGUGUGUUUUUUGGGGA | |
| | | AGCCUACUGUAAAAGCCAACAUUUUAAUGGGAUUGUAUCUU | |
| | | AUAUUUCUUUAAGGAGUUUUUUUUUUUUUUUUAAGGCUUA | |
| | | CAUGUGUCCAAUUUGGAACUUCUGGCUAUUGGCUCCUCCCC | |
| | | UCCCCCCCUUGGGUAUAAGCUGUAAUUGAUGUUUGUGACAG | |
| | | AAUUUAGAGCUUUGGCUUUUUCCUUUUUGUCUAAUUAUUU | |
| | | AUUUCAAAUUUAGCAGGAAUAAAGUGAACCUCACCUUGGGA | |
| | | CUGAAGCUGUGACCAGUCAGAAUAAUGUCAAAGUGCUUACA | |
| | | GUGCAGGUAGUGAUGUGUGCAUCUACUGCAGUGAGGGCACU | |
| | | UGUAGCAUUAUGCUGACAGCUGCCUCGGUGGGAGCCACAGU | |
| | | GGGCGCUGCCUCGGGCGGCACUGGCUGCGUCCAGUCGUCGG | |
| | | UCAGUCGGUCGCGGGGAGGGCCUGCUGGUGCUGCGUGCUUU | |
| | | UUGUUCUAAGGUGCAUCUAGUGCAGAUAGUGAAGUAGACUA | |
| | | GCAUCUACUGCCCUAAGUGCUCCUUCUGGCAUAAGAAGUUA | |
| | | UGUCCUCAUCCAAUCCAAGUCAAGCAAGCAUGUAGGGGUCUC | |
| | | UCCAUAGUUGUGUUUGCAGCCCUCUGUUAGUUUUGCAUAGU | |
| | | UGCACUACAAGAAGAAUGUAGUUGUGCAAAUCUAUGCAAAA | |
| | | CUGAUGGUGGCCUGCUAUUUACUUCAAGUGUUGUUUUUUU | |
| | | UUAAACUAAUUUUGUAUUUUUAUUGUGUCGAUGUAGAGCC | |
| | | UGCGUGGUGUGUGUGAUGUGACAGCUUCUGUAGCACUAAAG | |
| | | UGCUUAUAGUGCAGGUAGUGUGUAGCCAUCUACUGCAUUAC | |
| | | GAGCACUUAAAGUACUGCCAGCUGUAGAACUCCAGCCUCGCC | |
| | | UGGCCAUCGCCCAGCCAACUGUCCUGUUAUUGAGCACUGGUC | |
| | | UAUGGUUAGUUUUGCAGGUUUGCAUCCAGCUGUAUAAUAU | |
| | | UCUGCUGUGCAAAUCCAUGCAAAACUGACUGUGGUGGUGAA | |
| | | AAGUCUGUAGAGAAGUAAGGGAAAAUCAAACCCCUUUCUACA | |
| | | CAGGUUGGGAUUUGUCGCAAUGCUGUGUUUCUCUGUAUGG | |
| | | UAUUGCACUUGUCCCGGCCUGUUGAGUUUGGUGGGGAUUG | |
| | | UGACCAGAAGAUGUGAAAAUGACAAUAUUGCUGAAGAUGCC | |
| | | GAUUUCCACUGUAAAAAUGUUCACGAGCUGAUGGAGUGUAU | |
| | | GAACACAUUCUGUAAACUGUGAUUUCUAUUGCUUUCGUUGG | |
| | | UGUGUAAAACGGGAUUUUUUUAAUUGAAAUUUGUGUUUUU | |
| | | AAAUUGUGGUUUGUUUUUACUGAAUCAUUUCAUUUUCGUG | |
| | | UUUGCUCGCUGGAUUUAGUAAGAAGUUGUGUGUGGGGGCU | |
| | | UAGGUGAAGUUAAACUGCCUUUUGAACCUGGUAUGACUUCA | |
| | | GGUUUAUUUUACUCUAAAACCCAUUAACAUGAGUUAAGAAA | |
| | | AAUGUUACUUUCUUUGGAUCUCUGUUUAUUAAAGUGUGAC | |
| | | AAUUCAGAUGGAGUGUUUUCUGAUAAGAUUAUUCCUAAUCC | |
| | | GUGAGUUCUUAACAGUUAACAGGUGGUGGCCACUCUGUUAA | |
| | | UGUGCACACCCUGUAAGAAAGAACUAUCCUUGGACUGUUCU | |
| | | UACCAUGUGACCCCCUCAAGGGAAAGAUGGCAAACUGAUGGU | |
| | | CAGUAGAGUGACAGGUACACAUGACACUCGAGUGCUGGGUC | |
| | | UGGAGAAGCUGCAGUUAGUAUUUAGGAUAACAGUAUAUAUA | |
| | | AUAUAUAGUUCUACAUUUGGGCAGCACAGUUGGUUUCAGGC | |
| | | UAUGAAUAAAAAUCAUUGGAGUGAAACCUAAAGAAAAGUAA | |
| | | AAUUAAUAAGGAAGAGCUGCUAAAAUCAGGUUUAAGCUGAC | |
| | | ACUAUCUACAGAGCUAAAGUUUUCCAUAAUAUGUUGCUUUU | |
| | | UUUAAAAGAAAUGCAAGAAGCUGACUGAUGGAUCCCUGAGC | |
| | | UGCCAGGUAAGGAUUCCACAGGCCUGGGUUUGAUUGUGAGU | |
| | | GACCAGAAUUGUACAAUUAUUACUAAAACAGAAACAUAGUCA | |
| | | CUUCUGACUCCAUUCUUAACAUUUUUGGAUUAAAACUUGUU | |
| | | UCAGAUGACUAAUGAAAACUUCCUUUAAAACGUGAUAAAGC | |
| | | UCUAAUGUCAGCACGAUCCACAUGAUCCAUGUGCCUCCUAUA | |
| | | AAGGGAGGGGUCUGUCACCAGUGUUACGGAUUGAAUGCUAC | |
| | | GUUUAUCUUCCAACAUAGGAAGCCUGCCAGGUACUUUCUUC | |
| | | AAUAUACUAAAUUAGAAGUUUAAAAUUAAUAAGGUCUUAAU | |
| | | UAGGACCUUUGGAUGCUGAGCUUGUGGUAGUAAUUGUGUU | |
| | | CAGAAUGUUUUGAACUUAAAUUGAGAUUUAAUUUUAGUAA | |
| | | GGAAUGUGUGUCUUAGAGGCCUAGUAGUGAAGAGGAGUUU | |
| | | UCCUAGGAUUUUCCUCUCUCCUCCCUUAAUUAAGCUGCUUU | |
| | | CUGCACUAAGGGCUUCACGCAGCAACGCACUUGUUCAGUUCC | |
| | | GCACAGGGGGCUUUGCUGACUGUCUGCUCUAAAACUCUGUU | |
| | | GGCAAAACAGCUGUGGUCUCUCUUAGACUUUGAUUUUGUAG | |
| | | UAACUAGGGCAUAUAGUUGCUAUAAGCUUUCAGGAAUGGGG | |
| | | GUGGGUUACUAAUGUCUUAUGUUAUAUGAAAGUAGUUAAA | |
| | | UUUAUCCUAUAUUAAGAAGGAAGCAUUAUUGAGACUAUAUA | |
| | | CUGUUGAUUUUACUAAAAACUCUAAGAUGUCCAUUUUAACA | |
| | | GGCAAACCUAGCACAGAAAACCAGCUGGAUUUUCCCUGUGCA | |
| | | UGGUUUGAAGAGUCAGUCCUACAUAAUUGCUGACACAAUGC | |
| | | AACCUGCAACUGCCCGGAGAAAGAACAGUUCACUAAAAUUUG | |
| | | UUGUAUUUAUCAUCCAAUUCUGUUCUGUAACUGGUAACACU | |
| | | AGUUUGUCUGGCUUUAGAGAAUAGGUGAAUCUCUAAAACAG | |
| | | UAGAAACAGCUCAGUUGGGCAAGGGCCGUUCUAGUAGCAUG | |
| | | CCUGCUCCUUGGAGUUUUCCAGAUUAUUUUGUAUAGCCUAU | |
| | | UCCAGAUUCUUGUCUAAGAUUGCUUUGCUCUGUGCACUCAG | |
| | | AAUUGGUGUGCCUUUCUAUUUGUGUAAAUGAUAUAUAAUA | |
| | | CAUUGCUAGUUGUCUAGGAUCUAUUCAGGAAGUGCGUGCUG | |
| | | UGAAUUUUAAAGUAUGGGAAGAUUGUUAACAAGGGUUUCA | |
| | | AUGUUUUGUUUUUUUUUUUCUGGUAAGCUAAAAUAGAACA | |
| | | UUGUGAGGUACCUGGUUUAUUGUGGUUACAGACUUGGAAU | |
| | | AAUGUUCAUUGUUUUGUGCUAAUAAAUUGUGUUAAAAUUU | |
| | | GGUGUACAUAUGAGCAUUUUUAAAUAAAGUUAUUUUGUAG | |
| | | CACUGA | |
| mmu- | MI00006 | GGUUGCUUCAGUGAACAUUCAACGCUGUCGGUGAGUUUGGA | SEQ ID |
| miR- | 97 | AUUCAAAUAAAAACCAUCGACCGUUGAUUGUACCCUAUAGCU | NO: 531 |
| 181a | | AACC | |
| mmu- | MI00007 | AGGUCACAAUCAACAUUCAUUGCUGUCGGUGGGUUGAACUG | SEQ ID |
| miR- | 23 | UGUAGAAAAGCUCACUGAACAAUGAAUGCAACUGUGGCC | NO: 532 |
| 181b-1 | | | |
| mmu- | MI00007 | GCCAAGGGUUUGGGGGAACAUUCAACCUGUCGGUGAGUUUG | SEQ ID |
| miR- | 24 | GGCAGCUCAGACAAACCAUCGACCGUUGAGUGGACCCCGAGG | NO: 533 |
| 181c | | CCUGGA | |
| mmu- | MI00002 | ACCAUUUUUGGCAAUGGUAGAACUCACACCGGUAAGGUAAU | SEQ ID |
| miR- | 24 | GGGACCCGGUGGUUCUAGACUUGCCAACUAUGGU | NO: 534 |
| 182 | | | |
| mmu- | MI00002 | CUGUGUAUGGCACUGGUAGAAUUCACUGUGAACAGUCUCAG | SEQ ID |
| miR- | 25 | UCAGUGAAUUACCGAAGGGCCAUAAACAG | NO: 535 |
| 183 | | | |
| mmu- | MIMAT00 | AGACCUACUUAUCUACCAACAGC | SEQ ID |
| miR- | 09457 | | NO: 536 |
| 1839- | | | |
| 3p | | | |
| mmu- | MIMAT00 | AAGGUAGAUAGAACAGGUCUUG | SEQ ID |
| miR- | 09456 | | NO: 537 |
| 1839- | | | |
| 5p | | | |
| mmu- | MI00002 | CCUUUCCUUAUCACUUUUCCAGCCAGCUUUGUGACUCUAAG | SEQ ID |
| miR- | 26 | UGUUGGACGGAGAACUGAUAAGGGUAGG | NO: 538 |
| 184 | | | |
| mmu- | MI00002 | AGGGAUUGGAGAGAAAGGCAGUUCCUGAUGGUCCCCUCCCA | SEQ ID |
| miR- | 27 | GGGGCUGGCUUUCCUCUGGUCCUU | NO: 539 |
| 185 | | | |
| mmu- | MI00002 | ACUUUCCAAAGAAUUCUCCUUUUGGGCUUUCUCAUUUUAUU | SEQ ID |
| miR- | 28 | UUAAGCCCUAAGGUGAAUUUUUUGGGAAGU | NO: 540 |
| 186 | | | |
| mmu- | MIMAT00 | CAUCCCUUGCAUGGUGGAGGG | SEQ ID |
| miR- | 00217 | | NO: 541 |
| 188-5p | | | |
| mmu- | MIMAT00 | GCAAGGGAGAGGGUGAAGGGAG | SEQ ID |
| miR- | 07878 | | NO: 542 |
| 1894- | | | |
| 3p | | | |
| mmu- | MIMAT00 | CUCUCUGAUGGUGGGUGAGGAG | SEQ ID |
| miR- | 07873 | | NO: 543 |
| 1896 | | | |
| mmu- | MIMAT00 | CUUUGGAUGGAGAAAGAGGGGG | SEQ ID |
| miR- | 07864 | | NO: 544 |
| 1897- | | | |
| 5p | | | |
| mmu- | MIMAT00 | AGGUCAAGGUUCACAGGGGAUC | SEQ ID |
| miR- | 07875 | | NO: 545 |
| 1898 | | | |
| mmu- | MI00005 | UGCGUGCUUUUUGUUCUAAGGUGCAUCUAGUGCAGAUAGU | SEQ ID |
| miR- | 67 | GAAGUAGACUAGCAUCUACUGCCCUAAGUGCUCCUUCUGGCA | NO: 546 |
| 18a | | UAAGAAGUUAUGUC | |
| mmu- | MIMAT00 | CCGCUCGUACUCCCGGGGGUCC | SEQ ID |
| miR- | 07880 | | NO: 547 |
| 1901 | | | |
| mmu- | MIMAT00 | AGAGGUGCAGUAGGCAUGACUU | SEQ ID |
| miR- | 07863 | | NO: 548 |
| 1902 | | | |
| mmu- | MIMAT00 | GUUCUGCUCCUCUGGAGGGAGG | SEQ ID |
| miR- | 07874 | | NO: 549 |
| 1904 | | | |
| mmu- | MIMAT00 | CACCAGUCCCACCACGCGGUAG | SEQ ID |
| miR- | 07866 | | NO: 550 |
| 1905 | | | |
| mmu- | MI00083 | GGACAUUAGGAGCAACCUCCUAGGGUUGUUGUGAGAAUUAA | SEQ ID |
| miR- | 21 | AUGAACUGCAGCAGCCUGAGGCAGGGCUGGGCAGAGACC | NO: 551 |
| 1906-1 | | | |
| mmu- | MI00002 | AGCGGGCAACGGAAUCCCAAAAGCAGCUGUUGUCUCCAGAGC | SEQ ID |
| miR- | 33 | AUUCCAGCUGCACUUGGAUUUCGUUCCCUGCU | NO: 552 |
| 191 | | | |
| mmu- | MI00005 | CGUGCACAGGGCUCUGACCUAUGAAUUGACAGCCAGUACUCU | SEQ ID |
| miR- | 51 | UUUCUCUCCUCUGGCUGCCAAUUCCAUAGGUCACAGGUAUG | NO: 553 |
| 192 | | UUCACC | |
| mmu- | MI00002 | GAGAGCUGGGUCUUUGCGGGCAAGAUGAGAGUGUCAGUUCA | SEQ ID |
| miR- | 35 | ACUGGCCUACAAAGUCCCAGUCCUC | NO: 554 |
| 193 | | | |
| mmu- | MI00099 | AUGCCCACGGUCACCUCCAUAGUACCUGCAGCGUGCUUGACU | SEQ ID |
| miR- | 19 | UUCUCUAUGGUGCAGUUACUGUGGCUGUGGCUGUGUUCGU | NO: 555 |
| 1930 | | GC | |
| mmu- | MIMAT00 | GUUGCGGACAGCGCUAGGUCGG | SEQ ID |
| miR- | 09395 | | NO: 556 |
| 1932 | | | |
| mmu- | MIMAT00 | AUCCCGGACGAGCCCCCA | SEQ ID |
| miR- | 09414 | | NO: 557 |
| 1937b | | | |
| mmu- | MIMAT00 | AUCCCGGAAGAGCCCCCA | SEQ ID |
| miR- | 09429 | | NO: 558 |
| 1937c | | | |
| mmu- | MIMAT00 | CGGUGGGACUUGUAGUUCGGUC | SEQ ID |
| miR- | 09402 | | NO: 559 |
| 1938 | | | |
| mmu- | MIMAT00 | UCGAUUCCCUGCCAAUGCAC | SEQ ID |
| miR- | 09403 | | NO: 560 |
| 1939 | | | |
| mmu- | MI00054 | GGCCCAGAAUCGGGGUUUUGAGGGCGAGAUGAGUUUGUGU | SEQ ID |
| miR- | 84 | UUUAUCCAACUGGCCCACAAAGUCCCGCUUUUGGGGUCA | NO: 561 |
| 193b | | | |
| mmu- | MI00002 | AUCGGGUGUAACAGCAACUCCAUGUGGACUGUGCUCGGAUU | SEQ ID |
| miR- | 36 | CCAGUGGAGCUGCUGUUACUUCUGAU | NO: 562 |
| 194 | | | |
| mmu- | MIMAT00 | AUGGAGGACUGAGAAGGUGGAGCAGUU | SEQ ID |
| miR- | 09404 | | NO: 563 |
| 1940 | | | |
| mmu- | MI00099 | GGCUGGGAAGGGAGGAUCUGGGCACCUGGACCAGCUCCUCCC | SEQ ID |
| miR- | 32 | UGCAGGUGCCAGCUCCUCCCUUCCCAGUCAC | NO: 564 |
| 1943 | | | |
| mmu- | MIMAT00 | CUCUGUGCUGAAUGUCAAGUUCUGAUU | SEQ ID |
| miR- | 09409 | | NO: 565 |
| 1944 | | | |
| mmu- | MIMAT00 | AGCCGGGCAGUGGUGGCACACACUUUU | SEQ ID |
| miR- | 09412 | | NO: 566 |
| 1946a | | | |
| mmu- | MIMAT00 | CUAUACCAGGAUGUCAGCAUAGUU | SEQ ID |
| miR- | 09416 | | NO: 567 |
| 1949 | | | |
| mmu- | MI00002 | ACACCCAACUCUCCUGGCUCUAGCAGCACAGAAAUAUUGGCA | SEQ ID |
| miR- | 37 | UGGGGAAGUGAGUCUGCCAAUAUUGGCUGUGCUGCUCCAGG | NO: 568 |
| 195 | | CAGGGUGGUGA | |
| mmu- | MIMAT00 | GUAGUGGAGACUGGUGUGGCUA | SEQ ID |
| miR- | 09422 | | NO: 569 |
| 1951 | | | |
| mmu- | MIMAT00 | UGGGAAAGUUCUCAGGCUUCUG | SEQ ID |
| miR- | 09424 | | NO: 570 |
| 1953 | | | |
| mmu- | MIMAT00 | ACUGCAGAGUGAGACCCUGUU | SEQ ID |
| miR- | 09425 | | NO: 571 |
| 1954 | | | |
| mmu- | MIMAT00 | AGUCCAGGGCUGAGUCAGCGGA | SEQ ID |
| miR- | 09428 | | NO: 572 |
| 1956 | | | |
| mmu- | MIMAT00 | UAGGAAAGUGGAAGCAGUAAGU | SEQ ID |
| miR- | 09431 | | NO: 573 |
| 1958 | | | |
| mmu- | MIMAT00 | GGGGAUGUAGCUCAGUGGAG | SEQ ID |
| miR- | 09432 | | NO: 574 |
| 1959 | | | |
| mmu- | MIMAT00 | CCAGUGCUGUUAGAAGAGGGCU | SEQ ID |
| miR- | 09433 | | NO: 575 |
| 1960 | | | |
| mmu- | MIMAT00 | UGAGGUAGUAGUUAGAA | SEQ ID |
| miR- | 09434 | | NO: 576 |
| 1961 | | | |
| mmu- | MIMAT00 | AAGAUGGAGACUUUAACAUGGGU | SEQ ID |
| miR- | 09442 | | NO: 577 |
| 1969 | | | |
| mmu- | MI00005 | UGAGCCGGGACUGUUGAGUGAAGUAGGUAGUUUCAUGUUG | SEQ ID |
| miR- | 52 | UUGGGCCUGGCUUUCUGAACACAACGACAUCAAACCACCUGA | NO: 578 |
| 196a | | UUCAUGGCAGUUACUGCUUC | |
| mmu- | MI00002 | GGCUGUGCCGGGUAGAGAGGGCAGUGGGAGGUAAGAGCUCU | SEQ ID |
| miR- | 39 | UCACCCUUCACCACCUUCUCCACCCAGCAUGGCC | NO: 579 |
| 197 | | | |
| mmu- | MIMAT00 | UGUGUCACUGGGGAUAGGCUUUG | SEQ ID |
| miR- | 09444 | | NO: 580 |
| 1970 | | | |
| mmu- | MIMAT00 | GUAAAGGCUGGGCUGAGA | SEQ ID |
| miR- | 09446 | | NO: 581 |
| 1971 | | | |
| mmu- | MI00099 | GUAAAGGCUGGGCUUAGACGUGGCCUUUGGGUGUGGAAUG | SEQ ID |
| miR- | 92 | CACUUCCGUUUGUAACCGCCAUCUAACCCUGGCCUUUGACAG | NO: 582 |
| 1981 | | | |
| mmu- | MIMAT00 | UCUCACCCUAUGUUCUCCCACAG | SEQ ID |
| miR- | 09460 | | NO: 583 |
| 1982.1 | | | |
| mmu- | MIMAT00 | UCACCCUAUGUUCUCCCACAG | SEQ ID |
| miR- | 09461 | | NO: 584 |
| 1982.2 | | | |
| mmu- | MIMAT00 | ACAGUAGUCUGCACAUUGGUUA | SEQ ID |
| miR- | 00230 | | NO: 585 |
| 199a- | | | |
| 3p | | | |
| mmu- | MI00006 | GCAGCCCUCUGUUAGUUUUGCAUAGUUGCACUACAAGAAGA | SEQ ID |
| miR- | 88 | AUGUAGUUGUGCAAAUCUAUGCAAAACUGAUGGUGGCCUGC | NO: 586 |
| 19a | | | |
| mmu- | MI00007 | CACUGGUCUAUGGUUAGUUUUGCAGGUUUGCAUCCAGCUGU | SEQ ID |
| miR- | 18 | AUAAUAUUCUGCUGUGCAAAUCCAUGCAAAACUGACUGUGG | NO: 587 |
| 19b | | UGGUG | |
| mmu- | MI00005 | ACUUACGAUUAGUUUUGCAGAUUUGCAGUUCAGCGUAUAUG | SEQ ID |
| miR- | 46 | UGAAUAUAUGGCUGUGCAAAUCCAUGCAAAACUGAUUGUGG | NO: 588 |
| 19b-2 | | GA | |
| mmu- | MI00005 | CUGGGCCUCUGUGGGCAUCUUACCGGACAGUGCUGGAUUUC | SEQ ID |
| miR- | 54 | UUGGCUUGACUCUAACACUGUCUGGUAACGAUGUUCAAAGG | NO: 589 |
| 200a | | UGACCCAC | |
| mmu- | MI00002 | GCCGUGGCCAUCUUACUGGGCAGCAUUGGAUAGUGUCUGAU | SEQ ID |
| miR- | 43 | CUCUAAUACUGCCUGGUAAUGAUGACGGC | NO: 590 |
| 200b | | | |
| mmu- | MI00006 | CCCUCGUCUUACCCAGCAGUGUUUGGGUGCUGGUUGGGAGU | SEQ ID |
| miR- | 94 | CUCUAAUACUGCCGGGUAAUGAUGGAGG | NO: 591 |
| 200c | | | |
| mmu- | MI00002 | GCCUGGUCCAGUGGUUCUUGACAGUUCAACAGUUCUGUAGC | SEQ ID |
| miR- | 46 | ACAAUUGUGAAAUGUUUAGGACCACUAGACCCGGC | NO: 592 |
| 203 | | | |
| mmu- | MI00002 | UGGACUUCCCUUUGUCAUCCUAUGCCUGAGAAUAUAUGAAG | SEQ ID |
| miR- | 47 | GAGGCUGGGAAGGCAAAGGGACGUUCA | NO: 593 |
| 204 | | | |
| mmu- | MI00005 | GUGUGAUGUGACAGCUUCUGUAGCACUAAAGUGCUUAUAGU | SEQ ID |
| miR- | 68 | GCAGGUAGUGUGUAGCCAUCUACUGCAUUACGAGCACUUAA | NO: 594 |
| 20a | | AGUACUGCCAGCUGUAGAACUCCAG | |
| mmu- | MI00035 | CCUAGUAGUGCCAAAGUGCUCAUAGUGCAGGUAGUUUUUAU | SEQ ID |
| miR- | 36 | ACCACUCUACUGCAGUGUGAGCACUUCUAGUACUCCUGG | NO: 595 |
| 20b | | | |
| mmu- | MI00005 | UGUACCACCUUGUCGGAUAGCUUAUCAGACUGAUGUUGACU | SEQ ID |
| miR-21 | 69 | GUUGAAUCUCAUGGCAACAGCAGUCGAUGGGCUGUCUGACA | NO: 596 |
| | | UUUUGGUAUC | |
| mmu- | MI00006 | CCGGGGCAGUCCCUCCAGGCUCAGGACAGCCACUGCCCACCGC | SEQ ID |
| miR- | 95 | ACACUGCGUUGCUCCGGACCCACUGUGCGUGUGACAGCGGCU | NO: 597 |
| 210 | | GAUCUGUCCCUGGGCAGCGCGAACC | |
| mmu- | MI00007 | CUGCUUGGACCUGUGACCUGUGGGCUUCCCUUUGUCAUCCU | SEQ ID |
| miR- | 08 | UUGCCUAGGCCUCUGAGUGAGGCAAGGACAGCAAAGGGGGG | NO: 598 |
| 211 | | CUCAGUGGUCACCUCUACUGCAGA | |
| mmu- | MI00006 | GGGCAGCGCGCCGGCACCUUGGCUCUAGACUGCUUACUGCCC | SEQ ID |
| miR- | 96 | GGGCCGCCUUCAGUAACAGUCUCCAGUCACGGCCACCGACGC | NO: 599 |
| 212 | | CUGGCCC | |
| mmu- | MIMAT00 | GUCUUGGGAAACGGGGUGC | SEQ ID |
| miR- | 11210 | | NO: 600 |
| 2134 | | | |
| mmu- | MIMAT00 | AGAGGUCUUGGGGCCGAAAC | SEQ ID |
| miR- | 11211 | | NO: 601 |
| 2135 | | | |
| mmu- | MIMAT00 | AAGGGAACGGGCUUGGCGGAAU | SEQ ID |
| miR- | 11214 | | NO: 602 |
| 2138 | | | |
| mmu- | MIMAT00 | AGCUGCGCUGCUCCUGGUAACUGC | SEQ ID |
| miR- | 11215 | | NO: 603 |
| 2139 | | | |
| mmu- | MI00006 | GGCCUGGCUGGACAGAGUUGUCAUGUGUCUGCCUGUCUACA | SEQ ID |
| miR- | 98 | CUUGCUGUGCAGAACAUCCGCUCACCUGUACAGCAGGCACAG | NO: 604 |
| 214 | | ACAGGCAGUCACAUGACAACCCAGCCU | |
| mmu- | MIMAT00 | GUGGAGAAGGGUUCCAUGUG | SEQ ID |
| miR- | 11222 | | NO: 605 |
| 2146 | | | |
| mmu- | MI00007 | GUGAUAAUGGAGCGAGAUUUUCUGUUGUGCUUGAUCUAACC | SEQ ID |
| miR- | 00 | AUGUGCUUGCGAGGUAUGAGAAAAACAUGGUUCCGUCAAGC | NO: 606 |
| 218 | | ACCAUGGAACGUCACGCAGCUUUCUACA | |
| mmu- | MIMAT00 | ACGCCACAUUUCCCACGCCGCG | SEQ ID |
| miR- | 11286 | | NO: 607 |
| 2182 | | | |
| mmu- | MIMAT00 | UUGAACCCCUGACCUCCU | SEQ ID |
| miR- | 11287 | | NO: 608 |
| 2183 | | | |
| mmu- | MIMAT00 | CAACAGCAGUCGAUGGGCUGUC | SEQ ID |
| miR- | 04628 | | NO: 609 |
| 21a-3p | | | |
| mmu- | MI00005 | ACCUGGCUGAGCCGCAGUAGUUCUUCAGUGGCAAGCUUUAU | SEQ ID |
| miR-22 | 70 | GUCCUGACCCAGCUAAAGCUGCCAGUUGAAGAACUGUUGCCC | NO: 610 |
| | | UCUGCCCCUGGC | |
| mmu- | MI00007 | AUCCAGGUCUGGGGCAUGAACCUGGCAUACAAUGUAGAUUU | SEQ ID |
| miR- | 09 | CUGUGUUUGUUAGGCAACAGCUACAUUGUCUGCUGGGUUUC | NO: 611 |
| 221 | | AGGCUACCUGGAA | |
| mmu- | MI00007 | CCCUCAGUGGCUCAGUAGCCAGUGUAGAUCCUGUCUUUGGU | SEQ ID |
| miR- | 10 | AAUCAGCAGCUACAUCUGGCUACUGGGUCUCUGGUGGC | NO: 612 |
| 222 | | | |
| mmu- | MI00007 | UCUGGCCAUCUGCAGUGUCACGCUCCGUGUAUUUGACAAGC | SEQ ID |
| miR- | 03 | UGAGUUGGACACUCUGUGUGGUAGAGUGUCAGUUUGUCAA | NO: 613 |
| 223 | | AUACCCCAAGUGUGGCUCAUGCCUAUCAG | |
| mmu- | MGI1914 | UGGCGCCCCCGAGUGGGUGUGGUCACGUUGCCCAGCAAUGG | SEQ ID |
| miR- | 348 | GCGGUGAUUGGCCCUGGGUGGUUCAUUCGCAGCUCGUGCGU | NO: 614 |
| 22hg | | CACGACGCCGCCAGCUGAUCGGAGACUGGAGCCGGUGUGUGC | |
| | | UGGGCGCUGGGAAGAGACAGAGCGGUCGGCCGUGCGGACAG | |
| | | GUCGCAGUGAUUUUGCUCCUCUGUCCACAGCAACCCCCGCAC | |
| | | CCAGCAUCAGGAACCUGUGCCUCCCACACCCUCACCUGGCUGA | |
| | | GCCGCAGUAGUUCUUCAGUGGCAAGCUUUAUGUCCUGACCC | |
| | | AGCUAAAGCUGCCAGUUGAAGAACUGUUGCCCUCUGCCCCUG | |
| | | GCUUCGUGGAGGAAGAGGAGAAGCAGCAGCUUUGCCUAUCA | |
| | | UCCGGAAGUGUGUCCCUCCAGCACUGGGUACAUGGCUCUGC | |
| | | UGUCCUCAUCCAACAUGGAGCCUCAGAGGUGAGAAAGGGCAG | |
| | | CCUGGAAGCAACAGAGGCAGGCACAAGACAGUGGAGGACCUG | |
| | | GCCUGGAACCACAAGGGCCUAUCCGGACAUUGGUCAGAGAGG | |
| | | CACGUAGAAGCCUGGAGAACACCAGGAAAGAGAGCAGCCAGC | |
| | | CAGCCUCAGUGAAAGACACGUGCUUCCAGCCAUCUCCUCUCA | |
| | | GGACCUGCCUUCCUGGGAGAUGAAGGGCCUCCAGGAAGUAU | |
| | | GGUCCCAUCUCUACCCUGCAGUUUCUAUAAACAGCCUCAAGG | |
| | | AGCAUGAGCCACCUCUGAAAGGAAAUACACAGCAAAUUCAAA | |
| | | AAGAGAUUCAAAUGUGUAACACUGUGGGAAAACAUAUCUAU | |
| | | GACUGGGGUUGUAGCUCAGUUGGUAGGUUUGCUUAACAUG | |
| | | CACCAAGCCCUGGUUCUGUCUUCUGCAUUGCAUAAAACUGAA | |
| | | CAGGUUGGCCCAGGUCUGCAAUCCCGGCACUCUGGAGGUGG | |
| | | UGGCAAAGGAGCCUACAUUCAAGGUAAUCCUCUGCUAUACAA | |
| | | UGAGUUCUGAGCCAGCCUGGGCUAUAUGAGACUGUCUCAAA | |
| | | AAAUAAAACAAAAUAAAAUAAAGCAUUGGUUAGUAAUUCAAA | |
| | | GAAAGCAGAUGUGGCUGAAACCGUUUUCCCUGAUCAUAAUA | |
| | | CAACAAGCAAAUGAAAGCCAGAAGAAGGCUCCUGUGCCUUGU | |
| | | GUGUGGCAGUACCAACCAUUGUGAGAGAUGCCUUUGGACCU | |
| | | GGUAGUUUGCUGUCUUAGAAAUGUAUCCUAAAAUAAGGAUU | |
| | | UGGUUAUAAAAUGUUCAUCUCAGGGUUGUAAUAGAGAAAAA | |
| | | UGGAACGCAGCUGUUUGUUUGGAAGUCCAUUCCUUUUCUGC | |
| | | UGUCAUGAAAAUGUAUAGCUAGGGCUUGCCUAAGUAAAUUA | |
| | | UAUUCAUCUGAUGGUGGUGUUCUGUGCAGCCAUCCAAAGUC | |
| | | UUACAGAAGAAAAAUUGAGUGGAAAUAUAAAUAUUGAAUAC | |
| | | UAAAAAGAUUAUAAAAGUAUGAGUUUGUGACUGUUUUUAA | |
| | | AAUAUGAACACAUACUUGUAAUAUAUUUUUUUAAAAACCAU | |
| | | CCAAUUGAGUGGAAAUAUAAAUACUGAAUACUAAAAAGAUU | |
| | | AUGAAAAGUAUGAGUUUGUGACUGUUUUUAAAAUAUGAAU | |
| | | GCAUACUUGUAAUAUAUUUUUUAAAAAAACACUGAAAGUGG | |
| | | AUUCAAAAUGUUAAGAAUGGUUGUUUUUGUAUGGUGGGAU | |
| | | AGUACAAUUGUGAAUUUUCCCCUUGUUUUUUCUGUCUUUC | |
| | | UAAUUUUUAAAUAUUGUGCAUUGCUUUCAUAUGUUAAAUA | |
| | | AAAUACAAAAGACAAAUAAAUGUUUUAAAAUUUU | |
| mmu- | MI00005 | CGGACGGCUGGGGUUCCUGGGGAUGGGAUUUGAUGCCAGUC | SEQ ID |
| miR- | 71 | ACAAAUCACAUUGCCAGGGAUUUCCAACUGACCC | NO: 615 |
| 23a | | | |
| mmu- | MI00001 | GGCUGCUUGGGUUCCUGGCAUGCUGAUUUGUGACUUGAGA | SEQ ID |
| miR- | 41 | UUAAAAUCACAUUGCCAGGGAUUACCACGCAACC | NO: 616 |
| 23b | | | |
| mmu- | MI00002 | CUCCGGUGCCUACUGAGCUGAUAUCAGUUCUCAUUUCACACA | SEQ ID |
| miR-24 | 31 | CUGGCUCAGUUCAGCAGGAACAGGAG | NO: 617 |
| mmu- | MI00005 | GCCUCUCUCCGGGCUCCGCCUCCCGUGCCUACUGAGCUGAAA | SEQ ID |
| miR- | 72 | CAGUUGAUUCCAGUGCACUGGCUCAGUUCAGCAGGAACAGG | NO: 618 |
| 24-2 | | AGUCCAGCCCCCUAGGAGCUGGCA | |
| mmu- | MI00006 | GGCCAGUGUUGAGAGGCGGAGACUUGGGCAAUUGCUGGACG | SEQ ID |
| miR-25 | 89 | CUGCCCUGGGCAUUGCACUUGUCUCGGUCUGACAGUGCCGG | NO: 619 |
| | | CC | |
| mmu- | MI00005 | AAGGCCGUGGCCUCGUUCAAGUAAUCCAGGAUAGGCUGUGC | SEQ ID |
| miR- | 73 | AGGUCCCAAGGGGCCUAUUCUUGGUUACUUGCACGGGGACG | NO: 620 |
| 26a | | CGGGCCUG | |
| mmu- | MI00007 | GGCUGCGGCUGGAUUCAAGUAAUCCAGGAUAGGCUGUGUCC | SEQ ID |
| miR- | 06 | GUCCAUGAGGCCUGUUCUUGAUUACUUGUUUCUGGAGGCAG | NO: 621 |
| 26a-2 | | CG | |
| mmu- | MI00005 | UGCCCGGGACCCAGUUCAAGUAAUUCAGGAUAGGUUGUGGU | SEQ ID |
| miR- | 75 | GCUGACCAGCCUGUUCUCCAUUACUUGGCUCGGGGGCCGGU | NO: 622 |
| 26b | | GCC | |
| mmu- | MI00005 | UGGCCUGAGGAGCAGGGCUUAGCUGCUUGUGAGCAAGGUCC | SEQ ID |
| miR- | 78 | ACAGCAAAGUCGUGUUCACAGUGGCUAAGUUCCGCCCCCUGG | NO: 623 |
| 27a | | ACCC | |
| mmu- | MI00001 | AGGUGCAGAGCUUAGCUGAUUGGUGAACAGUGAUUGGUUU | SEQ ID |
| miR- | 42 | CCGCUUUGUUCACAGUGGCUAAGUUCUGCACCU | NO: 624 |
| 27b | | | |
| mmu- | MI00006 | GGUCCCUACCUUCAAGGAGCUCACAGUCUAUUGAGUUGCCU | SEQ ID |
| miR-28 | 90 | UUCUGAUUCUCCCACUAGAUUGUGAGCUGCUGGAGGGCAGG | NO: 625 |
| | | CACU | |
| mmu- | MI00003 | UUCAAUCUGUGGUACUCAAACUGUGUGACAUUUUGUUCUUU | SEQ ID |
| miR- | 91 | GUAAGAAGUGCCGCAGAGUUUGUAGUGUUGCCGAUUGAG | NO: 626 |
| 293 | | | |
| mmu- | MI00003 | UUCCAUAUAGCCAUACUCAAAAUGGAGGCCCUAUCUAAGCUU | SEQ ID |
| miR- | 92 | UUAAGUGGAAAGUGCUUCCCUUUUGUGUGUUGCCAUGUGG | NO: 627 |
| 294 | | AG | |
| mmu- | MIMAT00 | GAGGGUUGGGUGGAGGCUCUCC | SEQ ID |
| miR- | 04576 | | NO: 628 |
| 296-3p | | | |
| mmu- | MIMAT00 | AGGGCCCCCCCUCAAUCCUGU | SEQ ID |
| miR- | 00374 | | NO: 629 |
| 296-5p | | | |
| mmu- | MI00003 | AUAUGUAUGUAUGUAUGUAUGUGUGCAUGUGCAUGUGCAU | SEQ ID |
| miR- | 95 | GUAUGCAUAUUGCAUGUAUAUAUUAUGCAUACAUGU | NO: 630 |
| 297a | | | |
| mmu- | MI00005 | ACCCCUUAGAGGAUGACUGAUUUCUUUUGGUGUUCAGAGUC | SEQ ID |
| miR- | 76 | AAUAGAAUUUUCUAGCACCAUCUGAAAUCGGUUAUAAUGAU | NO: 631 |
| 29a | | UGGGGA | |
| mmu- | MI00001 | AGGAAGCUGGUUUCAUAUGGUGGUUUAGAUUUAAAUAGUG | SEQ ID |
| miR- | 43 | AUUGUCUAGCACCAUUUGAAAUCAGUGUUCU | NO: 632 |
| 29b | | | |
| mmu- | MI00005 | AUCUCUUACACAGGCUGACCGAUUUCUCCUGGUGUUCAGAG | SEQ ID |
| miR- | 77 | UCUGUUUUUGUCUAGCACCAUUUGAAAUCGGUUAUGAUGUA | NO: 633 |
| 29c | | GGGGGA | |
| mmu- | MI00004 | CCUGCUAACGGCUGCUCUGACUUUAUUGCACUACUGUACUU | SEQ ID |
| miR- | 01 | UACAGCGAGCAGUGCAAUAGUAUUGUCAAAGCAUCCGCGAGC | NO: 634 |
| 301 | | AGG | |
| mmu- | MI00041 | UUUCCUGCUGGCUGCGGGUGCUCUGACUAGGUUGCACUACU | SEQ ID |
| miR- | 22 | GUGCUGUGAGAAGCAGUGCAAUGGUAUUGUCAAAGCAUCUG | NO: 635 |
| 301b | | GGACCAGCCUCGAAG | |
| mmu- | MI00004 | CCACCACUUAAACGUGGUUGUACUUGCUUUAGACCUAAGAAA | SEQ ID |
| miR- | 02 | GUAAGUGCUUCCAUGUUUUGGUGAUGG | NO: 636 |
| 302a | | | |
| mmu- | MI00037 | CCUUUACUUUAACAUGGAGGCACUUGCUGUGCAUUUAAAAA | SEQ ID |
| miR- | 18 | UAAGUGCUUCCAUGUUUGAGUGUGG | NO: 637 |
| 302d | | | |
| mmu- | MI00140 | GGACAGAGUGUGUGUGUCUGUGUGGAGACAGGAGUUGCCCA | SEQ ID |
| miR- | 45 | CACAUGGCACUCAACUCUGCAGG | NO: 638 |
| 3082 | | | |
| mmu- | MI00001 | GCGACUGUAAACAUCCUCGACUGGAAGCUGUGAAGCCACAAA | SEQ ID |
| miR- | 44 | UGGGCUUUCAGUCGGAUGUUUGCAGCUGC | NO: 639 |
| 30a | | | |
| mmu- | MI00001 | CUAAGCCAAGUUUCAGUUCAUGUAAACAUCCUACACUCAGCU | SEQ ID |
| miR- | 45 | GUCAUACAUGCGUUGGCUGGGAUGUGGAUGUUUACGUCAGC | NO: 640 |
| 30b | | UGUCUUGGAGUAU | |
| mmu- | MI00005 | ACCAUGUUGUAGUGUGUGUAAACAUCCUACACUCUCAGCUG | SEQ ID |
| miR- | 47 | UGAGCUCAAGGUGGCUGGGAGAGGGUUGUUUACUCCUUCU | NO: 641 |
| 30c | | GCCAUGGA | |
| mmu- | MI00005 | GAGUGACAGAUAUUGUAAACAUCCUACACUCUCAGCUGUGAA | SEQ ID |
| miR- | 48 | AAGUAAGAAAGCUGGGAGAAGGCUGUUUACUCUCUCUGCCU | NO: 642 |
| 30c-2 | | U | |
| mmu- | MI00005 | AAGUCUGUGUCUGUAAACAUCCCCGACUGGAAGCUGUAAGCC | SEQ ID |
| miR- | 49 | ACAGCCAAGCUUUCAGUCAGAUGUUUGCUGCUACUGGCUC | NO: 643 |
| 30d | | | |
| mmu- | MI00002 | GGGCAGUCUUUGCUACUGUAAACAUCCUUGACUGGAAGCUG | SEQ ID |
| miR- | 59 | UAAGGUGUUGAGAGGAGCUUUCAGUCGGAUGUUUACAGCG | NO: 644 |
| 30e | | GCAGGCUGCCA | |
| mmu- | MI00005 | UGCUCCUGUAACUCGGAACUGGAGAGGAGGCAAGAUGCUGG | SEQ ID |
| miR-31 | 79 | CAUAGCUGUUGAACUGAGAACCUGCUAUGCCAACAUAUUGCC | NO: 645 |
| | | AUCUUUCCUGUCUGACAGCAGCU | |
| mmu- | MI00141 | CUAGGGGGCAGGAGCCGGAGCCCUCUGCUGAACUGACAGACG | SEQ ID |
| miR- | 01 | CUCUGCUUUGCUCCCCCAGAU | NO: 646 |
| 3104 | | | |
| mmu- | MI00007 | GCCUCGCCGCCCUCCGCCUUCUCUUCCCGGUUCUUCCCGGAG | SEQ ID |
| miR- | 04 | UCGGGAAAAGCUGGGUUGAGAGGGCGAAAAAGGAUGUGGG | NO: 647 |
| 320 | | | |
| mmu- | MI00005 | CCUCGUUGACUCCGAAGGGCUGCAGCAGCAAUUCAUGUUUU | SEQ ID |
| miR- | 90 | GGAGUAUUGCCAAGGUUCAAAACAUGAAGCGCUGCAACACCC | NO: 648 |
| 322 | | CUUCGUGGGGAA | |
| mmu- | MIMAT00 | CCACUGCCCCAGGUGCUGCU | SEQ ID |
| miR- | 00556 | | NO: 649 |
| 324-3p | | | |
| mmu- | MIMAT00 | CGCAUCCCCUAGGGCAUUGGUGU | SEQ ID |
| miR- | 00555 | | NO: 650 |
| 324-5p | | | |
| mmu- | MI00005 | CUCAUCUGUCUGUUGGGCUGGGGGCAGGGCCUUUGUGAAGG | SEQ ID |
| miR- | 98 | CGGGUUAUGCUCAGAUCGCCUCUGGGCCCUUCCUCCAGUCCC | NO: 651 |
| 326 | | GAGGCAGAUUUA | |
| mmu- | MI00006 | CUGUCUCGGAGCCUGGGGCAGGGGGGCAGGAGGGGCUCAGG | SEQ ID |
| miR- | 03 | GAGAAAGUAUCUACAGCCCCUGGCCCUCUCUGCCCUUCCGUC | NO: 652 |
| 328 | | CCCUGUCCCCAAGU | |
| mmu- | MIMAT00 | GCCCCUGGGCCUAUCCUAGAA | SEQ ID |
| miR- | 00571 | | NO: 653 |
| 331-3p | | | |
| mmu- | MIMAT00 | CUAGGUAUGGUCCCAGGGAUCC | SEQ ID |
| miR- | 04643 | | NO: 654 |
| 331-5p | | | |
| mmu- | MIMAT00 | UCAAGAGCAAUAACGAAAAAUGU | SEQ ID |
| miR- | 00766 | | NO: 655 |
| 335-5p | | | |
| mmu- | MIMAT00 | GAACGGCGUCAUGCAGGAGUU | SEQ ID |
| miR- | 04644 | | NO: 656 |
| 337-5p | | | |
| mmu- | MIMAT00 | UGAGCGCCUCGGCGACAGAGCCG | SEQ ID |
| miR- | 04649 | | NO: 657 |
| 339-3p | | | |
| mmu- | MIMAT00 | UCCCUGUCCUCCAGGAGCUCACG | SEQ ID |
| miR- | 00584 | | NO: 658 |
| 339-5p | | | |
| mmu- | MIMAT00 | UCCGUCUCAGUUACUUUAUAGC | SEQ ID |
| miR- | 00586 | | NO: 659 |
| 340-3p | | | |
| mmu- | MIMAT00 | UUAUAAAGCAAUGAGACUGAUU | SEQ ID |
| miR- | 04651 | | NO: 660 |
| 340-5p | | | |
| mmu- | MIMAT00 | UCUCACACAGAAAUCGCACCCGU | SEQ ID |
| miR- | 00590 | | NO: 661 |
| 342-3p | | | |
| mmu- | MIMAT00 | AGGGGUGCUAUCUGUGAUUGAG | SEQ ID |
| miR- | 04653 | | NO: 662 |
| 342-5p | | | |
| mmu- | MI00006 | ACCCAAGUCCAGGCCUGCUGACCCCUAGUCCAGUGCUUGUGG | SEQ ID |
| miR- | 32 | UGGCUACUGGGCCCUGAACUAGGGGUCUGGAGACCUGGGUU | NO: 663 |
| 345 | | UGAUCUCCACAGG | |
| mmu- | MIMAT00 | GCUGACCCCUAGUCCAGUGCUU | SEQ ID |
| miR- | 00595 | | NO: 664 |
| 345-5p | | | |
| mmu- | MIMAT00 | CCCUGGGAGGAGACGUGGAUUC | SEQ ID |
| miR- | 15646 | | NO: 665 |
| 3474 | | | |
| mmu- | MI00005 | CCAGCUGUGAGUAAUUCUUUGGCAGUGUCUUAGCUGGUUG | SEQ ID |
| miR- | 84 | UUGUGAGUAUUAGCUAAGGAAGCAAUCAGCAAGUAUACUGC | NO: 666 |
| 34a | | CCUAGAAGUGCUGCACAUUGU | |
| mmu- | MIMAT00 | AAUCACUAACUCCACUGCCAUC | SEQ ID |
| miR- | 04581 | | NO: 667 |
| 34b-3p | | | |
| mmu- | MIMAT00 | AGGCAGUGUAAUUAGCUGAUUGU | SEQ ID |
| miR- | 00382 | | NO: 668 |
| 34b-5p | | | |
| mmu- | MI00004 | AGUCUAGUUACUAGGCAGUGUAGUUAGCUGAUUGCUAAUAG | SEQ ID |
| miR- | 03 | UACCAAUCACUAACCACACAGCCAGGUAAAAAGACU | NO: 669 |
| 34c | | | |
| mmu- | MI00006 | AGAUGCCUUGCUCCUACAAGAGUAAAGUGCAUGCGCUUUGG | SEQ ID |
| miR- | 40 | GACAGUGAGGAAAAUAAUGUUCACAAAGCCCAUACACUUUCA | NO: 670 |
| 350 | | CCCUUUAGGAGAGUUG | |
| mmu- | MI00006 | CAUGGCACCUCCGUUUCCCUGAGGAGCCCUUUGAGCCUGGAG | SEQ ID |
| miR- | 43 | UGAAAAAAAAAAACAGGUCAAGAGGCGCCUGGGAACUGGAGA | NO: 671 |
| 351 | | AGAGUGUUAAACUUC | |
| mmu- | MI00007 | GAAGCUUAUCAGAAUCUCCAGGGGUACUUAGUAUUUGAAAA | SEQ ID |
| miR- | 61 | GUCCCCCAGGUGUGAUUCUGAUUUGUUUC | NO: 672 |
| 361 | | | |
| mmu- | MIMAT00 | AACACACCUGUUCAAGGAUUCA | SEQ ID |
| miR- | 04684 | | NO: 673 |
| 362-3p | | | |
| mmu- | MIMAT00 | AAUCCUUGGAACCUAGGUGUGAAU | SEQ ID |
| miR- | 00706 | | NO: 674 |
| 362-5p | | | |
| mmu- | MI00007 | UGUUAUCAGGUGGAACACGAUGCAAUUUUGGUUGGUGUAA | SEQ ID |
| miR- | 65 | UAGGAGGAAAAUUGCACGGUAUCCAUCUGUAAACC | NO: 675 |
| 363 | | | |
| mmu- | MI00007 | ACCGCAGGGAAAAUGAGGGACUUUUGGGGGCAGAUGUGUUU | SEQ ID |
| miR- | 68 | CCAUUCCGCUAUCAUAAUGCCCCUAAAAAUCCUUAUUGCUCU | NO: 676 |
| 365 | | UGCA | |
| mmu- | MIMAT00 | AUAUAAUACAACCUGCUAAGUG | SEQ ID |
| miR- | 03727 | | NO: 677 |
| 374b- | | | |
| 5p | | | |
| mmu- | MI00007 | CCCCGCGACGAGCCCCUCGCACAAACCGGACCUGAGCGUUUU | SEQ ID |
| miR- | 92 | GUUCGUUCGGCUCGCGUGAGGC | NO: 678 |
| 375 | | | |
| mmu- | MI00011 | UGGUAUUUAAAAGGUGGAUAUUCCUUCUAUGGUUACGUGC | SEQ ID |
| miR- | 62 | UUCCUGGAUAAUCAUAGAGGAACAUCCACUUUUUCAGUAUC | NO: 679 |
| 376b | | A | |
| mmu- | MI00035 | UUUGGUAUUUAAAAGGUGGAUAUUCCUUCUAUGUUUAUGC | SEQ ID |
| miR- | 33 | UUUUUGUGAUUAAACAUAGAGGAAAUUUCACGUUUUCAGU | NO: 680 |
| 376c | | GUCAAA | |
| mmu- | MI00007 | UACUUAAAGCGAGGUUGCCCUUUGUAUAUUCGGUUUAUUGA | SEQ ID |
| miR- | 98 | CAUGGAAUAUACAAGGGCAAGCUCUCUGUGAGUA | NO: 681 |
| 381 | | | |
| mmu- | MIMAT00 | GAAUGUUGCUCGGUGAACCCCU | SEQ ID |
| miR- | 01090 | | NO: 682 |
| 409-3p | | | |
| mmu- | MI00011 | GGGUACUUGAGGAGAGGUUGUCUGUGAUGAGUUCGCUUUA | SEQ ID |
| miR- | 61 | UUAAUGACGAAUAUAACACAGAUGGCCUGUUUUCAAUACCA | NO: 683 |
| 410 | | | |
| mmu- | MI00011 | UGGUACUUGGAGAGAUAGUAGACCGUAUAGCGUACGCUUUA | SEQ ID |
| miR- | 63 | UCUGUGACGUAUGUAACACGGUCCACUAACCCUCAGUAUCA | NO: 684 |
| 411 | | | |
| mmu- | MIMAT00 | UGAGGGGCAGAGAGCGAGACUUU | SEQ ID |
| miR- | 04825 | | NO: 685 |
| 423-5p | | | |
| mmu- | MI00014 | AAAGUGCUUUGGAAUGACACGAUCACUCCCGUUGAGUGGGC | SEQ ID |
| miR- | 47 | ACCCAAGAAGCCAUCGGGAAUGUCGUGUCCGCCCAGUGCUCU | NO: 686 |
| 425 | | UU | |
| mmu- | MIMAT00 | UUUGAACCAUCACUCGACUCCU | SEQ ID |
| miR- | 01422 | | NO: 687 |
| 434-3p | | | |
| mmu- | MIMAT00 | AGGCAGUGUUGUUAGCUGGC | SEQ ID |
| miR- | 05447 | | NO: 688 |
| 449b | | | |
| mmu- | MIMAT00 | AUUGGGAACAUUUUGCAUGCAU | SEQ ID |
| miR- | 03512 | | NO: 689 |
| 450b- | | | |
| 3p | | | |
| mmu- | MIMAT00 | AAACCGUUACCAUUACUGAGUU | SEQ ID |
| miR- | 01632 | | NO: 690 |
| 451 | | | |
| mmu- | MI00023 | CUUUACCUAAUUUGUUGUCCAUCAUGUAAAACAUAAAUGAU | SEQ ID |
| miR- | 98 | GAUAGACACCAUAUAAGGUAGAGGAAGGUUCACU | NO: 691 |
| 463 | | | |
| mmu- | MIMAT00 | UAUUUAGAAUGGCACUGAUGUGA | SEQ ID |
| miR- | 02106 | | NO: 692 |
| 465a- | | | |
| 5p | | | |
| mmu- | MIMAT00 | UAUUUAGAAUGGCGCUGAUCUG | SEQ ID |
| miR- | 04873 | | NO: 693 |
| 465c- | | | |
| 5p | | | |
| mmu- | MI00055 | UAUAUGUGUUGAUGUGUGUGUACAUGUACAUGUGUGAAUA | SEQ ID |
| miR- | 04 | UGAUAUACAAAUACAUACACGCACACAUAAGACACAUAUGA | NO: 694 |
| 466b-3 | | | |
| mmu- | MIMAT00 | GAUGUGUGUGUACAUGUACAUA | SEQ ID |
| miR- | 04879 | | NO: 695 |
| 466e- | | | |
| 5p | | | |
| mmu- | MIMAT00 | AUACAGACACAUGCACACACA | SEQ ID |
| miR- | 04883 | | NO: 696 |
| 466g | | | |
| mmu- | MIMAT00 | UGUGUGCAUGUGCAUGUGUGUAA | SEQ ID |
| miR- | 05848 | | NO: 697 |
| 466j | | | |
| mmu- | MIMAT00 | UGUGUGUGUACAUGUACAUGUGA | SEQ ID |
| miR- | 05845 | | NO: 698 |
| 466k | | | |
| mmu- | MI00024 | CUGUGUGCGUAAGUGCCUGCAUGUAUAUGCGUGUAUAUUU | SEQ ID |
| miR- | 02 | UAUGCAUAUACAUACACACACCUACACACACAU | NO: 699 |
| 467a | | | |
| mmu- | MI00046 | CCGUGUGCGUAAGUGCCUGCAUGUAUAUGCGUGUAUAUUUU | SEQ ID |
| miR- | 71 | AUGCAUAUACAUACACACACCAACACACACAU | NO: 700 |
| 467b | | | |
| mmu- | MI00055 | CCUUUGUGCAUAAGUGCGUGCAUGUAUAUGUGUGUAUAUU | SEQ ID |
| miR- | 12 | UUAUGCAUAUACAUACACACACCUAUACACACAUGCACACAG | NO: 701 |
| 467c | | ACAUGCGAGAAUGGC | |
| mmu- | MI00055 | CCUGUGUGCAUAAGUGCGCGCAUGUAUAUGCGUGUAUAUUU | SEQ ID |
| miR- | 13 | UAUGCAUAUACAUACACACACCUACACACACAUGCACACAGAC | NO: 702 |
| 467d | | A | |
| mmu- | MIMAT00 | AUAUACACACACACACCUACA | SEQ ID |
| miR- | 05846 | | NO: 703 |
| 467f | | | |
| mmu- | MIMAT00 | AUAAGUGUGUGCAUGUAUAUGU | SEQ ID |
| miR- | 05855 | | NO: 704 |
| 467h | | | |
| mmu- | MI00024 | CAGUGCUCUUCUUGGACUGGCACUGGUGAGUUAAACUAAAU | SEQ ID |
| miR- | 05 | ACAACCAGUACCUUUCUGAGAAGAGUAAAGCUCA | NO: 705 |
| 470 | | | |
| mmu- | MIMAT00 | UCAGGCUCAGUCCCCUCCCGAU | SEQ ID |
| miR- | 03127 | | NO: 706 |
| 484 | | | |
| mmu- | MIMAT00 | AGUCAUACACGGCUCUCCUCUC | SEQ ID |
| miR- | 03129 | | NO: 707 |
| 485-3p | | | |
| mmu- | MI00034 | CAGCCAGCUCUGAUCUCGCCCUCCCUGAGGGGUCCUGUACUG | SEQ ID |
| miR- | 93 | AGCUGCCCCGAGGUCCUUCACUGUGCUCAGCUCGGGGCAGCU | NO: 708 |
| 486 | | CAGUACAGGAUGCGUCAGGGUGGGAGACAACGGGGAACAAG | |
| | | CCA | |
| mmu- | MI00035 | UGGUACUUGGAGAGUGGUUAUCCCUGUCCUCUUCGCUUCAC | SEQ ID |
| miR- | 34 | UCAUGCCGAAUCGUACAGGGUCAUCCACUUUUUCAGUAUCA | NO: 709 |
| 487b | | | |
| mmu- | MI00034 | ACUGCUGCAGUGGCAGCUUGGUUGUCAUAUGUGUGAUGACA | SEQ ID |
| miR- | 76 | CUUUCUAAAGUCUUCCAGAAUGACACCACAUAUAUGGCAGCU | NO: 710 |
| 489 | | AAACUGUUACAUGGAACAACAAGU | |
| mmu- | MI00050 | UGGAAAGUUCAUUGUUCGACACCAUGGAUCUCCAGGUGGGU | SEQ ID |
| miR- | 02 | CACGUUUAGAUAUACACCAACCUGGAGGACUCCAUGCUGUUG | NO: 711 |
| 490 | | A | |
| mmu- | MI00035 | UUGAUACUUGAAGGAGAGGUUGUCCGUGUUGUCUUCUCUU | SEQ ID |
| miR- | 32 | UAUUUAUGAUGAAACAUACACGGGAAACCUCUUUUUUAGUA | NO: 712 |
| 494 | | UCAA | |
| mmu- | MI00046 | AAAGAAGUUGCCCAUGUUAUUUUUCGCUUUUAUUUGUGACG | SEQ ID |
| miR- | 39 | AAACAAACAUGGUGCACUUCUU | NO: 713 |
| 495 | | | |
| mmu- | MI00045 | AGUGUUCGAAUGGAGGUUGCCCAUGGUGUGUUCAUUUUAU | SEQ ID |
| miR- | 89 | UUAUGAUGAGUAUUACAUGGCCAAUCUCCUUUCGGCACU | NO: 714 |
| 496 | | | |
| mmu- | MI00046 | CCUGCCCCCGCCCCAGCAGCACACUGUGGUUUGUACGGCACU | SEQ ID |
| miR- | 36 | GUGGCCACGUCCAAACCACACUGUGGUGUUAGAGCGAGGGU | NO: 715 |
| 497 | | A | |
| mmu- | MI00047 | CUCCUCUGCUCCCCCUCUCUAAUCCUUGCUAUCUGGGUGCUU | SEQ ID |
| miR- | 02 | AGUGCUAUCUCAAUGCAAUGCACCUGGGCAAGGGUUCAGAG | NO: 716 |
| 500 | | AAGGUGAGC | |
| mmu- | MIMAT00 | AAUGCACCCGGGCAAGGAUUUG | SEQ ID |
| miR- | 03509 | | NO: 717 |
| 501-3p | | | |
| mmu- | MI00035 | UGCCCUAGCAGCGGGAACAGUACUGCAGUGAGUGUUUGGUG | SEQ ID |
| miR- | 38 | CCCUGGAGUAUUGUUUCCACUGCCUGGGUA | NO: 718 |
| 503 | | | |
| mmu- | MIMAT00 | UGAUUGACAUUUCUGUAAUGG | SEQ ID |
| miR- | 04891 | | NO: 719 |
| 509-3p | | | |
| mmu- | MIMAT00 | CGGCGCCCCACGGAGCCCCGAGC | SEQ ID |
| miR- | 20642 | | NO: 720 |
| 5131 | | | |
| mmu- | MI00032 | CAGAUUUGCUUUUUCUCUUCCAUGCCUUGAGUGUAGGACCG | SEQ ID |
| miR- | 06 | UUGACAUCUUAAUUACCCUCCCACACCCAAGGCUUGCAGGAG | NO: 721 |
| 532 | | AGCAAGCCUUCUC | |
| mmu- | MIMAT00 | CCUCCCACACCCAAGGCUUGCA | SEQ ID |
| miR- | 04781 | | NO: 722 |
| 532-3p | | | |
| mmu- | MIMAT00 | CAUGCCUUGAGUGUAGGACCGU | SEQ ID |
| miR- | 02889 | | NO: 723 |
| 532-5p | | | |
| mmu- | MI00035 | UACUUGAGGAGAAAUUAUCCUUGGUGUGUUGGCUCUUUUG | SEQ ID |
| miR- | 20 | GAUGAAUCAUACAAGGAUAAUUUCUUUUUGAGUA | NO: 724 |
| 539 | | | |
| mmu- | MIMAT00 | UGUGACAGAUUGAUAACUGAAA | SEQ ID |
| miR- | 03172 | | NO: 725 |
| 542-3p | | | |
| mmu- | MIMAT00 | AUGGUGGCACGGAGUC | SEQ ID |
| miR- | 03166 | | NO: 726 |
| 546 | | | |
| mmu- | MI00055 | UGCGGGCGUGUGAGUGUGUGUGUGUGAGUGUGUGUCGCUC | SEQ ID |
| miR- | 18 | CAAGUCCACGCUCAUGCACACACCCACACGCCCGCACG | NO: 727 |
| 574 | | | |
| mmu- | MIMAT00 | CACGCUCAUGCACACACCCACA | SEQ ID |
| miR- | 04894 | | NO: 728 |
| 574-3p | | | |
| mmu- | MIMAT00 | UCCGAGCCUGGGUCUCCCUCUU | SEQ ID |
| miR- | 03783 | | NO: 729 |
| 615-3p | | | |
| mmu- | MIMAT00 | GGGGGUCCCCGGUGCUCGGAUC | SEQ ID |
| miR- | 04837 | | NO: 730 |
| 615-5p | | | |
| mmu- | MI00049 | AGGAACAGCUAUGUACUGCACAACCCUAGGAGGGGGUGCCAU | SEQ ID |
| miR- | 65 | UCACAUAGAGUAUAAUUGAAUGGCGCCACUAGGGUUGUGCA | NO: 731 |
| 652 | | GUGUACAGCCUACAC | |
| mmu- | MIMAT00 | UGGUAAGCUGCAGAACAUGUGU | SEQ ID |
| miR- | 04897 | | NO: 732 |
| 654-5p | | | |
| mmu- | MI00041 | AGAACAGGGUCUCCUUGAGGGGCCUCUGCCUCUAUCCAGGA | SEQ ID |
| miR- | 71 | UUAUGUUUUUAUGACCAGGAGGCUGAGGUCCCUUACAGGCG | NO: 733 |
| 665 | | GCCUCUUACUCU | |
| mmu- | MIMAT00 | GGCUGCAGCGUGAUCGCCUGCU | SEQ ID |
| miR- | 04823 | | NO: 734 |
| 666-3p | | | |
| mmu- | MI00041 | GUGGGUACUGGCCUCGGUGCUGGUGGAGCAGUGAGCACGCC | SEQ ID |
| miR- | 96 | AUACAUUAUAUCUGUGACACCUGCCACCCAGCCCAAGGCCCCU | NO: 735 |
| 667 | | AGGCCCAC | |
| mmu- | MI00041 | GGUAAGUGUGCCUCGGGUGAGCAUGCACUUAAUGUAGGUGU | SEQ ID |
| miR- | 34 | AUGUCACUCGGCUCGGCCCACUACC | NO: 736 |
| 668 | | | |
| mmu- | MI00062 | CAGCCCGGGAUUUGUGUGUUGCUUGCUCUAUAUGUGUGUA | SEQ ID |
| miR- | 81 | UACUUGUGUGUGCAUGUAUAUGUGUGUAUAUGAAUAUACA | NO: 737 |
| 669d | | UAUACAUACACACCCAUAUACACACGCAUGCAUGCACACAC | |
| mmu- | MI00099 | GUUUGUGCAUGUGCGUAUAGUUGUGUGUGCAUGUAUAUGU | SEQ ID |
| miR- | 42 | GUGUAUAUGAAUAUACAUAUACAUACACACCCAUAUAUACAC | NO: 738 |
| 669l | | GCAUUCAUAUGAACAC | |
| mmu- | MIMAT00 | AUUUGUGUGUGGAUGUGUGU | SEQ ID |
| miR- | 09427 | | NO: 739 |
| 669n | | | |
| mmu- | MI00042 | GGUUUGGAGGUGGGCCUGACAUCCCUGAGUGUAUGUGGUG | SEQ ID |
| miR- | 95 | AACCUGAACUUGCCCUGGGUUUCCUCAUAUCCAUUCAGGAGU | NO: 740 |
| 670 | | GUCAGCUGCCUCUUCGCU | |
| mmu- | MIMAT00 | UCCGGUUCUCAGGGCUCCACC | SEQ ID |
| miR- | 04821 | | NO: 741 |
| 671-3p | | | |
| mmu- | MI00042 | GAUGGUGAUCUAGCCCUUUAGUUUUGAGGUUGGUGUACUG | SEQ ID |
| miR- | 58 | UGUGUGAGUAUACAUAUUUAUCACACACAGUCACUAUCUUC | NO: 742 |
| 672 | | GAAAGUGAGGGUGCACAUC | |
| mmu- | MI00046 | UGGAGCCUGAGGGGCUCACAGCUCUGGUCCUUGGAGCUCCA | SEQ ID |
| miR- | 01 | GAGAAAAUGUUGCUCCGGGGCUGAGUUCUGUGCACCCCCCU | NO: 743 |
| 673 | | UGCCCUCCA | |
| mmu- | MIMAT00 | UCCGGGGCUGAGUUCUGUGCACC | SEQ ID |
| miR- | 04824 | | NO: 744 |
| 673-3p | | | |
| mmu- | MI00046 | GGCCUAGUCAUCACCCUGAGCCUUGCACUGAGAUGGGAGUG | SEQ ID |
| miR- | 11 | GUGUAAGGCUCAGGUAUGCACAGCUCCCAUCUCAGAACAAGG | NO: 745 |
| 674 | | CUCGGGUGUGCUCAGCU | |
| mmu- | MIMAT00 | CUGUAUGCCCUAACCGCUCAGU | SEQ ID |
| miR- | 03726 | | NO: 746 |
| 675-3p | | | |
| mmu- | MIMAT00 | GGGCAUCUGCUGACAUGGGGG | SEQ ID |
| miR- | 03457 | | NO: 747 |
| 680 | | | |
| mmu- | MIMAT00 | CUGCAGUCACAGUGAAGUCUG | SEQ ID |
| miR- | 03459 | | NO: 748 |
| 682 | | | |
| mmu- | MIMAT00 | AGUUUUCCCUUCAAGUCAA | SEQ ID |
| miR- | 03462 | | NO: 749 |
| 684 | | | |
| mmu- | MIMAT00 | UCAAUGGCUGAGGUGAGGCAC | SEQ ID |
| miR- | 03463 | | NO: 750 |
| 685 | | | |
| mmu- | MIMAT00 | CUAUCCUGGAAUGCAGCAAUGA | SEQ ID |
| miR- | 03466 | | NO: 751 |
| 687 | | | |
| mmu- | MIMAT00 | AAAGGCUAGGCUCACAACCAAA | SEQ ID |
| miR- | 03469 | | NO: 752 |
| 690 | | | |
| mmu- | MIMAT00 | AUCUCUUUGAGCGCCUCACUC | SEQ ID |
| miR- | 03471 | | NO: 753 |
| 692 | | | |
| mmu- | MIMAT00 | CUGAAAAUGUUGCCUGAAG | SEQ ID |
| miR- | 03474 | | NO: 754 |
| 694 | | | |
| mmu- | MIMAT00 | GCGUGUGCUUGCUGUGGG | SEQ ID |
| miR- | 03483 | | NO: 755 |
| 696 | | | |
| mmu- | MI00046 | UCAUCUCUGCCCUCACUGUAAGGGAGGGUGGUGGCUAUGUG | SEQ ID |
| miR- | 82 | GGUGGGACAGGGAUGUUCAGUUGCUAAAGACAUUCUCGUUU | NO: 756 |
| 698 | | CCUUCCCUCAGUGUCCCCAGAUGGUGA | |
| mmu- | MI00046 | UUCACUGGGAGUAAGGCUCCUUCCUGUGCUUGCAGGGGAGA | SEQ ID |
| miR- | 84 | AAUACGAACUGCACGCGGGAACCGAGUCCACCCCCAGU | NO: 757 |
| 700 | | | |
| mmu- | MI00046 | CGGGACAAGGUGAGUGGGGUGGUUGGCAUGGGUUGCCCAU | SEQ ID |
| miR- | 86 | GGGGACUCGACGCUGUGCCCACAGCCUCCUGAUGUCCUCCUC | NO: 758 |
| 702 | | ACGCAUGCCCACCCUUUACCCCGCUCC | |
| mmu- | MIMAT00 | AGACAUGUGCUCUGCUCCUAG | SEQ ID |
| miR- | 03494 | | NO: 759 |
| 704 | | | |
| mmu- | MIMAT00 | AGAGAAACCCUGUCUCAAAAAA | SEQ ID |
| miR- | 03496 | | NO: 760 |
| 706 | | | |
| mmu- | MI00046 | CUGUGUUUGAAAUGGGGACUGCCCUCAAGGAGCUUACAAUC | SEQ ID |
| miR- | 92 | UAGCUGGGGGUAGAUGACUUGCACUUGAACACAACUAGACU | NO: 761 |
| 708 | | GUGAGCUUCUAGAGGGCAGGGGCCUUA | |
| mmu- | MIMAT00 | CCAAGUCUUGGGGAGAGUUGAG | SEQ ID |
| miR- | 03500 | | NO: 762 |
| 710 | | | |
| mmu- | MI00046 | UCUCCGCUUCUCCUUCACCCGGGCGGUACCCGCUCCGGCGCC | SEQ ID |
| miR- | 96 | GGCCCGCGGGACGCCGCGGCGUCCGUGCGCCGAUGCGAGUCA | NO: 763 |
| 712 | | CCCCCGGGUGUUGCGAGUUCGGGGA | |
| mmu- | MIMAT00 | AUCUCGGCUACAGAAAAAUGUU | SEQ ID |
| miR- | 03465 | | NO: 764 |
| 719 | | | |
| mmu- | MIMAT00 | AUCUCGCUGGGGCCUCCA | SEQ ID |
| miR- | 03484 | | NO: 765 |
| 720 | | | |
| mmu- | MIMAT00 | CAGUGCAAUUAAAAGGGGGAA | SEQ ID |
| miR- | 03515 | | NO: 766 |
| 721 | | | |
| mmu- | MI00052 | UUGAUCUACGUAGAUUGGUACCUAUCAUGUAAAUCAUGUAA | SEQ ID |
| miR- | 05 | GCAUGAGAGAUGCCAUUCUAUGUAGAUUAA | NO: 767 |
| 741 | | | |
| mmu- | M100052 | CUGUAUUCAGAUUGGUGCCUGUCAUGUUUAUAAGAAUGAAA | SEQ ID |
| miR- | 07 | GACACCAAGCUGAGUAGAGUA | NO: 768 |
| 743a | | | |
| mmu- | MI00041 | GGCUGGGCAAGGUGCGGGGCUAGGGCUAACAGCAGUCUUAC | SEQ ID |
| miR- | 24 | UGACGGUUUCCUGGAAACCACACACAUGCUGUUGCCACUAAC | NO: 769 |
| 744 | | CUCAACCUUACUCGGUC | |
| mmu- | MIMAT00 | GCAGCAGGGUGAAACUGACACA | SEQ ID |
| miR- | 03893 | | NO: 770 |
| 761 | | | |
| mmu- | MIMAT00 | CCAGCUGGGAAGAACCAGUGGC | SEQ ID |
| miR- | 03896 | | NO: 771 |
| 763 | | | |
| mmu- | MI00042 | GCCACCUUCUGUGCCCCCAGCACCACGUGUCUGGGCCACGUG | SEQ ID |
| miR- | 03 | AGCAACGCCACGUGGGCCUGACGUGGAGCUGGGGCCGCAGGG | NO: 772 |
| 770 | | GUCUGAUGGC | |
| mmu- | MI00007 | UUGGAUGUUGGCCUAGUUCUGUGUGGAAGACUAGUGAUUU | SEQ ID |
| miR-7a | 28 | UGUUGUUUUUAGAUAACUAAAACGACAACAAAUCACAGUCU | NO: 773 |
| | | GCCAUAUGGCACAGGCCACCUCUACAG | |
| mmu- | MI00007 | AGGAGCGGAGUACGUGAGCCAGUGCUAUGUGGAAGACUUGU | SEQ ID |
| miR-7b | 30 | GAUUUUGUUGUUCUGAUAUGAUAUGACAACAAGUCACAGCC | NO: 774 |
| | | AGCCUCAUAGCGUGGACUCCUAUCACCUU | |
| mmu- | MIMAT00 | UGUGAGUUGUUCCUCACCUGGA | SEQ ID |
| miR- | 04210 | | NO: 775 |
| 804 | | | |
| mmu- | MIMAT00 | GAAUUGAUCAGGACAUAGGG | SEQ ID |
| miR- | 04211 | | NO: 776 |
| 805 | | | |
| mmu- | MI00055 | AACUUGUUAGAAGGUUACUUGUUAGUUCAGGACCUCAUUAC | SEQ ID |
| miR- | 49 | UUUCUGCCUGAACUAUUGCAGUAGCCUCCUAACUGGUUAU | NO: 777 |
| 872 | | | |
| mmu- | MI00054 | UUAGCCCUGCGGCCCCACGCACCAGGGUAAGAGAGACUCACU | SEQ ID |
| miR- | 79 | UCCUGCCCUGGCCCGAGGGACCGACUGGCUGGGC | NO: 778 |
| 874 | | | |
| mmu- | MIMAT00 | CCUGAAAAUACUGAGGCUAUG | SEQ ID |
| miR- | 04938 | | NO: 779 |
| 875-3p | | | |
| mmu- | MI00055 | GUAGAGGAGAUGGCGCAGGGGACACAAGGUAGGCCUUGCGG | SEQ ID |
| miR- | 53 | GUCUGUGGACCCUUGGACAUGUGUCCUCUUCUCCCUCCUCCC | NO: 780 |
| 877 | | AG | |
| mmu- | MIMAT00 | GCAUGACACCACACUGGGUAGA | SEQ ID |
| miR- | 04933 | | NO: 781 |
| 878-3p | | | |
| mmu- | MI00054 | UGCACUGCAAUACUCAGAUUGAUAUGAGUCACUUCCUAUUG | SEQ ID |
| miR- | 73 | CAUGUUACUCCAUCCUCUCUGAGUAGAGUAAGGCACA | NO: 782 |
| 880 | | | |
| mmu- | MI00054 | UGCAGUACAAUAUUCAGAGAGAUAACAGUCACAUCUUUUCU | SEQ ID |
| miR- | 74 | AAAGUAACUGUGUCUUUUCUGAAUAGAGUAAUGUUCA | NO: 783 |
| 881 | | | |
| mmu- | MIMAT00 | UAACUGCAACAGCUCUCAGUAU | SEQ ID |
| miR- | 04849 | | NO: 784 |
| 883a- | | | |
| 3p | | | |
| mmu- | MI00007 | CGGGGUUGGUUGUUAUCUUUGGUUAUCUAGCUGUAUGAGU | SEQ ID |
| miR-9 | 20 | GGUGUGGAGUCUUCAUAAAGCUAGAUAACCGAAAGUAAAAA | NO: 785 |
| | | UAACCCCA | |
| mmu- | MI00007 | CUUUCUACACAGGUUGGGAUUUGUCGCAAUGCUGUGUUUCU | SEQ ID |
| miR- | 19 | CUGUAUGGUAUUGCACUUGUCCCGGCCUGUUGAGUUUGG | NO: 786 |
| 92a | | | |
| mmu- | MIMAT00 | AGGUGGGGAUUGGUGGCAUUAC | SEQ ID |
| miR- | 04635 | | NO: 787 |
| 92a-2 | | | |
| mmu- | MI00005 | AGUCAUGGGGGCUCCAAAGUGCUGUUCGUGCAGGUAGUGUA | SEQ ID |
| miR-93 | 81 | AUUACCUGACCUACUGCUGAGCUAGCACUUCCCGAGCCCCCA | NO: 788 |
| | | GGACA | |
| mmu- | MI00005 | CUGCACAUGCUGGGGUGAGGUAGUAAGUUGUAUUGUUGUG | SEQ ID |
| miR-98 | 86 | GGGUAGGGAUUUUAGGCCCCAGUAAGAAGAUAACUAUACAA | NO: 789 |
| | | CUUACUACUUUCCUUGGUGUGUGGCAU | |
| mmu- | MI00001 | CAUAAACCCGUAGAUCCGAUCUUGUGGUGAAGUGGACCGCGC | SEQ ID |
| miR- | 46 | AAGCUCGUUUCUAUGGGUCUGUG | NO: 790 |
| 99a | | | |
| mmu- | MI00001 | GGCACCCACCCGUAGAACCGACCUUGCGGGGCCUUCGCCGCAC | SEQ ID |
| miR- | 47 | ACAAGCUCGUGUCUGUGGGUCCGUGUC | NO: 791 |
| 99b | | | |
| rno- | MI00034 | UGCCUACUCAGAGCACAUACUUCUUUAUGUACCCAUAUGAAC | SEQ ID |
| miR-1 | 89 | AUAGAAUGCUAUGGAAUGUAAAGAAGUGUGUAUUUUGGGU | NO: 792 |
| | | AGGUA | |
| rno- | MI00008 | UGCGCUCCCCUCAGUCCCUGAGACCCUAACUUGUGAUGUUUA | SEQ ID |
| miR- | 96 | CCGUUUAAAUCCACGGGUUAGGCUCUUGGGAGCUGCGAGUC | NO: 793 |
| 125b | | GUGC | |
| rno- | MI00063 | UGGCCACCUGGCCCUGAGAACUGAAUUCCAUAGGCUGUGAAC | SEQ ID |
| miR- | 42 | UCUAGCAGAUGCCCUAGGGACUCAGUUCUGGUGCCUGGCUG | NO: 794 |
| 146b | | UGCUA | |
| rno- | MIMAT00 | GAAGUUCUGUUAUACACUCAGG | SEQ ID |
| miR- | 04645 | | NO: 795 |
| 148b- | | | |
| 5p | | | |
| rno- | MI00061 | UGCUUCUGUGUGAUAUGUUUGAUAUUAGGUUGUUAAAUUA | SEQ ID |
| miR- | 35 | UGAACCAACUAAAUGUCAAACAUUCUCACAGCAGUGAG | NO: 796 |
| 190b | | | |
| rno- | MI00009 | GGCUACAGCCCUUCUUCAUGUGACUCGUGGACUUCCCUUUG | SEQ ID |
| miR- | 46 | UCAUCCUAUGCCUGAGAAUAUAUGAAGGAGGCUGGGAAGGC | NO: 797 |
| 204 | | AAAGGGACGUUCAAUUGUCAUCACUGGC | |
| rno- | MIMAT00 | CUUCUCCUGGCUCUCCUCCCUUU | SEQ ID |
| miR- | 03115 | | NO: 798 |
| 207 | | | |
| rno- | MI00035 | GUAGUGCCAAAGUGCUCAUAGUGCAGGUAGGUUUUGCUGCA | SEQ ID |
| miR- | 54 | CUCUACUGCAGUGUGAGCACUUCUGGUACUC | NO: 799 |
| 20b | | | |
| rno- | MIMAT00 | AGAGUUGCGUCUGGACGUCCCG | SEQ ID |
| miR- | 04741 | | NO: 800 |
| 219a-1- | | | |
| 3p | | | |
| rno- | MI00034 | GGGCUUUCAAGUCACUAGUGGUUCCGUUUAGUAGAUGGUU | SEQ ID |
| miR- | 83 | UUUGCAUUGUUUCAAAAUGGUGCCCUAGUGACUACAAAGCC | NO: 801 |
| 224 | | C | |
| rno- | MIMAT00 | AGGGCUUAGCUGCUUGUGAGCA | SEQ ID |
| miR- | 04715 | | NO: 802 |
| 27a-5p | | | |
| rno- | MI00008 | CUUCAGGAAGCUGGUUUCAUAUGGUGGUUUAGAUUUAAAU | SEQ ID |
| miR- | 64 | AGUGAUUGUCUAGCACCAUUUGAAAUCAGUGUUCUUGGUGG | NO: 803 |
| 29b-1 | | | |
| rno- | MI00008 | AUCUCUUACACAGGCUGACCGAUUUCUCCUGGUGUUCAGAG | SEQ ID |
| miR- | 65 | UCUGUUUUUGUCUAGCACCAUUUGAAAUCGGUUAUGAUGUA | NO: 804 |
| 29c | | GGGGGA | |
| rno- | MIMAT00 | CCUUGAGGGGCAUGAGGGU | SEQ ID |
| miR- | 00561 | | NO: 805 |
| 327 | | | |
| rno- | MI00006 | AUGUGACCGUGCCUCUCACCCUUCCAUAUCUAGUCUCUGAGA | SEQ ID |
| miR- | 13 | AAAAUGAAGACUGGAUUCCAUGAAGGGAUGUGAGGCCUGGA | NO: 806 |
| 336 | | AACUGGAGCUUUA | |
| rno- | MIMAT00 | CCCUGAACUAGGGGUCUGGAGA | SEQ ID |
| miR- | 04655 | | NO: 807 |
| 345-3p | | | |
| rno- | MIMAT00 | UGUCCCUCUGGGUCGCCCA | SEQ ID |
| miR- | 00598 | | NO: 808 |
| 347 | | | |
| rno- | MI00006 | GAGCCCCUGCUGGUGGGCGCGGGGCGGGGGUUUCAGGUGGU | SEQ ID |
| miR- | 35 | CUCGCGGUGGCCGCCCGACUGUCCCUCUGGGUCGCCCAGCUG | NO: 809 |
| 347 | | GGGAGUUCC | |
| rno- | MIMAT00 | UUCACAAAGCCCAUACACUUUCAC | SEQ ID |
| miR- | 00604 | | NO: 810 |
| 350 | | | |
| rno- | MI00006 | CAUGGCACCUCCAUUUCCCUGAGGAGCCCUUUGAGCCUGAGG | SEQ ID |
| miR- | 42 | UGAAAAAAAAACAGGUCAAGAGGCGCCUGGGAACUGGAG | NO: 811 |
| 351 | | | |
| rno- | MIMAT00 | AGAGUAGUAGGUUGCAUAGUA | SEQ ID |
| miR- | 00610 | | NO: 812 |
| 352 | | | |
| rno- | MI00035 | AGCGAGGUUGCCCUUUGUAUAUUCGGUUUAUUGACAUGGG | SEQ ID |
| miR- | 46 | AUAUACAAGGGCAAGCUCUCU | NO: 813 |
| 381 | | | |
| rno- | MIMAT00 | AAUGUUGCUCGGUGAACCCC | SEQ ID |
| miR- | 03205 | | NO: 814 |
| 409a- | | | |
| 3p | | | |
| rno- | MI00061 | CAUGUGUAUAUAUGUGUGUGUGUAUGUCCAUGUGUGUAUA | SEQ ID |
| miR- | 12 | UGAAUAUACAUACACACACACAUACACACACGUGCAAGCACAC | NO: 815 |
| 466b | | ACA | |
| rno- | MI00034 | ACUGCUACAGUGGCAGCUUGGUUGUCGUAUGCGUGAUGACA | SEQ ID |
| miR- | 77 | CGUUCUCGUGUAUUCCAGAAUGACAUCACAUAUAUGGCAGC | NO: 816 |
| 489 | | UAAACUGUUACAGGAACAACAAGU | |
| rno- | MIMAT00 | CAUGCCUUGAGUGUAGGACUGU | SEQ ID |
| miR- | 05322 | | NO: 817 |
| 532-5p | | | |
| rno- | MIMAT00 | GUGUCUGUUUCCUGCCGGA | SEQ ID |
| miR- | 12837 | | NO: 818 |
| 632 | | | |
| rno- | MI00037 | CUGGCUGGGGAAAAAGAUUGGAUAGAAAACAUUAUUCUAUU | SEQ ID |
| miR- | 22 | CAUUUACUCCCCAGCCUA | NO: 819 |
| 664 | | | |
| rno- | MI00006 | UGUUGGCCUAGUUCUGUGUGGAAGACUAGUGAUUUUGUUG | SEQ ID |
| miR-7 | 41 | UUUUUAGAUAACUAAGACGACAACAAAUCACAGUCUGCCAUA | NO: 820 |
| | | UGGCACAGGCCACCU | |
| rno- | MI00061 | UGCAGUGCUGUAUUCAGAUUGGUGCCUGUCAUGUUUAUAA | SEQ ID |
| miR- | 62 | GAAUGAAAGACGCCAAACUGGGUAGAGUGGAGCUCA | NO: 821 |
| 743a | | | |
| rno- | MI00061 | GGUGCGUGAGGUGGUUGACCAGAGAGCACACGCUAUAUUUG | SEQ ID |
| miR- | 63 | UGCCGUUUGUGACCUGGUCCACUAACCCUCAGUAUCU | NO: 822 |
| 758 | | | |
| rno- | MIMAT00 | GCAUGACACCAUACUGGGUAGA | SEQ ID |
| miR- | 05286 | | NO: 823 |
| 878 | | | |
| rno- | MI00061 | UGCAGUACAAUAUUCAGAGUGGUAGCAGUCACUUUAUUCUA | SEQ ID |
| miR- | 23 | AAGUAACUGUGGCAUUUCUGAAUAGAGUAAUGUUCA | NO: 824 |
| 881 | | | |
| rno- | MI00008 | CCCAUUGGCAUAAACCCGUAGAUCCGAUCUUGUGGUGAAGU | SEQ ID |
| miR- | 83 | GGACCGCACAAGCUCGUUUCUAUGGGUCUGUGGCAGUGUG | NO: 825 |
| 99a | | | |
| rnu44 | NR_0027 | CCUGGAUGAUGAUAGCAAAUGCUGACUGAACAUGAAGGUCU | SEQ ID |
| | 50 | UAAUUAGCUCUAACUGACU | NO: 826 |
| rnu48 | NR_0027 | GAUGACCCCAGGUAACUCUGAGUGUGUCGCUGAUGCCAUCAC | SEQ ID |
| | 45 | CGCAGCGCUCUGACC | NO: 827 |
| snorna | AF357323 | CUAAAAUAGCUGGAAUUACCGGCAGAUUGGUAGUGGUGAGC | SEQ ID |
| 135 | | CUAUGGUUUUCUGAAG | NO: 828 |
| snorna | AF357327 | GCUGUACUGACUUGAUGAAAGUACUUUUGAACCCUUUUCCA | SEQ ID |
| 202 | | UCUGAUG | NO: 829 |
| u6snrn | NR_0043 | GUGCUCGCUUCGGCAGCACAUAUACUAAAAUUGGAACGAUAC | SEQ ID |
| a | 94 | AGAGAAGAUUAGCAUGGCCCCUGCGCAAGGAUGACACGCAAA | NO: 830 |
| | | UUCGUGAAGCGUUCCAUAUUUUUACUGCCCUCCAUGCCCUGC | |
| | | CCCACAAACGCUCUGAUAACAGUCUGUCCCUGUCUCUCUCCU | |
| | | GCUGCUCCUAUGGAAGCGAAGUUUUCCGCUCCUGCAGAAAGC | |
| | | AAAGUUACGACUCAGAGACGGCUGAGGAUGACAUCAGCGAU | |
| | | GUGC | |
| u87 | NR_0025 | ACAAUGAUGACUUAAAUUACUUUUUGCCGUUUACCCAGCUG | SEQ ID |
| | 98 | AGGUUGUCUUUGAAGAAAUAAUUUUAAGACUGAGA | NO: 831 |
| y1 | MGI9799 | AAAGACUAGUCAAGUGCAGUAGUGAGAAGGGGGGAAAGUGU | SEQ ID |
| | 5 | AGAACAGGAGUUCAAUCUGUAACUGACUGUGAACAAUCAAU | NO: 832 |
| | | UGAGAUAACUCACUACCUUCGGACCAGCC | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of diagnosing neuropathic pain in a subject, the method comprising:
a. determining the level of at least one microRNA in a biological sample of the subject, wherein the at least one microRNA comprises at least one microRNA selected from the group consisting of hsa-miR-31, hsa-miR-636, and hsa-miR-16-1# or at least one exosomal miRNA selected from the group consisting of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201,
b. comparing the level of the at least one microRNA in the biological sample with the level of the at least one miRNA or in a comparator, wherein when the level of the at least one microRNA in the biological sample is different than the level of the at least one miRNA in the comparator, the subject is diagnosed with neuropathic pain and the subject is administered a treatment for neuropathic pain.

2. The method of claim 1, wherein the neuropathic pain is complex regional pain syndrome (CRPS).

3. The method of claim 1, wherein the treatment for neuropathic pain is a NMDA receptor antagonist selected from the group consisting of ketamine, memantine, dizocilpine, phencyclidine, APV (AP5), amantadine, dextromethorphan, dextrorphan, AP7, riluzole, tiletamine, midafotel, aptiganel, methoxetamine, MK-801, ifenprodil, conantokin, and NVP-AAM077.

4-35. (canceled)

36. A method of predicting the responsiveness of a treatment of neuropathic pain in a subject, the method comprising:
a. determining the level of at least one microRNA in a biological sample of the subject,
b. comparing the level of the at least one microRNA in the biological sample with the level of the at least one miRNA or in a comparator, wherein when the level of the at least one microRNA in the biological sample is different than the level of the at least one miRNA in the comparator, the subject is predicted to respond to treatment of neuropathic pain and treatment of the subject for neuropathic pain is initiated.

37. The method of claim 36, wherein the neuropathic pain is complex regional pain syndrome (CRPS).

38. The method of claim 36, wherein the at least one microRNA is at least one selected from the group consisting of hsa-miR-197, hsa-miR-150, hsa-miR-186, hsa-miR-10b, hsa-miR-605, hsa-miR-597, hsa-miR-410, hsa-miR-337-5p, hsa-miR-548d-5p, hsa-miR-548E, hsa-miR-21#, hsa-miR-7-2#, hsa-miR-182, hsa-miR-34a, hsa-miR-376a, hsa-miR-149, hsa-miR-504, hsa-miR-941, hsa-miR-493, hsa-miR-146a, hsa-miR-127-3p, hsa-miR-130a, and hsa-miR-450a.

39. The method of claim 38, wherein the treatment is administration of an NMDA receptor antagonist.

40. The method of claim 39, wherein the NMDA receptor antagonist is selected from the group consisting of ketamine, memantine, dizocilpine, phencyclidine, APV (AP5), amantadine, dextromethorphan, dextrorphan, AP7, riluzole, tiletamine, midafotel, aptiganel, methoxetamine, MK-801, ifenprodil, conantokin, and NVP-AAM077.

41. The method of claim 36, wherein the subject is human.

42. (canceled)

43. The method of claim 36, wherein the comparator is at least one comparator selected from the group consisting of a positive control, a negative control, a normal control, a wild-type control, a historical control, and a historical norm.

44. The method of claim 36, wherein the comparator is a control known to not to respond to the treatment.

45. The method of claim 36, wherein the level of the at least one miRNA is higher than the level of the at least one miRNA in the comparator by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%.

46. The method of claim 36, wherein the level of the at least one miRNA is lower than the level of the at least one miRNA in the comparator by at least 10%, by at least 20%, by at least 30%, by at least 40%, by at least 50%, by at least 60%, by at least 70%, by at least 80%, by at least 90%, or by at least 100%.

47. The method of claim 36, wherein determining the level of the at least one microRNA utilizes at least one technique selected from the group consisting of reverse transcription, PCR and a microarray.

48. A method of treating neuropathic pain comprising administering to a subject in need thereof an effective amount of a therapeutic agent that modulates the expression and/or activity of at least one miRNA selected from the group consisting of hsa-miR-337-3p, hsa-miR-605, hsa-miR-597, RNU44.A, and hsa-miR-650 or at least one exosomal miRNA selected from the group consisting of miR-21#, miR-146b, miR-126-5p, miR-146a, miR-200c, miR-204, miR-212, miR-674, miR-222, miR-342-3p, miR-24, miR-27a, miR-878-3p, let-7b, miR-347, miR-155, miR-532-3p, miR-320, miR-146b, miR-24-2#, miR-29c, miR-7#, miR-326, miR-720, miR-93#, miR-27a#, miR-671-3p, miR-327, miR-489, miR-23a#, miR-99a#, miR-199a-3p, miR-939, miR-25#, let-7a, let-7b, let-7c, miR-320B, miR-126, miR-629.A, miR-664, miR-320, miR-1285, miR-625#, miR-532-3p, miR-181a-2#, RNU48, miR-720, RNU44, and miR-1201.

49. The method of claim 48, the neuropathic pain is complex regional pain syndrome (CRPS).

50. The method of claim 48, wherein the subject is human.

51. The method of claim 48, wherein the therapeutic agent inhibits the expression and/or activity of the at least one miRNA.

52. The method of claim 48, wherein the therapeutic agent enhances the expression and/or activity of the at least one miRNA.

53. The method of claim 48, wherein the therapeutic agent comprises at least one selected from the group consisting of a small molecule, antibody, antibody fragment, peptide, peptidomimetic, nucleic acid, antisense molecule, miRNA, and ribozyme.

54-85. (canceled)
